(19) United States
(12) Patent Application Publication
(10) Pub. No.: US 2024/0032418 A1
(43) Pub. Date: Jan. 25, 2024

(54) PLURALITY OF HOST MATERIALS, ORGANIC ELECTROLUMINESCENT COMPOUND, AND ORGANIC ELECTROLUMINESCENT DEVICE COMPRISING THE SAME

(71) Applicant: ROHM AND HAAS ELECTRONIC MATERIALS KOREA LTD., Chungcheongnam-do (KR)

(72) Inventors: Yea-Mi SONG, Gyeonggi-do (KR); Doo-Hyeon MOON, Gyeonggi-do (KR); Hyo-Jung LEE, Gyeonggi-do (KR); Hyun-Ju KANG, Gyeonggi-do (KR); Kyoung-Jin PARK, Gyeonggi-do (KR); Mi-Ja LEE, Gyeonggi-do (KR); DaiKyu KIM, Gyeonggi-do (KR)

(21) Appl. No.: 18/329,270

(22) Filed: Jun. 5, 2023

(30) Foreign Application Priority Data
Jul. 11, 2022 (KR) 10-2022-0085014
May 12, 2023 (KR) 10-2023-0061568

(51) Int. Cl. H10K 85/60; C07D 413/14; C09K 11/06

(52) U.S. Cl. CPC H10K 85/657; C07D 413/14; C09K 11/06; H10K 85/6574; H10K 85/654; H10K 85/6572; H10K 50/12

(57) ABSTRACT

The present disclosure relates to a plurality of host materials, an organic electroluminescent compound, and an organic electroluminescent device comprising the same. An organic electroluminescent device having improved driving voltage, luminous efficiency, and/or lifespan properties can be provided by including the organic electroluminescent compound according to the present disclosure as an organic electroluminescent material or by including a specific combination of compounds according to the present disclosure as a plurality of host materials.

### TECHNICAL FIELD

The present disclosure relates to an organic plurality of host materials, an organic electroluminescent compound, and an organic electroluminescent device comprising the same.

### BACKGROUND ART

In 1987, Tang et al. of Eastman Kodak first developed a small molecular green organic electroluminescent device (OLED) by using TPD/Alq₃ bilayer consisting of a light-emitting layer and a charge transport layer. Thereafter, the development of OLEDs was rapidly effected and OLEDs have been commercialized. Currently, OLEDs mainly use phosphorescent materials having excellent luminous efficiency in panel implementation. Thus, OLEDs having high luminous efficiency and/or long lifespan are required for long-term use and high resolution of a display.

In order to improve luminous efficiency, driving voltage, and/or lifespan, various materials or concepts for an organic layer of an organic electroluminescent device have been proposed. However, they were not satisfied in practical use. Accordingly, there has been a continuous need to develop organic electroluminescent devices having more improved performances, for example, improved driving voltage, luminous efficiency, power efficiency, and/or lifespan properties, compared to organic electroluminescent devices previously disclosed.

Meanwhile, Korean Patent Application Laid-Open No. 2020-0035905 discloses a compound having a structure in which benzoxazole or benzothiazole is bonded to pyrimidine or triazine via a linker as a basic backbone. However, the aforementioned reference fails to specifically disclose an organic electroluminescent compound, and a plurality of host materials comprising a specific combination of compounds claimed herein. In addition, there has been a need to develop a material having improved performances, for example, low driving voltage, high luminous efficiency, and/or long lifespan properties, compared to the compound disclosed in the aforementioned references.

### DISCLOSURE OF INVENTION

### Technical Problems

The objective of the present disclosure is to provide an improved plurality of host materials capable of providing an organic electroluminescent device with improved driving voltage, luminous efficiency and/or lifespan properties. Another objective of the present disclosure is to provide an organic electroluminescent compound having a novel structure suitable for application to an organic electroluminescent device. Still another objective of the present disclosure is to provide an organic electroluminescent device with improved driving voltage, luminous efficiency, and/or lifespan properties by including a compound or a specific combination of compounds of the present disclosure.

### Solution to Problem

As a result of intensive research to solve the above technical problems, the present inventors found that the above objectives can be achieved by a plurality of host materials comprising at least one first host compound and at least one second host compound, wherein the first host compound is represented by the following formula 1, and wherein the second host compound is represented by the following formula 2. In addition, the present inventors found that the above objectives can be achieved by a compound is represented by the following formula 3.

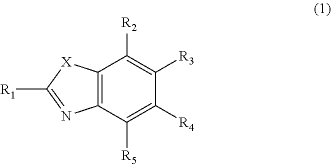

In formula 1,
X represents O or S;
R₁ to R₅ each independently represent hydrogen, deuterium, a substituted or unsubstituted (C6-C30)aryl, or the following formula a; with a proviso that at least one of R₁ to R₅ represents formula a;

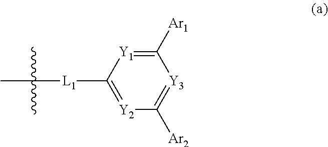

in formula a,
L₁ represents a single bond, a substituted or unsubstituted (C6-C30)arylene, or a substituted or unsubstituted (3- to 30-membered)heteroarylene;
Y₁ to Y₃ each independently represent N or CH; with a proviso that at least two of Y₁ to Y₃ represent N; and
Ar₁ and Ar₂ each independently represent a substituted or unsubstituted (C1-C30)alkyl, a substituted or unsubstituted (C6-C30)aryl, or a substituted or unsubstituted (3- to 30-membered)heteroaryl;

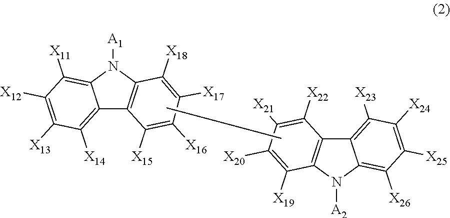

in formula 2,
A₁ and A₂ each independently represent a substituted or unsubstituted (C6-C30)aryl, a substituted or unsubstituted dibenzofuranyl, a substituted or unsubstituted dibenzothiophenyl, or a substituted or unsubstituted carbazolyl;
any one of X₁₅ to X₁₈ and any one of X₁₉ to X₂₂ are linked to each other to form a single bond; and
X₁₁ to X₁₄, X₂₃ to X₂₆, and X₁₅ to X₂₂ not forming the single bond each independently represent hydrogen, deuterium, a substituted or unsubstituted (C6-C30)aryl, or a substituted or unsubstituted (3- to 30-membered)heteroaryl; or may be linked to an adjacent substituent(s) to form a ring(s).

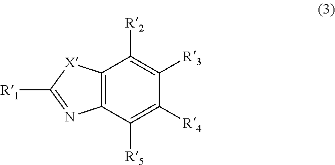

In formula 3,
X represent O or S;
R′₁ represents hydrogen, deuterium, or a phenyl unsubstituted or substituted with deuterium;
R′₂ to R′₅ each independently represent hydrogen, deuterium, a substituted or unsubstituted (C6-C30)aryl, or the following formula a′; with a proviso that at least one of R′₂ to R′₅ represents formula a′;

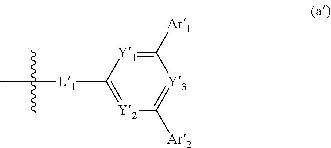

in formula a′,
L′₁ represents a single bond, or a substituted or unsubstituted (C6-C30)arylene;
Y′₁ to Y′₃ each independently represent N or CH; with a proviso that at least two of Y₁ to Y′₃ represent N;
Ar′₁ and Ar′₂ each independently represent a substituted or unsubstituted (C6-C30)aryl, a substituted or unsubstituted carbazolyl, a substituted or unsubstituted dibenzofuranyl, or a substituted or unsubstituted dibenzothiophenyl; with a proviso that at least one of Ar′₁ and Ar′₂ is a substituted or unsubstituted carbazolyl, a substituted or unsubstituted dibenzofuranyl, or a substituted or unsubstituted dibenzothiophenyl; and
Ar′₁ and Ar′₂ are different from each other.

### Advantageous Effects of Invention

The plurality of host materials and the organic electroluminescent compound according to the present disclosure exhibit suitable performance for using in organic electroluminescent devices. In addition, an organic electroluminescent device having lower driving voltage, higher luminous efficiency, and/or longer lifespan properties compared to a conventional organic electroluminescent device is provided by including the organic electroluminescent compound according to the present disclosure as an organic electroluminescent material or by including a specific combination of compounds according to the present disclosure as a plurality of host materials, and it is possible to manufacture a display device or a lighting device using the same.

### MODE FOR THE INVENTION

Hereinafter, the present disclosure will be described in detail. However, the following description is intended to explain the present disclosure, and is not meant to restrict the scope of the present disclosure.

The term “organic electroluminescent compound” in the present disclosure means a compound that may be used in an organic electroluminescent device, and may be comprised in any layer constituting an organic electroluminescent device, as necessary.

The term “organic electroluminescent material” in the present disclosure means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron blocking material, a light-emitting material (including a host material and a dopant material), an electron buffer material, a hole blocking material, an electron transport material, an electron injection material, etc.

The term “a plurality of host materials” in the present disclosure means a host material(s) comprising a combination of at least two compounds, which may be comprised in any light-emitting layer constituting an organic electroluminescent device. It may mean both a material before being comprised in an organic electroluminescent device (for example, before vapor deposition) and a material after being comprised in an organic electroluminescent device (for example, after vapor deposition). For example, a plurality of host materials of the present disclosure may be a combination of two or more host materials that may optionally further comprise a conventional material included in an organic electroluminescent material. Two or more compounds included in the plurality of host materials of the present disclosure may be together included in one light-emitting layer or may be respectively included in different light-emitting layers. For example, the two or more host materials may be mixture-evaporated or co-evaporated, or may be individually evaporated.

Herein, the term “(C1-C30)alkyl” is meant to be a linear or branched alkyl having 1 to 30 carbon atoms constituting the chain, in which the number of carbon atoms is preferably 1 to 10, and more preferably 1 to 6. The above alkyl may include methyl, ethyl, n-propyl, isopropyl, n-butyl, isobutyl, tert-butyl, sec-butyl, etc. The term “(C3-C30)cycloalkyl” or “(C3-C30)cycloalkylene” is meant to be a mono- or polycyclic hydrocarbon having 3 to 30 ring backbone carbon atoms, in which the number of carbon atoms is preferably 3 to 20, and more preferably 3 to 7. The above cycloalkyl may include cyclopropyl, cyclobutyl, cyclopentyl, cyclohexyl, cyclopentylmethyl, cyclohexylmethyl, etc. The term “(3- to 7-membered)heterocycloalkyl” is meant to be a cycloalkyl having 3 to 7 ring backbone atoms, and including at least one heteroatom selected from the group consisting of B, N, O, S, Si, and P, preferably the group consisting of O, S, and N. The above heterocycloalkyl may include tetrahydrofuran, pyrrolidine, thiolane, tetrahydropyran, etc. The term “(C6-C30)aryl” or “(C6-C30)arylene” is meant to be a monocyclic or fused ring radical derived from an aromatic hydrocarbon having 6 to 30 ring backbone carbon atoms. The above aryl and arylene may be partially saturated; and may comprise a spiro structure. The above aryl may include phenyl, biphenyl, terphenyl, naphthyl, binaphthyl, phenylnaphthyl, naphthylphenyl, fluorenyl, phenylfluorenyl, diphenylfluorenyl, benzofluorenyl, dibenzofluorenyl, phenanthrenyl, phenylphenanthrenyl, benzophenanthrenyl, anthracenyl, indenyl, triphenylenyl, pyrenyl, tetracenyl, perylenyl, chrysenyl, naphthacenyl, fluoranthenyl, spirobifluorenyl, spiro[fluorene-benzofluoren]yl, spiro[cyclopenten-fluoren]yl, spiro[dihydroinden-fluoren]yl, azulenyl, tetramethyldihydrophenanthrenyl, etc. Specifically, the aryl may include phenyl, 1-naphthyl, 2-naphthyl, 1-anthryl, 2-anthryl, 9-anthryl, benzanthryl, 1-phenanthryl, 2-phenanthryl, 3-phenanthryl, 4-phenanthryl, 9-phenanthryl, naphthacenyl, pyrenyl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 1-fluorenyl, 2-fluorenyl, 3-fluorenyl, 4-fluorenyl, 9-fluorenyl, benzo[a]fluorenyl, benzo[b]fluorenyl, benzo[c]fluorenyl, dibenzofluorenyl, 2-biphenylyl, 3-biphenylyl, 4-biphenylyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 3-fluoranthenyl, 4-fluoranthenyl, 8-fluoranthenyl, 9-fluoranthenyl, benzofluoranthenyl, o-tolyl, m-tolyl, p-tolyl, 2,3-xylyl, 3,4-xylyl, 2,5-xylyl, mesityl, o-cumenyl, m-cumenyl, p-cumenyl, p-tert-butylphenyl, p-(2-phenylpropyl)phenyl, 4′-methylbiphenyl, 4″-tert-butyl-p-terphenyl-4-yl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, 9,9,10,10-tetramethyl-9,10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc.

The term “(3- to 30-membered)heteroaryl” or “(3- to 30-membered)heteroarylene” means an aryl group or an arylene group having 3 to 30 ring backbone atoms and including at least one, preferably 1 to 4 heteroatom(s) selected from the group consisting of B, N, O, S, Si, and P. The above heteroaryl(ene) may be a monocyclic ring or a fused ring condensed with at least one benzene ring; may be partially saturated; may be one formed by linking at least one heteroaryl or aryl group to a heteroaryl group via a single bond(s); and may comprise a spiro structure. The above heteroaryl may include a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl such as benzofuranyl, benzothiophenyl, isobenzofuranyl, dibenzofuranyl, dibenzothiophenyl, dibenzoselenophenyl, naphthobenzofuranyl, naphthobenzothiophenyl, benzofuroquinolinyl, benzofuroquinazolinyl, benzofuronaphthyridinyl, benzofuropyrimidinyl, naphthofuropyrimidinyl, benzothienoquinolinyl, benzothienoquinazolinyl, benzothienonaphthyridinyl, benzothienopyrimidinyl, naphthothienopyrimidinyl, pyrimidoindolyl, benzopyrimidoindolyl, benzofuropyrazinyl, naphthofuropyrazinyl, benzothienopyrazinyl, naphthothienopyrazinyl, pyrazinoindolyl, benzopyrazinoindolyl, benzimidazolyl, benzothiazolyl, benzoisothiazolyl, benzoisoxazolyl, benzoxazolyl, isoindolyl, indolyl, indazolyl, benzothiadiazolyl, quinolyl, isoquinolyl, cinnolinyl, quinazolinyl, quinoxalinyl, carbazolyl, benzocarbazolyl, dibenzocarbazolyl, phenoxazinyl, phenanthridinyl, benzodioxolyl, dihydroacridinyl, benzotriazolyl, phenazinyl, imidazopyridyl, chromenoquinazolinyl, thiochromenoquinazolinyl, dimethylbenzopyrimidinyl, indolocarbazolyl, indenocarbazolyl, etc. More specifically, the heteroaryl may include 1-pyrrolyl, 2-pyrrolyl, 3-pyrrolyl, pyrazinyl, 2-pyridyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolidinyl, 2-indolidinyl, 3-indolidinyl, 5-indolidinyl, 6-indolidinyl, 7-indolidinyl, 8-indolidinyl, 2-imidazopyridyl, 3-imidazopyridyl, 5-imidazopyridyl, 6-imidazopyridyl, 7-imidazopyridyl, 8-imidazopyridyl, 3-pyridyl, 4-pyridyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazolyl-1-yl, azacarbazolyl-2-yl, azacarbazolyl-3-yl, azacarbazolyl-4-yl, azacarbazolyl-5-yl, azacarbazolyl-6-yl, azacarbazolyl-7-yl, azacarbazolyl-8-yl, azacarbazolyl-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-tert-butylpyrrol-4-yl, 3-(2-phenylpropyl)pyrrol-1-yl, 2-methyl-1-indolyl, 4-methyl-1-indolyl, 2-methyl-3-indolyl, 4-methyl-3-indolyl, 2-tert-butyl-1-indolyl, 4-tert-butyl-1-indolyl, 2-tert-butyl-3-indolyl, 4-tert-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtho-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2,3-b]-benzofuranyl, 10-naphtho-[2,3-b]-benzofuranyl, 1-naphtho-[2,1-b]-benzofuranyl, 2-naphtho-[2,1-b]-benzofuranyl, 3-naphtho-[2,1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2,1-b]-benzothiophenyl, 3-naphtho-[2,1-b]-benzothiophenyl, 4-naphtho-[2,1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 2-benzofuro[3,2-d]pyrimidinyl, 6-benzofuro[3,2-d]pyrimidinyl, 7-benzofuro[3,2-d]pyrimidinyl, 8-benzofuro[3,2-d]pyrimidinyl, 9-benzofuro[3,2-d]pyrimidinyl, 2-benzothio[3,2-d]pyrimidinyl, 6-benzothio[3,2-d]pyrimidinyl, 7-benzothio[3,2-d]pyrimidinyl, 8-benzothio[3,2-d]pyrimidinyl, 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-benzothio[3,2-d]pyrazinyl, 6-benzothio[3,2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc. In the present disclosure, “halogen” includes F, Cl, Br, and I.

In addition, “ortho (o-),” “meta (m-),” and “para (p-)” are prefixes, which represent the relative positions of substituents, respectively. Ortho indicates that two substituents are adjacent to each other, and for example, when two substituents in a benzene derivative occupy positions 1 and 2, it is called an ortho position. Meta indicates that two substituents are at positions 1 and 3, and for example, when two substituents in a benzene derivative occupy positions 1 and 3, it is called a meta position. Para indicates that two substituents are at positions 1 and 4, and for example, when two substituents in a benzene derivative occupy positions 1 and 4, it is called a para position.

The term “a ring formed by a linkage of adjacent substituents” means that at least two adjacent substituents are linked to or fused with each other to form a substituted or unsubstituted mono- or polycyclic (3- to 30-membered) alicyclic or aromatic ring, or the combination thereof. The ring may be preferably a substituted or unsubstituted mono- or polycyclic (3- to 26-membered) alicyclic or aromatic ring, or the combination thereof, more preferably a mono- or polycyclic (5- to 25-membered) aromatic ring unsubstituted or substituted with at least one of a (C6-C18)aryl(s) and a (3- to 20-membered)heteroaryl(s). In addition, the formed ring may contain at least one heteroatom selected from B, N, O, S, Si, and P, preferably at least one heteroatom selected from N, O, and S. For example, the ring may be a benzene ring, a cyclopentane ring, an indan ring, a fluorene ring, a phenanthrene ring, an indole ring, a carbazole ring, or a xanthene ring, etc.

“Substituted” in the expression “substituted or unsubstituted” means that a hydrogen atom in a certain functional group is replaced with another atom or another functional group (i.e., a substituent), and also includes that the hydrogen atom is replaced with a group formed by a linkage of two or more substituents of the above substituents. For example, the “group formed by a linkage of two or more substituents” may be pyridine-triazine. That is, pyridine-triazine may be interpreted as one heteroaryl substituent or as substituents in which two heteroaryl substituents are linked. In the present disclosure, the substituent(s) of the substituted alkyl, the substituted aryl(ene), the substituted heteroaryl(ene), the substituted dibenzofuranyl, the substituted dibenzothiophenyl, and the substituted carbazolyl, each independently, are at least one selected from the group consisting of deuterium; a halogen; a cyano; a carboxyl; a nitro; a hydroxyl; a phosphine oxide; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (3- to 30-membered)heteroaryl unsubstituted or substituted with at least one of deuterium and a (C6-C30)aryl(s); a (C6-C30)aryl unsubstituted or substituted with deuterium; a tri(C1-C30)alkylsilyl; a tri(C6-C30)arylsilyl; a di(C1-C30)alkyl(C6-C30)arylsilyl; a (C1-C30)alkyldi(C6-C30)arylsilyl; an amino; a mono- or di-(C1-C30)alkylamino; a mono- or di-(C2-C30)alkenylamino; a mono- or di-(C6-C30)arylamino; a mono- or di-(3- to 30-membered)heteroarylamino; a (C1-C30)alkyl(C2-C30)alkenylamino; a (C1-C30)alkyl(C6-C30)arylamino; a (C1-C30)alkyl(3- to 30-membered)heteroarylamino; a (C2-C30)alkenyl(C6-C30)arylamino; a (C2-C30)alkenyl(3- to 30-membered)heteroarylamino; a (C6-C30)aryl(3- to 30-membered)heteroarylamino; a (C1-C30)alkylcarbonyl; a (C1-C30)alkoxycarbonyl; a (C6-C30)arylcarbonyl; a (C6-C30)arylphosphine; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl. According to one embodiment of the present disclosure, the substituent(s), each independently, are at least one selected from the group consisting of deuterium; a (6- to 25-membered)heteroaryl unsubstituted or substituted with at least one of deuterium and a (C6-C18)aryl(s); and a (C6-C28)aryl unsubstituted or substituted with deuterium. According to another embodiment of the present disclosure, the substituent(s), each independently, are at least one selected from the group consisting of deuterium; a (6- to 20-membered)heteroaryl unsubstituted or substituted with at least one of deuterium and a (C6-C12)aryl(s); and a (C6-C20)aryl unsubstituted or substituted with deuterium. For example, the substituent(s), each independently, may be at least one selected from the group consisting of deuterium, a phenyl, a biphenyl, a terphenyl, a naphthyl, a triphenylenyl, a dibenzofuranyl, a dibenzothiophenyl, and a carbazolyl unsubstituted or substituted with a phenyl(s), wherein the above substituents may be further substituted with deuterium.

A plurality of host materials according to the present disclosure comprise a first host material and a second host material, wherein the first host material comprises at least one compound represented by formula 1, and the second host material comprises at least one compound represented by formula 2.

Hereinafter, the compound represented by formula 1 will be described in more detail.

In formula 1, X represents O or S.

In formula 1, R₁ to R₅ each independently represent hydrogen, deuterium, a substituted or unsubstituted (C6-C30)aryl, or the formula a; with a proviso that at least one of R₁ to R₅ represents formula a. According to one embodiment of the present disclosure, R₁ to R₅ each independently represent hydrogen; deuterium; a (C6-C30)aryl unsubstituted or substituted with at least one of deuterium and a (C6-C30)aryl(s); or the formula a. According to another embodiment of the present disclosure, R₁ to R₅ each independently represent hydrogen; deuterium; a (C6-C18)aryl unsubstituted or substituted with deuterium; or formula a. According to still another embodiment of the present disclosure, any one of R₁ to R₅ represents formula a, and the others each independently represent hydrogen; deuterium; or a (C6-C12)aryl unsubstituted or substituted with deuterium. For example, R₁ to R₅, each independently, may represent hydrogen, deuterium, a phenyl unsubstituted or substituted with deuterium, or formula a, etc.; with a proviso that at least one of R₁ to R₅ is formula a.

L₁ represents a single bond, a substituted or unsubstituted (C6-C30)arylene, or a substituted or unsubstituted (3- to 30-membered)heteroarylene. According to one embodiment of the present disclosure, L₁ represents a single bond, or a (C6-C30)arylene unsubstituted or substituted with at least one of deuterium and a (C6-C30)aryl(s). According to another embodiment of the present disclosure, L₁ represents a single bond, or a (C6-C20)arylene unsubstituted or substituted with at least one of deuterium and a (C6-C20)aryl(s). For example, L₁ may represent a single bond, a phenylene unsubstituted or substituted with a phenyl(s), a biphenylene unsubstituted or substituted with a phenyl(s), or terphenylene, etc., which may be further substituted with deuterium.

Y₁ to Y₃ each independently represent N or CH; with a proviso that at least two of Y₁ to Y₃ represent N. For example, two of Y₁ to Y₃ represent N and the other represents CH; or Y₁ to Y₃ represent all N.

Ar₁ and Ar₂ each independently represent a substituted or unsubstituted (C1-C30)alkyl, a substituted or unsubstituted (C6-C30)aryl, or a substituted or unsubstituted (3- to 30-membered)heteroaryl. According to one embodiment of the present disclosure, Ar₁ and Ar₂ each independently represent a (C6-C30)aryl unsubstituted or substituted with at least one of deuterium, a (C6-C30)aryl(s), and a (3- to 30-membered)heteroaryl(s); or a (3- to 30-membered)heteroaryl unsubstituted or substituted with at least one of deuterium and a (C6-C30)aryl(s). According to another embodiment of the present disclosure, Ar₁ and Ar₂ each independently represent a (C6-C25)aryl unsubstituted or substituted with at least one of deuterium, a (C6-C20)aryl(s), and a (6- to 20-membered)heteroaryl(s); or a (6- to 15-membered)heteroaryl unsubstituted or substituted with at least one of deuterium and a (C6-C15)aryl(s). Specifically, Ar₁ and Ar₂, each independently, may be a phenyl unsubstituted or substituted with a dibenzofuranyl(s), a dibenzothiophenyl(s), a carbazolyl(s), or a 9-phenylcarbazolyl(s); a biphenyl; a terphenyl; a quaterphenyl; a naphthyl; a phenylnaphthyl; a naphthylphenyl; a pyridyl; a pyrimidinyl; a triazinyl; a dibenzofuranyl unsubstituted or substituted with a phenyl(s); a dibenzothiophenyl unsubstituted or substituted with a phenyl(s); or a carbazolyl unsubstituted or substituted with at least one of a phenyl(s) and a biphenyl(s), which may be further substituted with at least one deuterium. For example, Ar₁ and Ar₂, each independently, may be a phenyl unsubstituted or substituted with a dibenzofuranyl(s), a dibenzothiophenyl(s), a carbazolyl(s), or a 9-phenylcarbazolyl(s); a biphenyl; a terphenyl; a dibenzofuranyl unsubstituted or substituted with a phenyl(s); a dibenzothiophenyl unsubstituted or substituted with a phenyl(s); or a carbazolyl unsubstituted or substituted with at least one of a phenyl(s) and a biphenyl(s), which may be further substituted with at least one deuterium.

Hereinafter, the compound represented by formula 2 will be described in more detail.

In formula 2, A₁ and A₂ each independently represent a substituted or unsubstituted (C6-C30)aryl, a substituted or unsubstituted dibenzofuranyl, a substituted or unsubstituted dibenzothiophenyl, or a substituted or unsubstituted carbazolyl. According to one embodiment of the present disclosure, A₁ and A₂ each independently represent a substituted or unsubstituted (C6-C25)aryl, a substituted or unsubstituted dibenzofuranyl, a substituted or unsubstituted dibenzothiophenyl, or a substituted or unsubstituted carbazolyl, and the substituent(s) thereof may be each independently at least one of deuterium, a (C6-C30)aryl, and a (3- to 30-membered)heteroaryl. According to another embodiment of the present disclosure, A₁ and A₂ each independently represent a (C6-C25)aryl unsubstituted or substituted with at least one of deuterium, a (C6-C18)aryl(s), a dibenzofuranyl(s), a dibenzothiophenyl(s) and a carbazolyl(s); a dibenzofuranyl unsubstituted or substituted with at least one of deuterium and a (C6-C18)aryl(s); a dibenzothiophenyl unsubstituted or substituted with at least one of deuterium and a (C6-C18)aryl(s); or a carbazolyl unsubstituted or substituted with at least one of deuterium and a (C6-C18)aryl(s). Specifically, A₁ and A₂, each independently, may be a substituted or unsubstituted phenyl, a substituted or unsubstituted biphenyl, a substituted or unsubstituted terphenyl, a substituted or unsubstituted naphthyl, a substituted or unsubstituted fluorenyl, a substituted or unsubstituted benzofluorenyl, a substituted or unsubstituted triphenylenyl, a substituted or unsubstituted fluoranthenyl, a substituted or unsubstituted phenanthrenyl, a substituted or unsubstituted dibenzofuranyl, a substituted or unsubstituted carbazolyl, or a substituted or unsubstituted dibenzothiophenyl. For example, A₁ and A₂, each independently, may be a phenyl unsubstituted or substituted with a naphthyl(s), a triphenylenyl(s), a dibenzofuranyl(s) or a dibenzothiophenyl(s); a biphenyl; a naphthyl; a naphthylphenyl; a phenylnaphthyl; a terphenyl; a triphenylenyl; a dibenzofuranyl unsubstituted or substituted with a phenyl(s); a dibenzothiophenyl unsubstituted or substituted with a phenyl(s); or a carbazolyl substituted with a phenyl(s) or a naphthyl(s), which may be further substituted with deuterium.

In formula 2, any one of X₁₅ to X₁₈ and any one of X₁₉ to X₂₂ are linked to each other to form a single bond. X₁₁ to X₁₄, X₂₃ to X₂₆, and X₁₅ to X₂₂ not forming the single bond each independently represent hydrogen, deuterium, a substituted or unsubstituted (C6-C30)aryl, or a substituted or unsubstituted (3- to 30-membered)heteroaryl; or may be linked to an adjacent substituent(s) to form a ring(s). For example, X₁₁ to X₂₆, each independently, may be hydrogen or deuterium.

According to one embodiment of the present disclosure, at least one, preferably at least two, more preferably at least three, and still more preferably all of X₁₁, X₁₈, X₁₉ and X₂₆ may be deuterium.

According to one embodiment of the present disclosure, the deuterium substitution rate of X₁₁ to X₂₆ may be about 25% to about 100%, preferably about 35% to about 100%, more preferably about 45% to about 100%, and still more preferably about 55% to about 100%.

According to one embodiment of the present disclosure, formula 2 may be represented by at least one of the following formulas 2-1 to 2-8.

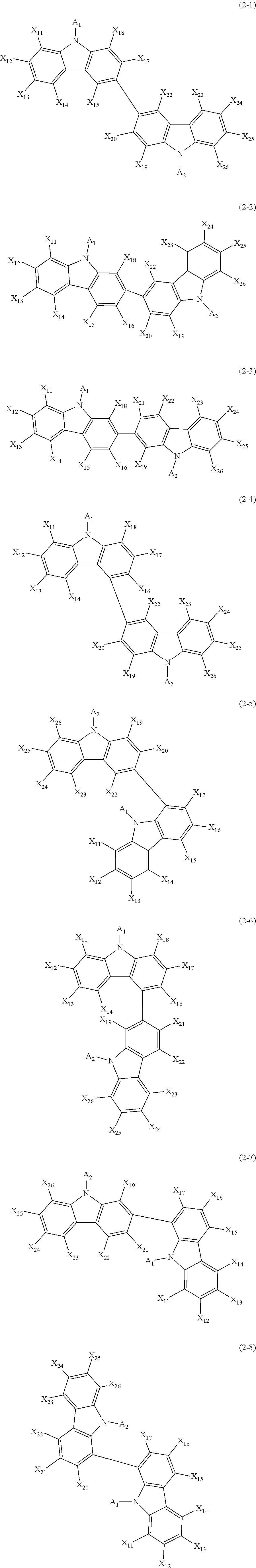

In formulas 2-1 to 2-8, A₁, A₂, and X₁₁ to X₂₆, and the preferred embodiments thereof are as defined in formula 2.

The compound represented by formula 1 may be at least one selected from the following compounds, but is not limited thereto.

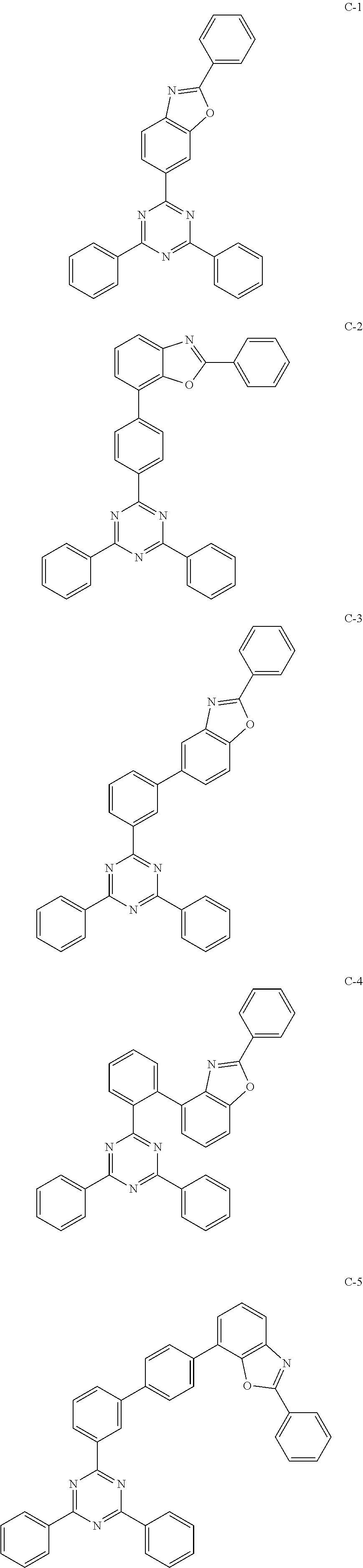 [SMILES: c1ccc(-c2nc(-c3ccccc3)nc(-c3ccc4nc(-c5ccccc5)oc4c3)n2)cc1 ; c1ccc(-c2nc(-c3ccccc3)nc(-c3ccc(-c4cccc5nc(-c6ccccc6)oc45)cc3)n2)cc1 ; c1ccc(-c2nc(-c3ccccc3)nc(-c3cccc(-c4ccc5oc(-c6ccccc6)nc5c4)c3)n2)cc1 ; c1ccc(-c2nc(-c3ccccc3)nc(-c3ccccc3-c3cccc4oc(-c5ccccc5)nc34)n2)cc1 ; c1ccc(-c2nc(-c3ccccc3)nc(-c3cccc(-c4ccc(-c5cccc6nc(-c7ccccc7)oc56)cc4)c3)n2)cc1]

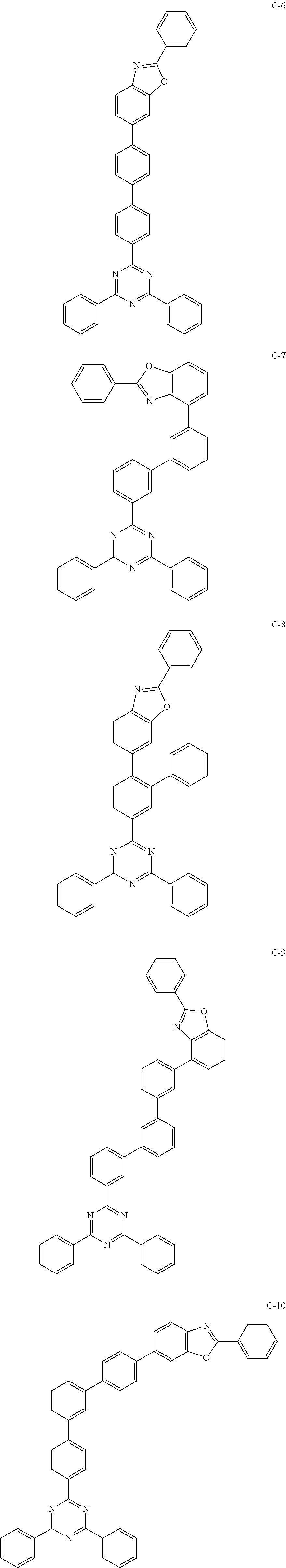 [SMILES: c1ccc(-c2nc(-c3ccccc3)nc(-c3ccc(-c4ccc(-c5ccc6nc(-c7ccccc7)oc6c5)cc4)cc3)n2)cc1 ; c1ccc(-c2nc(-c3ccccc3)nc(-c3cccc(-c4cccc(-c5cccc6oc(-c7ccccc7)nc56)c4)c3)n2)cc1 ; c1ccc(-c2nc(-c3ccccc3)nc(-c3ccc(-c4ccc5nc(-c6ccccc6)oc5c4)c(-c4ccccc4)c3)n2)cc1 ; c1ccc(-c2nc(-c3ccccc3)nc(-c3cccc(-c4cccc(-c5cccc(-c6cccc7oc(-c8ccccc8)nc67)c5)c4)c3)n2)cc1 ; c1ccc(-c2nc(-c3ccccc3)nc(-c3ccc(-c4cccc(-c5ccc(-c6ccc7nc(-c8ccccc8)oc7c6)cc5)c4)cc3)n2)cc1]

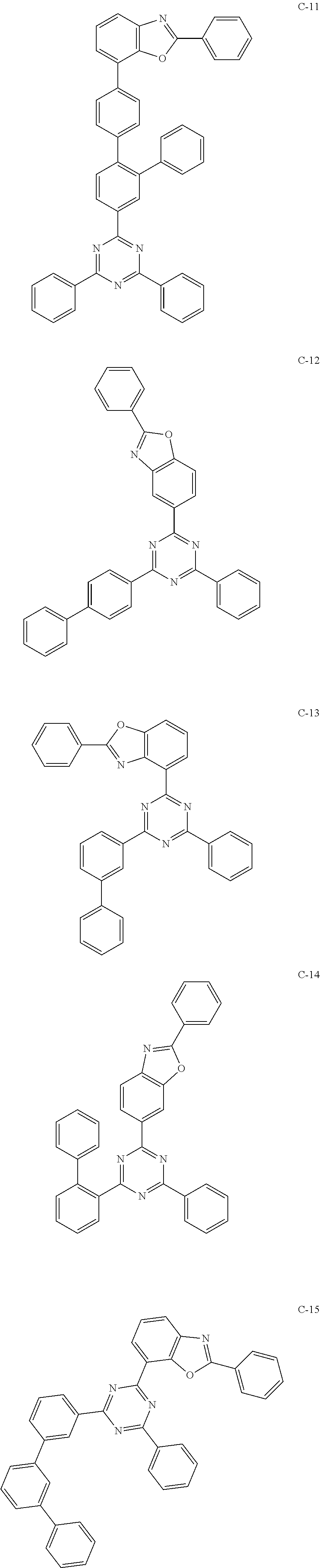 [SMILES: c1ccc(-c2nc(-c3ccccc3)nc(-c3ccc(-c4ccc(-c5cccc6nc(-c7ccccc7)oc56)cc4)c(-c4ccccc4)c3)n2)cc1 ; c1ccc(-c2ccc(-c3nc(-c4ccccc4)nc(-c4ccc5oc(-c6ccccc6)nc5c4)n3)cc2)cc1 ; c1ccc(-c2cccc(-c3nc(-c4ccccc4)nc(-c4cccc5oc(-c6ccccc6)nc45)n3)c2)cc1 ; c1ccc(-c2nc(-c3ccc4nc(-c5ccccc5)oc4c3)nc(-c3ccccc3-c3ccccc3)n2)cc1 ; c1ccc(-c2cccc(-c3cccc(-c4nc(-c5ccccc5)nc(-c5cccc6nc(-c7ccccc7)oc56)n4)c3)c2)cc1]

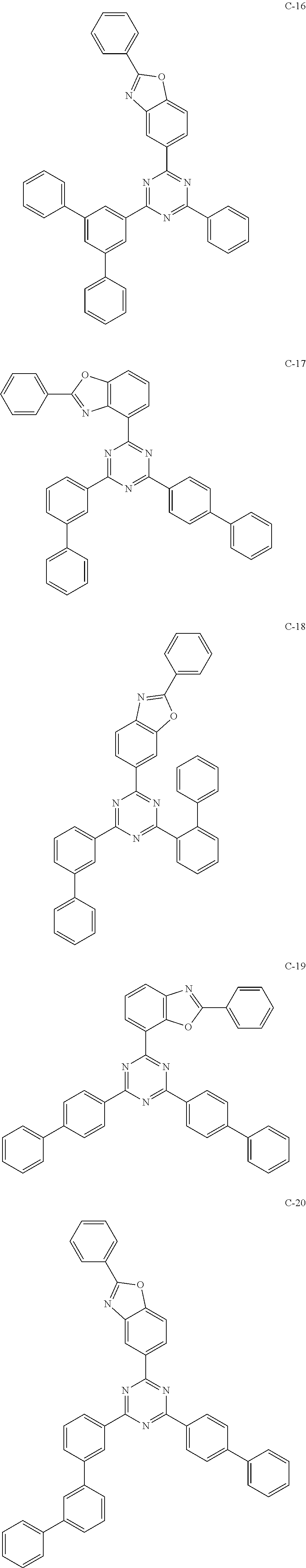 [SMILES: c1ccc(-c2cc(-c3ccccc3)cc(-c3nc(-c4ccccc4)nc(-c4ccc5oc(-c6ccccc6)nc5c4)n3)c2)cc1 ; c1ccc(-c2ccc(-c3nc(-c4cccc(-c5ccccc5)c4)nc(-c4cccc5oc(-c6ccccc6)nc45)n3)cc2)cc1 ; c1ccc(-c2cccc(-c3nc(-c4ccc5nc(-c6ccccc6)oc5c4)nc(-c4ccccc4-c4ccccc4)n3)c2)cc1 ; c1ccc(-c2ccc(-c3nc(-c4ccc(-c5ccccc5)cc4)nc(-c4cccc5nc(-c6ccccc6)oc45)n3)cc2)cc1 ; c1ccc(-c2ccc(-c3nc(-c4cccc(-c5cccc(-c6ccccc6)c5)c4)nc(-c4ccc5oc(-c6ccccc6)nc5c4)n3)cc2)cc1]

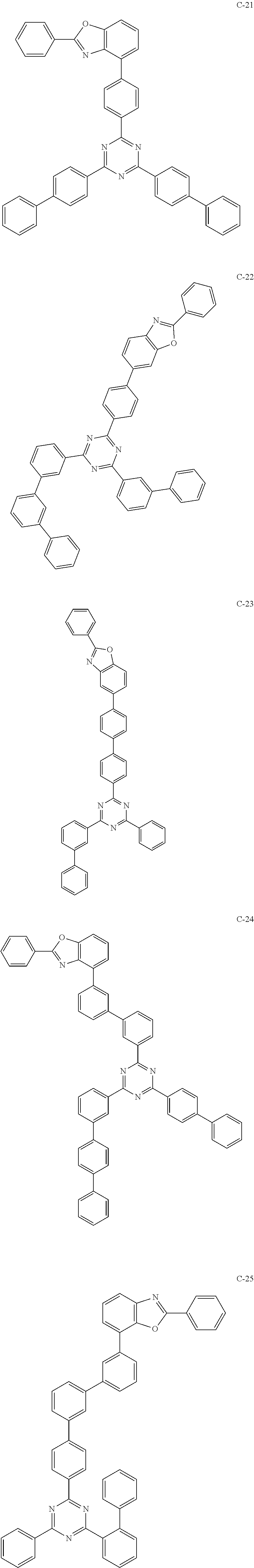

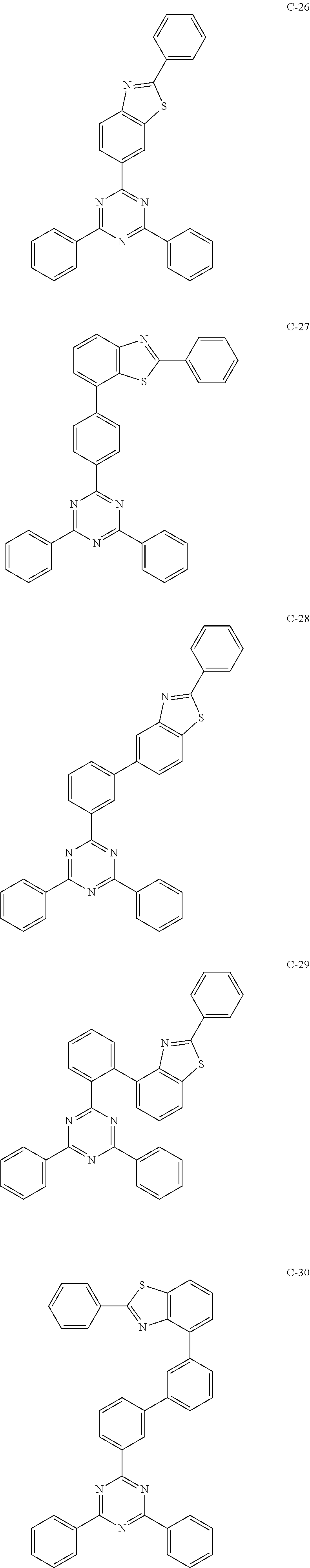 [SMILES: c1ccc(-c2nc(-c3ccccc3)nc(-c3ccc4nc(-c5ccccc5)sc4c3)n2)cc1 ; c1ccc(-c2nc(-c3ccccc3)nc(-c3ccc(-c4cccc5nc(-c6ccccc6)sc45)cc3)n2)cc1 ; c1ccc(-c2nc(-c3ccccc3)nc(-c3cccc(-c4ccc5sc(-c6ccccc6)nc5c4)c3)n2)cc1 ; c1ccc(-c2nc(-c3ccccc3)nc(-c3ccccc3-c3cccc4sc(-c5ccccc5)nc34)n2)cc1 ; c1ccc(-c2nc(-c3ccccc3)nc(-c3cccc(-c4cccc(-c5cccc6sc(-c7ccccc7)nc56)c4)c3)n2)cc1]

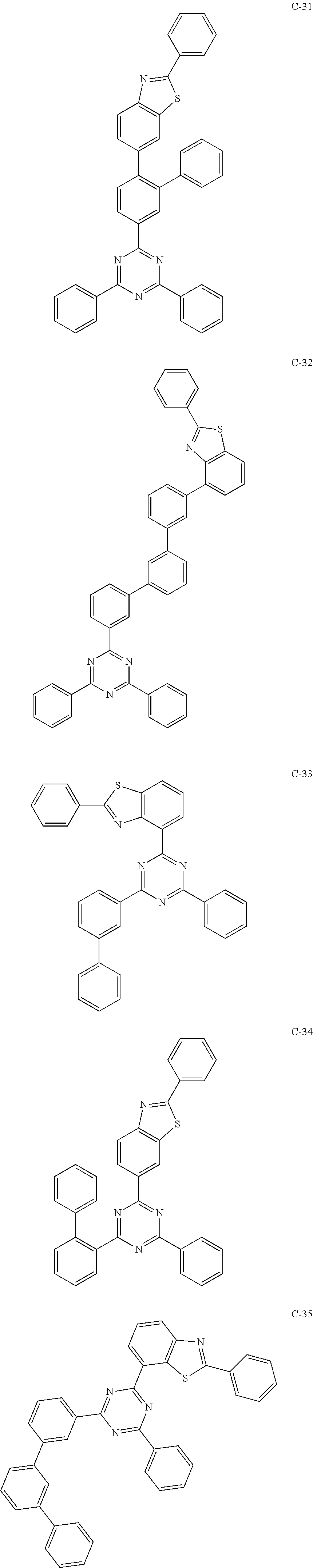 [SMILES: c1ccc(-c2nc(-c3ccccc3)nc(-c3ccc(-c4ccc5nc(-c6ccccc6)sc5c4)c(-c4ccccc4)c3)n2)cc1 ; c1ccc(-c2nc(-c3ccccc3)nc(-c3cccc(-c4cccc(-c5cccc(-c6cccc7sc(-c8ccccc8)nc67)c5)c4)c3)n2)cc1 ; c1ccc(-c2cccc(-c3nc(-c4ccccc4)nc(-c4cccc5sc(-c6ccccc6)nc45)n3)c2)cc1 ; c1ccc(-c2nc(-c3ccc4nc(-c5ccccc5)sc4c3)nc(-c3ccccc3-c3ccccc3)n2)cc1 ; c1ccc(-c2cccc(-c3cccc(-c4nc(-c5ccccc5)nc(-c5cccc6nc(-c7ccccc7)sc56)n4)c3)c2)cc1]

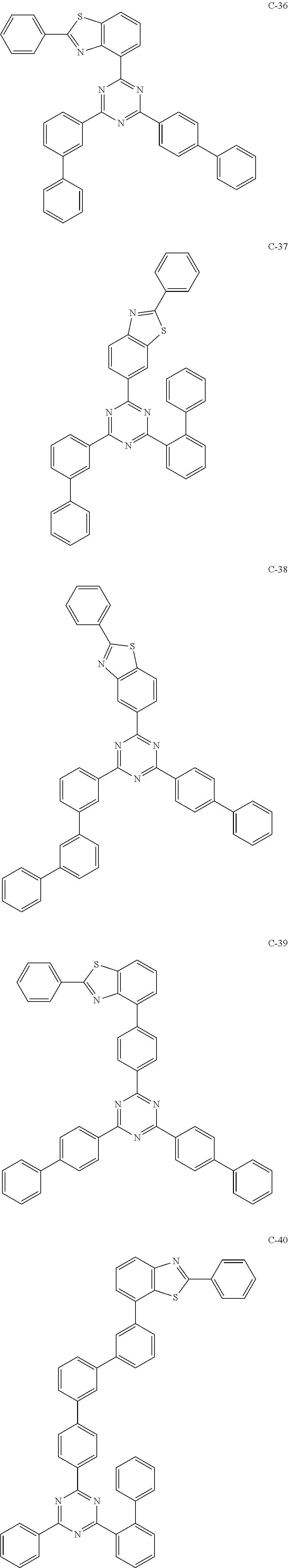 [SMILES: c1ccc(-c2ccc(-c3nc(-c4cccc(-c5ccccc5)c4)nc(-c4cccc5sc(-c6ccccc6)nc45)n3)cc2)cc1 ; c1ccc(-c2cccc(-c3nc(-c4ccc5nc(-c6ccccc6)sc5c4)nc(-c4ccccc4-c4ccccc4)n3)c2)cc1 ; c1ccc(-c2ccc(-c3nc(-c4cccc(-c5cccc(-c6ccccc6)c5)c4)nc(-c4ccc5sc(-c6ccccc6)nc5c4)n3)cc2)cc1 ; c1ccc(-c2ccc(-c3nc(-c4ccc(-c5ccccc5)cc4)nc(-c4ccc(-c5cccc6sc(-c7ccccc7)nc56)cc4)n3)cc2)cc1 ; c1ccc(-c2nc(-c3ccc(-c4cccc(-c5cccc(-c6cccc7nc(-c8ccccc8)sc67)c5)c4)cc3)nc(-c3ccccc3-c3ccccc3)n2)cc1]

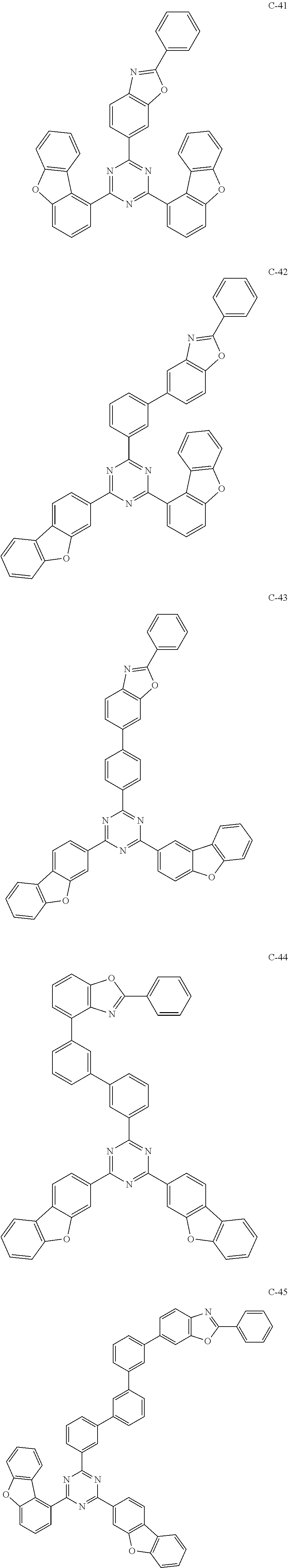 [SMILES: c1ccc(-c2nc3ccc(-c4nc(-c5cccc6oc7ccccc7c56)nc(-c5cccc6oc7ccccc7c56)n4)cc3o2)cc1 ; c1ccc(-c2nc3cc(-c4cccc(-c5nc(-c6ccc7c(c6)oc6ccccc67)nc(-c6cccc7oc8ccccc8c67)n5)c4)ccc3o2)cc1 ; c1ccc(-c2nc3ccc(-c4ccc(-c5nc(-c6ccc7c(c6)oc6ccccc67)nc(-c6ccc7oc8ccccc8c7c6)n5)cc4)cc3o2)cc1 ; c1ccc(-c2nc3c(-c4cccc(-c5cccc(-c6nc(-c7ccc8c(c7)oc7ccccc78)nc(-c7ccc8c(c7)oc7ccccc78)n6)c5)c4)cccc3o2)cc1 ; c1ccc(-c2nc3ccc(-c4cccc(-c5cccc(-c6cccc(-c7nc(-c8ccc9c(c8)oc8ccccc89)nc(-c8cccc9oc%10ccccc%10c89)n7)c6)c5)c4)cc3o2)cc1]

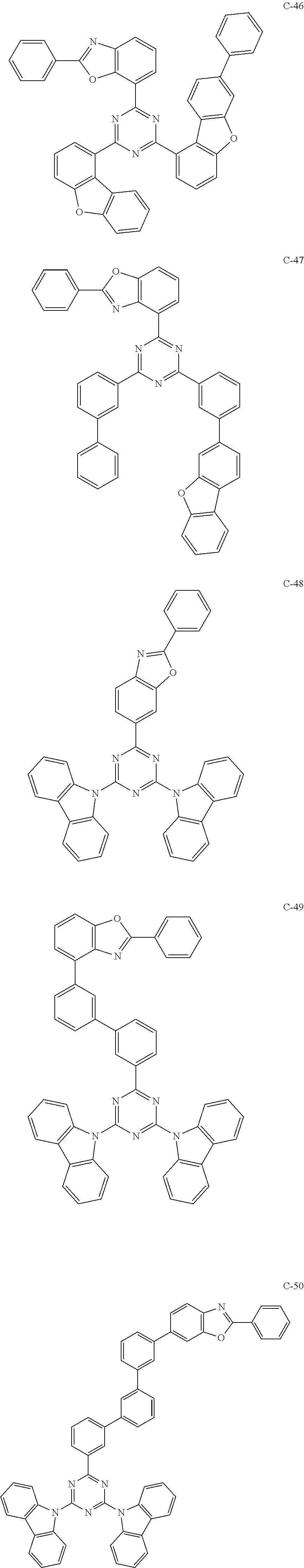 [SMILES: c1ccc(-c2ccc3c(c2)oc2cccc(-c4nc(-c5cccc6nc(-c7ccccc7)oc56)nc(-c5cccc6oc7ccccc7c56)n4)c23)cc1 ; c1ccc(-c2cccc(-c3nc(-c4cccc(-c5ccc6c(c5)oc5ccccc56)c4)nc(-c4cccc5oc(-c6ccccc6)nc45)n3)c2)cc1 ; c1ccc(-c2nc3ccc(-c4nc(-n5c6ccccc6c6ccccc65)nc(-n5c6ccccc6c6ccccc65)n4)cc3o2)cc1 ; c1ccc(-c2nc3c(-c4cccc(-c5cccc(-c6nc(-n7c8ccccc8c8ccccc87)nc(-n7c8ccccc8c8ccccc87)n6)c5)c4)cccc3o2)cc1 ; c1ccc(-c2nc3ccc(-c4cccc(-c5cccc(-c6cccc(-c7nc(-n8c9ccccc9c9ccccc98)nc(-n8c9ccccc9c9ccccc98)n7)c6)c5)c4)cc3o2)cc1]

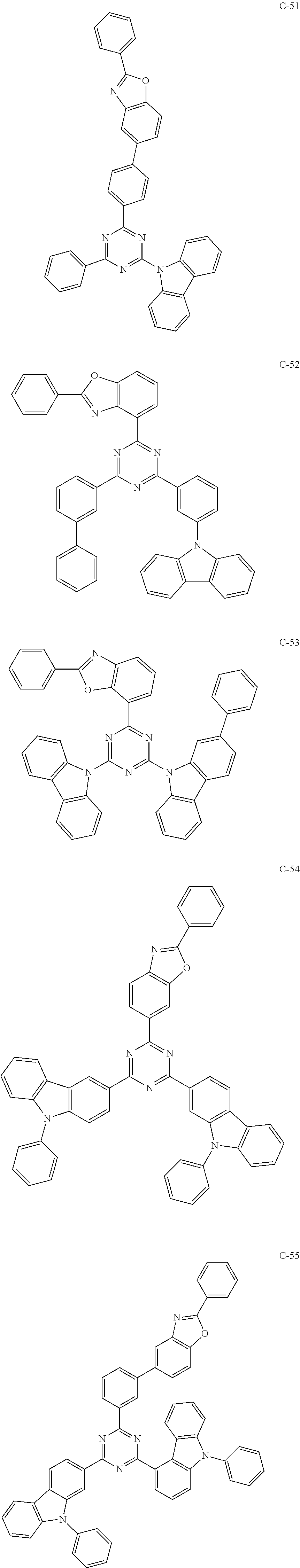 [SMILES: c1ccc(-c2nc(-c3ccc(-c4ccc5oc(-c6ccccc6)nc5c4)cc3)nc(-n3c4ccccc4c4ccccc43)n2)cc1 ; c1ccc(-c2cccc(-c3nc(-c4cccc(-n5c6ccccc6c6ccccc65)c4)nc(-c4cccc5oc(-c6ccccc6)nc45)n3)c2)cc1 ; c1ccc(-c2ccc3c4ccccc4n(-c4nc(-c5cccc6nc(-c7ccccc7)oc56)nc(-n5c6ccccc6c6ccccc65)n4)c3c2)cc1 ; c1ccc(-c2nc3ccc(-c4nc(-c5ccc6c(c5)c5ccccc5n6-c5ccccc5)nc(-c5ccc6c7ccccc7n(-c7ccccc7)c6c5)n4)cc3o2)cc1 ; c1ccc(-c2nc3cc(-c4cccc(-c5nc(-c6ccc7c8ccccc8n(-c8ccccc8)c7c6)nc(-c6cccc7c6c6ccccc6n7-c6ccccc6)n5)c4)ccc3o2)cc1]

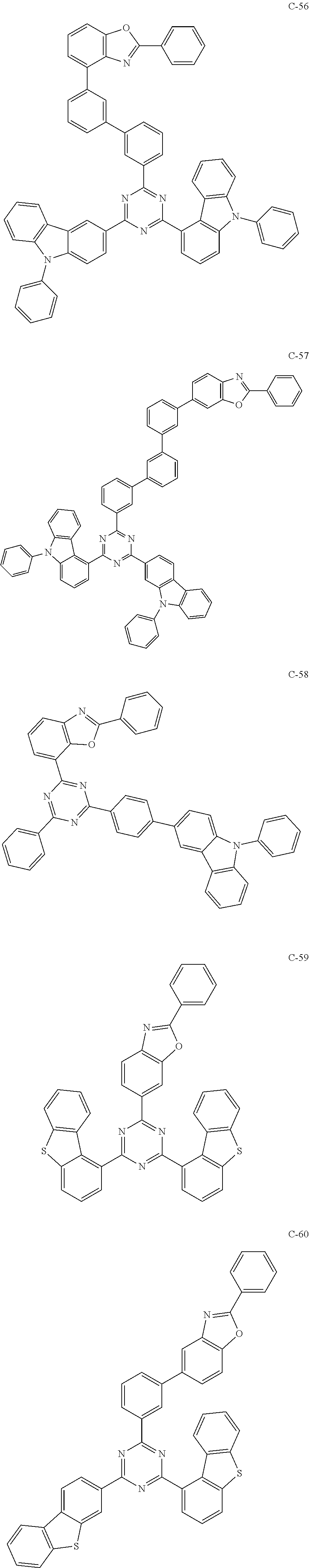

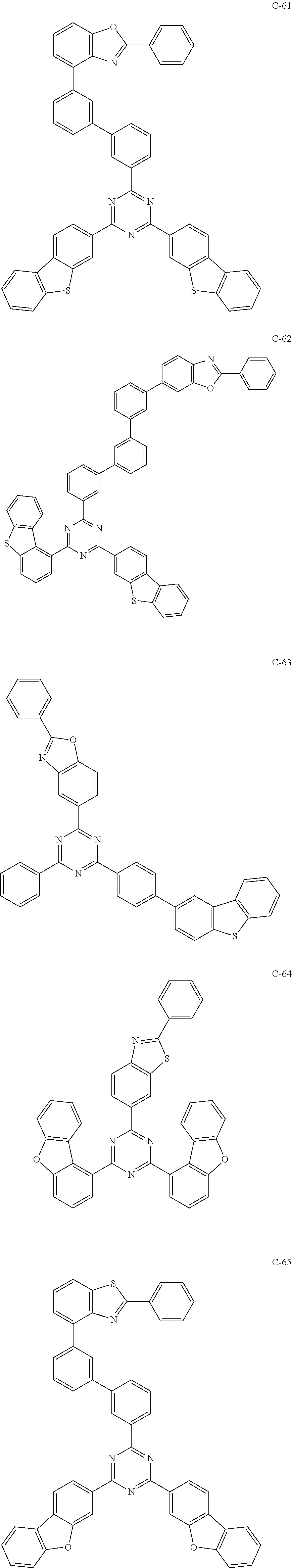 [SMILES: c1ccc(-c2nc3c(-c4cccc(-c5cccc(-c6nc(-c7ccc8c(c7)sc7ccccc78)nc(-c7ccc8c(c7)sc7ccccc78)n6)c5)c4)cccc3o2)cc1 ; c1ccc(-c2nc3ccc(-c4cccc(-c5cccc(-c6cccc(-c7nc(-c8ccc9c(c8)sc8ccccc89)nc(-c8cccc9sc%10ccccc%10c89)n7)c6)c5)c4)cc3o2)cc1 ; c1ccc(-c2nc(-c3ccc(-c4ccc5sc6ccccc6c5c4)cc3)nc(-c3ccc4oc(-c5ccccc5)nc4c3)n2)cc1 ; c1ccc(-c2nc3ccc(-c4nc(-c5cccc6oc7ccccc7c56)nc(-c5cccc6oc7ccccc7c56)n4)cc3s2)cc1 ; c1ccc(-c2nc3c(-c4cccc(-c5cccc(-c6nc(-c7ccc8c(c7)oc7ccccc78)nc(-c7ccc8c(c7)oc7ccccc78)n6)c5)c4)cccc3s2)cc1]

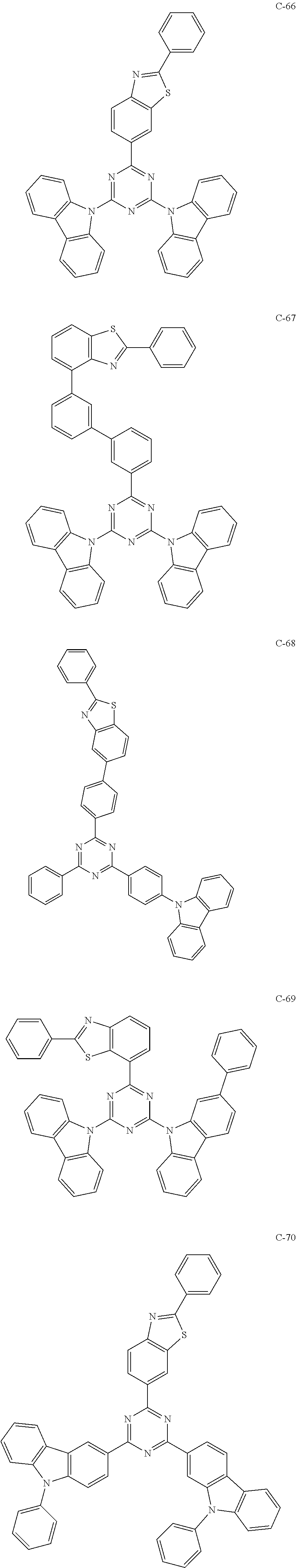 [SMILES: c1ccc(-c2nc3ccc(-c4nc(-n5c6ccccc6c6ccccc65)nc(-n5c6ccccc6c6ccccc65)n4)cc3s2)cc1 ; c1ccc(-c2nc3c(-c4cccc(-c5cccc(-c6nc(-n7c8ccccc8c8ccccc87)nc(-n7c8ccccc8c8ccccc87)n6)c5)c4)cccc3s2)cc1 ; c1ccc(-c2nc(-c3ccc(-c4ccc5sc(-c6ccccc6)nc5c4)cc3)nc(-c3ccc(-n4c5ccccc5c5ccccc54)cc3)n2)cc1 ; c1ccc(-c2ccc3c4ccccc4n(-c4nc(-c5cccc6nc(-c7ccccc7)sc56)nc(-n5c6ccccc6c6ccccc65)n4)c3c2)cc1 ; c1ccc(-c2nc3ccc(-c4nc(-c5ccc6c(c5)c5ccccc5n6-c5ccccc5)nc(-c5ccc6c7ccccc7n(-c7ccccc7)c6c5)n4)cc3s2)cc1]

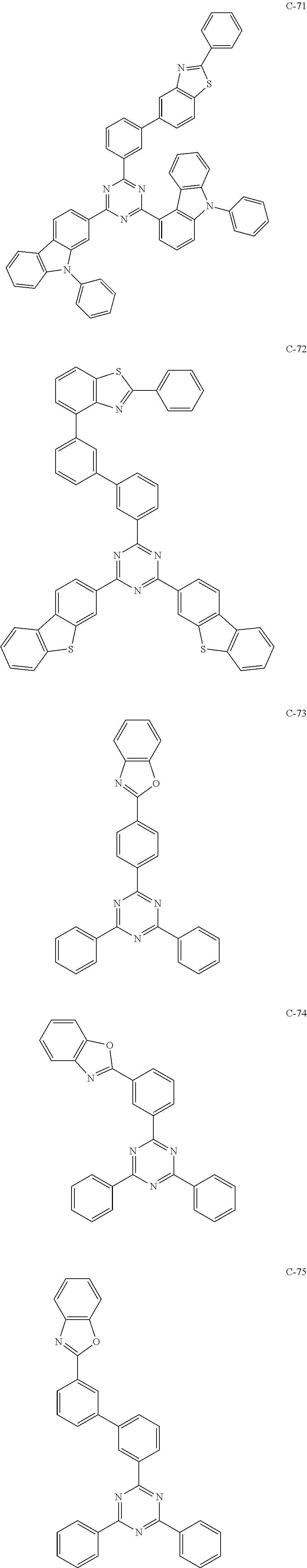 [SMILES: c1ccc(-c2nc3cc(-c4cccc(-c5nc(-c6ccc7c8ccccc8n(-c8ccccc8)c7c6)nc(-c6cccc7c6c6ccccc6n7-c6ccccc6)n5)c4)ccc3s2)cc1 ; c1ccc(-c2nc3c(-c4cccc(-c5cccc(-c6nc(-c7ccc8c(c7)sc7ccccc78)nc(-c7ccc8c(c7)sc7ccccc78)n6)c5)c4)cccc3s2)cc1 ; c1ccc(-c2nc(-c3ccccc3)nc(-c3ccc(-c4nc5ccccc5o4)cc3)n2)cc1 ; c1ccc(-c2nc(-c3ccccc3)nc(-c3cccc(-c4nc5ccccc5o4)c3)n2)cc1 ; c1ccc(-c2nc(-c3ccccc3)nc(-c3cccc(-c4cccc(-c5nc6ccccc6o5)c4)c3)n2)cc1]

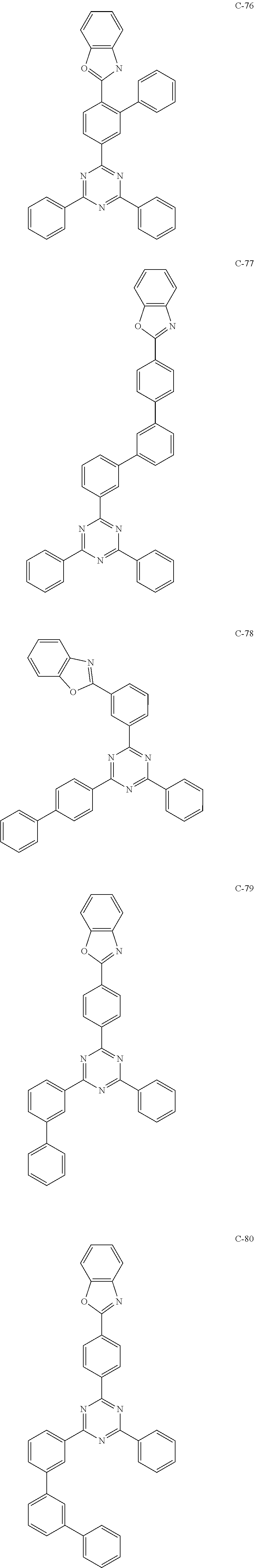

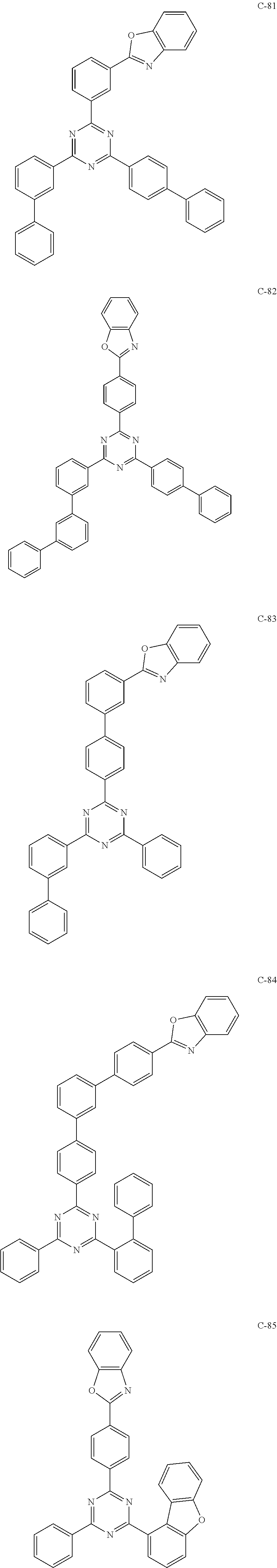 [SMILES: c1ccc(-c2ccc(-c3nc(-c4cccc(-c5ccccc5)c4)nc(-c4cccc(-c5nc6ccccc6o5)c4)n3)cc2)cc1 ; c1ccc(-c2ccc(-c3nc(-c4ccc(-c5nc6ccccc6o5)cc4)nc(-c4cccc(-c5cccc(-c6ccccc6)c5)c4)n3)cc2)cc1 ; c1ccc(-c2cccc(-c3nc(-c4ccccc4)nc(-c4ccc(-c5cccc(-c6nc7ccccc7o6)c5)cc4)n3)c2)cc1 ; c1ccc(-c2nc(-c3ccc(-c4cccc(-c5ccc(-c6nc7ccccc7o6)cc5)c4)cc3)nc(-c3ccccc3-c3ccccc3)n2)cc1 ; c1ccc(-c2nc(-c3ccc(-c4nc5ccccc5o4)cc3)nc(-c3cccc4oc5ccccc5c34)n2)cc1]

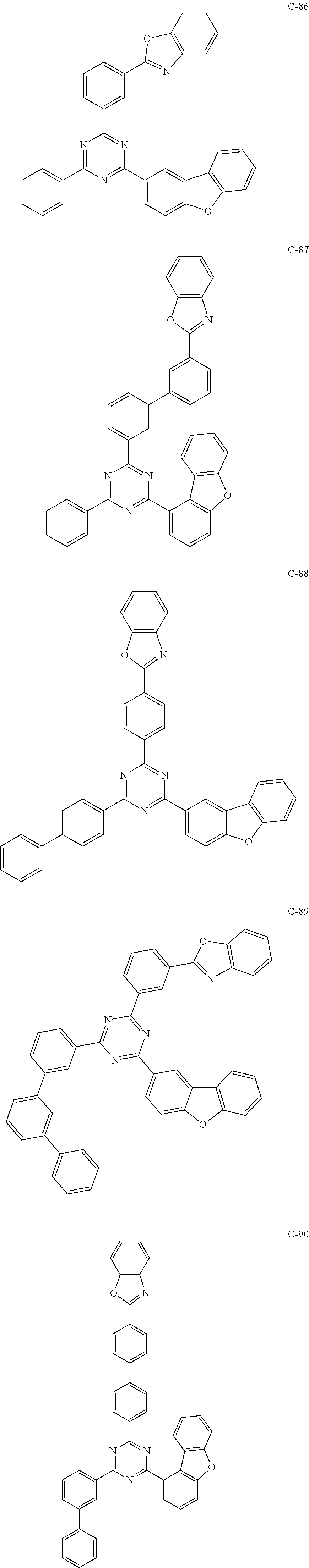 [SMILES: c1ccc(-c2nc(-c3cccc(-c4nc5ccccc5o4)c3)nc(-c3ccc4oc5ccccc5c4c3)n2)cc1 ; c1ccc(-c2nc(-c3cccc(-c4cccc(-c5nc6ccccc6o5)c4)c3)nc(-c3cccc4oc5ccccc5c34)n2)cc1 ; c1ccc(-c2ccc(-c3nc(-c4ccc(-c5nc6ccccc6o5)cc4)nc(-c4ccc5oc6ccccc6c5c4)n3)cc2)cc1 ; c1ccc(-c2cccc(-c3cccc(-c4nc(-c5cccc(-c6nc7ccccc7o6)c5)nc(-c5ccc6oc7ccccc7c6c5)n4)c3)c2)cc1 ; c1ccc(-c2cccc(-c3nc(-c4ccc(-c5ccc(-c6nc7ccccc7o6)cc5)cc4)nc(-c4cccc5oc6ccccc6c45)n3)c2)cc1]

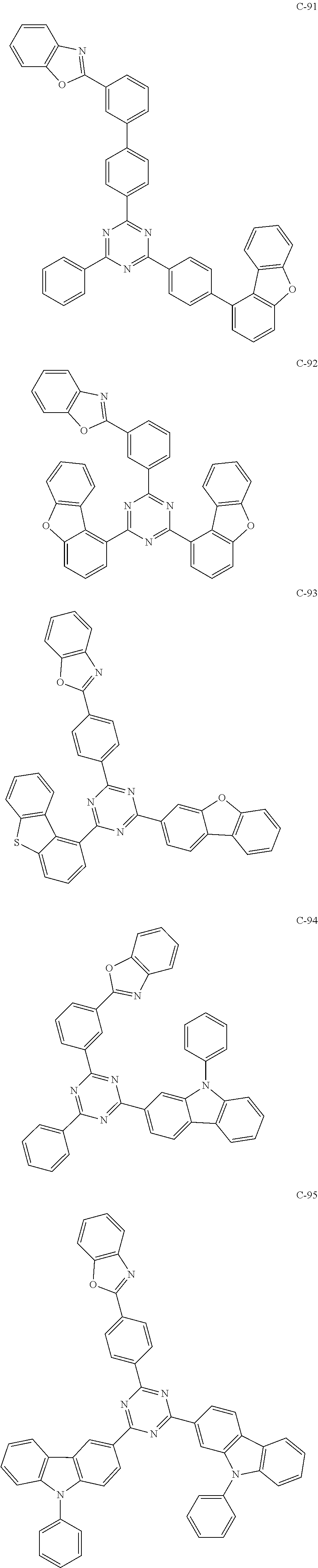 [SMILES: c1ccc(-c2nc(-c3ccc(-c4cccc(-c5nc6ccccc6o5)c4)cc3)nc(-c3ccc(-c4cccc5oc6ccccc6c45)cc3)n2)cc1 ; c1cc(-c2nc(-c3cccc4oc5ccccc5c34)nc(-c3cccc4oc5ccccc5c34)n2)cc(-c2nc3ccccc3o2)c1 ; c1ccc2oc(-c3ccc(-c4nc(-c5ccc6c(c5)oc5ccccc56)nc(-c5cccc6sc7ccccc7c56)n4)cc3)nc2c1 ; c1ccc(-c2nc(-c3cccc(-c4nc5ccccc5o4)c3)nc(-c3ccc4c5ccccc5n(-c5ccccc5)c4c3)n2)cc1 ; c1ccc(-n2c3ccccc3c3cc(-c4nc(-c5ccc(-c6nc7ccccc7o6)cc5)nc(-c5ccc6c7ccccc7n(-c7ccccc7)c6c5)n4)ccc32)cc1]

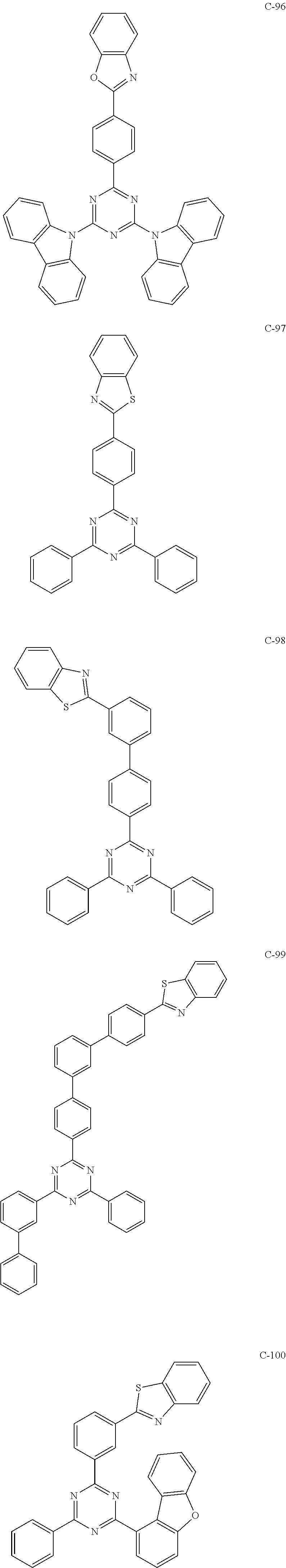 [SMILES: c1ccc2oc(-c3ccc(-c4nc(-n5c6ccccc6c6ccccc65)nc(-n5c6ccccc6c6ccccc65)n4)cc3)nc2c1 ; c1ccc(-c2nc(-c3ccccc3)nc(-c3ccc(-c4nc5ccccc5s4)cc3)n2)cc1 ; c1ccc(-c2nc(-c3ccccc3)nc(-c3ccc(-c4cccc(-c5nc6ccccc6s5)c4)cc3)n2)cc1 ; c1ccc(-c2cccc(-c3nc(-c4ccccc4)nc(-c4ccc(-c5cccc(-c6ccc(-c7nc8ccccc8s7)cc6)c5)cc4)n3)c2)cc1 ; c1ccc(-c2nc(-c3cccc(-c4nc5ccccc5s4)c3)nc(-c3cccc4oc5ccccc5c34)n2)cc1]

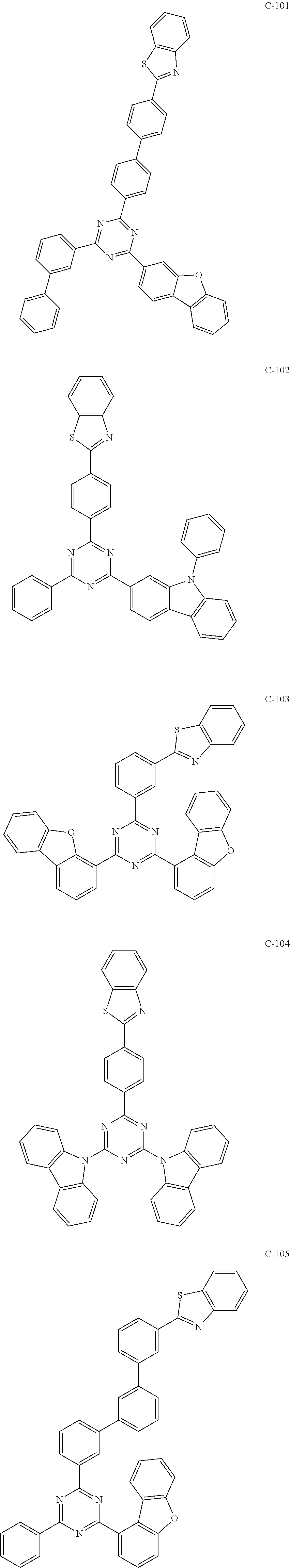 [SMILES: c1ccc(-c2cccc(-c3nc(-c4ccc(-c5ccc(-c6nc7ccccc7s6)cc5)cc4)nc(-c4ccc5c(c4)oc4ccccc45)n3)c2)cc1 ; c1ccc(-c2nc(-c3ccc(-c4nc5ccccc5s4)cc3)nc(-c3ccc4c5ccccc5n(-c5ccccc5)c4c3)n2)cc1 ; c1cc(-c2nc(-c3cccc4c3oc3ccccc34)nc(-c3cccc4oc5ccccc5c34)n2)cc(-c2nc3ccccc3s2)c1 ; c1ccc2sc(-c3ccc(-c4nc(-n5c6ccccc6c6ccccc65)nc(-n5c6ccccc6c6ccccc65)n4)cc3)nc2c1 ; c1ccc(-c2nc(-c3cccc(-c4cccc(-c5cccc(-c6nc7ccccc7s6)c5)c4)c3)nc(-c3cccc4oc5ccccc5c34)n2)cc1]

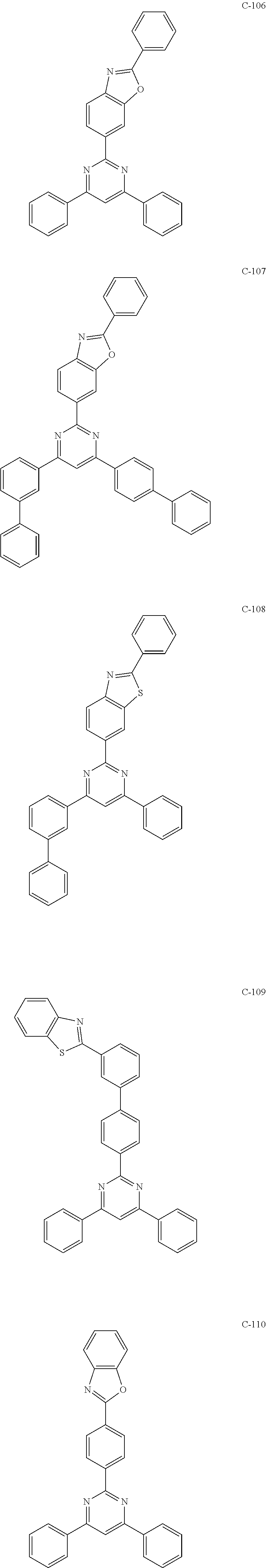 [SMILES: c1ccc(-c2cc(-c3ccccc3)nc(-c3ccc4nc(-c5ccccc5)oc4c3)n2)cc1 ; c1ccc(-c2ccc(-c3cc(-c4cccc(-c5ccccc5)c4)nc(-c4ccc5nc(-c6ccccc6)oc5c4)n3)cc2)cc1 ; c1ccc(-c2cccc(-c3cc(-c4ccccc4)nc(-c4ccc5nc(-c6ccccc6)sc5c4)n3)c2)cc1 ; c1ccc(-c2cc(-c3ccccc3)nc(-c3ccc(-c4cccc(-c5nc6ccccc6s5)c4)cc3)n2)cc1 ; c1ccc(-c2cc(-c3ccccc3)nc(-c3ccc(-c4nc5ccccc5o4)cc3)n2)cc1]

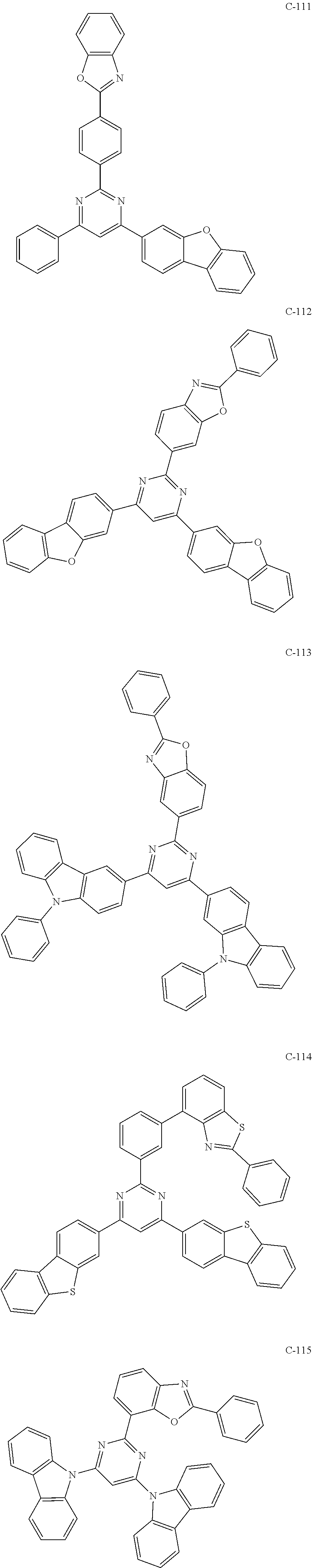 [SMILES: c1ccc(-c2cc(-c3ccc4c(c3)oc3ccccc34)nc(-c3ccc(-c4nc5ccccc5o4)cc3)n2)cc1 ; c1ccc(-c2nc3ccc(-c4nc(-c5ccc6c(c5)oc5ccccc56)cc(-c5ccc6c(c5)oc5ccccc56)n4)cc3o2)cc1 ; c1ccc(-c2nc3cc(-c4nc(-c5ccc6c(c5)c5ccccc5n6-c5ccccc5)cc(-c5ccc6c7ccccc7n(-c7ccccc7)c6c5)n4)ccc3o2)cc1 ; c1ccc(-c2nc3c(-c4cccc(-c5nc(-c6ccc7c(c6)sc6ccccc67)cc(-c6ccc7c(c6)sc6ccccc67)n5)c4)cccc3s2)cc1 ; c1ccc(-c2nc3cccc(-c4nc(-n5c6ccccc6c6ccccc65)cc(-n5c6ccccc6c6ccccc65)n4)c3o2)cc1]

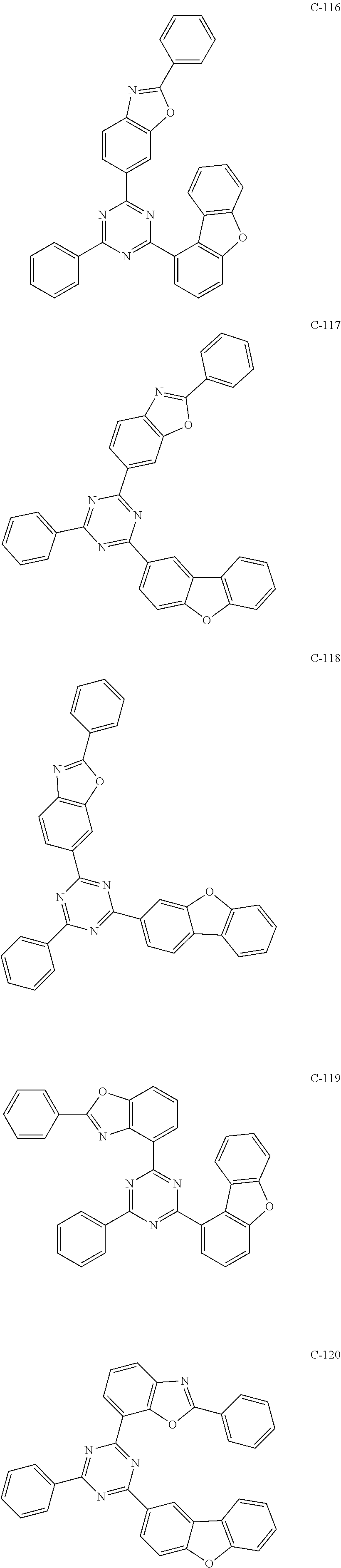 [SMILES: c1ccc(-c2nc(-c3ccc4nc(-c5ccccc5)oc4c3)nc(-c3cccc4oc5ccccc5c34)n2)cc1 ; c1ccc(-c2nc(-c3ccc4nc(-c5ccccc5)oc4c3)nc(-c3ccc4oc5ccccc5c4c3)n2)cc1 ; c1ccc(-c2nc(-c3ccc4nc(-c5ccccc5)oc4c3)nc(-c3ccc4c(c3)oc3ccccc34)n2)cc1 ; c1ccc(-c2nc(-c3cccc4oc(-c5ccccc5)nc34)nc(-c3cccc4oc5ccccc5c34)n2)cc1 ; c1ccc(-c2nc(-c3ccc4oc5ccccc5c4c3)nc(-c3cccc4nc(-c5ccccc5)oc34)n2)cc1]

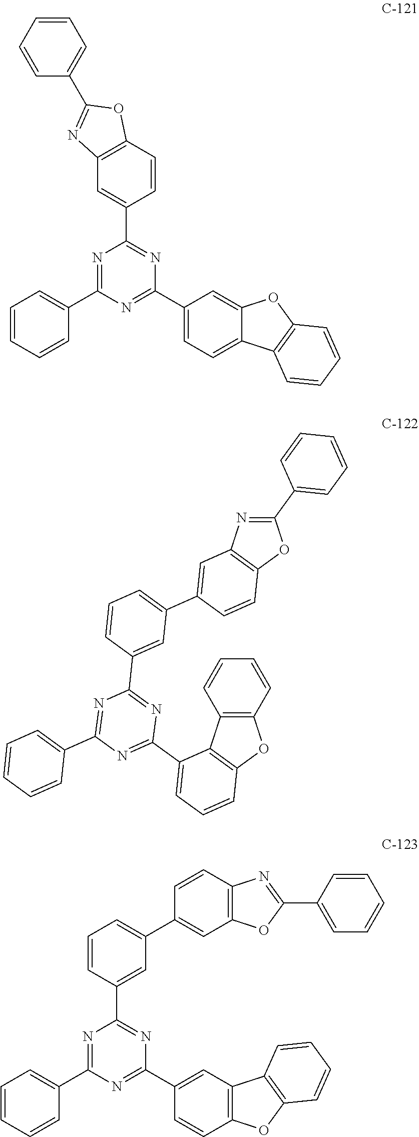 [SMILES: c1ccc(-c2nc(-c3ccc4oc(-c5ccccc5)nc4c3)nc(-c3ccc4c(c3)oc3ccccc34)n2)cc1 ; c1ccc(-c2nc(-c3cccc(-c4ccc5oc(-c6ccccc6)nc5c4)c3)nc(-c3cccc4oc5ccccc5c34)n2)cc1 ; c1ccc(-c2nc(-c3cccc(-c4ccc5nc(-c6ccccc6)oc5c4)c3)nc(-c3ccc4oc5ccccc5c4c3)n2)cc1]

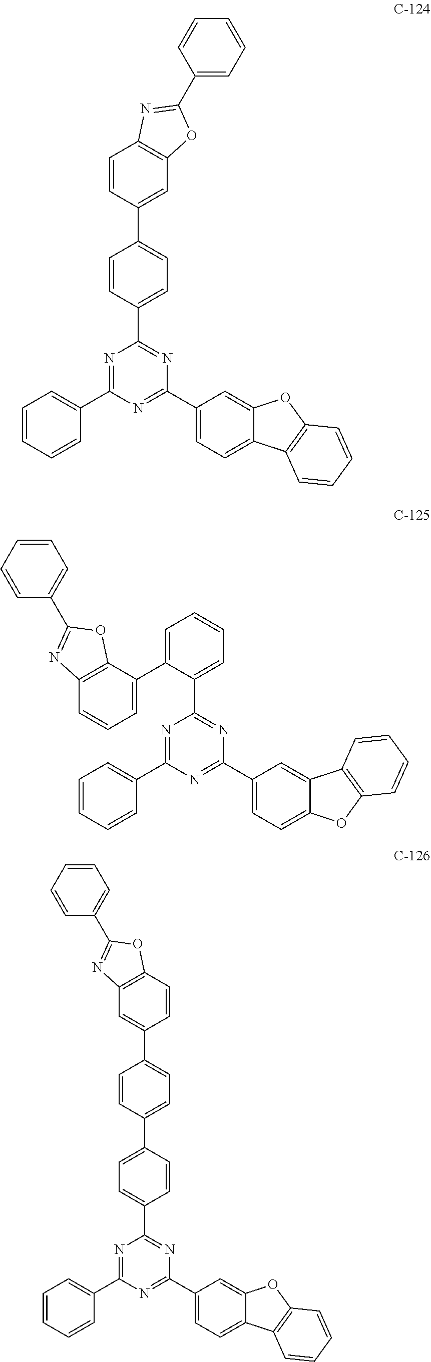

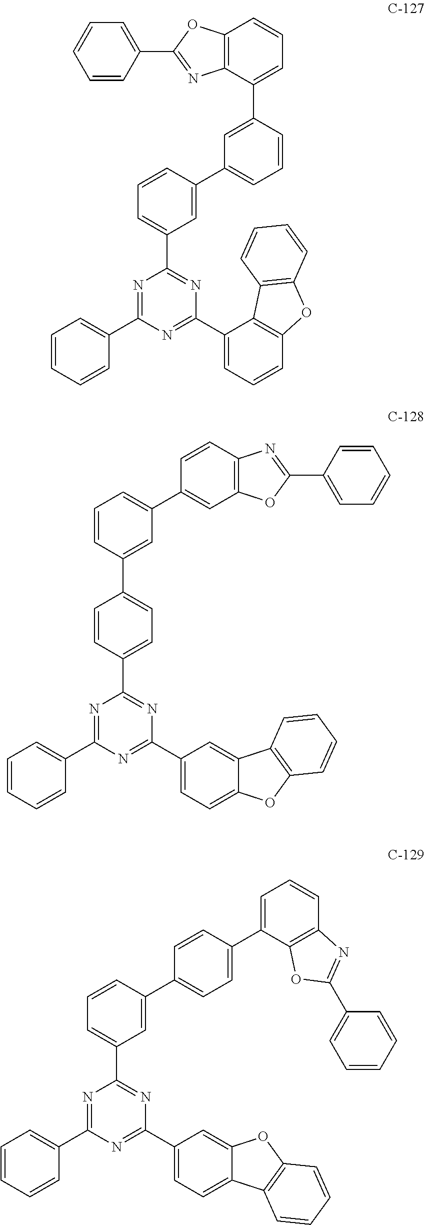 [SMILES: c1ccc(-c2nc(-c3cccc(-c4cccc(-c5cccc6oc(-c7ccccc7)nc56)c4)c3)nc(-c3cccc4oc5ccccc5c34)n2)cc1 ; c1ccc(-c2nc(-c3ccc(-c4cccc(-c5ccc6nc(-c7ccccc7)oc6c5)c4)cc3)nc(-c3ccc4oc5ccccc5c4c3)n2)cc1 ; c1ccc(-c2nc(-c3cccc(-c4ccc(-c5cccc6nc(-c7ccccc7)oc56)cc4)c3)nc(-c3ccc4c(c3)oc3ccccc34)n2)cc1]

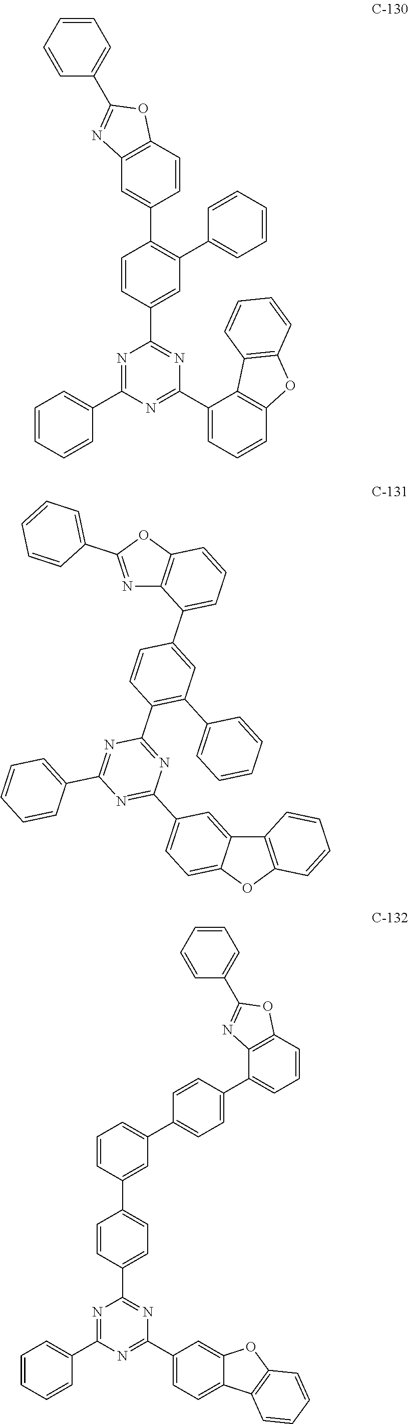

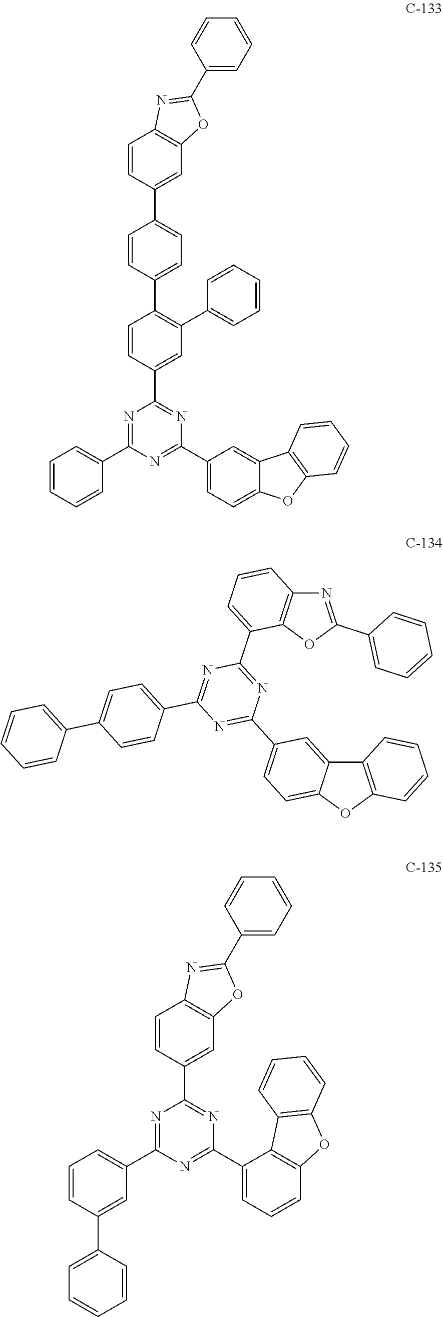 [SMILES: c1ccc(-c2nc(-c3ccc(-c4ccc(-c5ccc6nc(-c7ccccc7)oc6c5)cc4)c(-c4ccccc4)c3)nc(-c3ccc4oc5ccccc5c4c3)n2)cc1 ; c1ccc(-c2ccc(-c3nc(-c4ccc5oc6ccccc6c5c4)nc(-c4cccc5nc(-c6ccccc6)oc45)n3)cc2)cc1 ; c1ccc(-c2cccc(-c3nc(-c4ccc5nc(-c6ccccc6)oc5c4)nc(-c4cccc5oc6ccccc6c45)n3)c2)cc1]

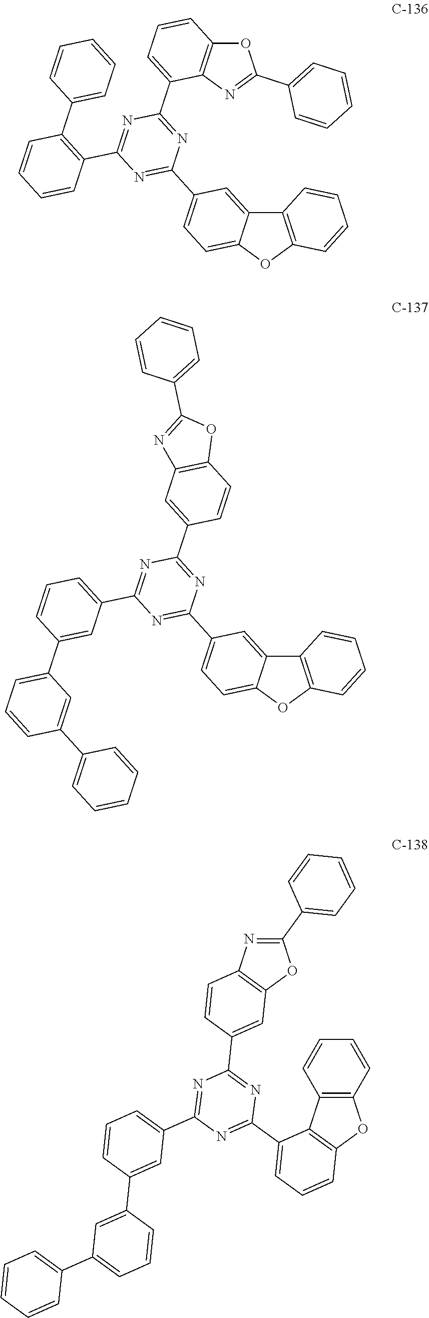 [SMILES: c1ccc(-c2nc3c(-c4nc(-c5ccc6oc7ccccc7c6c5)nc(-c5ccccc5-c5ccccc5)n4)cccc3o2)cc1 ; c1ccc(-c2cccc(-c3cccc(-c4nc(-c5ccc6oc(-c7ccccc7)nc6c5)nc(-c5ccc6oc7ccccc7c6c5)n4)c3)c2)cc1 ; c1ccc(-c2cccc(-c3cccc(-c4nc(-c5ccc6nc(-c7ccccc7)oc6c5)nc(-c5cccc6oc7ccccc7c56)n4)c3)c2)cc1]

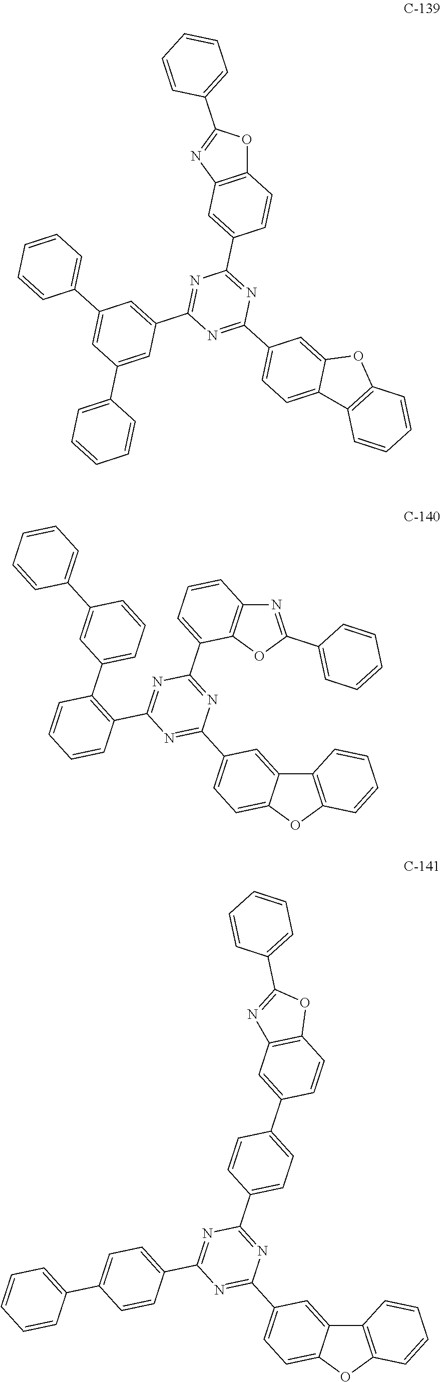 [SMILES: c1ccc(-c2cc(-c3ccccc3)cc(-c3nc(-c4ccc5oc(-c6ccccc6)nc5c4)nc(-c4ccc5c(c4)oc4ccccc45)n3)c2)cc1 ; c1ccc(-c2cccc(-c3ccccc3-c3nc(-c4ccc5oc6ccccc6c5c4)nc(-c4cccc5nc(-c6ccccc6)oc45)n3)c2)cc1 ; c1ccc(-c2ccc(-c3nc(-c4ccc(-c5ccc6oc(-c7ccccc7)nc6c5)cc4)nc(-c4ccc5oc6ccccc6c5c4)n3)cc2)cc1]

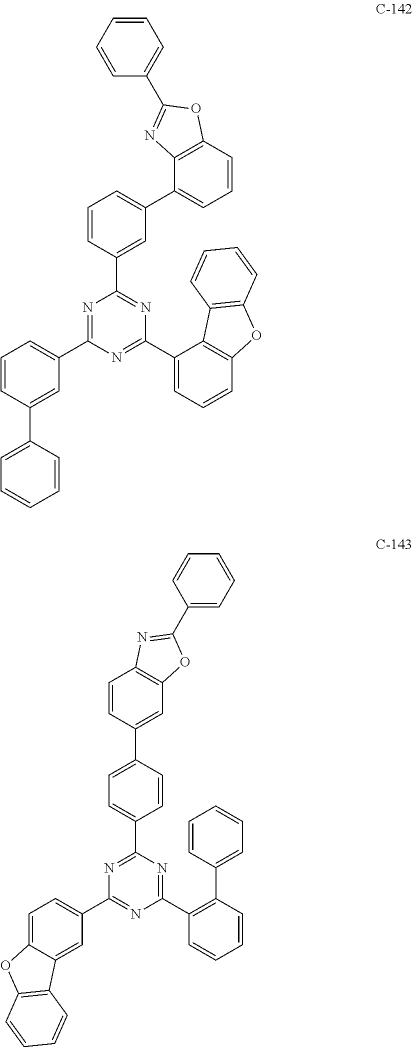 [SMILES: c1ccc(-c2cccc(-c3nc(-c4cccc(-c5cccc6oc(-c7ccccc7)nc56)c4)nc(-c4cccc5oc6ccccc6c45)n3)c2)cc1 ; c1ccc(-c2nc3ccc(-c4ccc(-c5nc(-c6ccc7oc8ccccc8c7c6)nc(-c6ccccc6-c6ccccc6)n5)cc4)cc3o2)cc1]

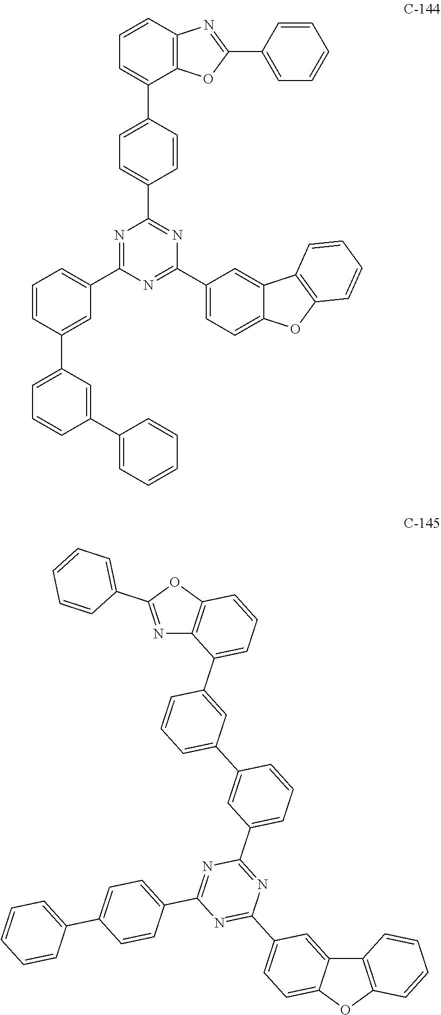 [SMILES: c1ccc(-c2cccc(-c3cccc(-c4nc(-c5ccc(-c6cccc7nc(-c8ccccc8)oc67)cc5)nc(-c5ccc6oc7ccccc7c6c5)n4)c3)c2)cc1 ; c1ccc(-c2ccc(-c3nc(-c4cccc(-c5cccc(-c6cccc7oc(-c8ccccc8)nc67)c5)c4)nc(-c4ccc5oc6ccccc6c5c4)n3)cc2)cc1]

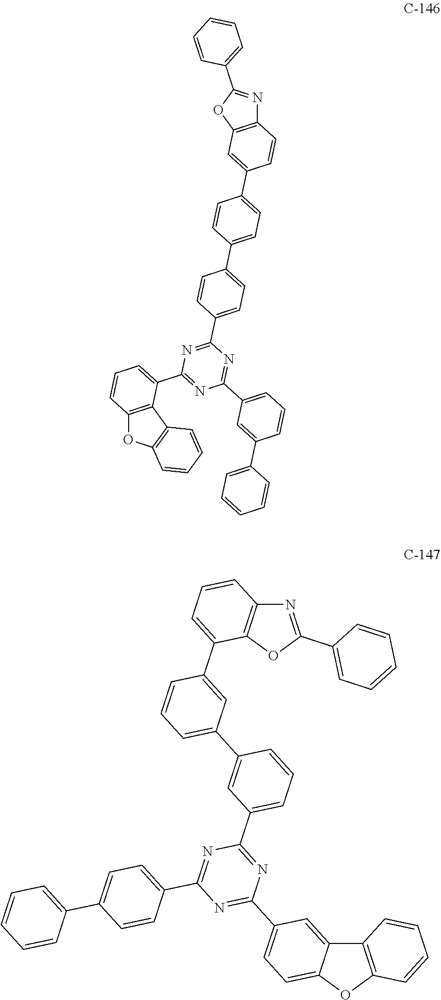 [SMILES: c1ccc(-c2cccc(-c3nc(-c4ccc(-c5ccc(-c6ccc7nc(-c8ccccc8)oc7c6)cc5)cc4)nc(-c4cccc5oc6ccccc6c45)n3)c2)cc1 ; c1ccc(-c2ccc(-c3nc(-c4cccc(-c5cccc(-c6cccc7nc(-c8ccccc8)oc67)c5)c4)nc(-c4ccc5oc6ccccc6c5c4)n3)cc2)cc1]

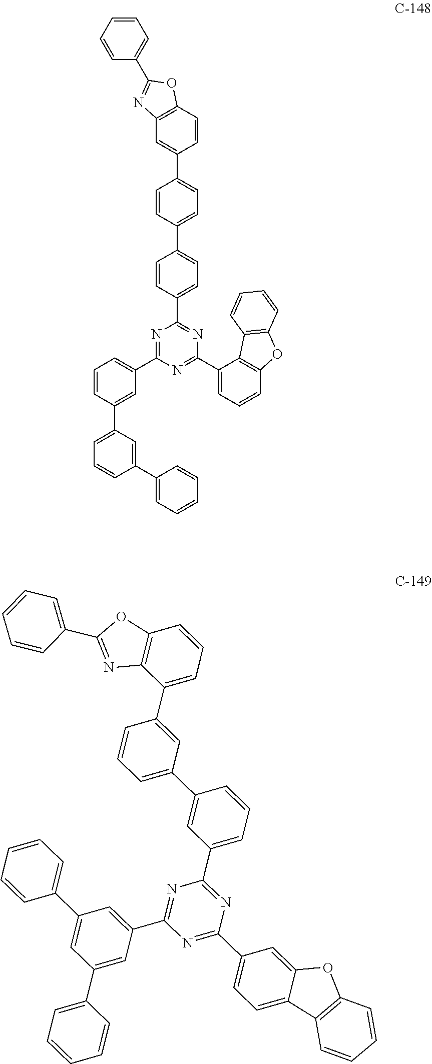 [SMILES: c1ccc(-c2cccc(-c3cccc(-c4nc(-c5ccc(-c6ccc(-c7ccc8oc(-c9ccccc9)nc8c7)cc6)cc5)nc(-c5cccc6oc7ccccc7c56)n4)c3)c2)cc1 ; c1ccc(-c2cc(-c3ccccc3)cc(-c3nc(-c4cccc(-c5cccc(-c6cccc7oc(-c8ccccc8)nc67)c5)c4)nc(-c4ccc5c(c4)oc4ccccc45)n3)c2)cc1]

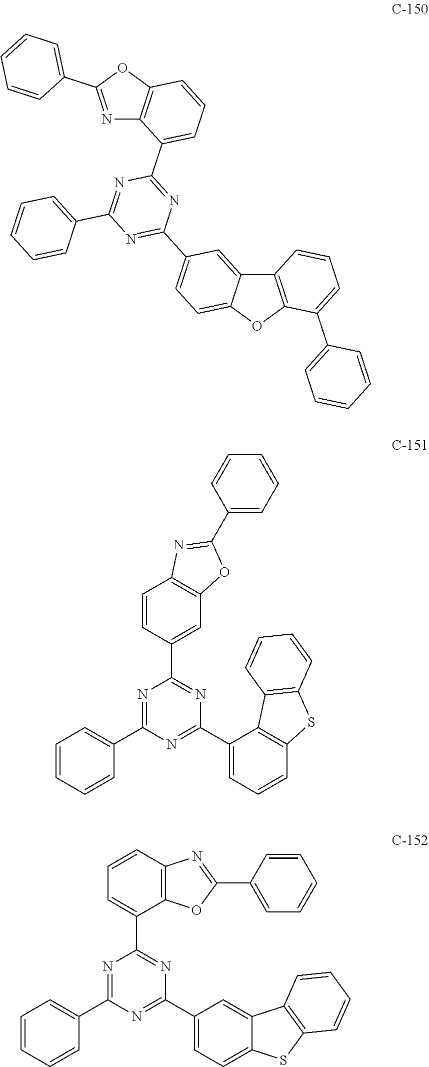 [SMILES: c1ccc(-c2nc(-c3ccc4oc5c(-c6ccccc6)cccc5c4c3)nc(-c3cccc4oc(-c5ccccc5)nc34)n2)cc1 ; c1ccc(-c2nc(-c3ccc4nc(-c5ccccc5)oc4c3)nc(-c3cccc4sc5ccccc5c34)n2)cc1 ; c1ccc(-c2nc(-c3ccc4sc5ccccc5c4c3)nc(-c3cccc4nc(-c5ccccc5)oc34)n2)cc1]

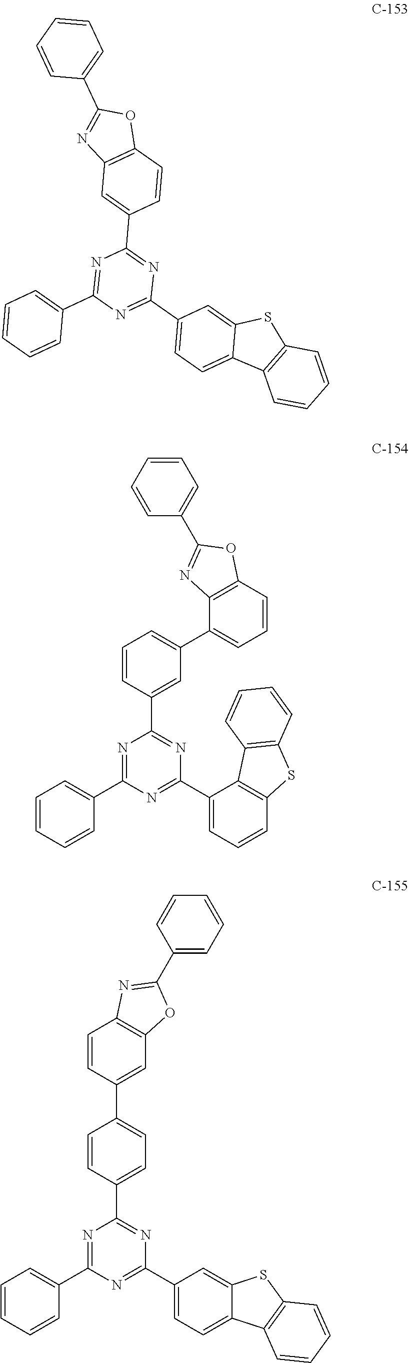 [SMILES: c1ccc(-c2nc(-c3ccc4oc(-c5ccccc5)nc4c3)nc(-c3ccc4c(c3)sc3ccccc34)n2)cc1 ; c1ccc(-c2nc(-c3cccc(-c4cccc5oc(-c6ccccc6)nc45)c3)nc(-c3cccc4sc5ccccc5c34)n2)cc1 ; c1ccc(-c2nc(-c3ccc(-c4ccc5nc(-c6ccccc6)oc5c4)cc3)nc(-c3ccc4c(c3)sc3ccccc34)n2)cc1]

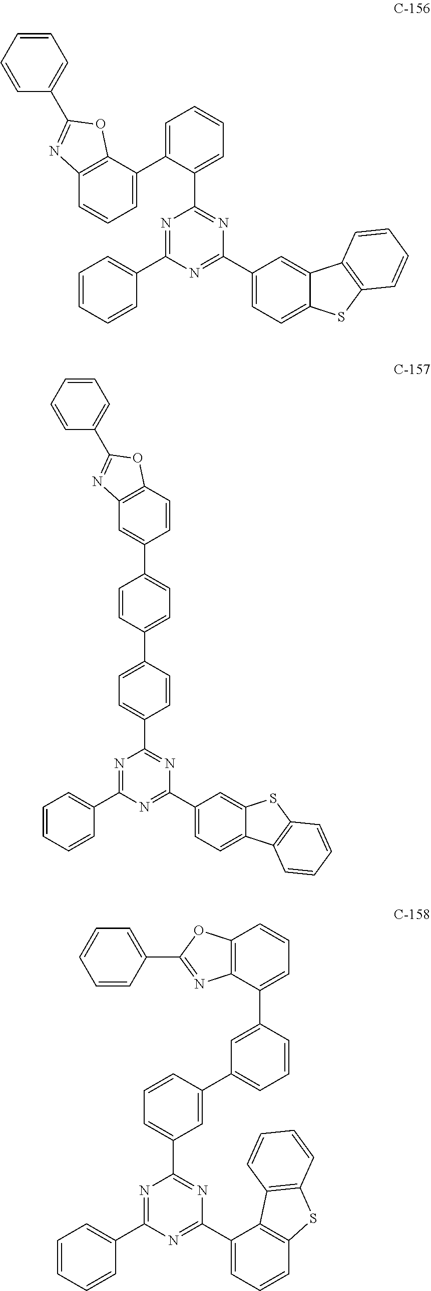 [SMILES: c1ccc(-c2nc(-c3ccc4sc5ccccc5c4c3)nc(-c3ccccc3-c3cccc4nc(-c5ccccc5)oc34)n2)cc1 ; c1ccc(-c2nc(-c3ccc(-c4ccc(-c5ccc6oc(-c7ccccc7)nc6c5)cc4)cc3)nc(-c3ccc4c(c3)sc3ccccc34)n2)cc1 ; c1ccc(-c2nc(-c3cccc(-c4cccc(-c5cccc6oc(-c7ccccc7)nc56)c4)c3)nc(-c3cccc4sc5ccccc5c34)n2)cc1]

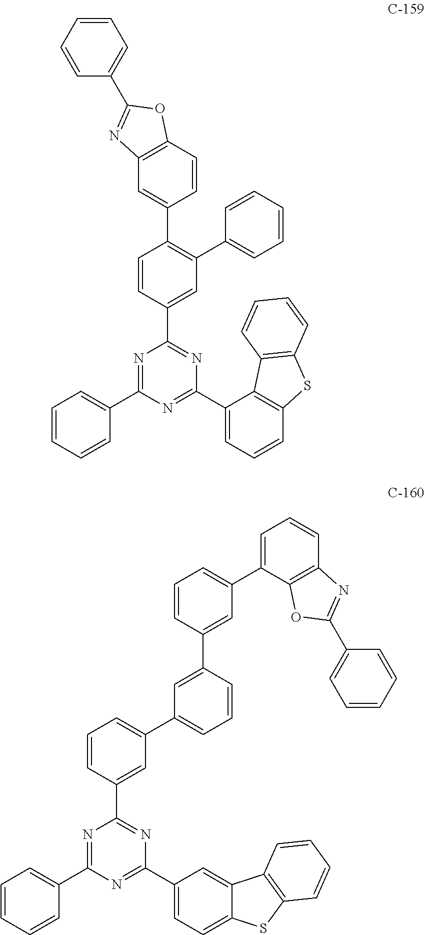 [SMILES: c1ccc(-c2nc(-c3ccc(-c4ccc5oc(-c6ccccc6)nc5c4)c(-c4ccccc4)c3)nc(-c3cccc4sc5ccccc5c34)n2)cc1 ; c1ccc(-c2nc(-c3cccc(-c4cccc(-c5cccc(-c6cccc7nc(-c8ccccc8)oc67)c5)c4)c3)nc(-c3ccc4sc5ccccc5c4c3)n2)cc1]

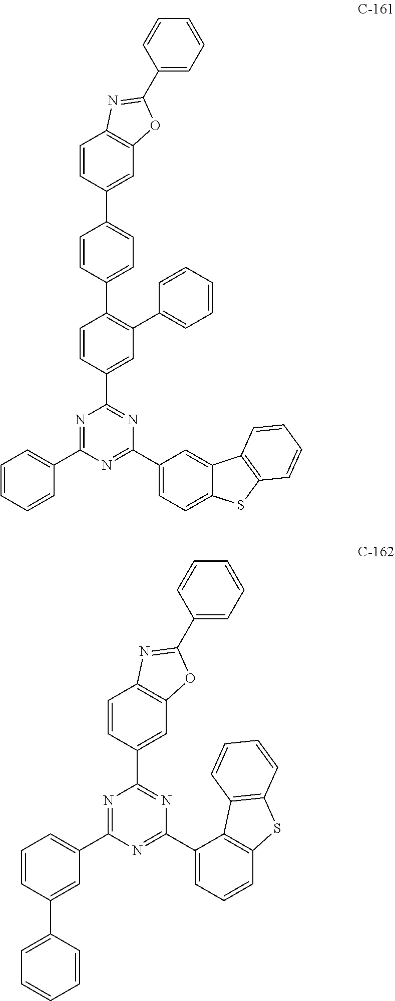 [SMILES: c1ccc(-c2nc(-c3ccc(-c4ccc(-c5ccc6nc(-c7ccccc7)oc6c5)cc4)c(-c4ccccc4)c3)nc(-c3ccc4sc5ccccc5c4c3)n2)cc1 ; c1ccc(-c2cccc(-c3nc(-c4ccc5nc(-c6ccccc6)oc5c4)nc(-c4cccc5sc6ccccc6c45)n3)c2)cc1]

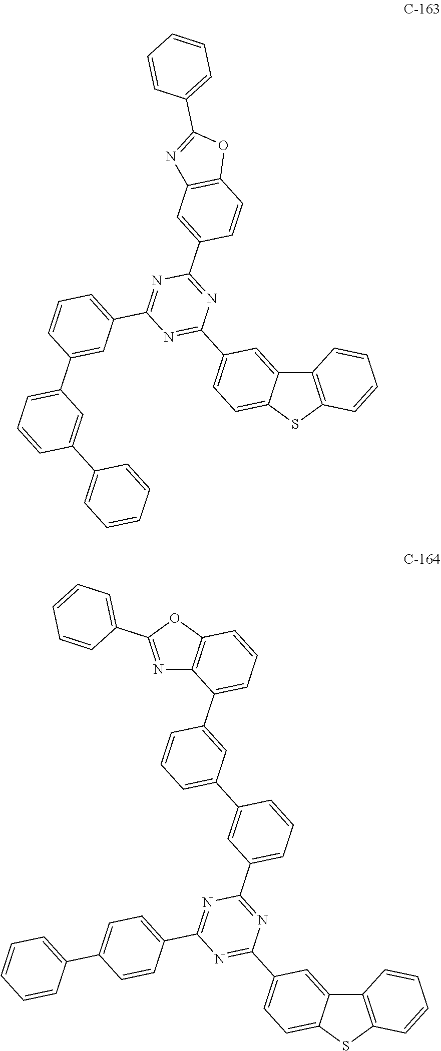 [SMILES: c1ccc(-c2cccc(-c3cccc(-c4nc(-c5ccc6oc(-c7ccccc7)nc6c5)nc(-c5ccc6sc7ccccc7c6c5)n4)c3)c2)cc1 ; c1ccc(-c2ccc(-c3nc(-c4cccc(-c5cccc(-c6cccc7oc(-c8ccccc8)nc67)c5)c4)nc(-c4ccc5sc6ccccc6c5c4)n3)cc2)cc1]

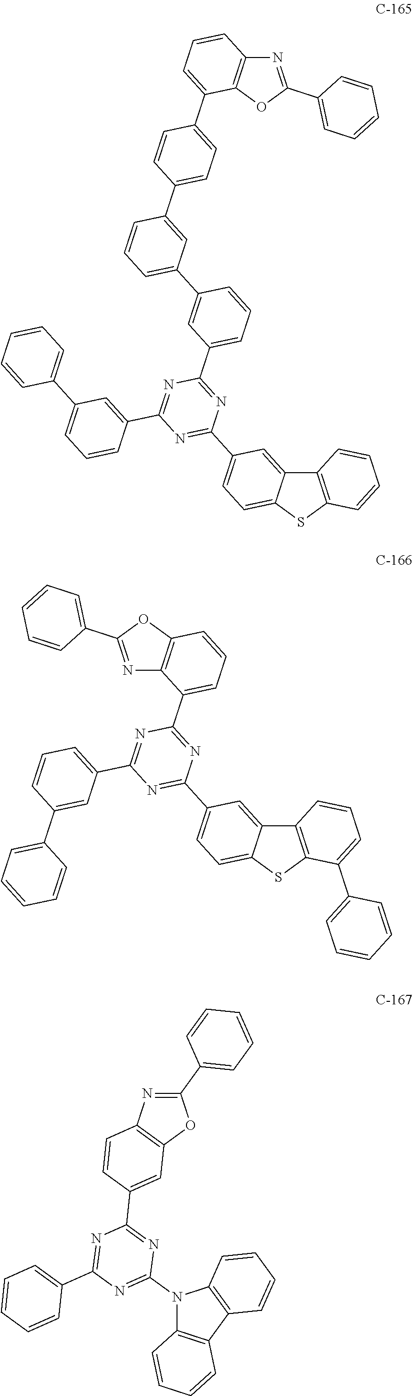 [SMILES: c1ccc(-c2cccc(-c3nc(-c4cccc(-c5cccc(-c6ccc(-c7cccc8nc(-c9ccccc9)oc78)cc6)c5)c4)nc(-c4ccc5sc6ccccc6c5c4)n3)c2)cc1 ; c1ccc(-c2cccc(-c3nc(-c4ccc5sc6c(-c7ccccc7)cccc6c5c4)nc(-c4cccc5oc(-c6ccccc6)nc45)n3)c2)cc1 ; c1ccc(-c2nc(-c3ccc4nc(-c5ccccc5)oc4c3)nc(-n3c4ccccc4c4ccccc43)n2)cc1]

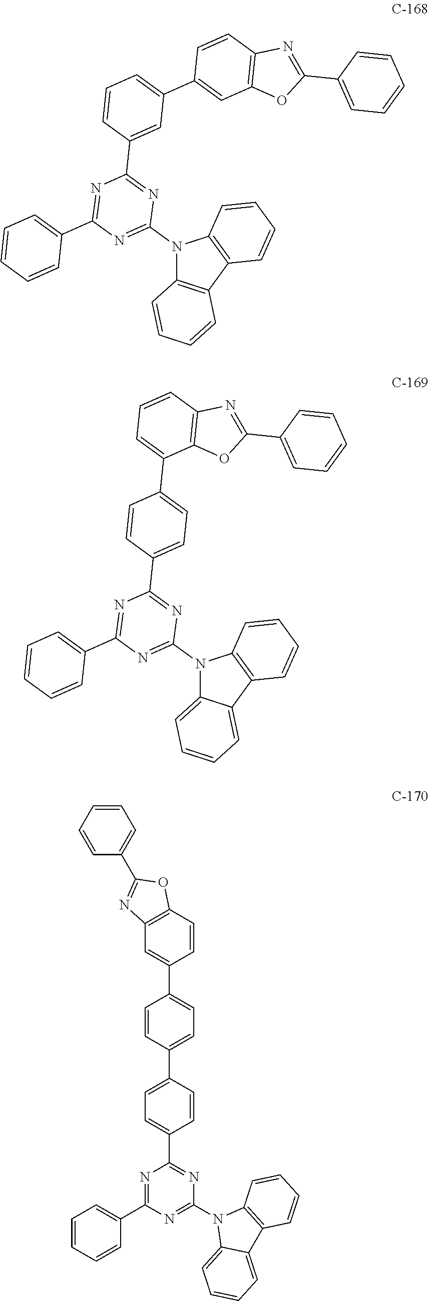 [SMILES: c1ccc(-c2nc(-c3cccc(-c4ccc5nc(-c6ccccc6)oc5c4)c3)nc(-n3c4ccccc4c4ccccc43)n2)cc1 ; c1ccc(-c2nc(-c3ccc(-c4cccc5nc(-c6ccccc6)oc45)cc3)nc(-n3c4ccccc4c4ccccc43)n2)cc1 ; c1ccc(-c2nc(-c3ccc(-c4ccc(-c5ccc6oc(-c7ccccc7)nc6c5)cc4)cc3)nc(-n3c4ccccc4c4ccccc43)n2)cc1]

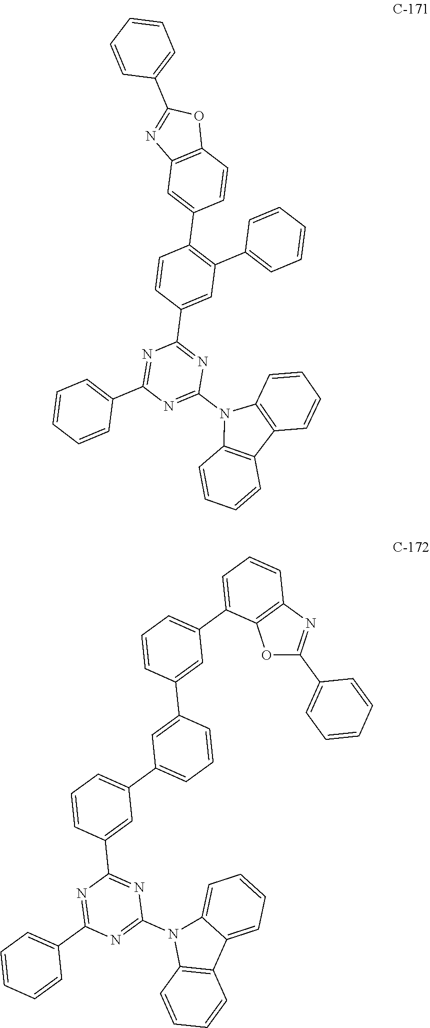 [SMILES: c1ccc(-c2nc(-c3ccc(-c4ccc5oc(-c6ccccc6)nc5c4)c(-c4ccccc4)c3)nc(-n3c4ccccc4c4ccccc43)n2)cc1 ; c1ccc(-c2nc(-c3cccc(-c4cccc(-c5cccc(-c6cccc7nc(-c8ccccc8)oc67)c5)c4)c3)nc(-n3c4ccccc4c4ccccc43)n2)cc1]

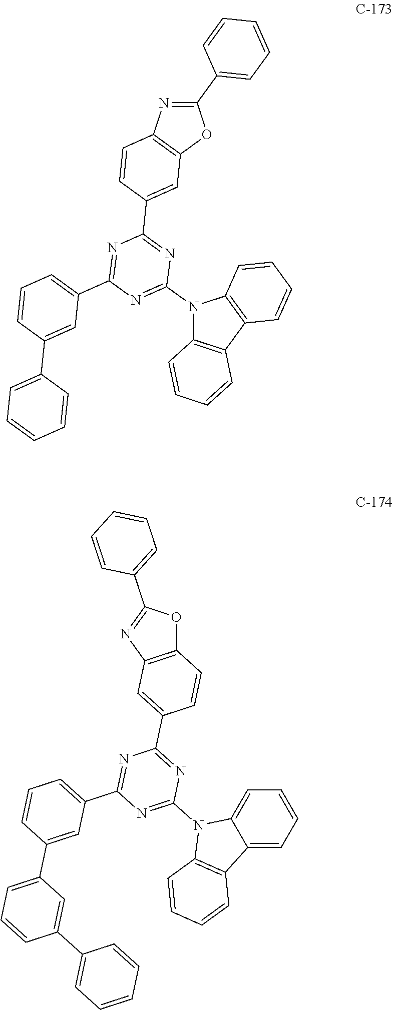 [SMILES: c1ccc(-c2cccc(-c3nc(-c4ccc5nc(-c6ccccc6)oc5c4)nc(-n4c5ccccc5c5ccccc54)n3)c2)cc1 ; c1ccc(-c2cccc(-c3cccc(-c4nc(-c5ccc6oc(-c7ccccc7)nc6c5)nc(-n5c6ccccc6c6ccccc65)n4)c3)c2)cc1]

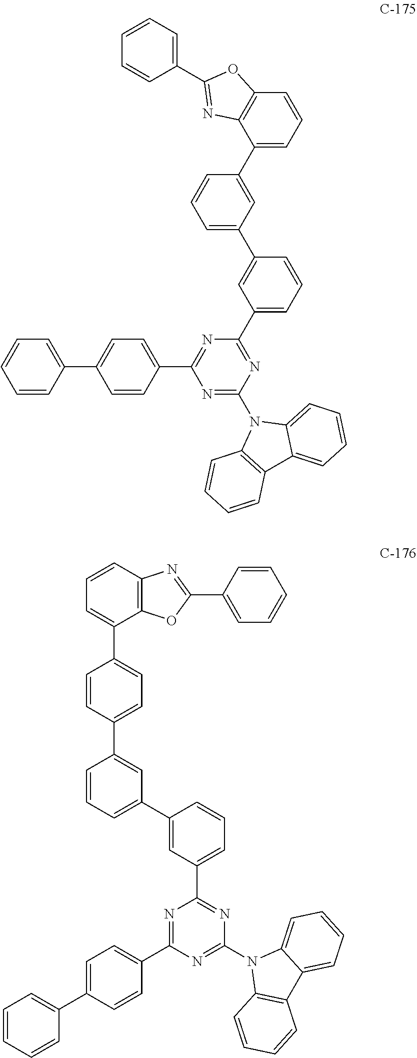 [SMILES: c1ccc(-c2ccc(-c3nc(-c4cccc(-c5cccc(-c6cccc7oc(-c8ccccc8)nc67)c5)c4)nc(-n4c5ccccc5c5ccccc54)n3)cc2)cc1 ; c1ccc(-c2ccc(-c3nc(-c4cccc(-c5cccc(-c6ccc(-c7cccc8nc(-c9ccccc9)oc78)cc6)c5)c4)nc(-n4c5ccccc5c5ccccc54)n3)cc2)cc1]

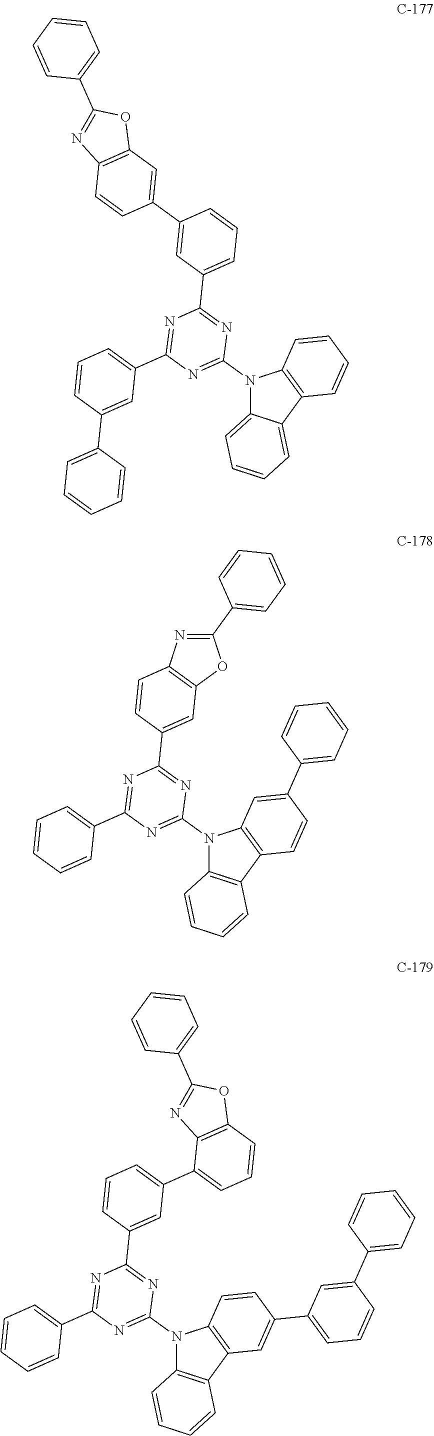 [SMILES: c1ccc(-c2cccc(-c3nc(-c4cccc(-c5ccc6nc(-c7ccccc7)oc6c5)c4)nc(-n4c5ccccc5c5ccccc54)n3)c2)cc1 ; c1ccc(-c2ccc3c4ccccc4n(-c4nc(-c5ccccc5)nc(-c5ccc6nc(-c7ccccc7)oc6c5)n4)c3c2)cc1 ; c1ccc(-c2cccc(-c3ccc4c(c3)c3ccccc3n4-c3nc(-c4ccccc4)nc(-c4cccc(-c5cccc6oc(-c7ccccc7)nc56)c4)n3)c2)cc1]

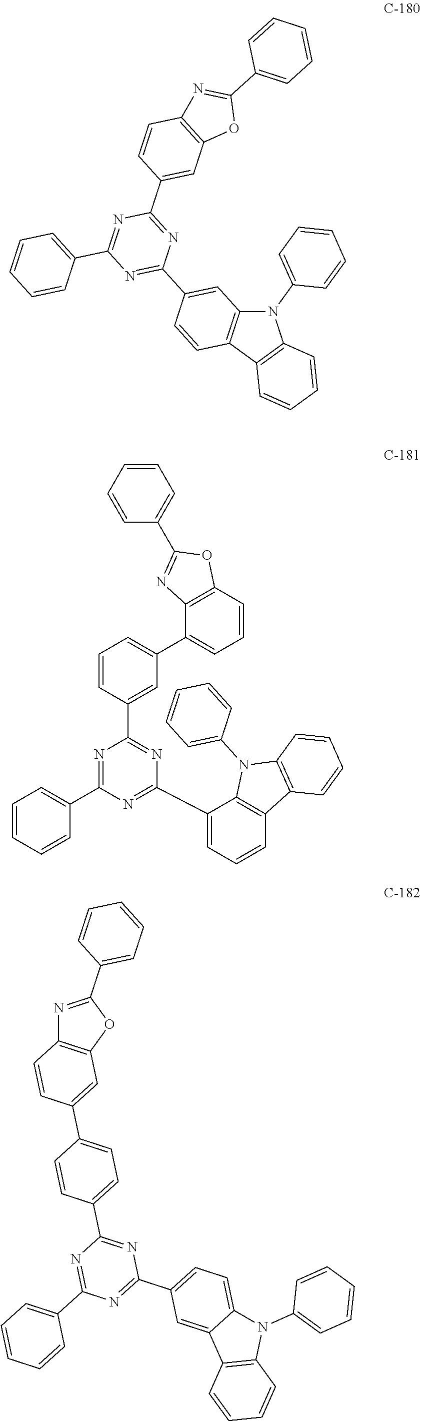 [SMILES: c1ccc(-c2nc(-c3ccc4nc(-c5ccccc5)oc4c3)nc(-c3ccc4c5ccccc5n(-c5ccccc5)c4c3)n2)cc1 ; c1ccc(-c2nc(-c3cccc(-c4cccc5oc(-c6ccccc6)nc45)c3)nc(-c3cccc4c5ccccc5n(-c5ccccc5)c34)n2)cc1 ; c1ccc(-c2nc(-c3ccc(-c4ccc5nc(-c6ccccc6)oc5c4)cc3)nc(-c3ccc4c(c3)c3ccccc3n4-c3ccccc3)n2)cc1]

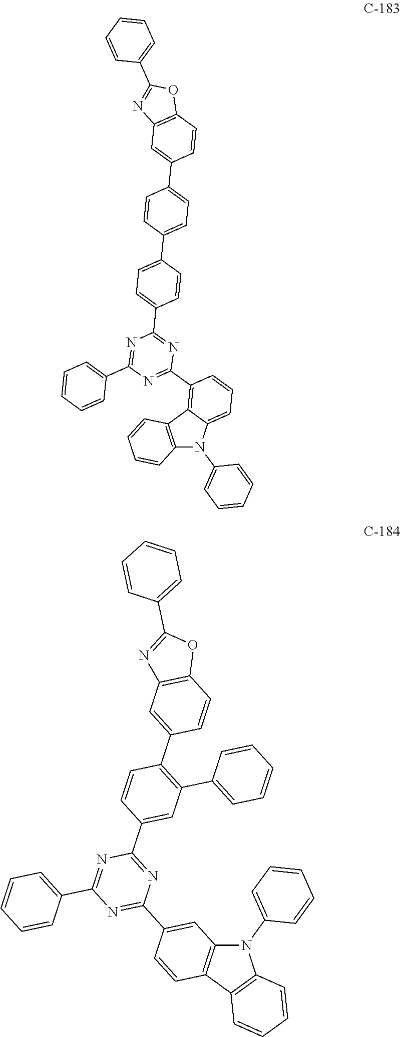 [SMILES: c1ccc(-c2nc(-c3ccc(-c4ccc(-c5ccc6oc(-c7ccccc7)nc6c5)cc4)cc3)nc(-c3cccc4c3c3ccccc3n4-c3ccccc3)n2)cc1 ; c1ccc(-c2nc(-c3ccc(-c4ccc5oc(-c6ccccc6)nc5c4)c(-c4ccccc4)c3)nc(-c3ccc4c5ccccc5n(-c5ccccc5)c4c3)n2)cc1]

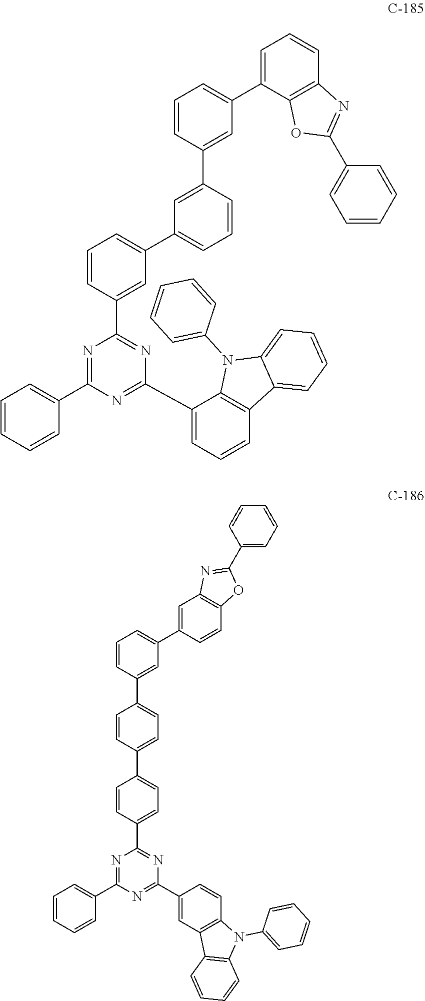 [SMILES: c1ccc(-c2nc(-c3cccc(-c4cccc(-c5cccc(-c6cccc7nc(-c8ccccc8)oc67)c5)c4)c3)nc(-c3cccc4c5ccccc5n(-c5ccccc5)c34)n2)cc1 ; c1ccc(-c2nc(-c3ccc(-c4ccc(-c5cccc(-c6ccc7oc(-c8ccccc8)nc7c6)c5)cc4)cc3)nc(-c3ccc4c(c3)c3ccccc3n4-c3ccccc3)n2)cc1]

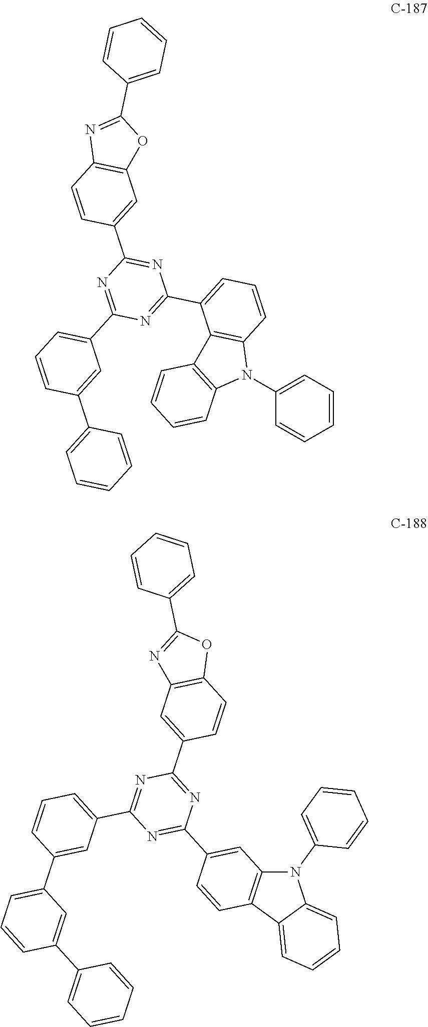 [SMILES: c1ccc(-c2cccc(-c3nc(-c4ccc5nc(-c6ccccc6)oc5c4)nc(-c4cccc5c4c4ccccc4n5-c4ccccc4)n3)c2)cc1 ; c1ccc(-c2cccc(-c3cccc(-c4nc(-c5ccc6oc(-c7ccccc7)nc6c5)nc(-c5ccc6c7ccccc7n(-c7ccccc7)c6c5)n4)c3)c2)cc1]

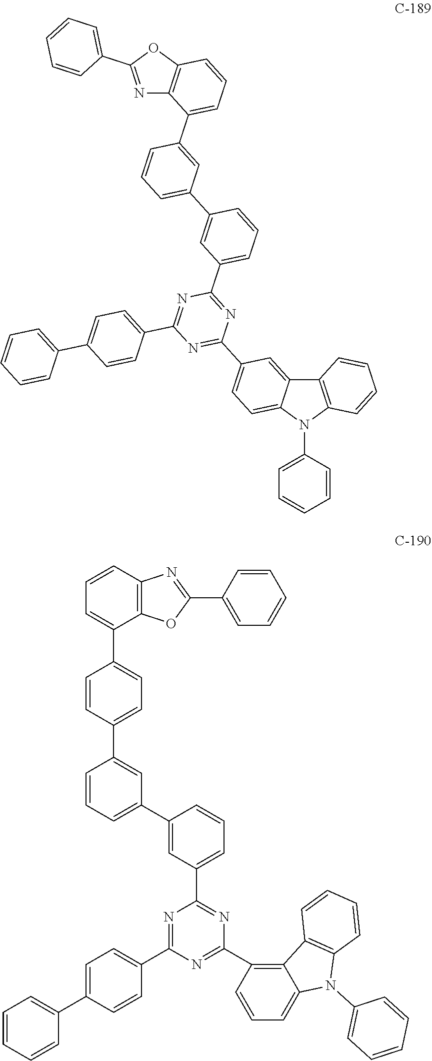 [SMILES: c1ccc(-c2ccc(-c3nc(-c4cccc(-c5cccc(-c6cccc7oc(-c8ccccc8)nc67)c5)c4)nc(-c4ccc5c(c4)c4ccccc4n5-c4ccccc4)n3)cc2)cc1 ; c1ccc(-c2ccc(-c3nc(-c4cccc(-c5cccc(-c6ccc(-c7cccc8nc(-c9ccccc9)oc78)cc6)c5)c4)nc(-c4cccc5c4c4ccccc4n5-c4ccccc4)n3)cc2)cc1]

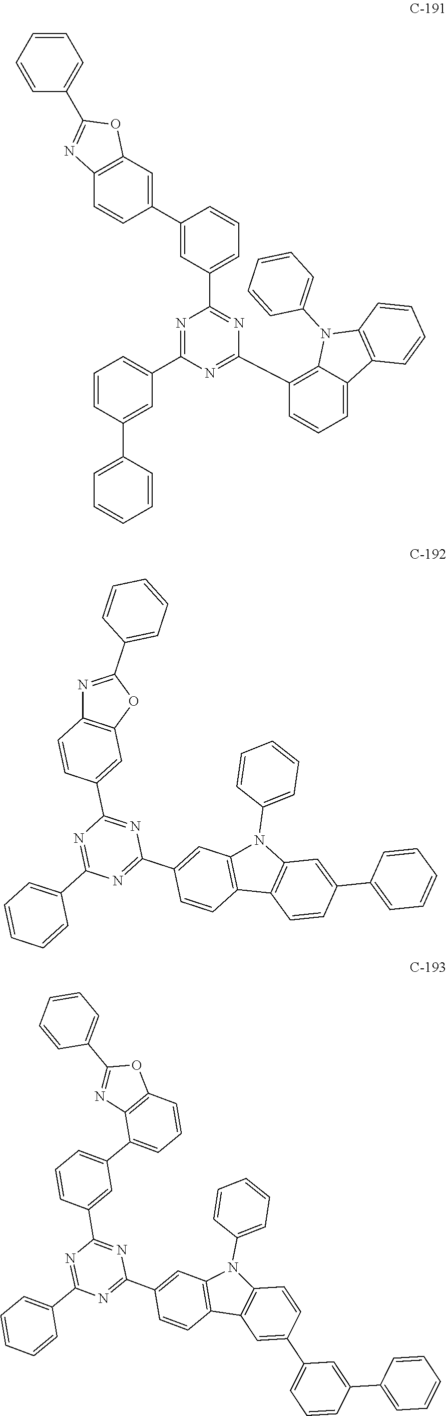 [SMILES: c1ccc(-c2cccc(-c3nc(-c4cccc(-c5ccc6nc(-c7ccccc7)oc6c5)c4)nc(-c4cccc5c6ccccc6n(-c6ccccc6)c45)n3)c2)cc1 ; c1ccc(-c2ccc3c4ccc(-c5nc(-c6ccccc6)nc(-c6ccc7nc(-c8ccccc8)oc7c6)n5)cc4n(-c4ccccc4)c3c2)cc1 ; c1ccc(-c2cccc(-c3ccc4c(c3)c3ccc(-c5nc(-c6ccccc6)nc(-c6cccc(-c7cccc8oc(-c9ccccc9)nc78)c6)n5)cc3n4-c3ccccc3)c2)cc1]

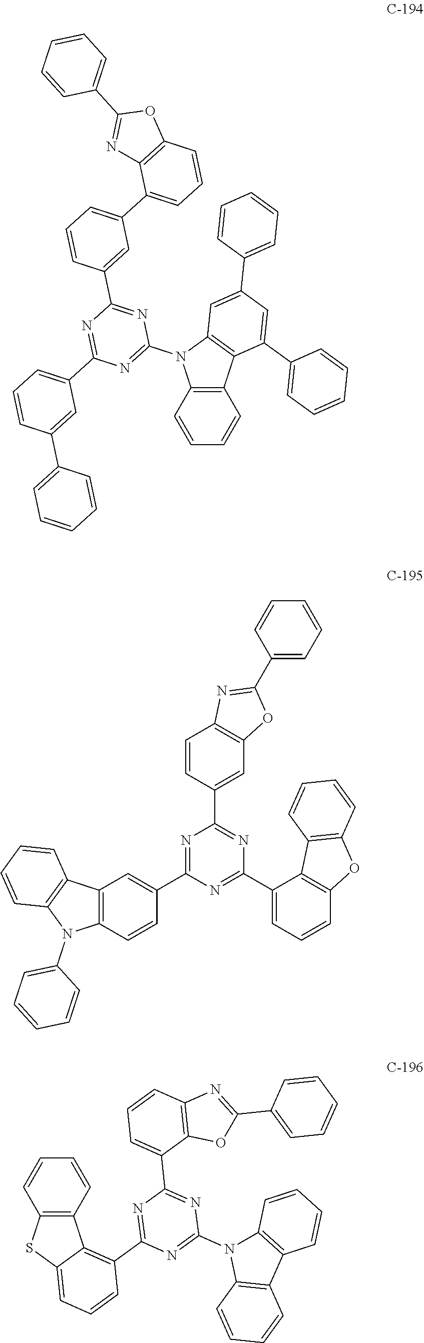 [SMILES: c1ccc(-c2cccc(-c3nc(-c4cccc(-c5cccc6oc(-c7ccccc7)nc56)c4)nc(-n4c5ccccc5c5c(-c6ccccc6)cc(-c6ccccc6)cc54)n3)c2)cc1 ; c1ccc(-c2nc3ccc(-c4nc(-c5ccc6c(c5)c5ccccc5n6-c5ccccc5)nc(-c5cccc6oc7ccccc7c56)n4)cc3o2)cc1 ; c1ccc(-c2nc3cccc(-c4nc(-c5cccc6sc7ccccc7c56)nc(-n5c6ccccc6c6ccccc65)n4)c3o2)cc1]

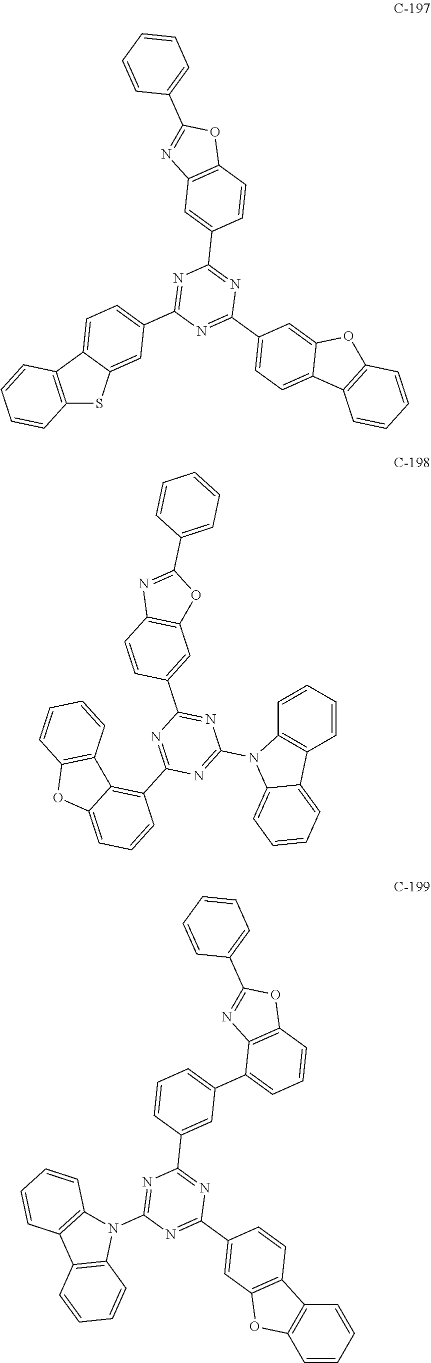 [SMILES: c1ccc(-c2nc3cc(-c4nc(-c5ccc6c(c5)oc5ccccc56)nc(-c5ccc6c(c5)sc5ccccc56)n4)ccc3o2)cc1 ; c1ccc(-c2nc3ccc(-c4nc(-c5cccc6oc7ccccc7c56)nc(-n5c6ccccc6c6ccccc65)n4)cc3o2)cc1 ; c1ccc(-c2nc3c(-c4cccc(-c5nc(-c6ccc7c(c6)oc6ccccc67)nc(-n6c7ccccc7c7ccccc76)n5)c4)cccc3o2)cc1]

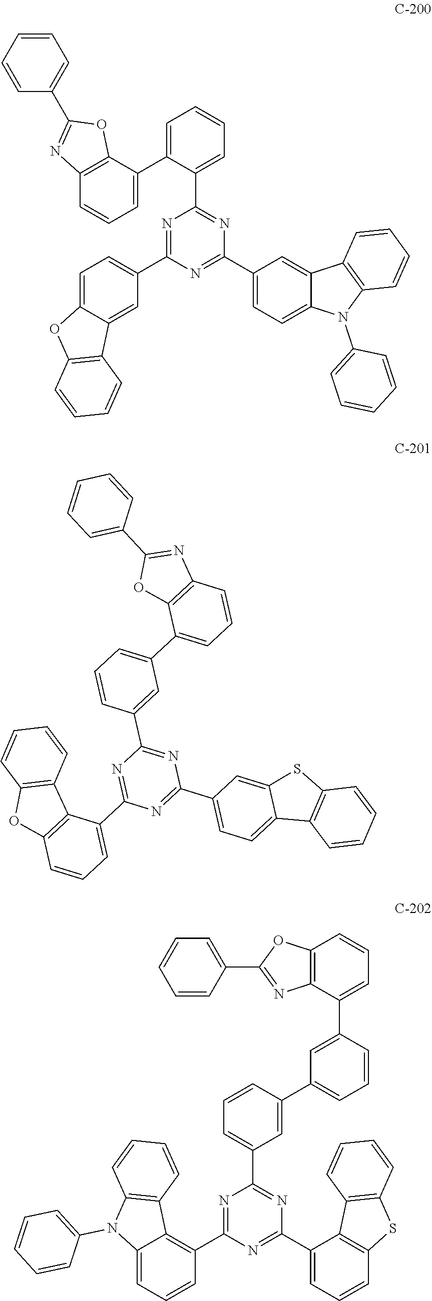 [SMILES: c1ccc(-c2nc3cccc(-c4ccccc4-c4nc(-c5ccc6oc7ccccc7c6c5)nc(-c5ccc6c(c5)c5ccccc5n6-c5ccccc5)n4)c3o2)cc1 ; c1ccc(-c2nc3cccc(-c4cccc(-c5nc(-c6ccc7c(c6)sc6ccccc67)nc(-c6cccc7oc8ccccc8c67)n5)c4)c3o2)cc1 ; c1ccc(-c2nc3c(-c4cccc(-c5cccc(-c6nc(-c7cccc8sc9ccccc9c78)nc(-c7cccc8c7c7ccccc7n8-c7ccccc7)n6)c5)c4)cccc3o2)cc1]

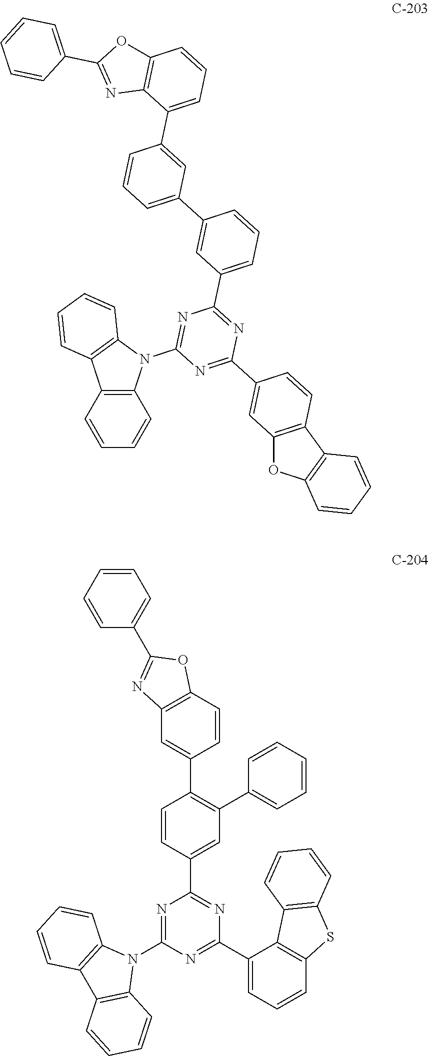 [SMILES: c1ccc(-c2nc3c(-c4cccc(-c5cccc(-c6nc(-c7ccc8c(c7)oc7ccccc78)nc(-n7c8ccccc8c8ccccc87)n6)c5)c4)cccc3o2)cc1 ; c1ccc(-c2nc3cc(-c4ccc(-c5nc(-c6cccc7sc8ccccc8c67)nc(-n6c7ccccc7c7ccccc76)n5)cc4-c4ccccc4)ccc3o2)cc1]

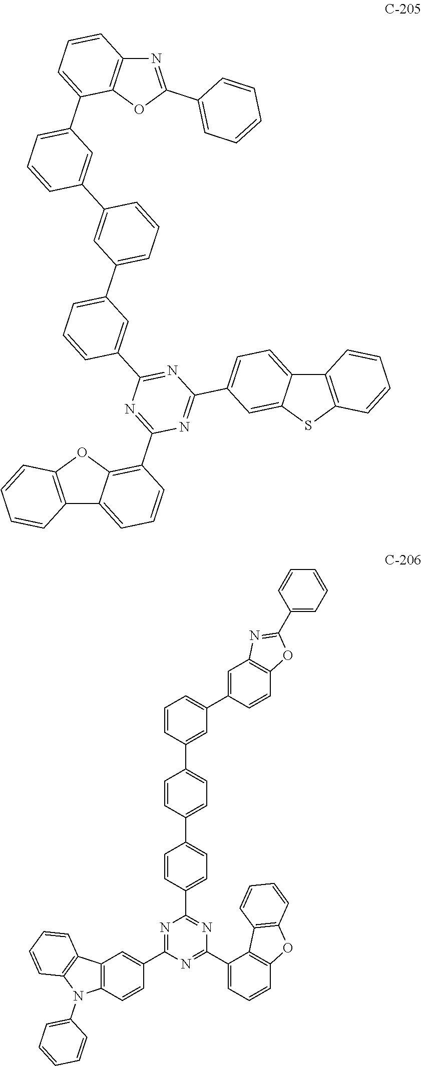 [SMILES: c1ccc(-c2nc3cccc(-c4cccc(-c5cccc(-c6cccc(-c7nc(-c8ccc9c(c8)sc8ccccc89)nc(-c8cccc9c8oc8ccccc89)n7)c6)c5)c4)c3o2)cc1 ; c1ccc(-c2nc3cc(-c4cccc(-c5ccc(-c6ccc(-c7nc(-c8ccc9c(c8)c8ccccc8n9-c8ccccc8)nc(-c8cccc9oc%10ccccc%10c89)n7)cc6)cc5)c4)ccc3o2)cc1]

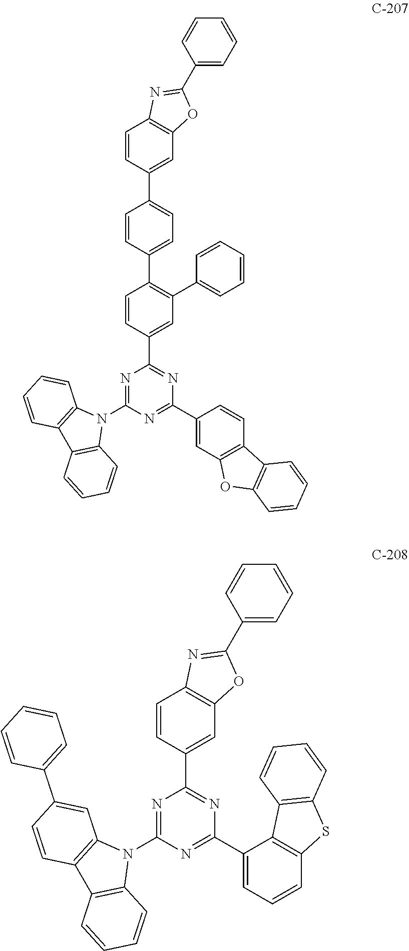 [SMILES: c1ccc(-c2nc3ccc(-c4ccc(-c5ccc(-c6nc(-c7ccc8c(c7)oc7ccccc78)nc(-n7c8ccccc8c8ccccc87)n6)cc5-c5ccccc5)cc4)cc3o2)cc1 ; c1ccc(-c2ccc3c4ccccc4n(-c4nc(-c5ccc6nc(-c7ccccc7)oc6c5)nc(-c5cccc6sc7ccccc7c56)n4)c3c2)cc1]

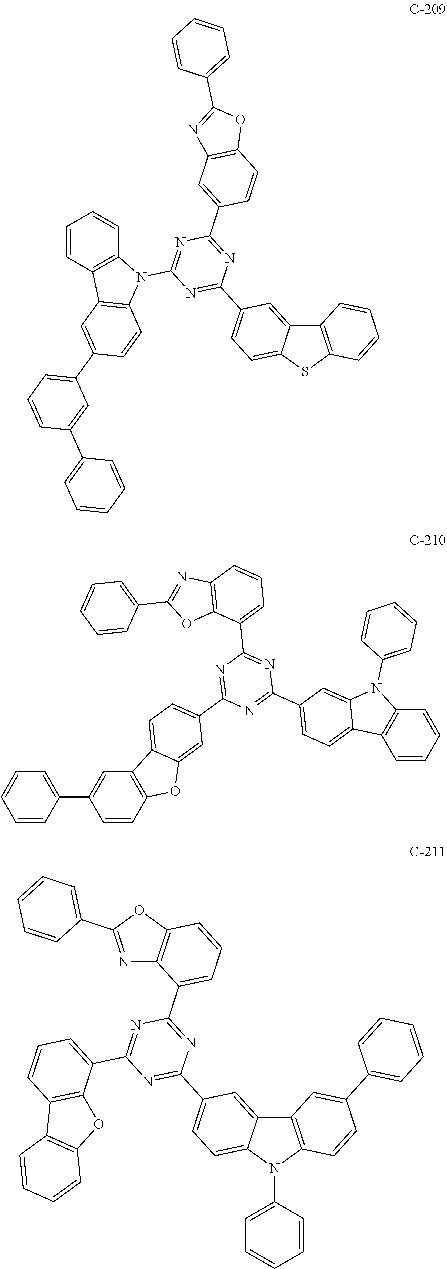 [SMILES: c1ccc(-c2cccc(-c3ccc4c(c3)c3ccccc3n4-c3nc(-c4ccc5oc(-c6ccccc6)nc5c4)nc(-c4ccc5sc6ccccc6c5c4)n3)c2)cc1 ; c1ccc(-c2ccc3oc4cc(-c5nc(-c6ccc7c8ccccc8n(-c8ccccc8)c7c6)nc(-c6cccc7nc(-c8ccccc8)oc67)n5)ccc4c3c2)cc1 ; c1ccc(-c2ccc3c(c2)c2cc(-c4nc(-c5cccc6oc(-c7ccccc7)nc56)nc(-c5cccc6c5oc5ccccc56)n4)ccc2n3-c2ccccc2)cc1]

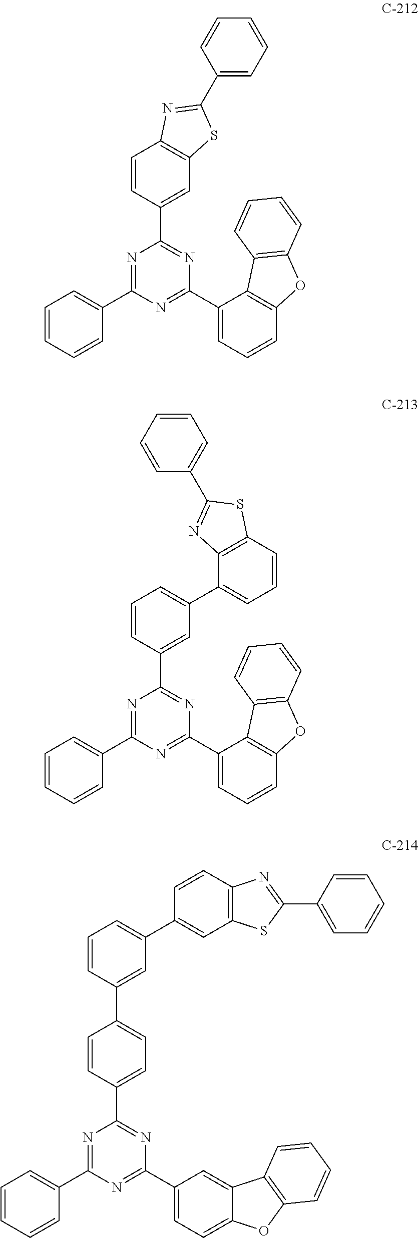 [SMILES: c1ccc(-c2nc(-c3ccc4nc(-c5ccccc5)sc4c3)nc(-c3cccc4oc5ccccc5c34)n2)cc1 ; c1ccc(-c2nc(-c3cccc(-c4cccc5sc(-c6ccccc6)nc45)c3)nc(-c3cccc4oc5ccccc5c34)n2)cc1 ; c1ccc(-c2nc(-c3ccc(-c4cccc(-c5ccc6nc(-c7ccccc7)sc6c5)c4)cc3)nc(-c3ccc4oc5ccccc5c4c3)n2)cc1]

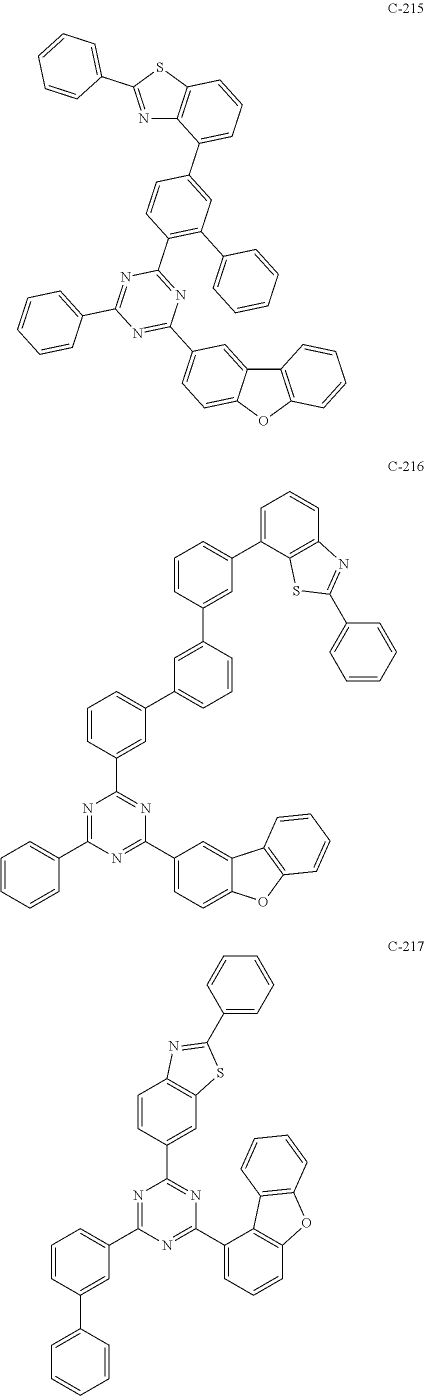 [SMILES: c1ccc(-c2nc(-c3ccc4oc5ccccc5c4c3)nc(-c3ccc(-c4cccc5sc(-c6ccccc6)nc45)cc3-c3ccccc3)n2)cc1 ; c1ccc(-c2nc(-c3cccc(-c4cccc(-c5cccc(-c6cccc7nc(-c8ccccc8)sc67)c5)c4)c3)nc(-c3ccc4oc5ccccc5c4c3)n2)cc1 ; c1ccc(-c2cccc(-c3nc(-c4ccc5nc(-c6ccccc6)sc5c4)nc(-c4cccc5oc6ccccc6c45)n3)c2)cc1]

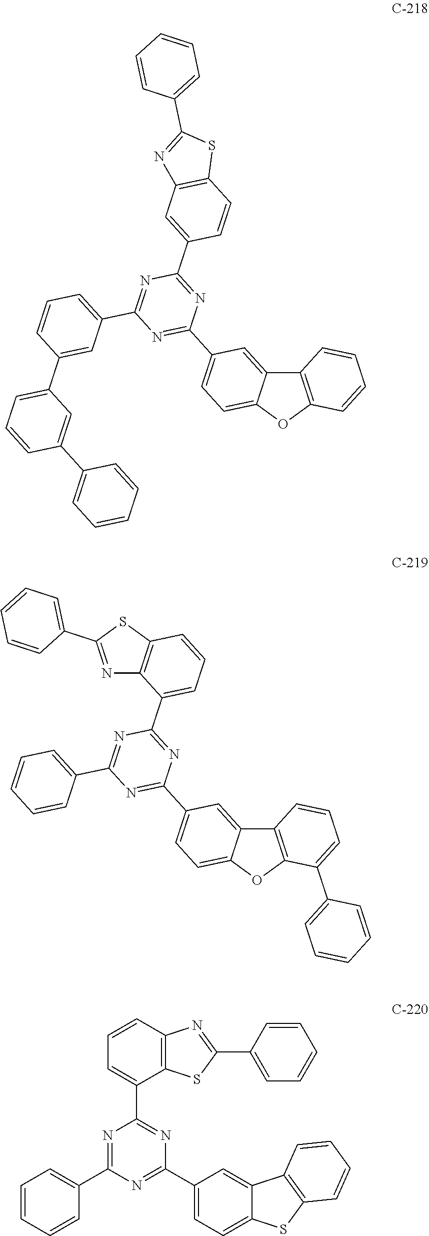 [SMILES: c1ccc(-c2cccc(-c3cccc(-c4nc(-c5ccc6sc(-c7ccccc7)nc6c5)nc(-c5ccc6oc7ccccc7c6c5)n4)c3)c2)cc1 ; c1ccc(-c2nc(-c3ccc4oc5c(-c6ccccc6)cccc5c4c3)nc(-c3cccc4sc(-c5ccccc5)nc34)n2)cc1 ; c1ccc(-c2nc(-c3ccc4sc5ccccc5c4c3)nc(-c3cccc4nc(-c5ccccc5)sc34)n2)cc1]

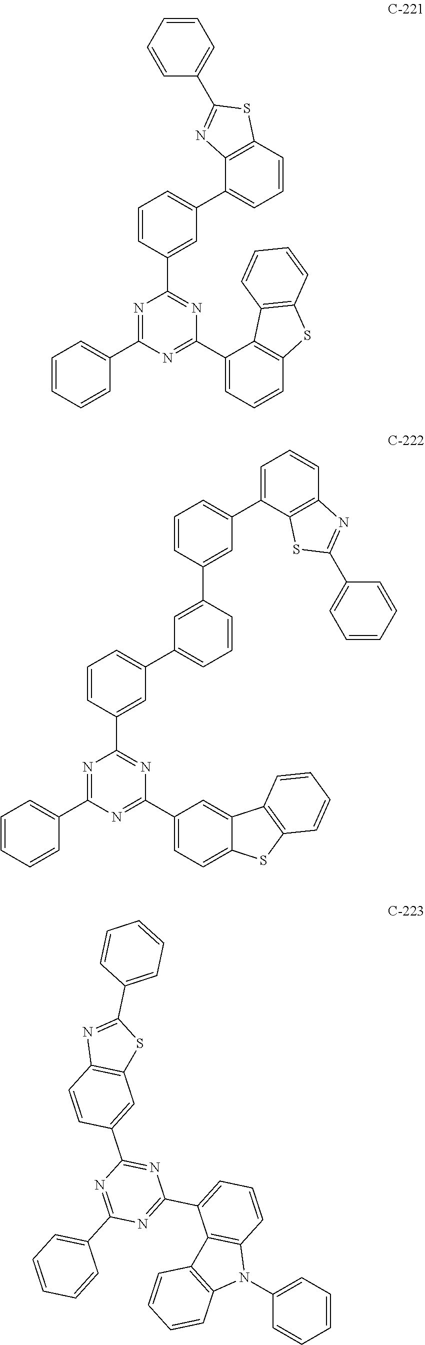 [SMILES: c1ccc(-c2nc(-c3cccc(-c4cccc5sc(-c6ccccc6)nc45)c3)nc(-c3cccc4sc5ccccc5c34)n2)cc1 ; c1ccc(-c2nc(-c3cccc(-c4cccc(-c5cccc(-c6cccc7nc(-c8ccccc8)sc67)c5)c4)c3)nc(-c3ccc4sc5ccccc5c4c3)n2)cc1 ; c1ccc(-c2nc(-c3ccc4nc(-c5ccccc5)sc4c3)nc(-c3cccc4c3c3ccccc3n4-c3ccccc3)n2)cc1]

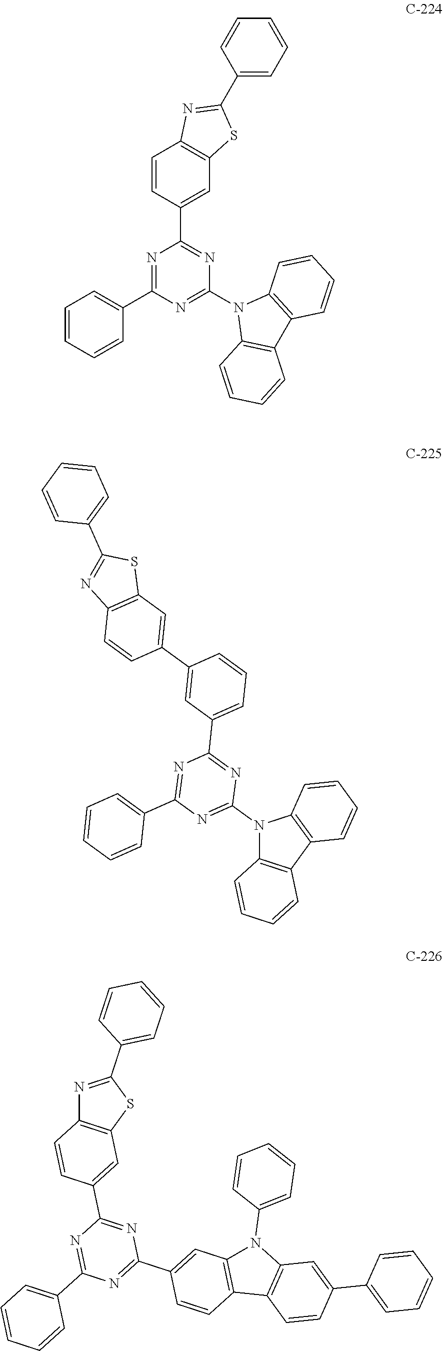 [SMILES: c1ccc(-c2nc(-c3ccc4nc(-c5ccccc5)sc4c3)nc(-n3c4ccccc4c4ccccc43)n2)cc1 ; c1ccc(-c2nc(-c3cccc(-c4ccc5nc(-c6ccccc6)sc5c4)c3)nc(-n3c4ccccc4c4ccccc43)n2)cc1 ; c1ccc(-c2ccc3c4ccc(-c5nc(-c6ccccc6)nc(-c6ccc7nc(-c8ccccc8)sc7c6)n5)cc4n(-c4ccccc4)c3c2)cc1]

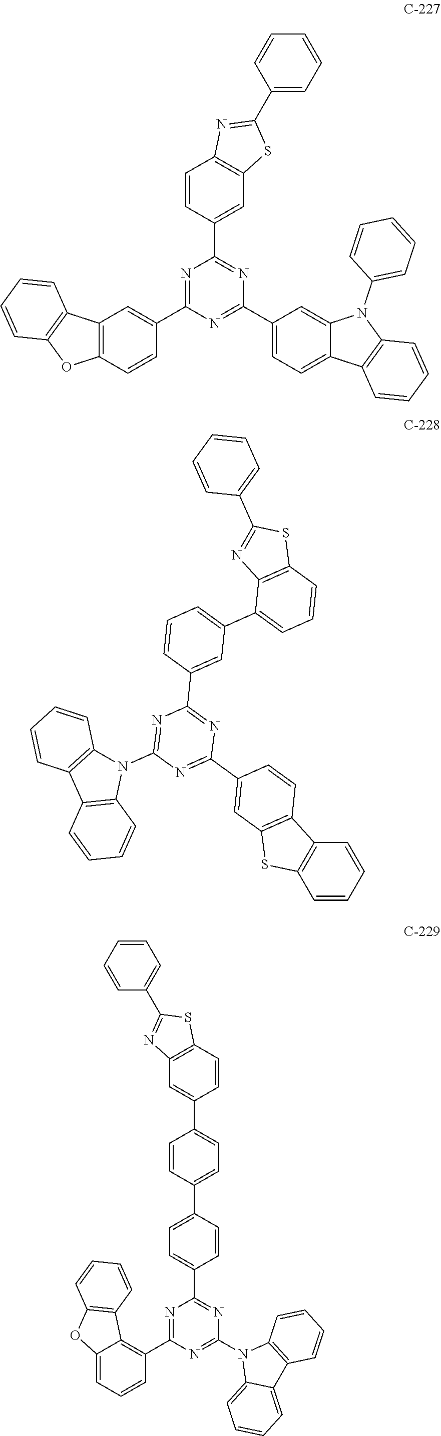 [SMILES: c1ccc(-c2nc3ccc(-c4nc(-c5ccc6oc7ccccc7c6c5)nc(-c5ccc6c7ccccc7n(-c7ccccc7)c6c5)n4)cc3s2)cc1 ; c1ccc(-c2nc3c(-c4cccc(-c5nc(-c6ccc7c(c6)sc6ccccc67)nc(-n6c7ccccc7c7ccccc76)n5)c4)cccc3s2)cc1 ; c1ccc(-c2nc3cc(-c4ccc(-c5ccc(-c6nc(-c7cccc8oc9ccccc9c78)nc(-n7c8ccccc8c8ccccc87)n6)cc5)cc4)ccc3s2)cc1]

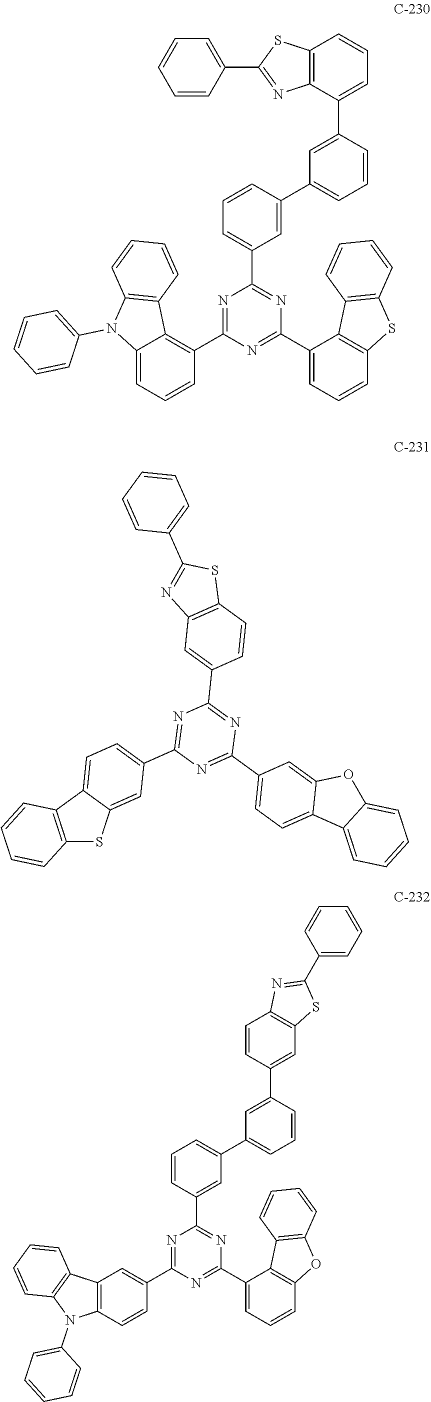 [SMILES: c1ccc(-c2nc3c(-c4cccc(-c5cccc(-c6nc(-c7cccc8sc9ccccc9c78)nc(-c7cccc8c7c7ccccc7n8-c7ccccc7)n6)c5)c4)cccc3s2)cc1 ; c1ccc(-c2nc3cc(-c4nc(-c5ccc6c(c5)oc5ccccc56)nc(-c5ccc6c(c5)sc5ccccc56)n4)ccc3s2)cc1 ; c1ccc(-c2nc3ccc(-c4cccc(-c5cccc(-c6nc(-c7ccc8c(c7)c7ccccc7n8-c7ccccc7)nc(-c7cccc8oc9ccccc9c78)n6)c5)c4)cc3s2)cc1]

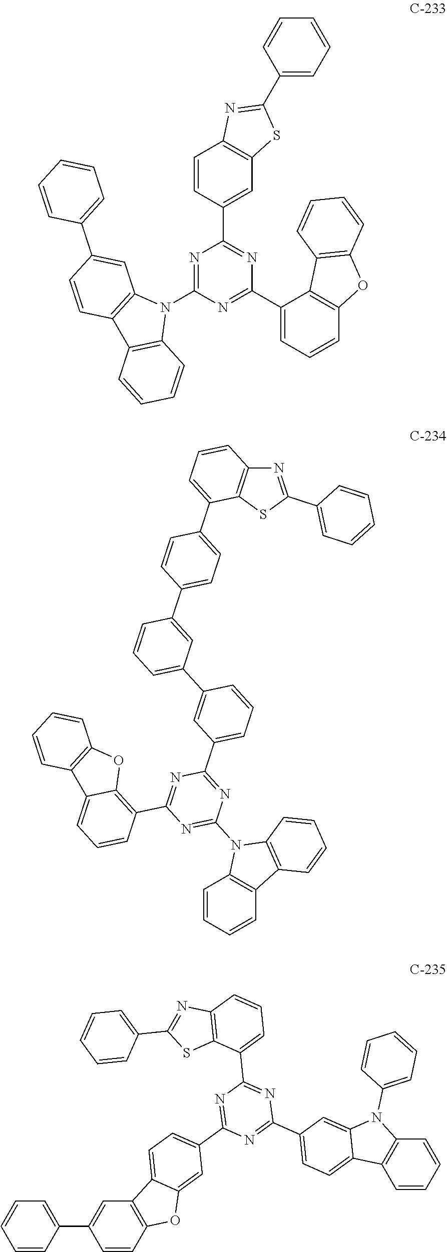 [SMILES: c1ccc(-c2ccc3c4ccccc4n(-c4nc(-c5ccc6nc(-c7ccccc7)sc6c5)nc(-c5cccc6oc7ccccc7c56)n4)c3c2)cc1 ; c1ccc(-c2nc3cccc(-c4ccc(-c5cccc(-c6cccc(-c7nc(-c8cccc9c8oc8ccccc89)nc(-n8c9ccccc9c9ccccc98)n7)c6)c5)cc4)c3s2)cc1 ; c1ccc(-c2ccc3oc4cc(-c5nc(-c6ccc7c8ccccc8n(-c8ccccc8)c7c6)nc(-c6cccc7nc(-c8ccccc8)sc67)n5)ccc4c3c2)cc1]

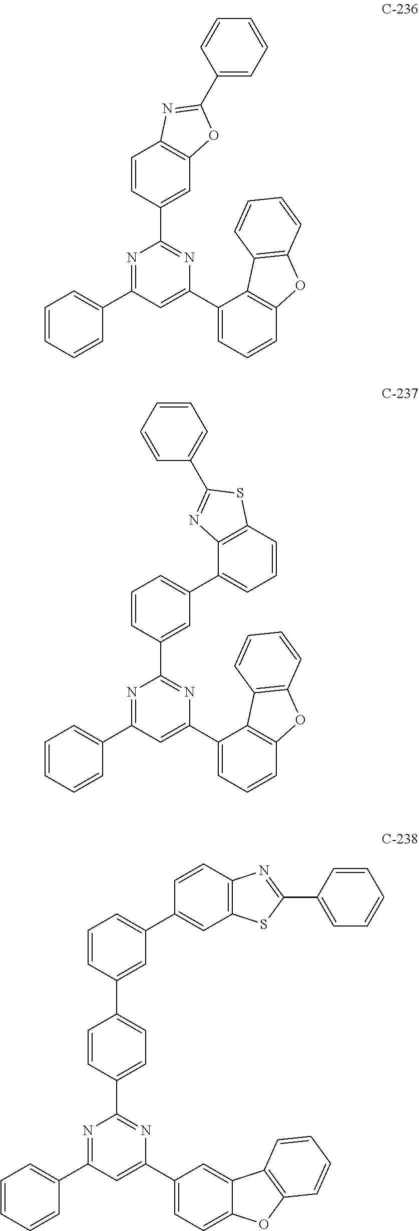 [SMILES: c1ccc(-c2cc(-c3cccc4oc5ccccc5c34)nc(-c3ccc4nc(-c5ccccc5)oc4c3)n2)cc1 ; c1ccc(-c2cc(-c3cccc4oc5ccccc5c34)nc(-c3cccc(-c4cccc5sc(-c6ccccc6)nc45)c3)n2)cc1 ; c1ccc(-c2cc(-c3ccc4oc5ccccc5c4c3)nc(-c3ccc(-c4cccc(-c5ccc6nc(-c7ccccc7)sc6c5)c4)cc3)n2)cc1]

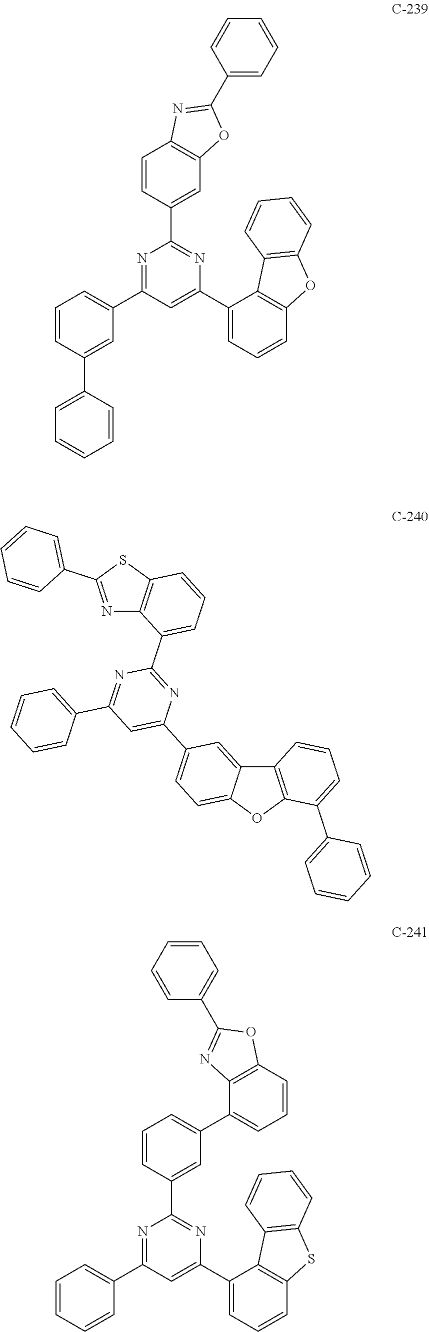 [SMILES: c1ccc(-c2cccc(-c3cc(-c4cccc5oc6ccccc6c45)nc(-c4ccc5nc(-c6ccccc6)oc5c4)n3)c2)cc1 ; c1ccc(-c2cc(-c3ccc4oc5c(-c6ccccc6)cccc5c4c3)nc(-c3cccc4sc(-c5ccccc5)nc34)n2)cc1 ; c1ccc(-c2cc(-c3cccc4sc5ccccc5c34)nc(-c3cccc(-c4cccc5oc(-c6ccccc6)nc45)c3)n2)cc1]

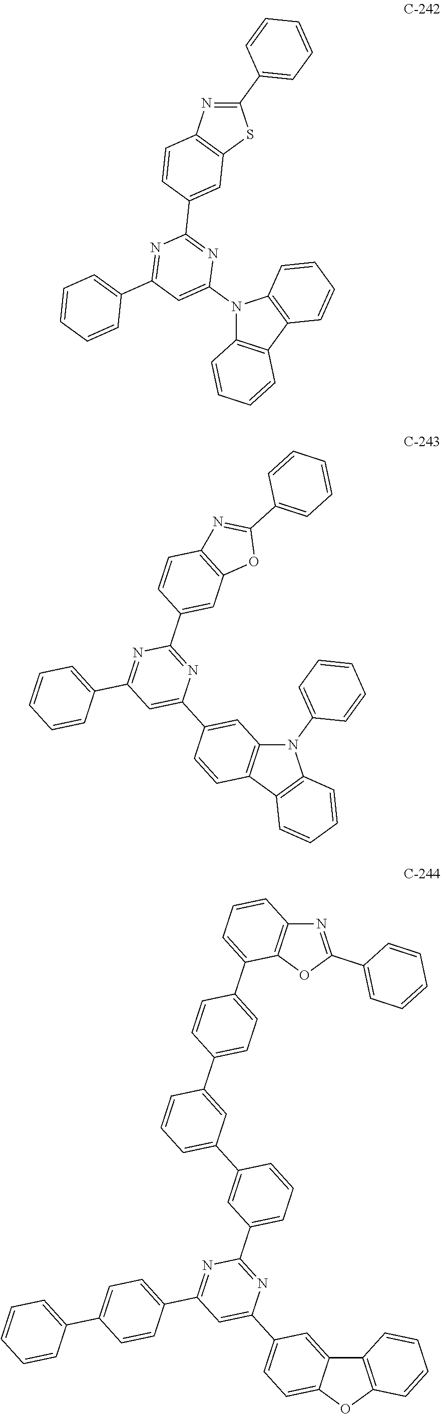 [SMILES: c1ccc(-c2cc(-n3c4ccccc4c4ccccc43)nc(-c3ccc4nc(-c5ccccc5)sc4c3)n2)cc1 ; c1ccc(-c2cc(-c3ccc4c5ccccc5n(-c5ccccc5)c4c3)nc(-c3ccc4nc(-c5ccccc5)oc4c3)n2)cc1 ; c1ccc(-c2ccc(-c3cc(-c4ccc5oc6ccccc6c5c4)nc(-c4cccc(-c5cccc(-c6ccc(-c7cccc8nc(-c9ccccc9)oc78)cc6)c5)c4)n3)cc2)cc1]

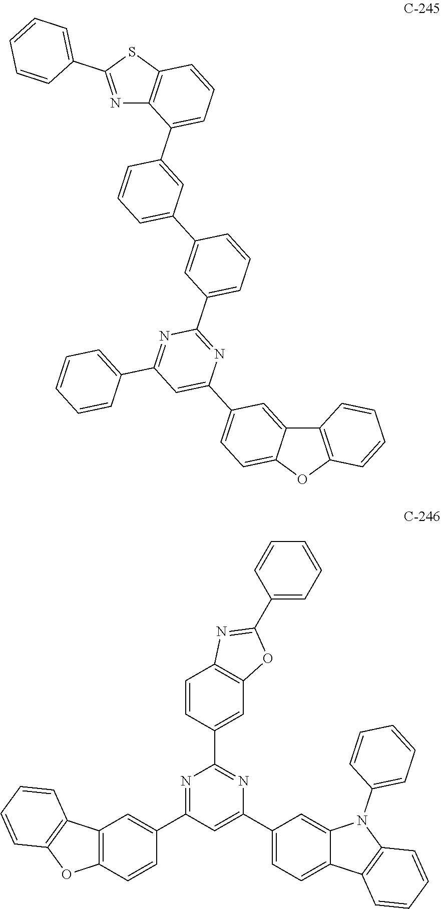 [SMILES: c1ccc(-c2cc(-c3ccc4oc5ccccc5c4c3)nc(-c3cccc(-c4cccc(-c5cccc6sc(-c7ccccc7)nc56)c4)c3)n2)cc1 ; c1ccc(-c2nc3ccc(-c4nc(-c5ccc6oc7ccccc7c6c5)cc(-c5ccc6c7ccccc7n(-c7ccccc7)c6c5)n4)cc3o2)cc1]

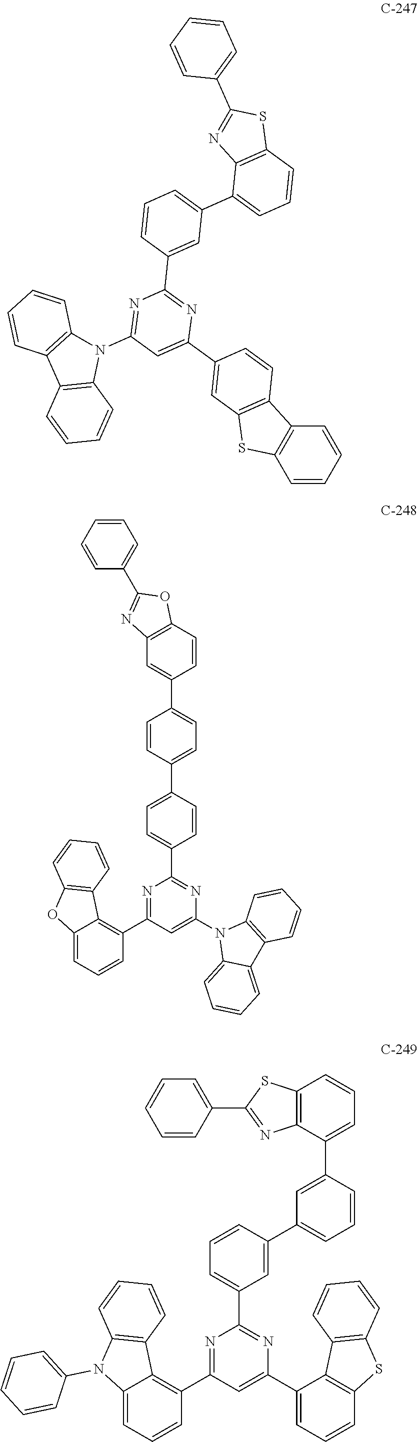 [SMILES: c1ccc(-c2nc3c(-c4cccc(-c5nc(-c6ccc7c(c6)sc6ccccc67)cc(-n6c7ccccc7c7ccccc76)n5)c4)cccc3s2)cc1 ; c1ccc(-c2nc3cc(-c4ccc(-c5ccc(-c6nc(-c7cccc8oc9ccccc9c78)cc(-n7c8ccccc8c8ccccc87)n6)cc5)cc4)ccc3o2)cc1 ; c1ccc(-c2nc3c(-c4cccc(-c5cccc(-c6nc(-c7cccc8sc9ccccc9c78)cc(-c7cccc8c7c7ccccc7n8-c7ccccc7)n6)c5)c4)cccc3s2)cc1]

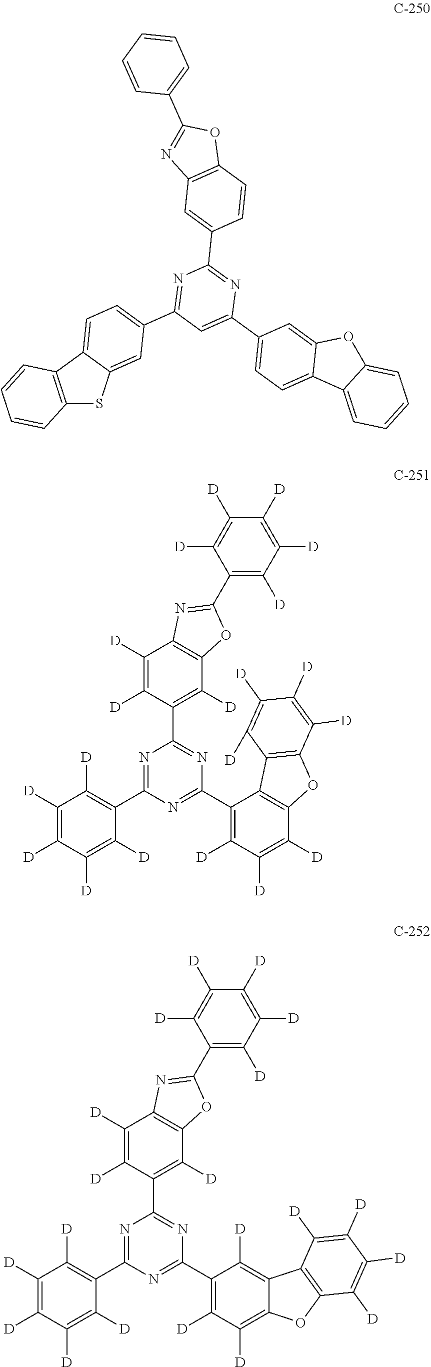 [SMILES: c1ccc(-c2nc3cc(-c4nc(-c5ccc6c(c5)oc5ccccc56)cc(-c5ccc6c(c5)sc5ccccc56)n4)ccc3o2)cc1 ; [2H]c1c([2H])c([2H])c(-c2nc(-c3c([2H])c([2H])c4nc(-c5c([2H])c([2H])c([2H])c([2H])c5[2H])oc4c3[2H])nc(-c3c([2H])c([2H])c([2H])c4oc5c([2H])c([2H])c([2H])c([2H])c5c34)n2)c([2H])c1[2H] ; [2H]c1c([2H])c([2H])c(-c2nc(-c3c([2H])c([2H])c4nc(-c5c([2H])c([2H])c([2H])c([2H])c5[2H])oc4c3[2H])nc(-c3c([2H])c([2H])c4oc5c([2H])c([2H])c([2H])c([2H])c5c4c3[2H])n2)c([2H])c1[2H]]

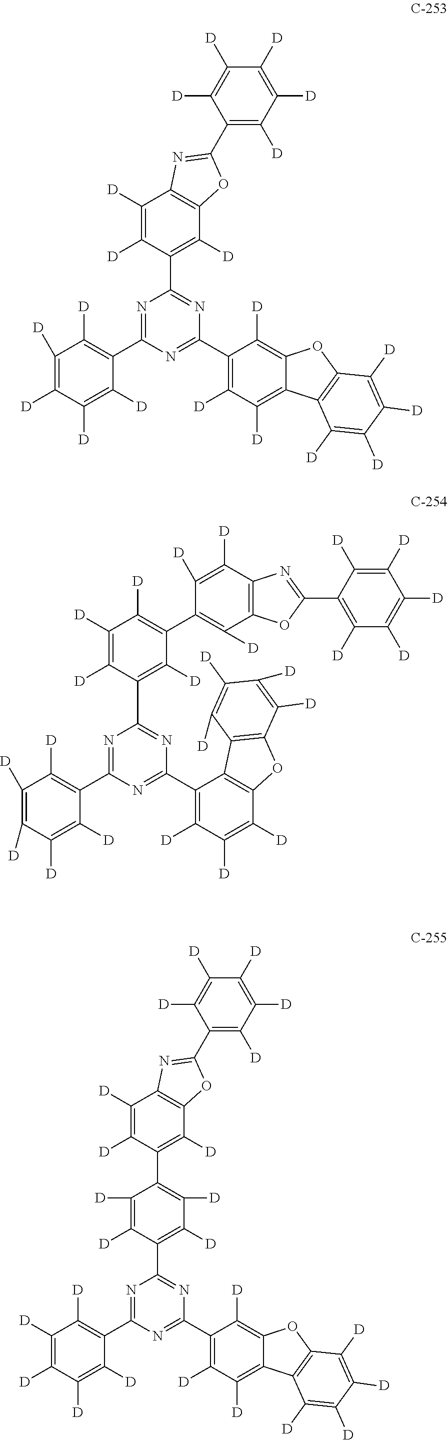

The compound represented by formula 2 may be at least one selected from the following compounds, but is not limited thereto.

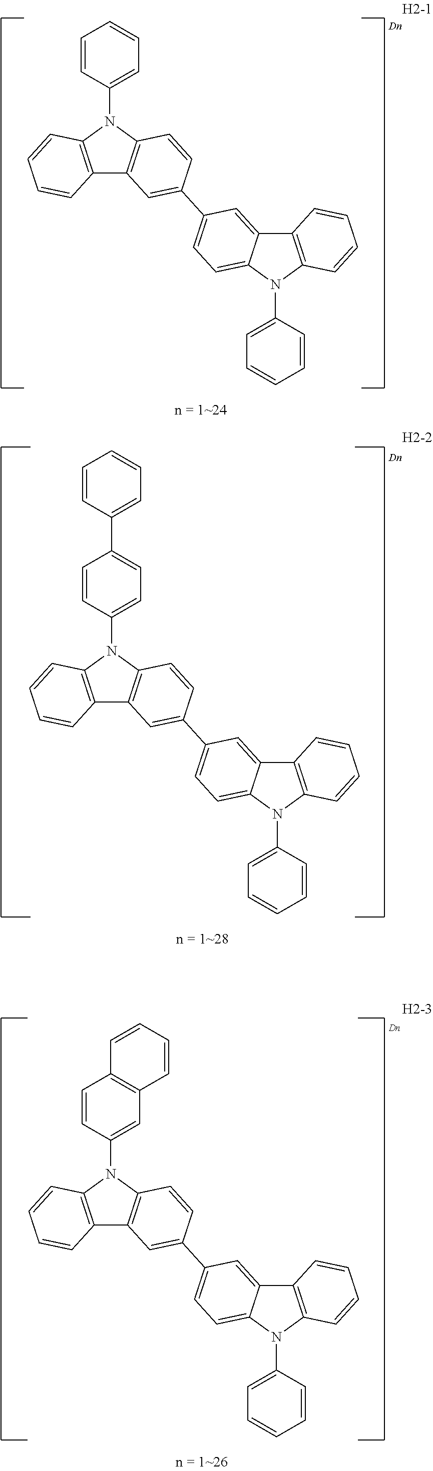

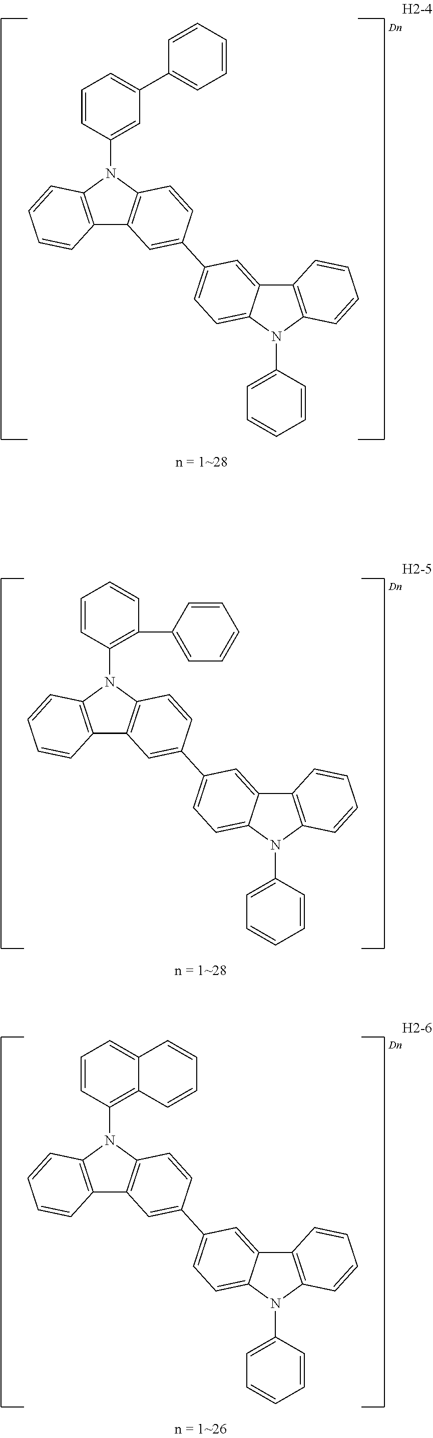

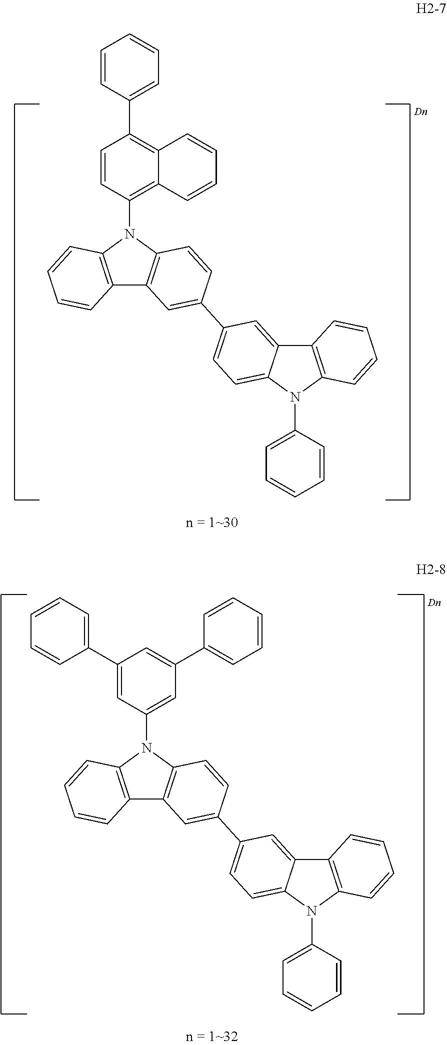

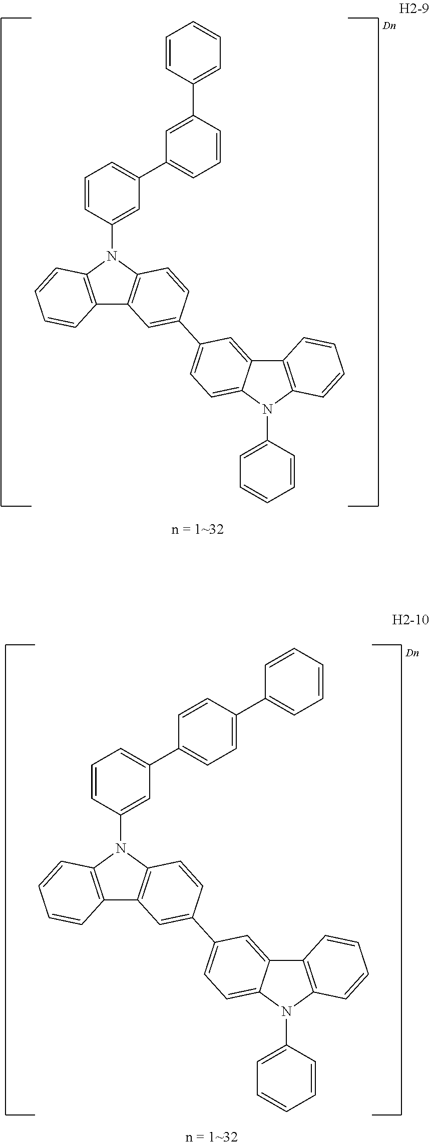

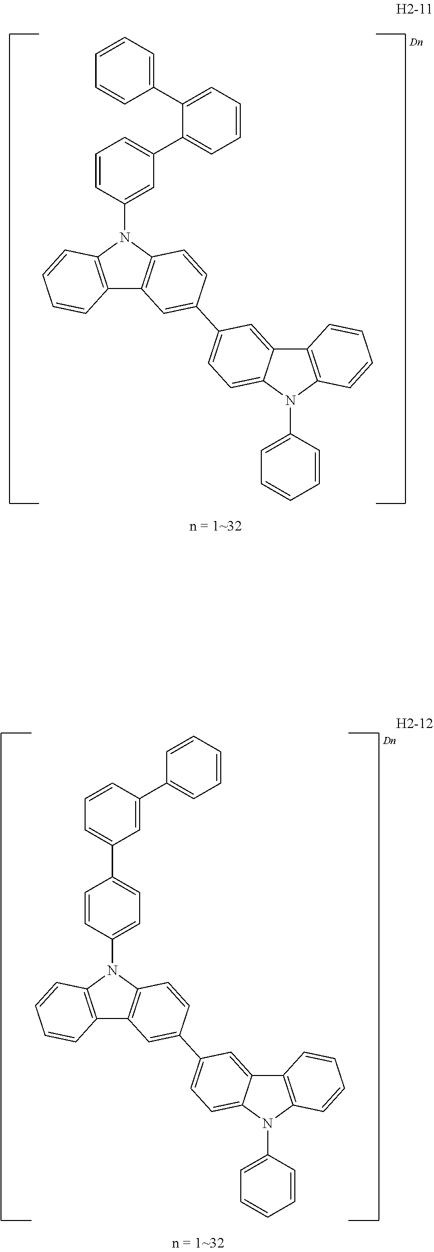

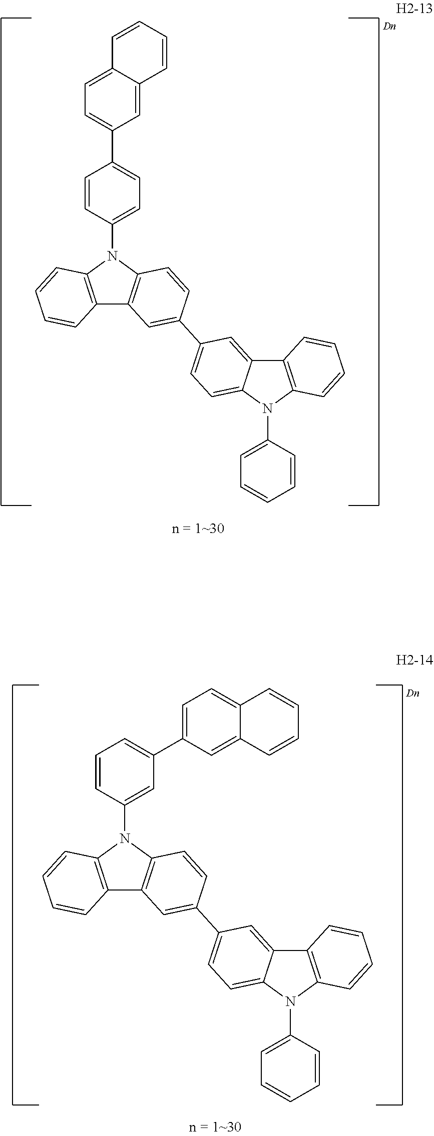

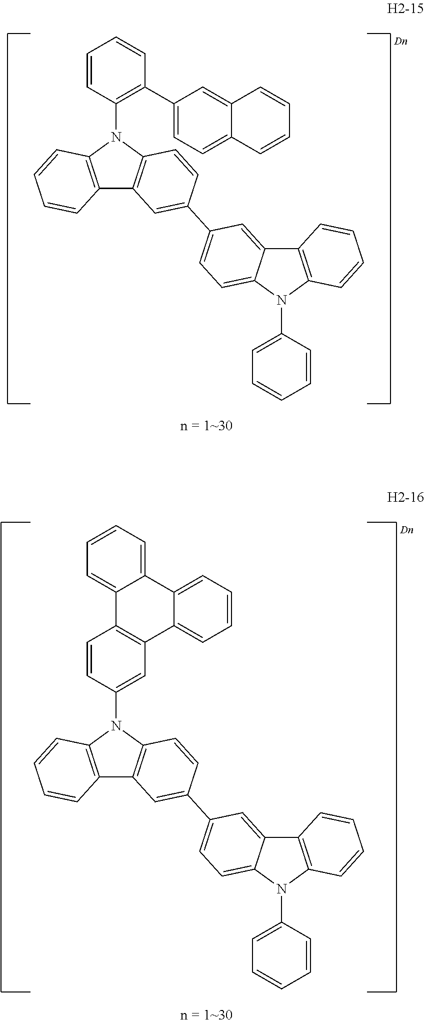

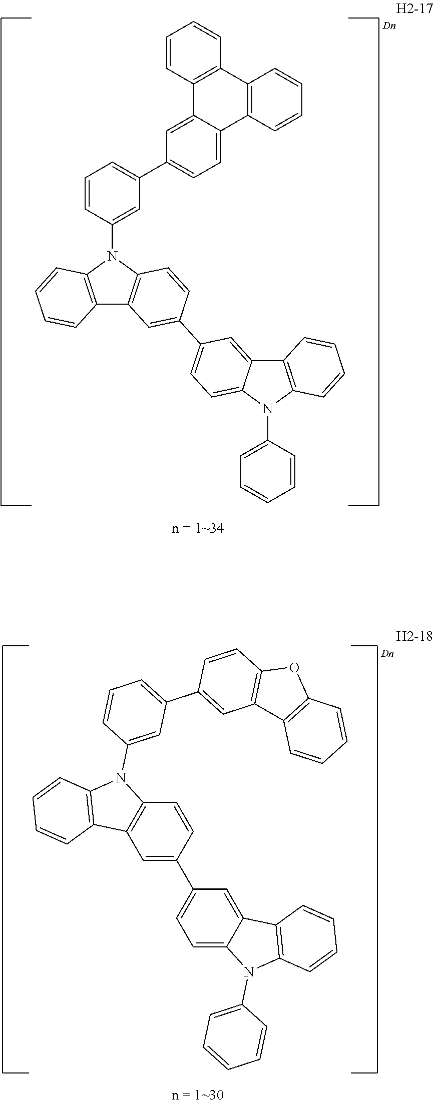

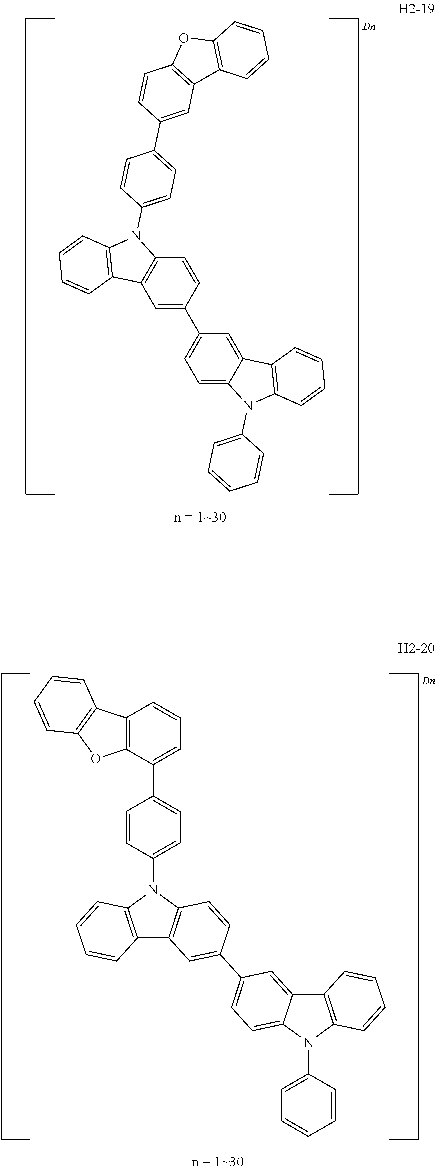

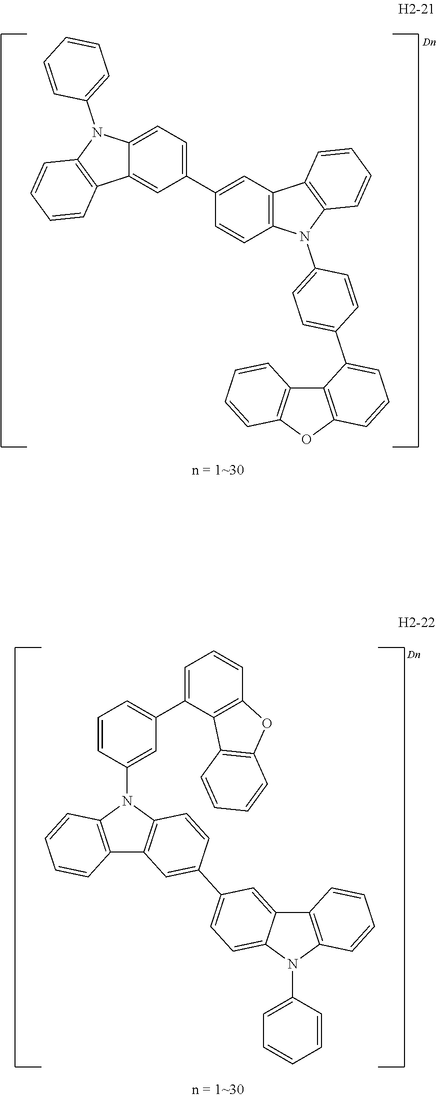

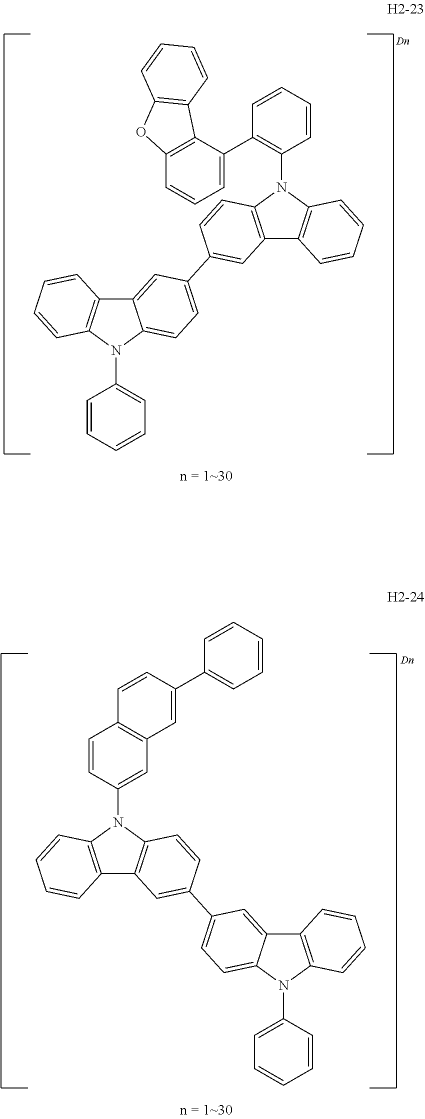

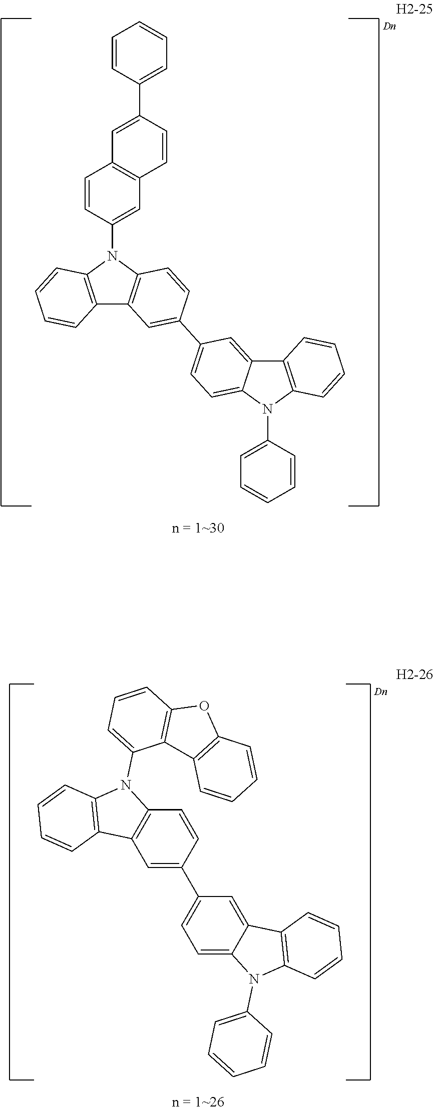

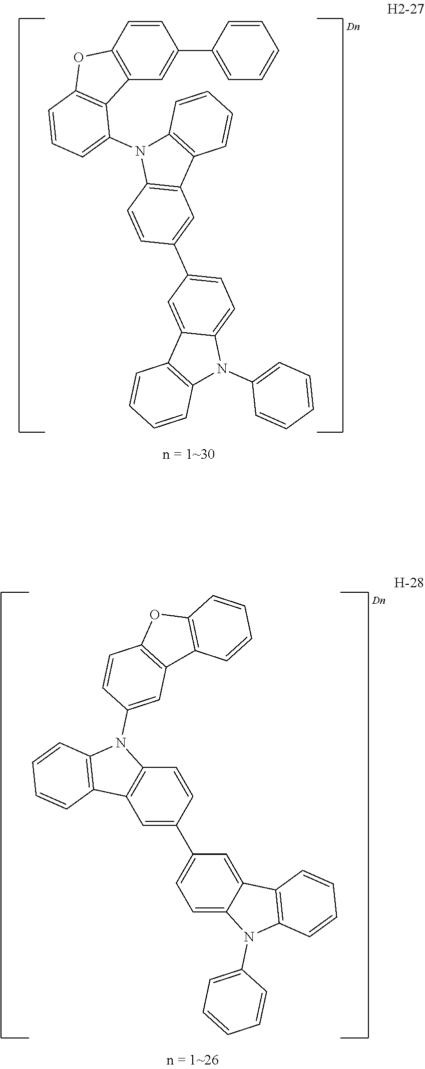

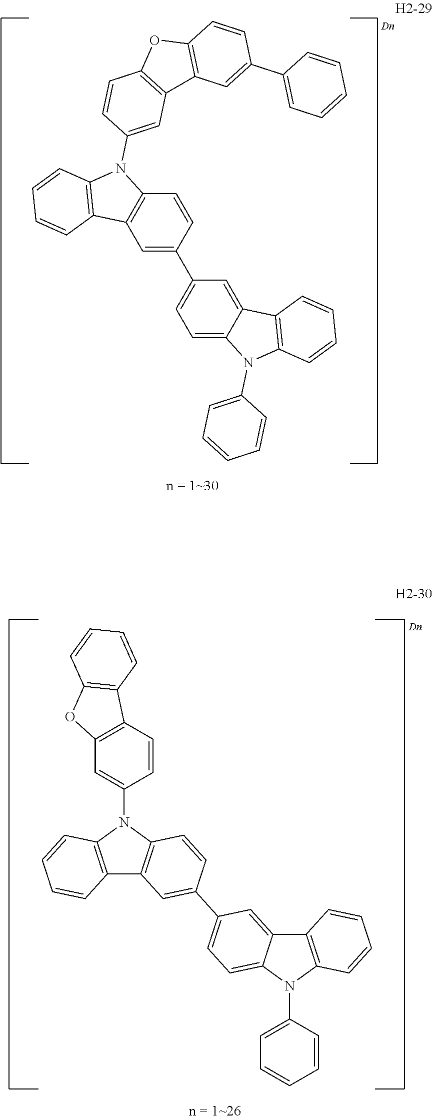

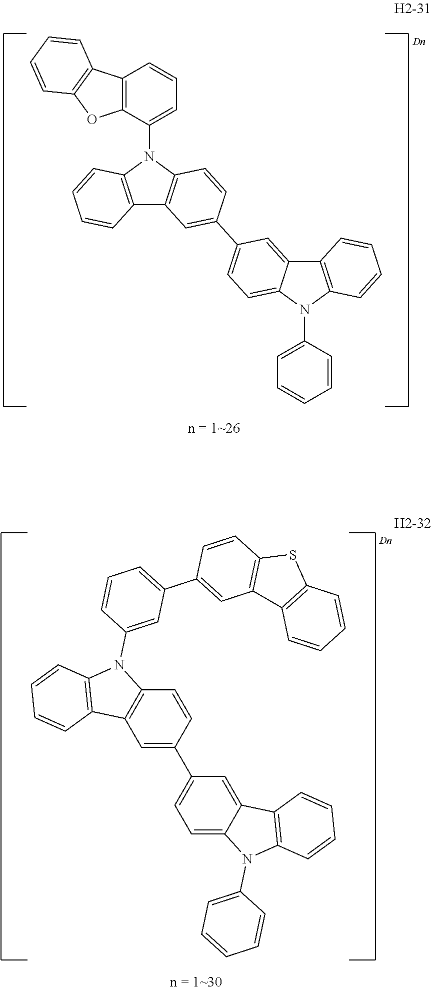

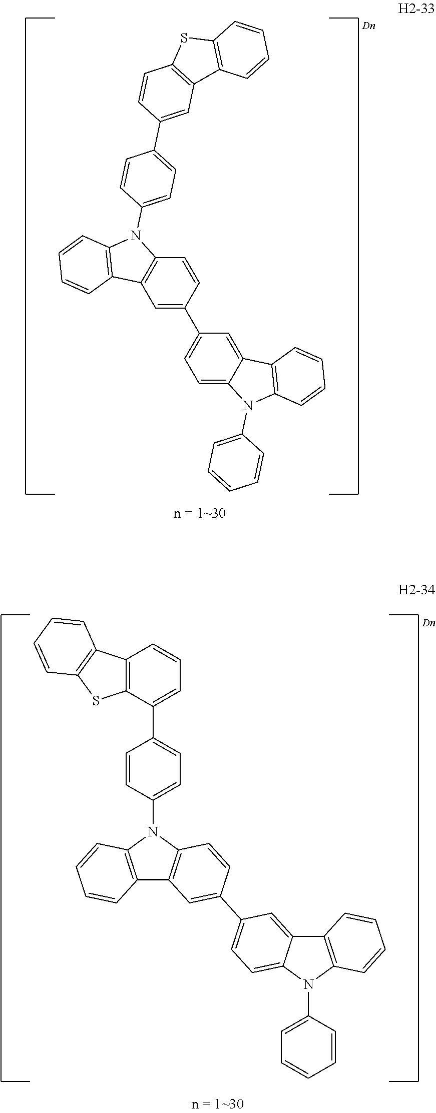

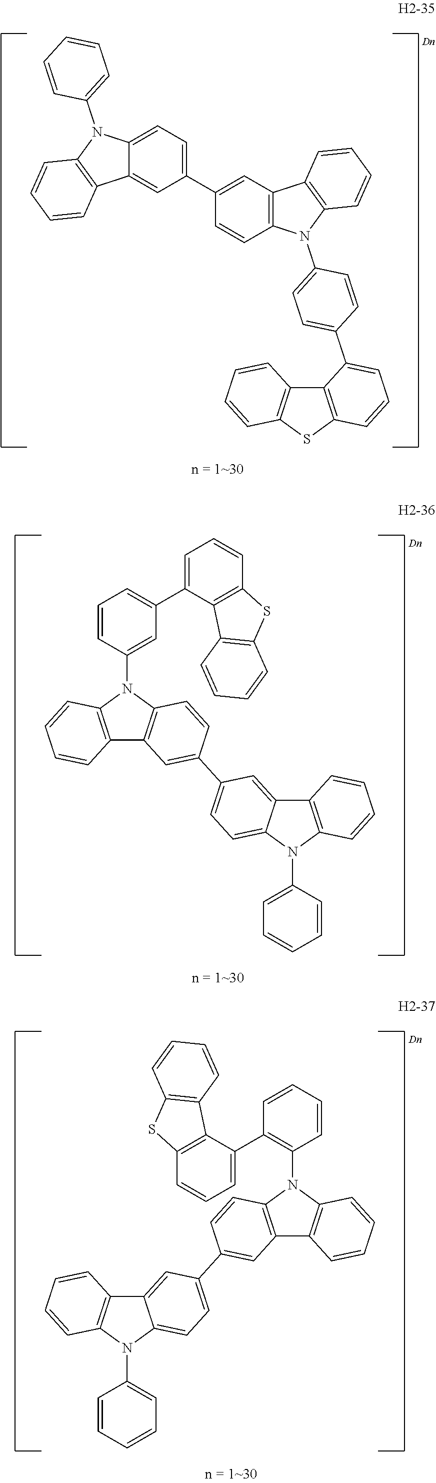

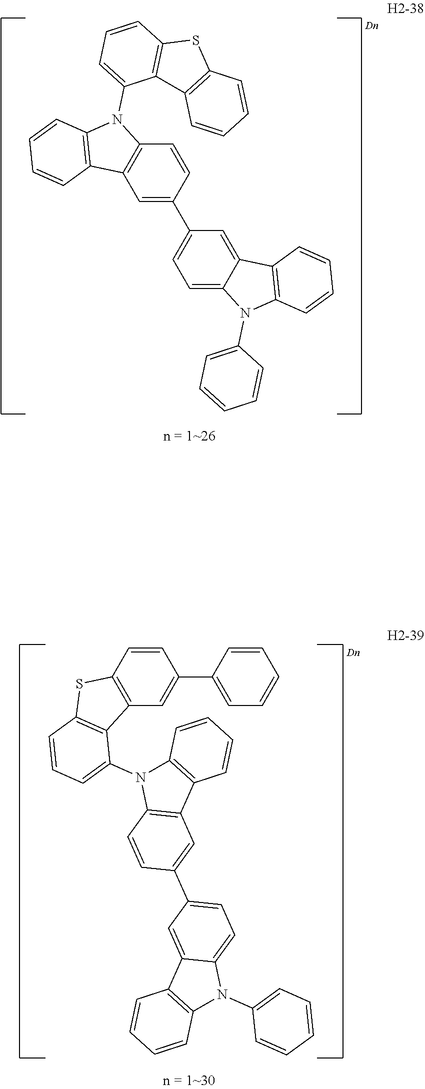

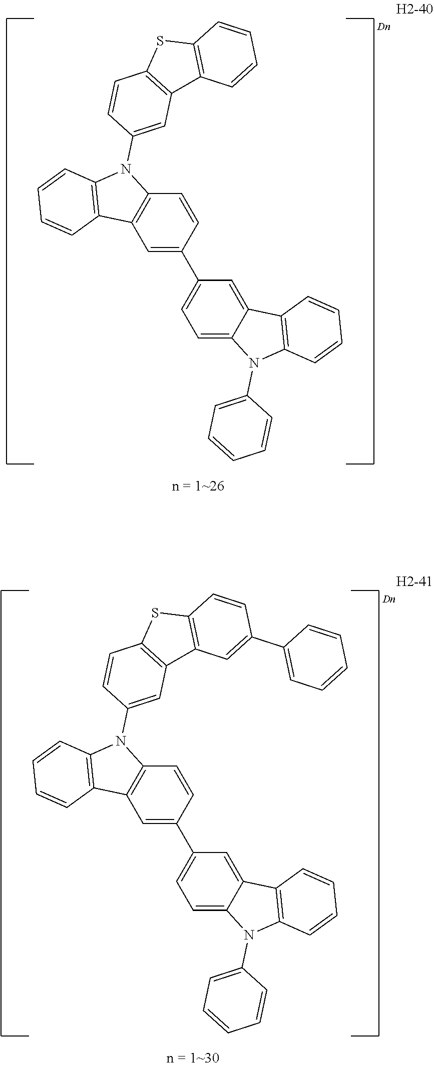

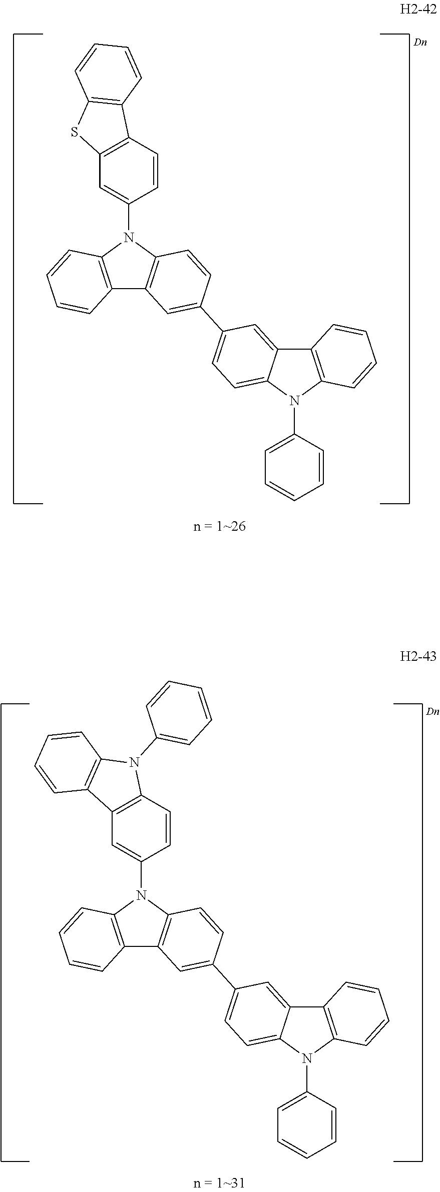

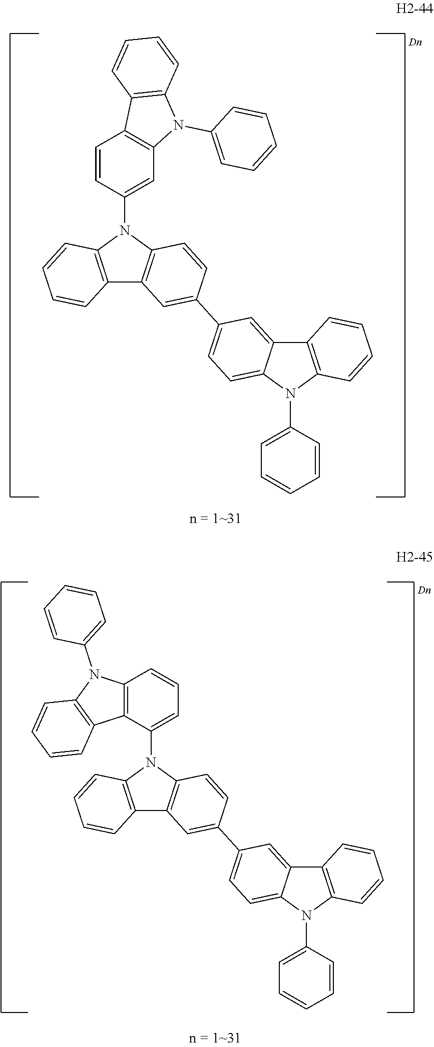

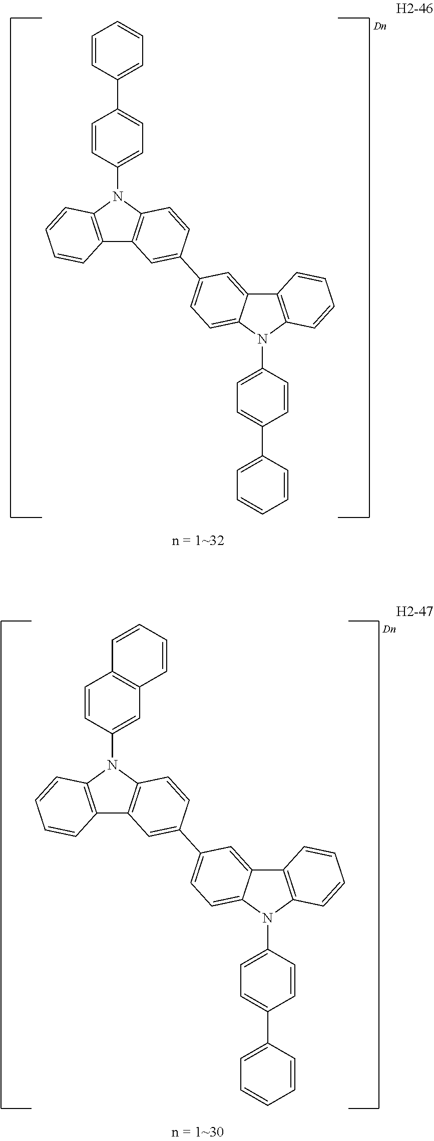

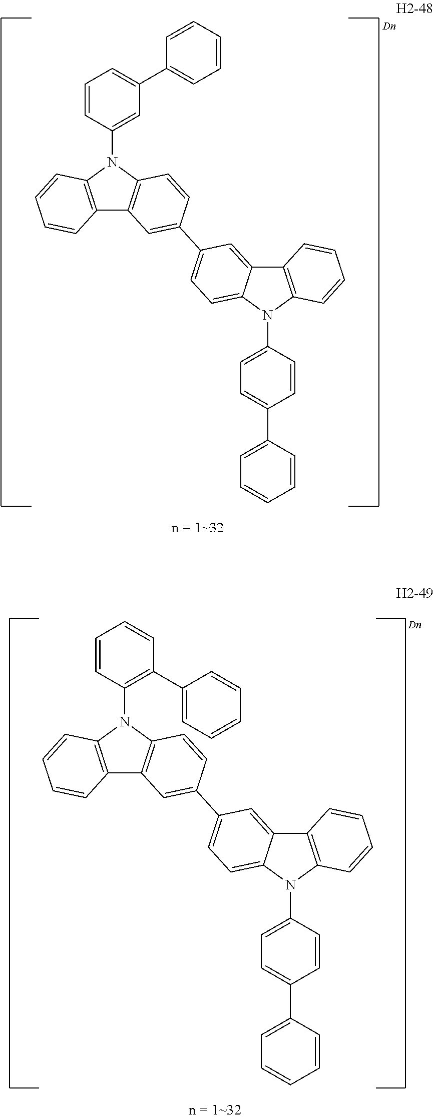

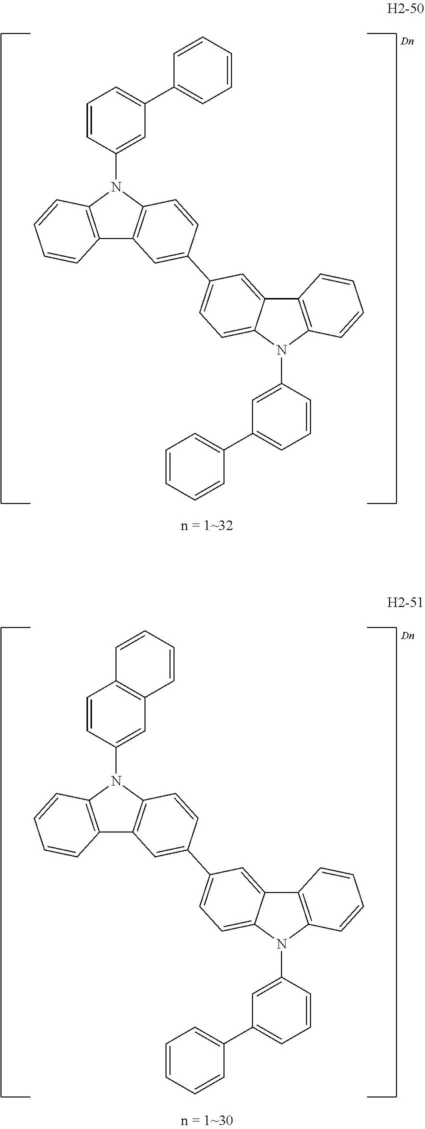

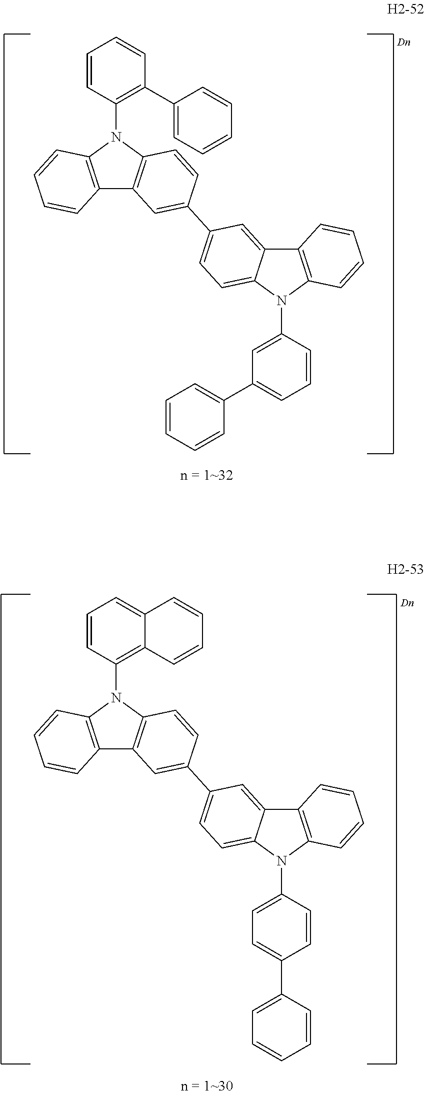

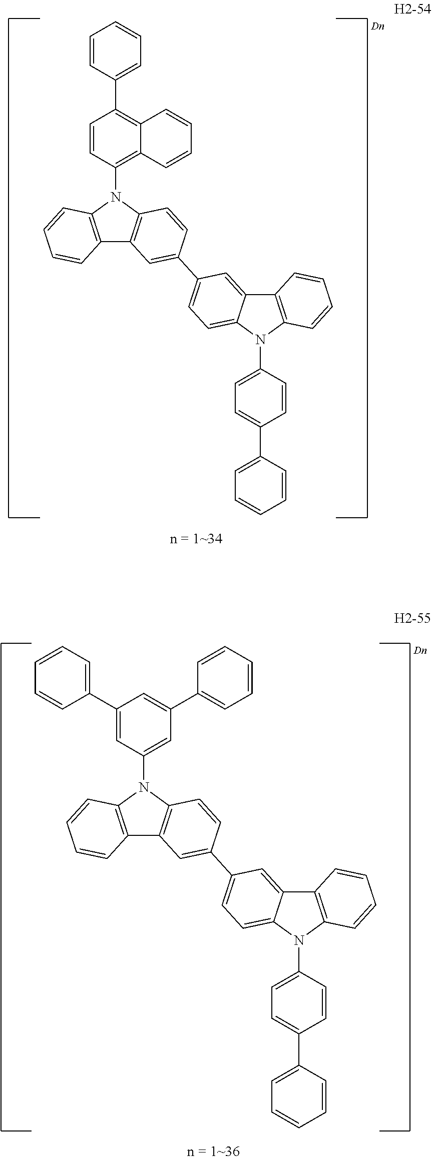

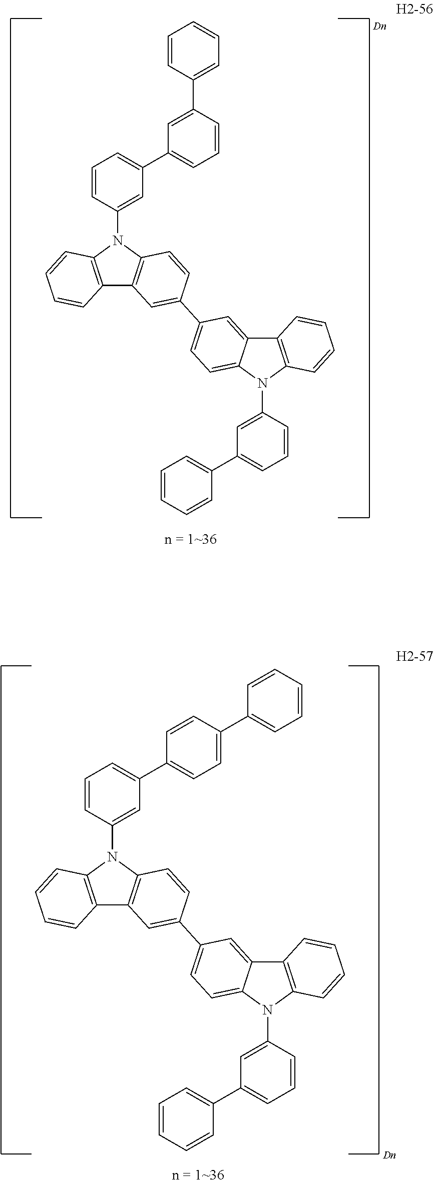

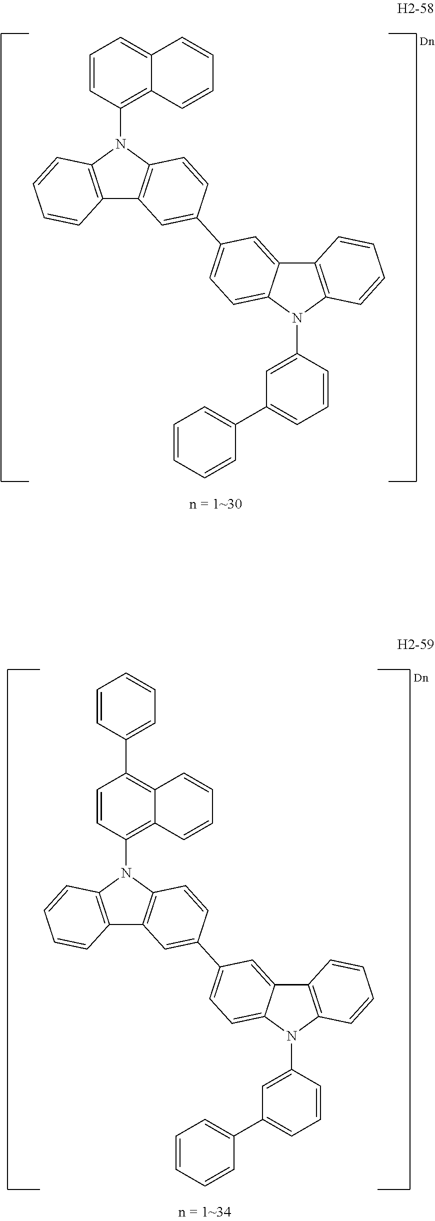

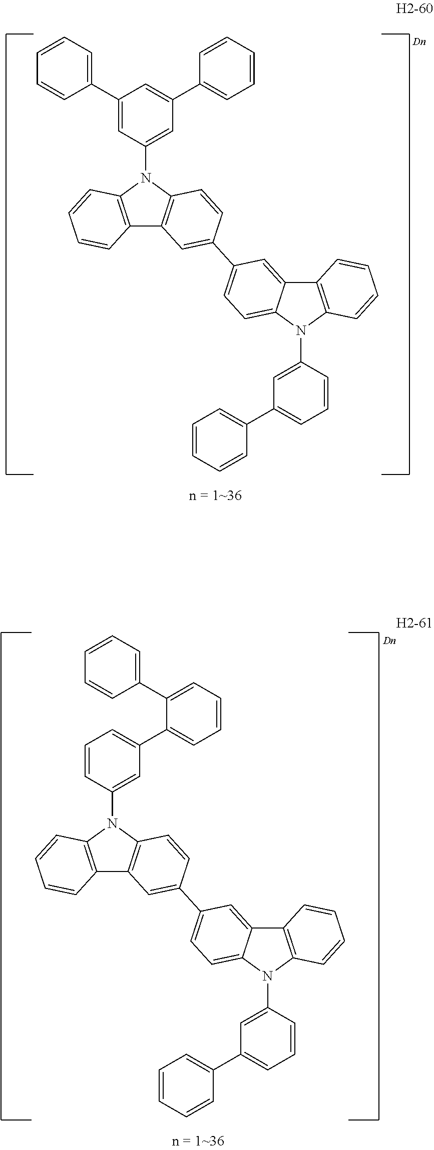

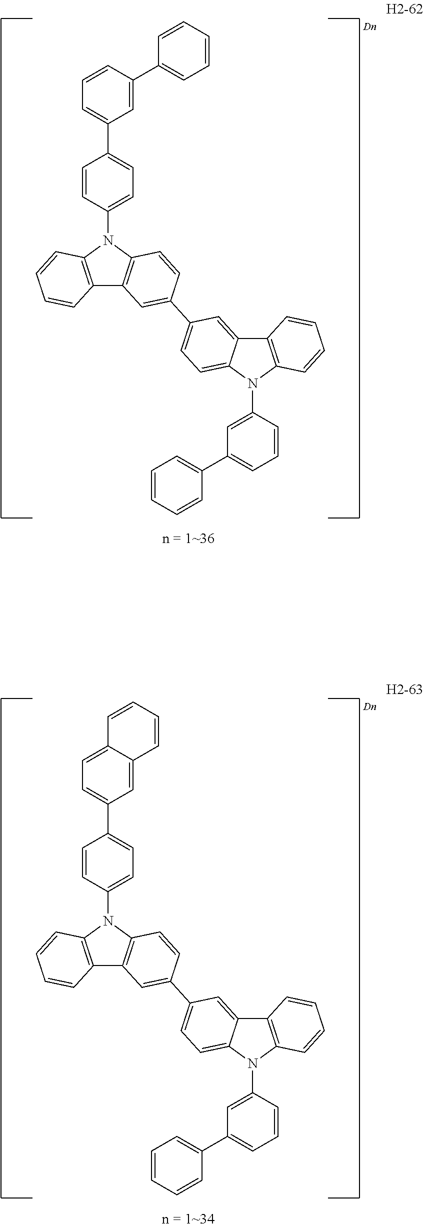

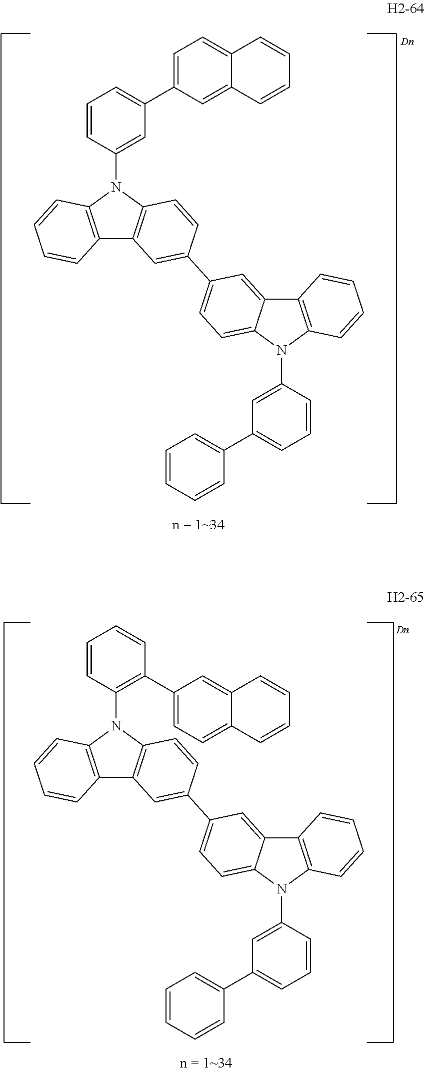

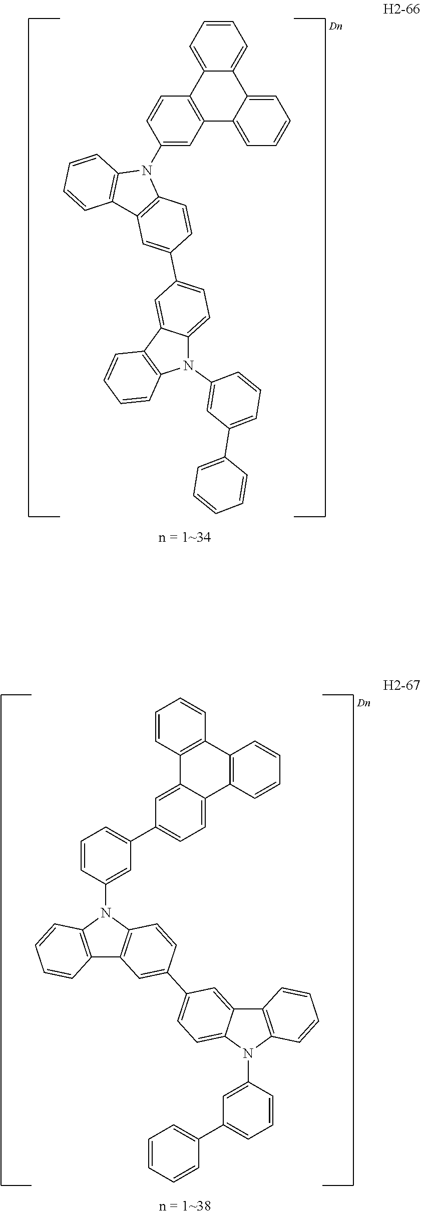

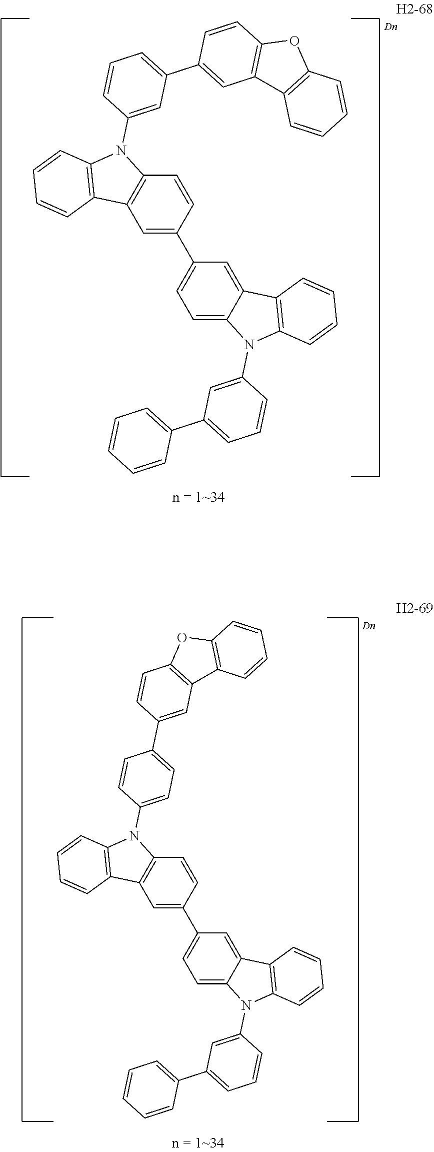

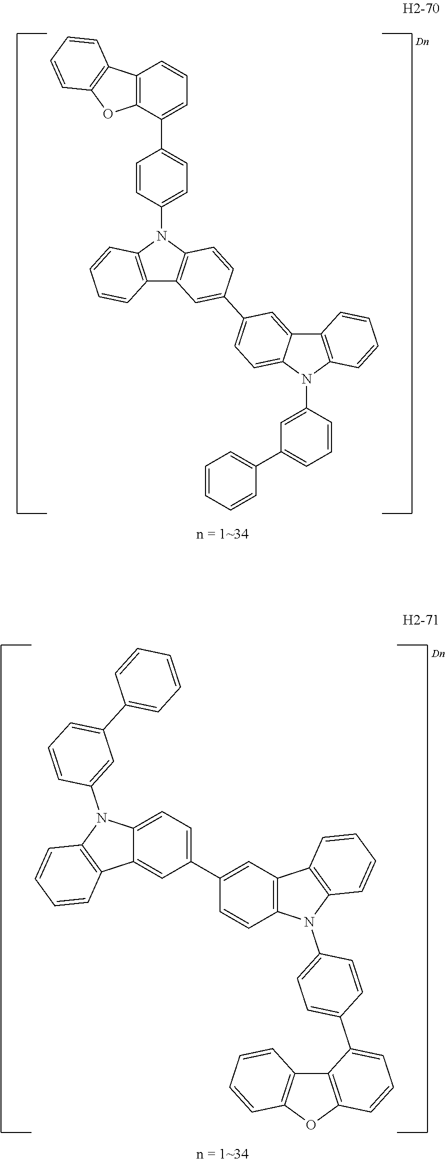

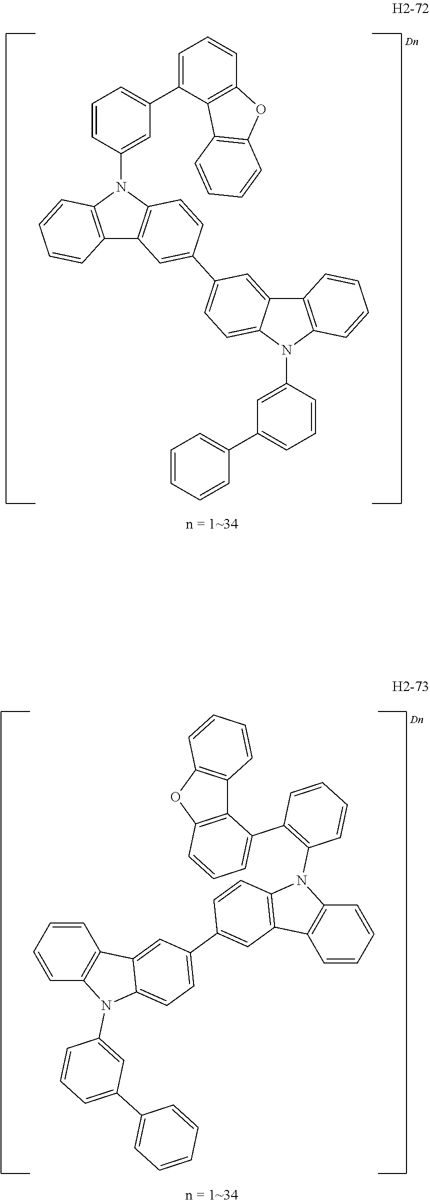

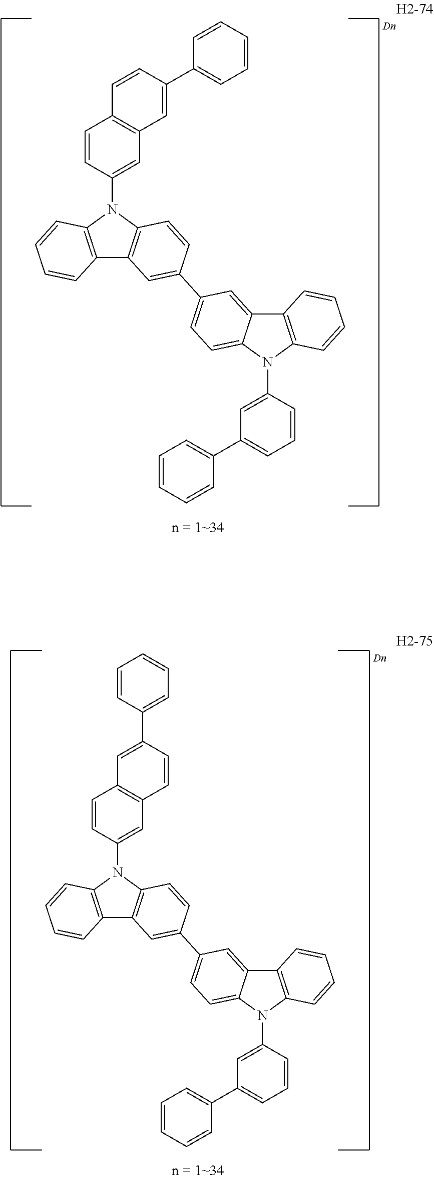

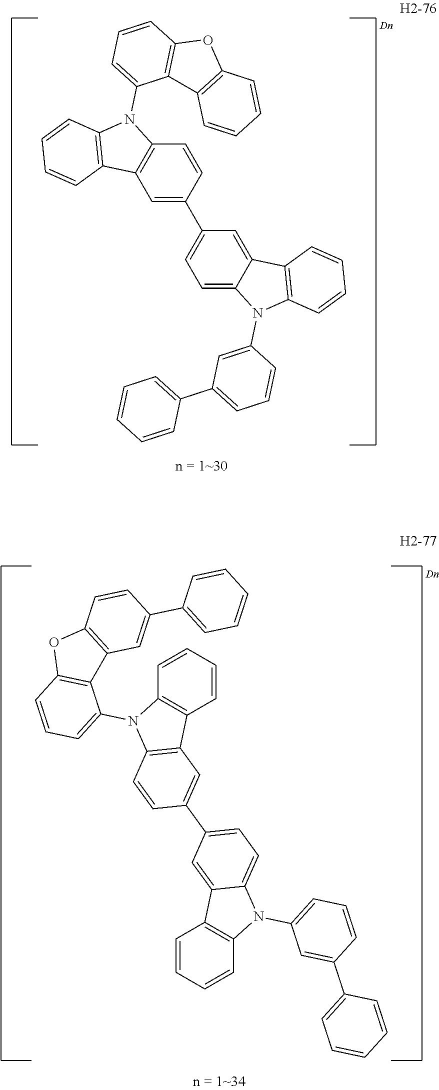

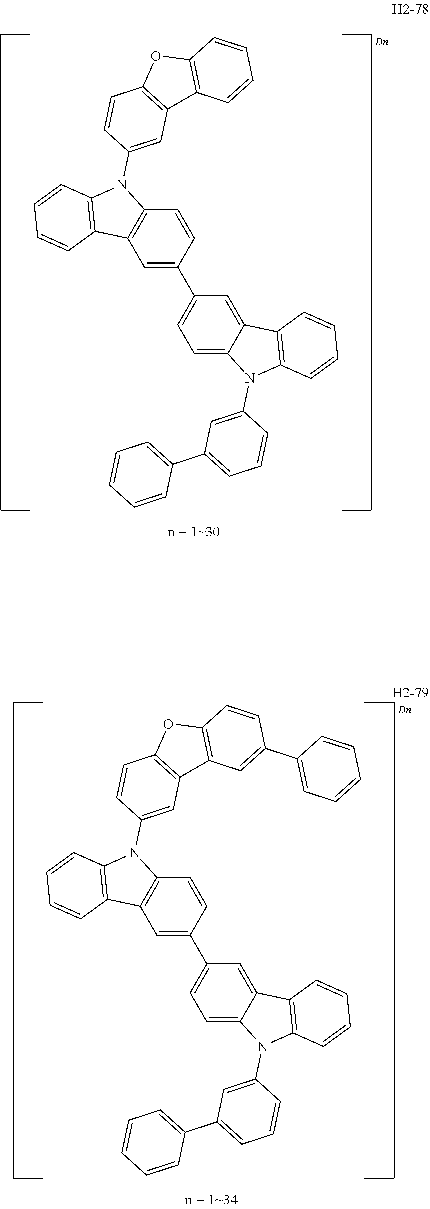

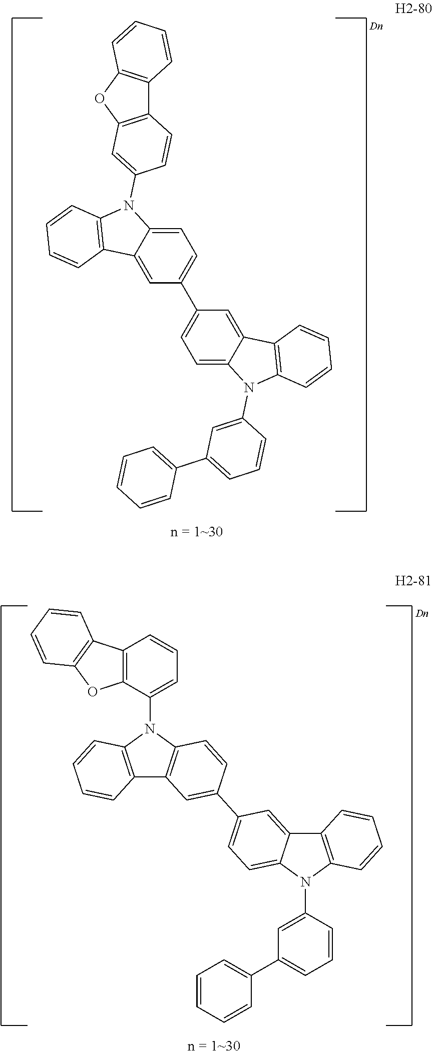

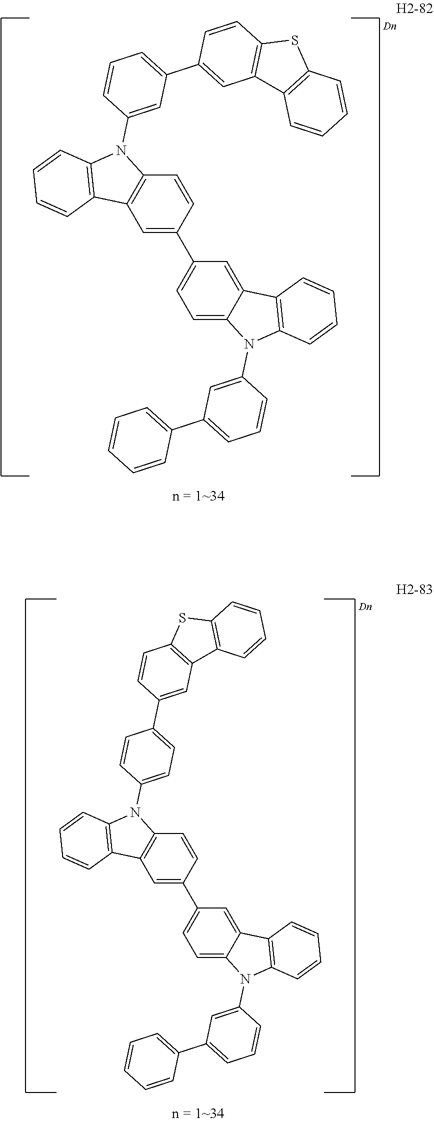

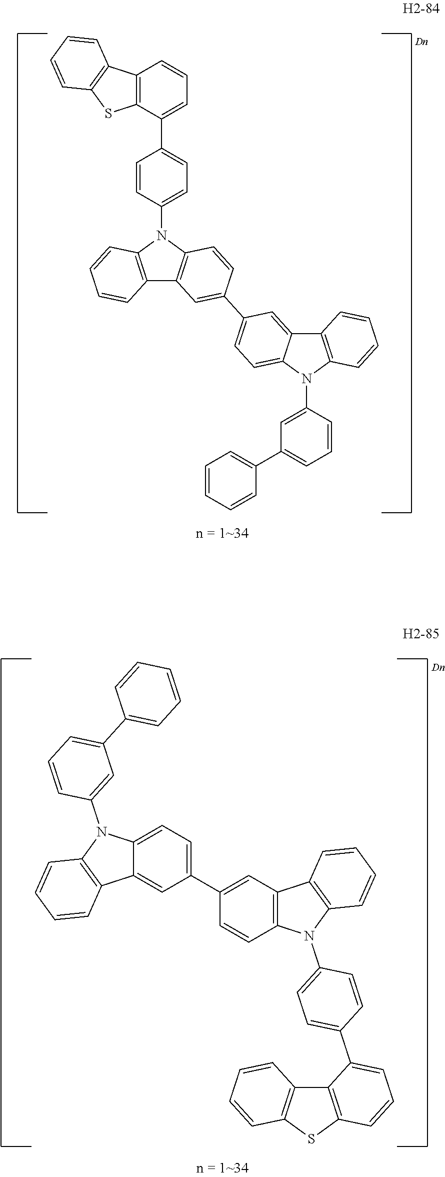

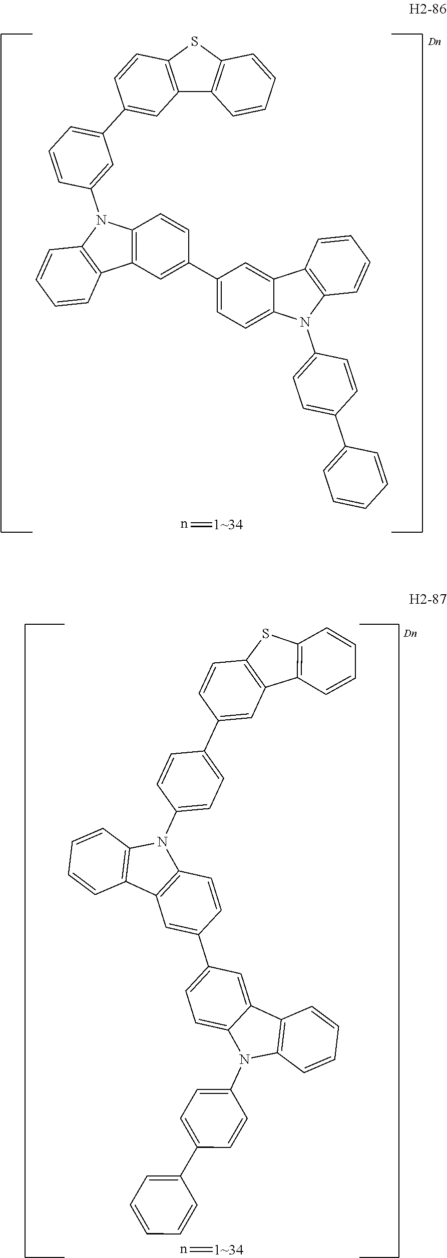

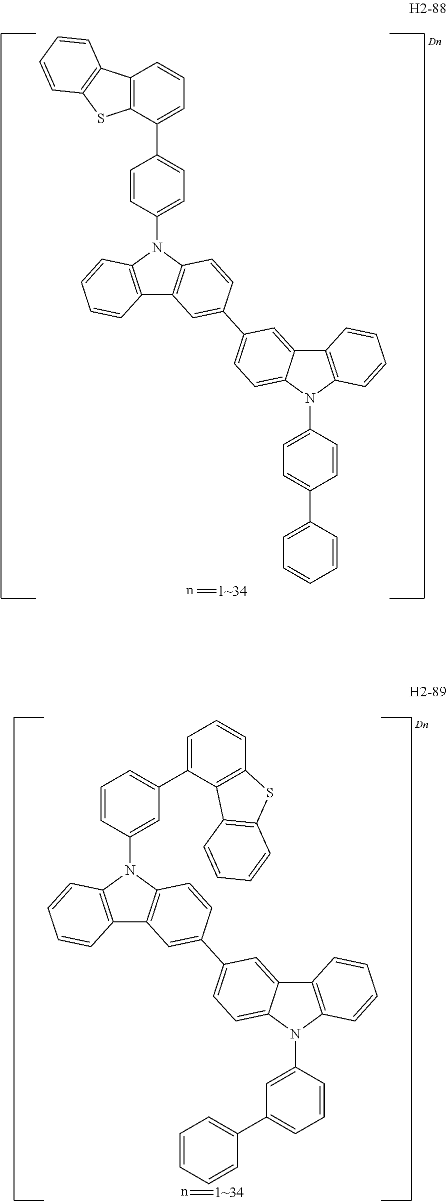

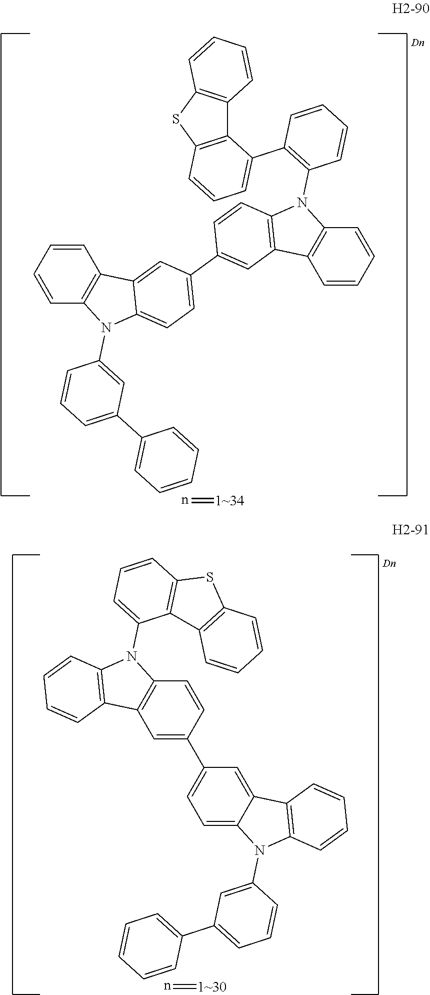

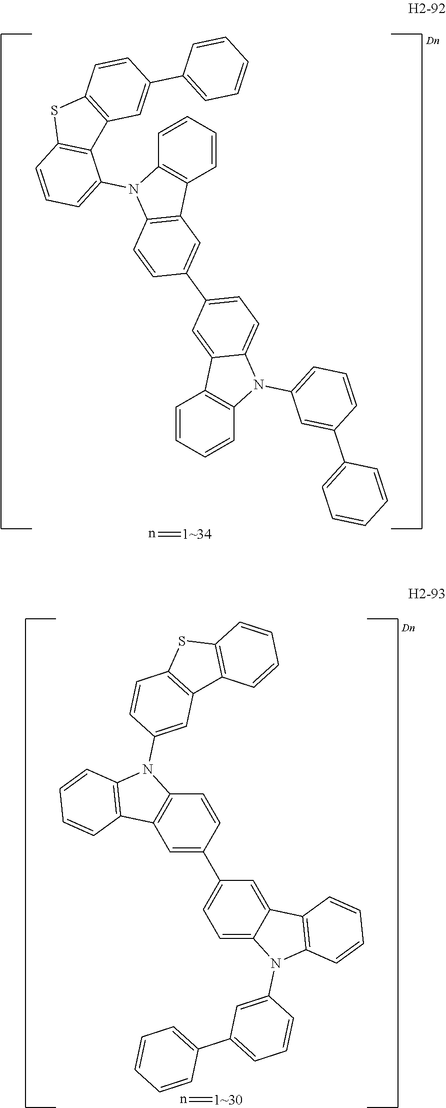

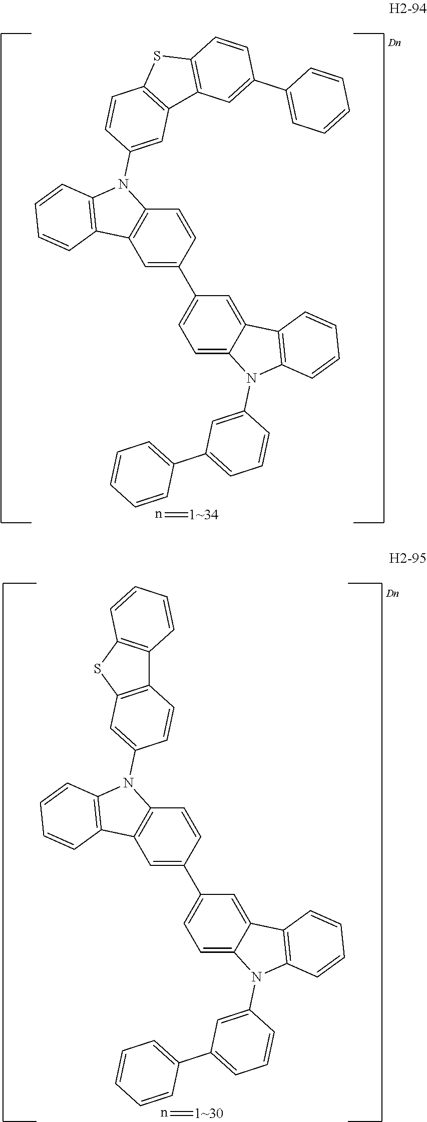

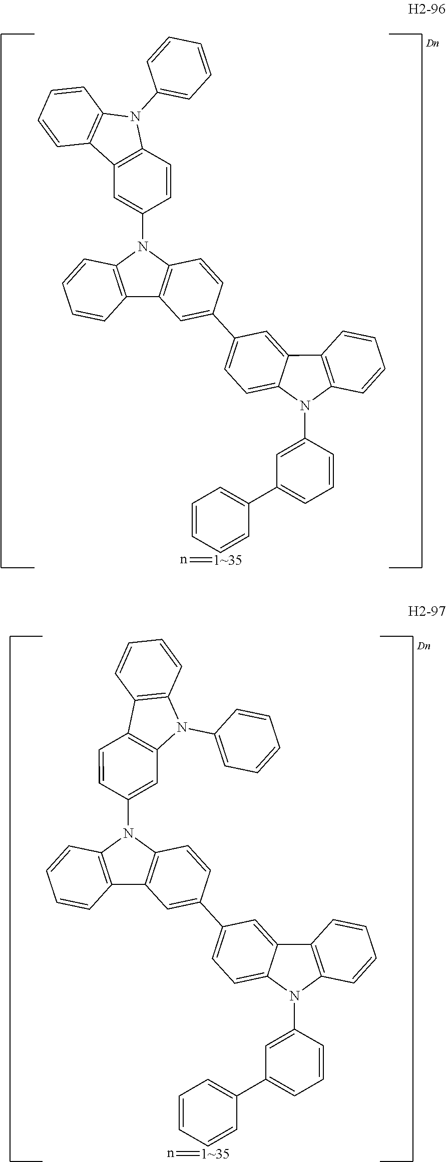

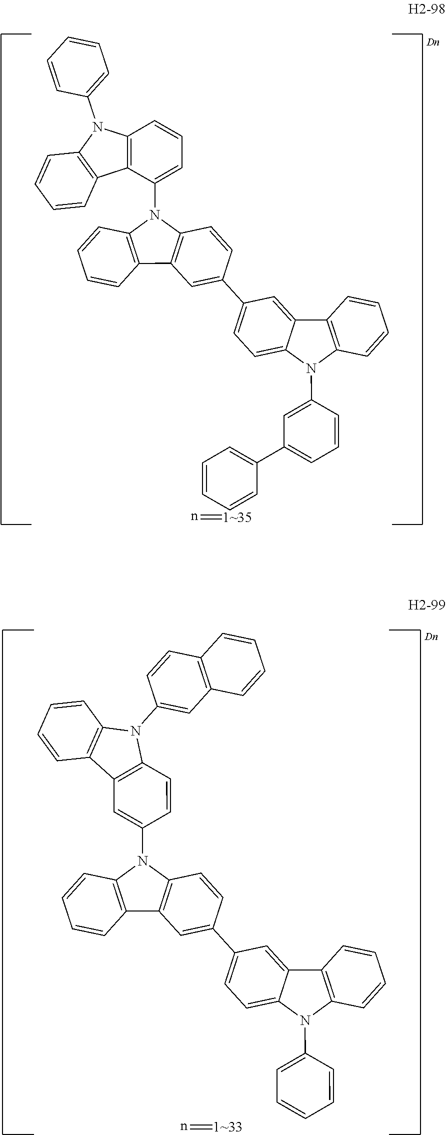

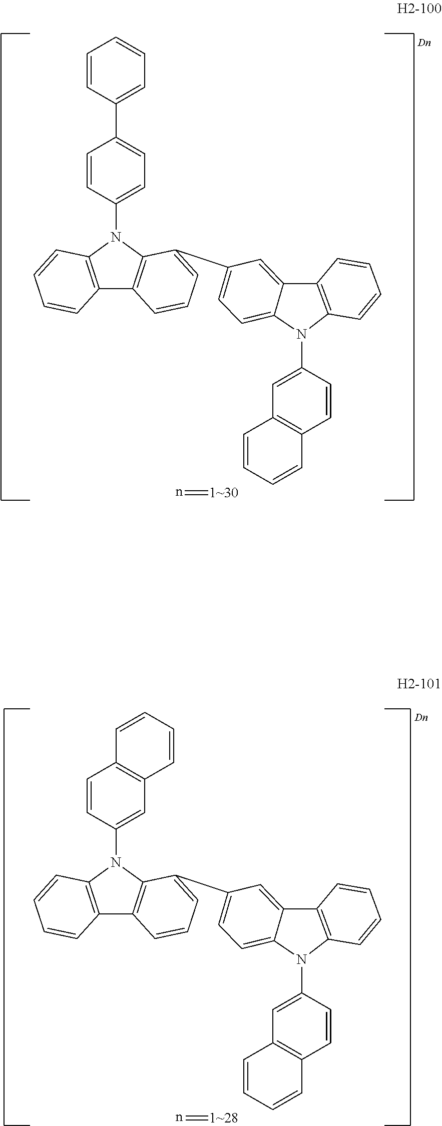

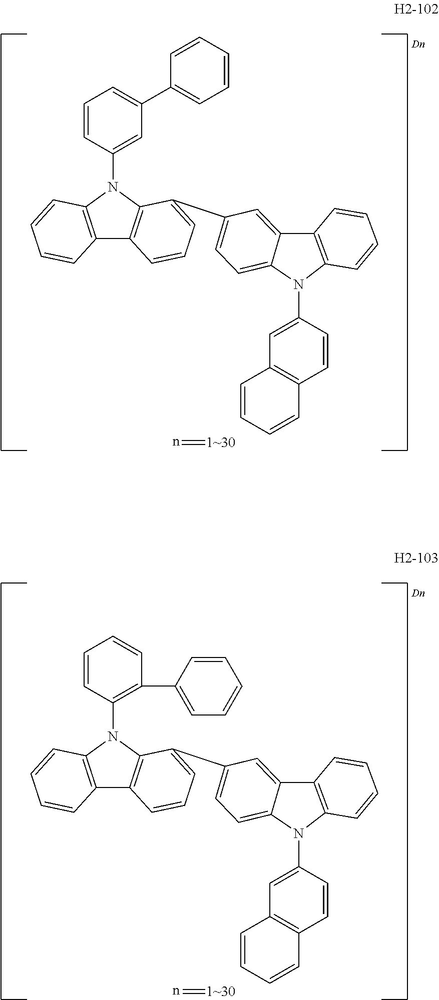

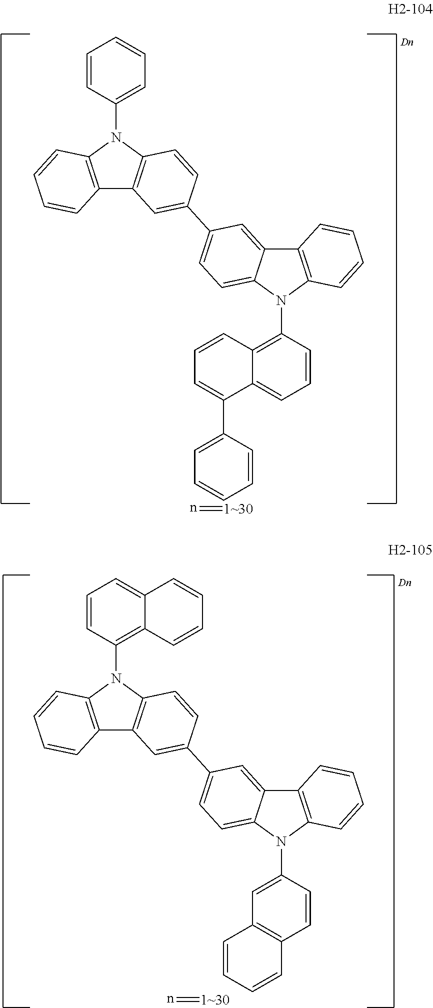

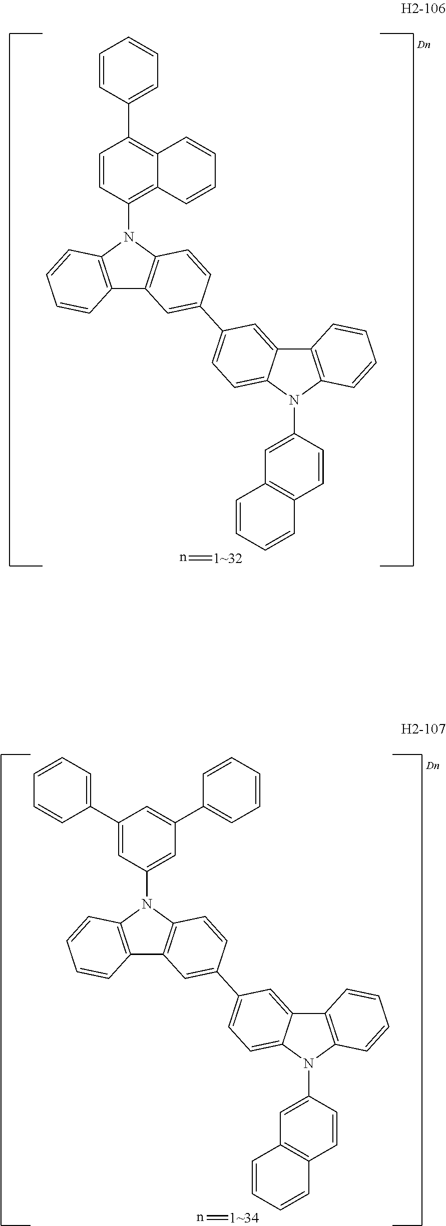

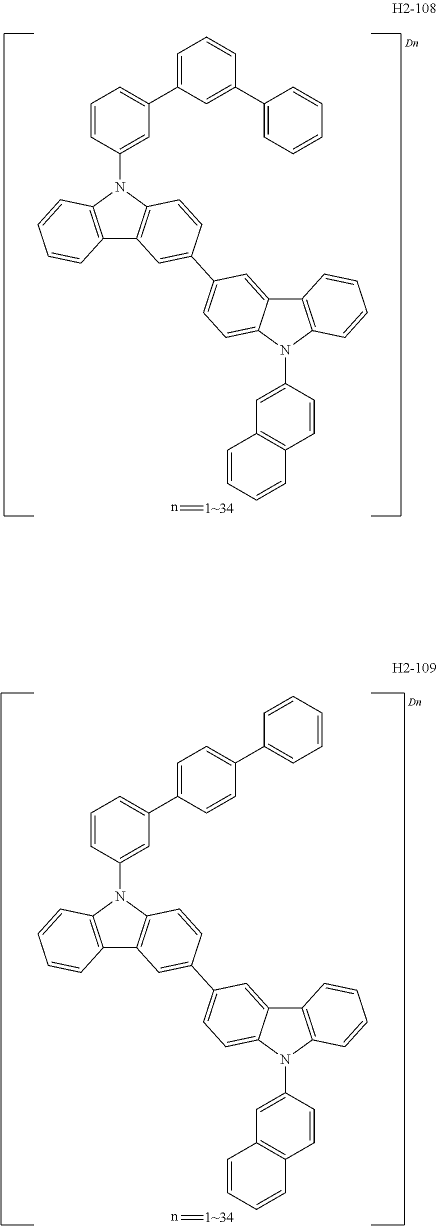

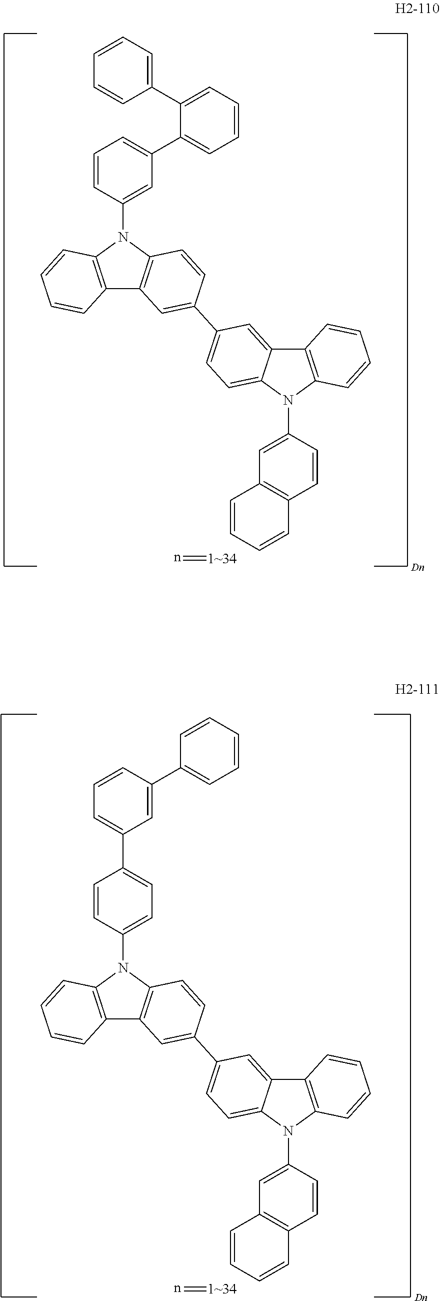

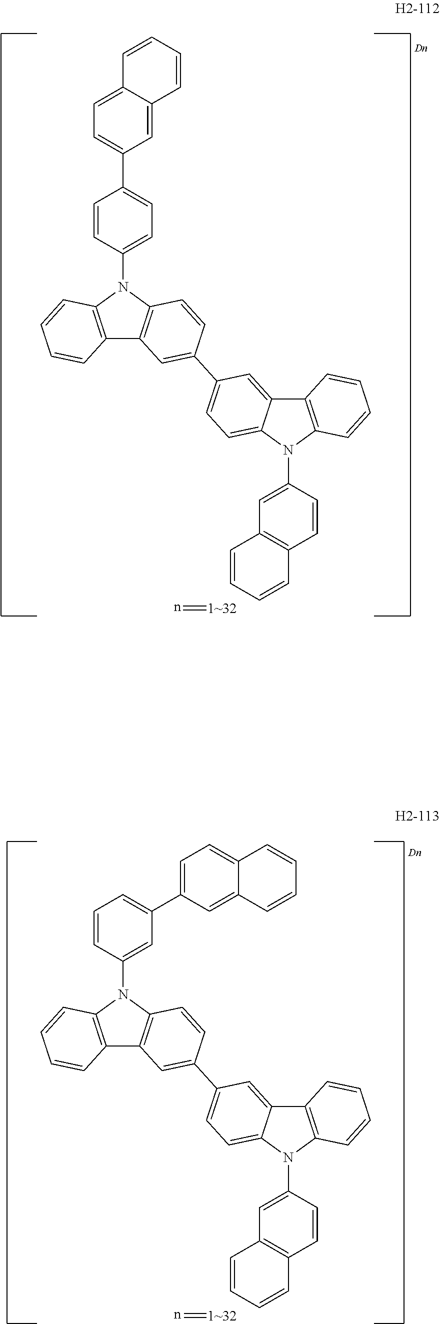

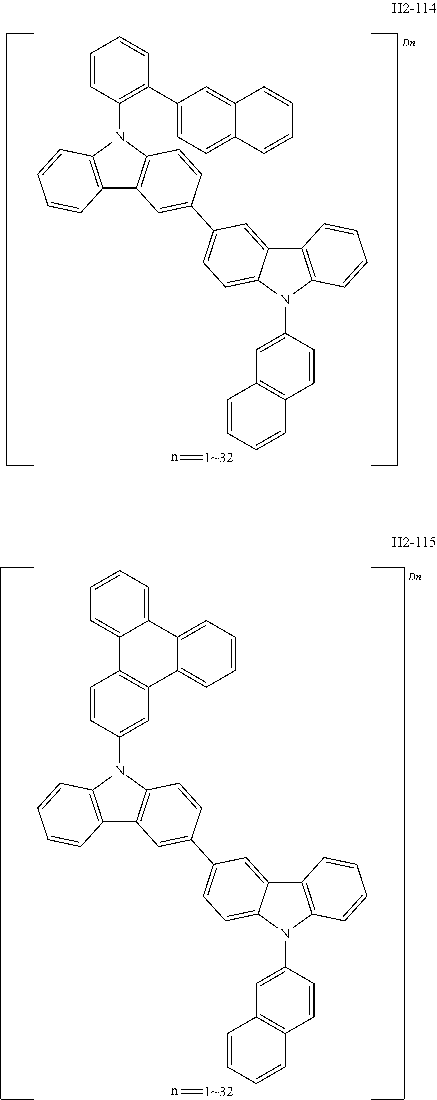

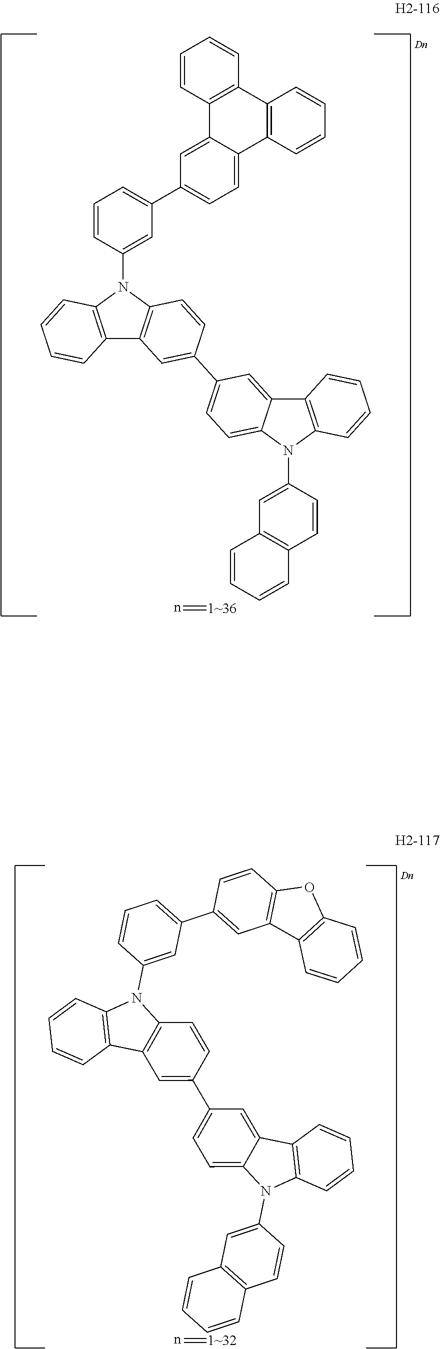

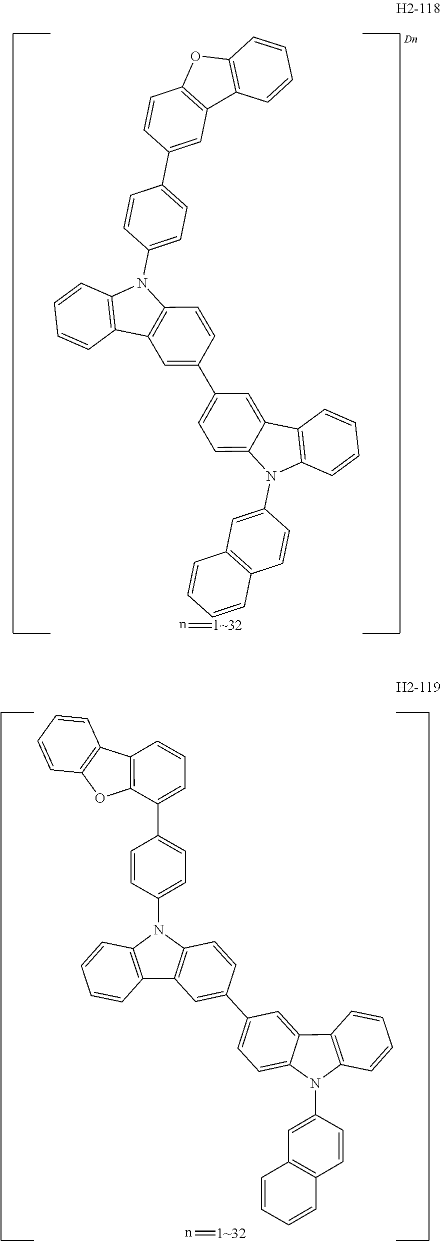

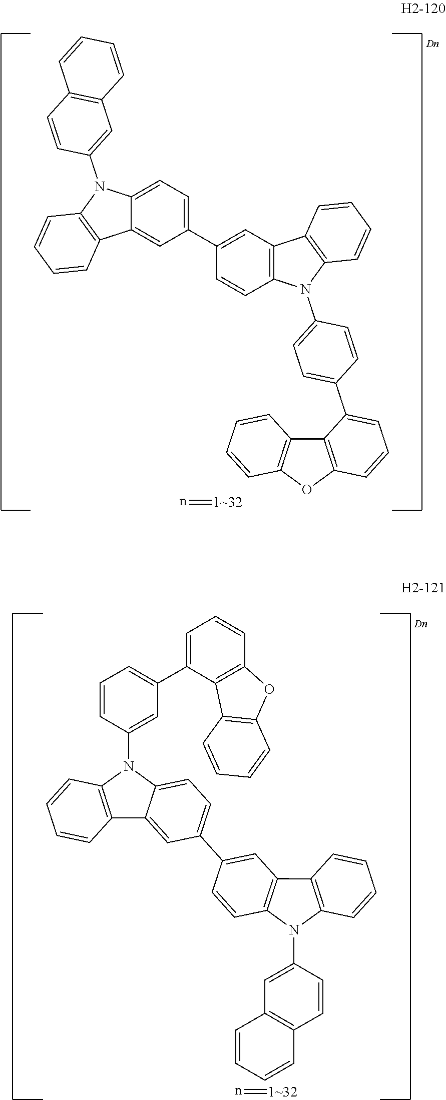

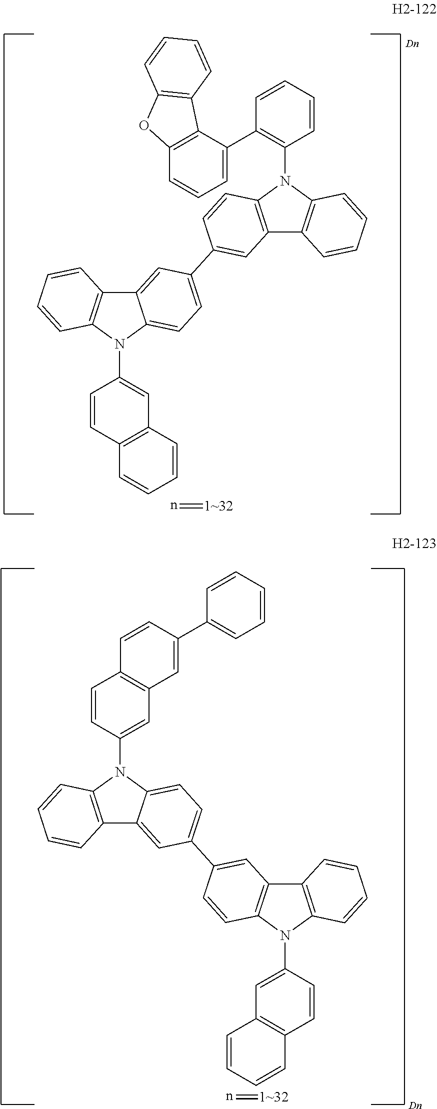

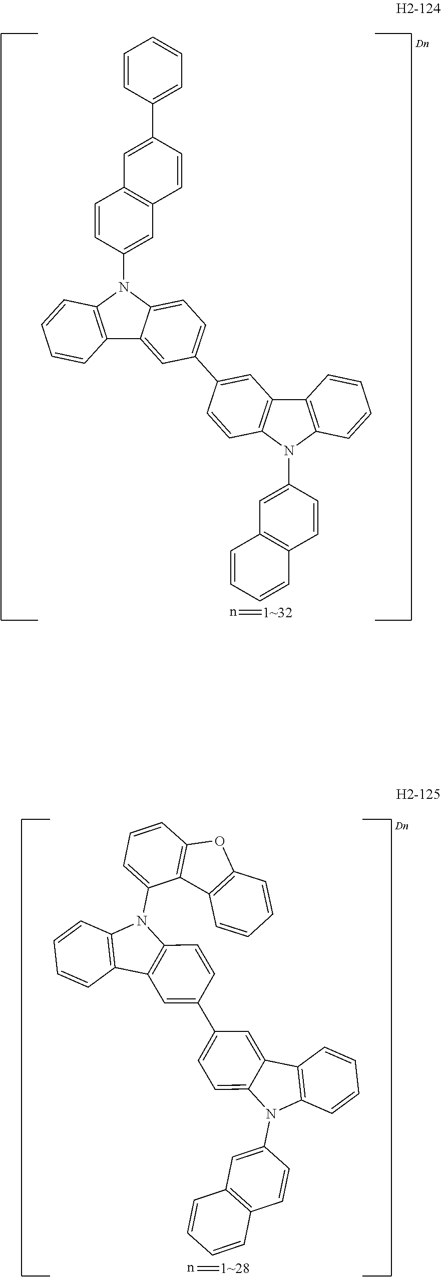

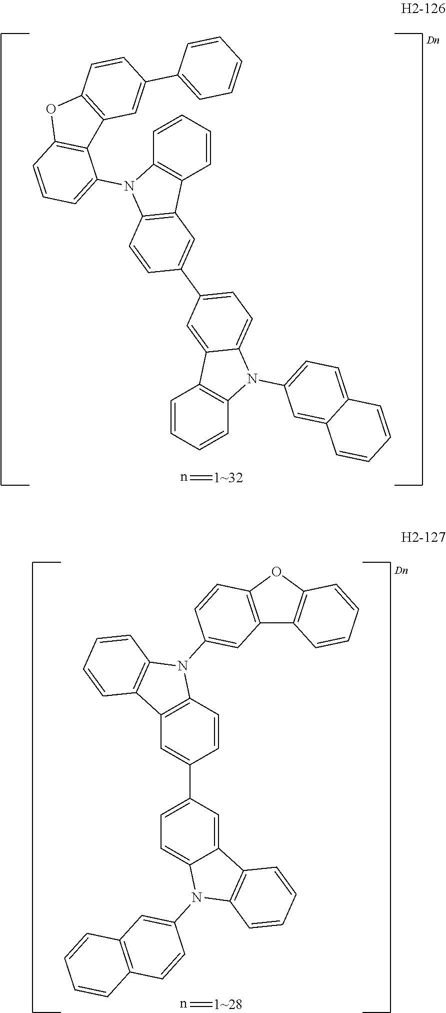

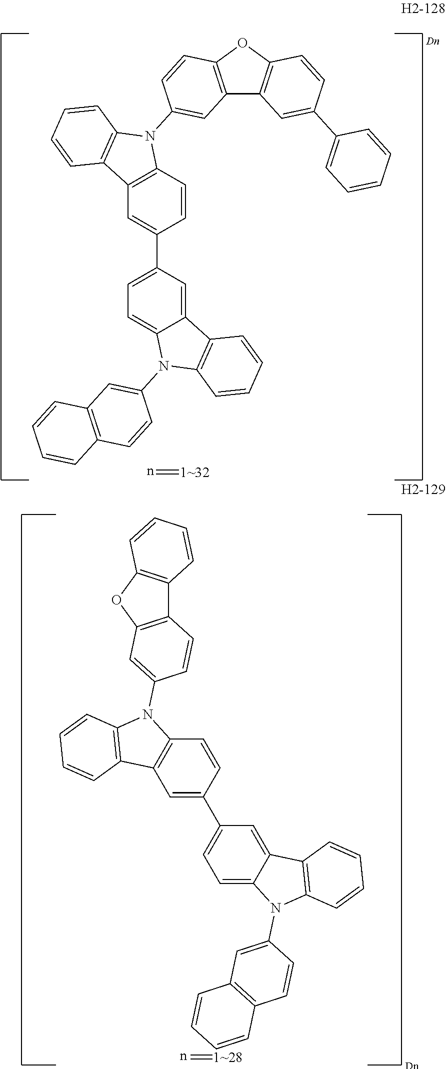

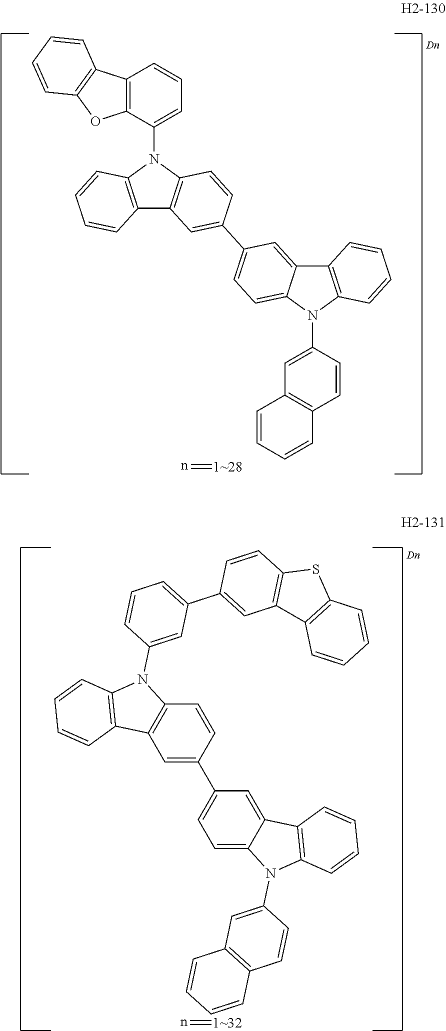

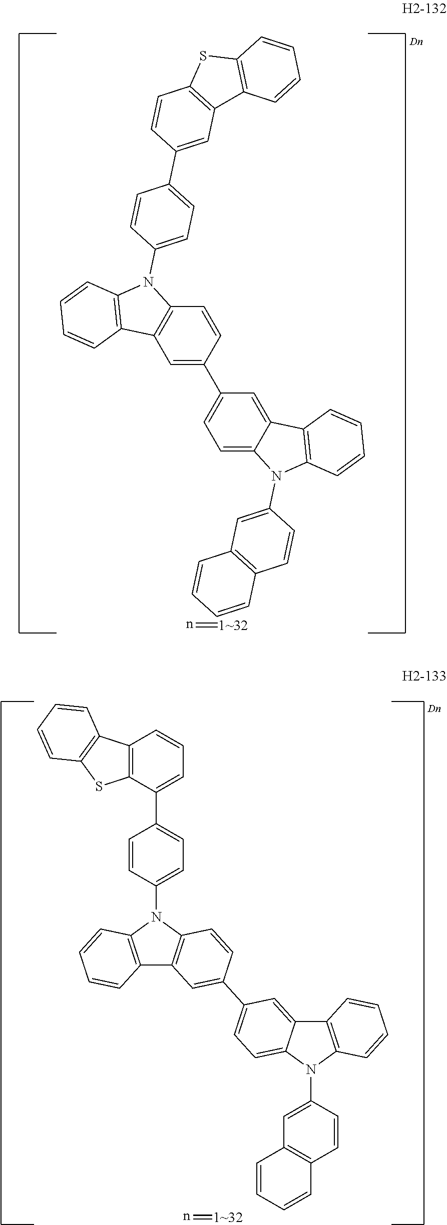

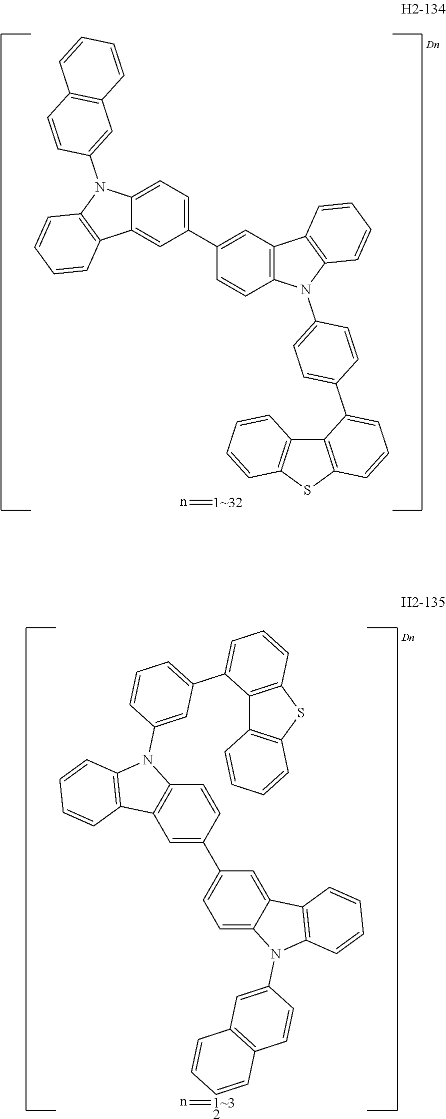

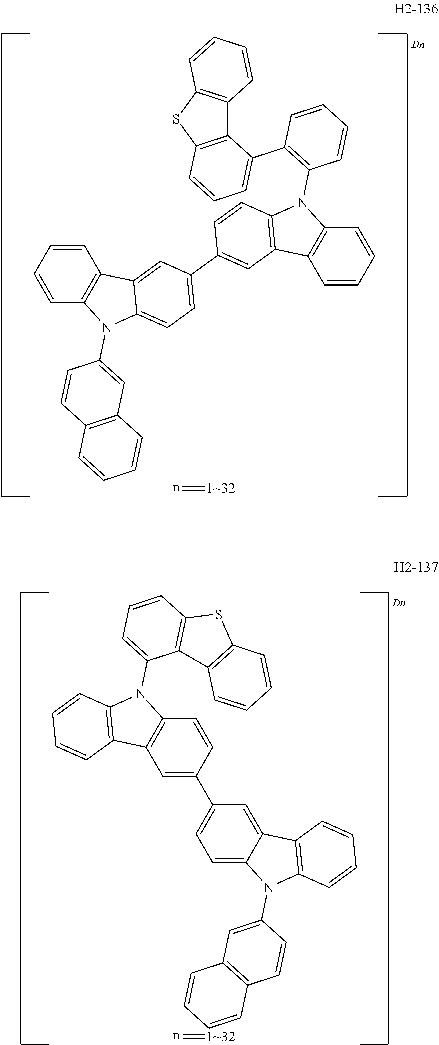

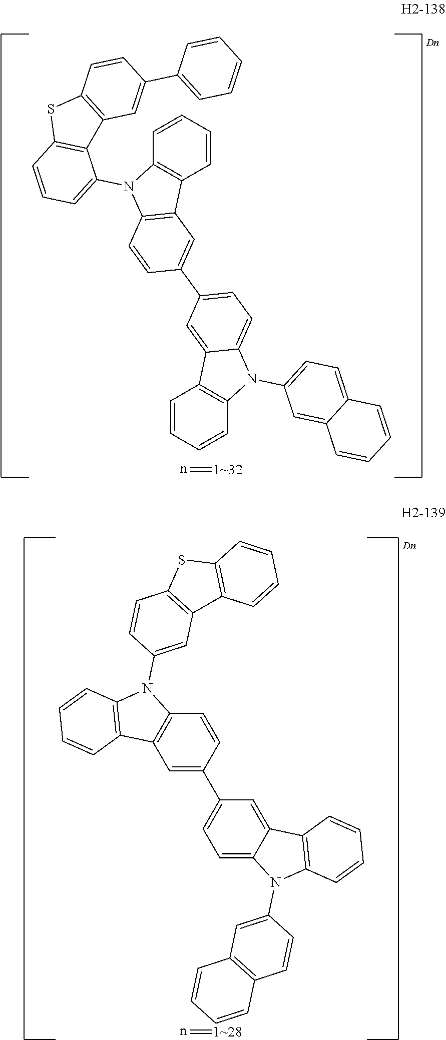

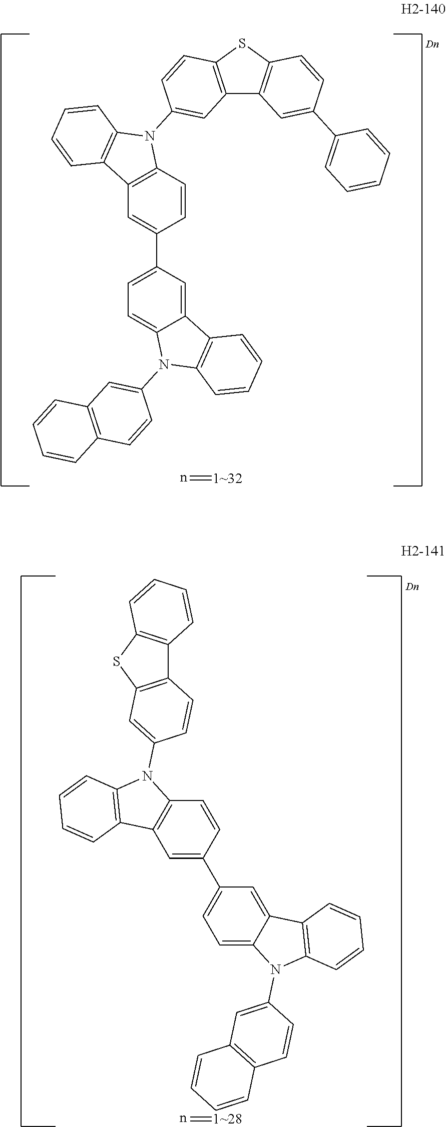

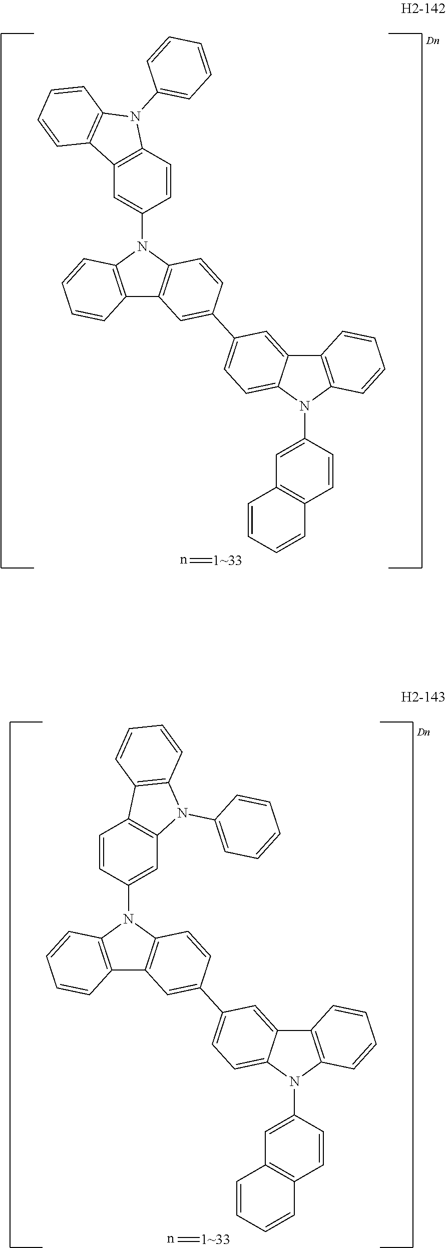

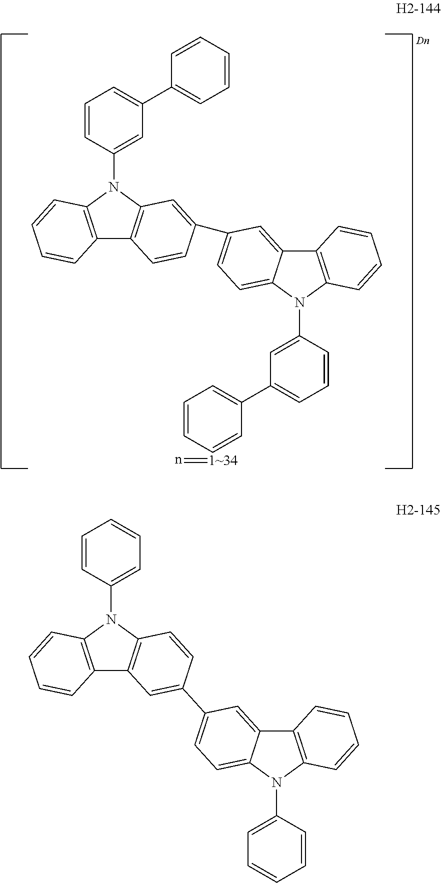

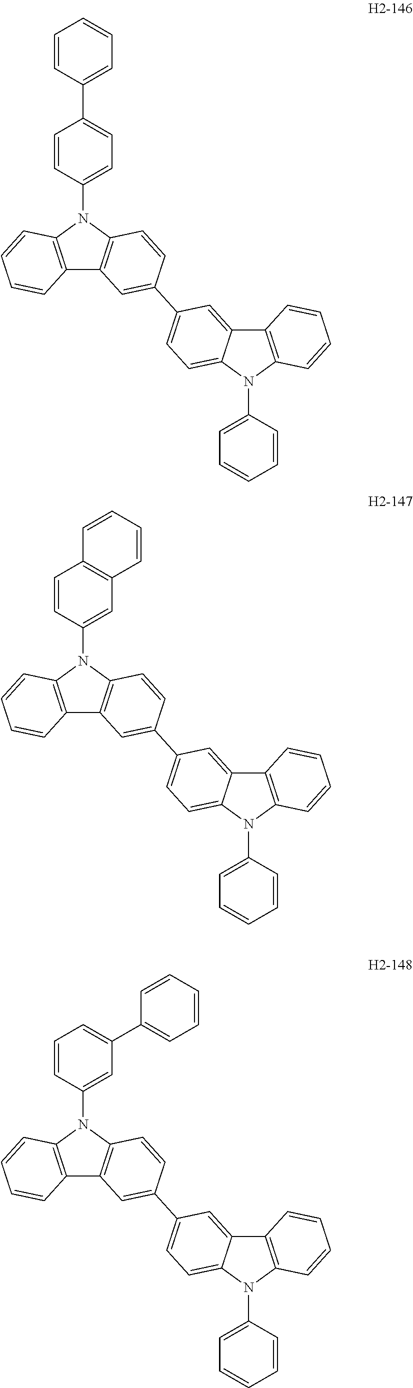 [SMILES: c1ccc(-c2ccc(-n3c4ccccc4c4cc(-c5ccc6c(c5)c5ccccc5n6-c5ccccc5)ccc43)cc2)cc1 ; c1ccc(-n2c3ccccc3c3cc(-c4ccc5c(c4)c4ccccc4n5-c4ccc5ccccc5c4)ccc32)cc1 ; c1ccc(-c2cccc(-n3c4ccccc4c4cc(-c5ccc6c(c5)c5ccccc5n6-c5ccccc5)ccc43)c2)cc1]

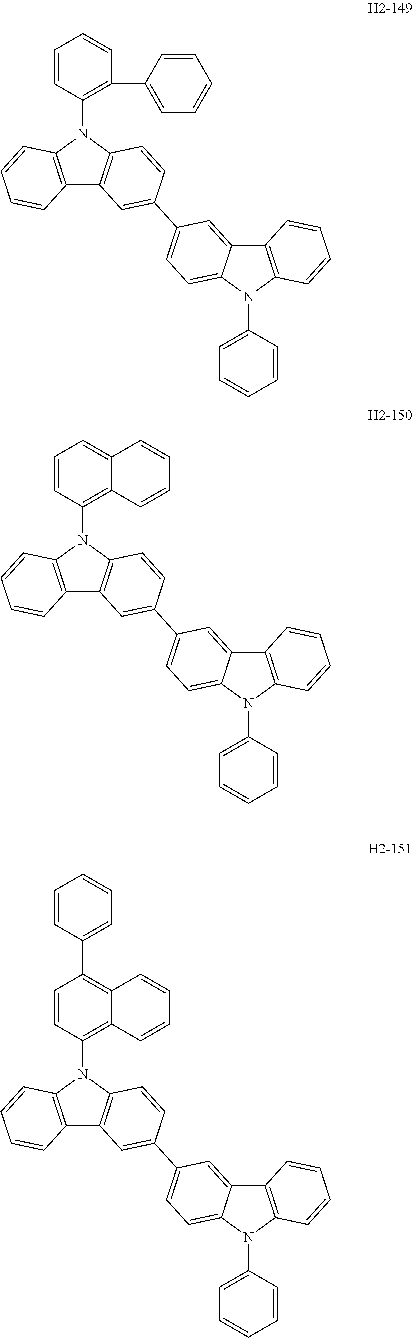 [SMILES: c1ccc(-c2ccccc2-n2c3ccccc3c3cc(-c4ccc5c(c4)c4ccccc4n5-c4ccccc4)ccc32)cc1 ; c1ccc(-n2c3ccccc3c3cc(-c4ccc5c(c4)c4ccccc4n5-c4cccc5ccccc45)ccc32)cc1 ; c1ccc(-c2ccc(-n3c4ccccc4c4cc(-c5ccc6c(c5)c5ccccc5n6-c5ccccc5)ccc43)c3ccccc23)cc1]

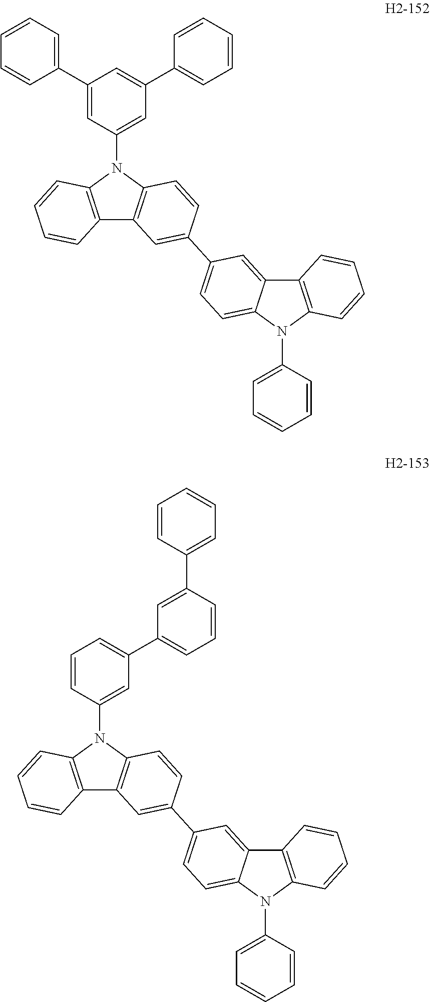 [SMILES: c1ccc(-c2cc(-c3ccccc3)cc(-n3c4ccccc4c4cc(-c5ccc6c(c5)c5ccccc5n6-c5ccccc5)ccc43)c2)cc1 ; c1ccc(-c2cccc(-c3cccc(-n4c5ccccc5c5cc(-c6ccc7c(c6)c6ccccc6n7-c6ccccc6)ccc54)c3)c2)cc1]

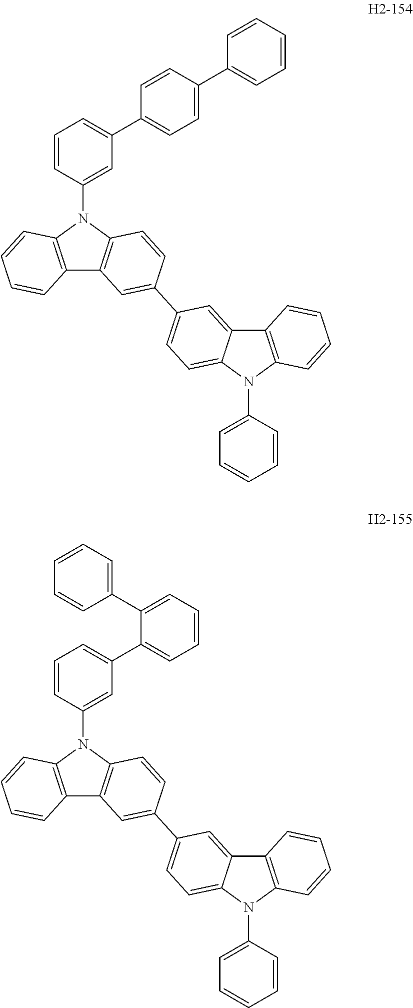 [SMILES: c1ccc(-c2ccc(-c3cccc(-n4c5ccccc5c5cc(-c6ccc7c(c6)c6ccccc6n7-c6ccccc6)ccc54)c3)cc2)cc1 ; c1ccc(-c2ccccc2-c2cccc(-n3c4ccccc4c4cc(-c5ccc6c(c5)c5ccccc5n6-c5ccccc5)ccc43)c2)cc1]

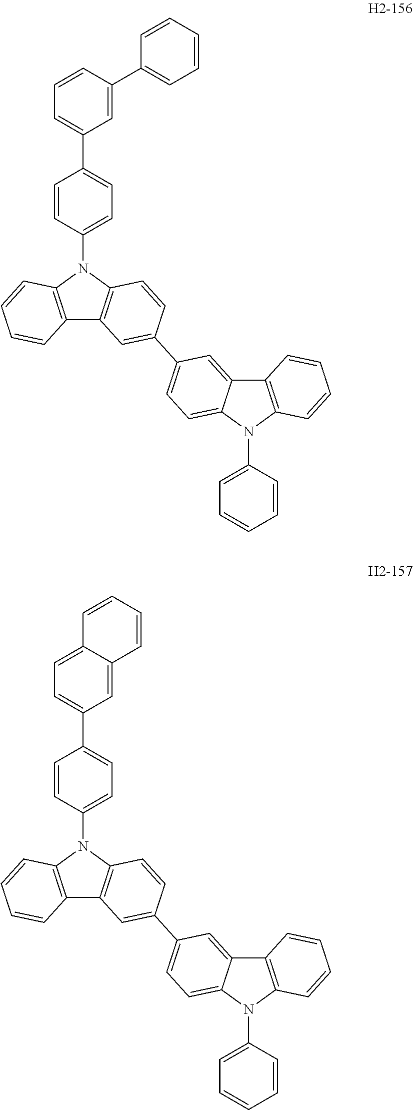 [SMILES: c1ccc(-c2cccc(-c3ccc(-n4c5ccccc5c5cc(-c6ccc7c(c6)c6ccccc6n7-c6ccccc6)ccc54)cc3)c2)cc1 ; c1ccc(-n2c3ccccc3c3cc(-c4ccc5c(c4)c4ccccc4n5-c4ccc(-c5ccc6ccccc6c5)cc4)ccc32)cc1]

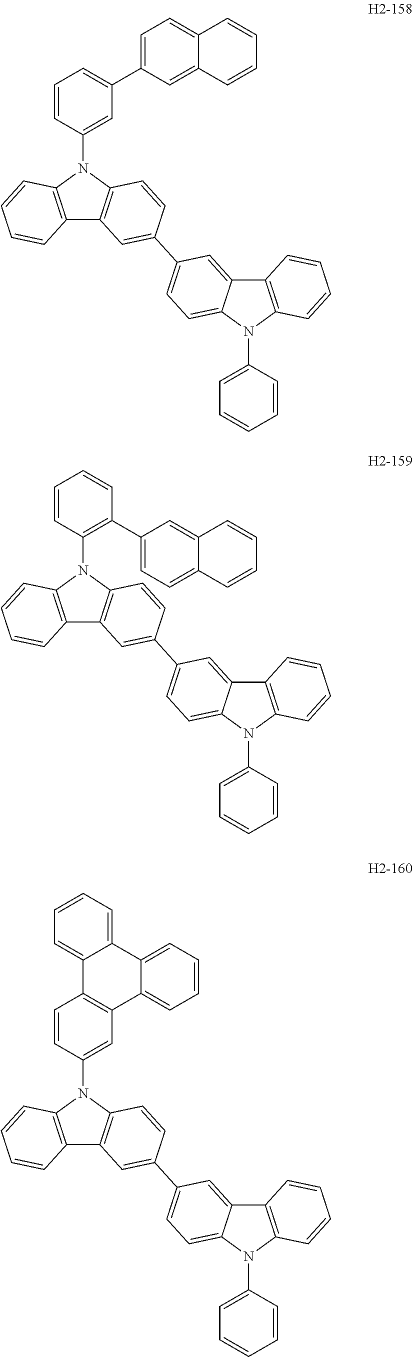 [SMILES: c1ccc(-n2c3ccccc3c3cc(-c4ccc5c(c4)c4ccccc4n5-c4cccc(-c5ccc6ccccc6c5)c4)ccc32)cc1 ; c1ccc(-n2c3ccccc3c3cc(-c4ccc5c(c4)c4ccccc4n5-c4ccccc4-c4ccc5ccccc5c4)ccc32)cc1 ; c1ccc(-n2c3ccccc3c3cc(-c4ccc5c(c4)c4ccccc4n5-c4ccc5c6ccccc6c6ccccc6c5c4)ccc32)cc1]

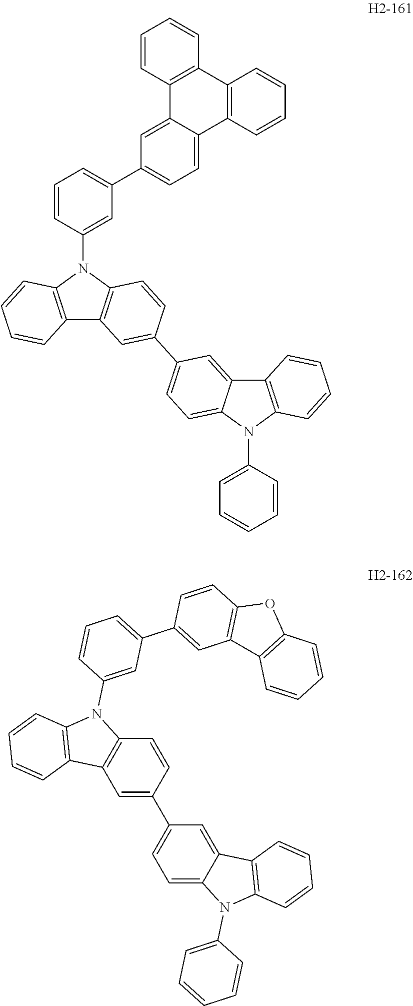 [SMILES: c1ccc(-n2c3ccccc3c3cc(-c4ccc5c(c4)c4ccccc4n5-c4cccc(-c5ccc6c7ccccc7c7ccccc7c6c5)c4)ccc32)cc1 ; c1ccc(-n2c3ccccc3c3cc(-c4ccc5c(c4)c4ccccc4n5-c4cccc(-c5ccc6oc7ccccc7c6c5)c4)ccc32)cc1]

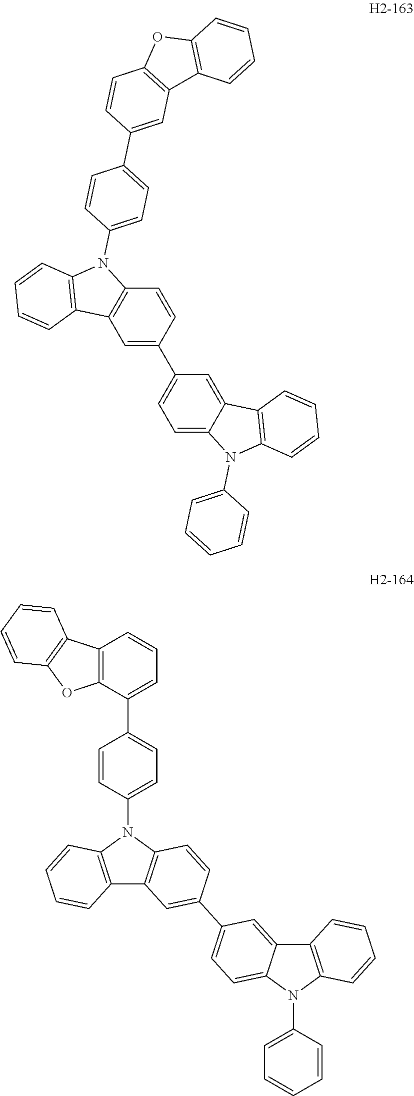 [SMILES: c1ccc(-n2c3ccccc3c3cc(-c4ccc5c(c4)c4ccccc4n5-c4ccc(-c5ccc6oc7ccccc7c6c5)cc4)ccc32)cc1 ; c1ccc(-n2c3ccccc3c3cc(-c4ccc5c(c4)c4ccccc4n5-c4ccc(-c5cccc6c5oc5ccccc56)cc4)ccc32)cc1]

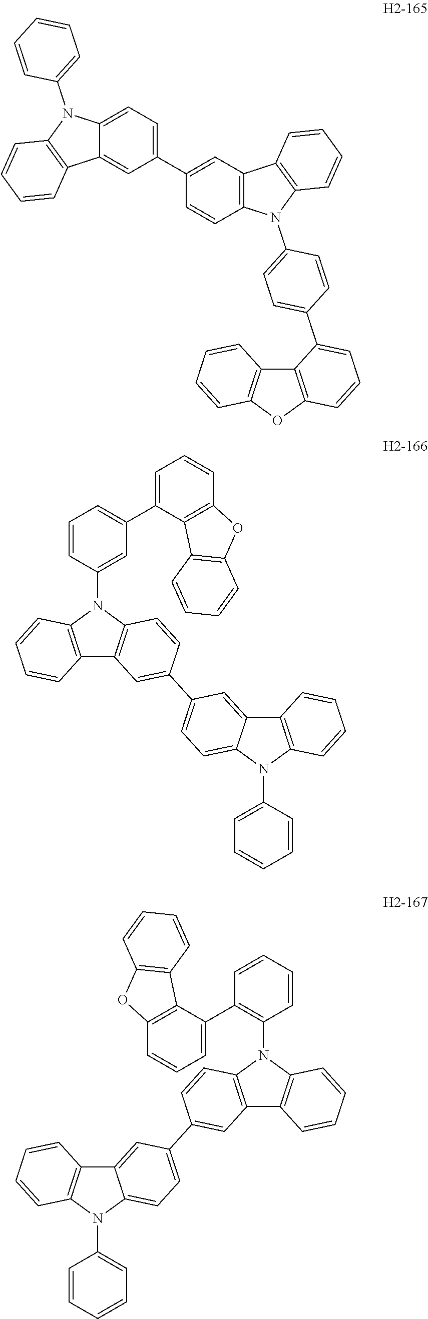 [SMILES: c1ccc(-n2c3ccccc3c3cc(-c4ccc5c(c4)c4ccccc4n5-c4ccc(-c5cccc6oc7ccccc7c56)cc4)ccc32)cc1 ; c1ccc(-n2c3ccccc3c3cc(-c4ccc5c(c4)c4ccccc4n5-c4cccc(-c5cccc6oc7ccccc7c56)c4)ccc32)cc1 ; c1ccc(-n2c3ccccc3c3cc(-c4ccc5c(c4)c4ccccc4n5-c4ccccc4-c4cccc5oc6ccccc6c45)ccc32)cc1]

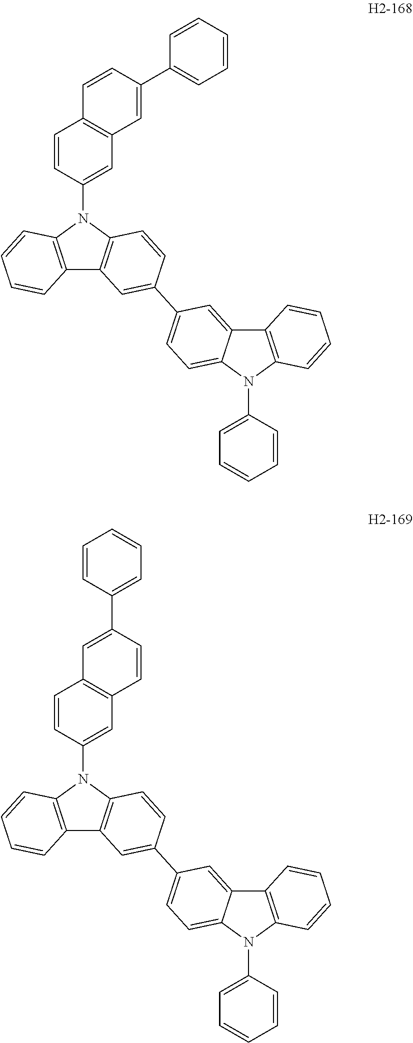 [SMILES: c1ccc(-c2ccc3ccc(-n4c5ccccc5c5cc(-c6ccc7c(c6)c6ccccc6n7-c6ccccc6)ccc54)cc3c2)cc1 ; c1ccc(-c2ccc3cc(-n4c5ccccc5c5cc(-c6ccc7c(c6)c6ccccc6n7-c6ccccc6)ccc54)ccc3c2)cc1]

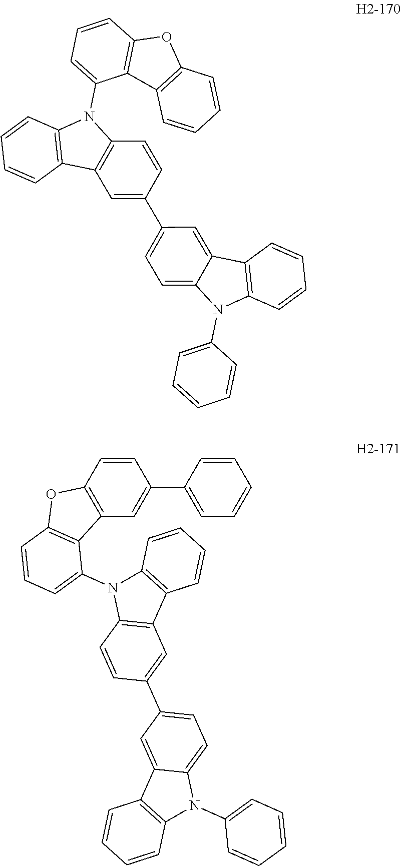 [SMILES: c1ccc(-n2c3ccccc3c3cc(-c4ccc5c(c4)c4ccccc4n5-c4cccc5oc6ccccc6c45)ccc32)cc1 ; c1ccc(-c2ccc3oc4cccc(-n5c6ccccc6c6cc(-c7ccc8c(c7)c7ccccc7n8-c7ccccc7)ccc65)c4c3c2)cc1]

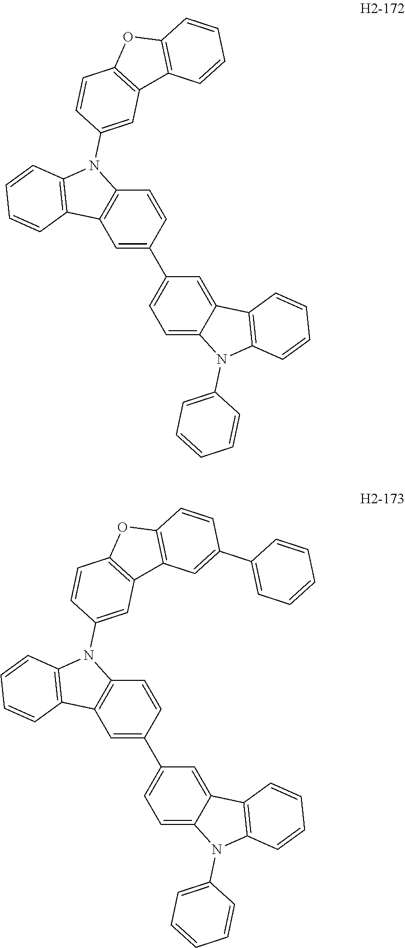 [SMILES: c1ccc(-n2c3ccccc3c3cc(-c4ccc5c(c4)c4ccccc4n5-c4ccc5oc6ccccc6c5c4)ccc32)cc1 ; c1ccc(-c2ccc3oc4ccc(-n5c6ccccc6c6cc(-c7ccc8c(c7)c7ccccc7n8-c7ccccc7)ccc65)cc4c3c2)cc1]

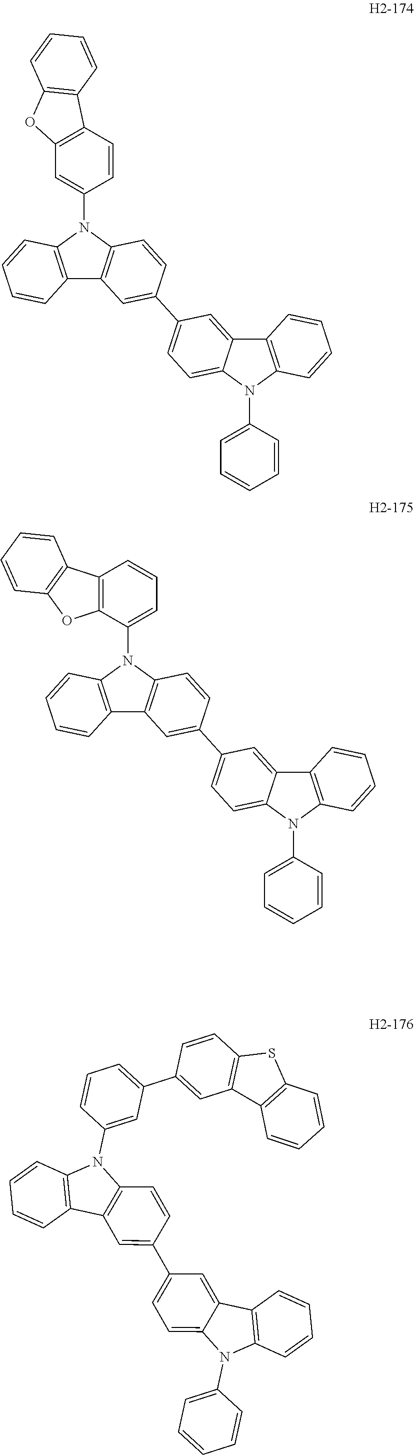 [SMILES: c1ccc(-n2c3ccccc3c3cc(-c4ccc5c(c4)c4ccccc4n5-c4ccc5c(c4)oc4ccccc45)ccc32)cc1 ; c1ccc(-n2c3ccccc3c3cc(-c4ccc5c(c4)c4ccccc4n5-c4cccc5c4oc4ccccc45)ccc32)cc1 ; c1ccc(-n2c3ccccc3c3cc(-c4ccc5c(c4)c4ccccc4n5-c4cccc(-c5ccc6sc7ccccc7c6c5)c4)ccc32)cc1]

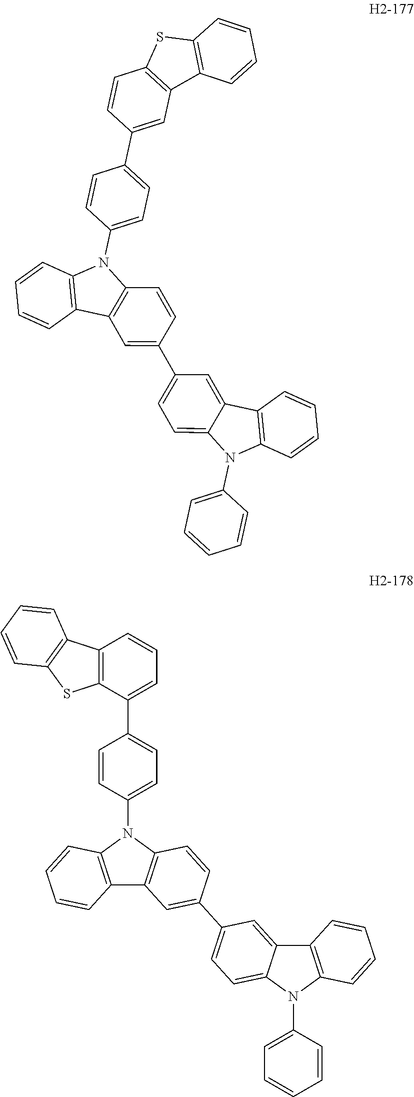 [SMILES: c1ccc(-n2c3ccccc3c3cc(-c4ccc5c(c4)c4ccccc4n5-c4ccc(-c5ccc6sc7ccccc7c6c5)cc4)ccc32)cc1 ; c1ccc(-n2c3ccccc3c3cc(-c4ccc5c(c4)c4ccccc4n5-c4ccc(-c5cccc6c5sc5ccccc56)cc4)ccc32)cc1]

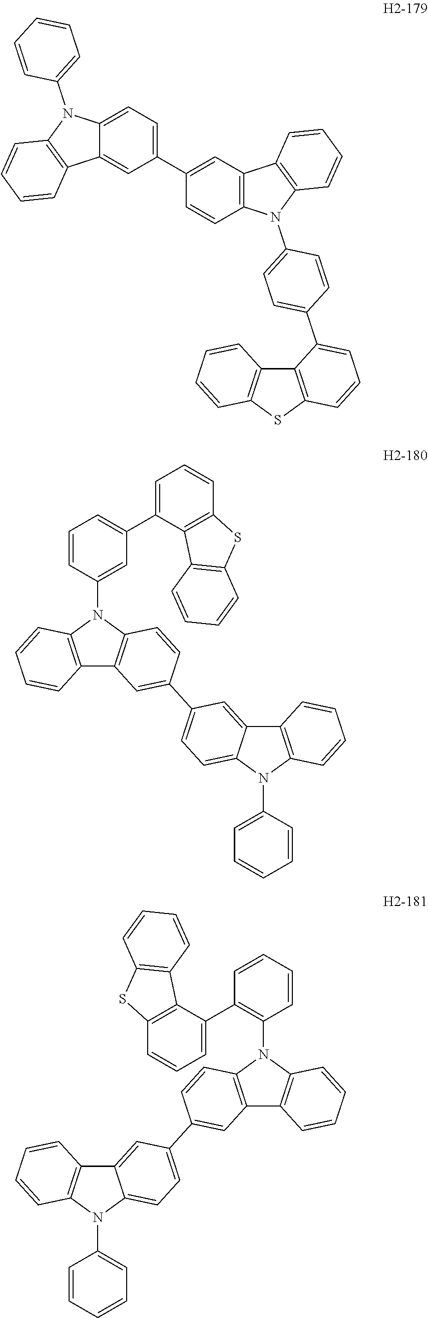 [SMILES: c1ccc(-n2c3ccccc3c3cc(-c4ccc5c(c4)c4ccccc4n5-c4ccc(-c5cccc6sc7ccccc7c56)cc4)ccc32)cc1 ; c1ccc(-n2c3ccccc3c3cc(-c4ccc5c(c4)c4ccccc4n5-c4cccc(-c5cccc6sc7ccccc7c56)c4)ccc32)cc1 ; c1ccc(-n2c3ccccc3c3cc(-c4ccc5c(c4)c4ccccc4n5-c4ccccc4-c4cccc5sc6ccccc6c45)ccc32)cc1]

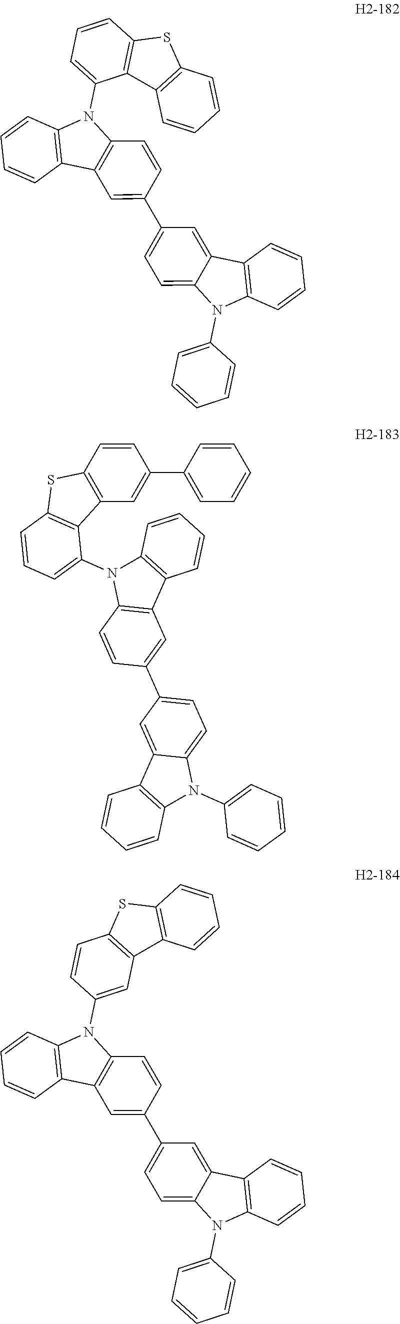

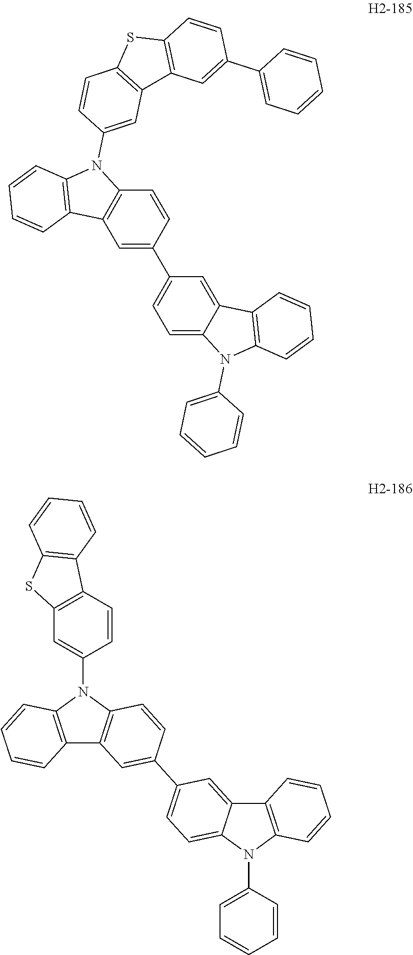 [SMILES: c1ccc(-c2ccc3sc4ccc(-n5c6ccccc6c6cc(-c7ccc8c(c7)c7ccccc7n8-c7ccccc7)ccc65)cc4c3c2)cc1 ; c1ccc(-n2c3ccccc3c3cc(-c4ccc5c(c4)c4ccccc4n5-c4ccc5c(c4)sc4ccccc45)ccc32)cc1]

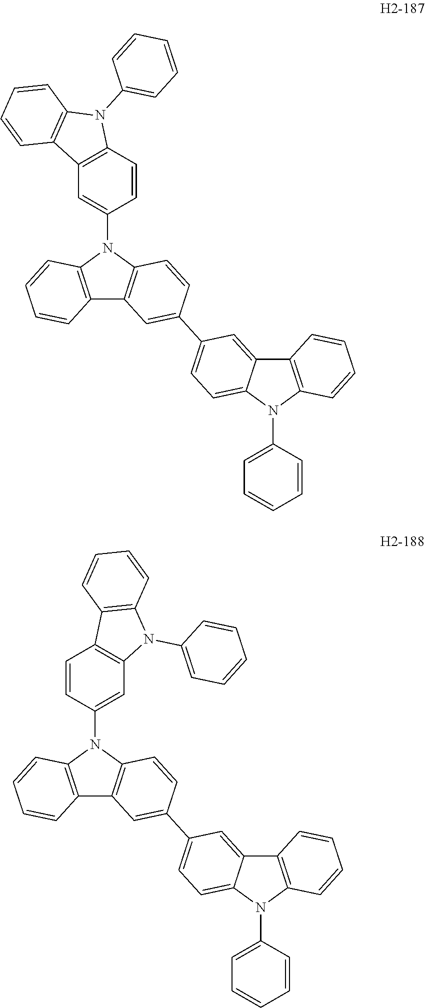 [SMILES: c1ccc(-n2c3ccccc3c3cc(-c4ccc5c(c4)c4ccccc4n5-c4ccc5c(c4)c4ccccc4n5-c4ccccc4)ccc32)cc1 ; c1ccc(-n2c3ccccc3c3cc(-c4ccc5c(c4)c4ccccc4n5-c4ccc5c6ccccc6n(-c6ccccc6)c5c4)ccc32)cc1]

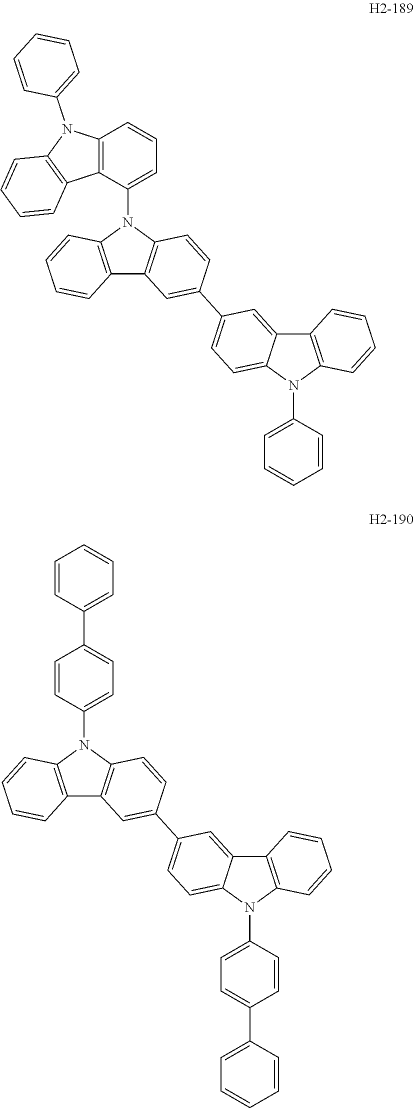 [SMILES: c1ccc(-n2c3ccccc3c3cc(-c4ccc5c(c4)c4ccccc4n5-c4cccc5c4c4ccccc4n5-c4ccccc4)ccc32)cc1 ; c1ccc(-c2ccc(-n3c4ccccc4c4cc(-c5ccc6c(c5)c5ccccc5n6-c5ccc(-c6ccccc6)cc5)ccc43)cc2)cc1]

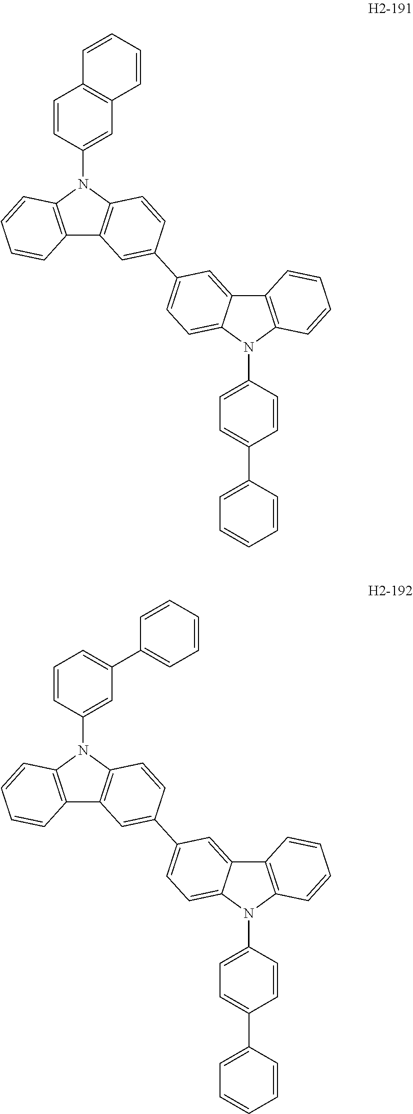 [SMILES: c1ccc(-c2ccc(-n3c4ccccc4c4cc(-c5ccc6c(c5)c5ccccc5n6-c5ccc6ccccc6c5)ccc43)cc2)cc1 ; c1ccc(-c2ccc(-n3c4ccccc4c4cc(-c5ccc6c(c5)c5ccccc5n6-c5cccc(-c6ccccc6)c5)ccc43)cc2)cc1]

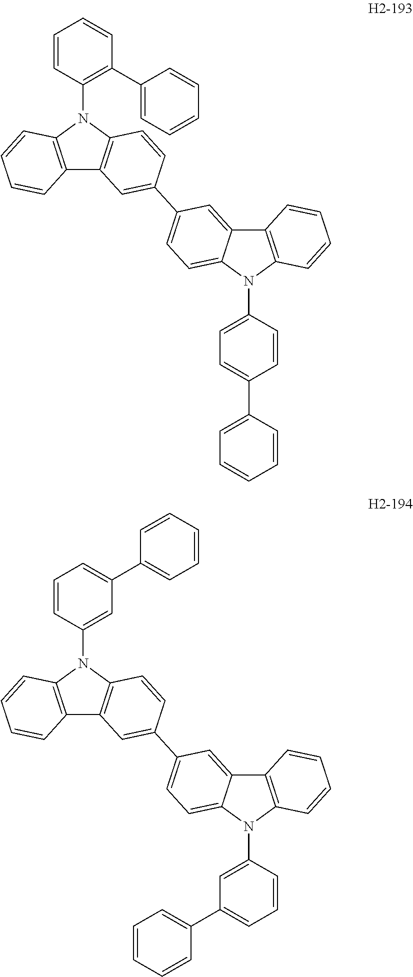 [SMILES: c1ccc(-c2ccc(-n3c4ccccc4c4cc(-c5ccc6c(c5)c5ccccc5n6-c5ccccc5-c5ccccc5)ccc43)cc2)cc1 ; c1ccc(-c2cccc(-n3c4ccccc4c4cc(-c5ccc6c(c5)c5ccccc5n6-c5cccc(-c6ccccc6)c5)ccc43)c2)cc1]

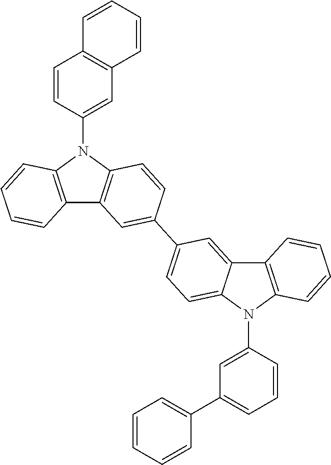 [SMILES: c1ccc(-c2cccc(-n3c4ccccc4c4cc(-c5ccc6c(c5)c5ccccc5n6-c5ccc6ccccc6c5)ccc43)c2)cc1]

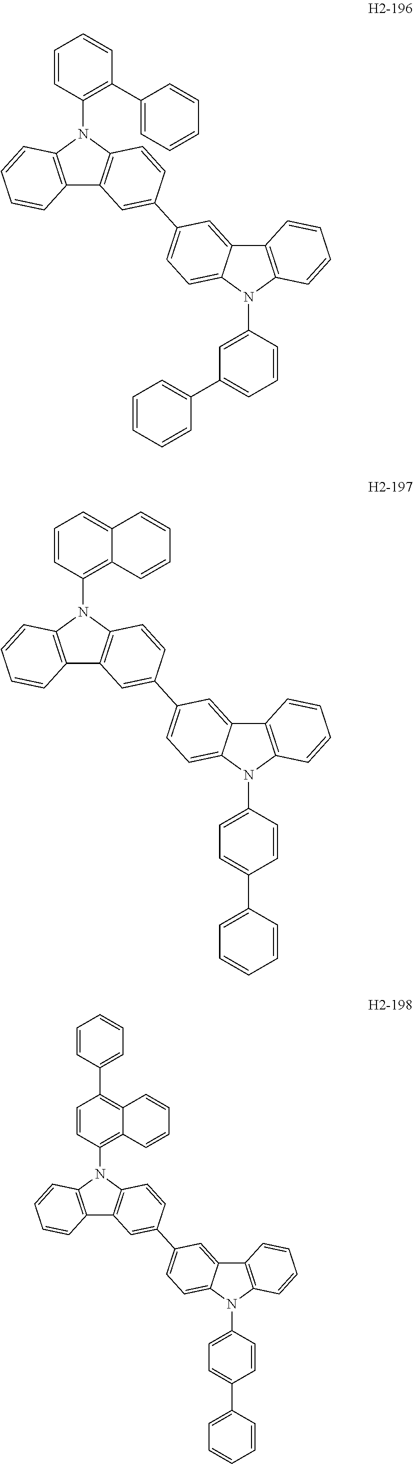 [SMILES: c1ccc(-c2cccc(-n3c4ccccc4c4cc(-c5ccc6c(c5)c5ccccc5n6-c5ccccc5-c5ccccc5)ccc43)c2)cc1 ; c1ccc(-c2ccc(-n3c4ccccc4c4cc(-c5ccc6c(c5)c5ccccc5n6-c5cccc6ccccc56)ccc43)cc2)cc1 ; c1ccc(-c2ccc(-n3c4ccccc4c4cc(-c5ccc6c(c5)c5ccccc5n6-c5ccc(-c6ccccc6)c6ccccc56)ccc43)cc2)cc1]

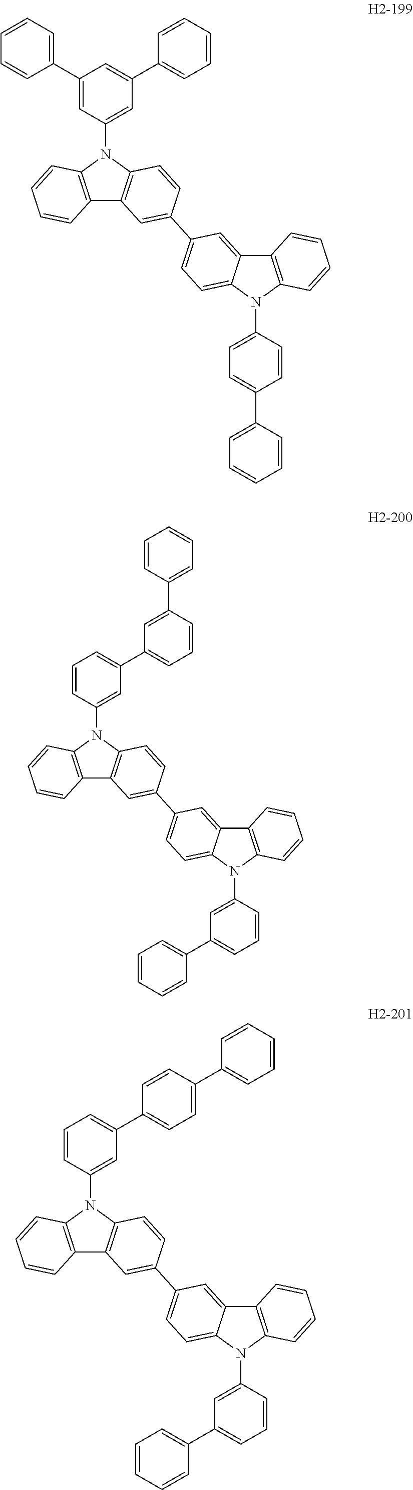 [SMILES: c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1 ; c1ccc(-c2cccc(-c3cccc(-n4c5ccccc5c5cc(-c6ccc7c(c6)c6ccccc6n7-c6cccc(-c7ccccc7)c6)ccc54)c3)c2)cc1 ; c1ccc(-c2ccc(-c3cccc(-n4c5ccccc5c5cc(-c6ccc7c(c6)c6ccccc6n7-c6cccc(-c7ccccc7)c6)ccc54)c3)cc2)cc1]

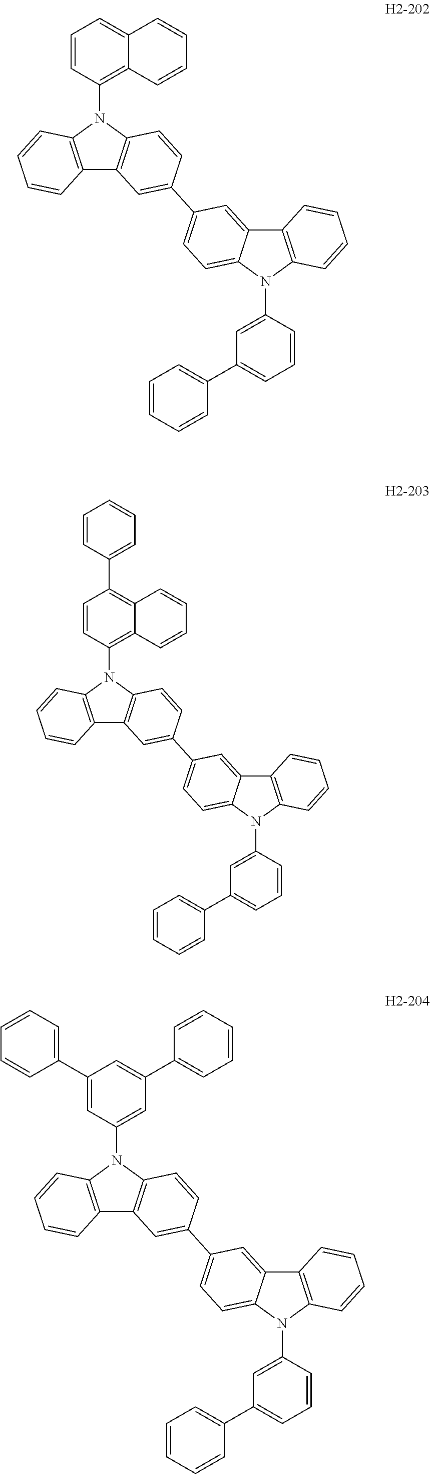 [SMILES: c1ccc(-c2cccc(-n3c4ccccc4c4cc(-c5ccc6c(c5)c5ccccc5n6-c5cccc6ccccc56)ccc43)c2)cc1 ; c1ccc(-c2cccc(-n3c4ccccc4c4cc(-c5ccc6c(c5)c5ccccc5n6-c5ccc(-c6ccccc6)c6ccccc56)ccc43)c2)cc1 ; c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1]

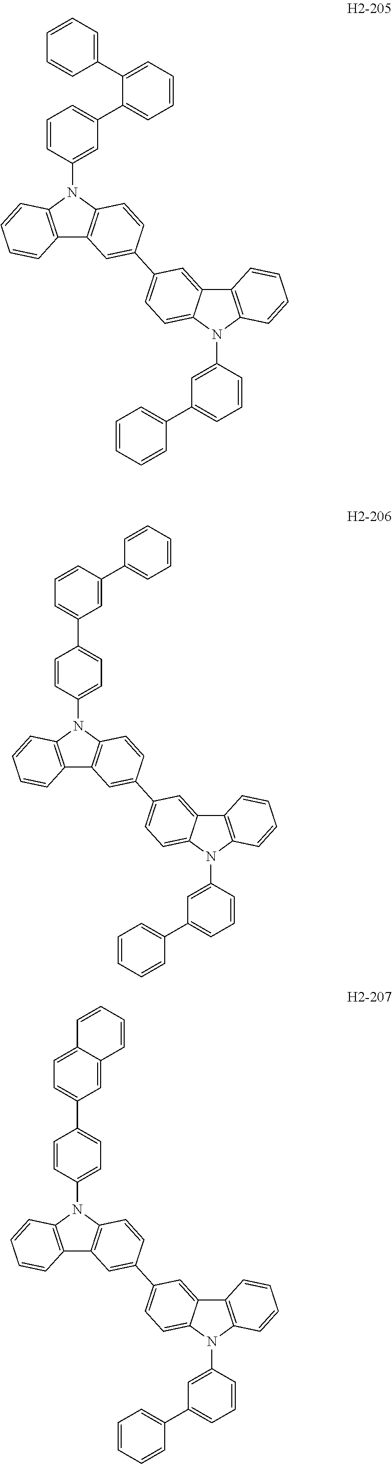 [SMILES: c1ccc(-c2cccc(-n3c4ccccc4c4cc(-c5ccc6c(c5)c5ccccc5n6-c5cccc(-c6ccccc6-c6ccccc6)c5)ccc43)c2)cc1 ; c1ccc(-c2cccc(-c3ccc(-n4c5ccccc5c5cc(-c6ccc7c(c6)c6ccccc6n7-c6cccc(-c7ccccc7)c6)ccc54)cc3)c2)cc1 ; c1ccc(-c2cccc(-n3c4ccccc4c4cc(-c5ccc6c(c5)c5ccccc5n6-c5ccc(-c6ccc7ccccc7c6)cc5)ccc43)c2)cc1]

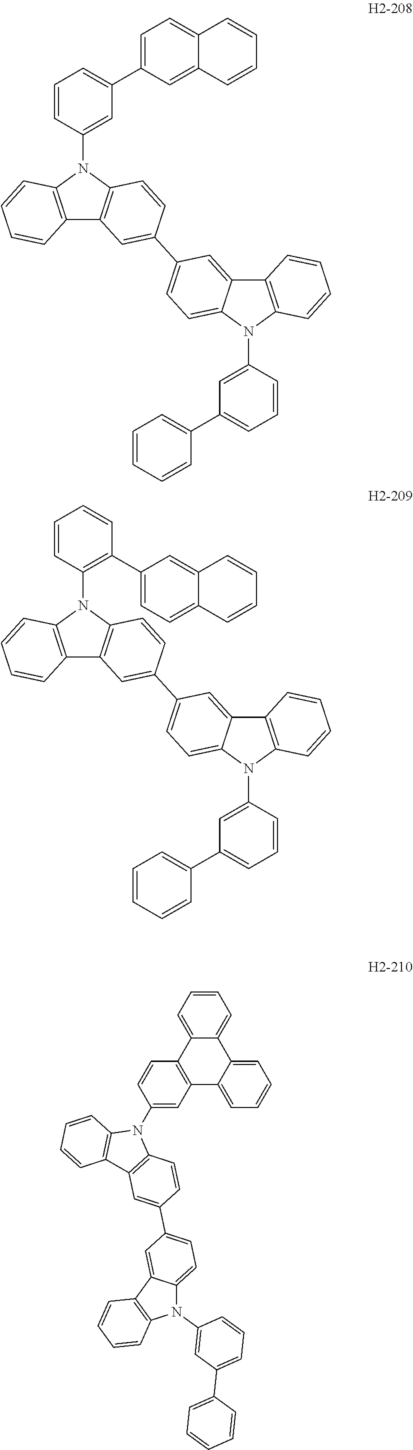 [SMILES: c1ccc(-c2cccc(-n3c4ccccc4c4cc(-c5ccc6c(c5)c5ccccc5n6-c5cccc(-c6ccc7ccccc7c6)c5)ccc43)c2)cc1 ; c1ccc(-c2cccc(-n3c4ccccc4c4cc(-c5ccc6c(c5)c5ccccc5n6-c5ccccc5-c5ccc6ccccc6c5)ccc43)c2)cc1 ; c1ccc(-c2cccc(-n3c4ccccc4c4cc(-c5ccc6c(c5)c5ccccc5n6-c5ccc6c7ccccc7c7ccccc7c6c5)ccc43)c2)cc1]

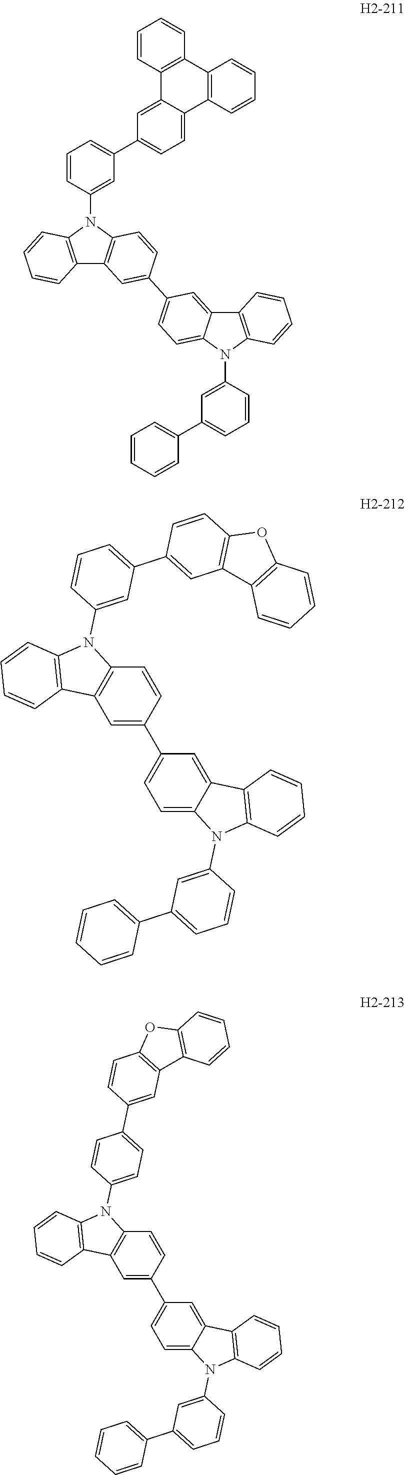 [SMILES: c1ccc(-c2cccc(-n3c4ccccc4c4cc(-c5ccc6c(c5)c5ccccc5n6-c5cccc(-c6ccc7c8ccccc8c8ccccc8c7c6)c5)ccc43)c2)cc1 ; c1ccc(-c2cccc(-n3c4ccccc4c4cc(-c5ccc6c(c5)c5ccccc5n6-c5cccc(-c6ccc7oc8ccccc8c7c6)c5)ccc43)c2)cc1 ; c1ccc(-c2cccc(-n3c4ccccc4c4cc(-c5ccc6c(c5)c5ccccc5n6-c5ccc(-c6ccc7oc8ccccc8c7c6)cc5)ccc43)c2)cc1]

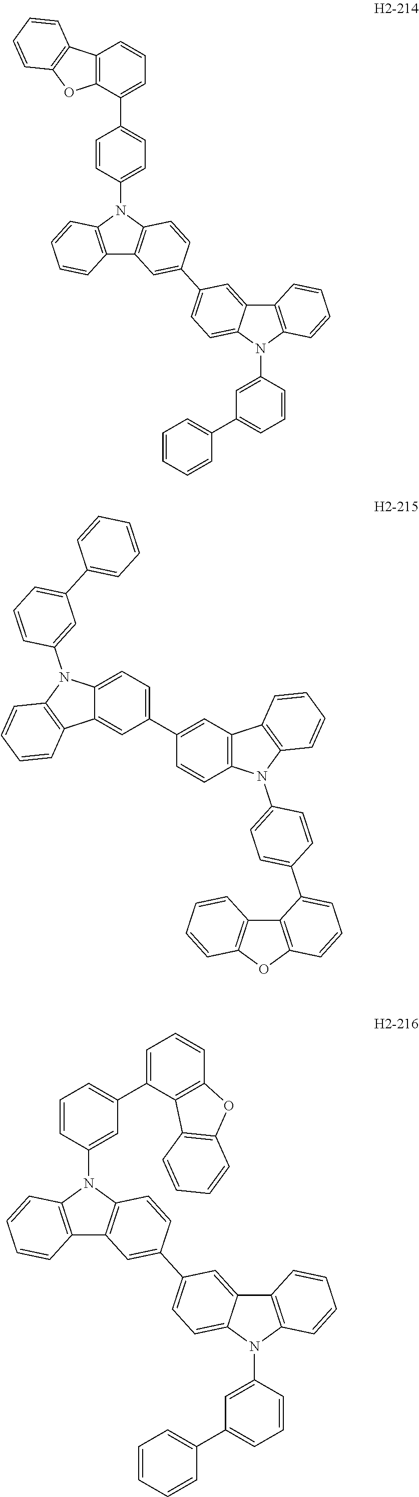 [SMILES: c1ccc(-c2cccc(-n3c4ccccc4c4cc(-c5ccc6c(c5)c5ccccc5n6-c5ccc(-c6cccc7c6oc6ccccc67)cc5)ccc43)c2)cc1 ; c1ccc(-c2cccc(-n3c4ccccc4c4cc(-c5ccc6c(c5)c5ccccc5n6-c5ccc(-c6cccc7oc8ccccc8c67)cc5)ccc43)c2)cc1 ; c1ccc(-c2cccc(-n3c4ccccc4c4cc(-c5ccc6c(c5)c5ccccc5n6-c5cccc(-c6cccc7oc8ccccc8c67)c5)ccc43)c2)cc1]

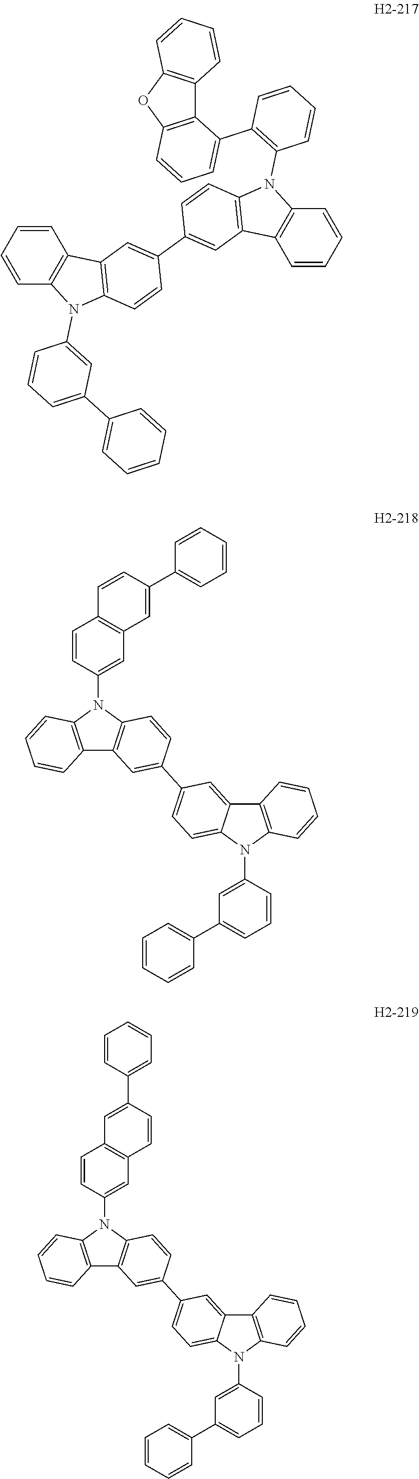 [SMILES: c1ccc(-c2cccc(-n3c4ccccc4c4cc(-c5ccc6c(c5)c5ccccc5n6-c5ccccc5-c5cccc6oc7ccccc7c56)ccc43)c2)cc1 ; c1ccc(-c2cccc(-n3c4ccccc4c4cc(-c5ccc6c(c5)c5ccccc5n6-c5ccc6ccc(-c7ccccc7)cc6c5)ccc43)c2)cc1 ; c1ccc(-c2cccc(-n3c4ccccc4c4cc(-c5ccc6c(c5)c5ccccc5n6-c5ccc6cc(-c7ccccc7)ccc6c5)ccc43)c2)cc1]

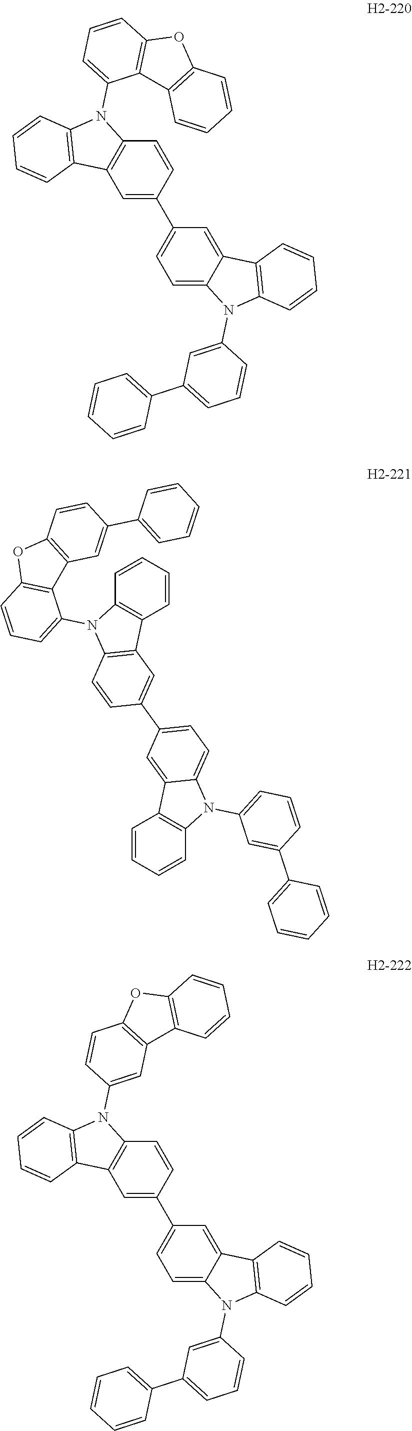 [SMILES: c1ccc(-c2cccc(-n3c4ccccc4c4cc(-c5ccc6c(c5)c5ccccc5n6-c5cccc6oc7ccccc7c56)ccc43)c2)cc1 ; c1ccc(-c2cccc(-n3c4ccccc4c4cc(-c5ccc6c(c5)c5ccccc5n6-c5cccc6oc7ccc(-c8ccccc8)cc7c56)ccc43)c2)cc1 ; c1ccc(-c2cccc(-n3c4ccccc4c4cc(-c5ccc6c(c5)c5ccccc5n6-c5ccc6oc7ccccc7c6c5)ccc43)c2)cc1]

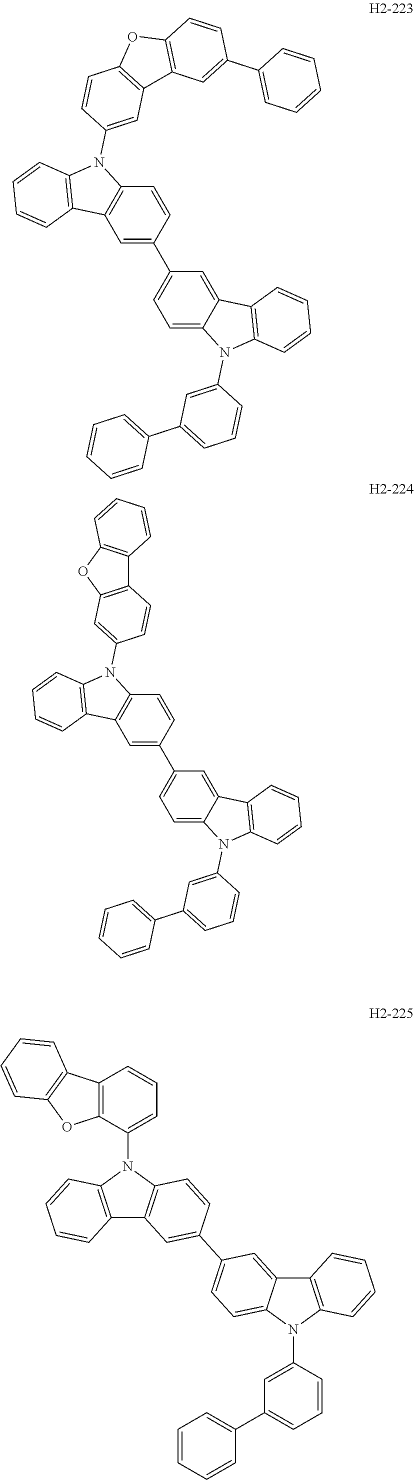

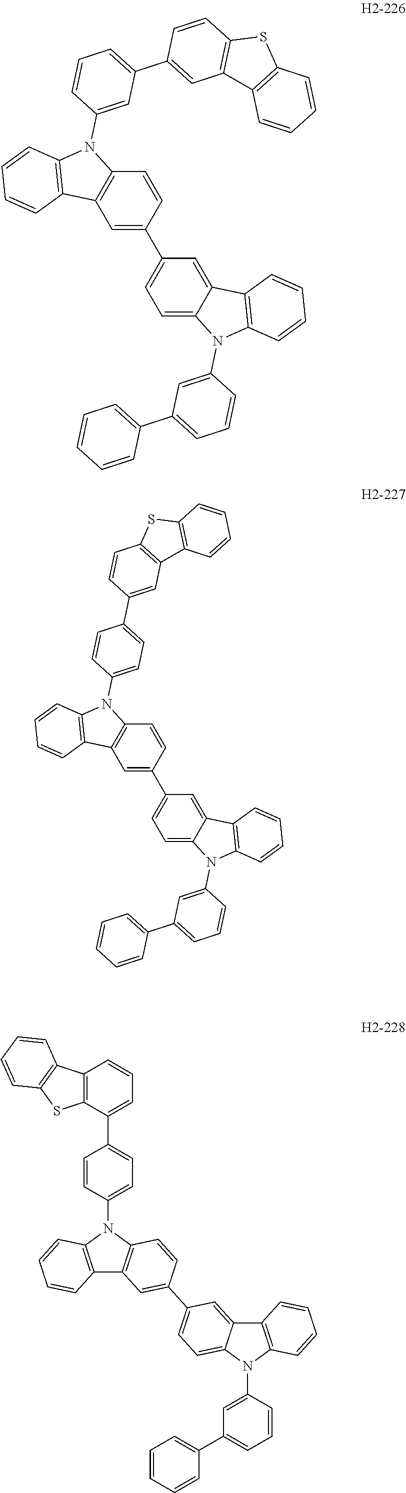 [SMILES: c1ccc(-c2cccc(-n3c4ccccc4c4cc(-c5ccc6c(c5)c5ccccc5n6-c5cccc(-c6ccc7sc8ccccc8c7c6)c5)ccc43)c2)cc1 ; c1ccc(-c2cccc(-n3c4ccccc4c4cc(-c5ccc6c(c5)c5ccccc5n6-c5ccc(-c6ccc7sc8ccccc8c7c6)cc5)ccc43)c2)cc1 ; c1ccc(-c2cccc(-n3c4ccccc4c4cc(-c5ccc6c(c5)c5ccccc5n6-c5ccc(-c6cccc7c6sc6ccccc67)cc5)ccc43)c2)cc1]

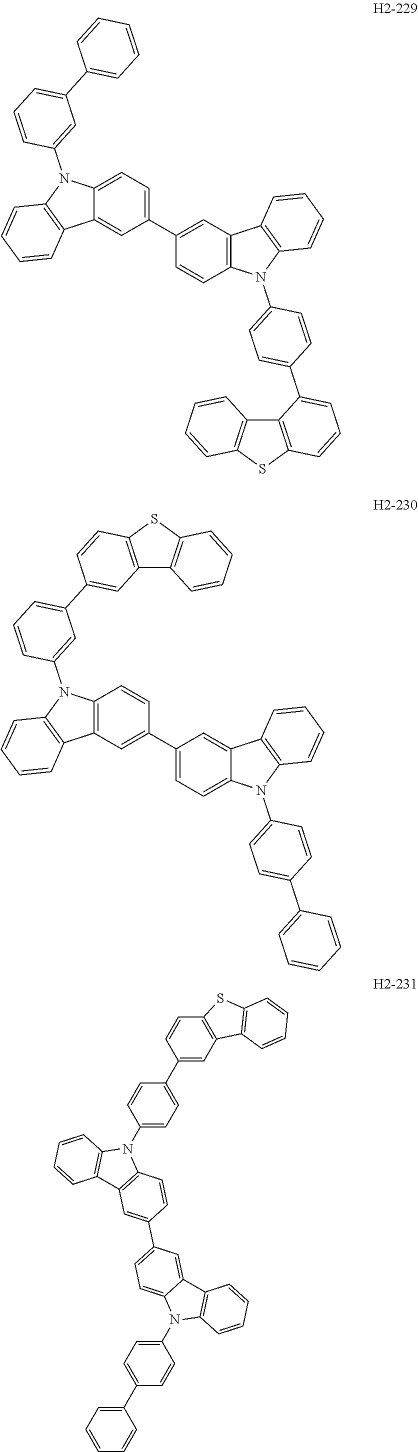

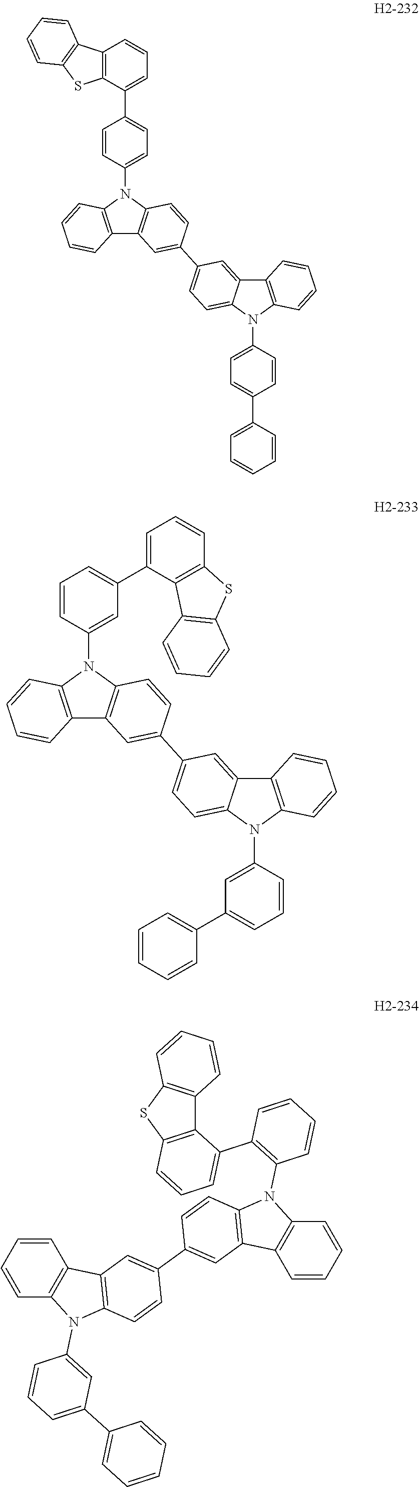 [SMILES: c1ccc(-c2ccc(-n3c4ccccc4c4cc(-c5ccc6c(c5)c5ccccc5n6-c5ccc(-c6cccc7c6sc6ccccc67)cc5)ccc43)cc2)cc1 ; c1ccc(-c2cccc(-n3c4ccccc4c4cc(-c5ccc6c(c5)c5ccccc5n6-c5cccc(-c6cccc7sc8ccccc8c67)c5)ccc43)c2)cc1 ; c1ccc(-c2cccc(-n3c4ccccc4c4cc(-c5ccc6c(c5)c5ccccc5n6-c5ccccc5-c5cccc6sc7ccccc7c56)ccc43)c2)cc1]

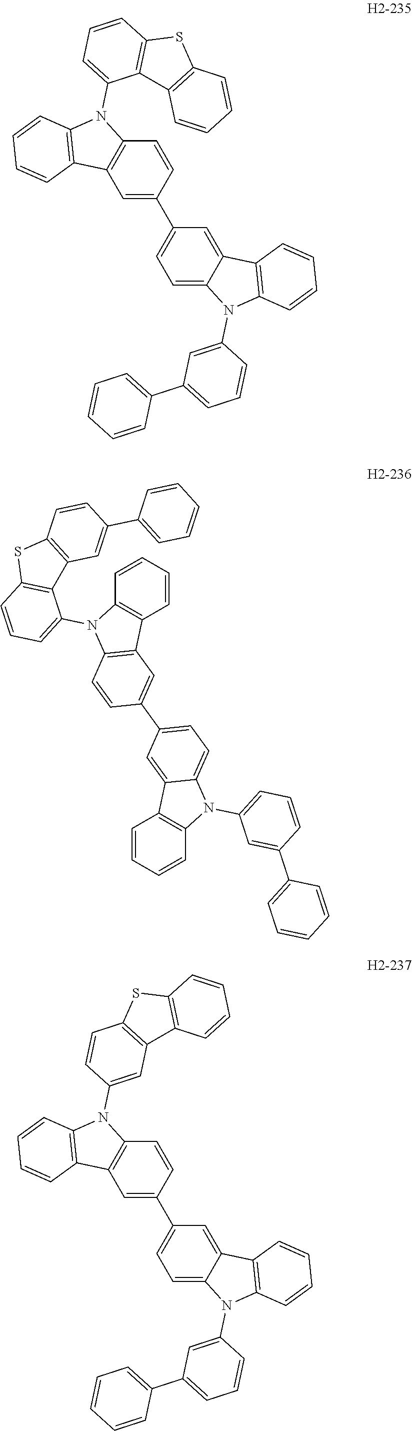 [SMILES: c1ccc(-c2cccc(-n3c4ccccc4c4cc(-c5ccc6c(c5)c5ccccc5n6-c5cccc6sc7ccccc7c56)ccc43)c2)cc1 ; c1ccc(-c2cccc(-n3c4ccccc4c4cc(-c5ccc6c(c5)c5ccccc5n6-c5cccc6sc7ccc(-c8ccccc8)cc7c56)ccc43)c2)cc1 ; c1ccc(-c2cccc(-n3c4ccccc4c4cc(-c5ccc6c(c5)c5ccccc5n6-c5ccc6sc7ccccc7c6c5)ccc43)c2)cc1]

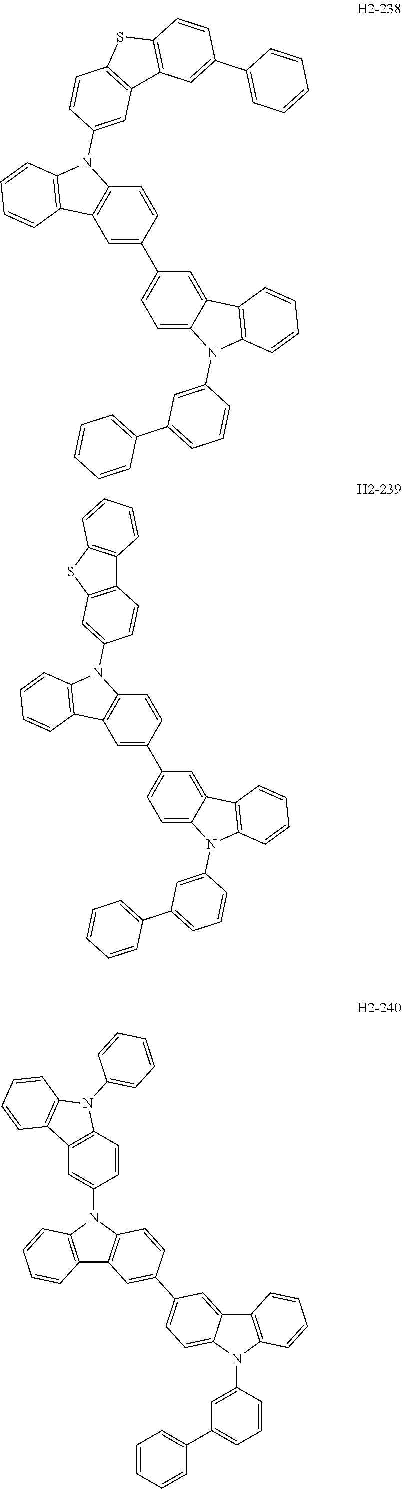

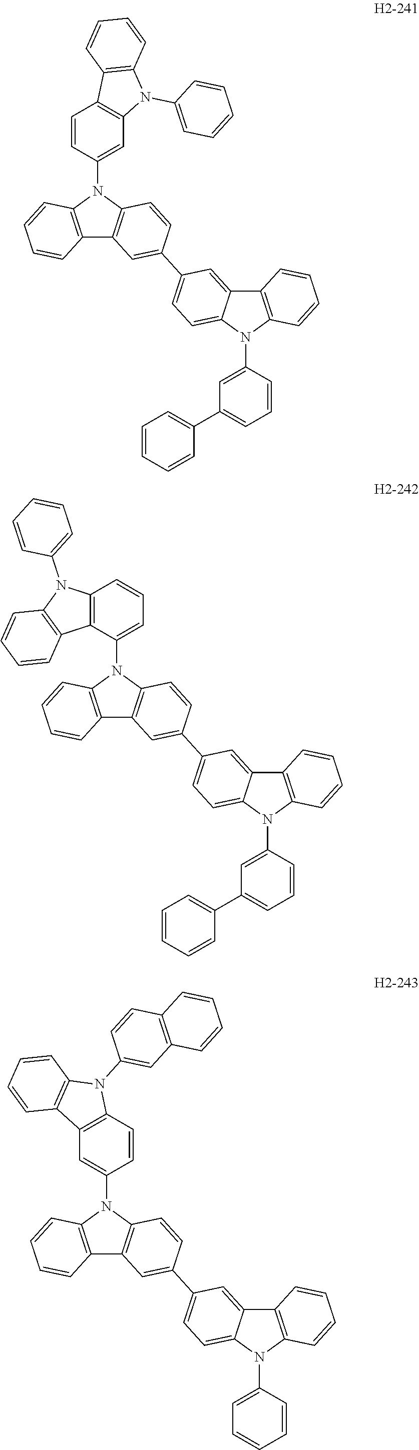

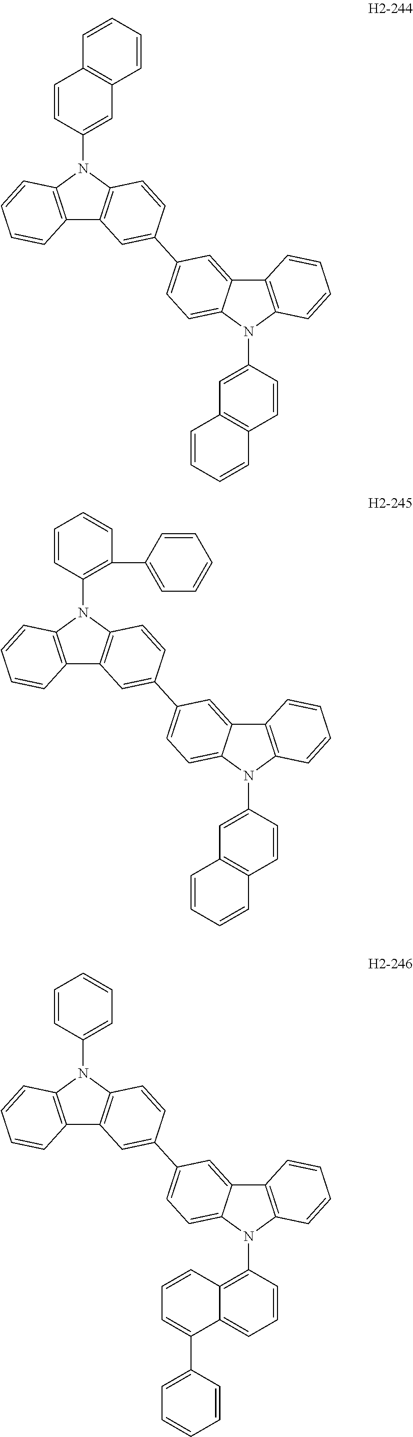 [SMILES: c1ccc2cc(-n3c4ccccc4c4cc(-c5ccc6c(c5)c5ccccc5n6-c5ccc6ccccc6c5)ccc43)ccc2c1 ; c1ccc(-c2ccccc2-n2c3ccccc3c3cc(-c4ccc5c(c4)c4ccccc4n5-c4ccc5ccccc5c4)ccc32)cc1 ; c1ccc(-c2cccc3c(-n4c5ccccc5c5cc(-c6ccc7c(c6)c6ccccc6n7-c6ccccc6)ccc54)cccc23)cc1]

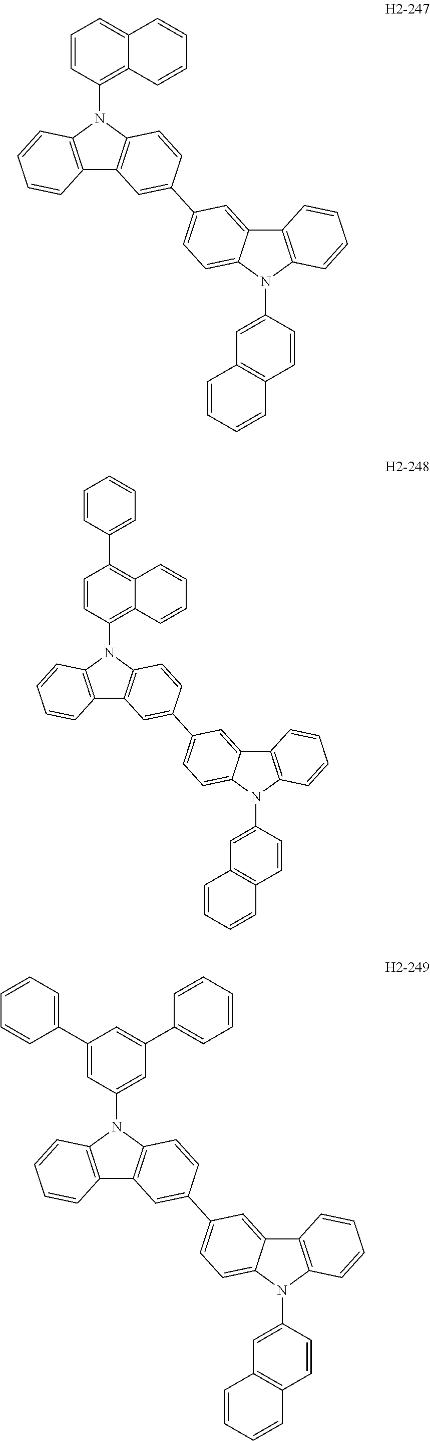 [SMILES: c1ccc2cc(-n3c4ccccc4c4cc(-c5ccc6c(c5)c5ccccc5n6-c5cccc6ccccc56)ccc43)ccc2c1 ; c1ccc(-c2ccc(-n3c4ccccc4c4cc(-c5ccc6c(c5)c5ccccc5n6-c5ccc6ccccc6c5)ccc43)c3ccccc23)cc1 ; c1ccc(-c2cc(-c3ccccc3)cc(-n3c4ccccc4c4cc(-c5ccc6c(c5)c5ccccc5n6-c5ccc6ccccc6c5)ccc43)c2)cc1]

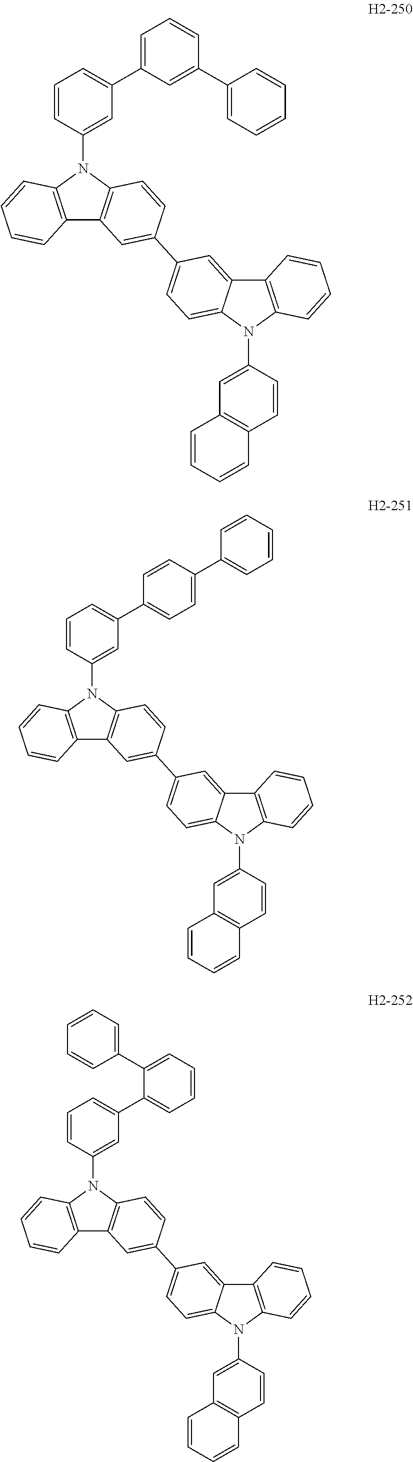 [SMILES: c1ccc(-c2cccc(-c3cccc(-n4c5ccccc5c5cc(-c6ccc7c(c6)c6ccccc6n7-c6ccc7ccccc7c6)ccc54)c3)c2)cc1 ; c1ccc(-c2ccc(-c3cccc(-n4c5ccccc5c5cc(-c6ccc7c(c6)c6ccccc6n7-c6ccc7ccccc7c6)ccc54)c3)cc2)cc1 ; c1ccc(-c2ccccc2-c2cccc(-n3c4ccccc4c4cc(-c5ccc6c(c5)c5ccccc5n6-c5ccc6ccccc6c5)ccc43)c2)cc1]

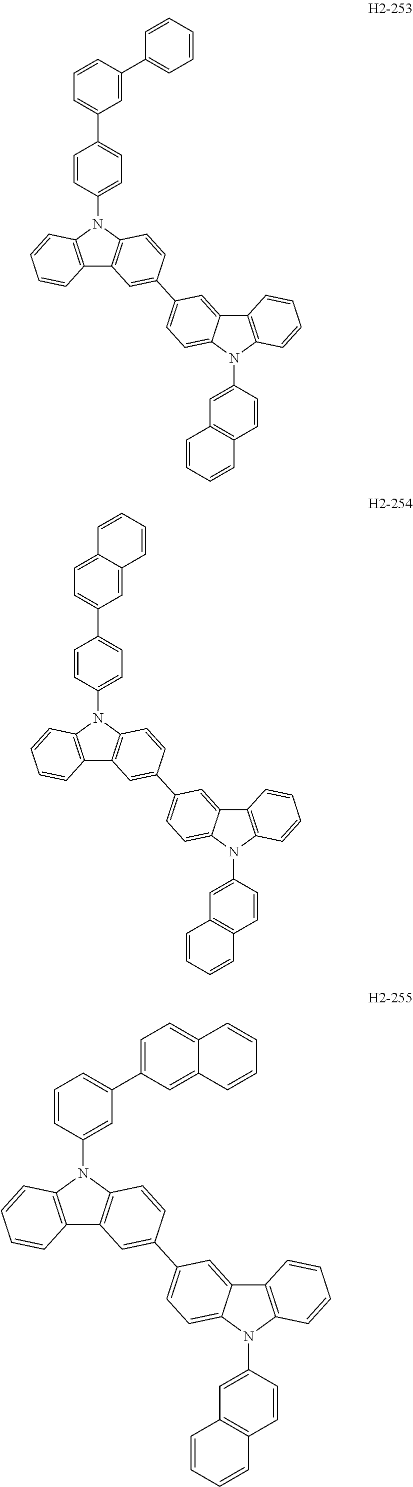 [SMILES: c1ccc(-c2cccc(-c3ccc(-n4c5ccccc5c5cc(-c6ccc7c(c6)c6ccccc6n7-c6ccc7ccccc7c6)ccc54)cc3)c2)cc1 ; c1ccc2cc(-c3ccc(-n4c5ccccc5c5cc(-c6ccc7c(c6)c6ccccc6n7-c6ccc7ccccc7c6)ccc54)cc3)ccc2c1 ; c1cc(-c2ccc3ccccc3c2)cc(-n2c3ccccc3c3cc(-c4ccc5c(c4)c4ccccc4n5-c4ccc5ccccc5c4)ccc32)c1]

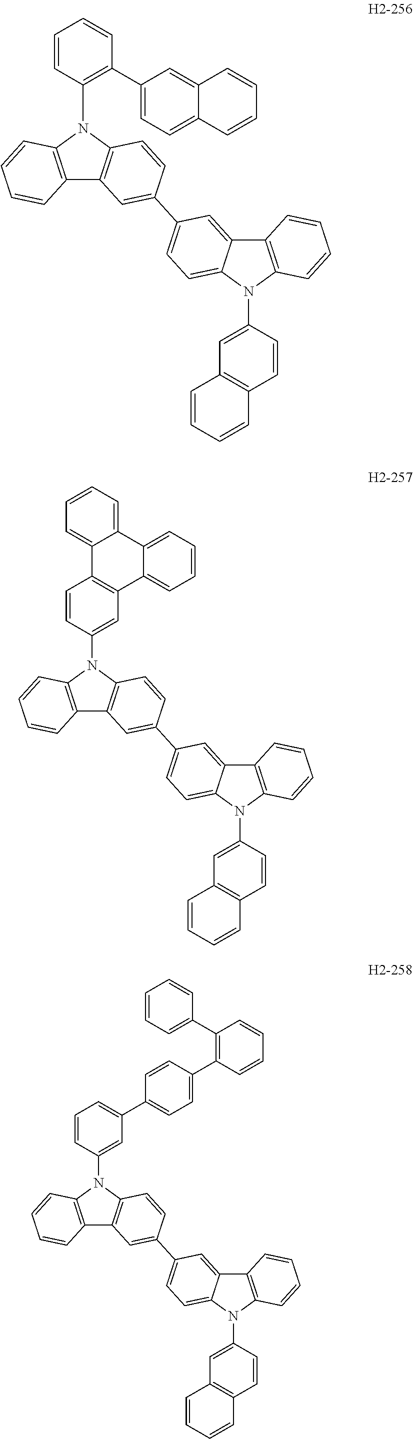 [SMILES: c1ccc(-n2c3ccccc3c3cc(-c4ccc5c(c4)c4ccccc4n5-c4ccc5ccccc5c4)ccc32)c(-c2ccc3ccccc3c2)c1 ; c1ccc2cc(-n3c4ccccc4c4cc(-c5ccc6c(c5)c5ccccc5n6-c5ccc6c7ccccc7c7ccccc7c6c5)ccc43)ccc2c1 ; c1ccc(-c2ccccc2-c2ccc(-c3cccc(-n4c5ccccc5c5cc(-c6ccc7c(c6)c6ccccc6n7-c6ccc7ccccc7c6)ccc54)c3)cc2)cc1]

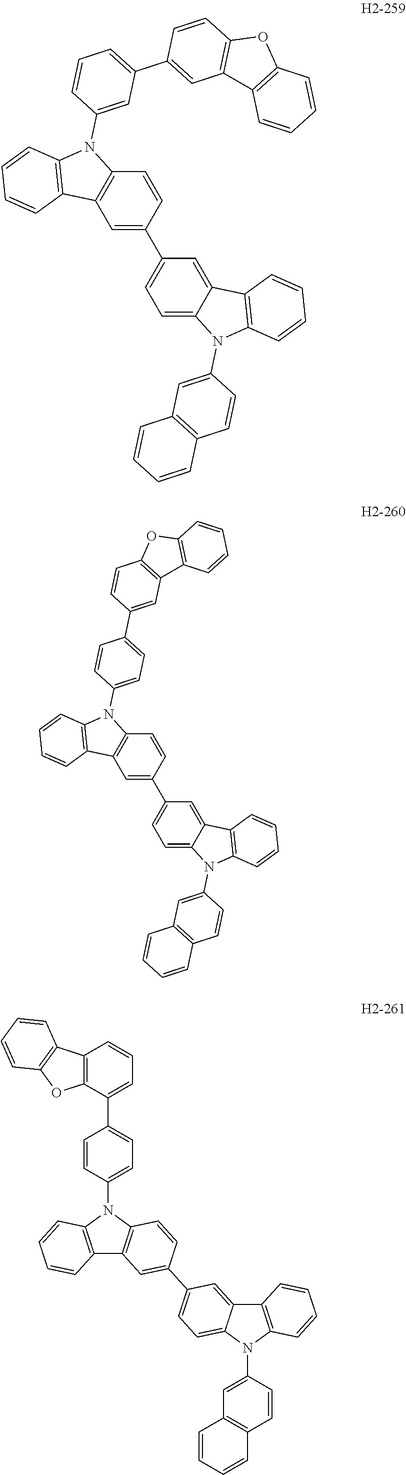 [SMILES: c1cc(-c2ccc3oc4ccccc4c3c2)cc(-n2c3ccccc3c3cc(-c4ccc5c(c4)c4ccccc4n5-c4ccc5ccccc5c4)ccc32)c1 ; c1ccc2cc(-n3c4ccccc4c4cc(-c5ccc6c(c5)c5ccccc5n6-c5ccc(-c6ccc7oc8ccccc8c7c6)cc5)ccc43)ccc2c1 ; c1ccc2cc(-n3c4ccccc4c4cc(-c5ccc6c(c5)c5ccccc5n6-c5ccc(-c6cccc7c6oc6ccccc67)cc5)ccc43)ccc2c1]

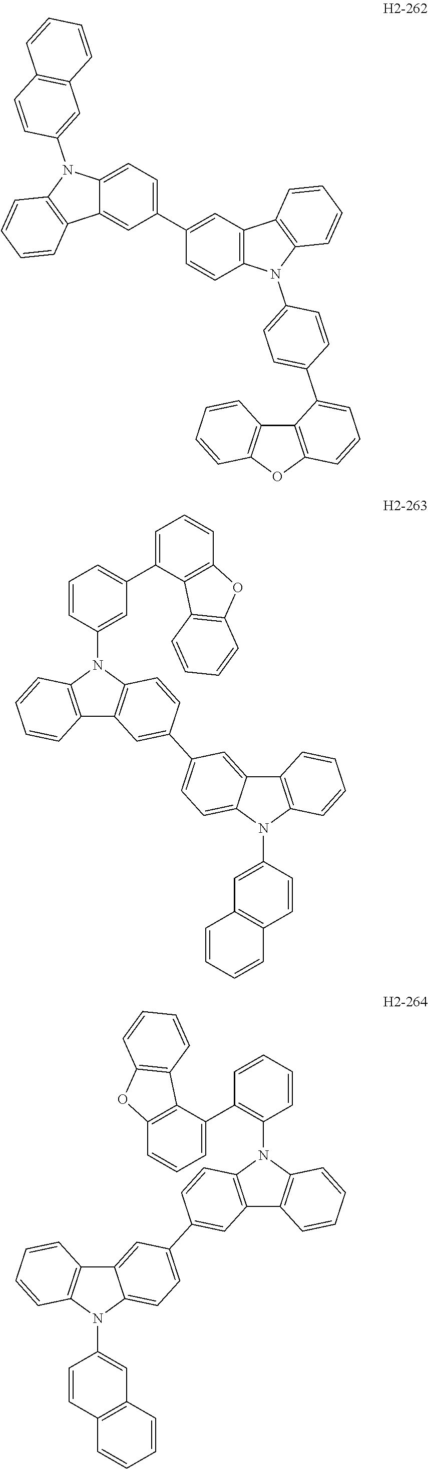 [SMILES: c1ccc2cc(-n3c4ccccc4c4cc(-c5ccc6c(c5)c5ccccc5n6-c5ccc(-c6cccc7oc8ccccc8c67)cc5)ccc43)ccc2c1 ; c1cc(-c2cccc3oc4ccccc4c23)cc(-n2c3ccccc3c3cc(-c4ccc5c(c4)c4ccccc4n5-c4ccc5ccccc5c4)ccc32)c1 ; c1ccc(-n2c3ccccc3c3cc(-c4ccc5c(c4)c4ccccc4n5-c4ccc5ccccc5c4)ccc32)c(-c2cccc3oc4ccccc4c23)c1]

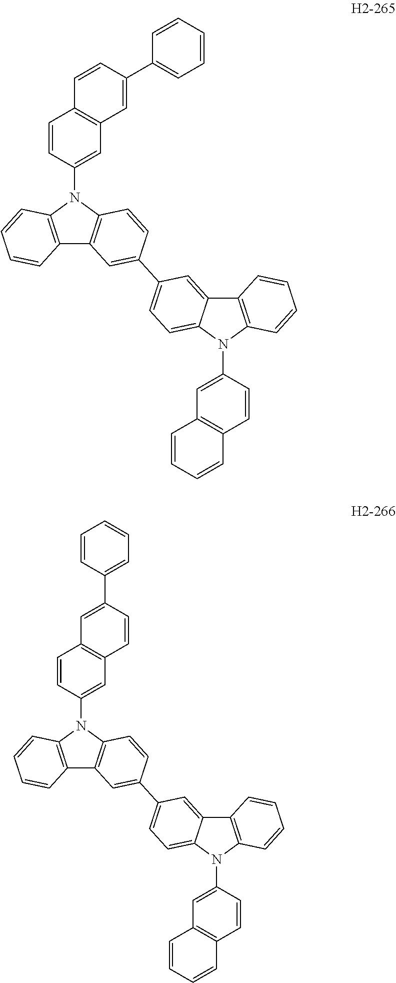 [SMILES: c1ccc(-c2ccc3ccc(-n4c5ccccc5c5cc(-c6ccc7c(c6)c6ccccc6n7-c6ccc7ccccc7c6)ccc54)cc3c2)cc1 ; c1ccc(-c2ccc3cc(-n4c5ccccc5c5cc(-c6ccc7c(c6)c6ccccc6n7-c6ccc7ccccc7c6)ccc54)ccc3c2)cc1]

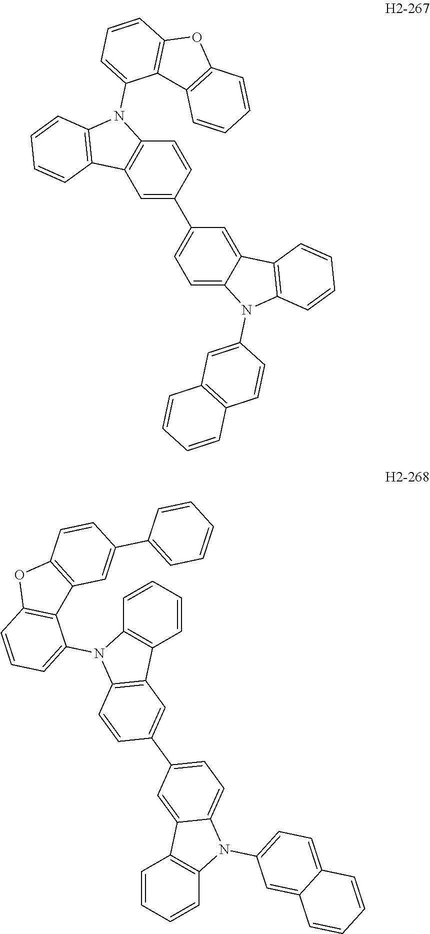 [SMILES: c1ccc2cc(-n3c4ccccc4c4cc(-c5ccc6c(c5)c5ccccc5n6-c5cccc6oc7ccccc7c56)ccc43)ccc2c1 ; c1ccc(-c2ccc3oc4cccc(-n5c6ccccc6c6cc(-c7ccc8c(c7)c7ccccc7n8-c7ccc8ccccc8c7)ccc65)c4c3c2)cc1]

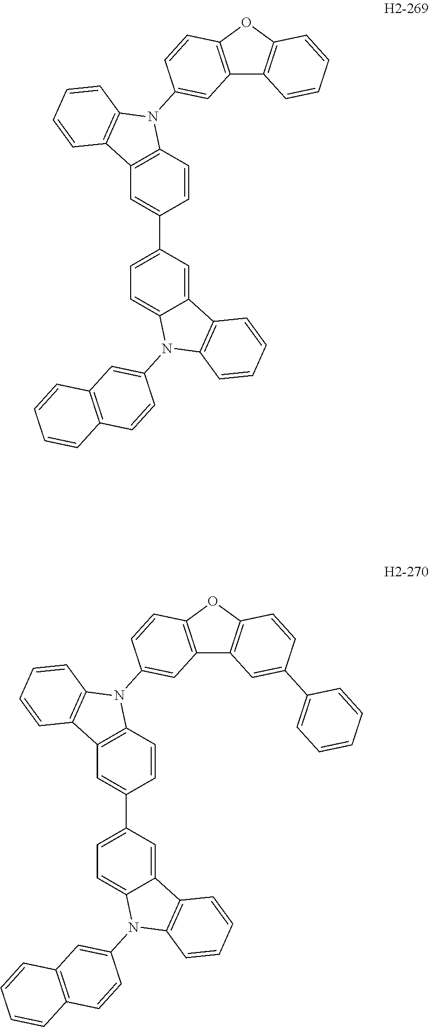 [SMILES: c1ccc2cc(-n3c4ccccc4c4cc(-c5ccc6c(c5)c5ccccc5n6-c5ccc6oc7ccccc7c6c5)ccc43)ccc2c1 ; c1ccc(-c2ccc3oc4ccc(-n5c6ccccc6c6cc(-c7ccc8c(c7)c7ccccc7n8-c7ccc8ccccc8c7)ccc65)cc4c3c2)cc1]

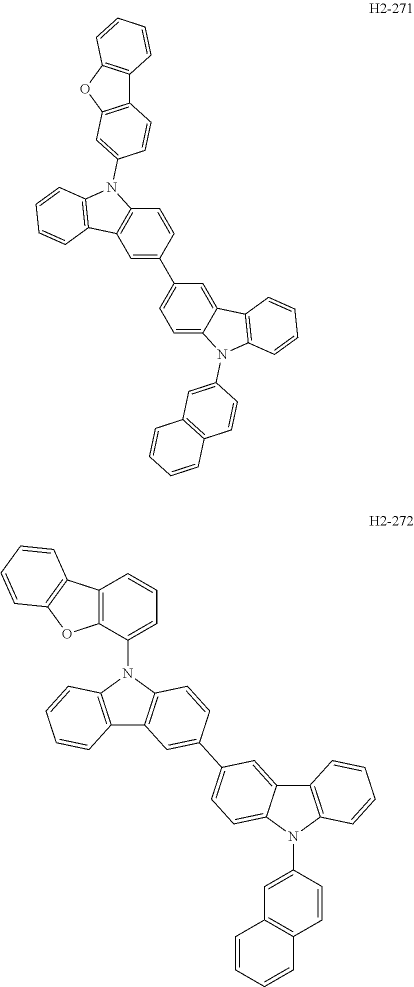 [SMILES: c1ccc2cc(-n3c4ccccc4c4cc(-c5ccc6c(c5)c5ccccc5n6-c5ccc6c(c5)oc5ccccc56)ccc43)ccc2c1 ; c1ccc2cc(-n3c4ccccc4c4cc(-c5ccc6c(c5)c5ccccc5n6-c5cccc6c5oc5ccccc56)ccc43)ccc2c1]

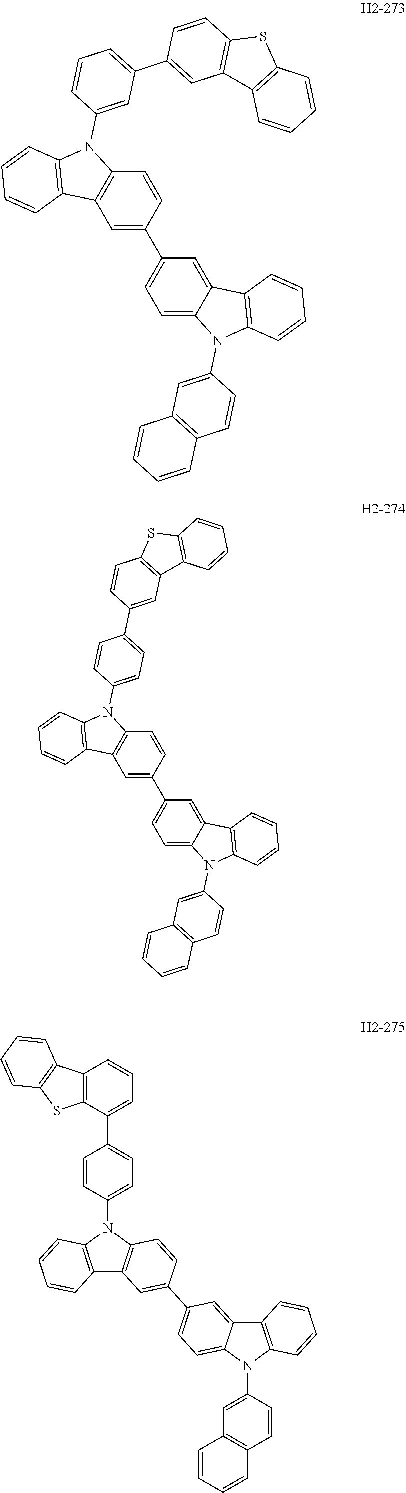 [SMILES: c1cc(-c2ccc3sc4ccccc4c3c2)cc(-n2c3ccccc3c3cc(-c4ccc5c(c4)c4ccccc4n5-c4ccc5ccccc5c4)ccc32)c1 ; c1ccc2cc(-n3c4ccccc4c4cc(-c5ccc6c(c5)c5ccccc5n6-c5ccc(-c6ccc7sc8ccccc8c7c6)cc5)ccc43)ccc2c1 ; c1ccc2cc(-n3c4ccccc4c4cc(-c5ccc6c(c5)c5ccccc5n6-c5ccc(-c6cccc7c6sc6ccccc67)cc5)ccc43)ccc2c1]

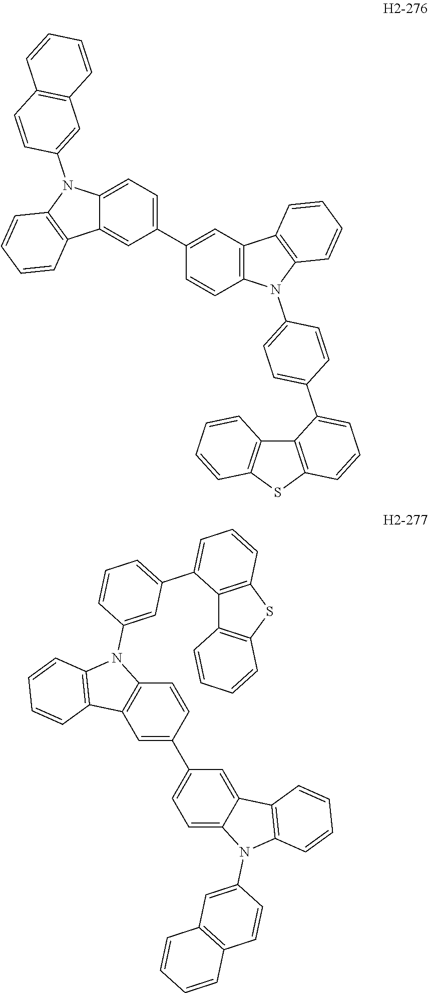 [SMILES: c1ccc2cc(-n3c4ccccc4c4cc(-c5ccc6c(c5)c5ccccc5n6-c5ccc(-c6cccc7sc8ccccc8c67)cc5)ccc43)ccc2c1 ; c1cc(-c2cccc3sc4ccccc4c23)cc(-n2c3ccccc3c3cc(-c4ccc5c(c4)c4ccccc4n5-c4ccc5ccccc5c4)ccc32)c1]

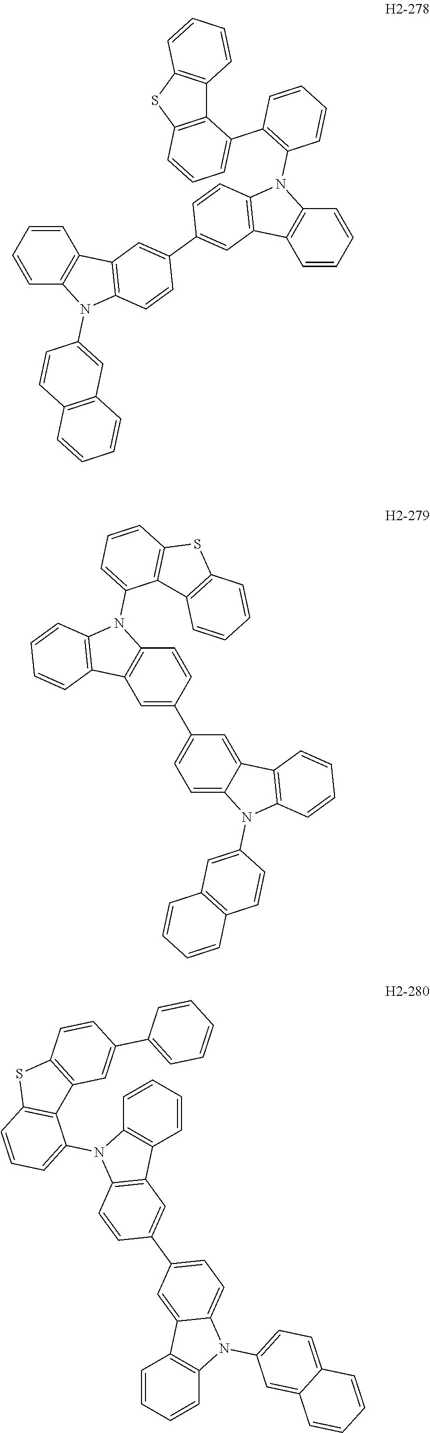

In the compounds H2-1 to H2-144, Dₙ means that n, the number of hydrogens are substituted with deuterium. In formula 2, the deuterium substitution rate may be about 40% to about 100%, preferably about 50% to about 100%, more preferably about 60% to about 100%, and still more preferably about 70% to about 100%. When deuterated to a number equal to or greater than the lower limit, bond dissociation energy due to deuteration increases to increase the stability of the compound, and when the compound is used in an organic electroluminescent device, the device may exhibit improved lifespan characteristics.

A combination of at least one of compounds C-1 to C-255 and at least one of compounds H2-1 to H2-280 may be used in an organic electroluminescent device.

Hereinafter, the compound represented by formula 3 will be described in more detail.

In formula 3, X represents O or S.

In formula 3, R′₁ represents hydrogen, deuterium, or a phenyl unsubstituted or substituted with deuterium. According to one embodiment of the present disclosure, R′₁ represents a phenyl unsubstituted or substituted with deuterium.

In formula 3, R′₂ to R′₅ each independently represent hydrogen, deuterium, a substituted or unsubstituted (C6-C30)aryl, or formula a′; with a proviso that at least one of R′₂ to R′₅ represents formula a′. According to one embodiment of the present disclosure, R′₂ to R′₅ each independently represent hydrogen, deuterium, or formula a′. According to another embodiment, any one of R′₂ to R′₅ each independently represents formula a′, and the others each independently represent hydrogen or deuterium.

L′₁ represents a single bond, or a substituted or unsubstituted (C6-C30)arylene. According to one embodiment of the present disclosure, L′₁ represents a single bond, or a (C6-C30)arylene unsubstituted or substituted with at least one of deuterium and a (C6-C30)aryl(s). According to another embodiment of the present disclosure, L′₁ represents a single bond, or a (C6-C20)arylene unsubstituted or substituted with at least one of deuterium and a (C6-C20)aryl(s). For example, L′₁ may represent a single bond, a phenylene unsubstituted or substituted with a phenyl(s), a biphenylene unsubstituted or substituted with a phenyl(s), or a terphenylene, which may be further substituted with deuterium.

Y′₁ to Y′₃ each independently represent N or CH; with a proviso that at least two of Y′₁ to Y′₃ represent N. For example, two of Y′₁ to Y′₃ are N and the other is CH; or Y′₁ to Y′₃ are all N.

Ar′₁ and Ar′₂ each independently represent a substituted or unsubstituted (C6-C30)aryl, a substituted or unsubstituted carbazolyl, a substituted or unsubstituted dibenzofuranyl, or a substituted or unsubstituted dibenzothiophenyl; with a proviso that at least one of Ar′₁ and Ar′₂ represents a substituted or unsubstituted carbazolyl, a substituted or unsubstituted dibenzofuranyl, or a substituted or unsubstituted dibenzothiophenyl, and Ar′₁ and Ar′₂ are different from each other. According to one embodiment of the present disclosure, when Ar′₁ and Ar′₂ represent each independently a substituted or unsubstituted carbazolyl, a substituted or unsubstituted dibenzofuranyl, or a substituted or unsubstituted dibenzothiophenyl, they may be each differently selected from the group consisting of a substituted or unsubstituted carbazolyl, a substituted or unsubstituted dibenzofuranyl, and a substituted or unsubstituted dibenzothiophenyl. According to another embodiment of the present disclosure, Ar′₁ and Ar′₂ each independently represent a (C6-C18)aryl unsubstituted or substituted with deuterium; a carbazolyl unsubstituted or substituted with at least one of deuterium and a (C6-C20)aryl(s); a dibenzofuranyl unsubstituted or substituted with at least one of deuterium and a (C6-C18)aryl(s); or a dibenzothiophenyl unsubstituted or substituted with at least one of deuterium and a (C6-C18)aryl(s). For example, Ar′₁ and Ar′₂, each independently, may represent a phenyl, a biphenyl, a terphenyl, a dibenzofuranyl unsubstituted or substituted with a phenyl(s), a dibenzothiophenyl unsubstituted or substituted with a phenyl(s), or a carbazolyl unsubstituted or substituted with at least one of a phenyl(s) and a biphenyl(s), which may be further substituted with deuterium.

The compound represented by formula 3 may be at least one selected from the following compounds, but is not limited thereto.

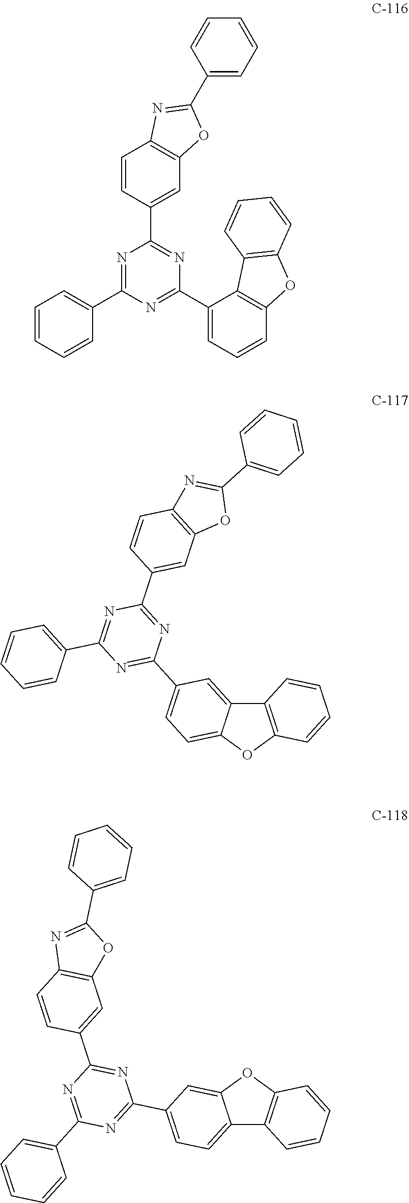 [SMILES: c1ccc(-c2nc(-c3ccc4nc(-c5ccccc5)oc4c3)nc(-c3cccc4oc5ccccc5c34)n2)cc1 ; c1ccc(-c2nc(-c3ccc4nc(-c5ccccc5)oc4c3)nc(-c3ccc4oc5ccccc5c4c3)n2)cc1 ; c1ccc(-c2nc(-c3ccc4nc(-c5ccccc5)oc4c3)nc(-c3ccc4c(c3)oc3ccccc34)n2)cc1]

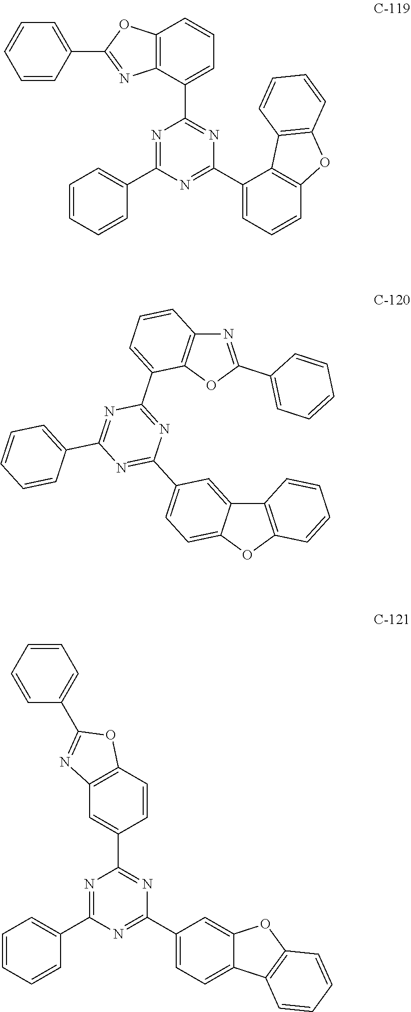 [SMILES: c1ccc(-c2nc(-c3cccc4oc(-c5ccccc5)nc34)nc(-c3cccc4oc5ccccc5c34)n2)cc1 ; c1ccc(-c2nc(-c3ccc4oc5ccccc5c4c3)nc(-c3cccc4nc(-c5ccccc5)oc34)n2)cc1 ; c1ccc(-c2nc(-c3ccc4oc(-c5ccccc5)nc4c3)nc(-c3ccc4c(c3)oc3ccccc34)n2)cc1]

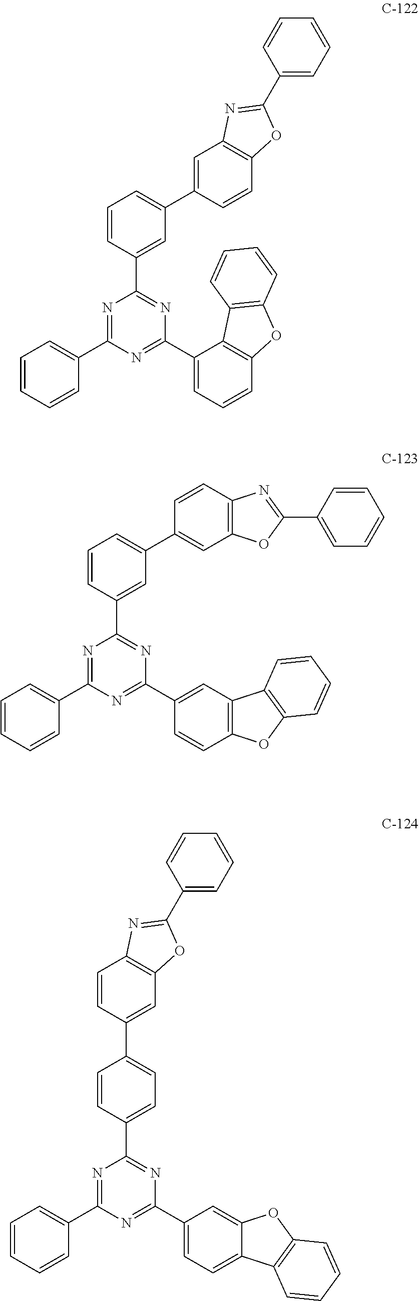 [SMILES: c1ccc(-c2nc(-c3cccc(-c4ccc5oc(-c6ccccc6)nc5c4)c3)nc(-c3cccc4oc5ccccc5c34)n2)cc1 ; c1ccc(-c2nc(-c3cccc(-c4ccc5nc(-c6ccccc6)oc5c4)c3)nc(-c3ccc4oc5ccccc5c4c3)n2)cc1 ; c1ccc(-c2nc(-c3ccc(-c4ccc5nc(-c6ccccc6)oc5c4)cc3)nc(-c3ccc4c(c3)oc3ccccc34)n2)cc1]

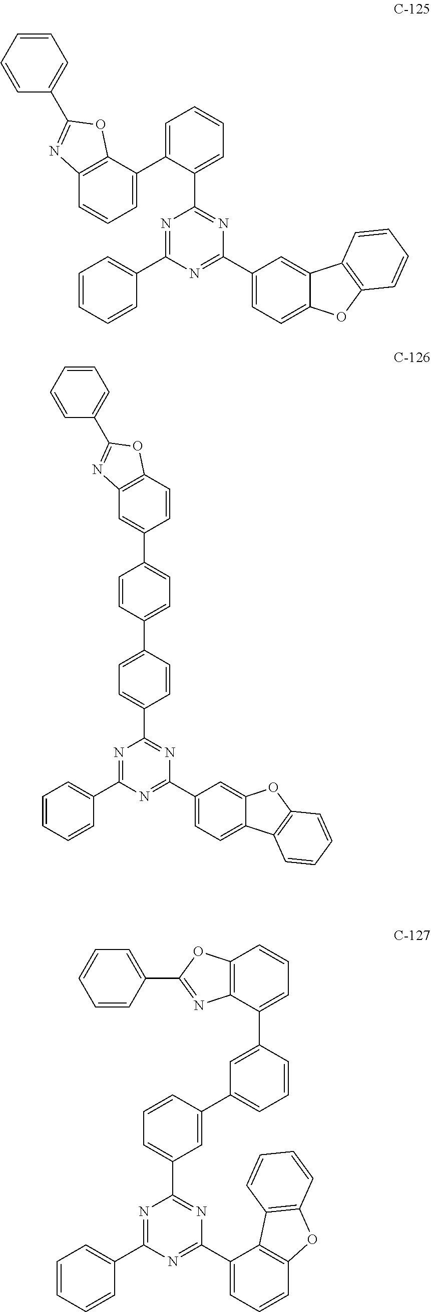

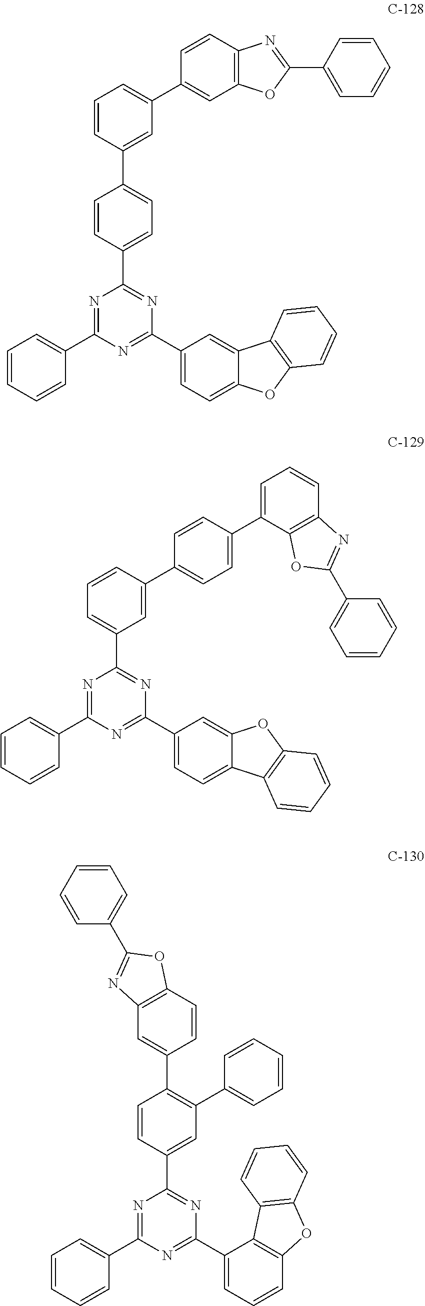 [SMILES: c1ccc(-c2nc(-c3ccc(-c4cccc(-c5ccc6nc(-c7ccccc7)oc6c5)c4)cc3)nc(-c3ccc4oc5ccccc5c4c3)n2)cc1 ; c1ccc(-c2nc(-c3cccc(-c4ccc(-c5cccc6nc(-c7ccccc7)oc56)cc4)c3)nc(-c3ccc4c(c3)oc3ccccc34)n2)cc1 ; c1ccc(-c2nc(-c3ccc(-c4ccc5oc(-c6ccccc6)nc5c4)c(-c4ccccc4)c3)nc(-c3cccc4oc5ccccc5c34)n2)cc1]

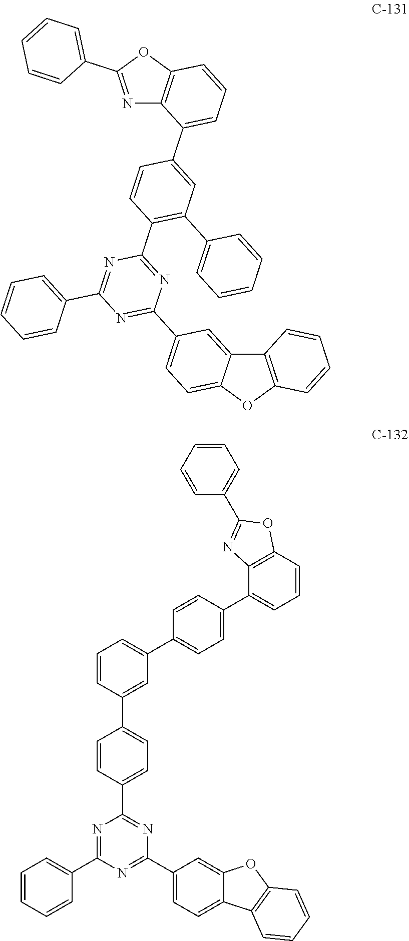 [SMILES: c1ccc(-c2nc(-c3ccc4oc5ccccc5c4c3)nc(-c3ccc(-c4cccc5oc(-c6ccccc6)nc45)cc3-c3ccccc3)n2)cc1 ; c1ccc(-c2nc(-c3ccc(-c4cccc(-c5ccc(-c6cccc7oc(-c8ccccc8)nc67)cc5)c4)cc3)nc(-c3ccc4c(c3)oc3ccccc34)n2)cc1]

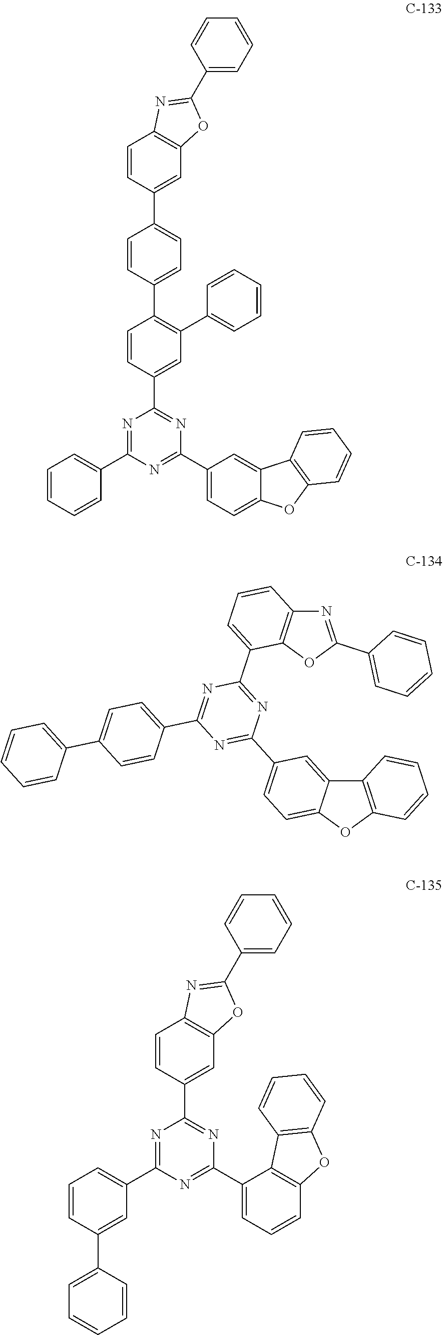 [SMILES: c1ccc(-c2nc(-c3ccc(-c4ccc(-c5ccc6nc(-c7ccccc7)oc6c5)cc4)c(-c4ccccc4)c3)nc(-c3ccc4oc5ccccc5c4c3)n2)cc1 ; c1ccc(-c2ccc(-c3nc(-c4ccc5oc6ccccc6c5c4)nc(-c4cccc5nc(-c6ccccc6)oc45)n3)cc2)cc1 ; c1ccc(-c2cccc(-c3nc(-c4ccc5nc(-c6ccccc6)oc5c4)nc(-c4cccc5oc6ccccc6c45)n3)c2)cc1]

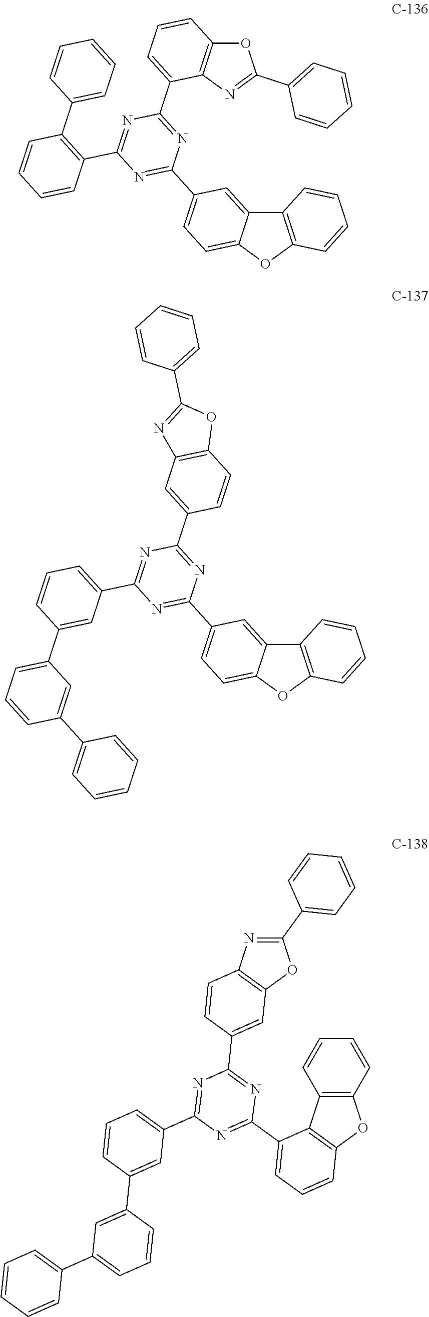 [SMILES: c1ccc(-c2nc3c(-c4nc(-c5ccc6oc7ccccc7c6c5)nc(-c5ccccc5-c5ccccc5)n4)cccc3o2)cc1 ; c1ccc(-c2cccc(-c3cccc(-c4nc(-c5ccc6oc(-c7ccccc7)nc6c5)nc(-c5ccc6oc7ccccc7c6c5)n4)c3)c2)cc1 ; c1ccc(-c2cccc(-c3cccc(-c4nc(-c5ccc6nc(-c7ccccc7)oc6c5)nc(-c5cccc6oc7ccccc7c56)n4)c3)c2)cc1]

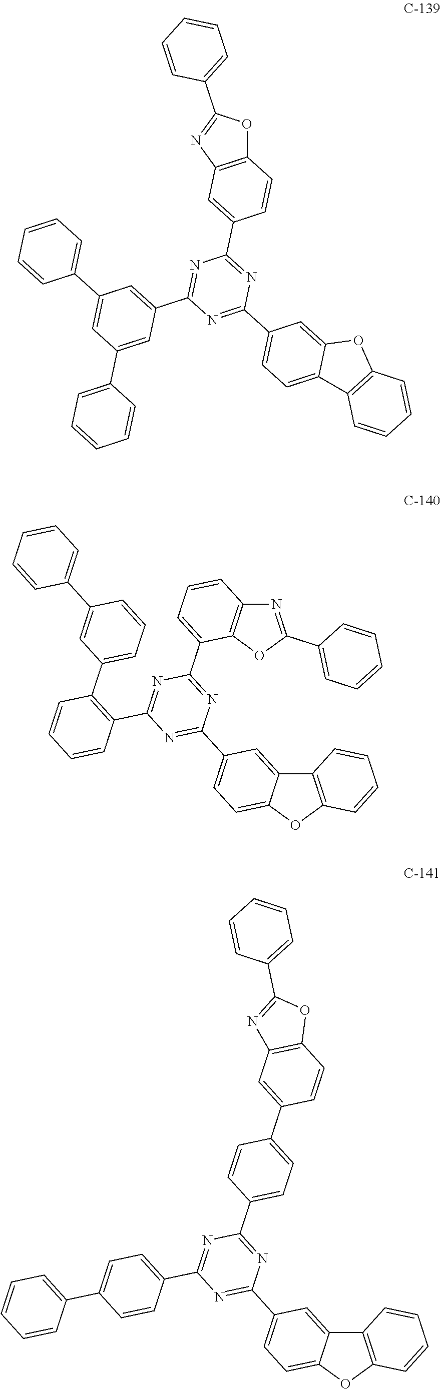 [SMILES: c1ccc(-c2cc(-c3ccccc3)cc(-c3nc(-c4ccc5oc(-c6ccccc6)nc5c4)nc(-c4ccc5c(c4)oc4ccccc45)n3)c2)cc1 ; c1ccc(-c2cccc(-c3ccccc3-c3nc(-c4ccc5oc6ccccc6c5c4)nc(-c4cccc5nc(-c6ccccc6)oc45)n3)c2)cc1 ; c1ccc(-c2ccc(-c3nc(-c4ccc(-c5ccc6oc(-c7ccccc7)nc6c5)cc4)nc(-c4ccc5oc6ccccc6c5c4)n3)cc2)cc1]

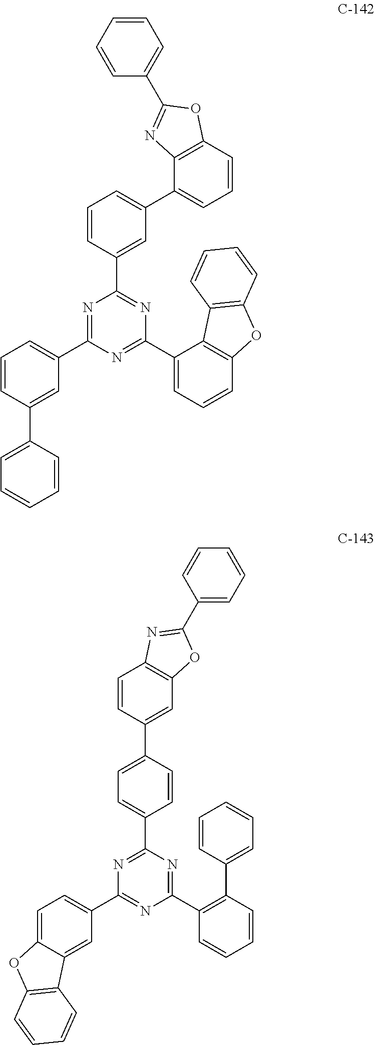 [SMILES: c1ccc(-c2cccc(-c3nc(-c4cccc(-c5cccc6oc(-c7ccccc7)nc56)c4)nc(-c4cccc5oc6ccccc6c45)n3)c2)cc1 ; c1ccc(-c2nc3ccc(-c4ccc(-c5nc(-c6ccc7oc8ccccc8c7c6)nc(-c6ccccc6-c6ccccc6)n5)cc4)cc3o2)cc1]

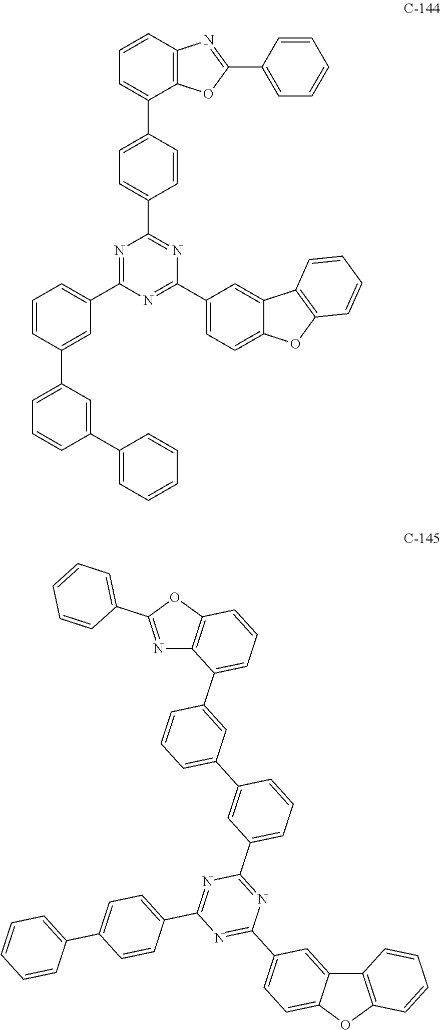 [SMILES: c1ccc(-c2cccc(-c3cccc(-c4nc(-c5ccc(-c6cccc7nc(-c8ccccc8)oc67)cc5)nc(-c5ccc6oc7ccccc7c6c5)n4)c3)c2)cc1 ; c1ccc(-c2ccc(-c3nc(-c4cccc(-c5cccc(-c6cccc7oc(-c8ccccc8)nc67)c5)c4)nc(-c4ccc5oc6ccccc6c5c4)n3)cc2)cc1]

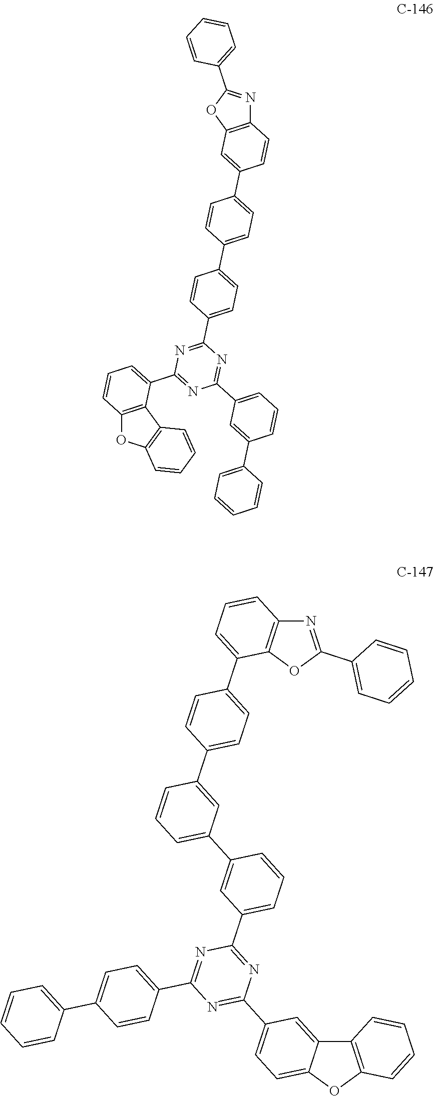 [SMILES: c1ccc(-c2cccc(-c3nc(-c4ccc(-c5ccc(-c6ccc7nc(-c8ccccc8)oc7c6)cc5)cc4)nc(-c4cccc5oc6ccccc6c45)n3)c2)cc1 ; c1ccc(-c2ccc(-c3nc(-c4cccc(-c5cccc(-c6ccc(-c7cccc8nc(-c9ccccc9)oc78)cc6)c5)c4)nc(-c4ccc5oc6ccccc6c5c4)n3)cc2)cc1]

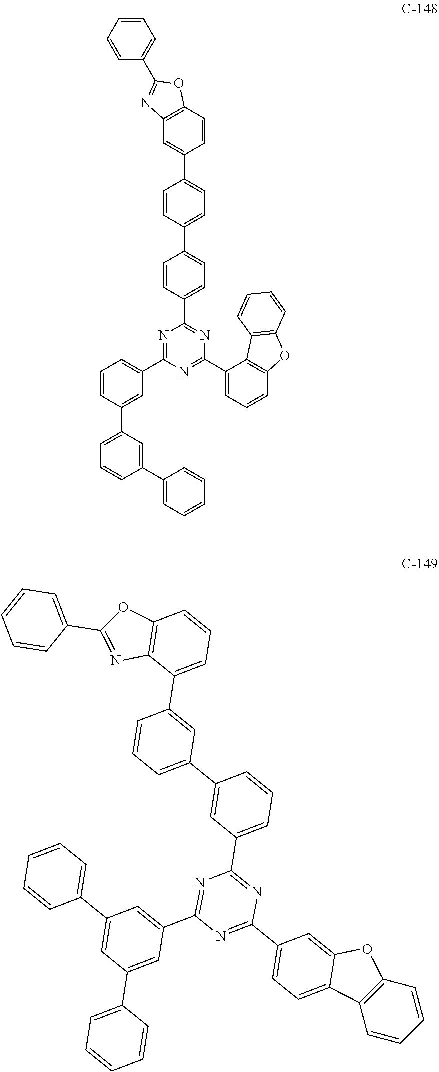 [SMILES: c1ccc(-c2cccc(-c3cccc(-c4nc(-c5ccc(-c6ccc(-c7ccc8oc(-c9ccccc9)nc8c7)cc6)cc5)nc(-c5cccc6oc7ccccc7c56)n4)c3)c2)cc1 ; c1ccc(-c2cc(-c3ccccc3)cc(-c3nc(-c4cccc(-c5cccc(-c6cccc7oc(-c8ccccc8)nc67)c5)c4)nc(-c4ccc5c(c4)oc4ccccc45)n3)c2)cc1]

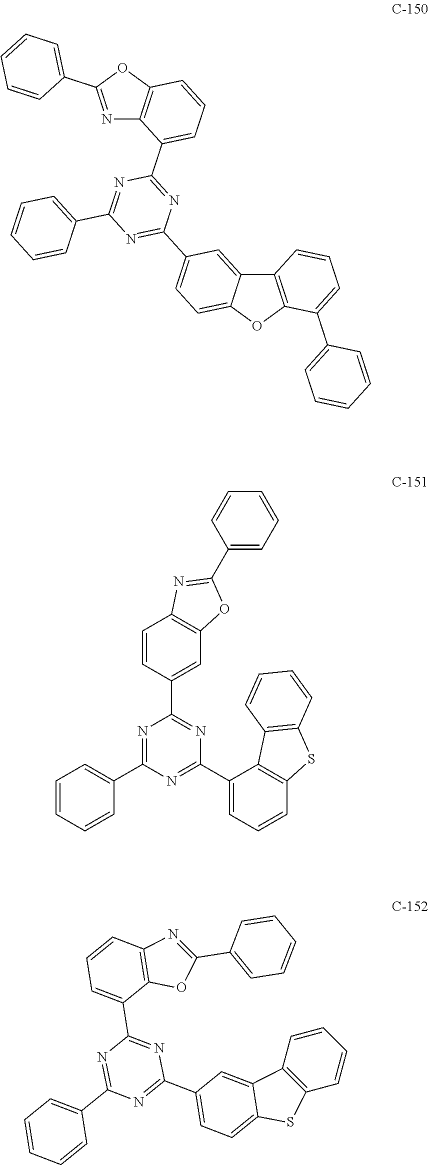 [SMILES: c1ccc(-c2nc(-c3ccc4oc5c(-c6ccccc6)cccc5c4c3)nc(-c3cccc4oc(-c5ccccc5)nc34)n2)cc1 ; c1ccc(-c2nc(-c3ccc4nc(-c5ccccc5)oc4c3)nc(-c3cccc4sc5ccccc5c34)n2)cc1 ; c1ccc(-c2nc(-c3ccc4sc5ccccc5c4c3)nc(-c3cccc4nc(-c5ccccc5)oc34)n2)cc1]

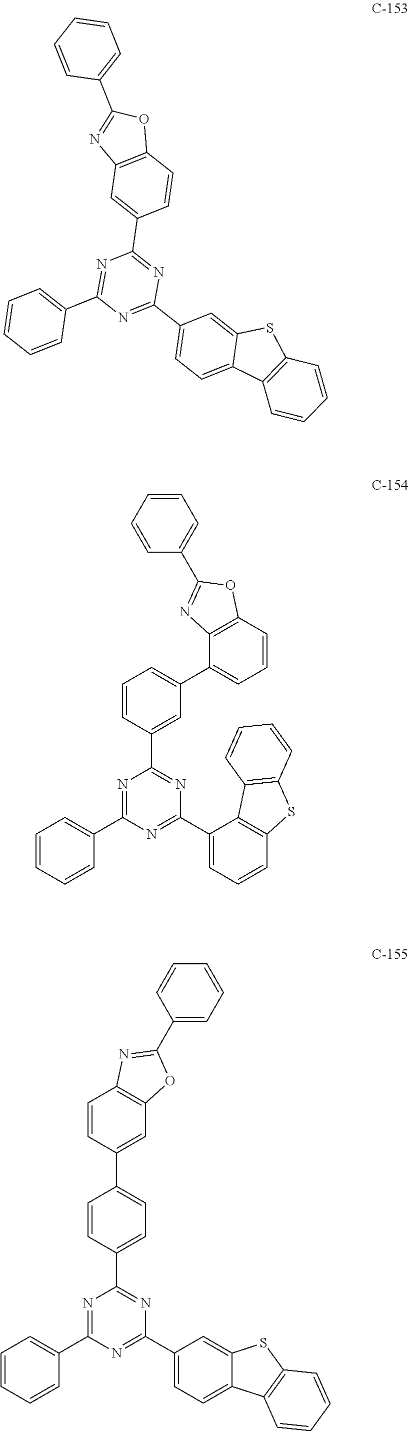 [SMILES: c1ccc(-c2nc(-c3ccc4oc(-c5ccccc5)nc4c3)nc(-c3ccc4c(c3)sc3ccccc34)n2)cc1 ; c1ccc(-c2nc(-c3cccc(-c4cccc5oc(-c6ccccc6)nc45)c3)nc(-c3cccc4sc5ccccc5c34)n2)cc1 ; c1ccc(-c2nc(-c3ccc(-c4ccc5nc(-c6ccccc6)oc5c4)cc3)nc(-c3ccc4c(c3)sc3ccccc34)n2)cc1]

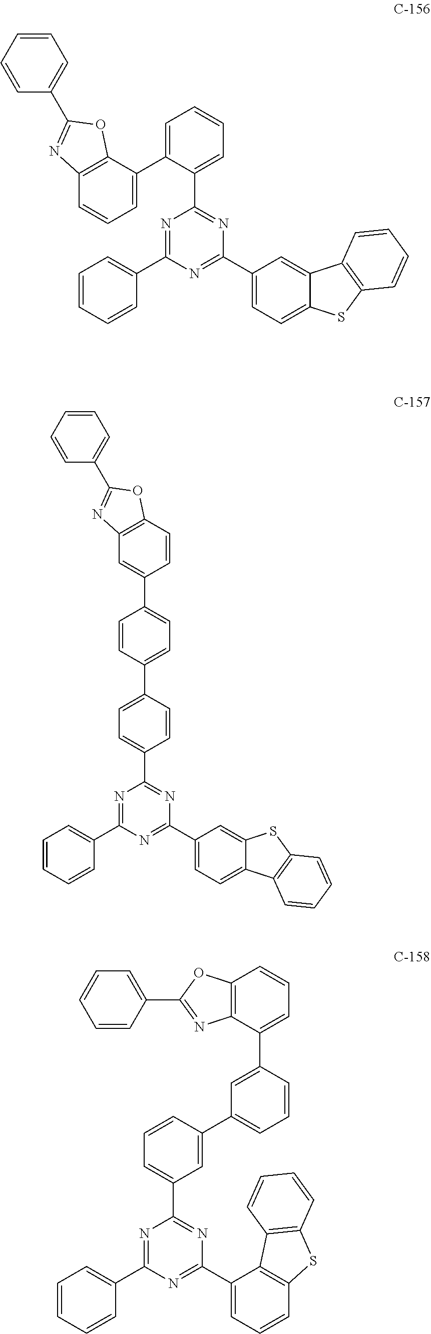 [SMILES: c1ccc(-c2nc(-c3ccc4sc5ccccc5c4c3)nc(-c3ccccc3-c3cccc4nc(-c5ccccc5)oc34)n2)cc1 ; c1ccc(-c2nc(-c3ccc(-c4ccc(-c5ccc6oc(-c7ccccc7)nc6c5)cc4)cc3)nc(-c3ccc4c(c3)sc3ccccc34)n2)cc1 ; c1ccc(-c2nc(-c3cccc(-c4cccc(-c5cccc6oc(-c7ccccc7)nc56)c4)c3)nc(-c3cccc4sc5ccccc5c34)n2)cc1]

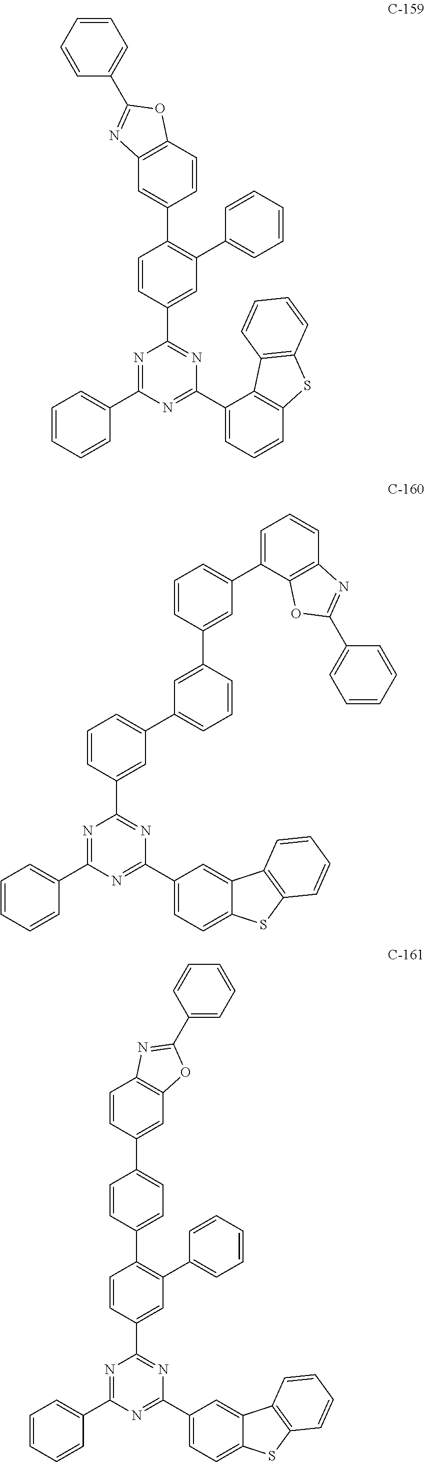

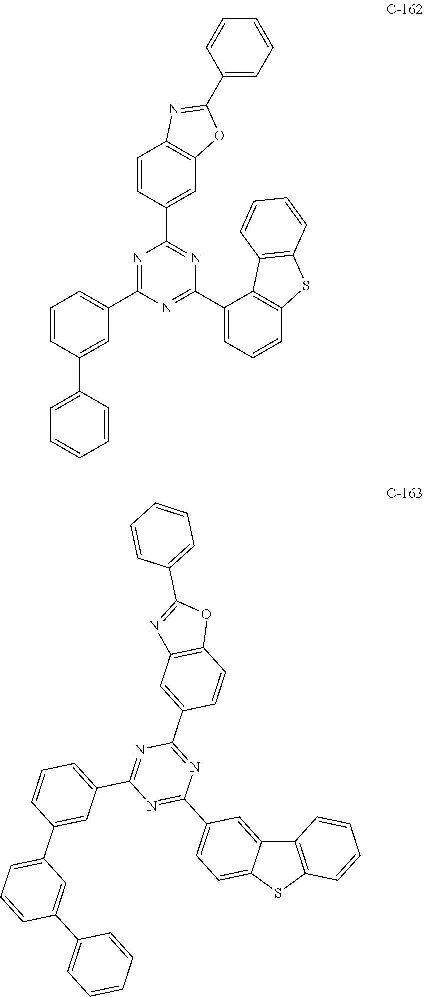 [SMILES: c1ccc(-c2cccc(-c3nc(-c4ccc5nc(-c6ccccc6)oc5c4)nc(-c4cccc5sc6ccccc6c45)n3)c2)cc1 ; c1ccc(-c2cccc(-c3cccc(-c4nc(-c5ccc6oc(-c7ccccc7)nc6c5)nc(-c5ccc6sc7ccccc7c6c5)n4)c3)c2)cc1]

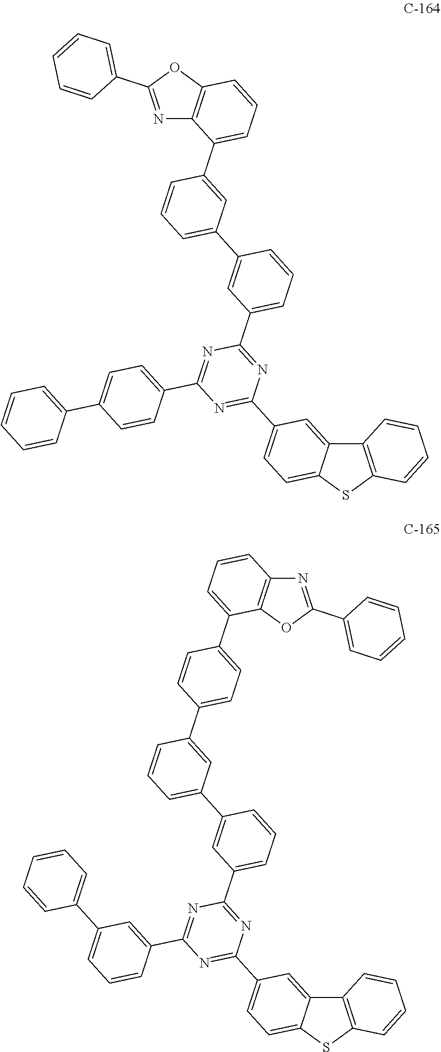 [SMILES: c1ccc(-c2ccc(-c3nc(-c4cccc(-c5cccc(-c6cccc7oc(-c8ccccc8)nc67)c5)c4)nc(-c4ccc5sc6ccccc6c5c4)n3)cc2)cc1 ; c1ccc(-c2cccc(-c3nc(-c4cccc(-c5cccc(-c6ccc(-c7cccc8nc(-c9ccccc9)oc78)cc6)c5)c4)nc(-c4ccc5sc6ccccc6c5c4)n3)c2)cc1]

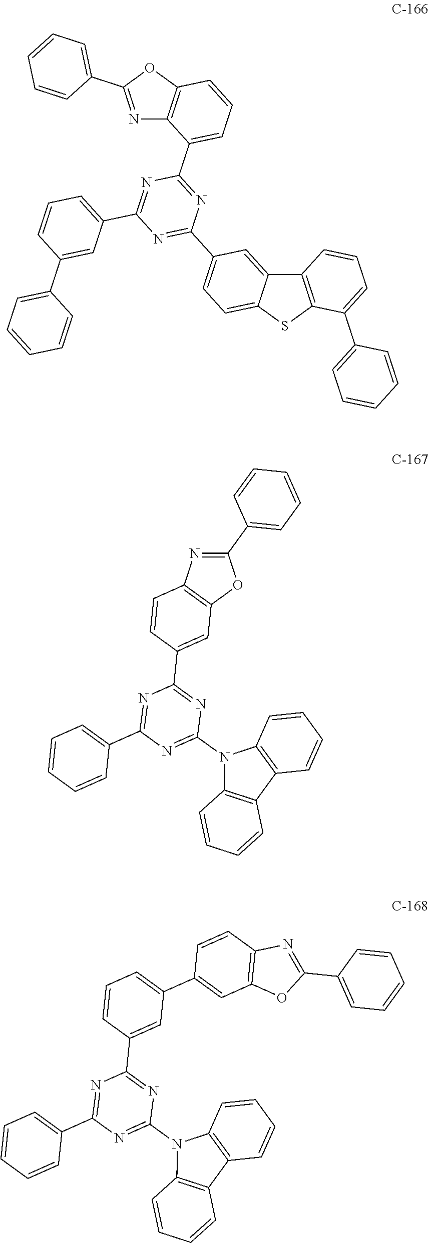 [SMILES: c1ccc(-c2cccc(-c3nc(-c4ccc5sc6c(-c7ccccc7)cccc6c5c4)nc(-c4cccc5oc(-c6ccccc6)nc45)n3)c2)cc1 ; c1ccc(-c2nc(-c3ccc4nc(-c5ccccc5)oc4c3)nc(-n3c4ccccc4c4ccccc43)n2)cc1 ; c1ccc(-c2nc(-c3cccc(-c4ccc5nc(-c6ccccc6)oc5c4)c3)nc(-n3c4ccccc4c4ccccc43)n2)cc1]

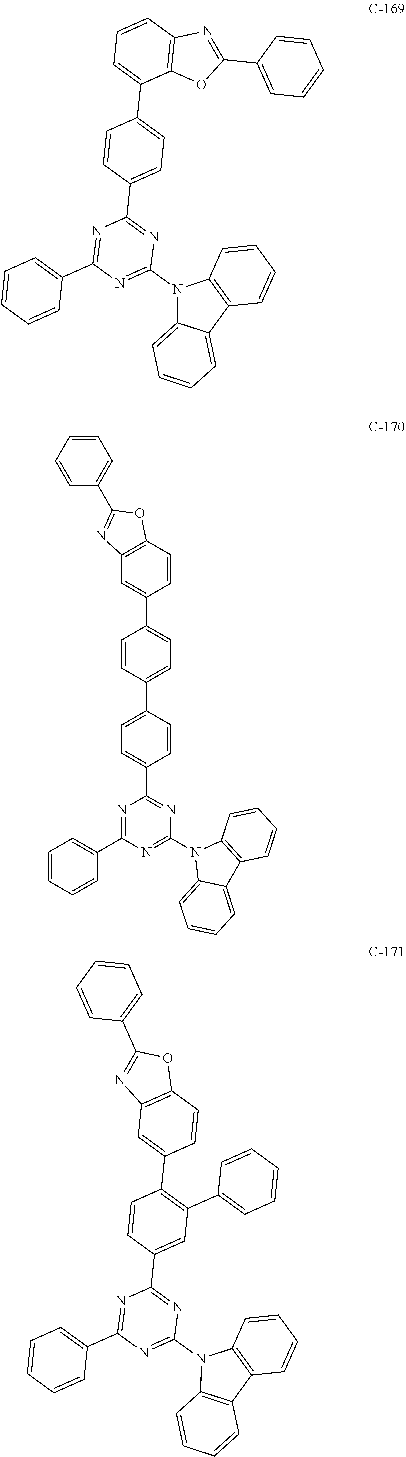 [SMILES: c1ccc(-c2nc(-c3ccc(-c4cccc5nc(-c6ccccc6)oc45)cc3)nc(-n3c4ccccc4c4ccccc43)n2)cc1 ; c1ccc(-c2nc(-c3ccc(-c4ccc(-c5ccc6oc(-c7ccccc7)nc6c5)cc4)cc3)nc(-n3c4ccccc4c4ccccc43)n2)cc1 ; c1ccc(-c2nc(-c3ccc(-c4ccc5oc(-c6ccccc6)nc5c4)c(-c4ccccc4)c3)nc(-n3c4ccccc4c4ccccc43)n2)cc1]

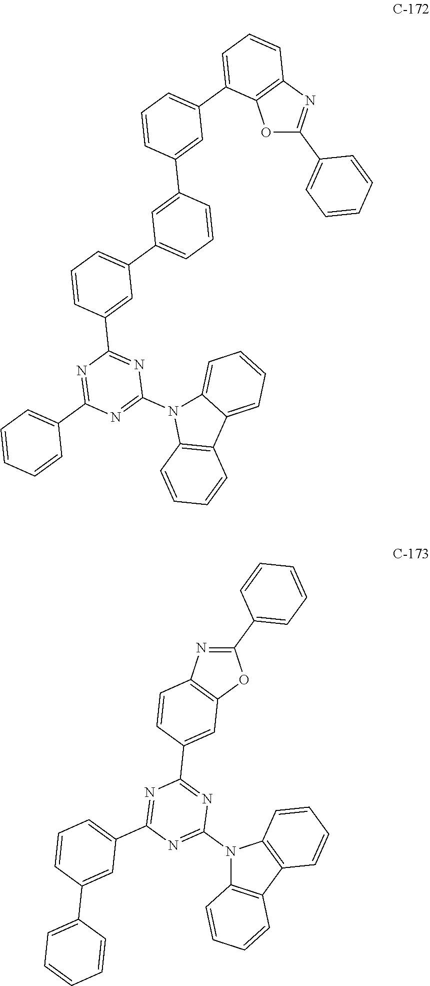 [SMILES: c1ccc(-c2nc(-c3cccc(-c4cccc(-c5cccc(-c6cccc7nc(-c8ccccc8)oc67)c5)c4)c3)nc(-n3c4ccccc4c4ccccc43)n2)cc1 ; c1ccc(-c2cccc(-c3nc(-c4ccc5nc(-c6ccccc6)oc5c4)nc(-n4c5ccccc5c5ccccc54)n3)c2)cc1]

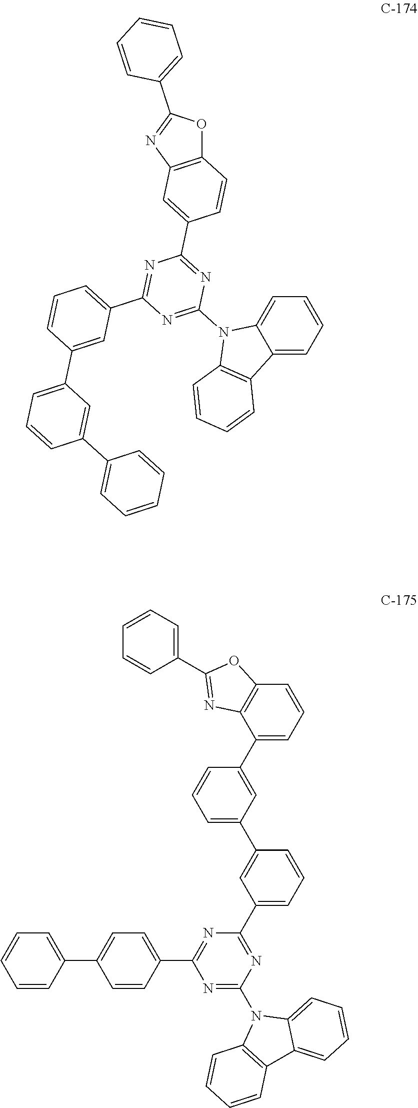 [SMILES: c1ccc(-c2cccc(-c3cccc(-c4nc(-c5ccc6oc(-c7ccccc7)nc6c5)nc(-n5c6ccccc6c6ccccc65)n4)c3)c2)cc1 ; c1ccc(-c2ccc(-c3nc(-c4cccc(-c5cccc(-c6cccc7oc(-c8ccccc8)nc67)c5)c4)nc(-n4c5ccccc5c5ccccc54)n3)cc2)cc1]

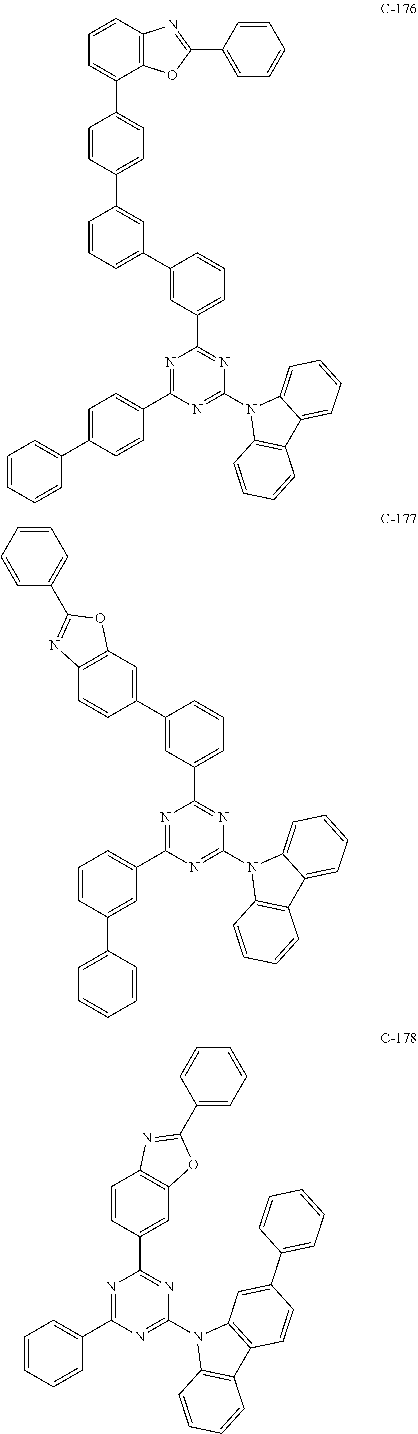

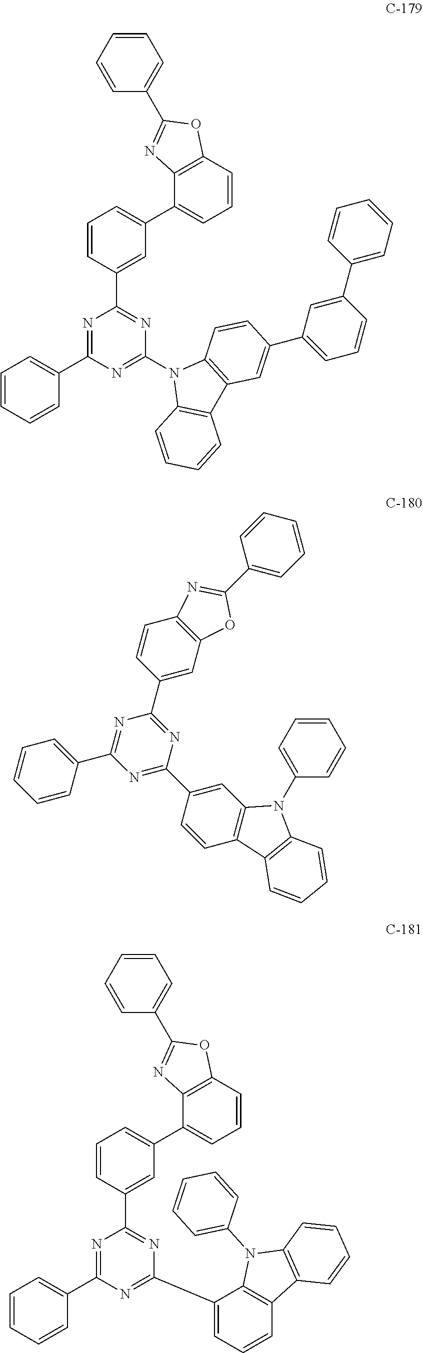 [SMILES: c1ccc(-c2cccc(-c3ccc4c(c3)c3ccccc3n4-c3nc(-c4ccccc4)nc(-c4cccc(-c5cccc6oc(-c7ccccc7)nc56)c4)n3)c2)cc1 ; c1ccc(-c2nc(-c3ccc4nc(-c5ccccc5)oc4c3)nc(-c3ccc4c5ccccc5n(-c5ccccc5)c4c3)n2)cc1 ; c1ccc(-c2nc(-c3cccc(-c4cccc5oc(-c6ccccc6)nc45)c3)nc(-c3cccc4c5ccccc5n(-c5ccccc5)c34)n2)cc1]

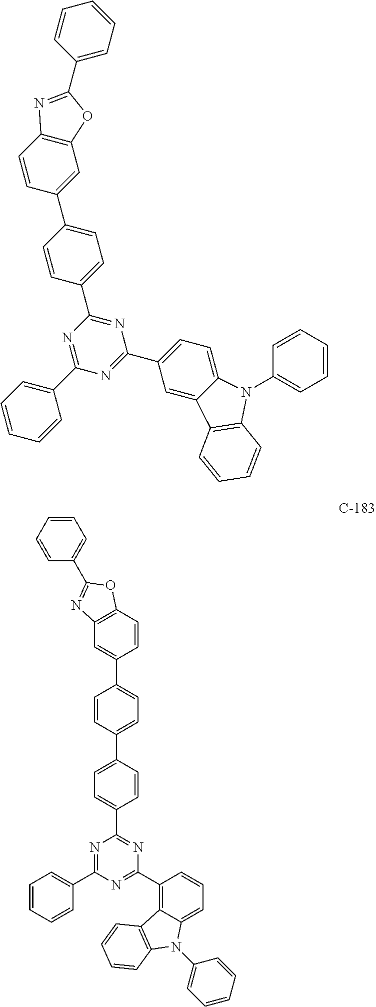 [SMILES: c1ccc(-c2nc(-c3ccc(-c4ccc5nc(-c6ccccc6)oc5c4)cc3)nc(-c3ccc4c(c3)c3ccccc3n4-c3ccccc3)n2)cc1 ; c1ccc(-c2nc(-c3ccc(-c4ccc(-c5ccc6oc(-c7ccccc7)nc6c5)cc4)cc3)nc(-c3cccc4c3c3ccccc3n4-c3ccccc3)n2)cc1]

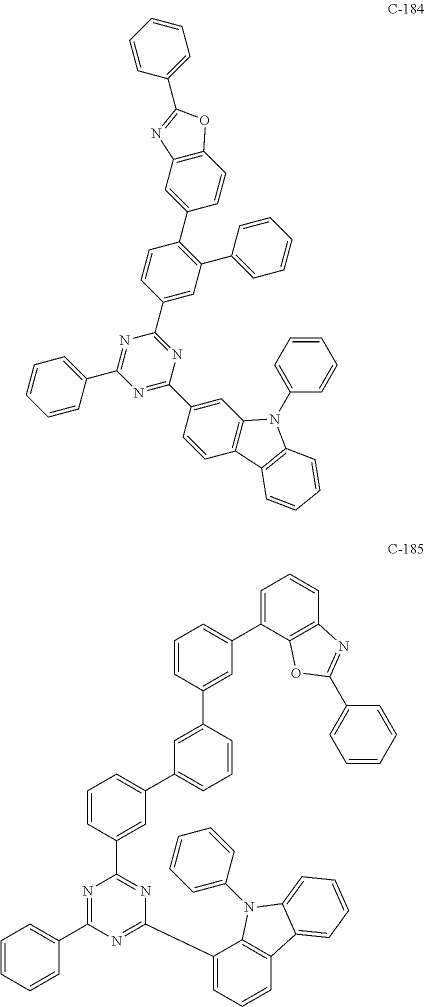 [SMILES: c1ccc(-c2nc(-c3ccc(-c4ccc5oc(-c6ccccc6)nc5c4)c(-c4ccccc4)c3)nc(-c3ccc4c5ccccc5n(-c5ccccc5)c4c3)n2)cc1 ; c1ccc(-c2nc(-c3cccc(-c4cccc(-c5cccc(-c6cccc7nc(-c8ccccc8)oc67)c5)c4)c3)nc(-c3cccc4c5ccccc5n(-c5ccccc5)c34)n2)cc1]

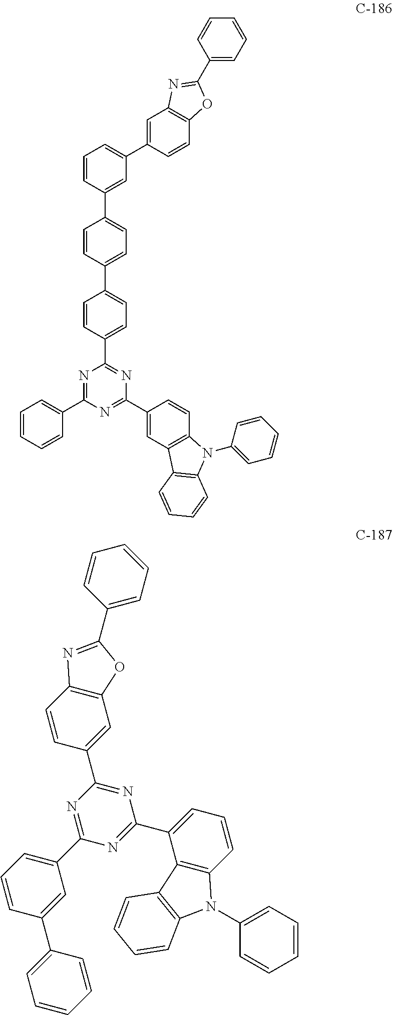 [SMILES: c1ccc(-c2nc(-c3ccc(-c4ccc(-c5cccc(-c6ccc7oc(-c8ccccc8)nc7c6)c5)cc4)cc3)nc(-c3ccc4c(c3)c3ccccc3n4-c3ccccc3)n2)cc1 ; c1ccc(-c2cccc(-c3nc(-c4ccc5nc(-c6ccccc6)oc5c4)nc(-c4cccc5c4c4ccccc4n5-c4ccccc4)n3)c2)cc1]

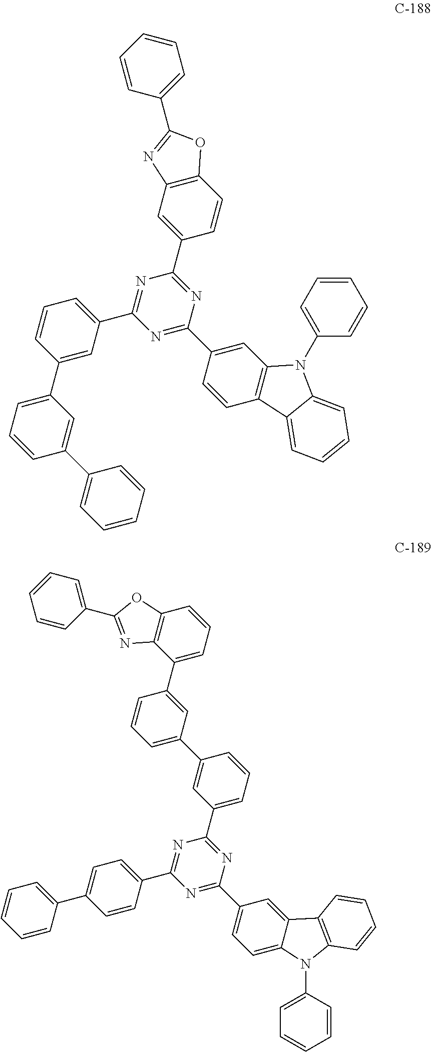 [SMILES: c1ccc(-c2cccc(-c3cccc(-c4nc(-c5ccc6oc(-c7ccccc7)nc6c5)nc(-c5ccc6c7ccccc7n(-c7ccccc7)c6c5)n4)c3)c2)cc1 ; c1ccc(-c2ccc(-c3nc(-c4cccc(-c5cccc(-c6cccc7oc(-c8ccccc8)nc67)c5)c4)nc(-c4ccc5c(c4)c4ccccc4n5-c4ccccc4)n3)cc2)cc1]

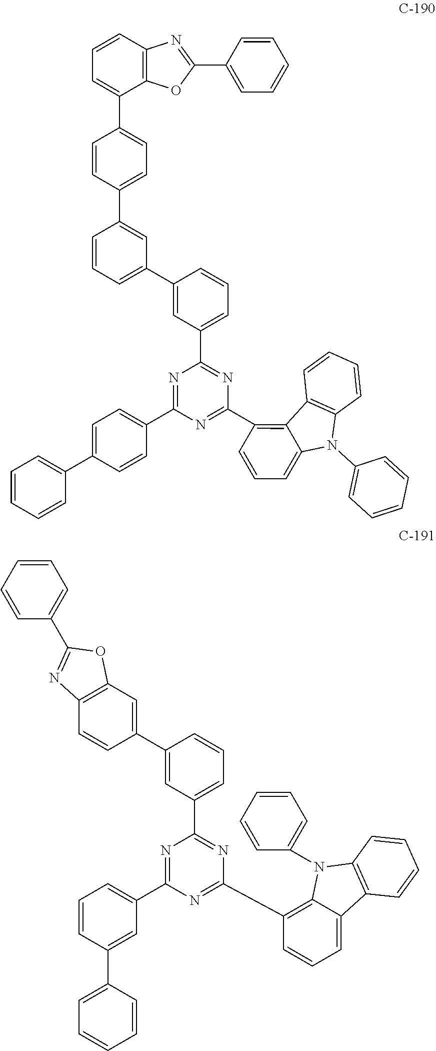 [SMILES: c1ccc(-c2ccc(-c3nc(-c4cccc(-c5cccc(-c6ccc(-c7cccc8nc(-c9ccccc9)oc78)cc6)c5)c4)nc(-c4cccc5c4c4ccccc4n5-c4ccccc4)n3)cc2)cc1 ; c1ccc(-c2cccc(-c3nc(-c4cccc(-c5ccc6nc(-c7ccccc7)oc6c5)c4)nc(-c4cccc5c6ccccc6n(-c6ccccc6)c45)n3)c2)cc1]

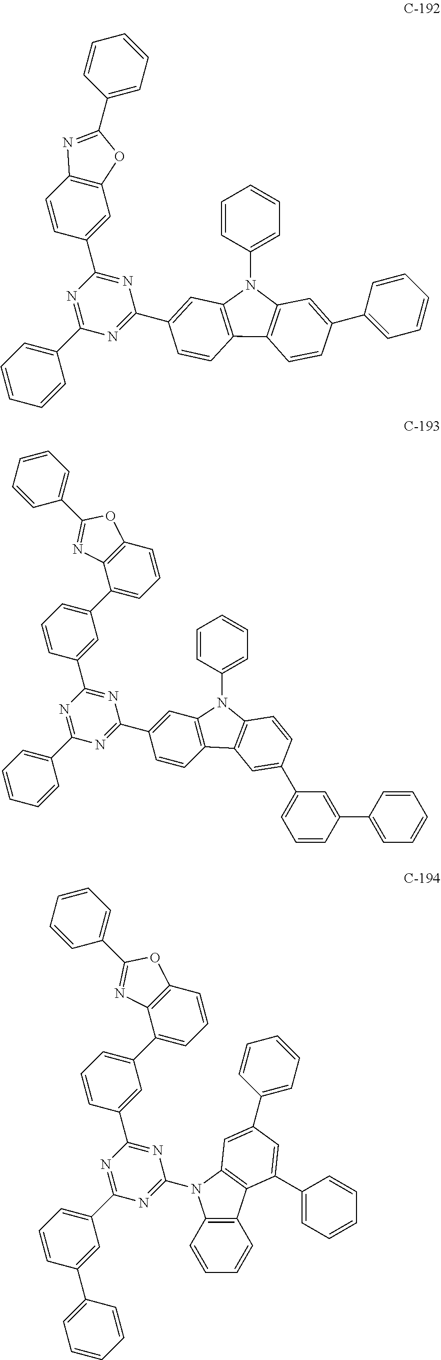 [SMILES: c1ccc(-c2ccc3c4ccc(-c5nc(-c6ccccc6)nc(-c6ccc7nc(-c8ccccc8)oc7c6)n5)cc4n(-c4ccccc4)c3c2)cc1 ; c1ccc(-c2cccc(-c3ccc4c(c3)c3ccc(-c5nc(-c6ccccc6)nc(-c6cccc(-c7cccc8oc(-c9ccccc9)nc78)c6)n5)cc3n4-c3ccccc3)c2)cc1 ; c1ccc(-c2cccc(-c3nc(-c4cccc(-c5cccc6oc(-c7ccccc7)nc56)c4)nc(-n4c5ccccc5c5c(-c6ccccc6)cc(-c6ccccc6)cc54)n3)c2)cc1]

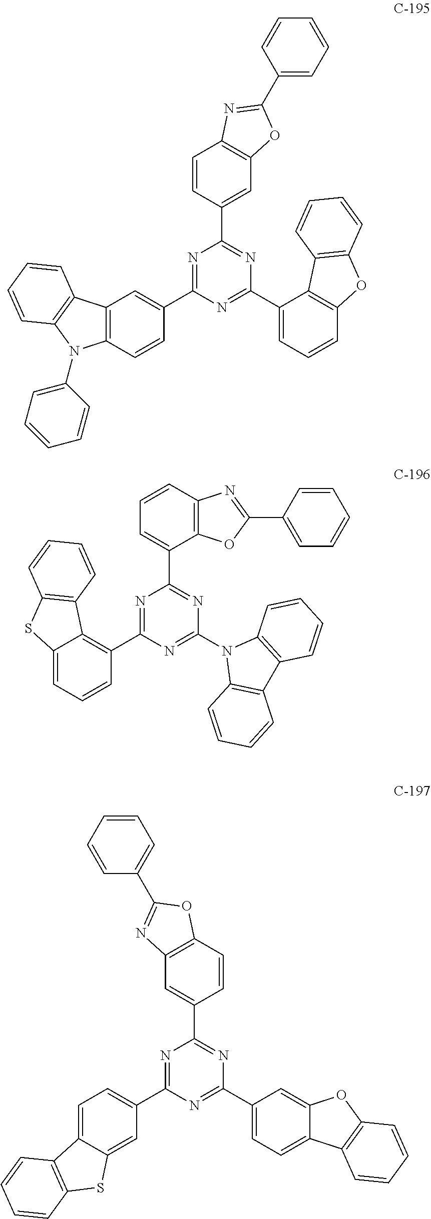 [SMILES: c1ccc(-c2nc3ccc(-c4nc(-c5ccc6c(c5)c5ccccc5n6-c5ccccc5)nc(-c5cccc6oc7ccccc7c56)n4)cc3o2)cc1 ; c1ccc(-c2nc3cccc(-c4nc(-c5cccc6sc7ccccc7c56)nc(-n5c6ccccc6c6ccccc65)n4)c3o2)cc1 ; c1ccc(-c2nc3cc(-c4nc(-c5ccc6c(c5)oc5ccccc56)nc(-c5ccc6c(c5)sc5ccccc56)n4)ccc3o2)cc1]

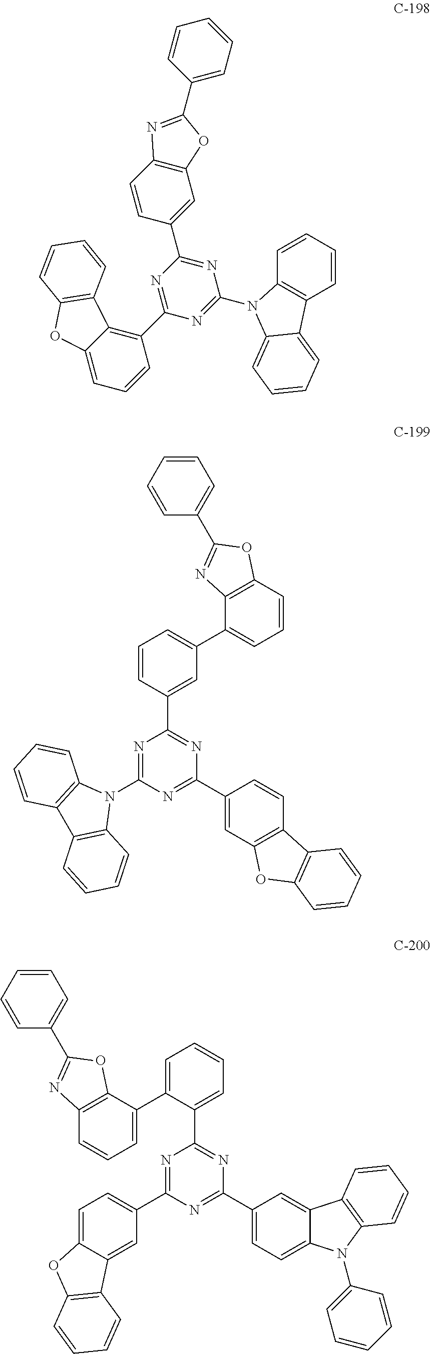 [SMILES: c1ccc(-c2nc3ccc(-c4nc(-c5cccc6oc7ccccc7c56)nc(-n5c6ccccc6c6ccccc65)n4)cc3o2)cc1 ; c1ccc(-c2nc3c(-c4cccc(-c5nc(-c6ccc7c(c6)oc6ccccc67)nc(-n6c7ccccc7c7ccccc76)n5)c4)cccc3o2)cc1 ; c1ccc(-c2nc3cccc(-c4ccccc4-c4nc(-c5ccc6oc7ccccc7c6c5)nc(-c5ccc6c(c5)c5ccccc5n6-c5ccccc5)n4)c3o2)cc1]

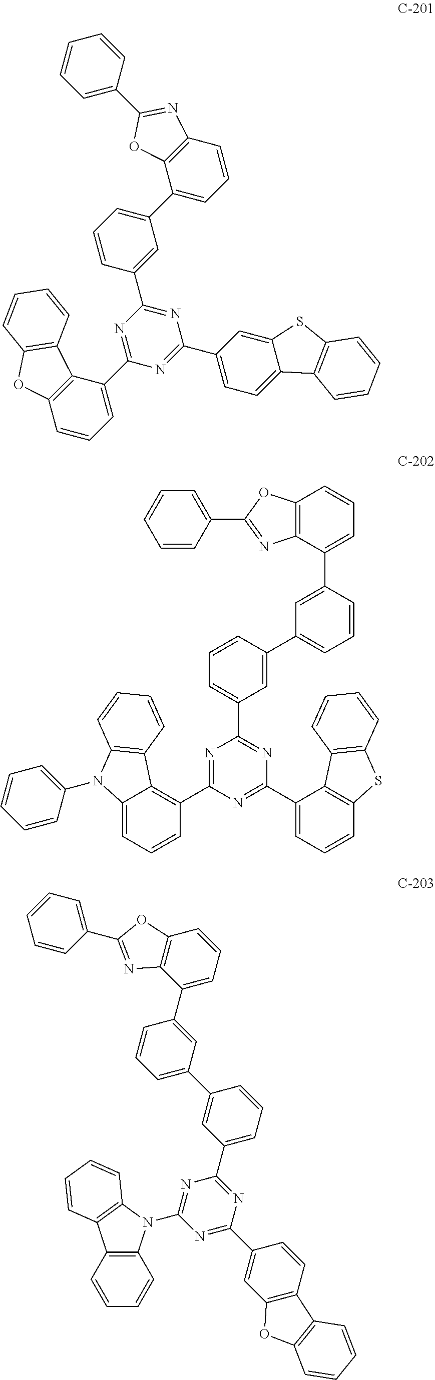 [SMILES: c1ccc(-c2nc3cccc(-c4cccc(-c5nc(-c6ccc7c(c6)sc6ccccc67)nc(-c6cccc7oc8ccccc8c67)n5)c4)c3o2)cc1 ; c1ccc(-c2nc3c(-c4cccc(-c5cccc(-c6nc(-c7cccc8sc9ccccc9c78)nc(-c7cccc8c7c7ccccc7n8-c7ccccc7)n6)c5)c4)cccc3o2)cc1 ; c1ccc(-c2nc3c(-c4cccc(-c5cccc(-c6nc(-c7ccc8c(c7)oc7ccccc78)nc(-n7c8ccccc8c8ccccc87)n6)c5)c4)cccc3o2)cc1]

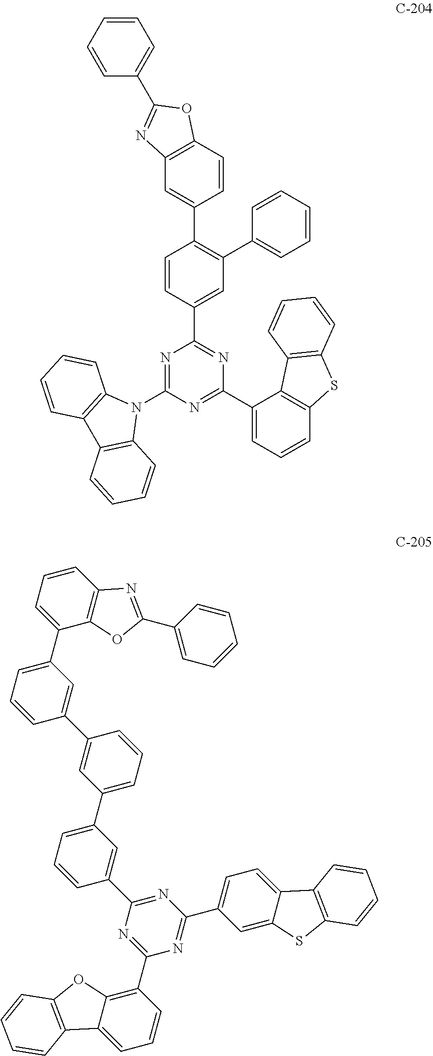 [SMILES: c1ccc(-c2nc3cc(-c4ccc(-c5nc(-c6cccc7sc8ccccc8c67)nc(-n6c7ccccc7c7ccccc76)n5)cc4-c4ccccc4)ccc3o2)cc1 ; c1ccc(-c2nc3cccc(-c4cccc(-c5cccc(-c6cccc(-c7nc(-c8ccc9c(c8)sc8ccccc89)nc(-c8cccc9c8oc8ccccc89)n7)c6)c5)c4)c3o2)cc1]

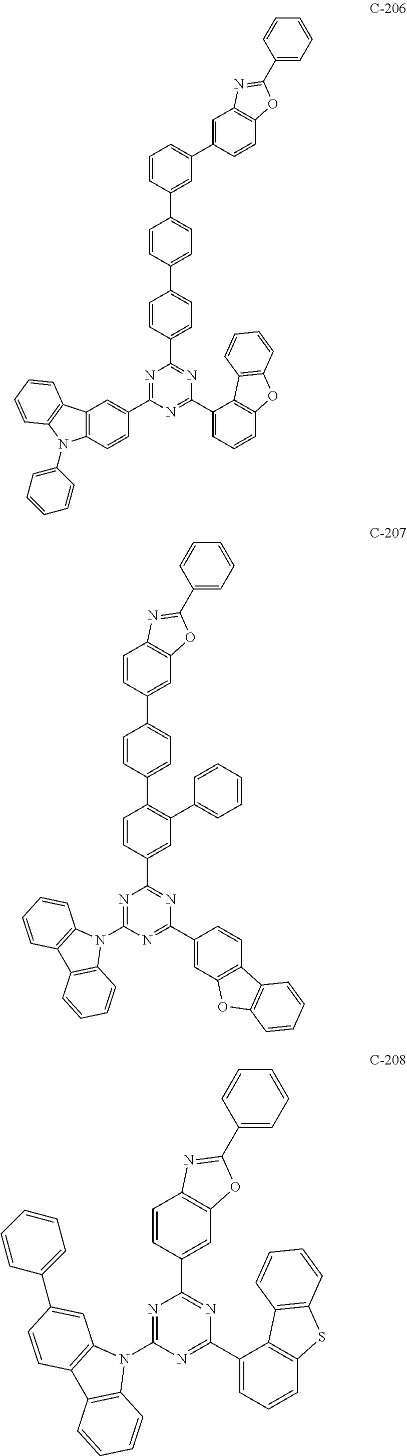 [SMILES: c1ccc(-c2nc3cc(-c4cccc(-c5ccc(-c6ccc(-c7nc(-c8ccc9c(c8)c8ccccc8n9-c8ccccc8)nc(-c8cccc9oc%10ccccc%10c89)n7)cc6)cc5)c4)ccc3o2)cc1 ; c1ccc(-c2nc3ccc(-c4ccc(-c5ccc(-c6nc(-c7ccc8c(c7)oc7ccccc78)nc(-n7c8ccccc8c8ccccc87)n6)cc5-c5ccccc5)cc4)cc3o2)cc1 ; c1ccc(-c2ccc3c4ccccc4n(-c4nc(-c5ccc6nc(-c7ccccc7)oc6c5)nc(-c5cccc6sc7ccccc7c56)n4)c3c2)cc1]

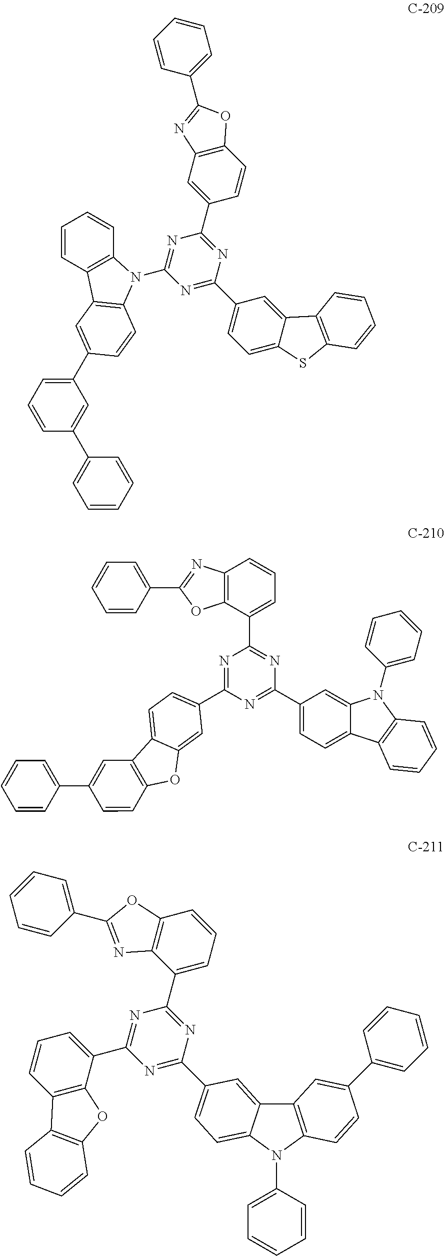 [SMILES: c1ccc(-c2cccc(-c3ccc4c(c3)c3ccccc3n4-c3nc(-c4ccc5oc(-c6ccccc6)nc5c4)nc(-c4ccc5sc6ccccc6c5c4)n3)c2)cc1 ; c1ccc(-c2ccc3oc4cc(-c5nc(-c6ccc7c8ccccc8n(-c8ccccc8)c7c6)nc(-c6cccc7nc(-c8ccccc8)oc67)n5)ccc4c3c2)cc1 ; c1ccc(-c2ccc3c(c2)c2cc(-c4nc(-c5cccc6oc(-c7ccccc7)nc56)nc(-c5cccc6c5oc5ccccc56)n4)ccc2n3-c2ccccc2)cc1]

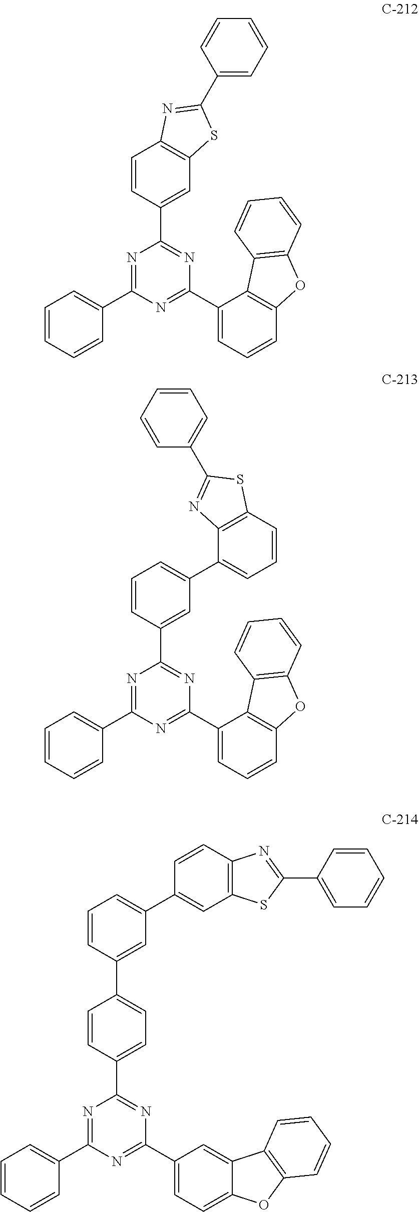 [SMILES: c1ccc(-c2nc(-c3ccc4nc(-c5ccccc5)sc4c3)nc(-c3cccc4oc5ccccc5c34)n2)cc1 ; c1ccc(-c2nc(-c3cccc(-c4cccc5sc(-c6ccccc6)nc45)c3)nc(-c3cccc4oc5ccccc5c34)n2)cc1 ; c1ccc(-c2nc(-c3ccc(-c4cccc(-c5ccc6nc(-c7ccccc7)sc6c5)c4)cc3)nc(-c3ccc4oc5ccccc5c4c3)n2)cc1]

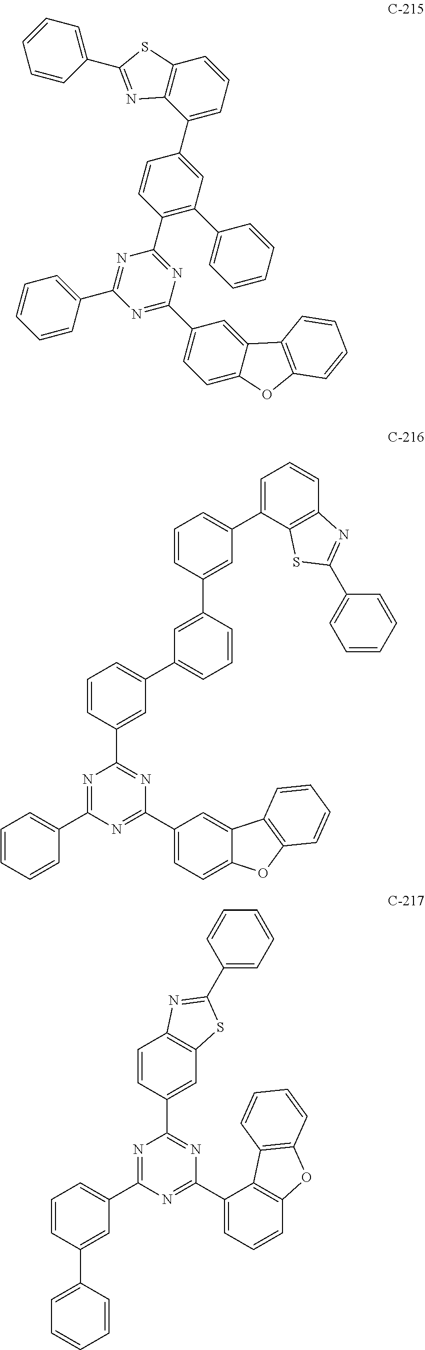

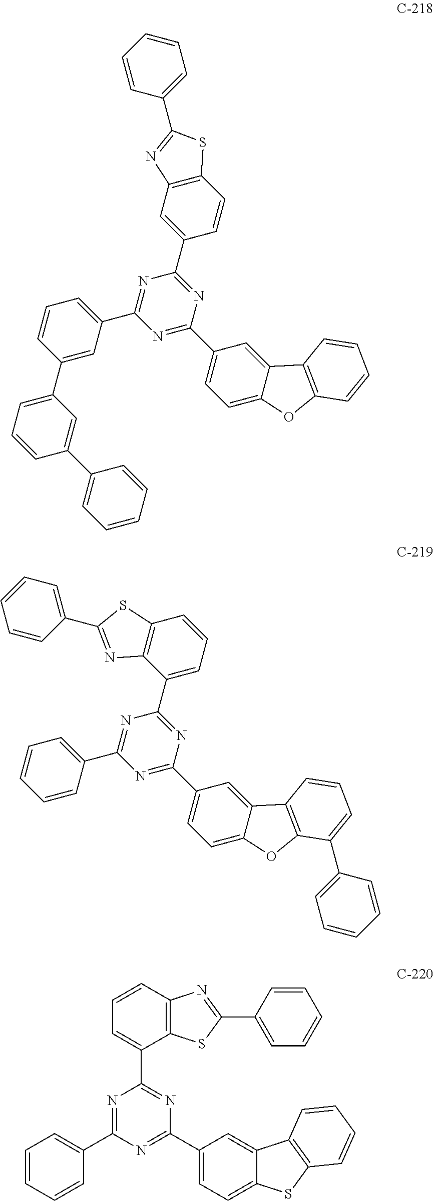 [SMILES: c1ccc(-c2cccc(-c3cccc(-c4nc(-c5ccc6sc(-c7ccccc7)nc6c5)nc(-c5ccc6oc7ccccc7c6c5)n4)c3)c2)cc1 ; c1ccc(-c2nc(-c3ccc4oc5c(-c6ccccc6)cccc5c4c3)nc(-c3cccc4sc(-c5ccccc5)nc34)n2)cc1 ; c1ccc(-c2nc(-c3ccc4sc5ccccc5c4c3)nc(-c3cccc4nc(-c5ccccc5)sc34)n2)cc1]

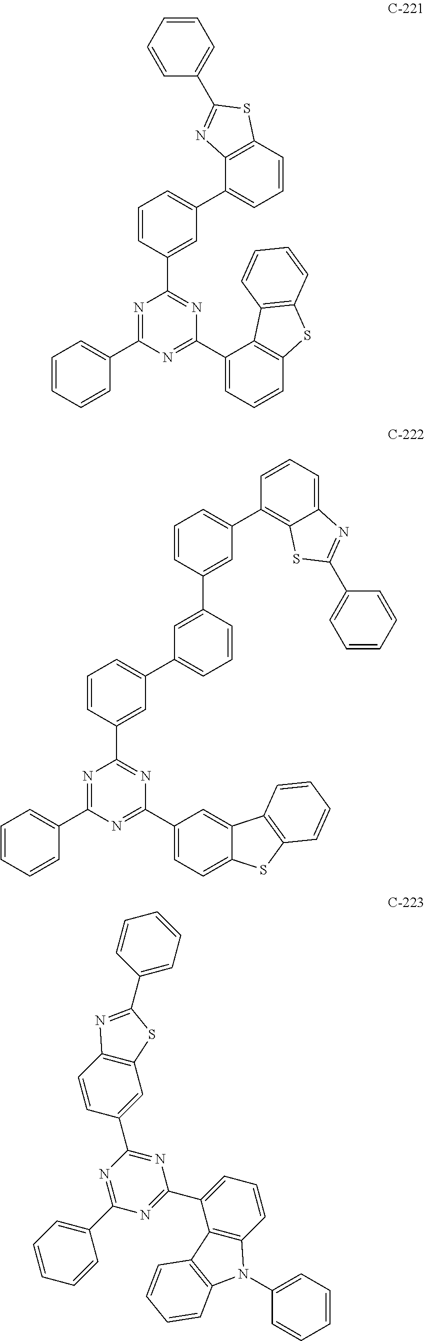 [SMILES: c1ccc(-c2nc(-c3cccc(-c4cccc5sc(-c6ccccc6)nc45)c3)nc(-c3cccc4sc5ccccc5c34)n2)cc1 ; c1ccc(-c2nc(-c3cccc(-c4cccc(-c5cccc(-c6cccc7nc(-c8ccccc8)sc67)c5)c4)c3)nc(-c3ccc4sc5ccccc5c4c3)n2)cc1 ; c1ccc(-c2nc(-c3ccc4nc(-c5ccccc5)sc4c3)nc(-c3cccc4c3c3ccccc3n4-c3ccccc3)n2)cc1]

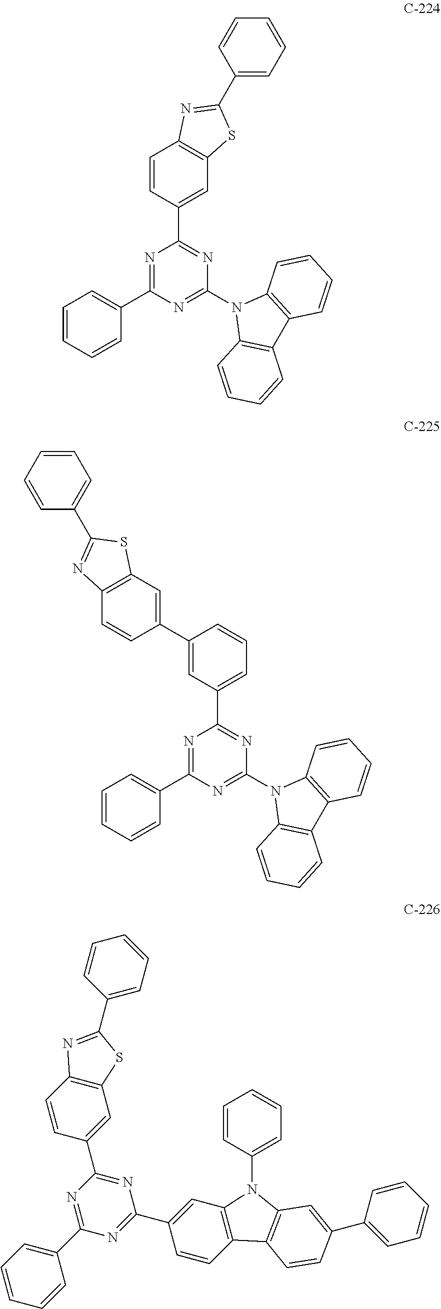 [SMILES: c1ccc(-c2nc(-c3ccc4nc(-c5ccccc5)sc4c3)nc(-n3c4ccccc4c4ccccc43)n2)cc1 ; c1ccc(-c2nc(-c3cccc(-c4ccc5nc(-c6ccccc6)sc5c4)c3)nc(-n3c4ccccc4c4ccccc43)n2)cc1 ; c1ccc(-c2ccc3c4ccc(-c5nc(-c6ccccc6)nc(-c6ccc7nc(-c8ccccc8)sc7c6)n5)cc4n(-c4ccccc4)c3c2)cc1]

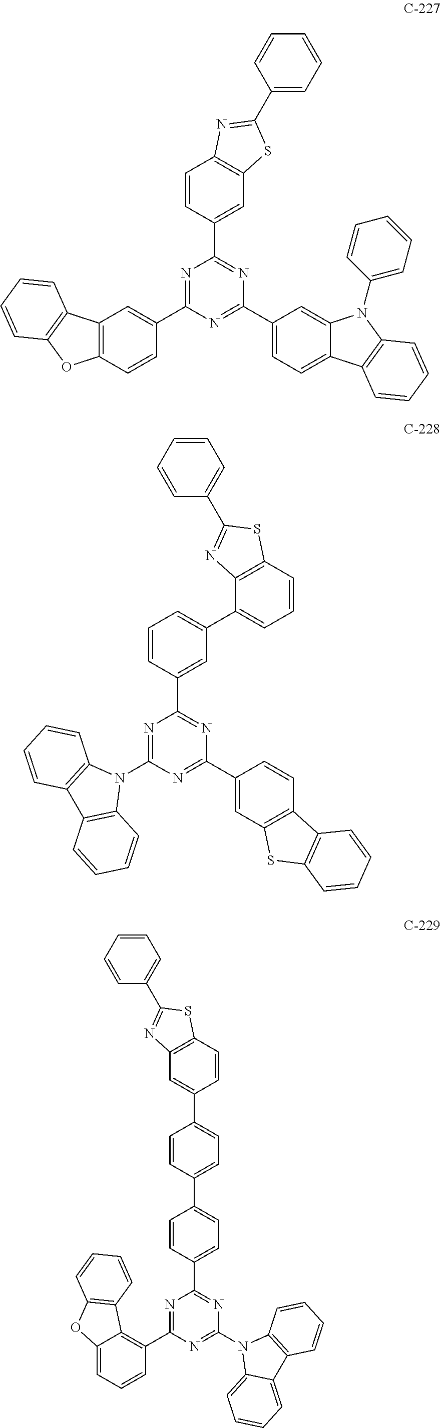 [SMILES: c1ccc(-c2nc3ccc(-c4nc(-c5ccc6oc7ccccc7c6c5)nc(-c5ccc6c7ccccc7n(-c7ccccc7)c6c5)n4)cc3s2)cc1 ; c1ccc(-c2nc3c(-c4cccc(-c5nc(-c6ccc7c(c6)sc6ccccc67)nc(-n6c7ccccc7c7ccccc76)n5)c4)cccc3s2)cc1 ; c1ccc(-c2nc3cc(-c4ccc(-c5ccc(-c6nc(-c7cccc8oc9ccccc9c78)nc(-n7c8ccccc8c8ccccc87)n6)cc5)cc4)ccc3s2)cc1]

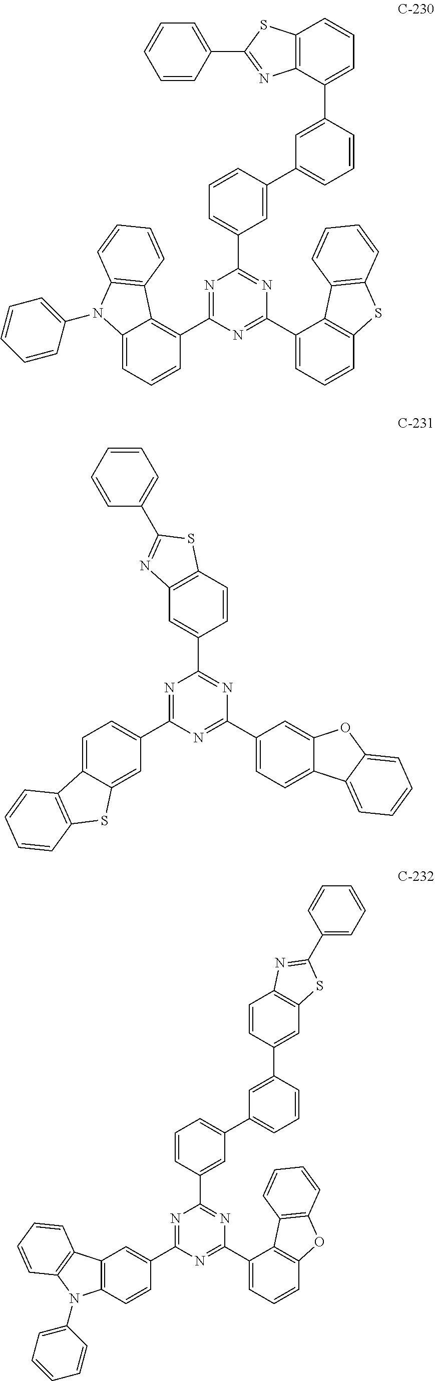 [SMILES: c1ccc(-c2nc3c(-c4cccc(-c5cccc(-c6nc(-c7cccc8sc9ccccc9c78)nc(-c7cccc8c7c7ccccc7n8-c7ccccc7)n6)c5)c4)cccc3s2)cc1 ; c1ccc(-c2nc3cc(-c4nc(-c5ccc6c(c5)oc5ccccc56)nc(-c5ccc6c(c5)sc5ccccc56)n4)ccc3s2)cc1 ; c1ccc(-c2nc3ccc(-c4cccc(-c5cccc(-c6nc(-c7ccc8c(c7)c7ccccc7n8-c7ccccc7)nc(-c7cccc8oc9ccccc9c78)n6)c5)c4)cc3s2)cc1]

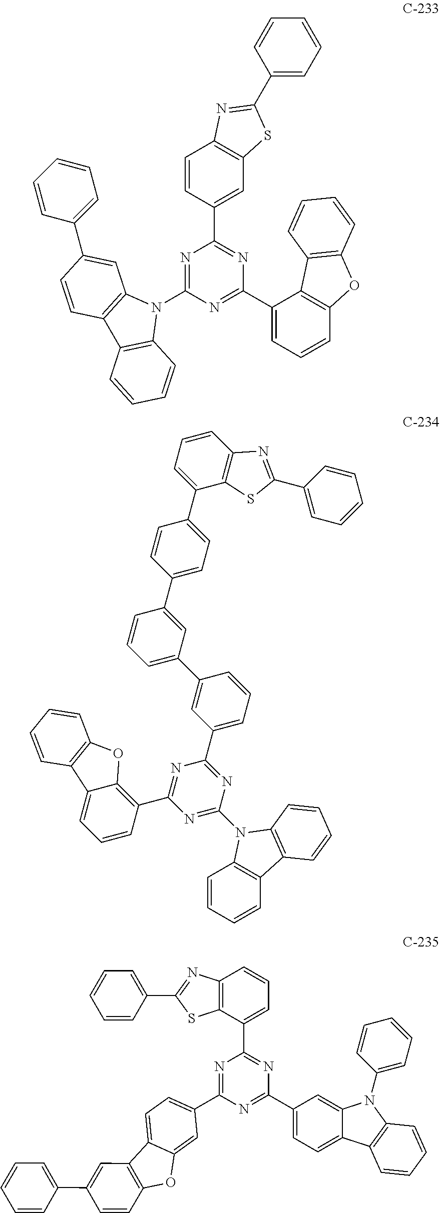 [SMILES: c1ccc(-c2ccc3c4ccccc4n(-c4nc(-c5ccc6nc(-c7ccccc7)sc6c5)nc(-c5cccc6oc7ccccc7c56)n4)c3c2)cc1 ; c1ccc(-c2nc3cccc(-c4ccc(-c5cccc(-c6cccc(-c7nc(-c8cccc9c8oc8ccccc89)nc(-n8c9ccccc9c9ccccc98)n7)c6)c5)cc4)c3s2)cc1 ; c1ccc(-c2ccc3oc4cc(-c5nc(-c6ccc7c8ccccc8n(-c8ccccc8)c7c6)nc(-c6cccc7nc(-c8ccccc8)sc67)n5)ccc4c3c2)cc1]

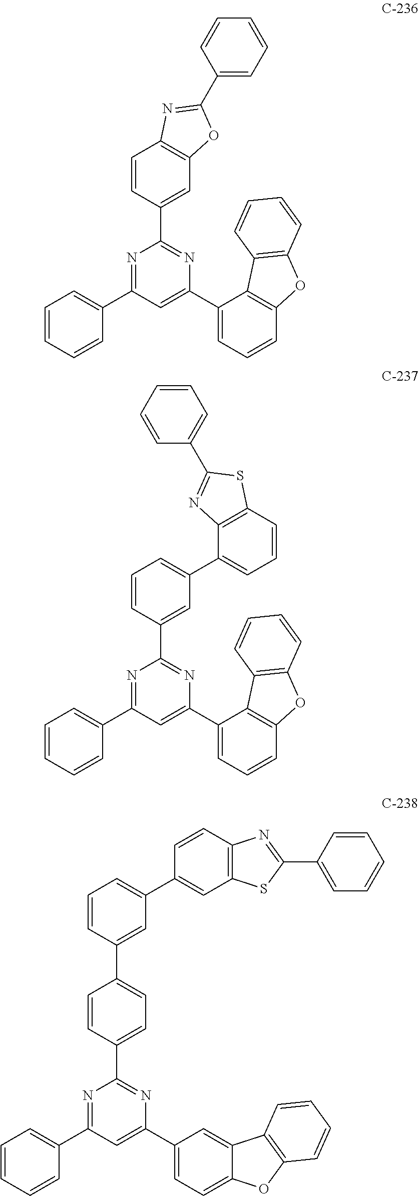 [SMILES: c1ccc(-c2cc(-c3cccc4oc5ccccc5c34)nc(-c3ccc4nc(-c5ccccc5)oc4c3)n2)cc1 ; c1ccc(-c2cc(-c3cccc4oc5ccccc5c34)nc(-c3cccc(-c4cccc5sc(-c6ccccc6)nc45)c3)n2)cc1 ; c1ccc(-c2cc(-c3ccc4oc5ccccc5c4c3)nc(-c3ccc(-c4cccc(-c5ccc6nc(-c7ccccc7)sc6c5)c4)cc3)n2)cc1]

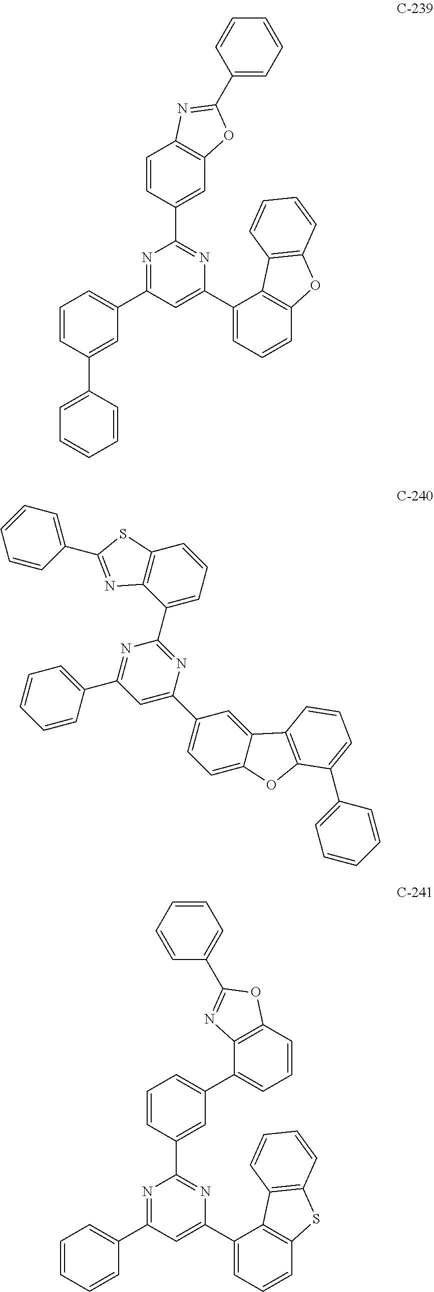 [SMILES: c1ccc(-c2cccc(-c3cc(-c4cccc5oc6ccccc6c45)nc(-c4ccc5nc(-c6ccccc6)oc5c4)n3)c2)cc1 ; c1ccc(-c2cc(-c3ccc4oc5c(-c6ccccc6)cccc5c4c3)nc(-c3cccc4sc(-c5ccccc5)nc34)n2)cc1 ; c1ccc(-c2cc(-c3cccc4sc5ccccc5c34)nc(-c3cccc(-c4cccc5oc(-c6ccccc6)nc45)c3)n2)cc1]

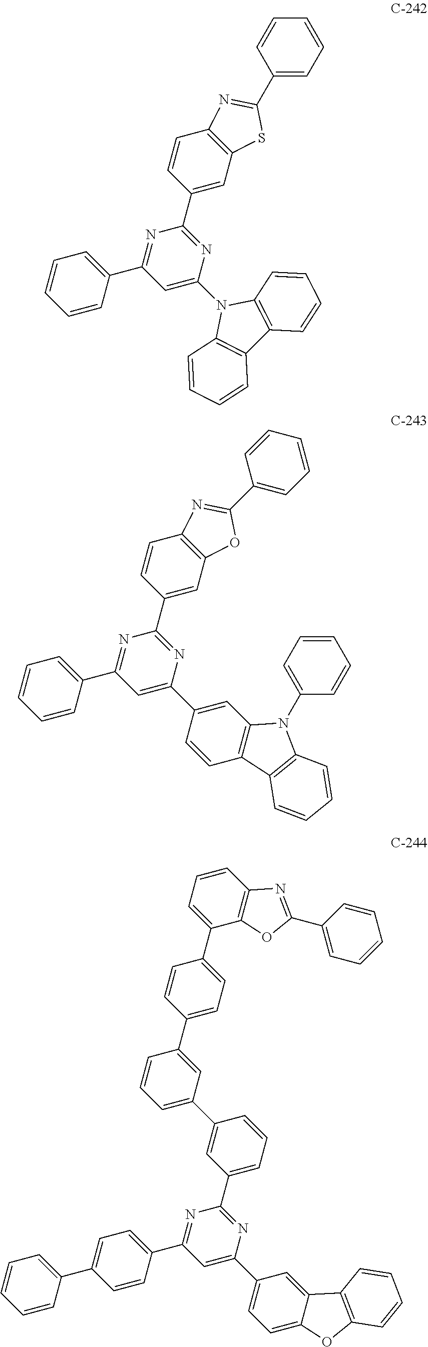 [SMILES: c1ccc(-c2cc(-n3c4ccccc4c4ccccc43)nc(-c3ccc4nc(-c5ccccc5)sc4c3)n2)cc1 ; c1ccc(-c2cc(-c3ccc4c5ccccc5n(-c5ccccc5)c4c3)nc(-c3ccc4nc(-c5ccccc5)oc4c3)n2)cc1 ; c1ccc(-c2ccc(-c3cc(-c4ccc5oc6ccccc6c5c4)nc(-c4cccc(-c5cccc(-c6ccc(-c7cccc8nc(-c9ccccc9)oc78)cc6)c5)c4)n3)cc2)cc1]

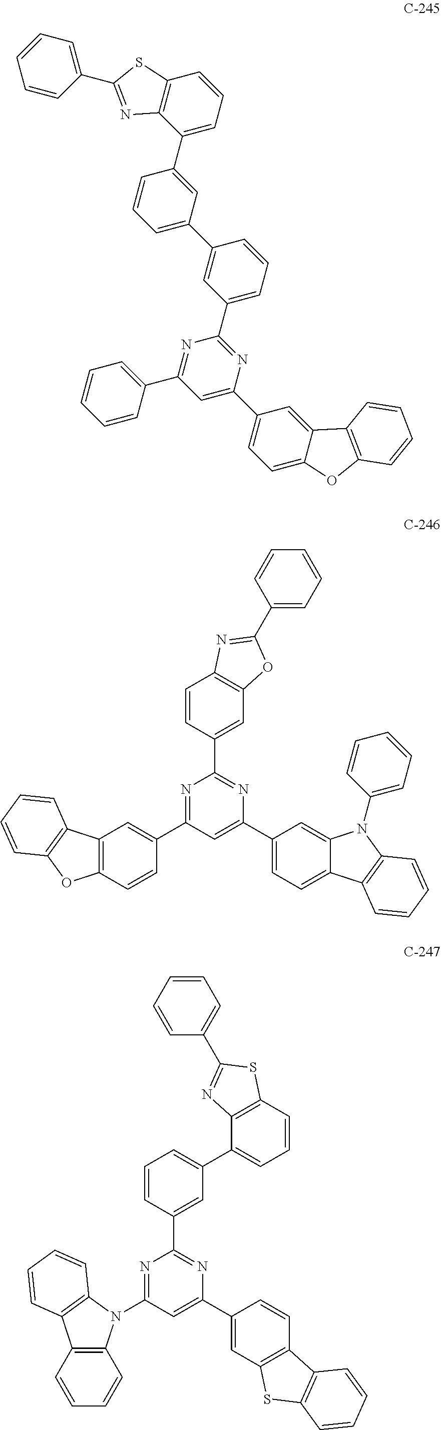 [SMILES: c1ccc(-c2cc(-c3ccc4oc5ccccc5c4c3)nc(-c3cccc(-c4cccc(-c5cccc6sc(-c7ccccc7)nc56)c4)c3)n2)cc1 ; c1ccc(-c2nc3ccc(-c4nc(-c5ccc6oc7ccccc7c6c5)cc(-c5ccc6c7ccccc7n(-c7ccccc7)c6c5)n4)cc3o2)cc1 ; c1ccc(-c2nc3c(-c4cccc(-c5nc(-c6ccc7c(c6)sc6ccccc67)cc(-n6c7ccccc7c7ccccc76)n5)c4)cccc3s2)cc1]

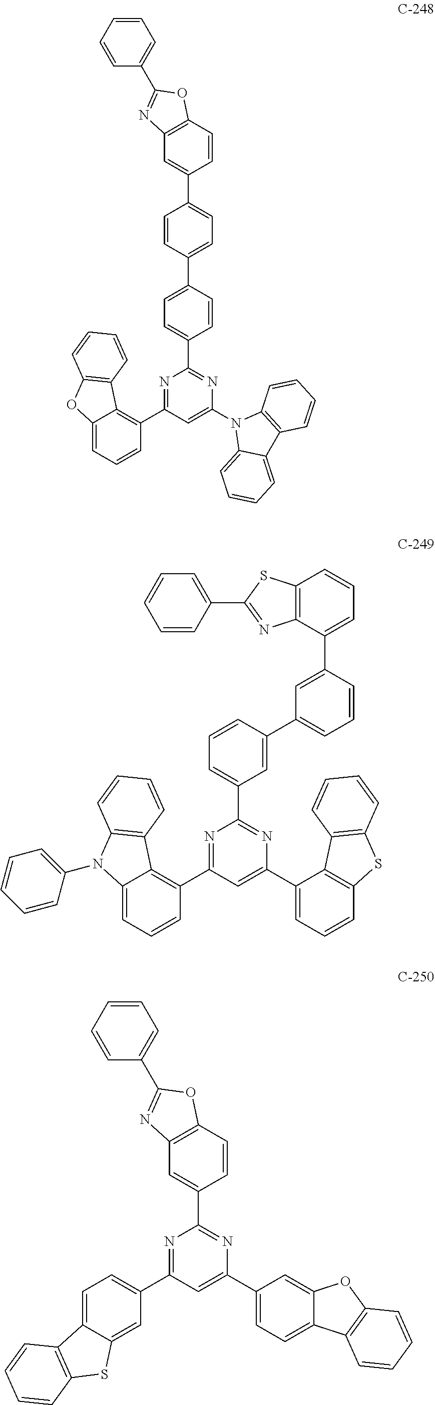 [SMILES: c1ccc(-c2nc3cc(-c4ccc(-c5ccc(-c6nc(-c7cccc8oc9ccccc9c78)cc(-n7c8ccccc8c8ccccc87)n6)cc5)cc4)ccc3o2)cc1 ; c1ccc(-c2nc3c(-c4cccc(-c5cccc(-c6nc(-c7cccc8sc9ccccc9c78)cc(-c7cccc8c7c7ccccc7n8-c7ccccc7)n6)c5)c4)cccc3s2)cc1 ; c1ccc(-c2nc3cc(-c4nc(-c5ccc6c(c5)oc5ccccc56)cc(-c5ccc6c(c5)sc5ccccc56)n4)ccc3o2)cc1]

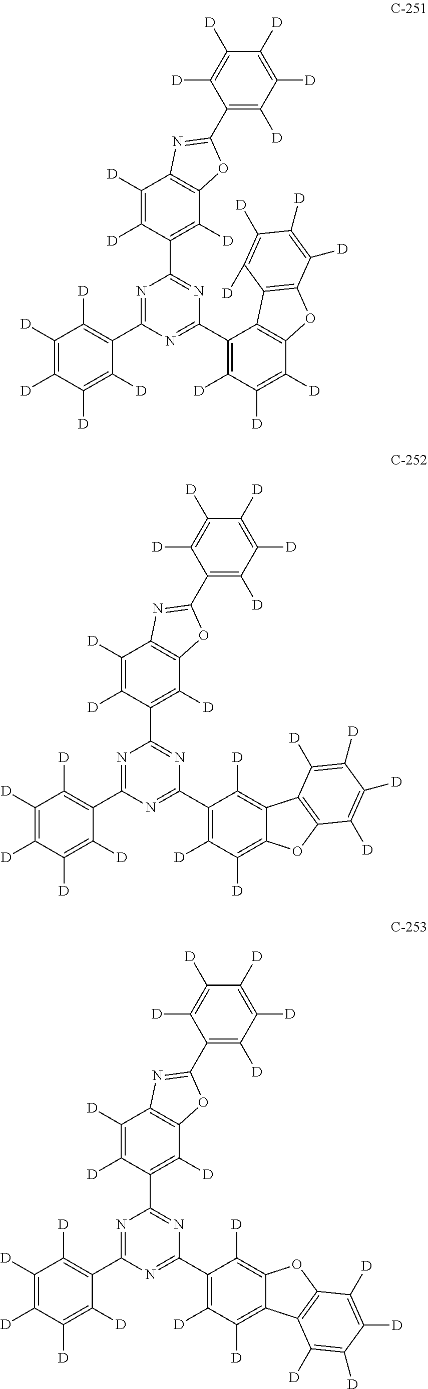

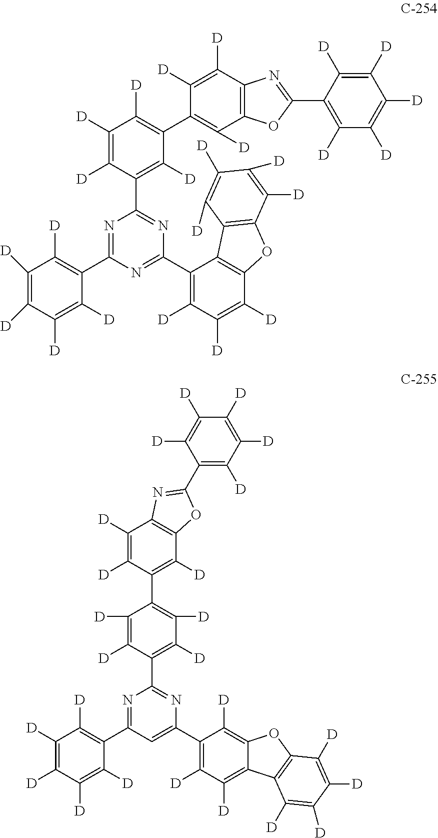 [SMILES: [2H]c1c([2H])c([2H])c(-c2nc(-c3c([2H])c([2H])c([2H])c(-c4c([2H])c([2H])c5nc(-c6c([2H])c([2H])c([2H])c([2H])c6[2H])oc5c4[2H])c3[2H])nc(-c3c([2H])c([2H])c([2H])c4oc5c([2H])c([2H])c([2H])c([2H])c5c34)n2)c([2H])c1[2H] ; [2H]c1c([2H])c([2H])c(-c2cc(-c3c([2H])c([2H])c4c(oc5c([2H])c([2H])c([2H])c([2H])c54)c3[2H])nc(-c3c([2H])c([2H])c(-c4c([2H])c([2H])c5nc(-c6c([2H])c([2H])c([2H])c([2H])c6[2H])oc5c4[2H])c([2H])c3[2H])n2)c([2H])c1[2H]]

The compound represented by formula 1 according to the present disclosure may be prepared by a synthetic method known to one skilled in the art. For example, the compound represented by formula 1 may be prepared by referring to the following reaction schemes 1-1 and 1-2, and the compound represented by formula 3 according to the present disclosure may be prepared by referring to the following reaction scheme 1-1, but is not limited thereto.

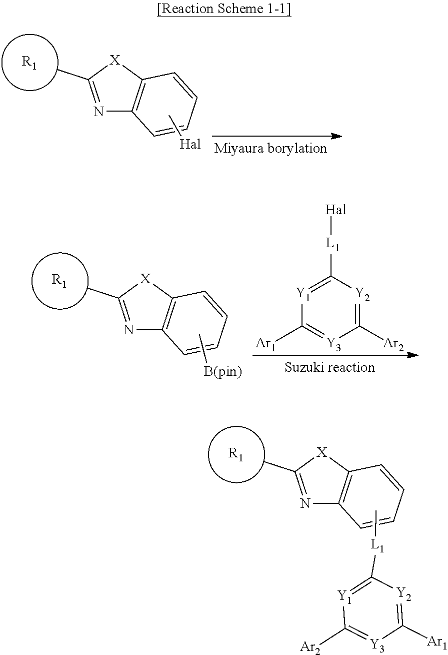

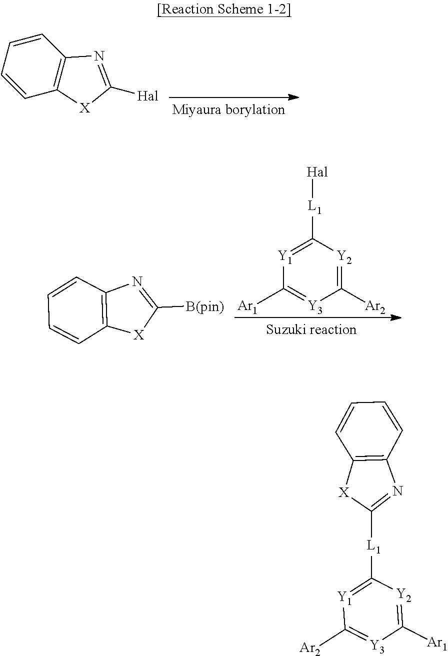

In reaction schemes 1-1 and 1-2, X, R₁, Y₁ to Y₃, L₁, Ar₁, and Ar₂ are as defined in formula 1, and Hal means a halogen.

The compound represented by formula 2 according to the present disclosure may be prepared by a synthetic method known to one skilled in the art. For example, the compound represented by formula 2 may be prepared by referring to the following reaction scheme 2, but is not limited thereto.

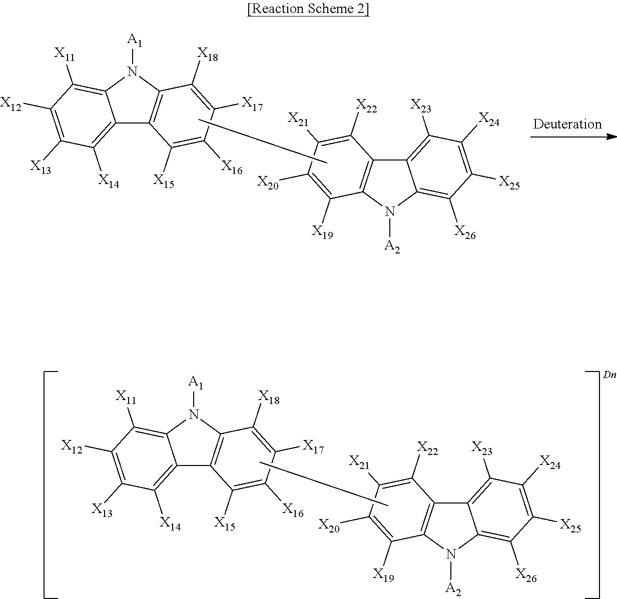

In reaction scheme 2, A₁, A₂, and X₁₁ to X₂₆ are as defined in formula 2, Dn means that n number of hydrogens have been replaced by deuterium, n is an integer of 1 or more, and the upper limit of n is the number of hydrogens in each compound.

Although illustrative synthesis examples of the compounds represented by formulas 1 to 3 are described above, one skilled in the art will be able to readily understand that all of them are based on a Buchwald-Hartwig cross coupling reaction, an N-arylation reaction, a H-mont-mediated etherification reaction, a Miyaura borylation reaction, a Suzuki cross-coupling reaction, an Intramolecular acid-induced cyclization reaction, a Pd(II)-catalyzed oxidative cyclization reaction, a Grignard reaction, a Heck reaction, a Cyclic Dehydration reaction, an SN1 substitution reaction, an SN2 substitution reaction, and a Phosphine-mediated reductive cyclization reaction, etc., and the above reactions proceed even when substituents defined in formulas 1 to 3 other than the substituents specified in the specific synthesis examples, are bonded.

In addition, the deuterated compounds of formulas 1 to 3 can be prepared in a similar manner using deuterated precursor materials, or more commonly, can be prepared by treating non-deuterated compounds with a deuterated solvent, D6-benzene, in the presence of a Lewis acid H/D exchange catalyst such as aluminum trichloride or ethyl aluminum chloride. Also, the degree of deuteration can be controlled by varying reaction conditions such as reaction temperature. For example, the number of deuterium in the formulas 1 to 3 can be controlled by adjusting the reaction temperature and time, the equivalent of acid, etc.

The present disclosure provides an organic electroluminescent compound represented by formula 3, an organic electroluminescent material comprising the organic electroluminescent compound represented by formula 3, and an organic electroluminescent device comprising the organic electroluminescent material. The organic electroluminescent material may consist of the organic electroluminescent compound of the present disclosure alone, and may further include conventional materials included in the organic electroluminescent material.

The organic electroluminescent device of the present disclosure comprises an anode, a cathode, and at least one organic layer between the anode and the cathode, wherein the organic layer may comprise an organic electroluminescent material including the compound represented by formula 3.

The organic electroluminescent compound of formula 3 of the present disclosure may be comprised in at least one layer of a light-emitting layer, a hole injection layer, a hole transport layer, a hole auxiliary layer, a light-emitting auxiliary layer, an electron transport layer, an electron buffer layer, an electron injection layer, an interlayer, a hole blocking layer, and an electron blocking layer, and may preferably be comprised in at least one layer of a light-emitting layer, a hole transport layer, a hole auxiliary layer, a light-emitting auxiliary layer, an electron transport layer, an electron buffer layer, a hole blocking layer, and an electron blocking layer, if necessary. The organic electroluminescent compound of formula 3 of the present disclosure may be comprised as a host material. If necessary, the organic electroluminescent compound of the present disclosure may be used as a co-host material.

The present disclosure provides an organic electroluminescent device comprising an anode; a cathode; and at least one light-emitting layer between the anode and the cathode, wherein the at least one light-emitting layer comprises a plurality of host materials of the present disclosure. The first host material and the second host material of the present disclosure may be comprised in one light-emitting layer or may be respectively comprised in different light-emitting layers among a plurality of light-emitting layers. The plurality of host materials of the present disclosure may comprise the compound represented by formula 1 and the compound represented by formula 2 at a ratio of about 1:99 to about 99:1, preferably about 10:90 to about 90:10, and more preferably about 30:70 to about 70:30. In addition, the compound represented by formula 1 and the compound represented by formula 2 in a desired ratio may be combined by mixing them in a shaker, by dissolving them in a glass tube by heat, or by dissolving them in a solvent, etc.

According to one embodiment of the present disclosure, the doping concentration of the dopant compound with respect to the host compound of the light-emitting layer may be less than 20 wt %. The dopants comprised in the organic electroluminescent device of the present disclosure may be at least one phosphorescent or fluorescent dopant, and is preferably a phosphorescent dopant. The phosphorescent dopant materials applied to the organic electroluminescent device according to the present disclosure are not particularly limited, but may be selected from metallated complex compounds of iridium (Ir), osmium (Os), copper (Cu), and platinum (Pt), preferably selected from ortho-metallated complex compounds of iridium (Ir), osmium (Os), copper (Cu), and platinum (Pt), and more preferably an ortho-metallated iridium complex compound.

The dopant comprised in the organic electroluminescent device of the present disclosure may be a compound represented by the following formula 101, but is not limited thereto.

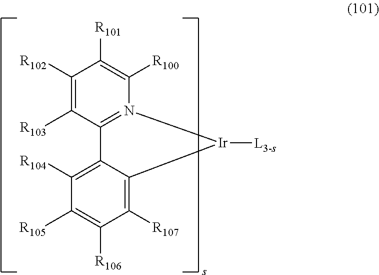

In formula 101,
L is any one selected from the following structures 1 to 3;

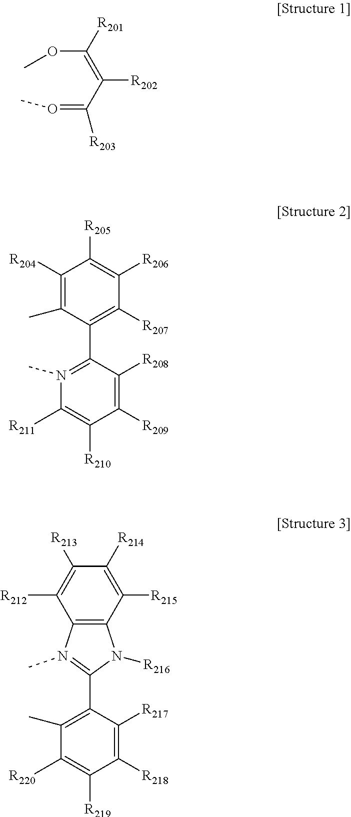

R₁₀₀ to R₁₀₃, each independently, represent hydrogen, deuterium, a halogen, a (C1-C30)alkyl unsubstituted or substituted with deuterium and/or a halogen(s), a substituted or unsubstituted (C3-C30)cycloalkyl, a substituted or unsubstituted (C6-C30)aryl, a cyano, a substituted or unsubstituted (3- to 30-membered)heteroaryl, or a substituted or unsubstituted (C1-C30)alkoxy; or may be linked to an adjacent substituent to form a ring(s), e.g., a substituted or unsubstituted, quinoline, benzofuropyridine, benzothienopyridine, indenopyridine, benzofuroquinoline, benzothienoquinoline, or indenoquinoline, together with pyridine;
R₁₀₄ to R₁₀₇, each independently, represent hydrogen, deuterium, a halogen, a (C1-C30)alkyl unsubstituted or substituted with deuterium and/or a halogen(s), a substituted or unsubstituted (C3-C30)cycloalkyl, a substituted or unsubstituted (C6-C30)aryl, a substituted or unsubstituted (3- to 30-membered)heteroaryl, a cyano, or a substituted or unsubstituted (C1-C30)alkoxy; or may be linked to an adjacent substituent to form a substituted or unsubstituted ring(s), e.g., a substituted or unsubstituted, naphthalene, fluorene, dibenzothiophene, dibenzofuran, indenopyridine, benzofuropyridine, or benzothienopyridine, together with benzene;
R₂₀₁ to R₂₂₀, each independently, represent hydrogen, deuterium, a halogen, a (C1-C30)alkyl unsubstituted or substituted with deuterium and/or a halogen(s), a substituted or unsubstituted (C3-C30)cycloalkyl, or a substituted or unsubstituted (C6-C30)aryl; or may be linked to an adjacent substituent to form a substituted or unsubstituted ring(s); and
s represents an integer of 1 to 3.

The specific examples of the dopant compound are as follows, but are not limited thereto.

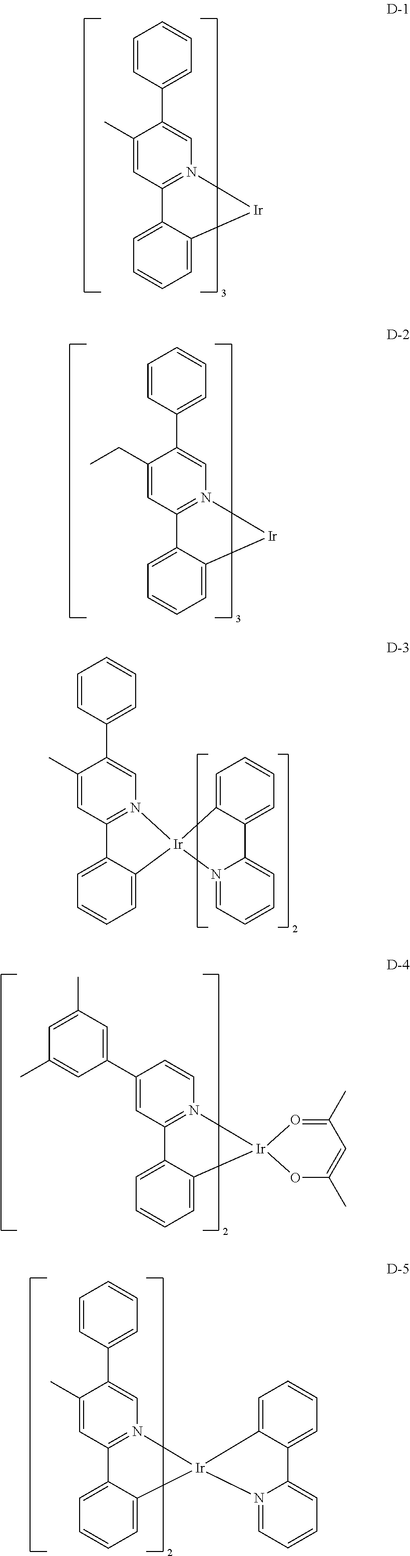

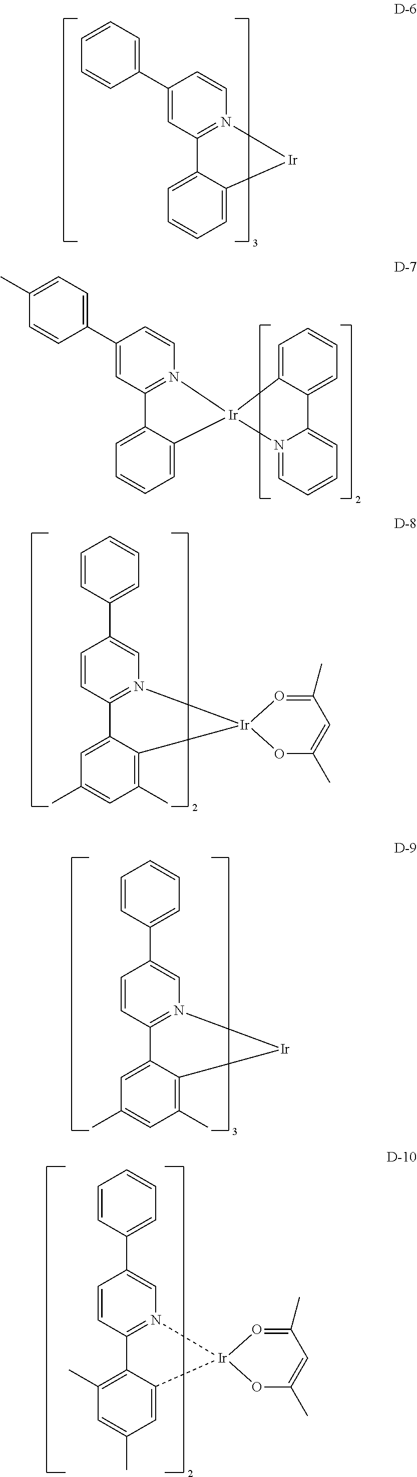

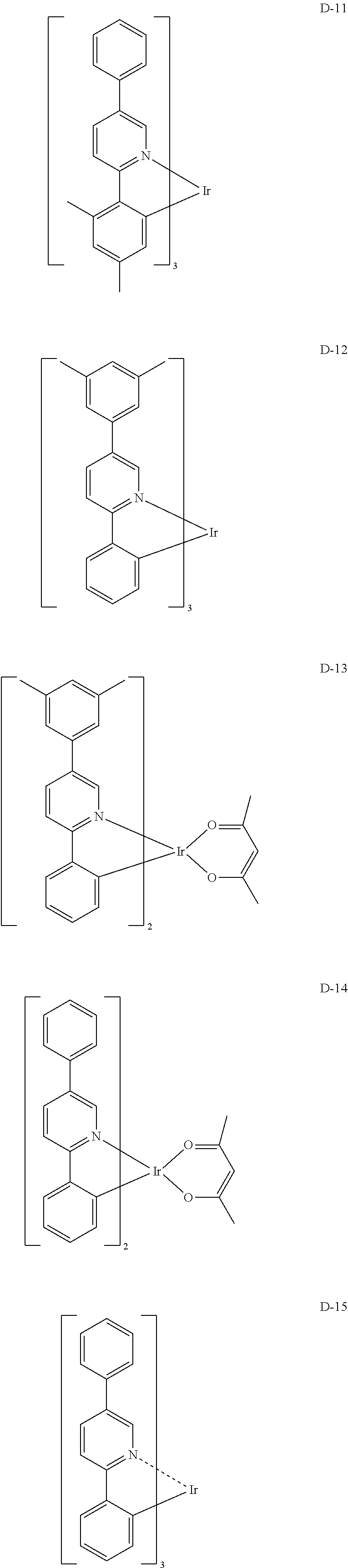

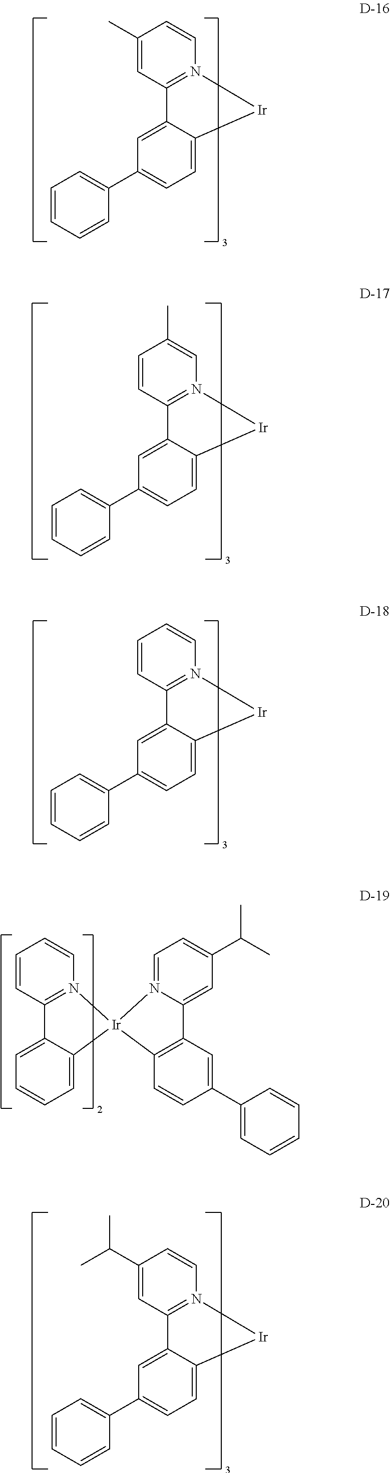

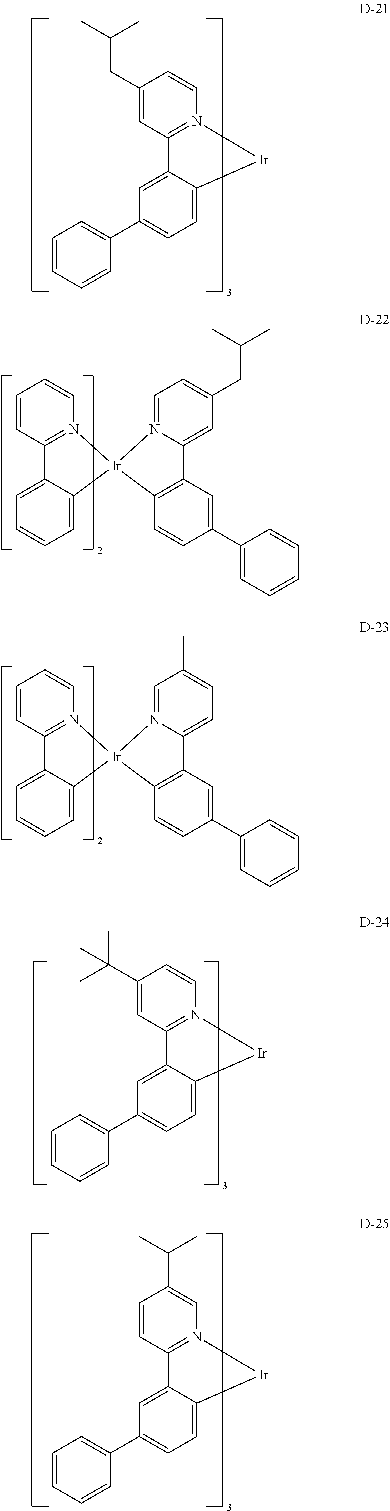

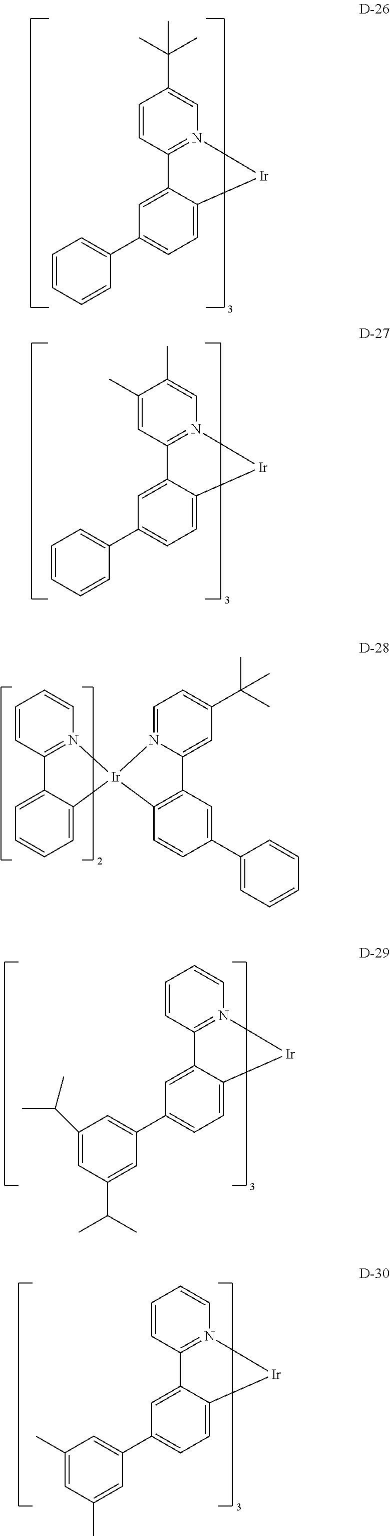

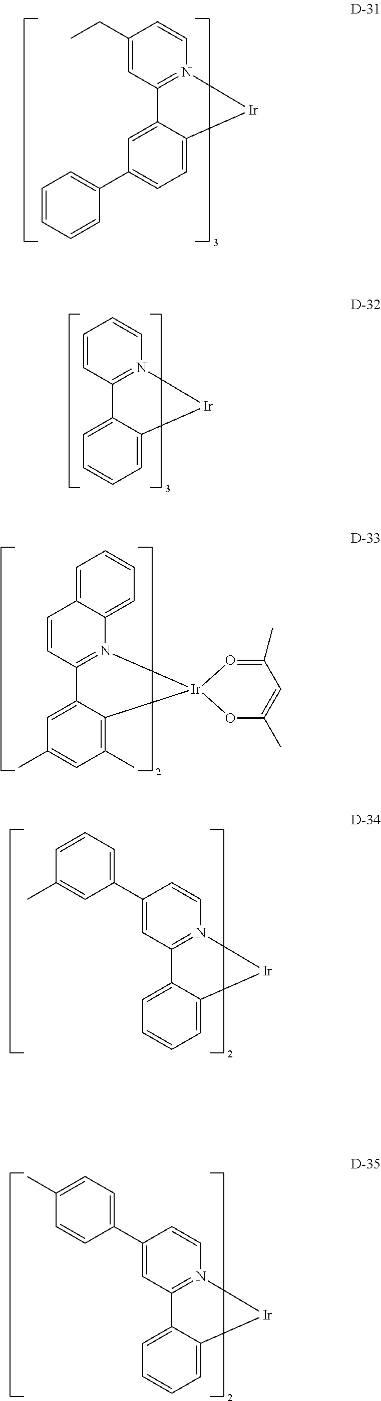

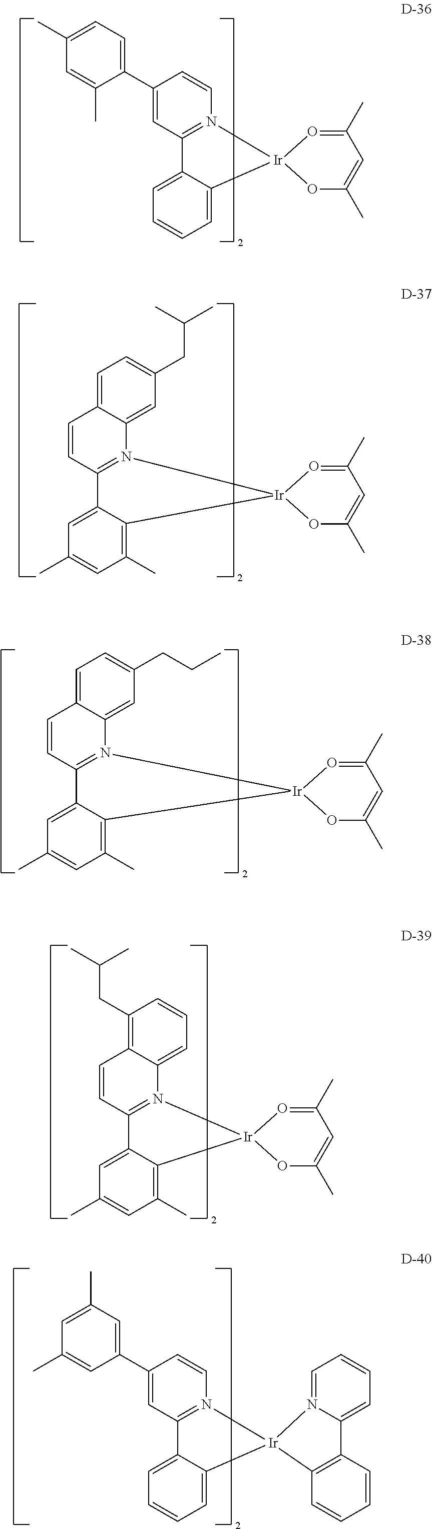

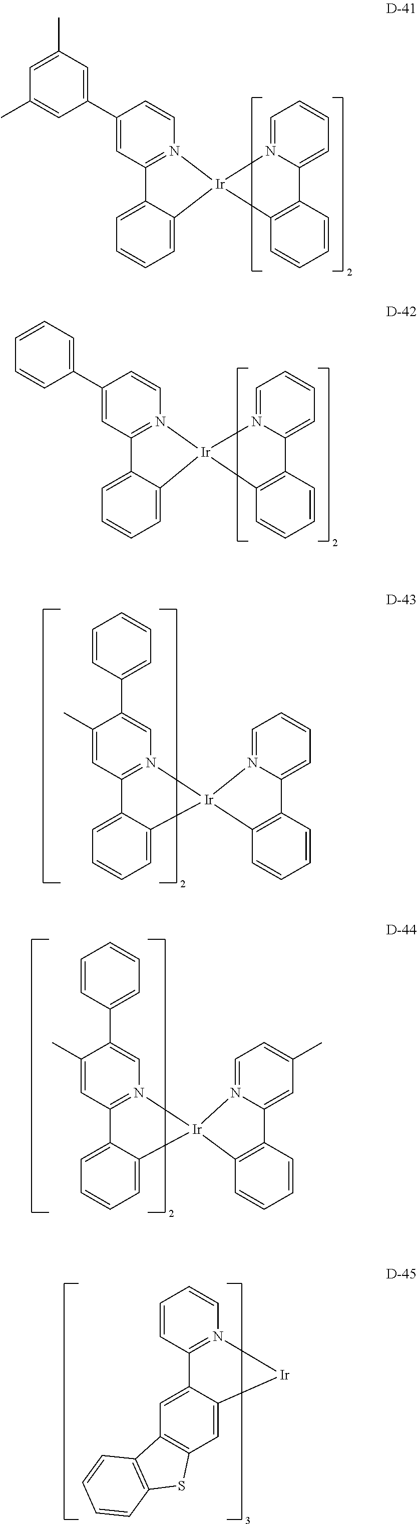

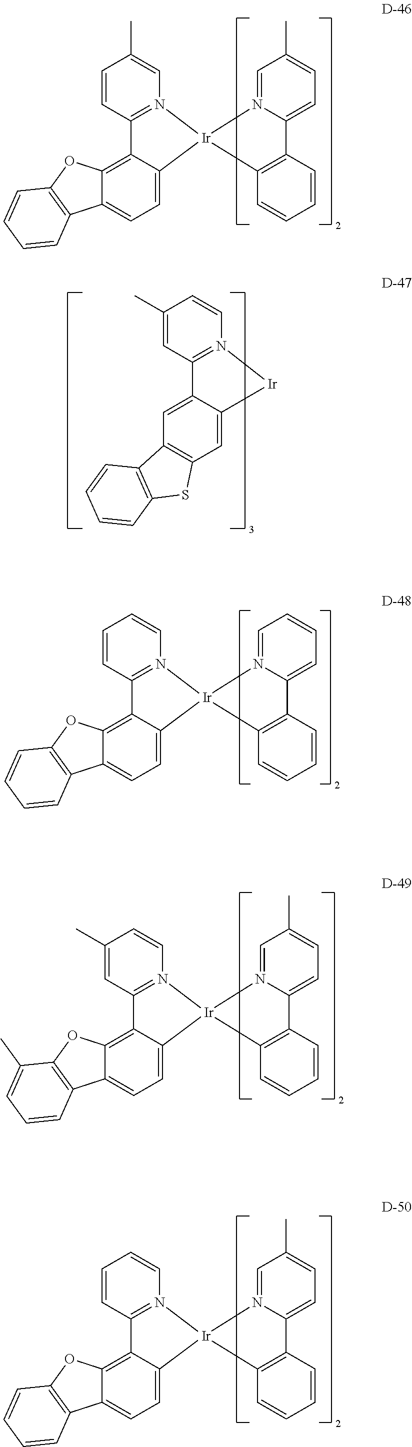

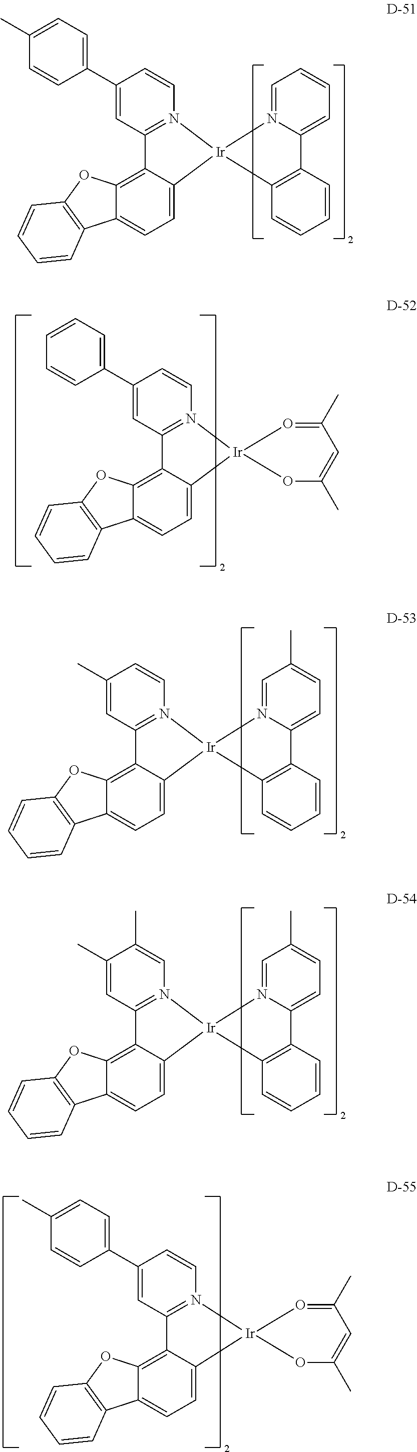

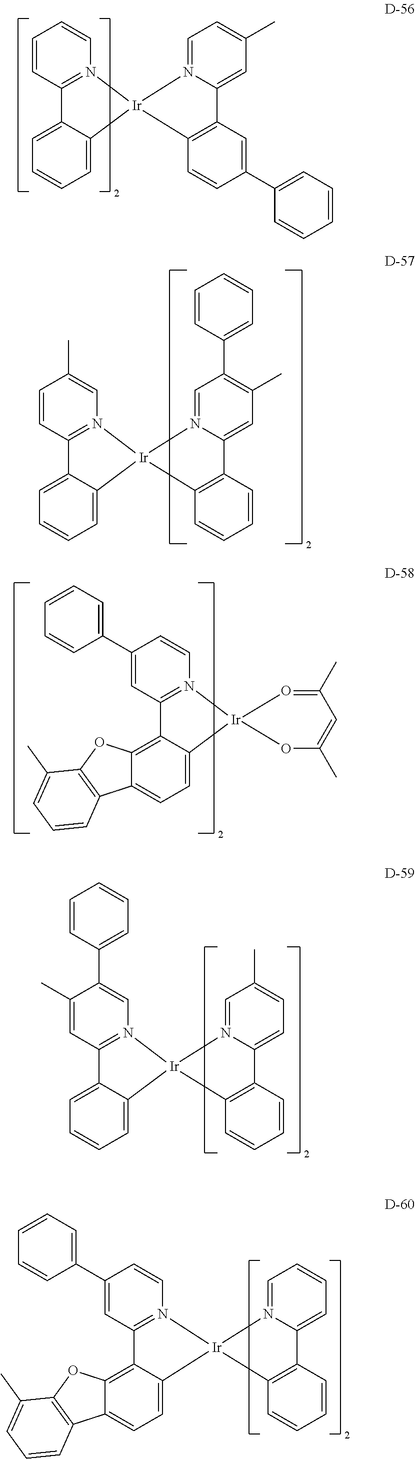

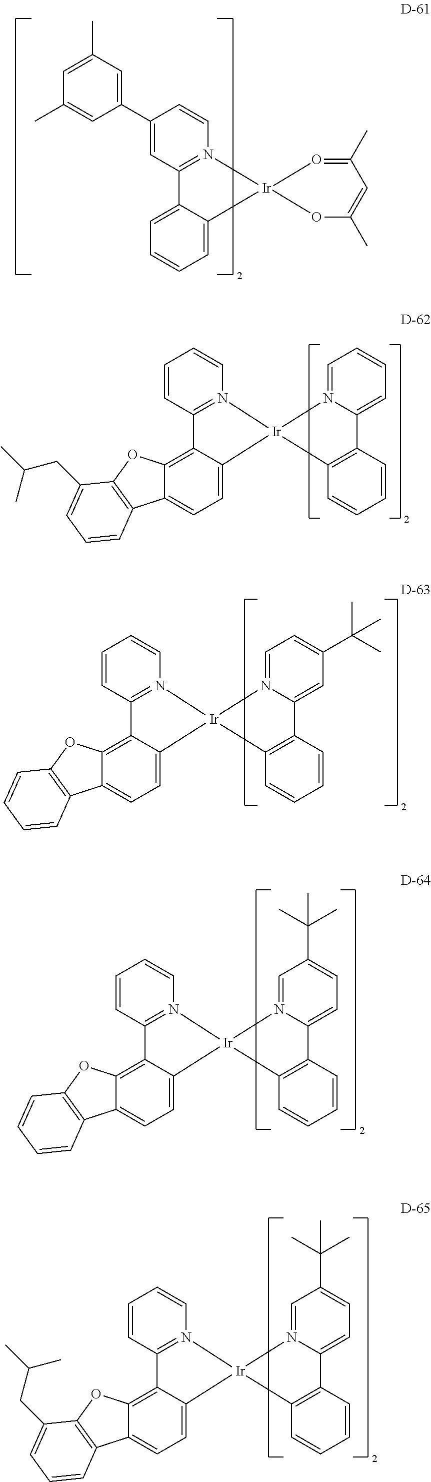

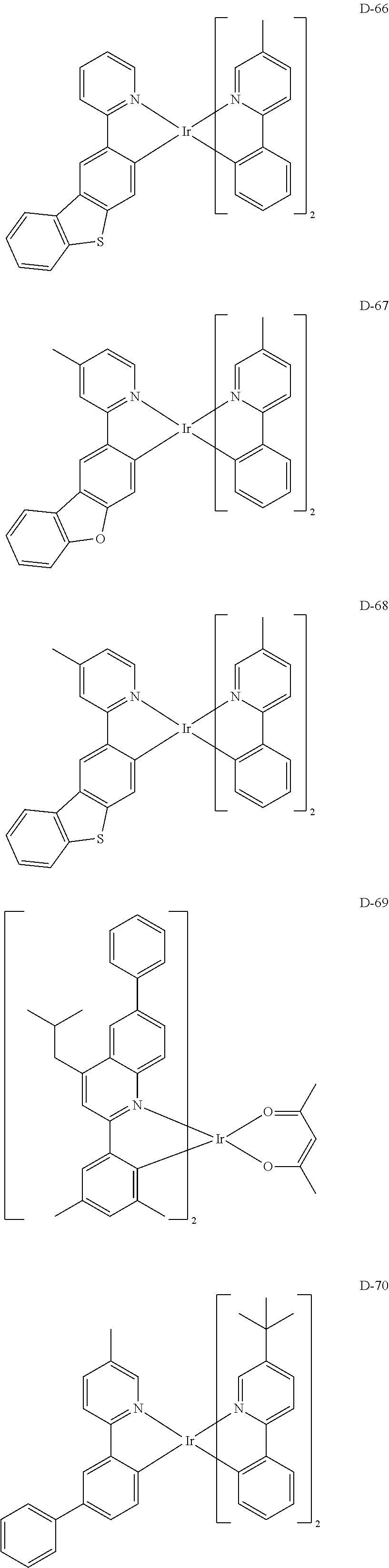

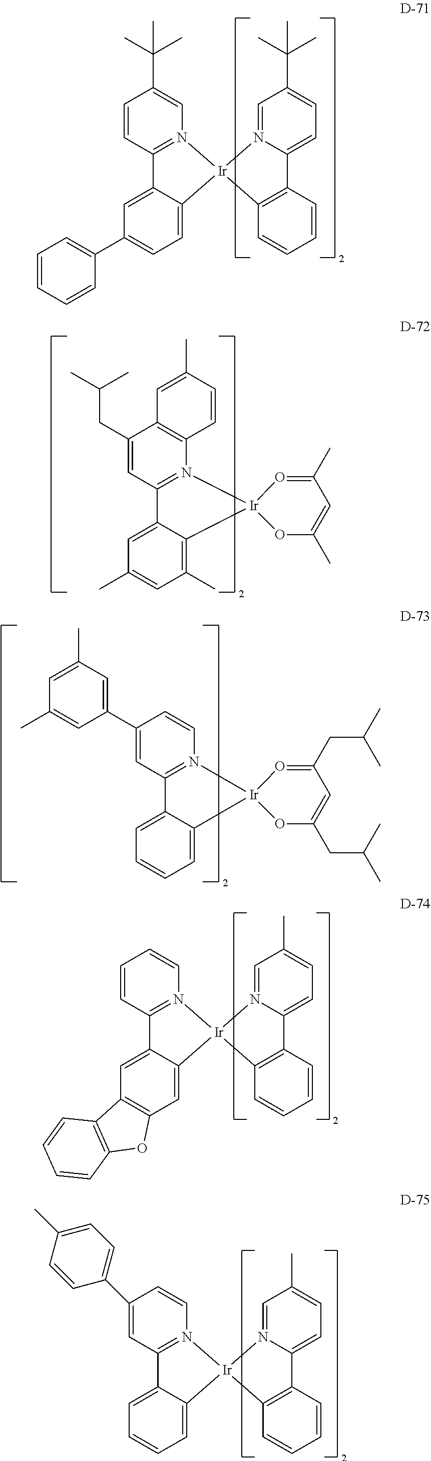

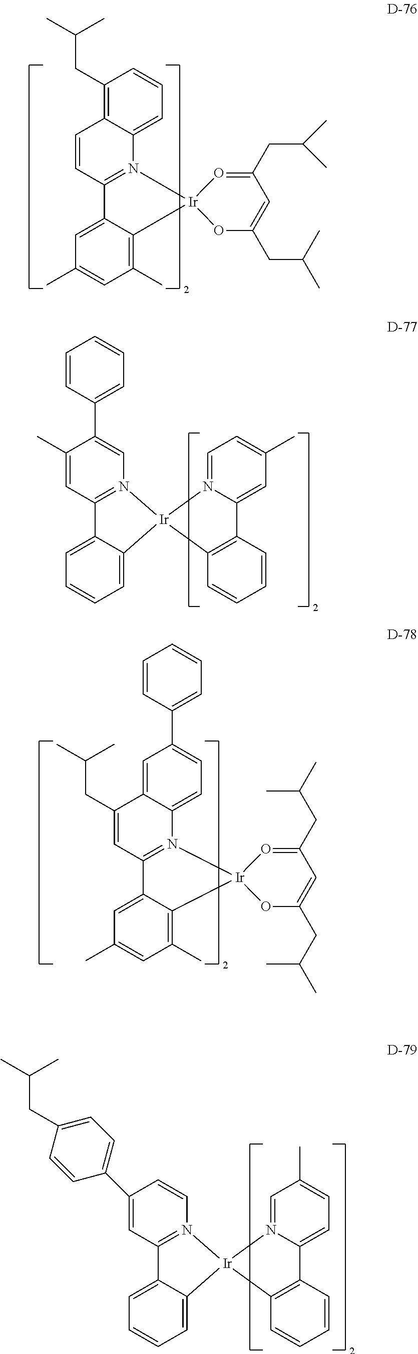

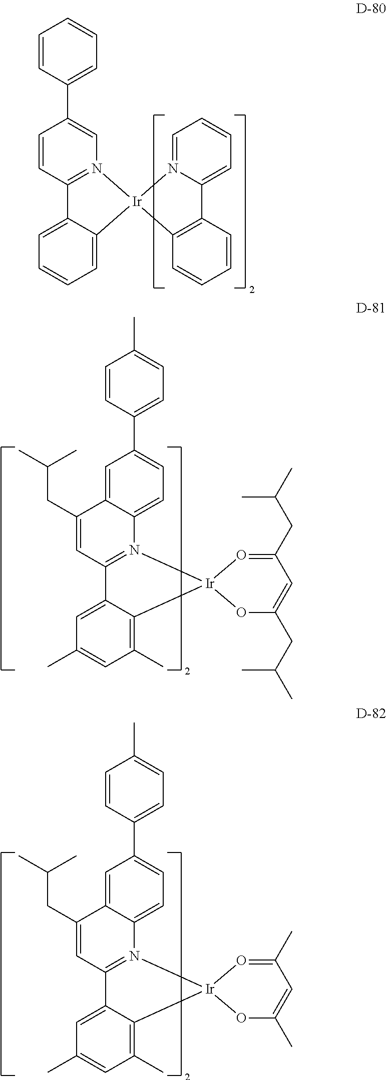

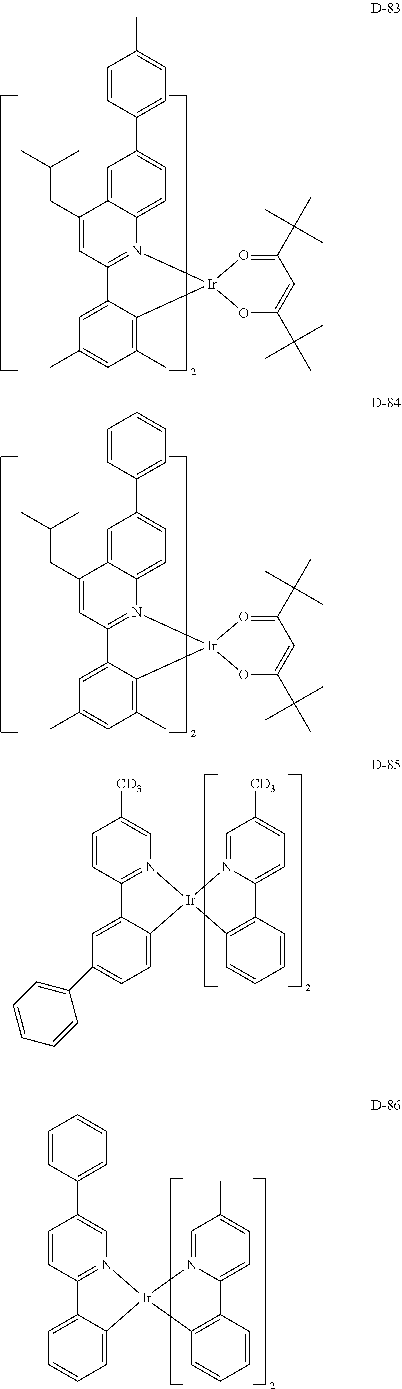

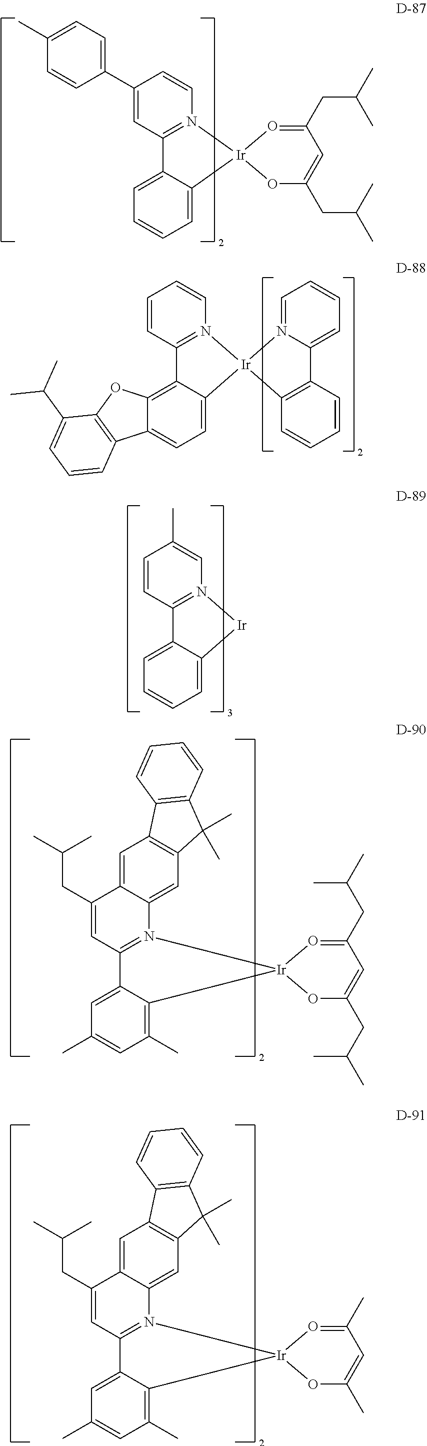

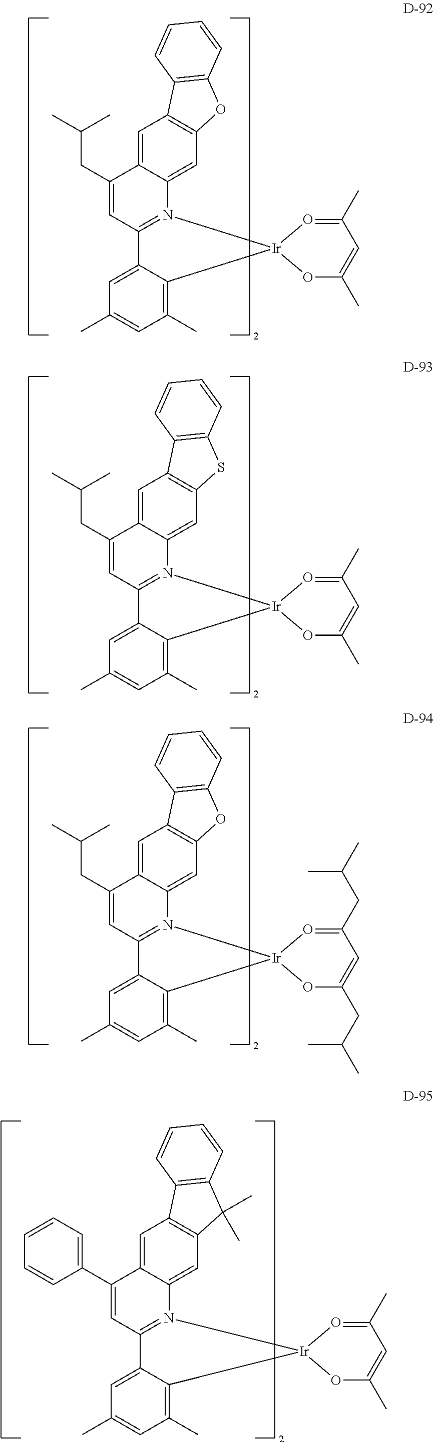

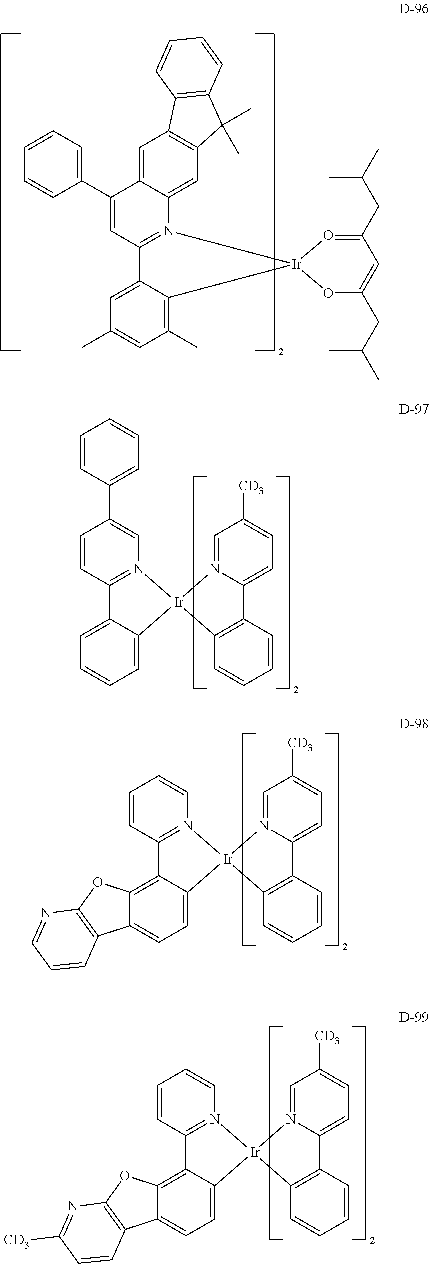

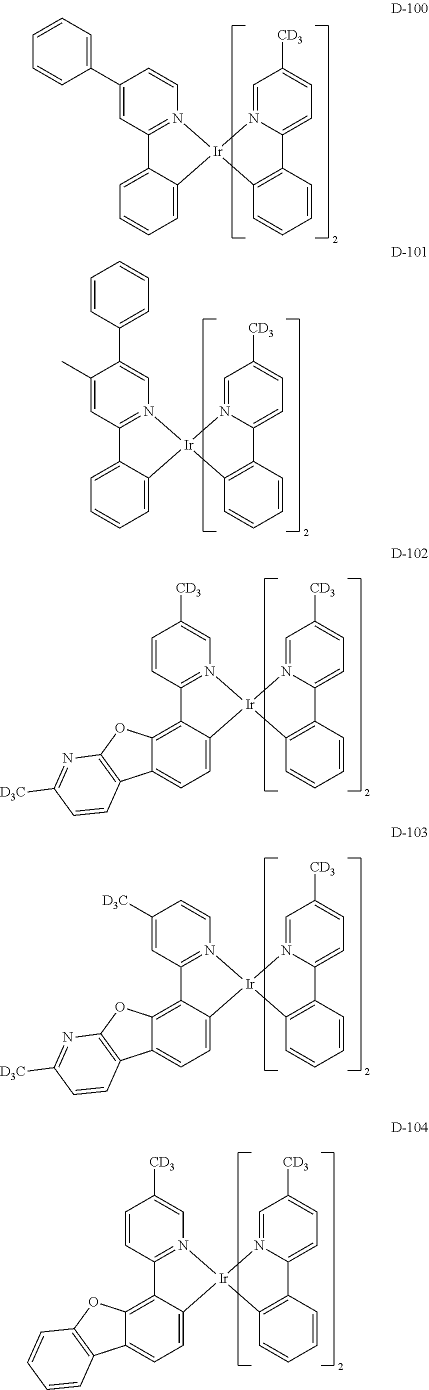

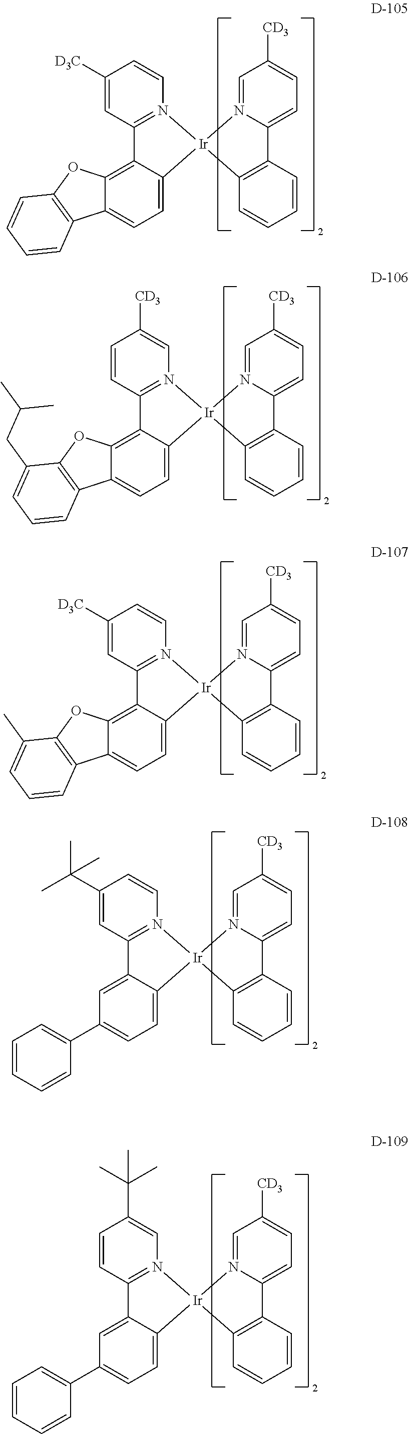

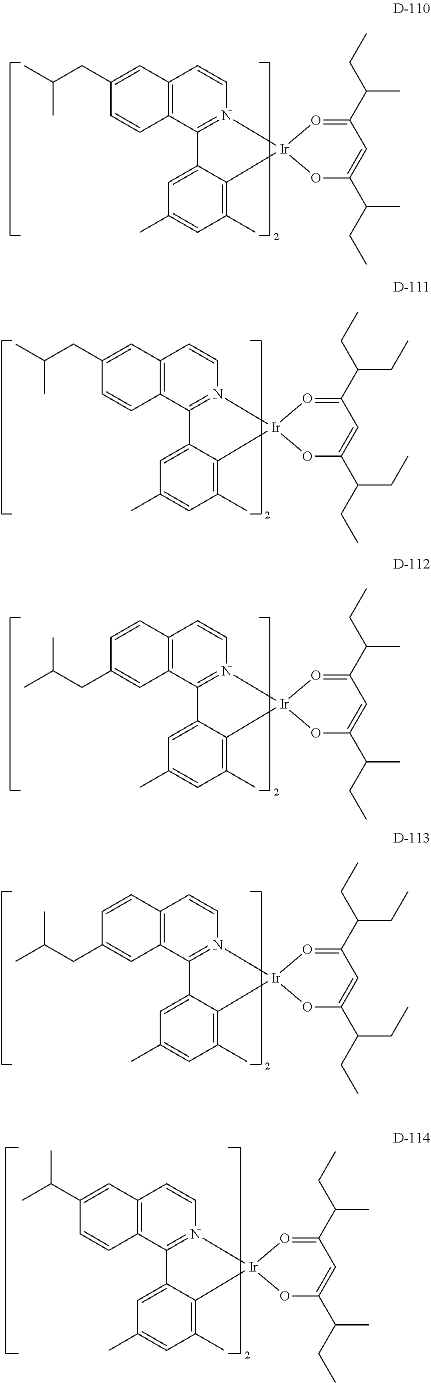

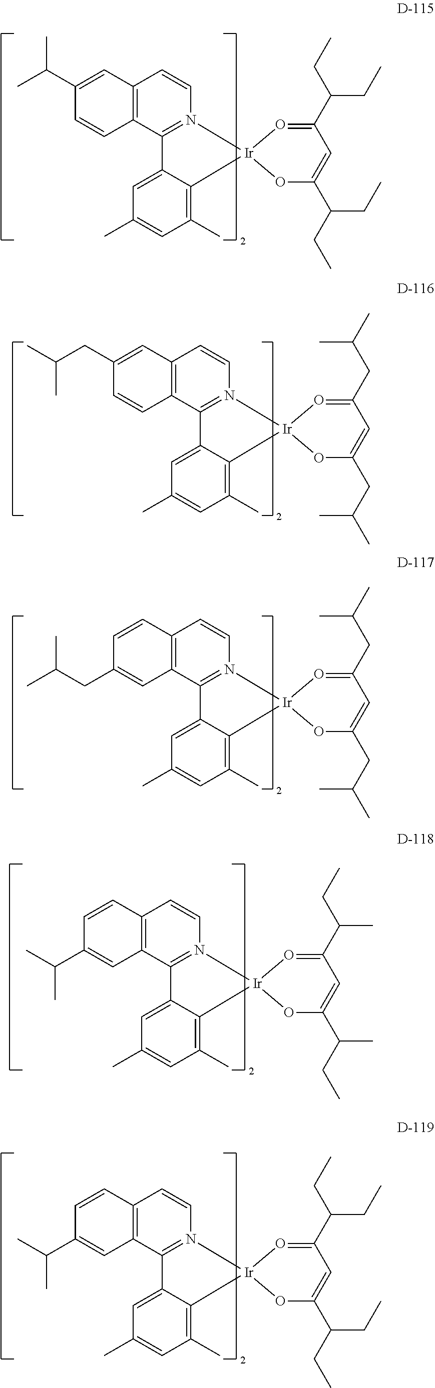

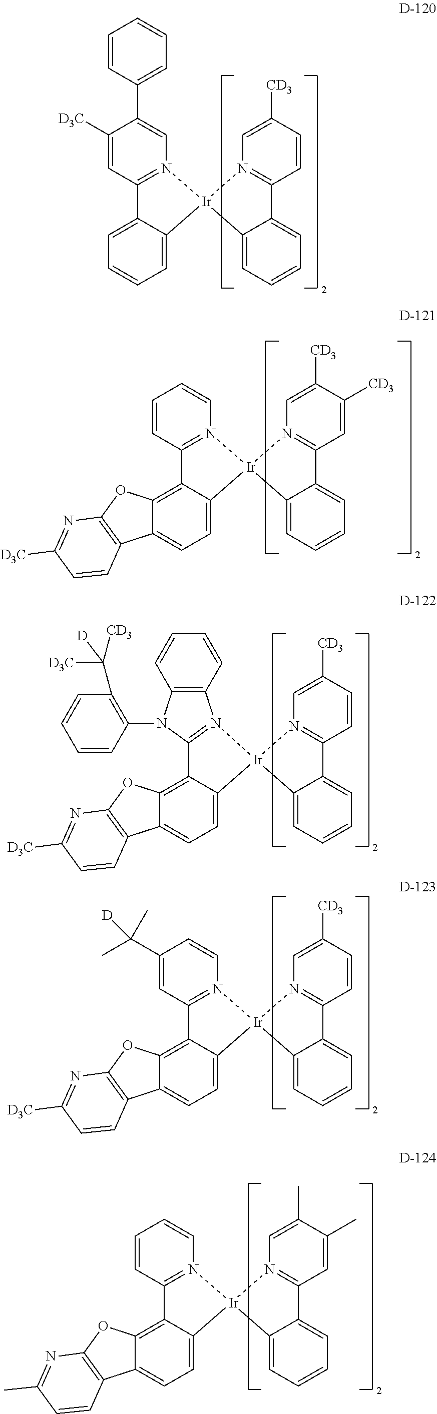

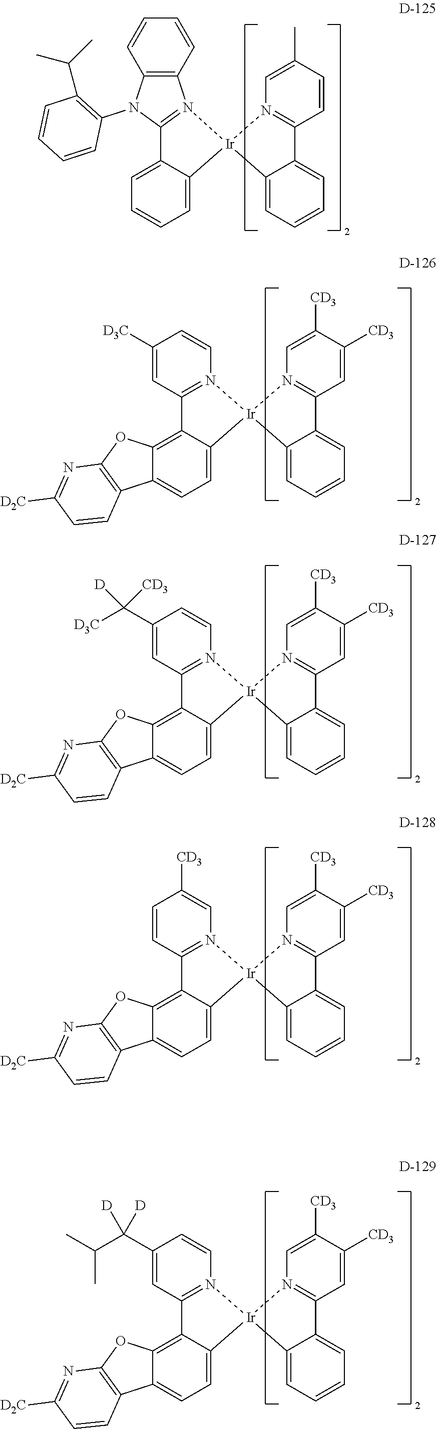

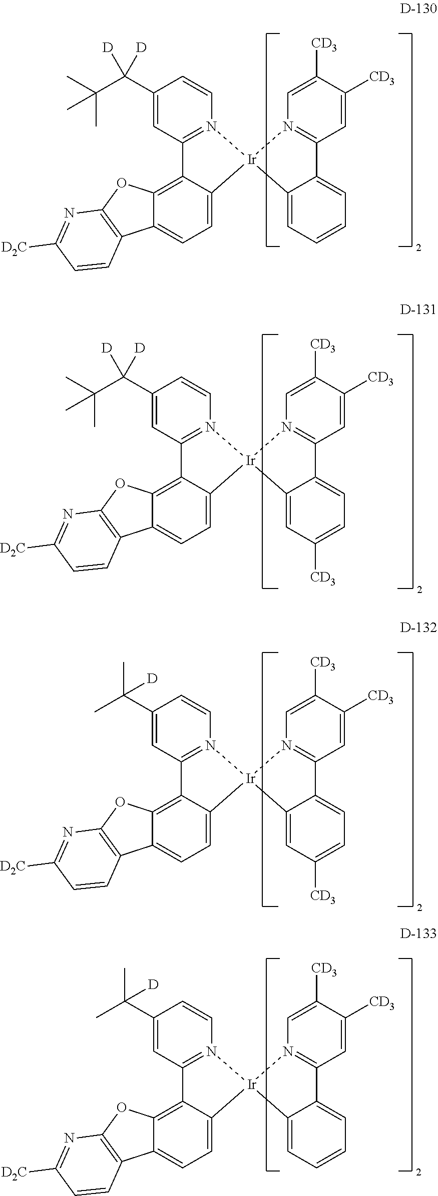

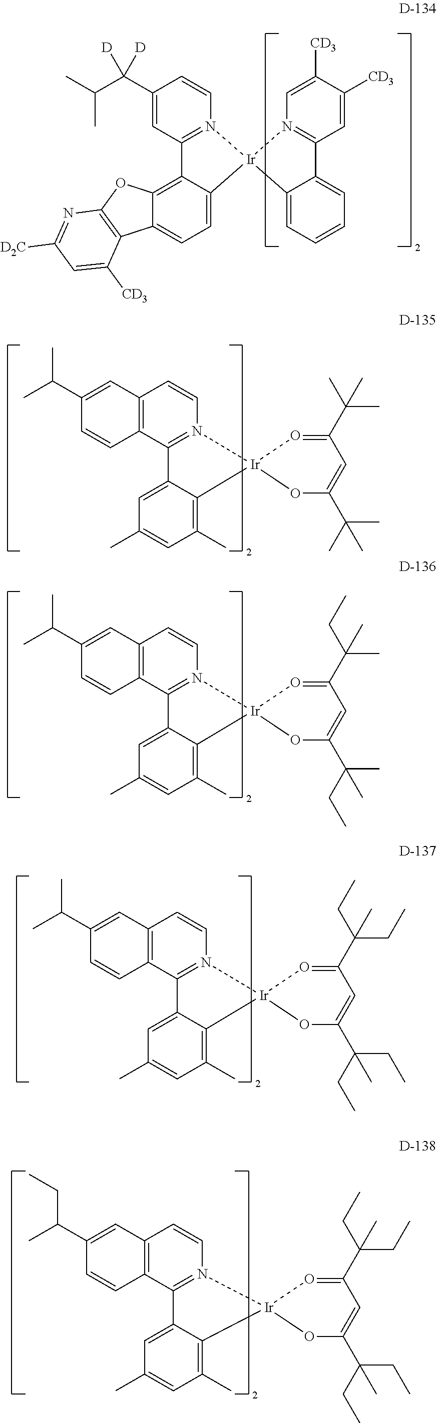

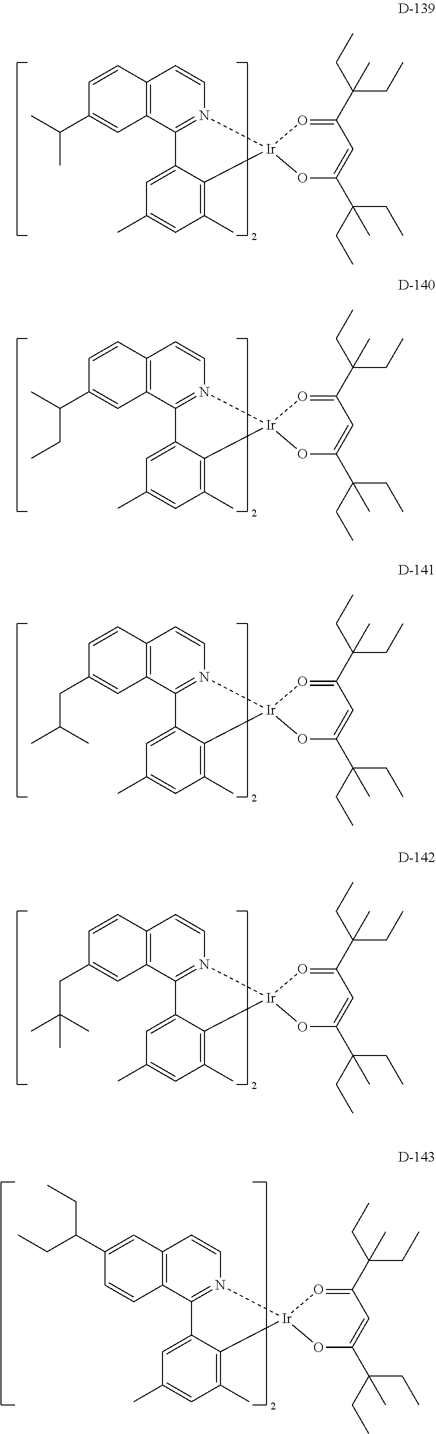

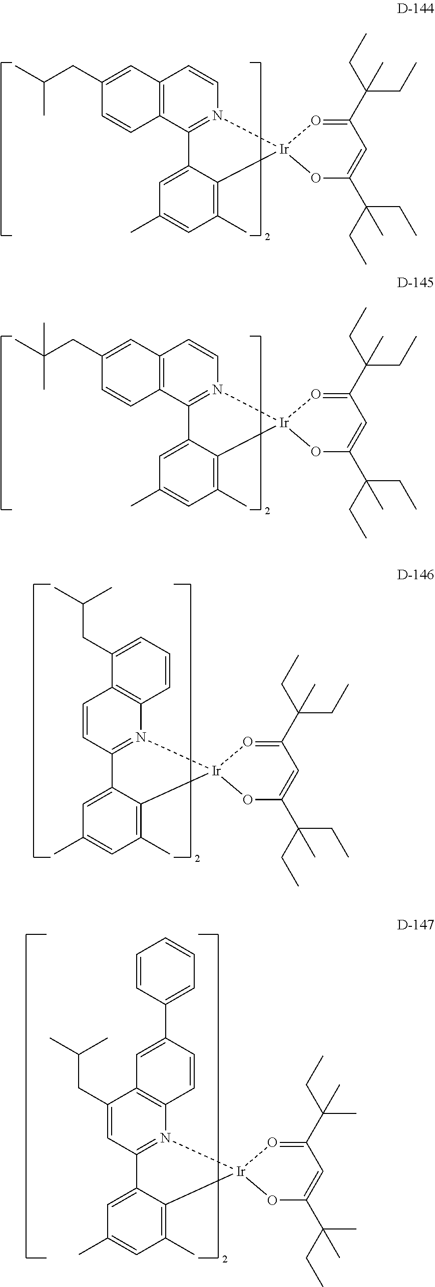

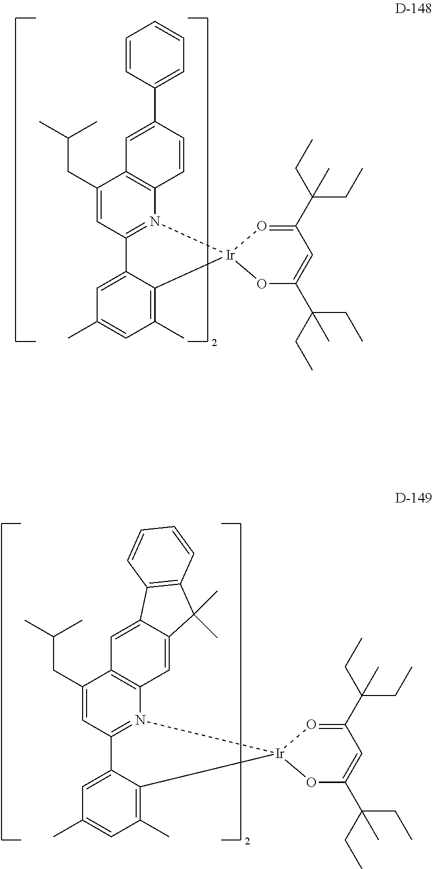

The organic electroluminescent device of the present disclosure comprises an anode; a cathode; and at least one organic layer between the anode and the cathode. The organic layer comprises a light-emitting layer, and may further comprise at least one layer selected from a hole injection layer, a hole transport layer, a hole auxiliary layer, an light-emitting auxiliary layer, an electron transport layer, an electron buffer layer, an electron injection layer, an interlayer, a hole blocking layer, and an electron blocking layer. Each of the layers may be further configured as a plurality of layers.

Each of the anode and the cathode may be formed of a transparent conductive material or a transflective or reflective conductive material. Depending on the type of material forming the anode and the cathode, the organic electroluminescent device may be a top light-emitting type, a bottom light-emitting type, or a double side light-emitting type. In addition, the hole injection layer may be further doped with a p-dopant(s), and the electron injection layer may be further doped with an n-dopant(s).

At least one compounds selected from the group consisting of arylamine-based compounds and styrylarylamine-based compounds may be further comprised in the organic layer. In addition, the organic material layer may further comprise at least one metal selected from the group consisting of metals of Group 1, metals of Group 2, transition metals of the 4^{th} period, transition metals of the 5^{th} period, lanthanides, and organic metals of the d-transition elements of Periodic Table, or at least one complex compound comprising the metal.

In addition, the organic electroluminescent device of the present disclosure may emit white light by further comprising at least one light-emitting layer, which comprises a blue, a red or a green light-emitting compound known in the art, besides the compound of the present disclosure. In addition, if necessary, it may further comprise a yellow or an orange light-emitting layer.

In the organic electroluminescent device of the present disclosure, it is preferable to dispose at least one layer selected from a chalcogenide layer, a metal halide layer, and a metal oxide layer (hereinafter referred to as “surface layer”) on at least one inner surface of a pair of electrodes. Specifically, a chalcogenide (including oxide) layer of silicon and aluminum is preferably placed on an anode surface of an electroluminescent medium layer side, and a metal halide layer or a metal oxide layer is preferably placed on a cathode surface of an electroluminescent medium layer side. Driving stabilization of the organic electroluminescent device can be obtained by the surface layer. Preferred examples of the chalcogenide include SiO_{X}(1≤X≤2), AlO_{X}(1≤X≤1.5), SiON, SiAlON, etc., preferred examples of the metal halide include LiF, MgF₂, CaF₂, a rare earth metal fluoride, etc, and preferred examples of the metal oxide include Cs₂O, Li₂O, MgO, SrO, BaO, CaO, etc.

A hole injection layer, a hole transport layer or an electron blocking layer, or a combination thereof may be used between an anode and a light-emitting layer. The hole injection layer may be multi-layers in order to lower the hole injection barrier (or hole injection voltage) from the anode to the hole transport layer or the electron blocking layer, wherein two compounds may be simultaneously used in each of the multi-layers. The hole transport layer or the electron blocking layer may be multi-layers.

An electron buffer layer, a hole blocking layer, an electron transport layer or an electron injection layer, or a combination thereof may be used between a light-emitting layer and a cathode. The electron buffer layer may be multi-layers in order to control electron injection and improve interfacial properties between the light-emitting layer and the electron injection layer, wherein two compounds may be simultaneously used in each of the multi-layers. The hole blocking layer or the electron transport layer may be multi-layers, wherein a plurality of compounds may be used in each of the multi-layers.

A light-emitting auxiliary layer may be a layer placed between an anode and a light-emitting layer, or between a cathode and a light-emitting layer. When placed between the anode and the light-emitting layer, the light-emitting auxiliary layer may be used to facilitate hole injection and/or hole transport or to block the overflow of electrons. When placed between the cathode and the light-emitting layer, the light-emitting auxiliary layer may be used to facilitate electron injection and/or electron transport or to block the overflow of holes. In addition, the hole auxiliary layer may be placed between the hole transport layer (or hole injection layer) and the light-emitting layer, and may exhibit an effect of facilitating or blocking the hole transport rate (or hole injection rate), and accordingly, may adjust the charge balance. In addition, the electron blocking layer may be placed between the hole transport layer (or hole injection layer) and the light-emitting layer, and may block the overflow of electrons from the light-emitting layer and confine the excitons in the light-emitting layer to prevent light leakage. When an organic electroluminescent device includes two or more hole transport layers, the hole transport layer, which is further included, may be used as a hole auxiliary layer or an electron blocking layer. The light-emitting auxiliary layer, the hole auxiliary layer, or the electron blocking layer may have an effect of improving the efficiency and/or lifetime of the organic electroluminescent device.

In addition, in an organic electroluminescent device of the present disclosure, a mixed region of an electron transport compound and a reductive dopant, or a mixed region of a hole transport compound and an oxidative dopant may be placed on at least one surface of a pair of electrodes. In this case, the electron transport compound is reduced to an anion, and thus it becomes easier to inject and transport electrons from the mixed region to the light-emitting medium. Furthermore, the hole transport compound is oxidized to a cation, and thus it becomes easier to inject and transport holes from the mixed region to the light-emitting medium. Preferred oxidative dopants include various Lewis acids and acceptor compounds, and preferred reductive dopants include alkali metals, alkali metal compounds, alkaline earth metals, rare earth metals, and mixtures thereof. In addition, an organic electroluminescent device having at least two light-emitting layers and emitting white light may be manufactured by using the reductive dopant layer as a charge-generating layer.

The organic electroluminescent material according to one embodiment of the present disclosure may be used as a light-emitting material for a white organic light-emitting device. The white organic light-emitting device has been suggested to have various structures such as a side-by-side structure or a stacking structure depending on the arrangement of R (Red), G (Green) or YG (Yellowish Green), and B (Blue) light-emitting parts, or a color conversion material (CCM) method, etc. In addition, the organic electroluminescent material according to one embodiment of the present disclosure may also be used in an organic electroluminescent device comprising a quantum dot (QD).

In order to form each layer of the organic electroluminescent device of the present disclosure, dry film-forming methods such as vacuum evaporation, sputtering, plasma, ion plating methods, etc., or wet film-forming methods such as ink jet printing, nozzle printing, slot coating, spin coating, dip coating, flow coating methods, etc., can be used. When forming a film of the first host compound and the second host compound of the present disclosure, co-deposition or mixed deposition is performed.

When using a wet film-forming method, a thin film may be formed by dissolving or diffusing materials forming each layer into any suitable solvent such as ethanol, chloroform, tetrahydrofuran, dioxane, etc. The solvent may be any solvent where the materials forming each layer can be dissolved or diffused, and where there are no problems in film-formation capability.

In addition, it is possible to produce a display system, e.g., a display system for smartphones, tablets, notebooks, PCs, TVs, or cars, or a lighting system, e.g., an outdoor or indoor lighting system by using the organic electroluminescent device of the present disclosure.

Hereinafter, the preparation method of the compound of the present disclosure, and the properties thereof, and the driving voltage and the luminous efficiency of the organic electroluminescent device (OLED) comprising a plurality of host materials according to the present disclosure will be explained in detail with reference to the representative compounds of the present disclosure. However, the following examples only describe the properties of the compound according to the present disclosure and the OLED comprising the same, and the present disclosure is not limited to the following examples.

### Example 1: Preparation of Compound C-116

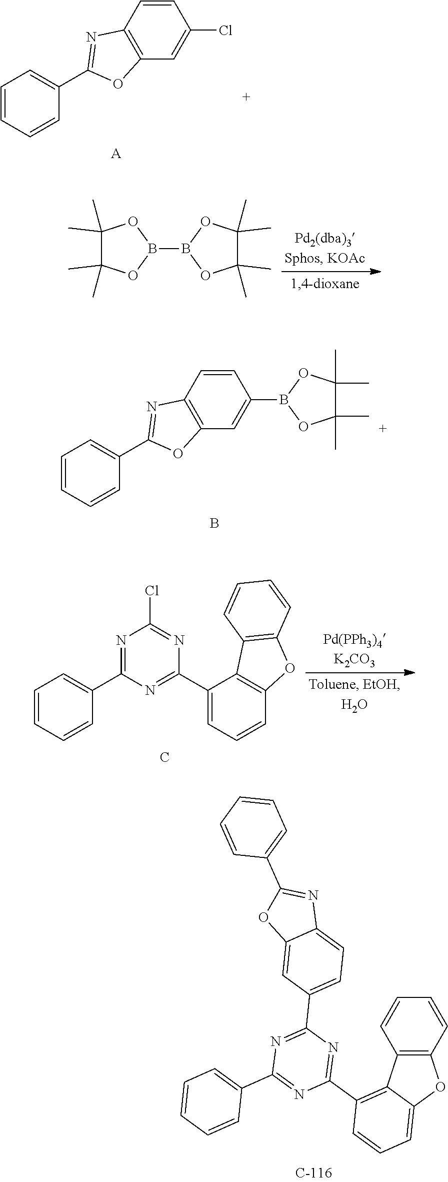

1) Synthesis of Compound B

Compound A (6 g, 26.1 mmol), bis(pinacolato)diboron (13.2 g, 52.2 mmol), Pd₂(dba)₃ (2.4 g, 2.6 mmol), Sphos (2.1 g, 5.2 mmol), and potassium acetate (7.7 g, 78.3 mmol) were added to a reaction vessel, dissolved in 130 mL of 1, 4-dioxane, and stirred under reflux for 3 hours. After the reaction was completed, the reaction product was washed with distilled water, and the organic layer was extracted with ethyl acetate, and dried by magnesium sulfate. The solvent was removed by rotary evaporator and the residue was purified by column chromatography to obtain Compound B (8 g, yield: 96%).

2) Synthesis of Compound C-116

Compound B (5 g, 15.6 mmol), Compound C (4.6 g, 12.9 mmol), Pd(PPh₃)₄ (750 mg, 0.65 mmol), and potassium carbonate (5.3 g, 38.9 mmol) were added to a reaction vessel, dissolved in 65 mL of toluene, 20 mL of ethanol, and 20 mL of water, and refluxed for 3 hours at 120° C. After the reaction was completed, the resulting solid was filtered, and washed with methanol. The filtrate was recrystallized by xylene and ethyl acetate to obtain Compound C-116 (2.6 g, yield: 32%).

| | MW | M.P. |
|---|---|---|
| C-116 | 516.56 | 273° C. |

### Example 2: Preparation of Compound C-117

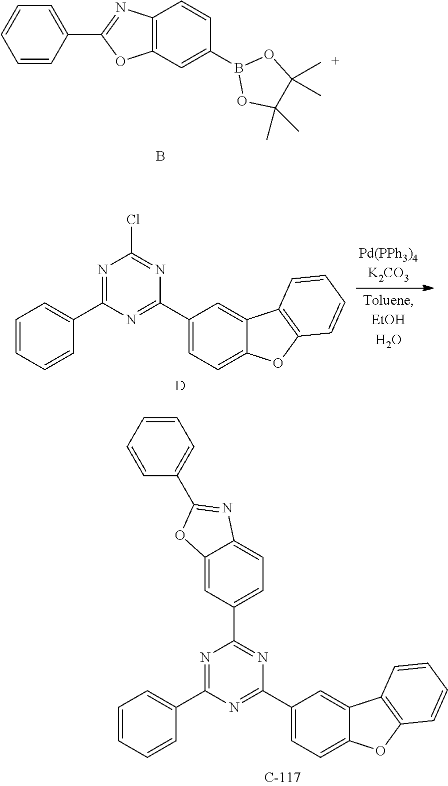

Compound B (6 g, 18.6 mmol), Compound D (6 g, 16.7 mmol), Pd(PPh₃)₄ (1.07 g, 0.92 mmol), and potassium carbonate (7.74 g, 56 mmol) were added to a reaction vessel, dissolved in 84 mL of toluene, 28 mL of ethanol, and 28 mL of water, and refluxed for 3 hours at 120° C. After the reaction was completed, the resulting solid was filtered, and washed with methanol. The filtrate was recrystallized by xylene and ethyl acetate to obtain Compound C-117 (3.7 g, yield: 38.5%).

| | MW | M.P. |
|---|---|---|
| C-117 | 516.56 | 286° C. |

### Example 3: Preparation of Compound C-118

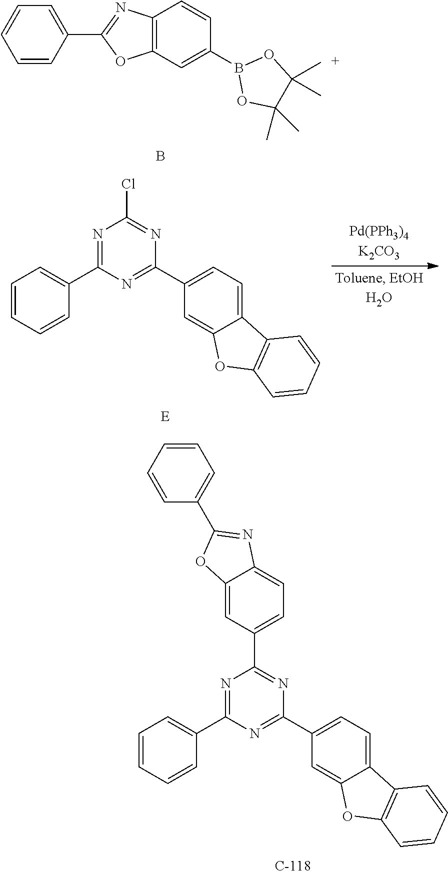

Compound B (6 g, 18.6 mmol), Compound E (6 g, 16.7 mmol), Pd(PPh₃)₄ (1.07 g, 0.92 mmol), and potassium carbonate (7.74 g, 56 mmol) were added to a reaction vessel, dissolved in 84 mL of toluene, 28 mL of ethanol, and 28 mL of water, and refluxed for 3 hours at 120° C. After the reaction was completed, the resulting solid was filtered, and washed with methanol. The filtrate was recrystallized by xylene and ethyl acetate to obtain Compound C-118 (5.2 g, yield: 54%).

| | MW | M.P. |
|---|---|---|
| C-118 | 516.56 | 305° C. |

### Example 4: Preparation of Compound C-180

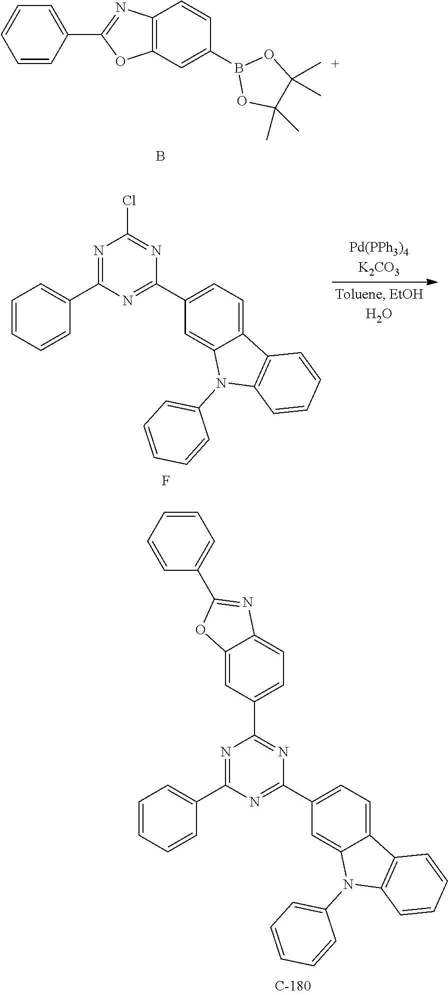

Compound B (3.7 g, 11.6 mmol), Compound E (5 g, 11.6 mmol), Pd(PPh₃)₄ (670 mg, 0.57 mmol), and potassium carbonate (4.8 g, 34.7 mmol) were added to a reaction vessel, dissolved in 60 mL of toluene, 30 mL of ethanol, and 30 mL of water, and refluxed for 3 hours at 120° C. After the reaction was completed, the resulting solid was filtered, and washed with methanol. The filtrate was recrystallized by xylene and ethyl acetate to obtain Compound C-180 (3.2 g, yield: 47%).

| | MW | M.P. |
|---|---|---|
| C-180 | 591.2 | 303° C. |

### Device Examples 1 to 10: Producing OLEDs Deposited with the Plurality of Host Materials According to the Present Disclosure

An OLED according to the present disclosure was produced. First, a transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and then was stored in isopropyl alcohol. The ITO substrate was mounted on a substrate holder of a vacuum vapor deposition apparatus. Compound HI-1 shown in Table 3 was introduced into a cell of the vacuum vapor deposition apparatus, and Compound HT-1 was introduced into another cell. The two materials were evaporated at different rates, and Compound HI-1 was deposited in a doping amount of 3 wt % based to the total amount of Compound HI-1 and Compound HT-1 to form a hole injection layer with a thickness of 10 nm. Subsequently, Compound HT-1 was deposited on the hole injection layer to form a first hole transport layer with a thickness of 80 nm. Next, Compound HT-2 was introduced into another cell of the vacuum vapor deposition apparatus and was evaporated by applying an electric current to the cell, thereby depositing a second hole transport layer with a thickness of 30 nm on the first hole transport layer. After forming the hole injection layer and the hole transport layers, a light-emitting layer was deposited thereon as follows: Each of the first host compound and the second host compound shown in Table 1 below were introduced into two cells of the vacuum vapor deposition apparatus as hosts, and Compound D-130 was introduced into another cell as a dopant. The two host materials were evaporated at a rate of 1:2 (first host:second host) and the dopant material was simultaneously evaporated at a different rate, and the dopant was deposited in a doping amount of 10 wt % based on the total amount of the hosts and dopant to form a light-emitting layer with a thickness of 40 nm on the second hole transport layer. Then, Compound ETL-1 and Compound EIL-1 were evaporated at a weight ratio of 40:60 as an electron transport material to form an electron transport layer having a thickness of 35 nm on the light-emitting layer. After depositing Compound EIL-1 as an electron injection layer with a thickness of 2 nm on the electron transport layer, an Al cathode was deposited with a thickness of 80 nm on the electron injection layer by using another vacuum vapor deposition apparatus, thereby producing an OLED. All the materials used for producing the OLED were purified by vacuum sublimation at 10⁻⁶ torr.

### Comparative Example 1: Producing an OLED Comprising a Comparative Compound as a Host

An OLED was produced in the same manner as in Device Examples 1 to 10, except that Compound C-1 was used alone as a host of a light-emitting layer.

The driving voltage, luminous efficiency, and light-emitting color at a luminance of 1,000 nit, and the time taken for luminance to reduce from 100% to 50% at a luminance of 60,000 nit (lifespan: T50) of the OLEDs produced in Device Examples 1 to 10 and Comparative Example 1 are shown in Table 1 below.

TABLE 1

| | | | Driving | Luminous | Light- | |
|---|---|---|---|---|---|---|
| | First | Second | Voltage | Efficiency | Emitting | Lifespan |
| | Host | Host | (V) | (cd/A) | Color | (T50, hr) |
| Device | C-117 | H2-146 | 2.7 | 100.6 | Green | 154 |
| Example 1 | | | | | | |
| Device | C-117 | H2-2-D20 | 2.7 | 100.3 | Green | 184 |
| Example 2 | | | | | | |
| Device | C-118 | H2-146 | 2.6 | 96.7 | Green | 148 |
| Example 3 | | | | | | |
| Device | C-118 | H2-2-D20 | 2.6 | 96.0 | Green | 185 |
| Example 4 | | | | | | |
| Device | C-116 | H2-146 | 2.7 | 93.6 | Green | 177 |
| Example 5 | | | | | | |
| Device | C-116 | H2-2-D20 | 2.6 | 93.0 | Green | 232 |
| Example 6 | | | | | | |
| Device | C-180 | H2-146 | 2.7 | 98.7 | Green | 142 |
| Example 7 | | | | | | |
| Device | C-180 | H2-2-D20 | 2.7 | 100.1 | Green | 182 |
| Example 8 | | | | | | |
| Device | C-48 | H2-146 | 2.9 | 101.6 | Green | 345 |
| Example 9 | | | | | | |
| Device | C-48 | H2-2-D20 | 2.8 | 102.6 | Green | 439 |
| Example 10 | | | | | | |
| Comparative | C-1 | | 4.4 | 44.4 | Green | — |
| Example 1 | | | | | | |

From Table 1 above, it can be confirmed that the OLEDs (Device Examples 1 to 10) comprising the plurality of host materials according to the present disclosure, exhibit lower driving voltage, higher luminous efficiency, and significantly improved lifespan characteristics compared to the OLED (Comparative Example 1) comprising a conventional host material. Since the OLED according to Comparative Example 1 could not achieve a luminance of 60,000 nit, it was impossible to measure the lifespan thereof.

### Device Examples 11 to 14: Producing OLEDs Deposited with the Plurality of Host Materials According to the Present Disclosure

An OLED according to the present disclosure was produced. First, a transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and then was stored in isopropyl alcohol. The ITO substrate was mounted on a substrate holder of a vacuum vapor deposition apparatus. Compound HI-1 shown in Table 3 was introduced into a cell of the vacuum vapor deposition apparatus, and Compound HT-1 was introduced into another cell. The two materials were evaporated at different rates, and Compound HI-1 was deposited in a doping amount of 3 wt % based to the total amount of Compound HI-1 and Compound HT-1 to form a hole injection layer with a thickness of 10 nm. Subsequently, Compound HT-1 was deposited on the hole injection layer to form a first hole transport layer with a thickness of 80 nm. Next, Compound HT-2 was introduced into another cell of the vacuum vapor deposition apparatus and was evaporated by applying an electric current to the cell, thereby depositing a second hole transport layer with a thickness of 30 nm on the first hole transport layer. After forming the hole injection layer and the hole transport layers, a light-emitting layer was deposited thereon as follows: the host compounds shown in Table 2 below were introduced into two cells of the vacuum vapor deposition apparatus as hosts, and Compound D-130 was introduced into another cell as a dopant. The dopant and host materials were simultaneously evaporated at different rates, and the dopant was deposited in a doping amount of 10 wt % based on the total amount of the hosts and dopant to form a light-emitting layer with a thickness of 40 nm on the second hole transport layer. Then, Compound ETL-1 and Compound EIL-1 were evaporated at a weight ratio of 40:60 as an electron transport material to form an electron transport layer having a thickness of 35 nm on the light-emitting layer. After depositing Compound EIL-1 as an electron injection layer with a thickness of 2 nm on the electron transport layer, an Al cathode was deposited with a thickness of 80 nm on the electron injection layer by using another vacuum vapor deposition apparatus, thereby producing an OLED. All the materials used for producing the OLED were purified by vacuum sublimation at 10⁻⁶ torr.

### Comparative Example 2: Producing an OLED Comprising a Comparative Compound as a Host

An OLED was produced in the same manner as in Device Examples 11 to 14, except that Compound A was used alone as a host of a light-emitting layer.

The driving voltage, luminous efficiency, and light-emitting color at a luminance of 1,000 nit, and the time taken for luminance to reduce from 100% to 50% at a luminance of 20,000 nit (lifespan: T50) of the OLEDs produced in Device Examples 11 to 14 and Comparative Example 2 are shown in the following Table 2.

TABLE 2

| | | Driving | Luminous | Light- | |
|---|---|---|---|---|---|
| | | Voltage | Efficiency | Emitting | Lifespan |
| | Host | (V) | (cd/A) | Color | (T50, hr) |
| Device Example | C-117 | 3.2 | 61.5 | Green | 102 |
| 11 | | | | | |
| Device Example | C-118 | 2.8 | 45.0 | Green | 88.3 |
| 12 | | | | | |
| Device Example | C-116 | 2.6 | 64.9 | Green | 224 |
| 13 | | | | | |
| Device Example | C-180 | 2.5 | 59.2 | Green | 120 |
| 14 | | | | | |
| Comparative | A | 3.4 | 44.2 | Green | 33.7 |
| Example 2 | | | | | |

From Table 2 above, it can be confirmed that the OLEDs (Device Examples 11 to 14) comprising host compounds according to the present disclosure, exhibit lower driving voltage, higher luminous efficiency, and significantly improved lifespan characteristics compared to the OLED (Comparative Example 2) comprising a conventional host compound.

The compounds used in the Device Examples and the Comparative Examples are shown in Table 3 below.

TABLE 3

| | |
|---|---|
| Hole Injection Layer/ Hole Transport Layer | 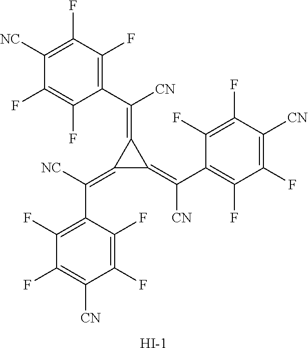 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; [I-]] |
| | 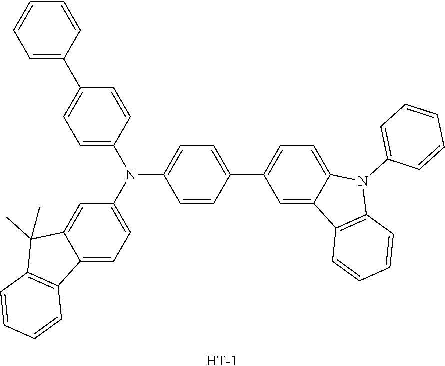 |
| | 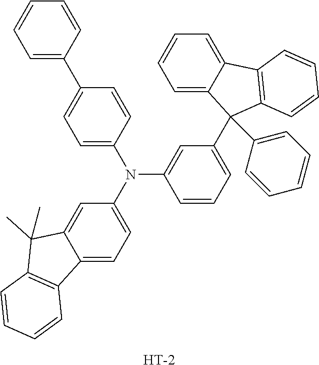 |
| Light- Emitting Layer | 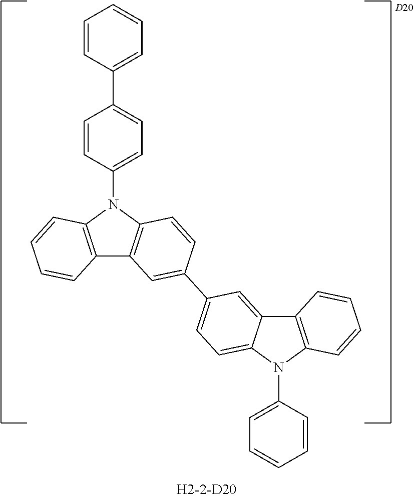 |
| | 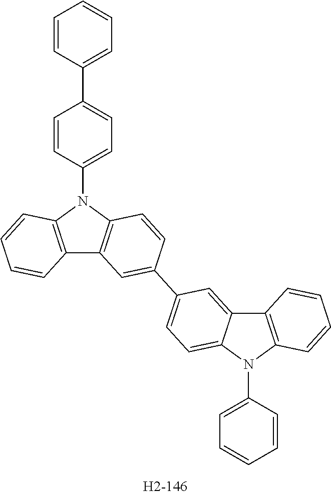 [SMILES: c1ccc(-c2ccc(-n3c4ccccc4c4cc(-c5ccc6c(c5)c5ccccc5n6-c5ccccc5)ccc43)cc2)cc1] |
| | 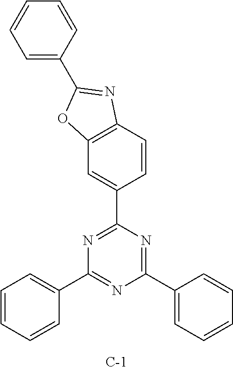 |
| | 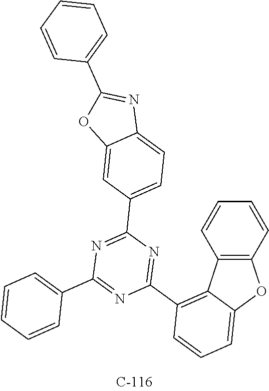 [SMILES: c1ccc(-c2nc(-c3ccc4nc(-c5ccccc5)oc4c3)nc(-c3cccc4oc5ccccc5c34)n2)cc1] |
| | 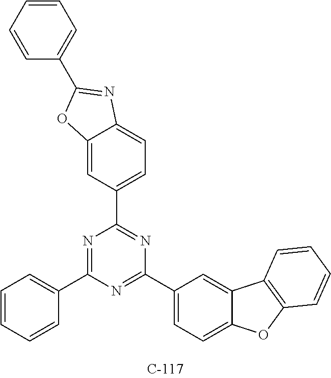 [SMILES: c1ccc(-c2nc(-c3ccc4nc(-c5ccccc5)oc4c3)nc(-c3ccc4oc5ccccc5c4c3)n2)cc1] |
| | 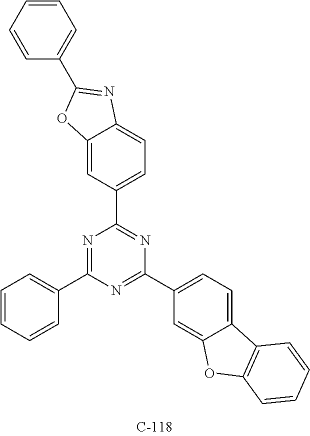 [SMILES: c1ccc(-c2nc(-c3ccc4nc(-c5ccccc5)oc4c3)nc(-c3ccc4c(c3)oc3ccccc34)n2)cc1] |
| | 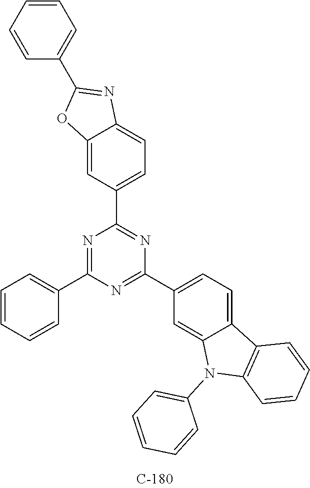 [SMILES: c1ccc(-c2nc(-c3ccc4nc(-c5ccccc5)oc4c3)nc(-c3ccc4c5ccccc5n(-c5ccccc5)c4c3)n2)cc1] |
| | 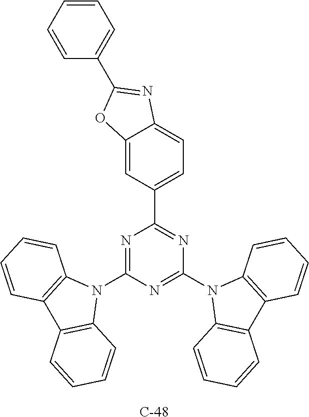 |
| | 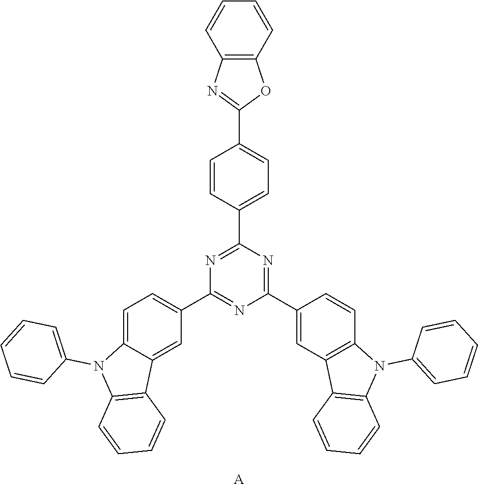 |
| | 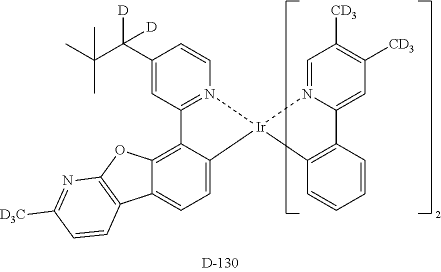 |
| Electron Transport Layer/ Electron Injection Layer | 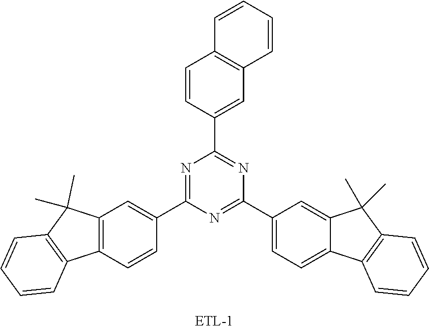 [SMILES: CC1(C)c2ccccc2-c2ccc(-c3nc(-c4ccc5c(c4)C(C)(C)c4ccccc4-5)nc(-c4ccc5ccccc5c4)n3)cc21] |
| | 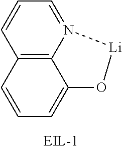 |

1. A plurality of host materials comprising at least one first host compound and at least one second host compound, wherein the first host compound is represented by the following formula 1, and the second host compound is represented by the following formula 2: 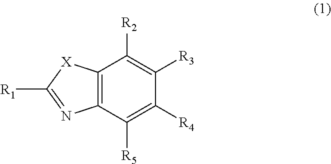
in formula 1,
X represents O or S;
R₁ to R₅ each independently represent hydrogen, deuterium, a substituted or unsubstituted (C6-C30)aryl, or the following formula a; with a proviso that at least one of R₁ to R₅ represents formula a; 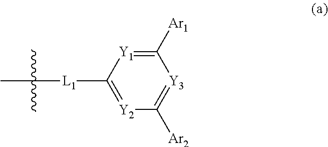
in formula a,
L₁ represents a single bond, a substituted or unsubstituted (C6-C30)arylene, or a substituted or unsubstituted (3- to 30-membered)heteroarylene;
Y₁ to Y₃ each independently represent N or CH; with a proviso that at least two of Y₁ to Y₃ represent N; and
Ar₁ and Ar₂ each independently represent a substituted or unsubstituted (C1-C30)alkyl, a substituted or unsubstituted (C6-C30)aryl, or a substituted or unsubstituted (3- to 30-membered)heteroaryl; 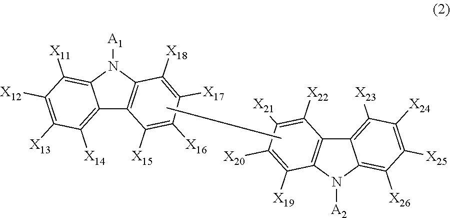
in formula 2,
A₁ and A₂ each independently represent a substituted or unsubstituted (C6-C30)aryl, a substituted or unsubstituted dibenzofuranyl, a substituted or unsubstituted dibenzothiophenyl, or a substituted or unsubstituted carbazolyl;
any one of X₁₅ to X₁₈ and any one of X₁₉ to X₂₂ are linked to each other to form a single bond; and
X₁₁ to X₁₄, X₂₃ to X₂₆, and X₁₅ to X₂₂ not forming the single bond each independently represent hydrogen, deuterium, a substituted or unsubstituted (C6-C30)aryl, or a substituted or unsubstituted (3- to 30-membered)heteroaryl; or may be linked to an adjacent substituent(s) to form a ring(s).

2. The plurality of host materials according to claim 1, wherein Ar₁ and Ar₂ of formula a each independently represent a phenyl unsubstituted or substituted with a dibenzofuranyl(s), a dibenzothiophenyl(s), a carbazolyl(s), or a 9-phenylcarbazolyl(s); a biphenyl; a terphenyl; a quaterphenyl; a naphthyl; a phenylnaphthyl; a naphthylphenyl; a pyridyl; a pyrimidinyl; a triazinyl; a dibenzofuranyl unsubstituted or substituted with a phenyl(s); a dibenzothiophenyl unsubstituted or substituted with a phenyl(s); or a carbazolyl unsubstituted or substituted with at least one of a phenyl(s) and a biphenyl(s), which may be further substituted with at least one deuterium.

3. The plurality of host materials according to claim 1, wherein in formula 2, at least one of X₁₁, X₁₈, X₁₉ and X₂₆ represents deuterium.

4. The plurality of host materials according to claim 1, wherein the compound represented by formula 2 has a deuterium substitution rate of 40% to 100%.

5. The plurality of host materials according to claim 1, wherein in formula 2, a deuterium substitution rate of X₁ to X₂₆ is 25% to 100%.

6. The plurality of host materials according to claim 1, wherein formula 2 is represented by at least one of the following formulas 2-1 to 2-8: 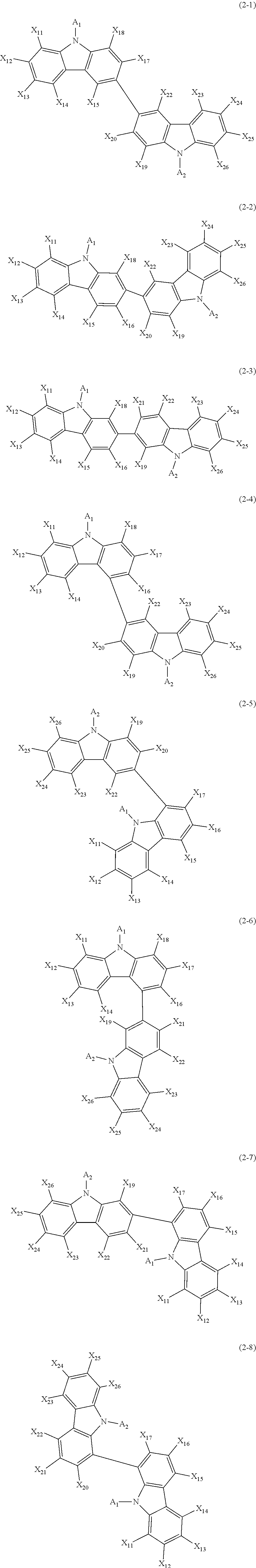
in formulas 2-1 to 2-8,
A₁, A₂, and X₁₁ to X₂₆ are as defined in claim 1.

7. The plurality of host materials according to claim 1, wherein A₁ and A₂ of formula 2 each independently represent a substituted or unsubstituted phenyl, a substituted or unsubstituted biphenyl, a substituted or unsubstituted terphenyl, a substituted or unsubstituted naphthyl, a substituted or unsubstituted fluorenyl, a substituted or unsubstituted benzofluorenyl, a substituted or unsubstituted triphenylenyl, a substituted or unsubstituted fluoranthenyl, a substituted or unsubstituted phenanthrenyl, a substituted or unsubstituted dibenzofuranyl, a substituted or unsubstituted carbazolyl, or a substituted or unsubstituted dibenzothiophenyl.

8. The plurality of host materials according to claim 1, wherein the substituent(s) of the substituted alkyl, the substituted aryl(ene), the substituted heteroaryl(ene), the substituted dibenzofuranyl, the substituted dibenzothiophenyl, and the substituted carbazolyl, each independently, are at least one selected from the group consisting of deuterium; a halogen; a cyano; a carboxyl; a nitro; a hydroxyl; a phosphine oxide; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (3- to 30-membered)heteroaryl unsubstituted or substituted with at least one of deuterium and a (C6-C30)aryl(s); a (C6-C30)aryl unsubstituted or substituted with deuterium; a tri(C1-C30)alkylsilyl; a tri(C6-C30)arylsilyl; a di(C1-C30)alkyl(C6-C30)arylsilyl; a (C1-C30)alkyldi(C6-C30)arylsilyl; an amino; a mono- or di-(C1-C30)alkylamino; a mono- or di-(C2-C30)alkenylamino; a mono- or di-(C6-C30)arylamino; a mono- or di-(3- to 30-membered)heteroarylamino; a (C1-C30)alkyl(C2-C30)alkenylamino; a (C1-C30)alkyl(C6-C30)arylamino; a (C1-C30)alkyl(3- to 30-membered)heteroarylamino; a (C2-C30)alkenyl(C6-C30)arylamino; a (C2-C30)alkenyl(3- to 30-membered)heteroarylamino; a (C6-C30)aryl(3- to 30-membered)heteroarylamino; a (C1-C30)alkylcarbonyl; a (C1-C30)alkoxycarbonyl; a (C6-C30)arylcarbonyl; a (C6-C30)arylphosphine; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl.

9. The plurality of host materials according to claim 1, wherein the compound represented by formula 1 is at least one selected from the following compounds:

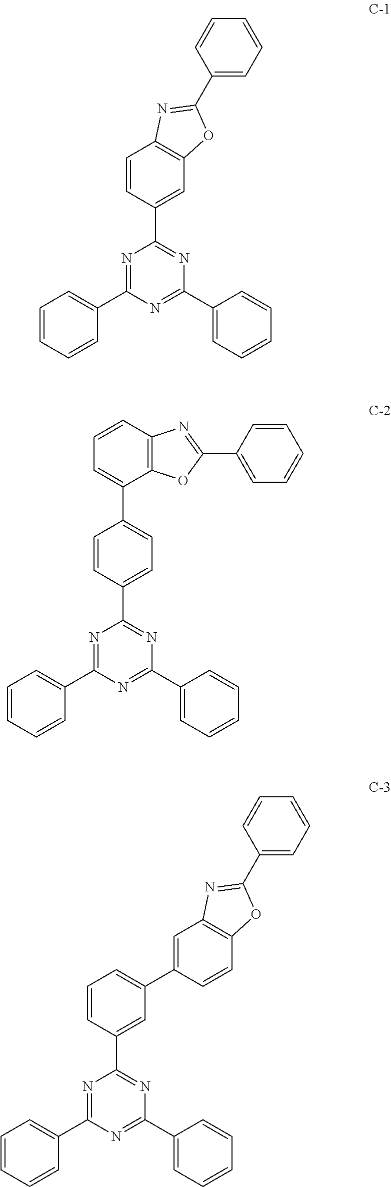 [SMILES: c1ccc(-c2nc(-c3ccccc3)nc(-c3ccc4nc(-c5ccccc5)oc4c3)n2)cc1 ; c1ccc(-c2nc(-c3ccccc3)nc(-c3ccc(-c4cccc5nc(-c6ccccc6)oc45)cc3)n2)cc1 ; c1ccc(-c2nc(-c3ccccc3)nc(-c3cccc(-c4ccc5oc(-c6ccccc6)nc5c4)c3)n2)cc1]

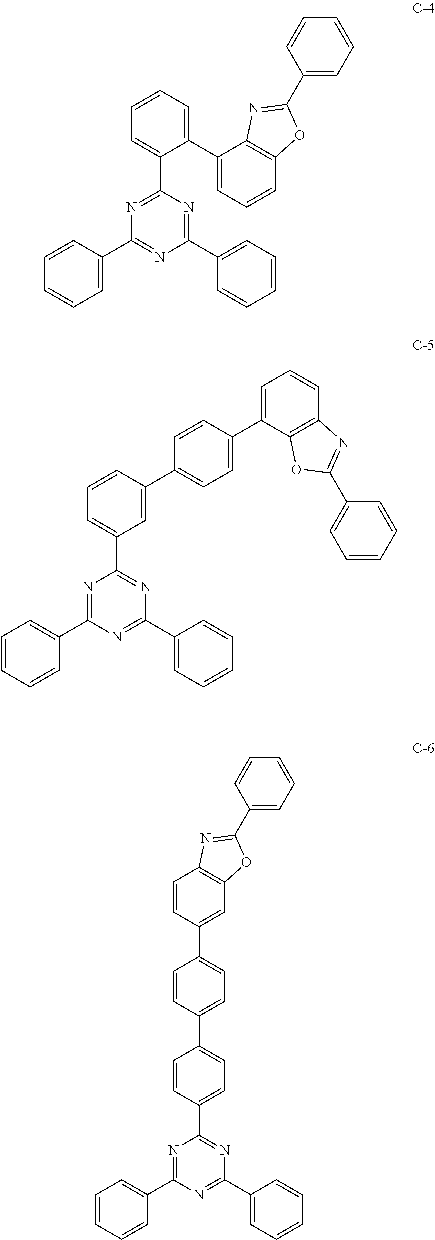 [SMILES: c1ccc(-c2nc(-c3ccccc3)nc(-c3ccccc3-c3cccc4oc(-c5ccccc5)nc34)n2)cc1 ; c1ccc(-c2nc(-c3ccccc3)nc(-c3cccc(-c4ccc(-c5cccc6nc(-c7ccccc7)oc56)cc4)c3)n2)cc1 ; c1ccc(-c2nc(-c3ccccc3)nc(-c3ccc(-c4ccc(-c5ccc6nc(-c7ccccc7)oc6c5)cc4)cc3)n2)cc1]

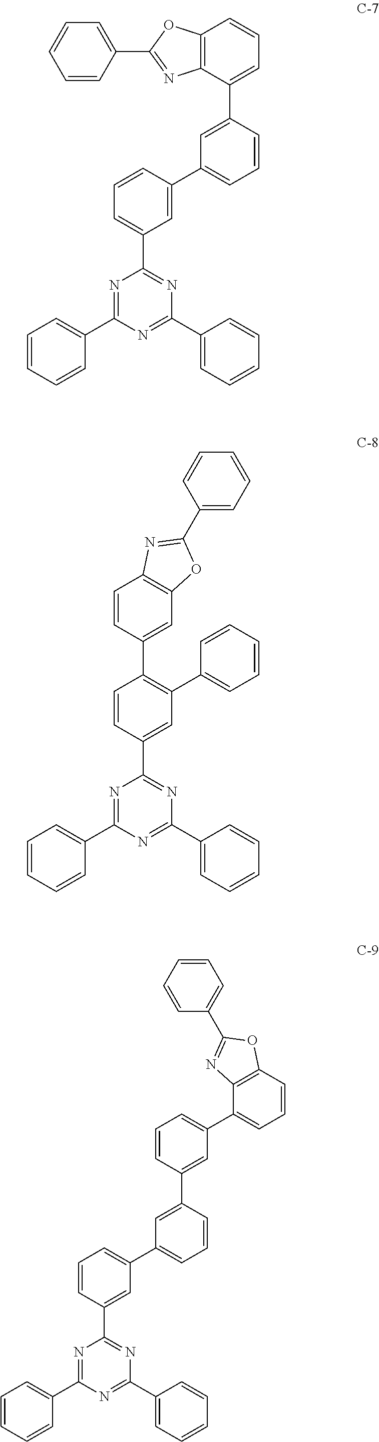 [SMILES: c1ccc(-c2nc(-c3ccccc3)nc(-c3cccc(-c4cccc(-c5cccc6oc(-c7ccccc7)nc56)c4)c3)n2)cc1 ; c1ccc(-c2nc(-c3ccccc3)nc(-c3ccc(-c4ccc5nc(-c6ccccc6)oc5c4)c(-c4ccccc4)c3)n2)cc1 ; c1ccc(-c2nc(-c3ccccc3)nc(-c3cccc(-c4cccc(-c5cccc(-c6cccc7oc(-c8ccccc8)nc67)c5)c4)c3)n2)cc1]

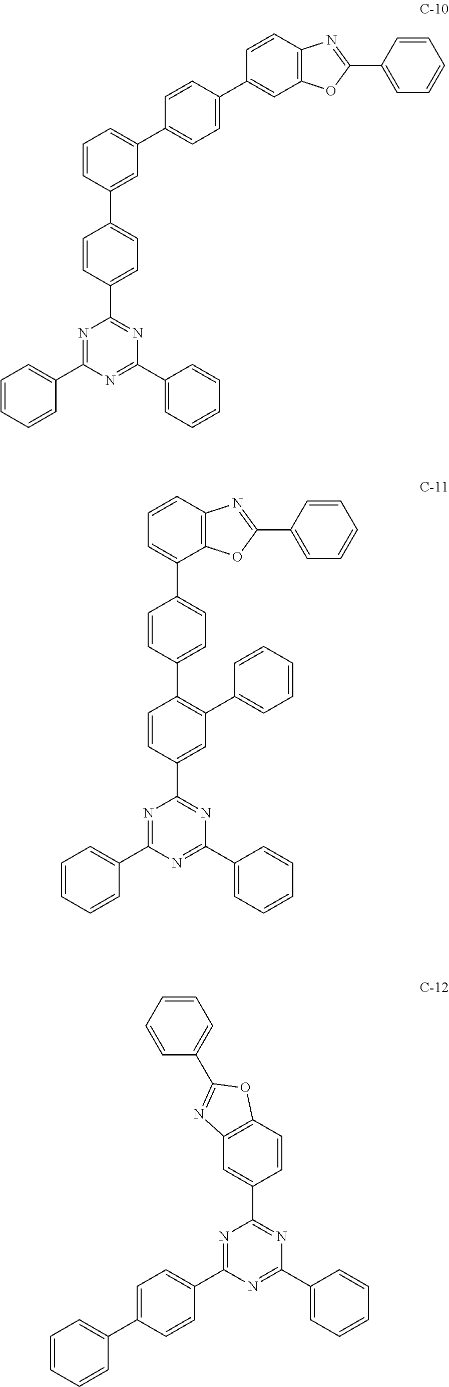 [SMILES: c1ccc(-c2nc(-c3ccccc3)nc(-c3ccc(-c4cccc(-c5ccc(-c6ccc7nc(-c8ccccc8)oc7c6)cc5)c4)cc3)n2)cc1 ; c1ccc(-c2nc(-c3ccccc3)nc(-c3ccc(-c4ccc(-c5cccc6nc(-c7ccccc7)oc56)cc4)c(-c4ccccc4)c3)n2)cc1 ; c1ccc(-c2ccc(-c3nc(-c4ccccc4)nc(-c4ccc5oc(-c6ccccc6)nc5c4)n3)cc2)cc1]

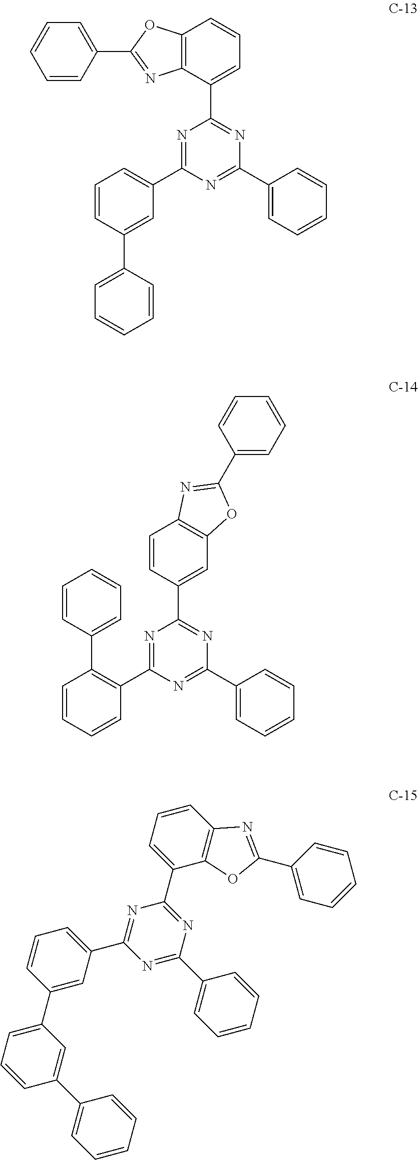 [SMILES: c1ccc(-c2cccc(-c3nc(-c4ccccc4)nc(-c4cccc5oc(-c6ccccc6)nc45)n3)c2)cc1 ; c1ccc(-c2nc(-c3ccc4nc(-c5ccccc5)oc4c3)nc(-c3ccccc3-c3ccccc3)n2)cc1 ; c1ccc(-c2cccc(-c3cccc(-c4nc(-c5ccccc5)nc(-c5cccc6nc(-c7ccccc7)oc56)n4)c3)c2)cc1]

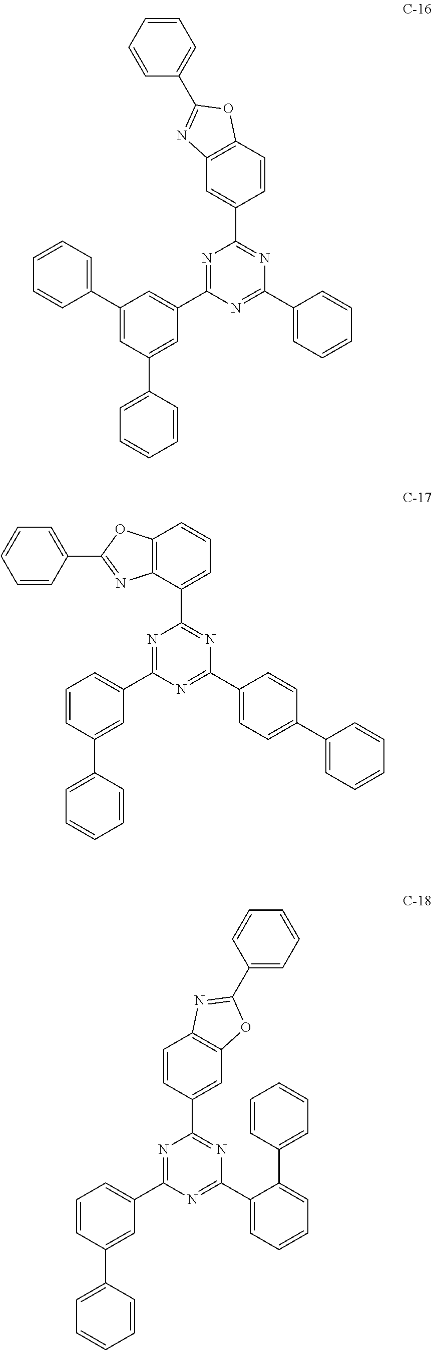 [SMILES: c1ccc(-c2cc(-c3ccccc3)cc(-c3nc(-c4ccccc4)nc(-c4ccc5oc(-c6ccccc6)nc5c4)n3)c2)cc1 ; c1ccc(-c2ccc(-c3nc(-c4cccc(-c5ccccc5)c4)nc(-c4cccc5oc(-c6ccccc6)nc45)n3)cc2)cc1 ; c1ccc(-c2cccc(-c3nc(-c4ccc5nc(-c6ccccc6)oc5c4)nc(-c4ccccc4-c4ccccc4)n3)c2)cc1]

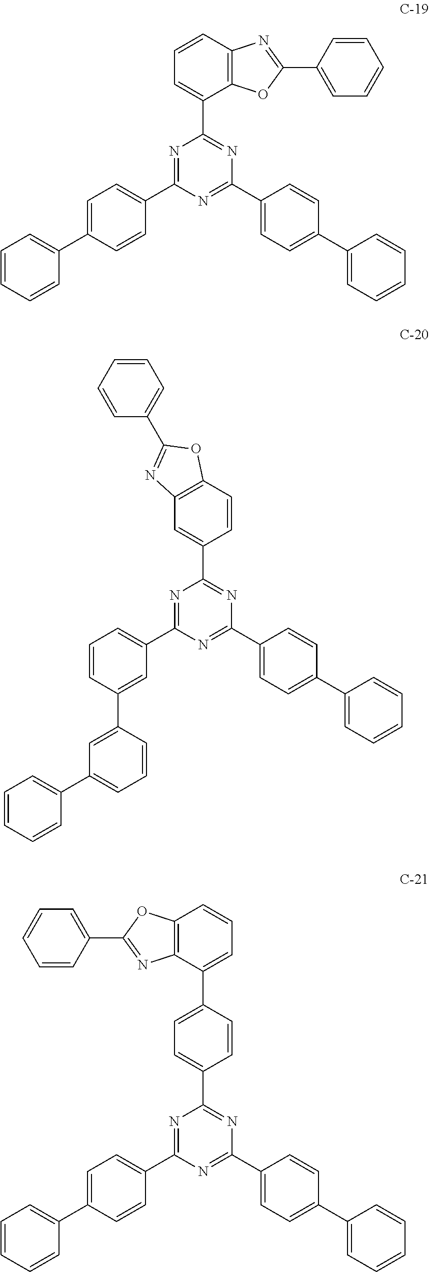 [SMILES: c1ccc(-c2ccc(-c3nc(-c4ccc(-c5ccccc5)cc4)nc(-c4cccc5nc(-c6ccccc6)oc45)n3)cc2)cc1 ; c1ccc(-c2ccc(-c3nc(-c4cccc(-c5cccc(-c6ccccc6)c5)c4)nc(-c4ccc5oc(-c6ccccc6)nc5c4)n3)cc2)cc1 ; c1ccc(-c2ccc(-c3nc(-c4ccc(-c5ccccc5)cc4)nc(-c4ccc(-c5cccc6oc(-c7ccccc7)nc56)cc4)n3)cc2)cc1]

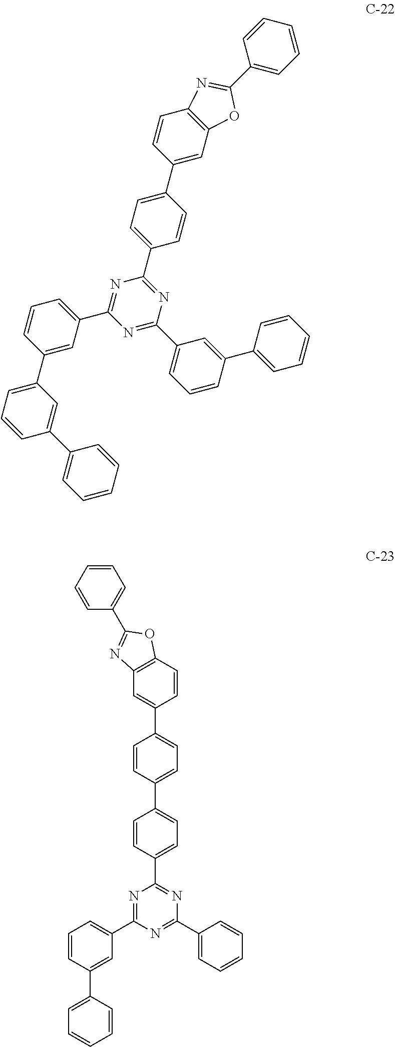 [SMILES: c1ccc(-c2cccc(-c3cccc(-c4nc(-c5ccc(-c6ccc7nc(-c8ccccc8)oc7c6)cc5)nc(-c5cccc(-c6ccccc6)c5)n4)c3)c2)cc1 ; c1ccc(-c2cccc(-c3nc(-c4ccccc4)nc(-c4ccc(-c5ccc(-c6ccc7oc(-c8ccccc8)nc7c6)cc5)cc4)n3)c2)cc1]

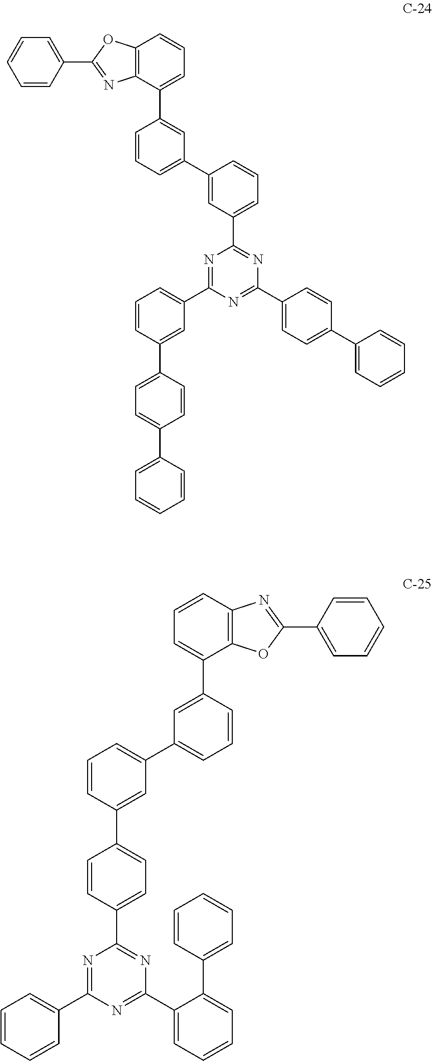 [SMILES: c1ccc(-c2ccc(-c3cccc(-c4nc(-c5ccc(-c6ccccc6)cc5)nc(-c5cccc(-c6cccc(-c7cccc8oc(-c9ccccc9)nc78)c6)c5)n4)c3)cc2)cc1 ; c1ccc(-c2nc(-c3ccc(-c4cccc(-c5cccc(-c6cccc7nc(-c8ccccc8)oc67)c5)c4)cc3)nc(-c3ccccc3-c3ccccc3)n2)cc1]

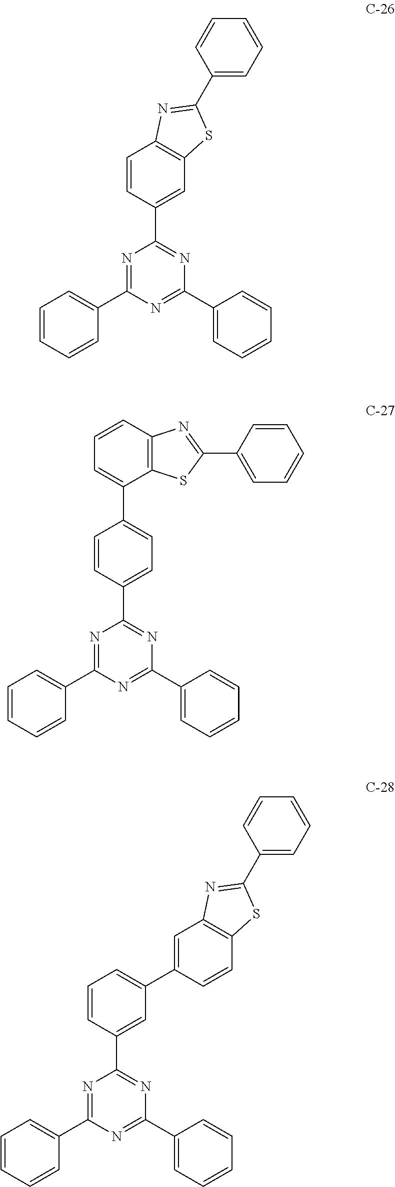 [SMILES: c1ccc(-c2nc(-c3ccccc3)nc(-c3ccc4nc(-c5ccccc5)sc4c3)n2)cc1 ; c1ccc(-c2nc(-c3ccccc3)nc(-c3ccc(-c4cccc5nc(-c6ccccc6)sc45)cc3)n2)cc1 ; c1ccc(-c2nc(-c3ccccc3)nc(-c3cccc(-c4ccc5sc(-c6ccccc6)nc5c4)c3)n2)cc1]

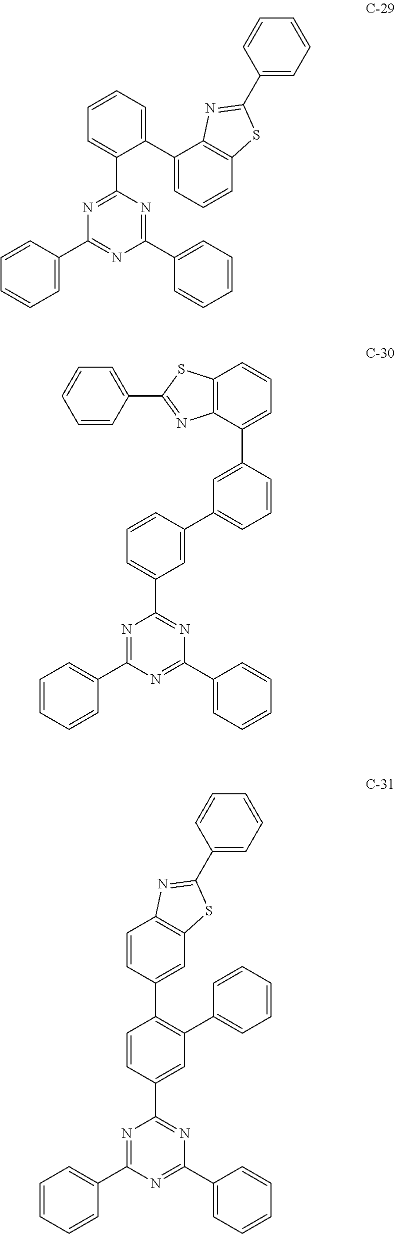 [SMILES: c1ccc(-c2nc(-c3ccccc3)nc(-c3ccccc3-c3cccc4sc(-c5ccccc5)nc34)n2)cc1 ; c1ccc(-c2nc(-c3ccccc3)nc(-c3cccc(-c4cccc(-c5cccc6sc(-c7ccccc7)nc56)c4)c3)n2)cc1 ; c1ccc(-c2nc(-c3ccccc3)nc(-c3ccc(-c4ccc5nc(-c6ccccc6)sc5c4)c(-c4ccccc4)c3)n2)cc1]

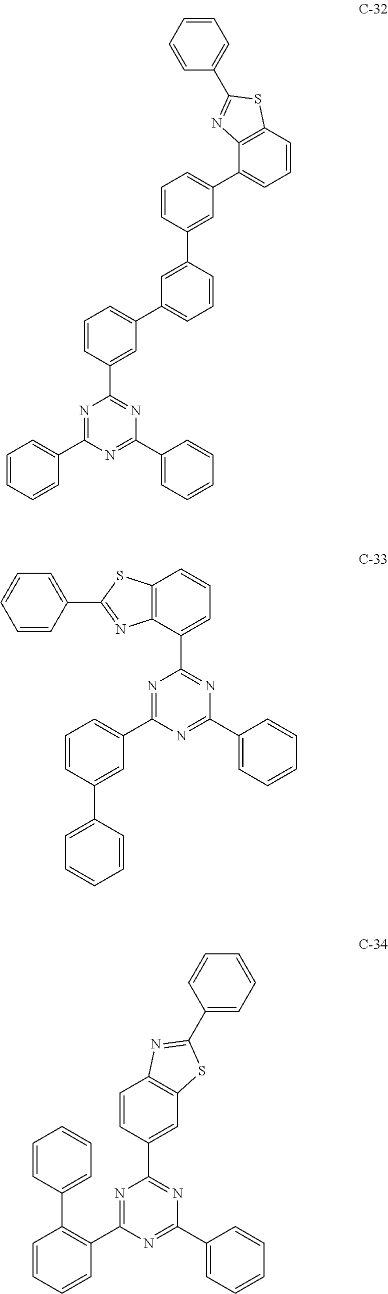 [SMILES: c1ccc(-c2nc(-c3ccccc3)nc(-c3cccc(-c4cccc(-c5cccc(-c6cccc7sc(-c8ccccc8)nc67)c5)c4)c3)n2)cc1 ; c1ccc(-c2cccc(-c3nc(-c4ccccc4)nc(-c4cccc5sc(-c6ccccc6)nc45)n3)c2)cc1 ; c1ccc(-c2nc(-c3ccc4nc(-c5ccccc5)sc4c3)nc(-c3ccccc3-c3ccccc3)n2)cc1]

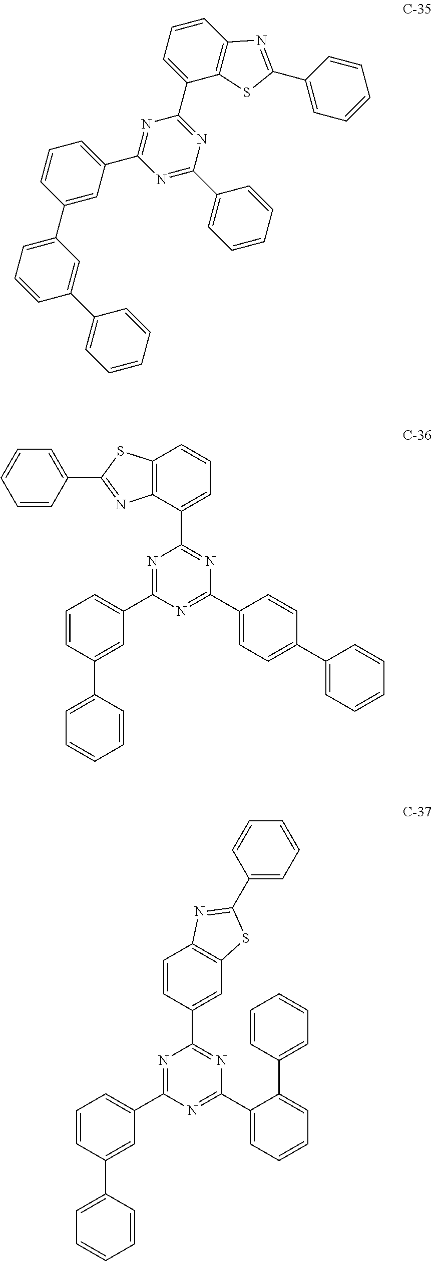 [SMILES: c1ccc(-c2cccc(-c3cccc(-c4nc(-c5ccccc5)nc(-c5cccc6nc(-c7ccccc7)sc56)n4)c3)c2)cc1 ; c1ccc(-c2ccc(-c3nc(-c4cccc(-c5ccccc5)c4)nc(-c4cccc5sc(-c6ccccc6)nc45)n3)cc2)cc1 ; c1ccc(-c2cccc(-c3nc(-c4ccc5nc(-c6ccccc6)sc5c4)nc(-c4ccccc4-c4ccccc4)n3)c2)cc1]

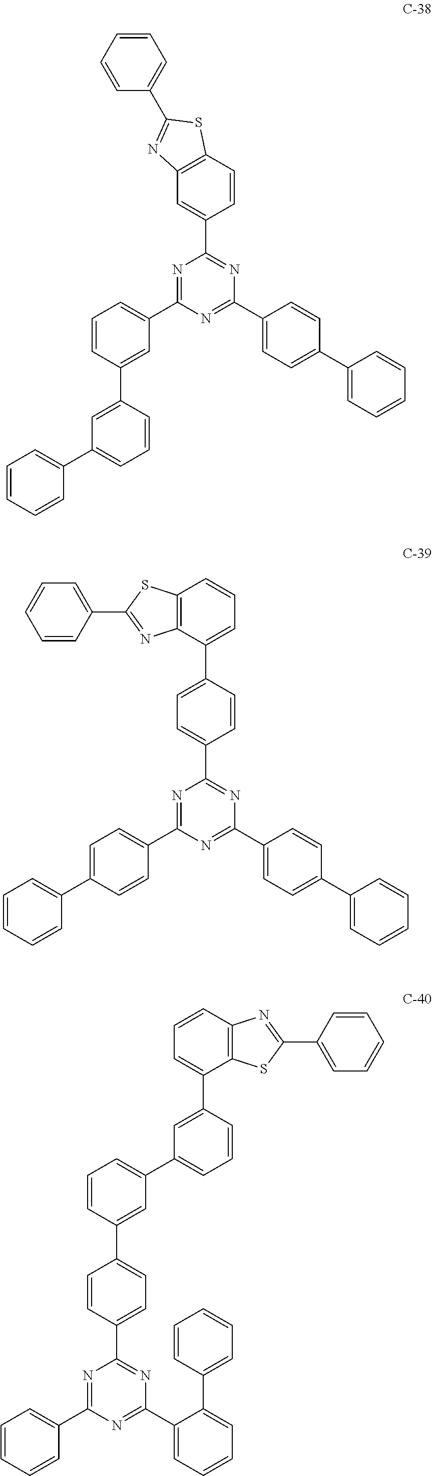 [SMILES: c1ccc(-c2ccc(-c3nc(-c4cccc(-c5cccc(-c6ccccc6)c5)c4)nc(-c4ccc5sc(-c6ccccc6)nc5c4)n3)cc2)cc1 ; c1ccc(-c2ccc(-c3nc(-c4ccc(-c5ccccc5)cc4)nc(-c4ccc(-c5cccc6sc(-c7ccccc7)nc56)cc4)n3)cc2)cc1 ; c1ccc(-c2nc(-c3ccc(-c4cccc(-c5cccc(-c6cccc7nc(-c8ccccc8)sc67)c5)c4)cc3)nc(-c3ccccc3-c3ccccc3)n2)cc1]

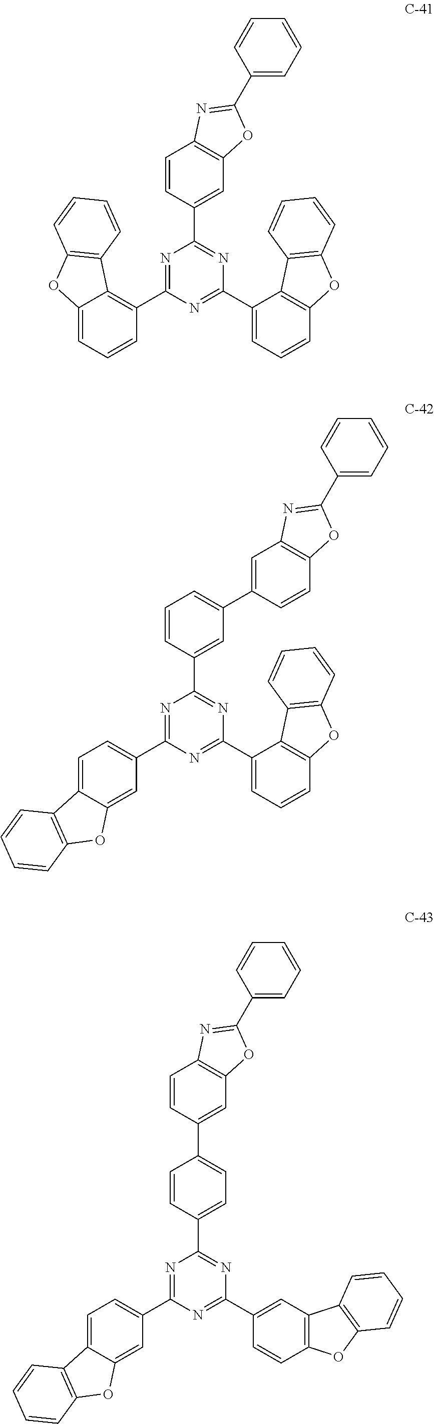 [SMILES: c1ccc(-c2nc3ccc(-c4nc(-c5cccc6oc7ccccc7c56)nc(-c5cccc6oc7ccccc7c56)n4)cc3o2)cc1 ; c1ccc(-c2nc3cc(-c4cccc(-c5nc(-c6ccc7c(c6)oc6ccccc67)nc(-c6cccc7oc8ccccc8c67)n5)c4)ccc3o2)cc1 ; c1ccc(-c2nc3ccc(-c4ccc(-c5nc(-c6ccc7c(c6)oc6ccccc67)nc(-c6ccc7oc8ccccc8c7c6)n5)cc4)cc3o2)cc1]

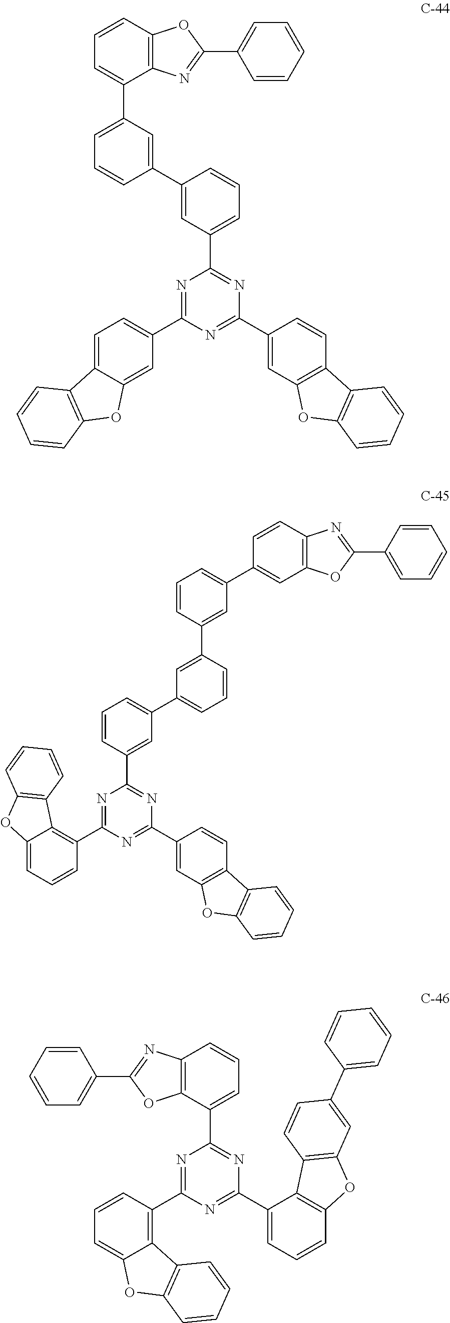 [SMILES: c1ccc(-c2nc3c(-c4cccc(-c5cccc(-c6nc(-c7ccc8c(c7)oc7ccccc78)nc(-c7ccc8c(c7)oc7ccccc78)n6)c5)c4)cccc3o2)cc1 ; c1ccc(-c2nc3ccc(-c4cccc(-c5cccc(-c6cccc(-c7nc(-c8ccc9c(c8)oc8ccccc89)nc(-c8cccc9oc%10ccccc%10c89)n7)c6)c5)c4)cc3o2)cc1 ; c1ccc(-c2ccc3c(c2)oc2cccc(-c4nc(-c5cccc6nc(-c7ccccc7)oc56)nc(-c5cccc6oc7ccccc7c56)n4)c23)cc1]

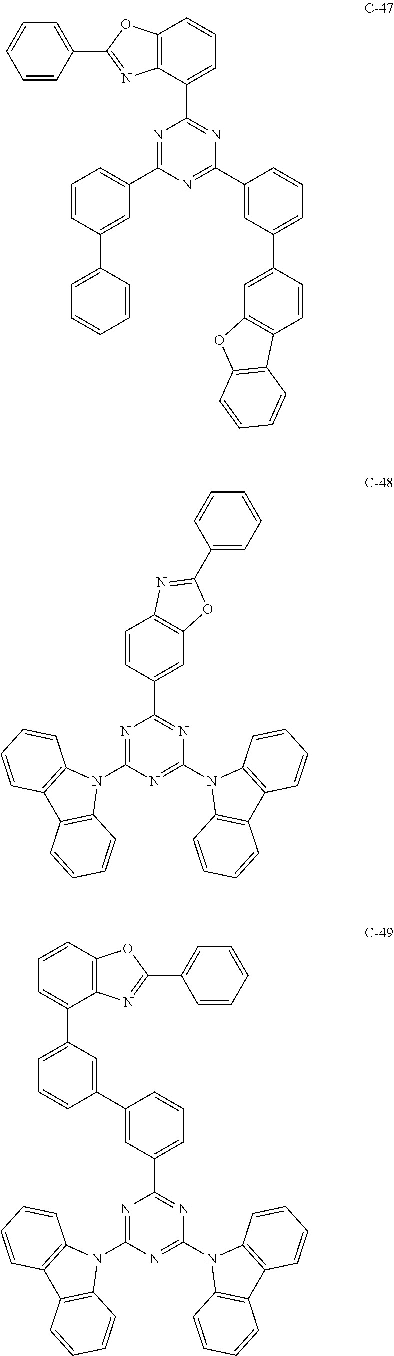 [SMILES: c1ccc(-c2cccc(-c3nc(-c4cccc(-c5ccc6c(c5)oc5ccccc56)c4)nc(-c4cccc5oc(-c6ccccc6)nc45)n3)c2)cc1 ; c1ccc(-c2nc3ccc(-c4nc(-n5c6ccccc6c6ccccc65)nc(-n5c6ccccc6c6ccccc65)n4)cc3o2)cc1 ; c1ccc(-c2nc3c(-c4cccc(-c5cccc(-c6nc(-n7c8ccccc8c8ccccc87)nc(-n7c8ccccc8c8ccccc87)n6)c5)c4)cccc3o2)cc1]

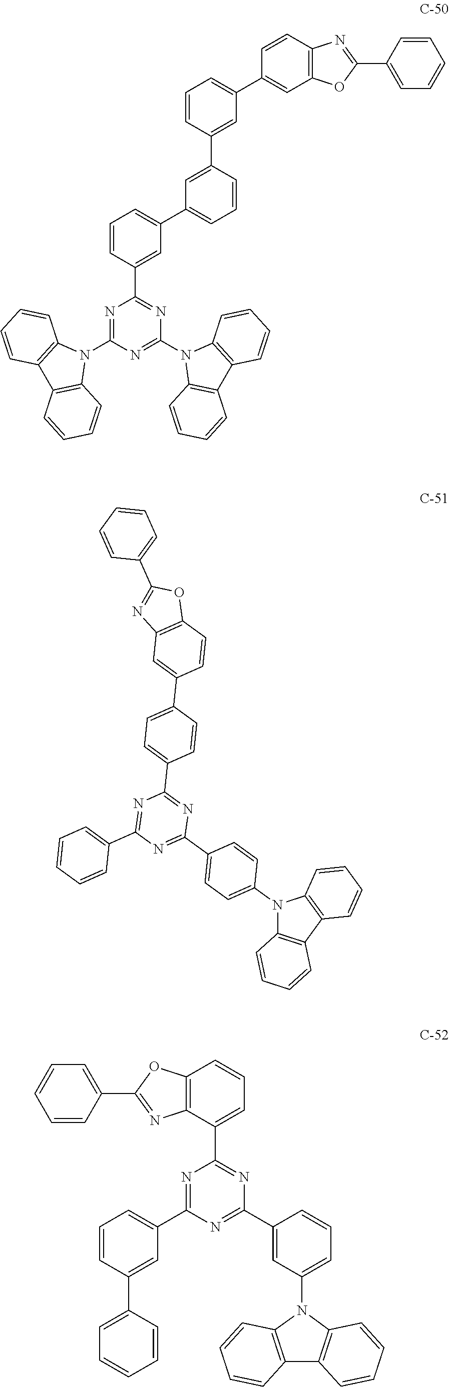 [SMILES: c1ccc(-c2nc3ccc(-c4cccc(-c5cccc(-c6cccc(-c7nc(-n8c9ccccc9c9ccccc98)nc(-n8c9ccccc9c9ccccc98)n7)c6)c5)c4)cc3o2)cc1 ; c1ccc(-c2nc(-c3ccc(-c4ccc5oc(-c6ccccc6)nc5c4)cc3)nc(-c3ccc(-n4c5ccccc5c5ccccc54)cc3)n2)cc1 ; c1ccc(-c2cccc(-c3nc(-c4cccc(-n5c6ccccc6c6ccccc65)c4)nc(-c4cccc5oc(-c6ccccc6)nc45)n3)c2)cc1]

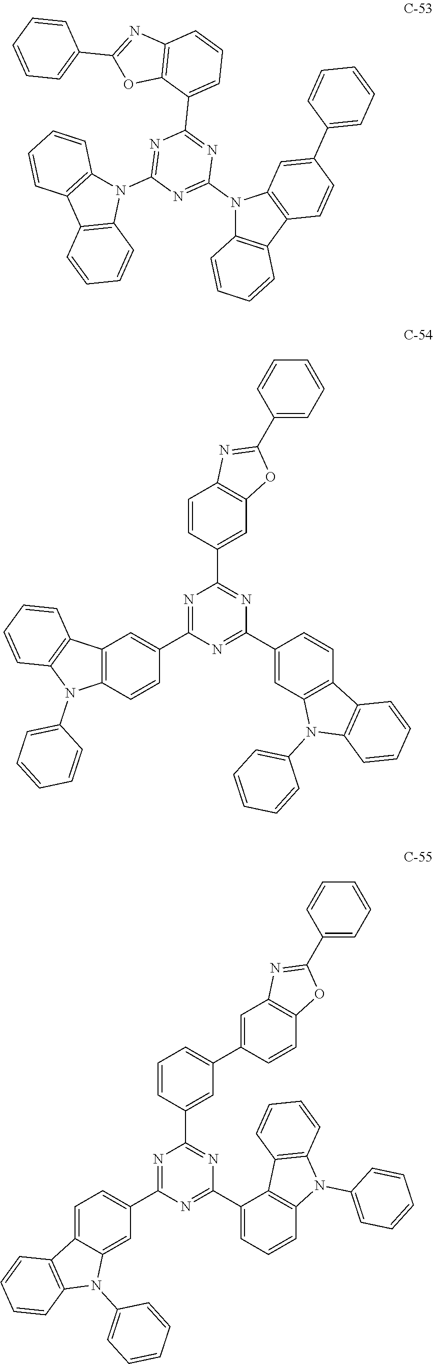 [SMILES: c1ccc(-c2ccc3c4ccccc4n(-c4nc(-c5cccc6nc(-c7ccccc7)oc56)nc(-n5c6ccccc6c6ccccc65)n4)c3c2)cc1 ; c1ccc(-c2nc3ccc(-c4nc(-c5ccc6c(c5)c5ccccc5n6-c5ccccc5)nc(-c5ccc6c7ccccc7n(-c7ccccc7)c6c5)n4)cc3o2)cc1 ; c1ccc(-c2nc3cc(-c4cccc(-c5nc(-c6ccc7c8ccccc8n(-c8ccccc8)c7c6)nc(-c6cccc7c6c6ccccc6n7-c6ccccc6)n5)c4)ccc3o2)cc1]

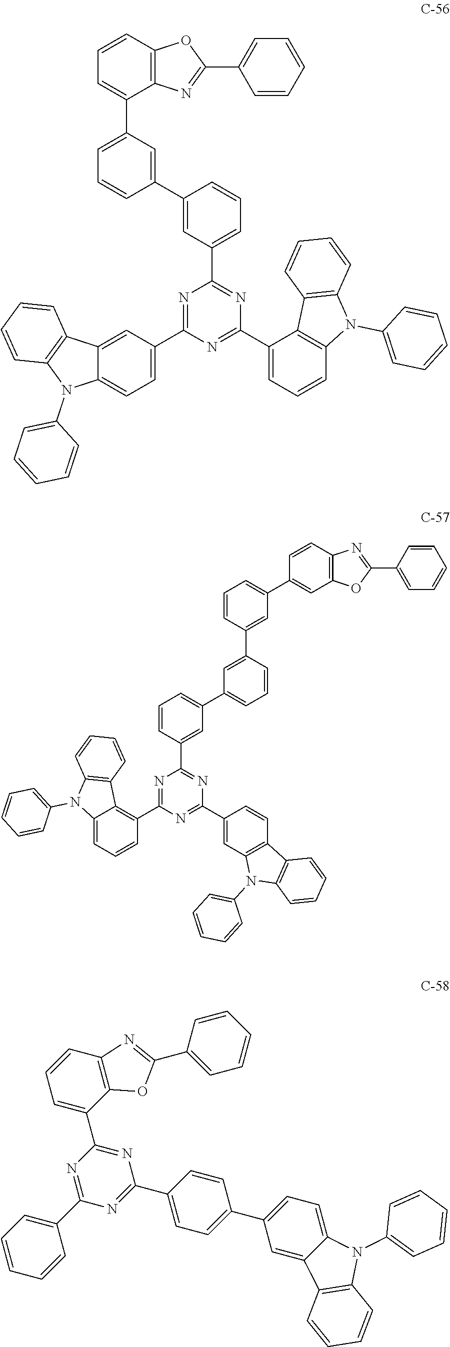 [SMILES: c1ccc(-c2nc3c(-c4cccc(-c5cccc(-c6nc(-c7ccc8c(c7)c7ccccc7n8-c7ccccc7)nc(-c7cccc8c7c7ccccc7n8-c7ccccc7)n6)c5)c4)cccc3o2)cc1 ; c1ccc(-c2nc3ccc(-c4cccc(-c5cccc(-c6cccc(-c7nc(-c8ccc9c%10ccccc%10n(-c%10ccccc%10)c9c8)nc(-c8cccc9c8c8ccccc8n9-c8ccccc8)n7)c6)c5)c4)cc3o2)cc1 ; c1ccc(-c2nc(-c3ccc(-c4ccc5c(c4)c4ccccc4n5-c4ccccc4)cc3)nc(-c3cccc4nc(-c5ccccc5)oc34)n2)cc1]

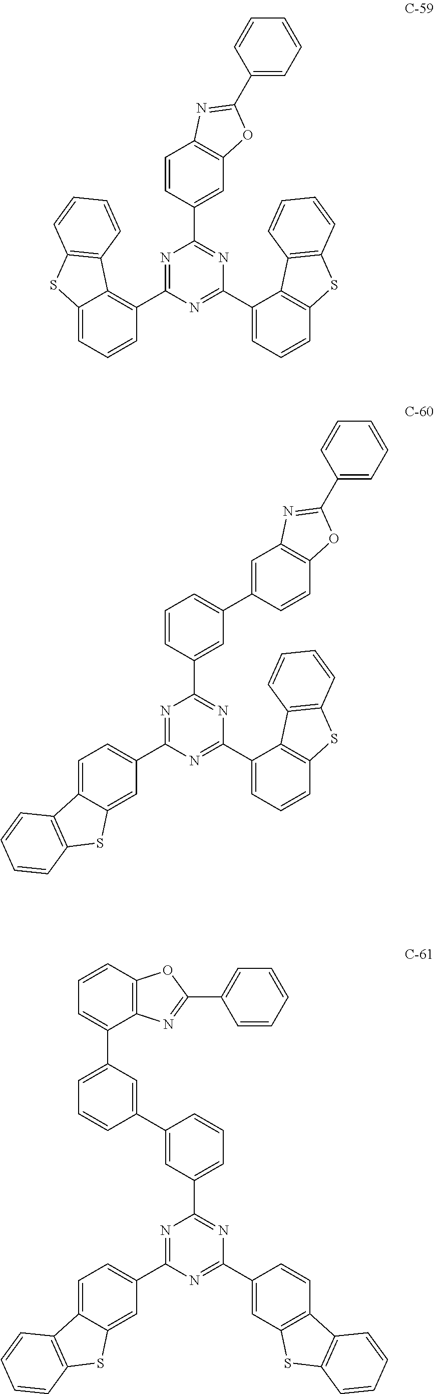 [SMILES: c1ccc(-c2nc3ccc(-c4nc(-c5cccc6sc7ccccc7c56)nc(-c5cccc6sc7ccccc7c56)n4)cc3o2)cc1 ; c1ccc(-c2nc3cc(-c4cccc(-c5nc(-c6ccc7c(c6)sc6ccccc67)nc(-c6cccc7sc8ccccc8c67)n5)c4)ccc3o2)cc1 ; c1ccc(-c2nc3c(-c4cccc(-c5cccc(-c6nc(-c7ccc8c(c7)sc7ccccc78)nc(-c7ccc8c(c7)sc7ccccc78)n6)c5)c4)cccc3o2)cc1]

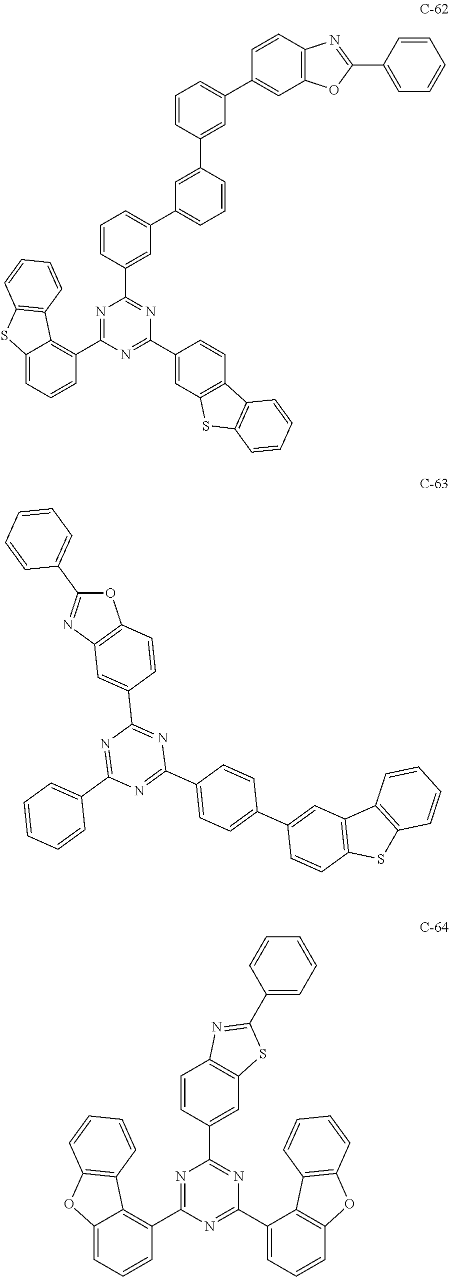 [SMILES: c1ccc(-c2nc3ccc(-c4cccc(-c5cccc(-c6cccc(-c7nc(-c8ccc9c(c8)sc8ccccc89)nc(-c8cccc9sc%10ccccc%10c89)n7)c6)c5)c4)cc3o2)cc1 ; c1ccc(-c2nc(-c3ccc(-c4ccc5sc6ccccc6c5c4)cc3)nc(-c3ccc4oc(-c5ccccc5)nc4c3)n2)cc1 ; c1ccc(-c2nc3ccc(-c4nc(-c5cccc6oc7ccccc7c56)nc(-c5cccc6oc7ccccc7c56)n4)cc3s2)cc1]

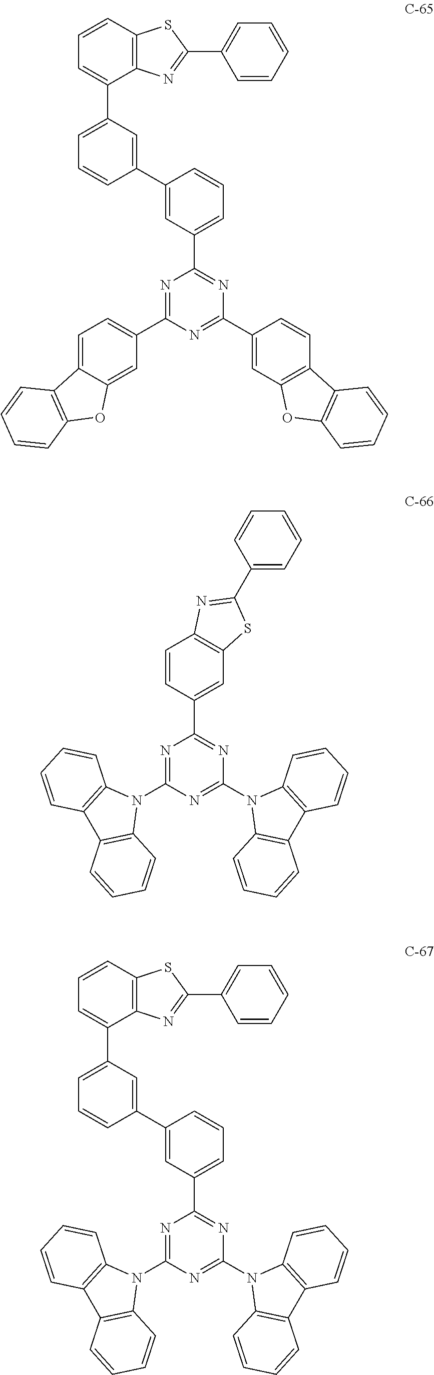 [SMILES: c1ccc(-c2nc3c(-c4cccc(-c5cccc(-c6nc(-c7ccc8c(c7)oc7ccccc78)nc(-c7ccc8c(c7)oc7ccccc78)n6)c5)c4)cccc3s2)cc1 ; c1ccc(-c2nc3ccc(-c4nc(-n5c6ccccc6c6ccccc65)nc(-n5c6ccccc6c6ccccc65)n4)cc3s2)cc1 ; c1ccc(-c2nc3c(-c4cccc(-c5cccc(-c6nc(-n7c8ccccc8c8ccccc87)nc(-n7c8ccccc8c8ccccc87)n6)c5)c4)cccc3s2)cc1]

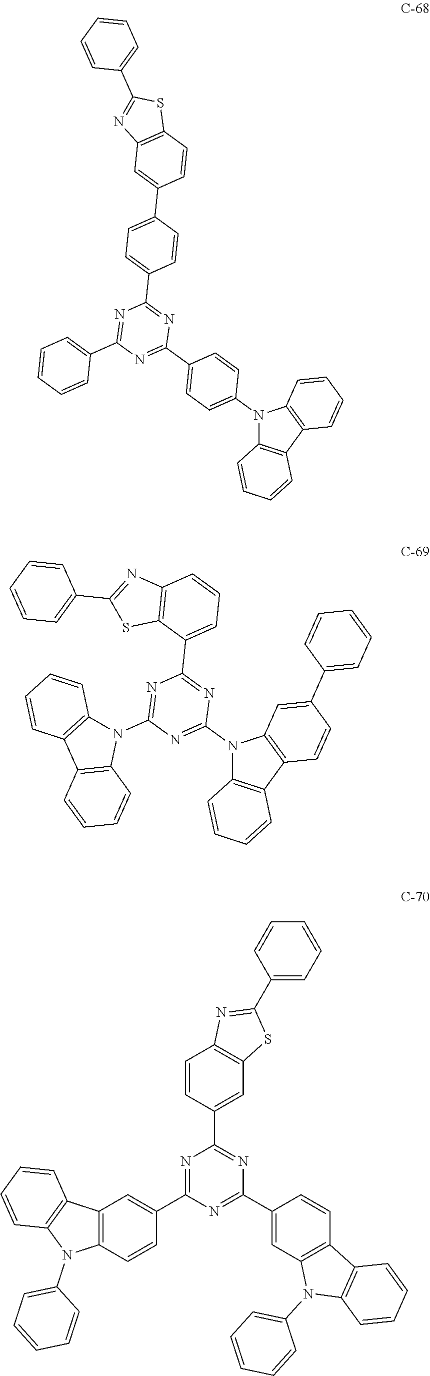 [SMILES: c1ccc(-c2nc(-c3ccc(-c4ccc5sc(-c6ccccc6)nc5c4)cc3)nc(-c3ccc(-n4c5ccccc5c5ccccc54)cc3)n2)cc1 ; c1ccc(-c2ccc3c4ccccc4n(-c4nc(-c5cccc6nc(-c7ccccc7)sc56)nc(-n5c6ccccc6c6ccccc65)n4)c3c2)cc1 ; c1ccc(-c2nc3ccc(-c4nc(-c5ccc6c(c5)c5ccccc5n6-c5ccccc5)nc(-c5ccc6c7ccccc7n(-c7ccccc7)c6c5)n4)cc3s2)cc1]

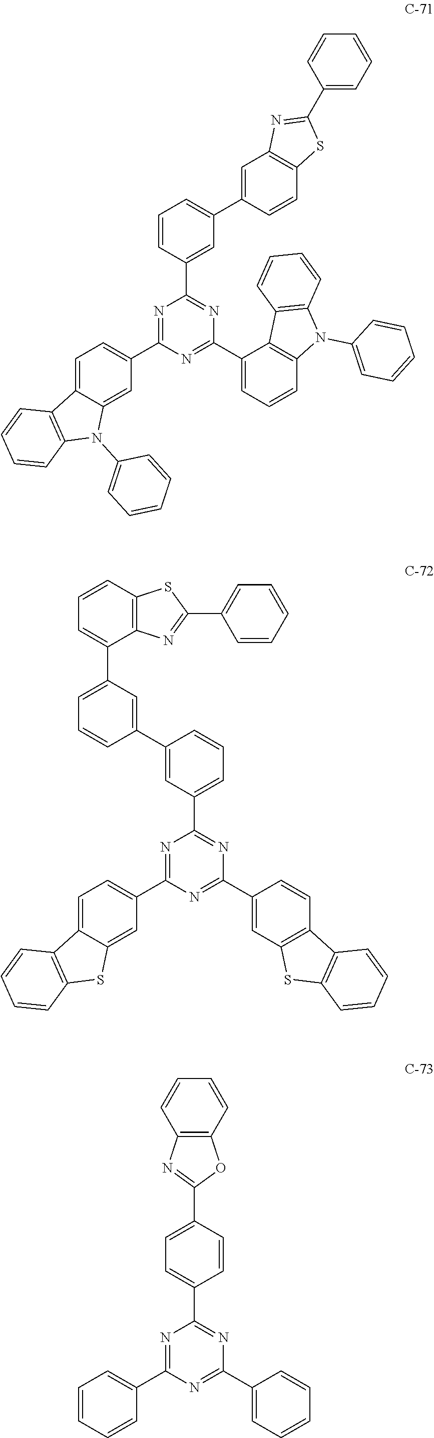 [SMILES: c1ccc(-c2nc3cc(-c4cccc(-c5nc(-c6ccc7c8ccccc8n(-c8ccccc8)c7c6)nc(-c6cccc7c6c6ccccc6n7-c6ccccc6)n5)c4)ccc3s2)cc1 ; c1ccc(-c2nc3c(-c4cccc(-c5cccc(-c6nc(-c7ccc8c(c7)sc7ccccc78)nc(-c7ccc8c(c7)sc7ccccc78)n6)c5)c4)cccc3s2)cc1 ; c1ccc(-c2nc(-c3ccccc3)nc(-c3ccc(-c4nc5ccccc5o4)cc3)n2)cc1]

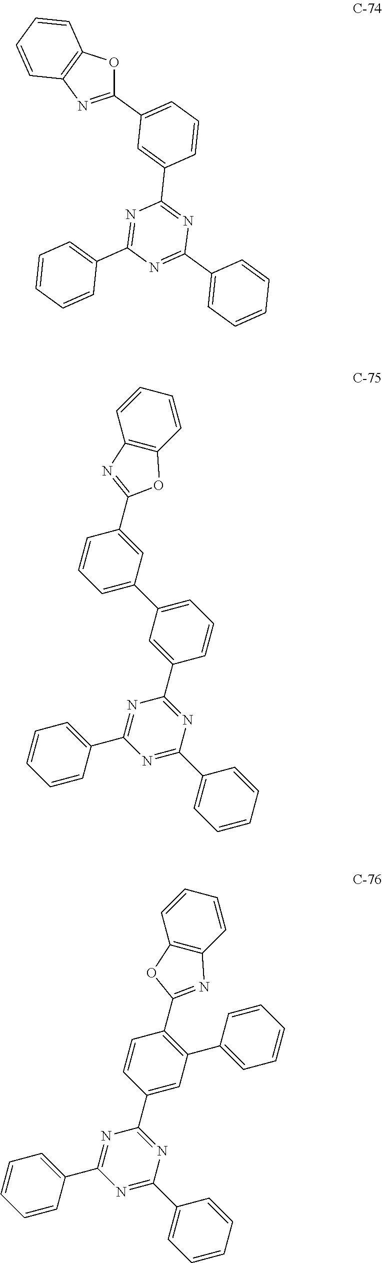 [SMILES: c1ccc(-c2nc(-c3ccccc3)nc(-c3cccc(-c4nc5ccccc5o4)c3)n2)cc1 ; c1ccc(-c2nc(-c3ccccc3)nc(-c3cccc(-c4cccc(-c5nc6ccccc6o5)c4)c3)n2)cc1 ; c1ccc(-c2nc(-c3ccccc3)nc(-c3ccc(-c4nc5ccccc5o4)c(-c4ccccc4)c3)n2)cc1]

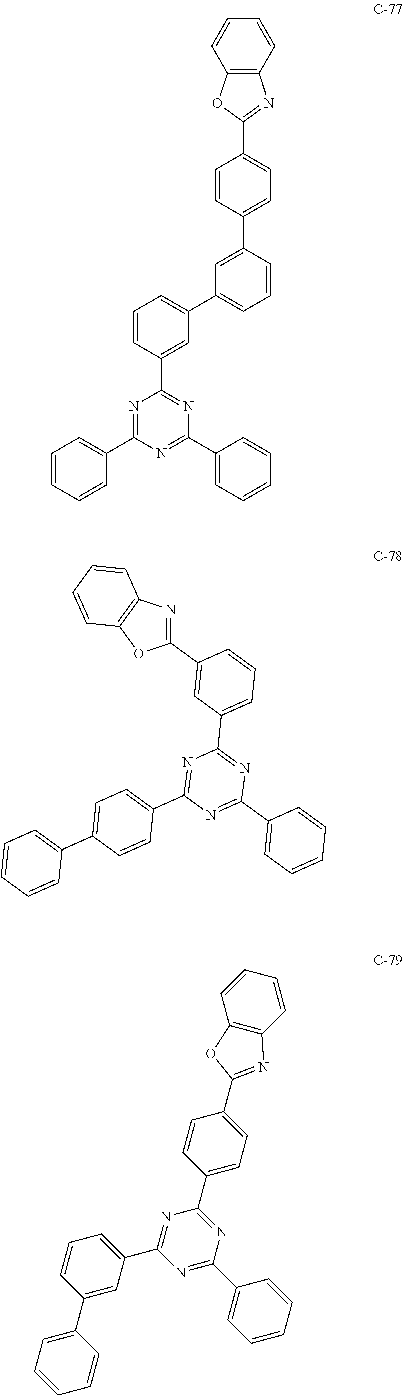 [SMILES: c1ccc(-c2nc(-c3ccccc3)nc(-c3cccc(-c4cccc(-c5ccc(-c6nc7ccccc7o6)cc5)c4)c3)n2)cc1 ; c1ccc(-c2ccc(-c3nc(-c4ccccc4)nc(-c4cccc(-c5nc6ccccc6o5)c4)n3)cc2)cc1 ; c1ccc(-c2cccc(-c3nc(-c4ccccc4)nc(-c4ccc(-c5nc6ccccc6o5)cc4)n3)c2)cc1]

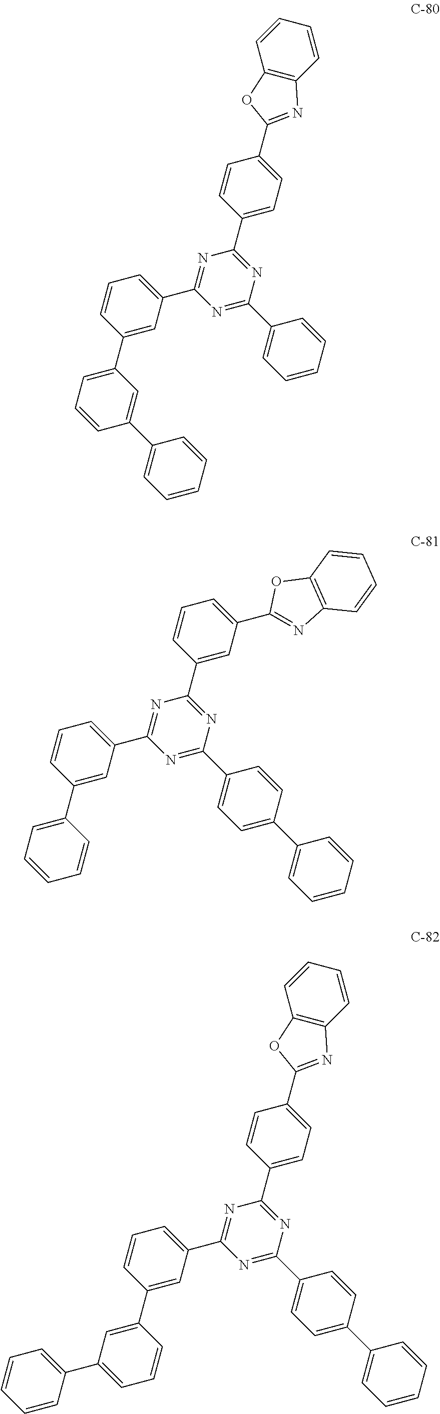 [SMILES: c1ccc(-c2cccc(-c3cccc(-c4nc(-c5ccccc5)nc(-c5ccc(-c6nc7ccccc7o6)cc5)n4)c3)c2)cc1 ; c1ccc(-c2ccc(-c3nc(-c4cccc(-c5ccccc5)c4)nc(-c4cccc(-c5nc6ccccc6o5)c4)n3)cc2)cc1 ; c1ccc(-c2ccc(-c3nc(-c4ccc(-c5nc6ccccc6o5)cc4)nc(-c4cccc(-c5cccc(-c6ccccc6)c5)c4)n3)cc2)cc1]

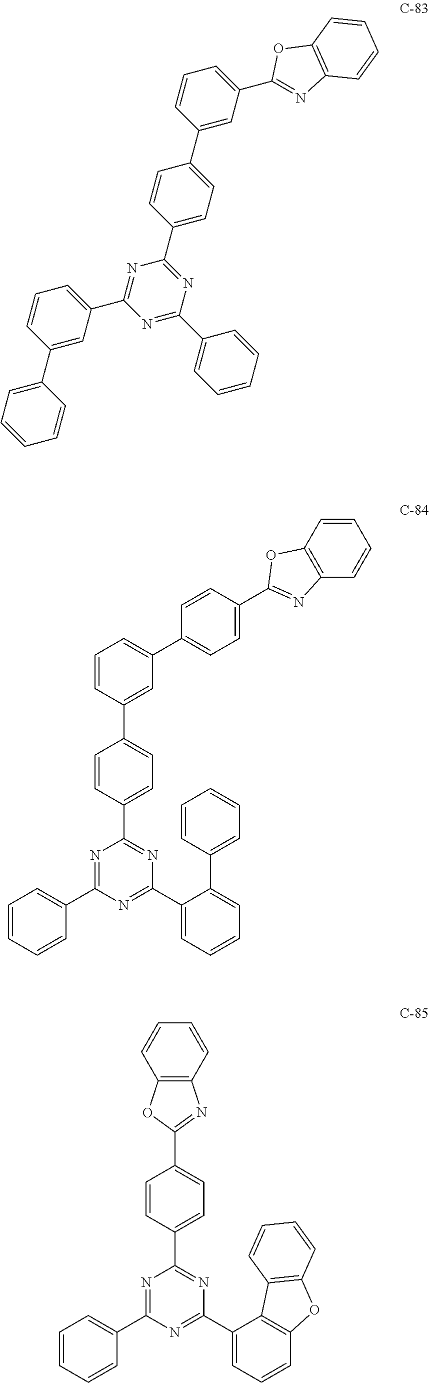 [SMILES: c1ccc(-c2cccc(-c3nc(-c4ccccc4)nc(-c4ccc(-c5cccc(-c6nc7ccccc7o6)c5)cc4)n3)c2)cc1 ; c1ccc(-c2nc(-c3ccc(-c4cccc(-c5ccc(-c6nc7ccccc7o6)cc5)c4)cc3)nc(-c3ccccc3-c3ccccc3)n2)cc1 ; c1ccc(-c2nc(-c3ccc(-c4nc5ccccc5o4)cc3)nc(-c3cccc4oc5ccccc5c34)n2)cc1]

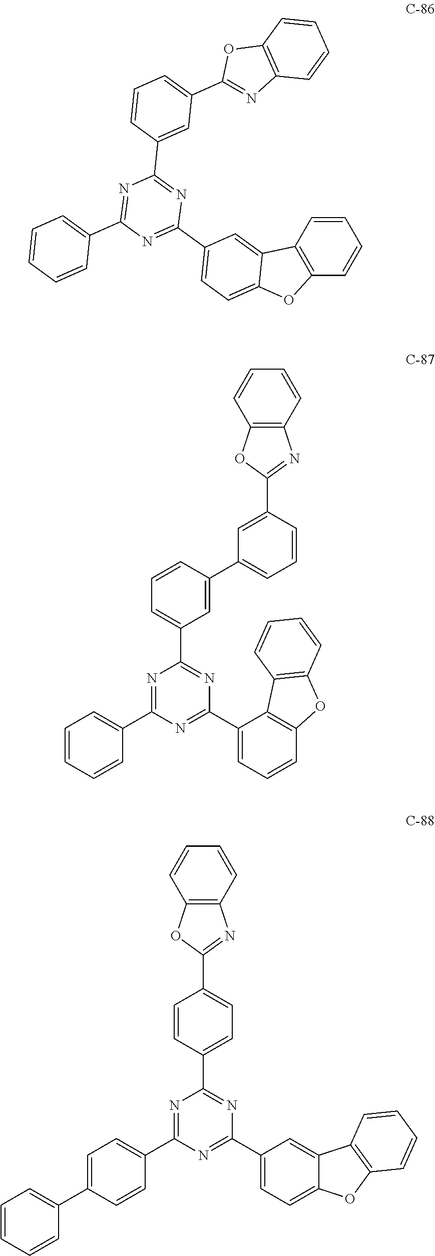 [SMILES: c1ccc(-c2nc(-c3cccc(-c4nc5ccccc5o4)c3)nc(-c3ccc4oc5ccccc5c4c3)n2)cc1 ; c1ccc(-c2nc(-c3cccc(-c4cccc(-c5nc6ccccc6o5)c4)c3)nc(-c3cccc4oc5ccccc5c34)n2)cc1 ; c1ccc(-c2ccc(-c3nc(-c4ccc(-c5nc6ccccc6o5)cc4)nc(-c4ccc5oc6ccccc6c5c4)n3)cc2)cc1]

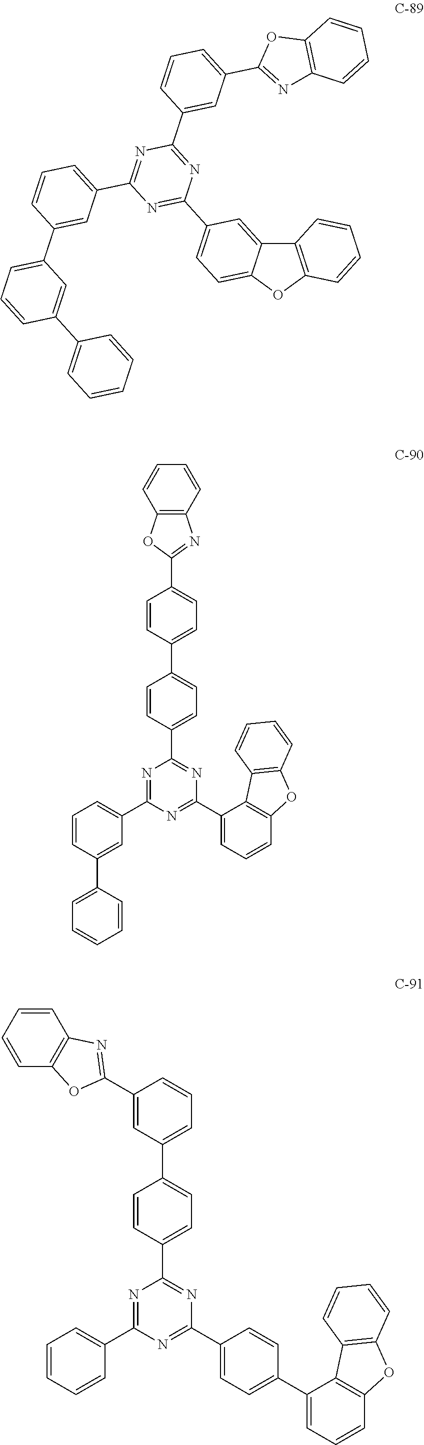 [SMILES: c1ccc(-c2cccc(-c3cccc(-c4nc(-c5cccc(-c6nc7ccccc7o6)c5)nc(-c5ccc6oc7ccccc7c6c5)n4)c3)c2)cc1 ; c1ccc(-c2cccc(-c3nc(-c4ccc(-c5ccc(-c6nc7ccccc7o6)cc5)cc4)nc(-c4cccc5oc6ccccc6c45)n3)c2)cc1 ; c1ccc(-c2nc(-c3ccc(-c4cccc(-c5nc6ccccc6o5)c4)cc3)nc(-c3ccc(-c4cccc5oc6ccccc6c45)cc3)n2)cc1]

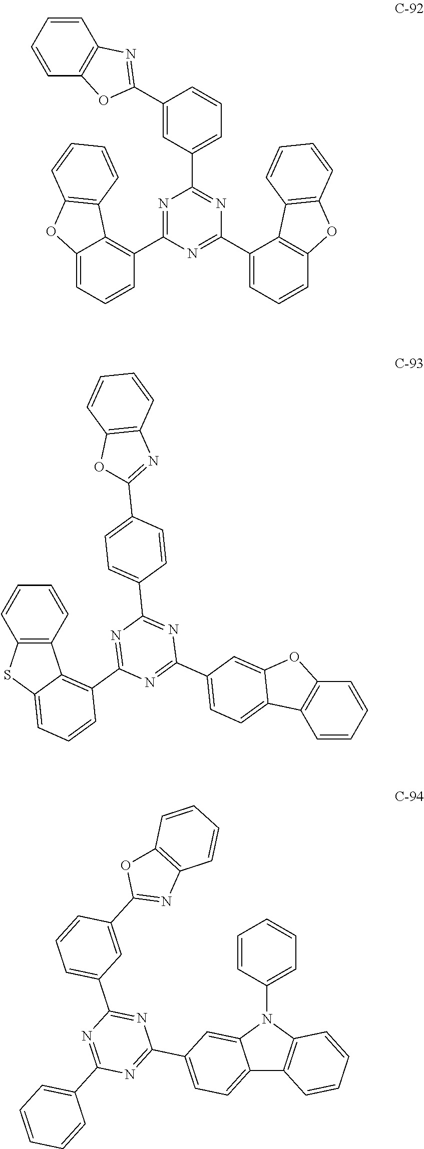 [SMILES: c1cc(-c2nc(-c3cccc4oc5ccccc5c34)nc(-c3cccc4oc5ccccc5c34)n2)cc(-c2nc3ccccc3o2)c1 ; c1ccc2oc(-c3ccc(-c4nc(-c5ccc6c(c5)oc5ccccc56)nc(-c5cccc6sc7ccccc7c56)n4)cc3)nc2c1 ; c1ccc(-c2nc(-c3cccc(-c4nc5ccccc5o4)c3)nc(-c3ccc4c5ccccc5n(-c5ccccc5)c4c3)n2)cc1]

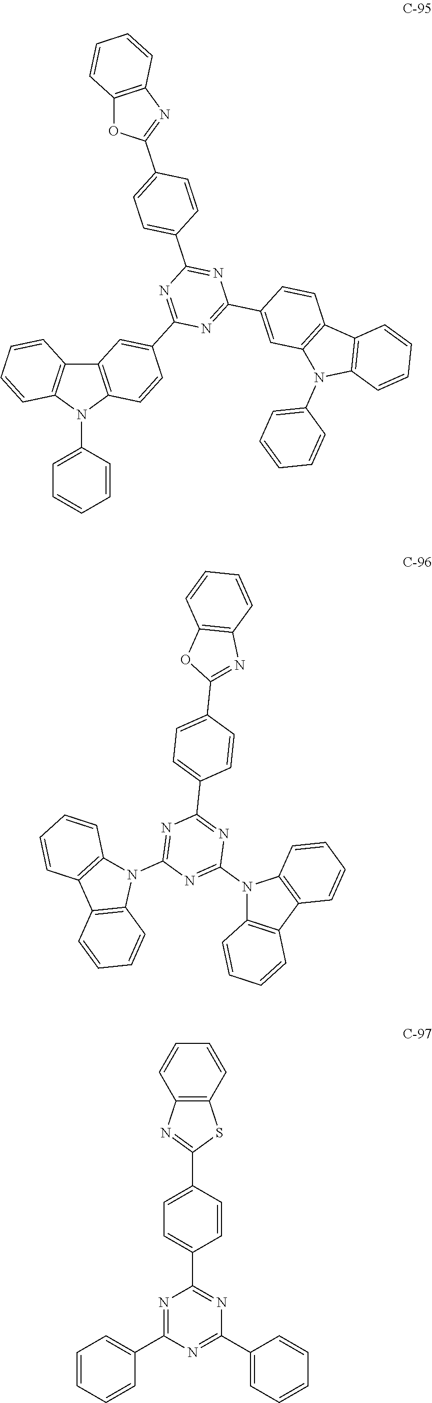 [SMILES: c1ccc(-n2c3ccccc3c3cc(-c4nc(-c5ccc(-c6nc7ccccc7o6)cc5)nc(-c5ccc6c7ccccc7n(-c7ccccc7)c6c5)n4)ccc32)cc1 ; c1ccc2oc(-c3ccc(-c4nc(-n5c6ccccc6c6ccccc65)nc(-n5c6ccccc6c6ccccc65)n4)cc3)nc2c1 ; c1ccc(-c2nc(-c3ccccc3)nc(-c3ccc(-c4nc5ccccc5s4)cc3)n2)cc1]

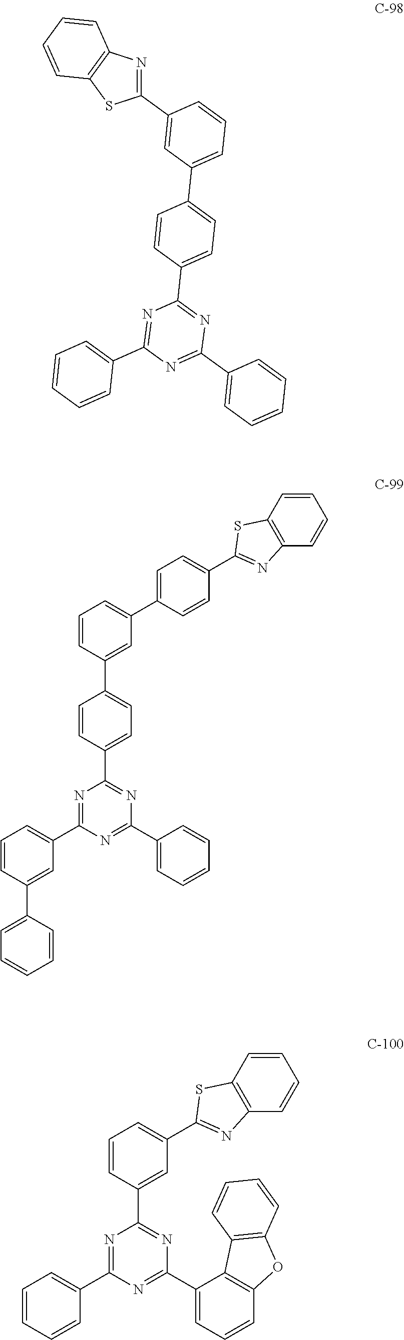 [SMILES: c1ccc(-c2nc(-c3ccccc3)nc(-c3ccc(-c4cccc(-c5nc6ccccc6s5)c4)cc3)n2)cc1 ; c1ccc(-c2cccc(-c3nc(-c4ccccc4)nc(-c4ccc(-c5cccc(-c6ccc(-c7nc8ccccc8s7)cc6)c5)cc4)n3)c2)cc1 ; c1ccc(-c2nc(-c3cccc(-c4nc5ccccc5s4)c3)nc(-c3cccc4oc5ccccc5c34)n2)cc1]

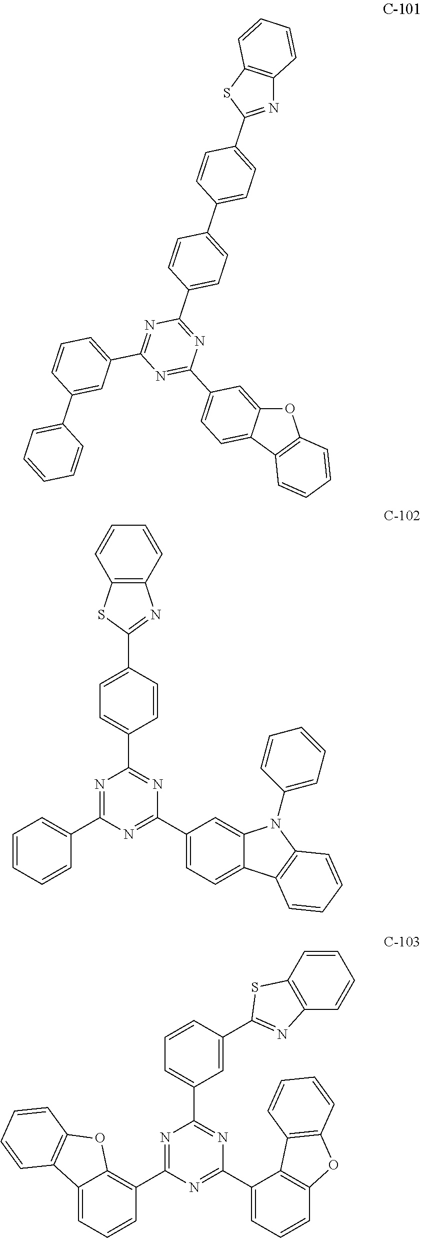

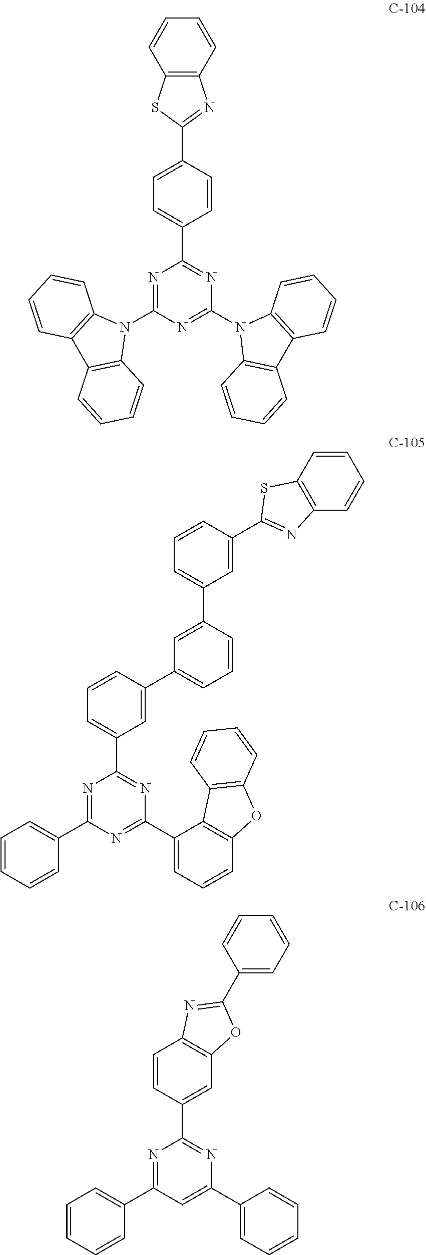

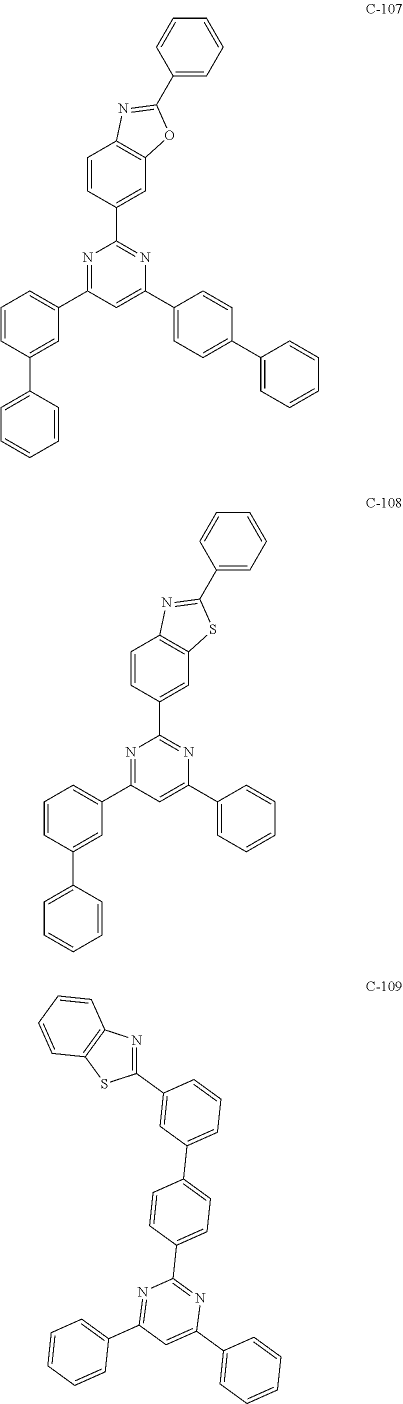

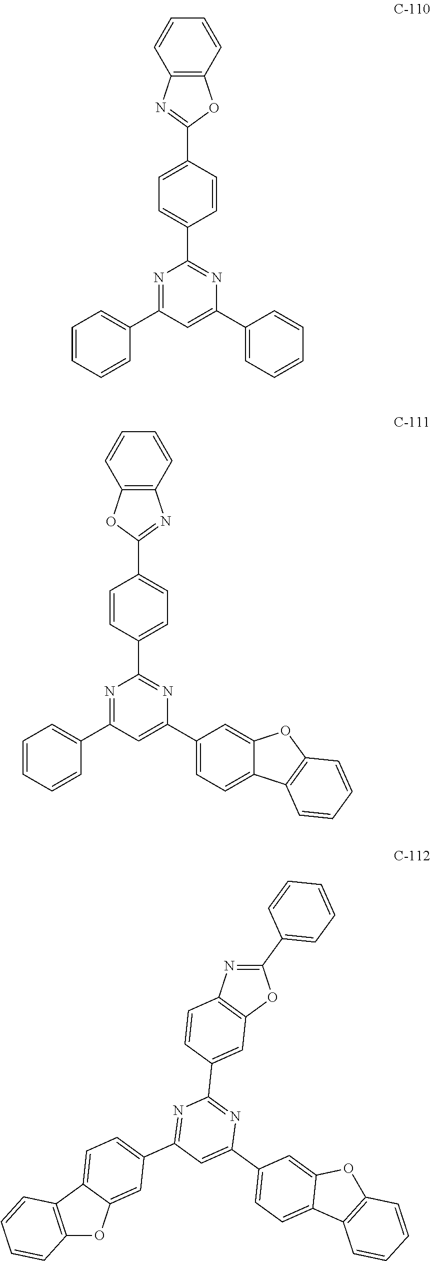 [SMILES: c1ccc(-c2cc(-c3ccccc3)nc(-c3ccc(-c4nc5ccccc5o4)cc3)n2)cc1 ; c1ccc(-c2cc(-c3ccc4c(c3)oc3ccccc34)nc(-c3ccc(-c4nc5ccccc5o4)cc3)n2)cc1 ; c1ccc(-c2nc3ccc(-c4nc(-c5ccc6c(c5)oc5ccccc56)cc(-c5ccc6c(c5)oc5ccccc56)n4)cc3o2)cc1]

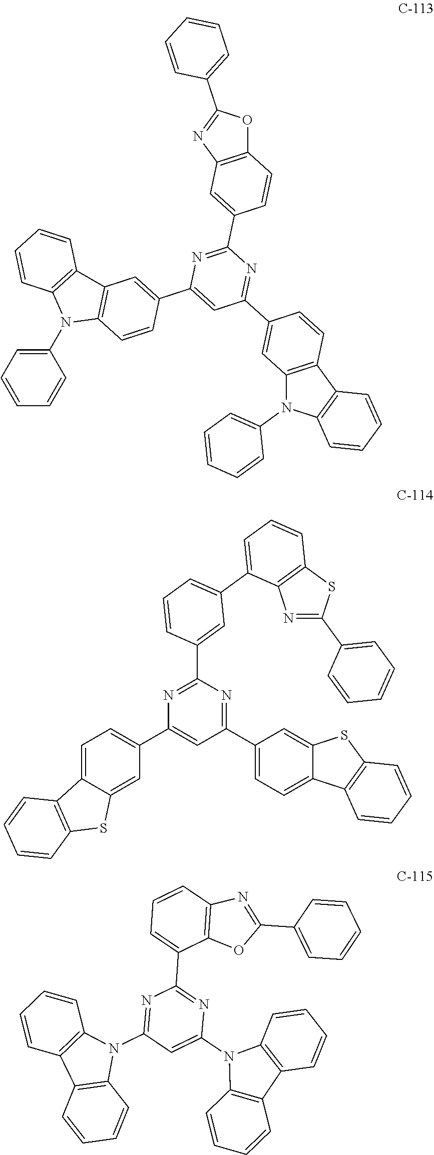 [SMILES: c1ccc(-c2nc3cc(-c4nc(-c5ccc6c(c5)c5ccccc5n6-c5ccccc5)cc(-c5ccc6c7ccccc7n(-c7ccccc7)c6c5)n4)ccc3o2)cc1 ; c1ccc(-c2nc3c(-c4cccc(-c5nc(-c6ccc7c(c6)sc6ccccc67)cc(-c6ccc7c(c6)sc6ccccc67)n5)c4)cccc3s2)cc1 ; c1ccc(-c2nc3cccc(-c4nc(-n5c6ccccc6c6ccccc65)cc(-n5c6ccccc6c6ccccc65)n4)c3o2)cc1]

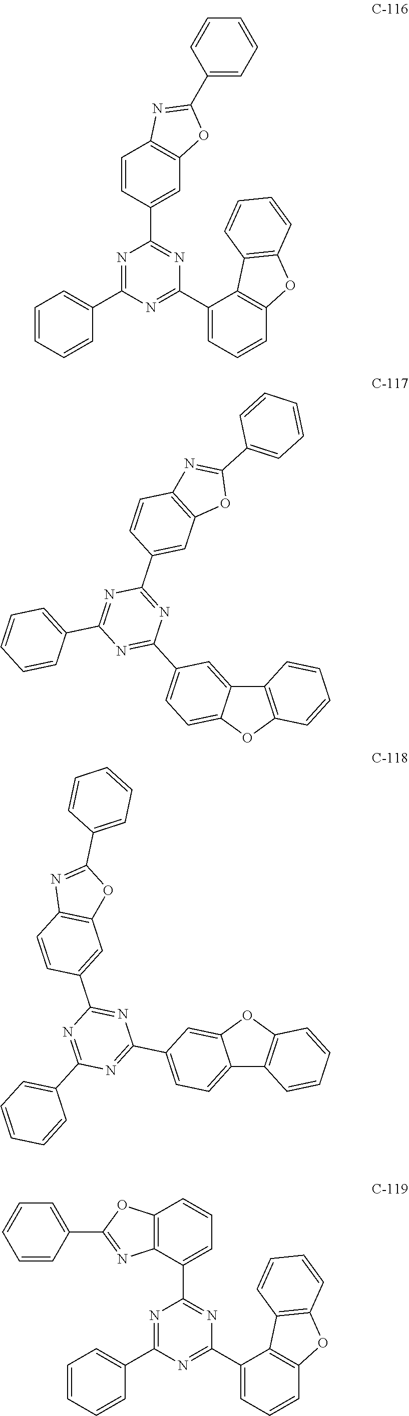

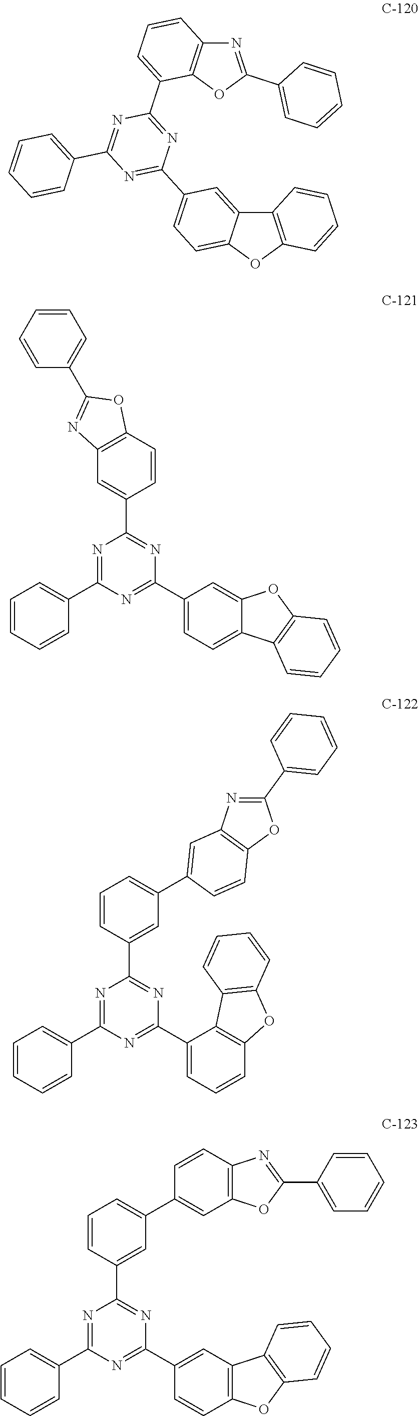

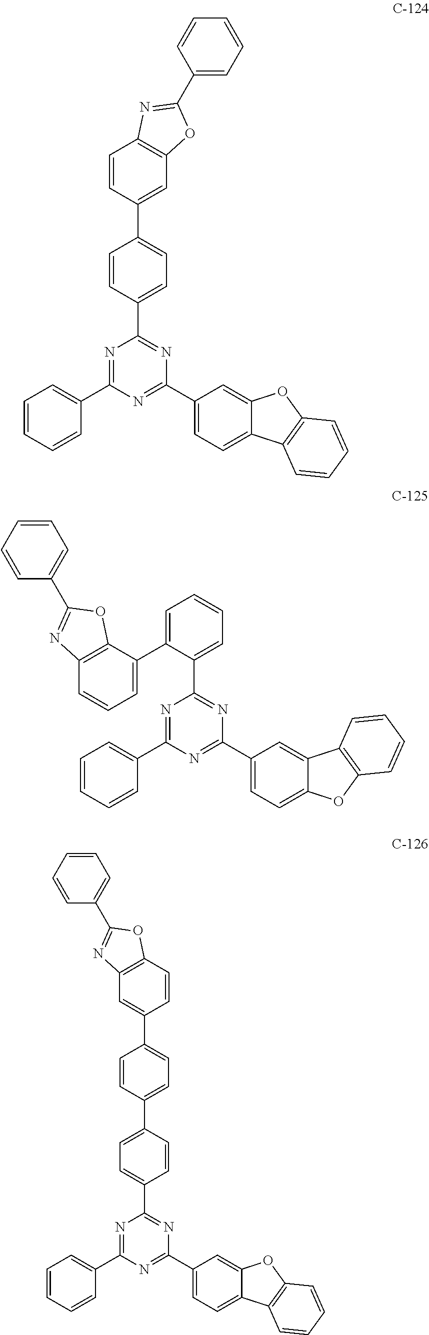 [SMILES: c1ccc(-c2nc(-c3ccc(-c4ccc5nc(-c6ccccc6)oc5c4)cc3)nc(-c3ccc4c(c3)oc3ccccc34)n2)cc1 ; c1ccc(-c2nc(-c3ccc4oc5ccccc5c4c3)nc(-c3ccccc3-c3cccc4nc(-c5ccccc5)oc34)n2)cc1 ; c1ccc(-c2nc(-c3ccc(-c4ccc(-c5ccc6oc(-c7ccccc7)nc6c5)cc4)cc3)nc(-c3ccc4c(c3)oc3ccccc34)n2)cc1]

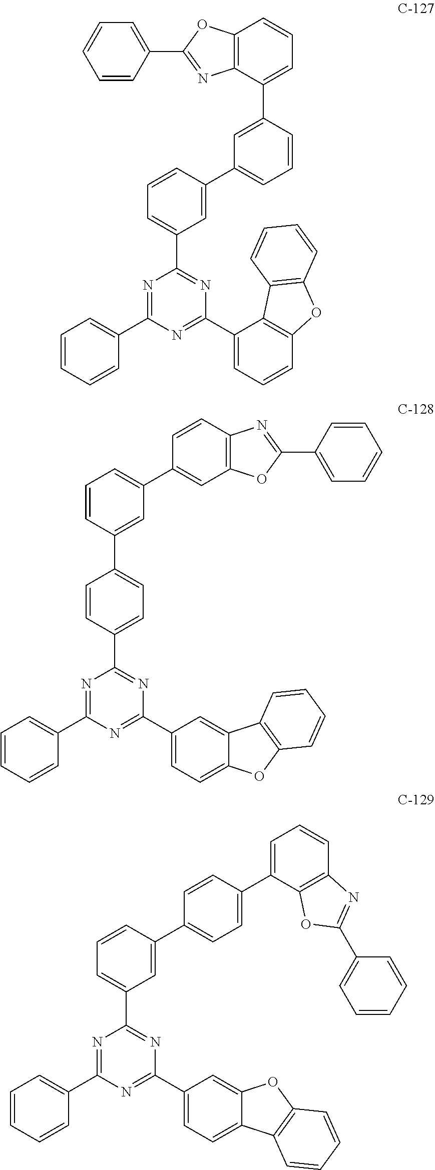

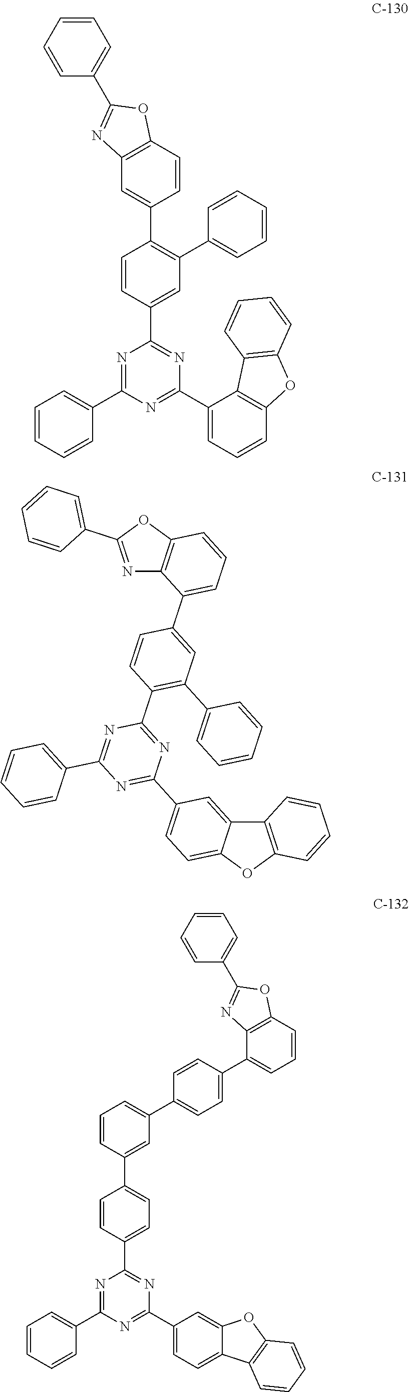

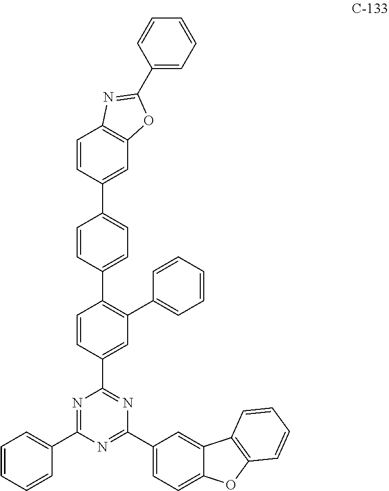 [SMILES: c1ccc(-c2nc(-c3ccc(-c4ccc(-c5ccc6nc(-c7ccccc7)oc6c5)cc4)c(-c4ccccc4)c3)nc(-c3ccc4oc5ccccc5c4c3)n2)cc1]

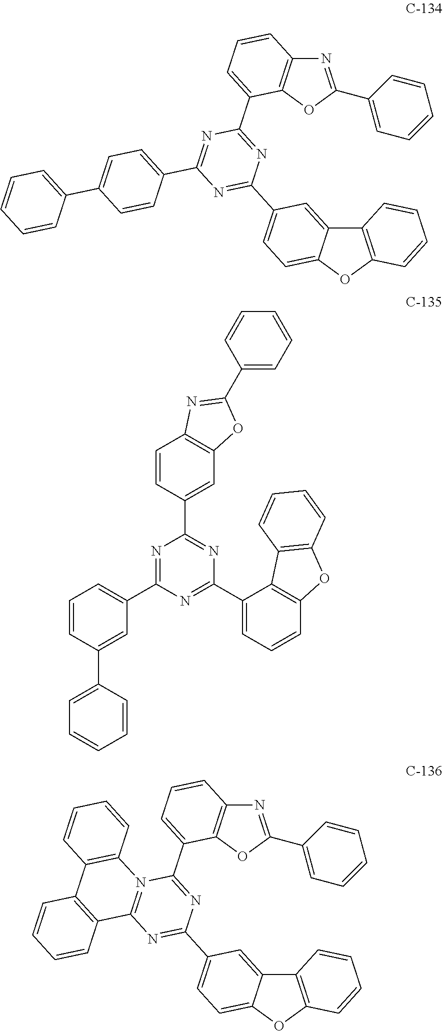

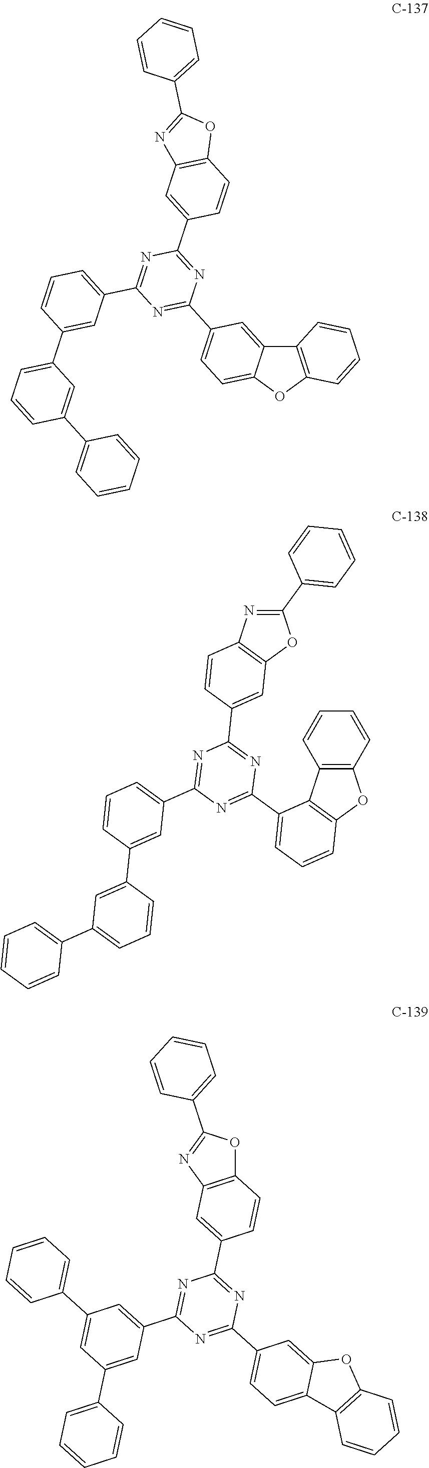 [SMILES: c1ccc(-c2cccc(-c3cccc(-c4nc(-c5ccc6oc(-c7ccccc7)nc6c5)nc(-c5ccc6oc7ccccc7c6c5)n4)c3)c2)cc1 ; c1ccc(-c2cccc(-c3cccc(-c4nc(-c5ccc6nc(-c7ccccc7)oc6c5)nc(-c5cccc6oc7ccccc7c56)n4)c3)c2)cc1 ; c1ccc(-c2cc(-c3ccccc3)cc(-c3nc(-c4ccc5oc(-c6ccccc6)nc5c4)nc(-c4ccc5c(c4)oc4ccccc45)n3)c2)cc1]

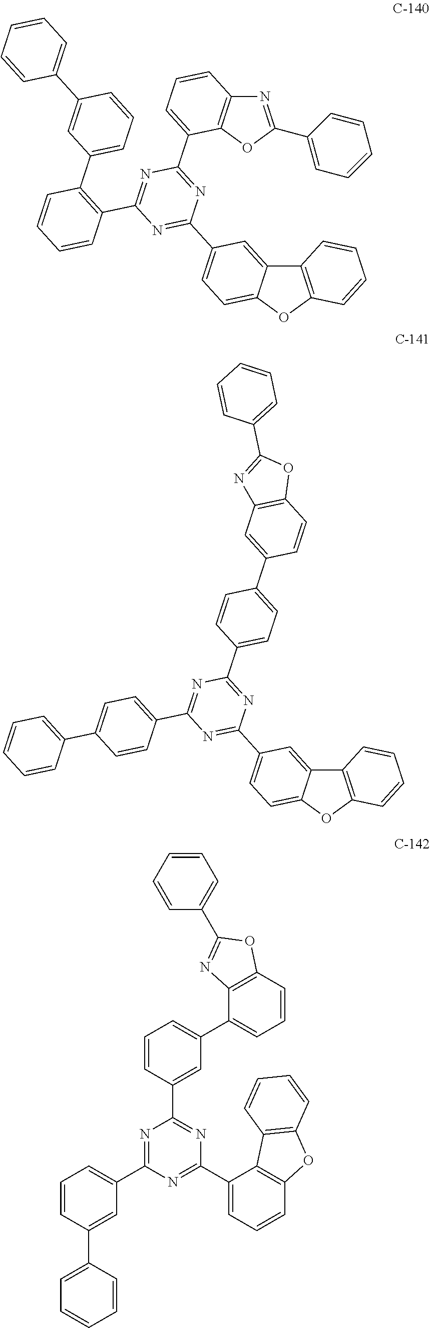 [SMILES: c1ccc(-c2cccc(-c3ccccc3-c3nc(-c4ccc5oc6ccccc6c5c4)nc(-c4cccc5nc(-c6ccccc6)oc45)n3)c2)cc1 ; c1ccc(-c2ccc(-c3nc(-c4ccc(-c5ccc6oc(-c7ccccc7)nc6c5)cc4)nc(-c4ccc5oc6ccccc6c5c4)n3)cc2)cc1 ; c1ccc(-c2cccc(-c3nc(-c4cccc(-c5cccc6oc(-c7ccccc7)nc56)c4)nc(-c4cccc5oc6ccccc6c45)n3)c2)cc1]

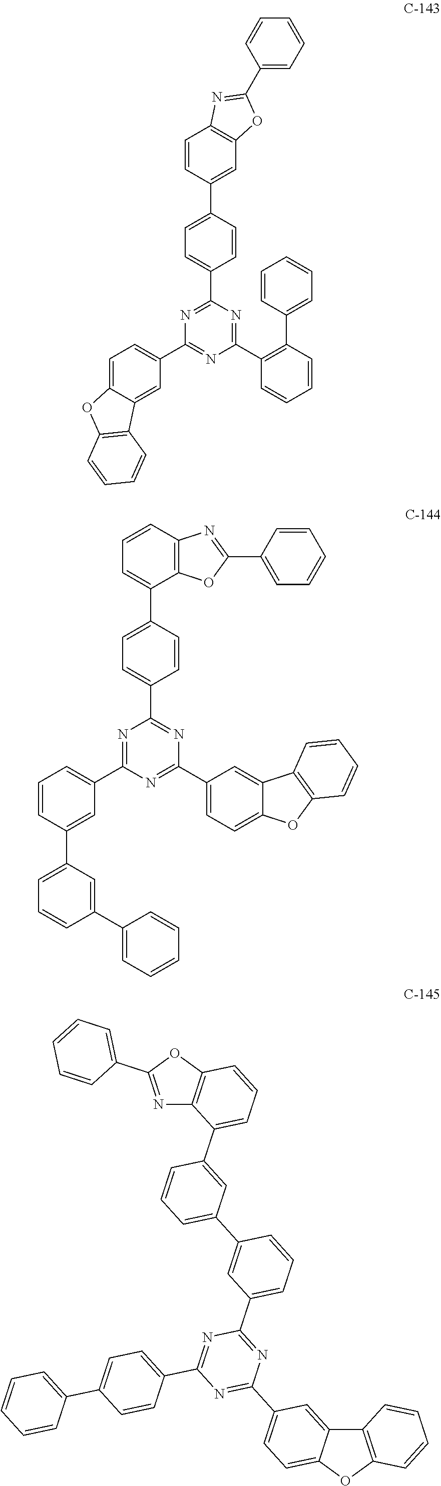 [SMILES: c1ccc(-c2nc3ccc(-c4ccc(-c5nc(-c6ccc7oc8ccccc8c7c6)nc(-c6ccccc6-c6ccccc6)n5)cc4)cc3o2)cc1 ; c1ccc(-c2cccc(-c3cccc(-c4nc(-c5ccc(-c6cccc7nc(-c8ccccc8)oc67)cc5)nc(-c5ccc6oc7ccccc7c6c5)n4)c3)c2)cc1 ; c1ccc(-c2ccc(-c3nc(-c4cccc(-c5cccc(-c6cccc7oc(-c8ccccc8)nc67)c5)c4)nc(-c4ccc5oc6ccccc6c5c4)n3)cc2)cc1]

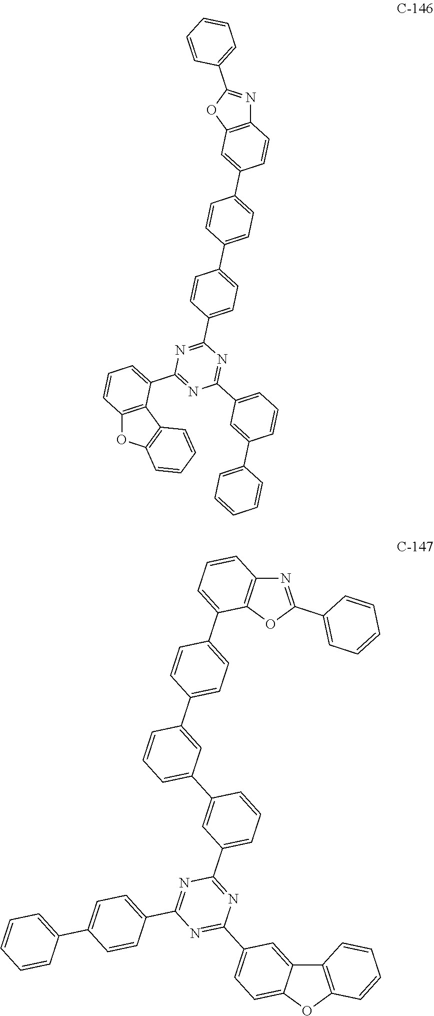

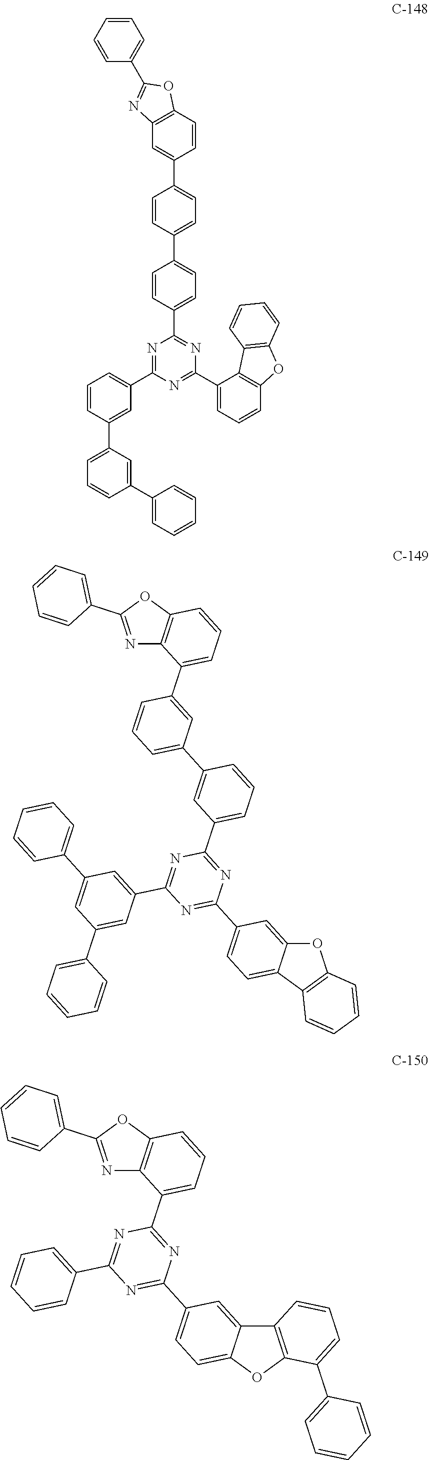 [SMILES: c1ccc(-c2cccc(-c3cccc(-c4nc(-c5ccc(-c6ccc(-c7ccc8oc(-c9ccccc9)nc8c7)cc6)cc5)nc(-c5cccc6oc7ccccc7c56)n4)c3)c2)cc1 ; c1ccc(-c2cc(-c3ccccc3)cc(-c3nc(-c4cccc(-c5cccc(-c6cccc7oc(-c8ccccc8)nc67)c5)c4)nc(-c4ccc5c(c4)oc4ccccc45)n3)c2)cc1 ; c1ccc(-c2nc(-c3ccc4oc5c(-c6ccccc6)cccc5c4c3)nc(-c3cccc4oc(-c5ccccc5)nc34)n2)cc1]

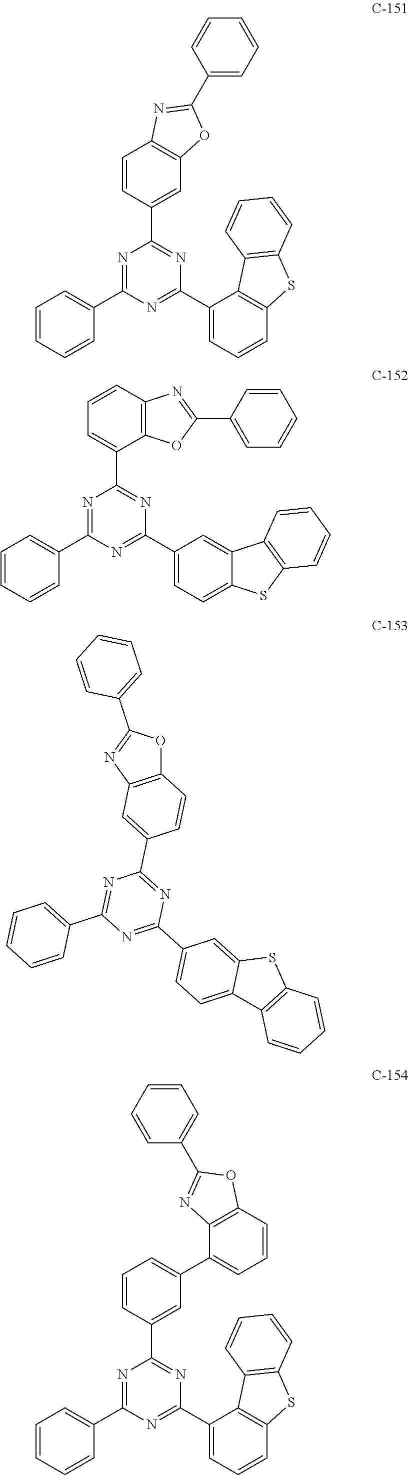

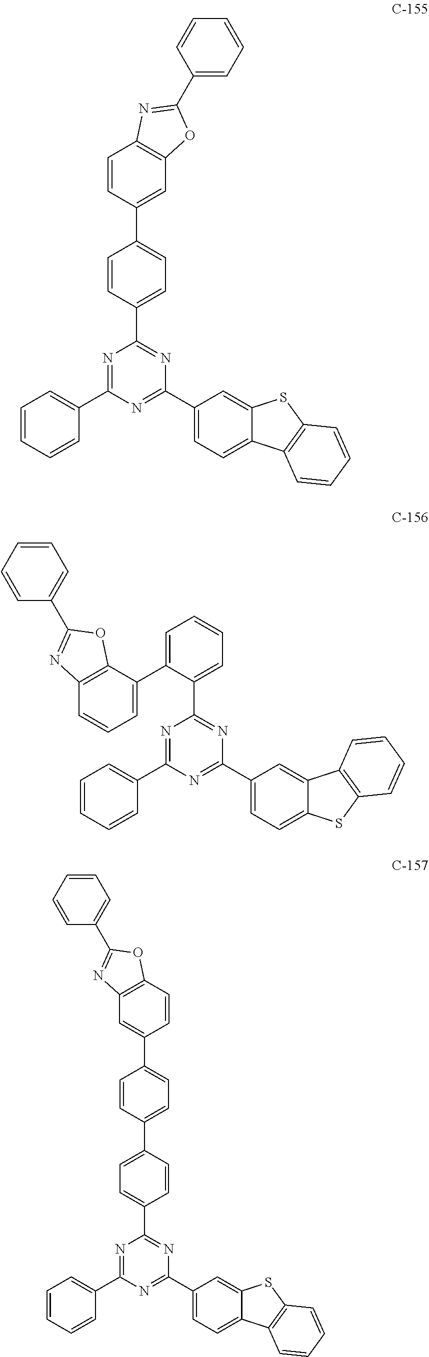 [SMILES: c1ccc(-c2nc(-c3ccc(-c4ccc5nc(-c6ccccc6)oc5c4)cc3)nc(-c3ccc4c(c3)sc3ccccc34)n2)cc1 ; c1ccc(-c2nc(-c3ccc4sc5ccccc5c4c3)nc(-c3ccccc3-c3cccc4nc(-c5ccccc5)oc34)n2)cc1 ; c1ccc(-c2nc(-c3ccc(-c4ccc(-c5ccc6oc(-c7ccccc7)nc6c5)cc4)cc3)nc(-c3ccc4c(c3)sc3ccccc34)n2)cc1]

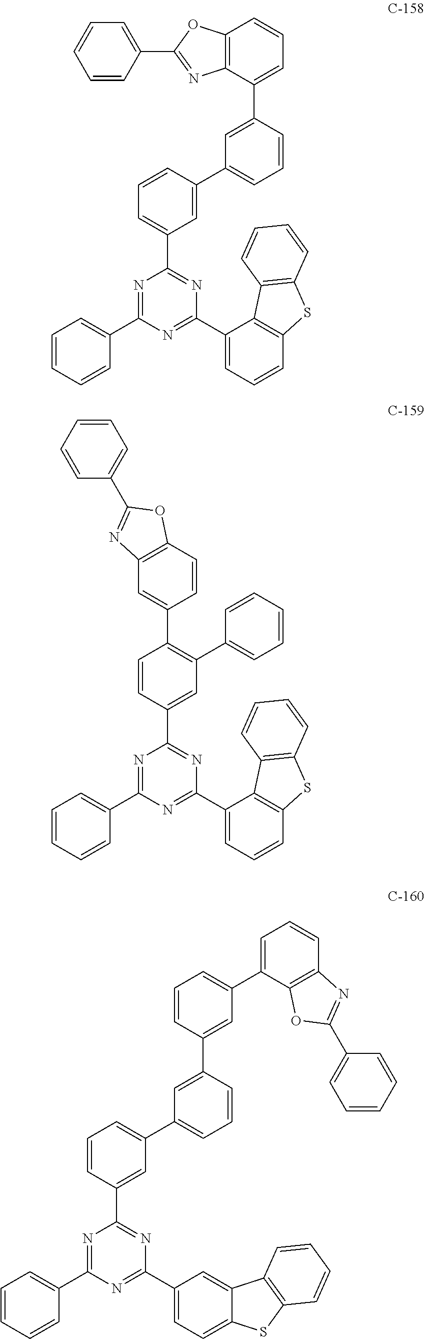

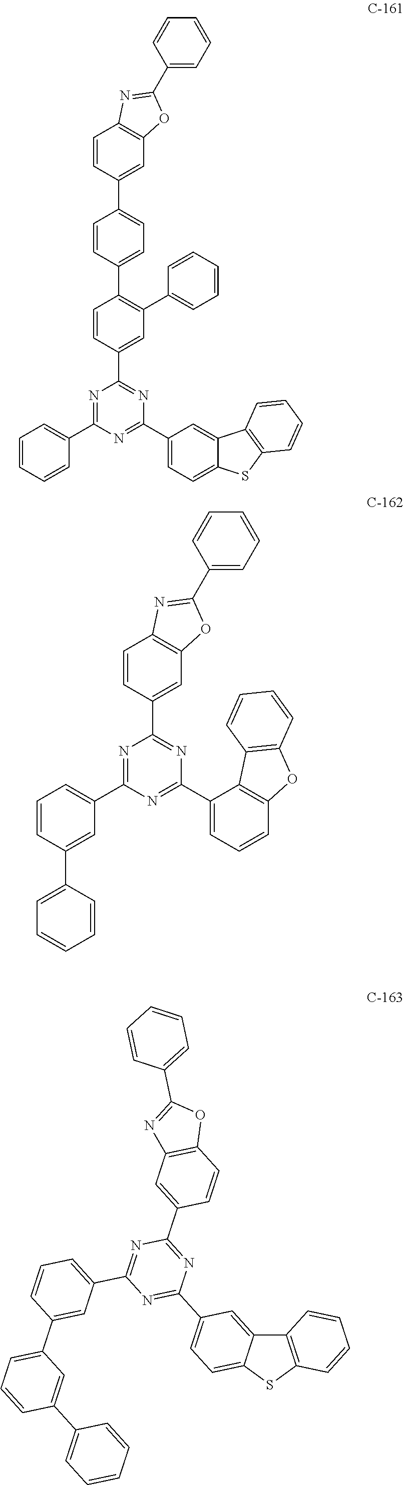

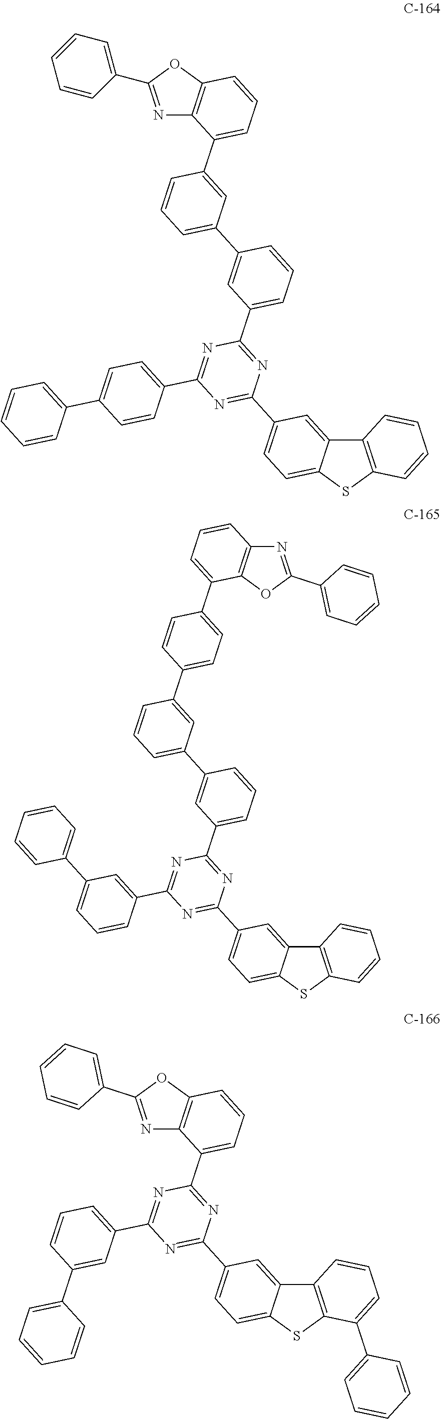 [SMILES: c1ccc(-c2ccc(-c3nc(-c4cccc(-c5cccc(-c6cccc7oc(-c8ccccc8)nc67)c5)c4)nc(-c4ccc5sc6ccccc6c5c4)n3)cc2)cc1 ; c1ccc(-c2cccc(-c3nc(-c4cccc(-c5cccc(-c6ccc(-c7cccc8nc(-c9ccccc9)oc78)cc6)c5)c4)nc(-c4ccc5sc6ccccc6c5c4)n3)c2)cc1 ; c1ccc(-c2cccc(-c3nc(-c4ccc5sc6c(-c7ccccc7)cccc6c5c4)nc(-c4cccc5oc(-c6ccccc6)nc45)n3)c2)cc1]

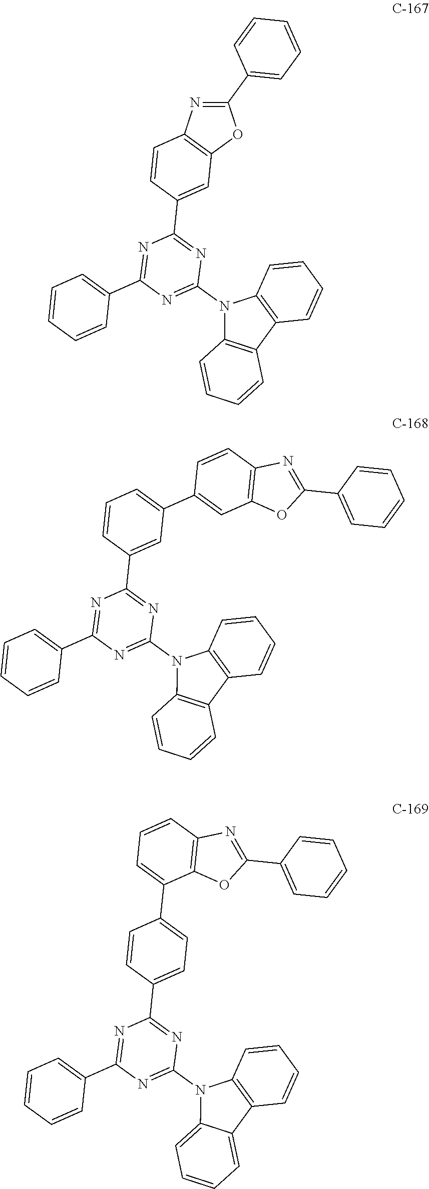

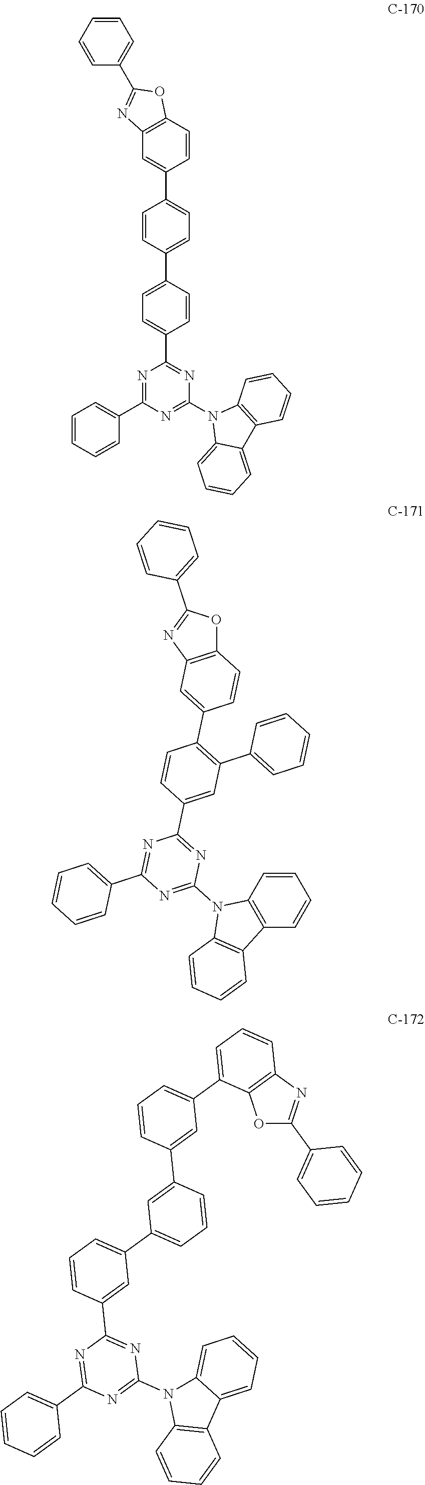

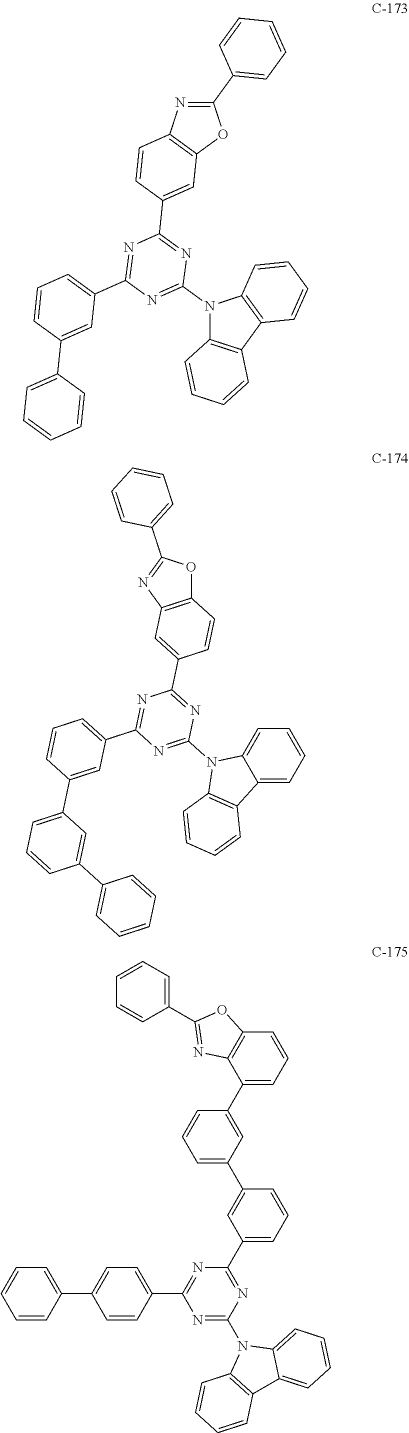

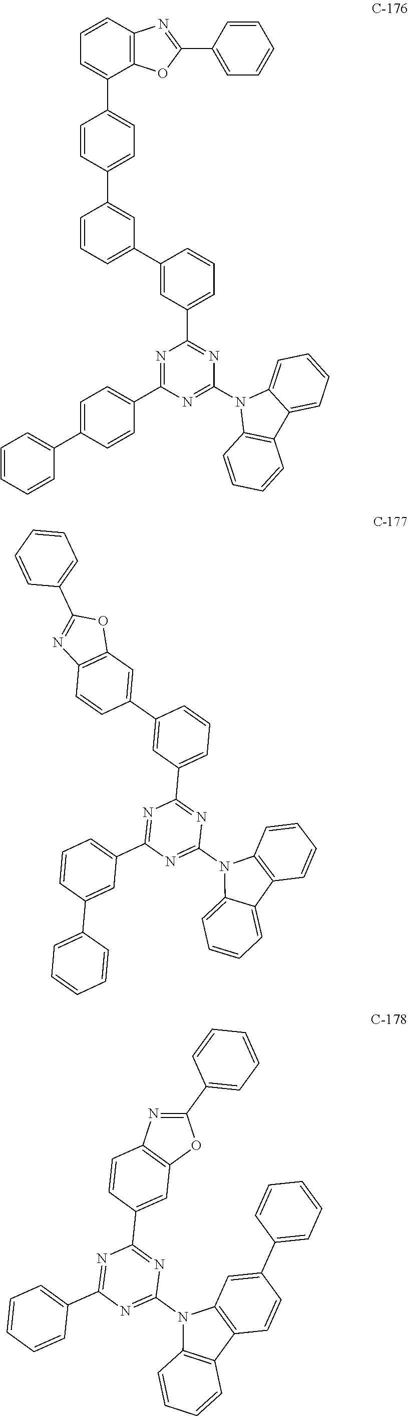

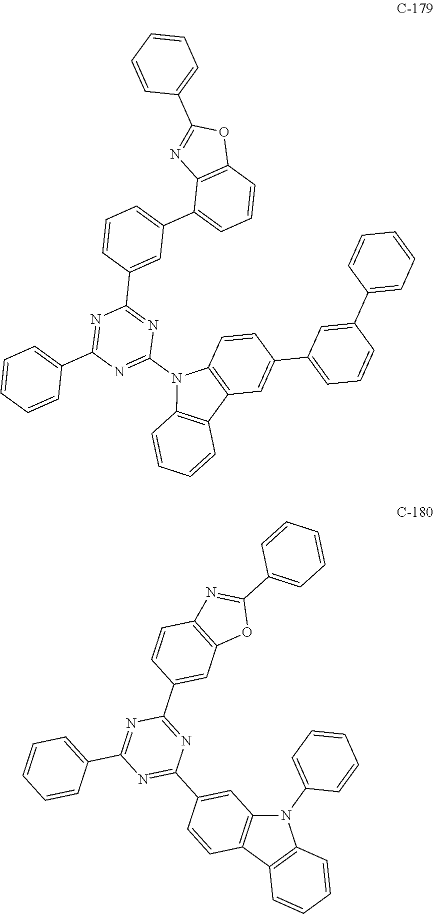 [SMILES: c1ccc(-c2cccc(-c3ccc4c(c3)c3ccccc3n4-c3nc(-c4ccccc4)nc(-c4cccc(-c5cccc6oc(-c7ccccc7)nc56)c4)n3)c2)cc1 ; c1ccc(-c2nc(-c3ccc4nc(-c5ccccc5)oc4c3)nc(-c3ccc4c5ccccc5n(-c5ccccc5)c4c3)n2)cc1]

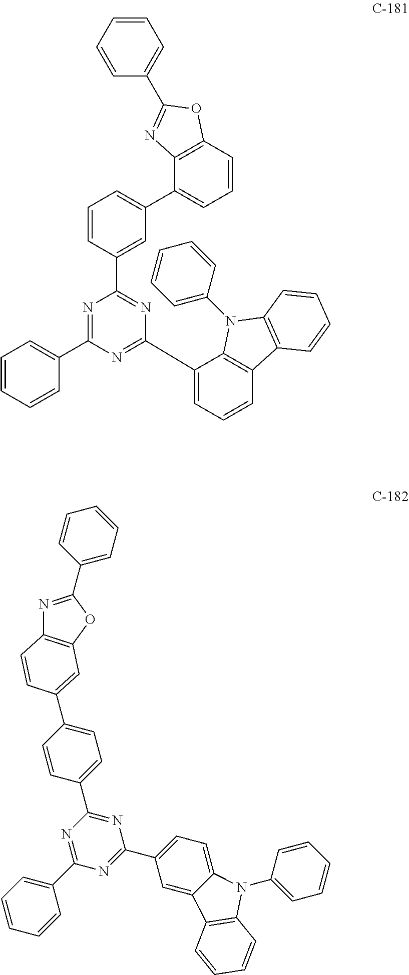 [SMILES: c1ccc(-c2nc(-c3cccc(-c4cccc5oc(-c6ccccc6)nc45)c3)nc(-c3cccc4c5ccccc5n(-c5ccccc5)c34)n2)cc1 ; c1ccc(-c2nc(-c3ccc(-c4ccc5nc(-c6ccccc6)oc5c4)cc3)nc(-c3ccc4c(c3)c3ccccc3n4-c3ccccc3)n2)cc1]

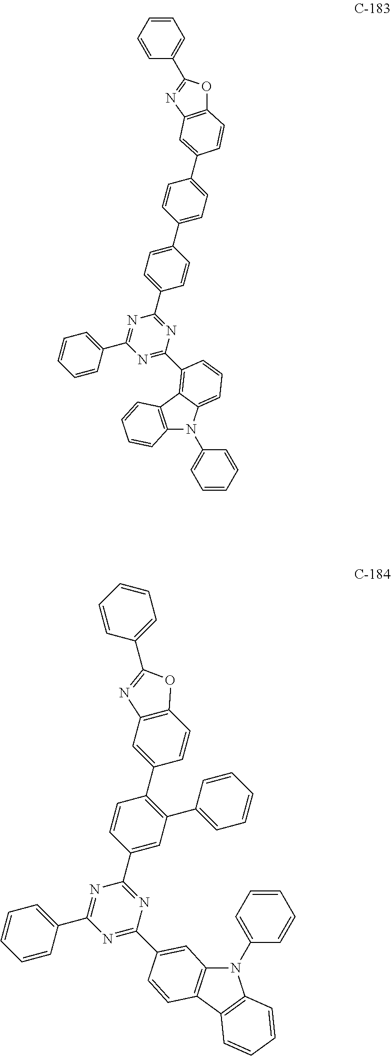 [SMILES: c1ccc(-c2nc(-c3ccc(-c4ccc(-c5ccc6oc(-c7ccccc7)nc6c5)cc4)cc3)nc(-c3cccc4c3c3ccccc3n4-c3ccccc3)n2)cc1 ; c1ccc(-c2nc(-c3ccc(-c4ccc5oc(-c6ccccc6)nc5c4)c(-c4ccccc4)c3)nc(-c3ccc4c5ccccc5n(-c5ccccc5)c4c3)n2)cc1]

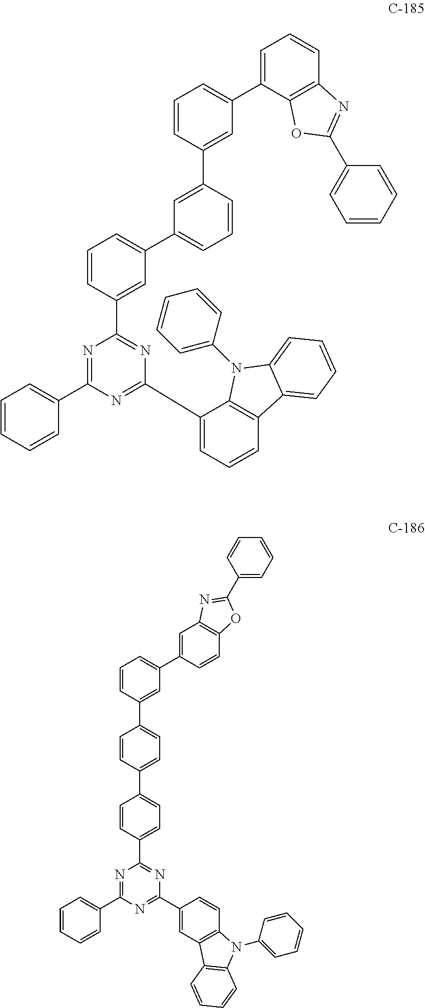 [SMILES: c1ccc(-c2nc(-c3cccc(-c4cccc(-c5cccc(-c6cccc7nc(-c8ccccc8)oc67)c5)c4)c3)nc(-c3cccc4c5ccccc5n(-c5ccccc5)c34)n2)cc1 ; c1ccc(-c2nc(-c3ccc(-c4ccc(-c5cccc(-c6ccc7oc(-c8ccccc8)nc7c6)c5)cc4)cc3)nc(-c3ccc4c(c3)c3ccccc3n4-c3ccccc3)n2)cc1]

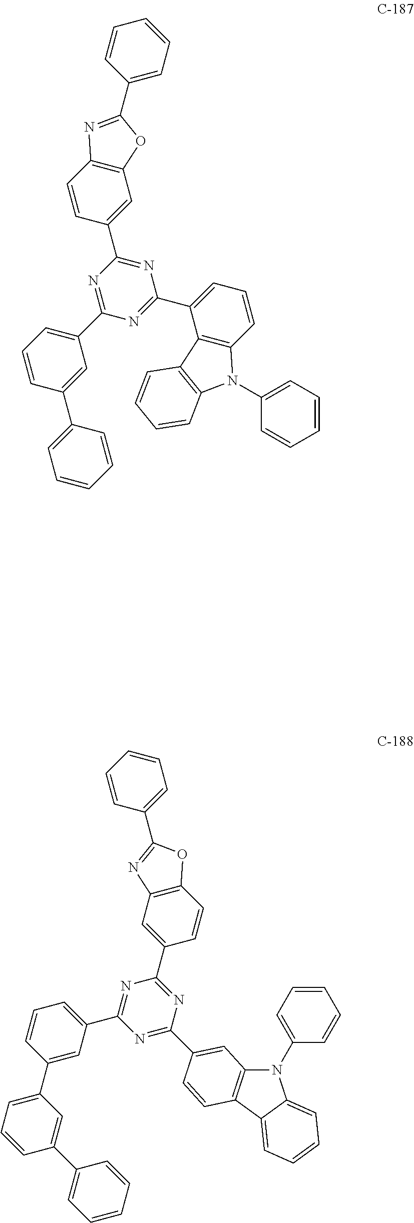 [SMILES: c1ccc(-c2cccc(-c3nc(-c4ccc5nc(-c6ccccc6)oc5c4)nc(-c4cccc5c4c4ccccc4n5-c4ccccc4)n3)c2)cc1 ; c1ccc(-c2cccc(-c3cccc(-c4nc(-c5ccc6oc(-c7ccccc7)nc6c5)nc(-c5ccc6c7ccccc7n(-c7ccccc7)c6c5)n4)c3)c2)cc1]

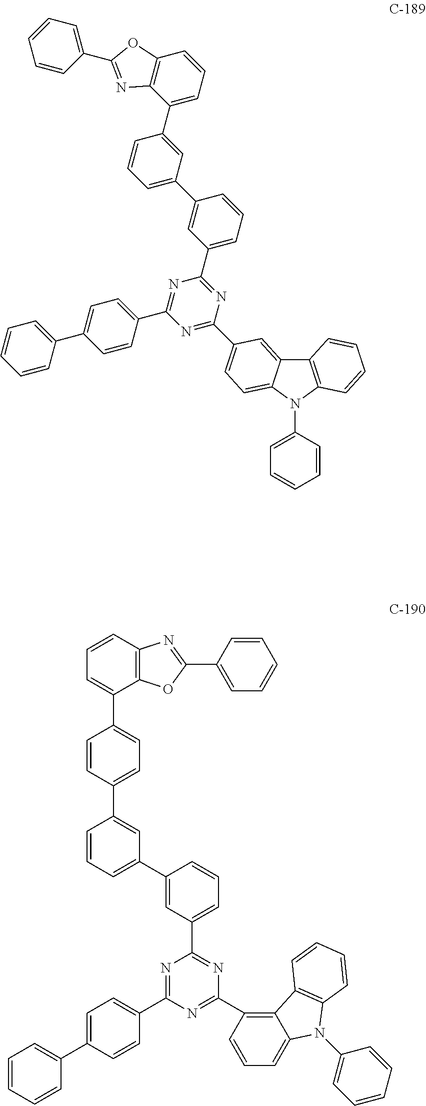 [SMILES: c1ccc(-c2ccc(-c3nc(-c4cccc(-c5cccc(-c6cccc7oc(-c8ccccc8)nc67)c5)c4)nc(-c4ccc5c(c4)c4ccccc4n5-c4ccccc4)n3)cc2)cc1 ; c1ccc(-c2ccc(-c3nc(-c4cccc(-c5cccc(-c6ccc(-c7cccc8nc(-c9ccccc9)oc78)cc6)c5)c4)nc(-c4cccc5c4c4ccccc4n5-c4ccccc4)n3)cc2)cc1]

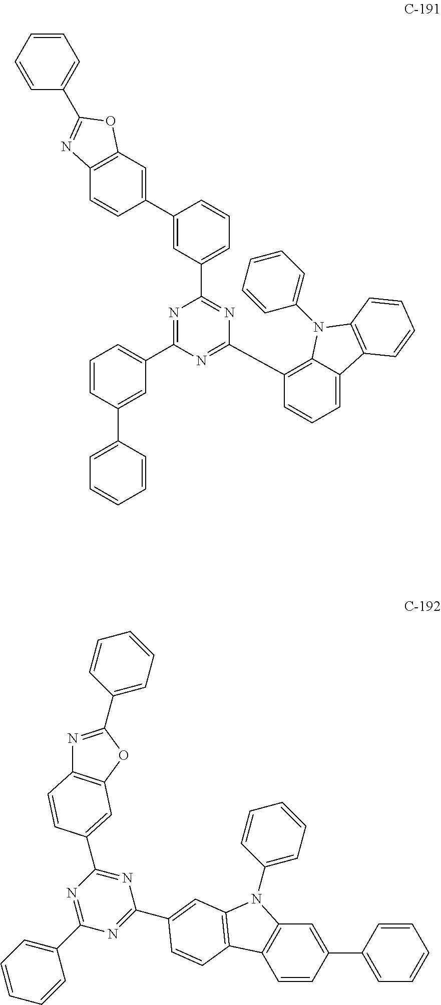 [SMILES: c1ccc(-c2cccc(-c3nc(-c4cccc(-c5ccc6nc(-c7ccccc7)oc6c5)c4)nc(-c4cccc5c6ccccc6n(-c6ccccc6)c45)n3)c2)cc1 ; c1ccc(-c2ccc3c4ccc(-c5nc(-c6ccccc6)nc(-c6ccc7nc(-c8ccccc8)oc7c6)n5)cc4n(-c4ccccc4)c3c2)cc1]

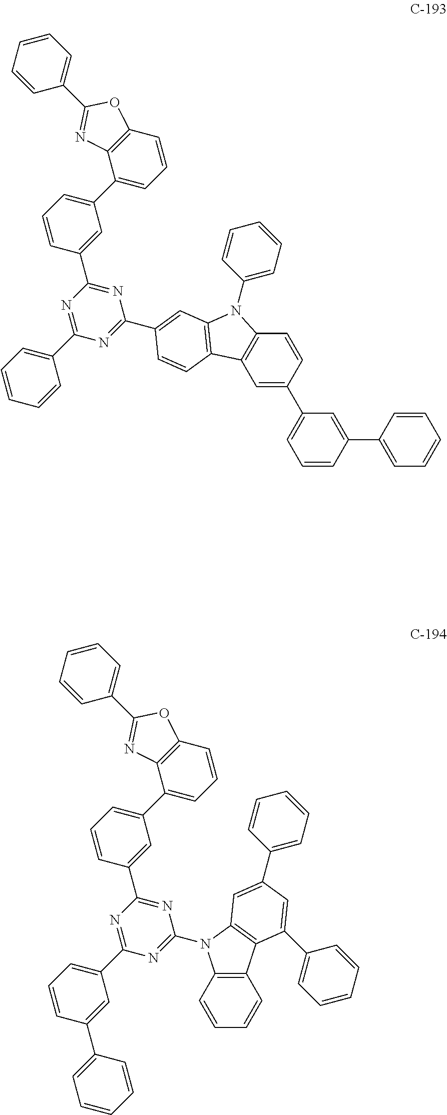 [SMILES: c1ccc(-c2cccc(-c3ccc4c(c3)c3ccc(-c5nc(-c6ccccc6)nc(-c6cccc(-c7cccc8oc(-c9ccccc9)nc78)c6)n5)cc3n4-c3ccccc3)c2)cc1 ; c1ccc(-c2cccc(-c3nc(-c4cccc(-c5cccc6oc(-c7ccccc7)nc56)c4)nc(-n4c5ccccc5c5c(-c6ccccc6)cc(-c6ccccc6)cc54)n3)c2)cc1]

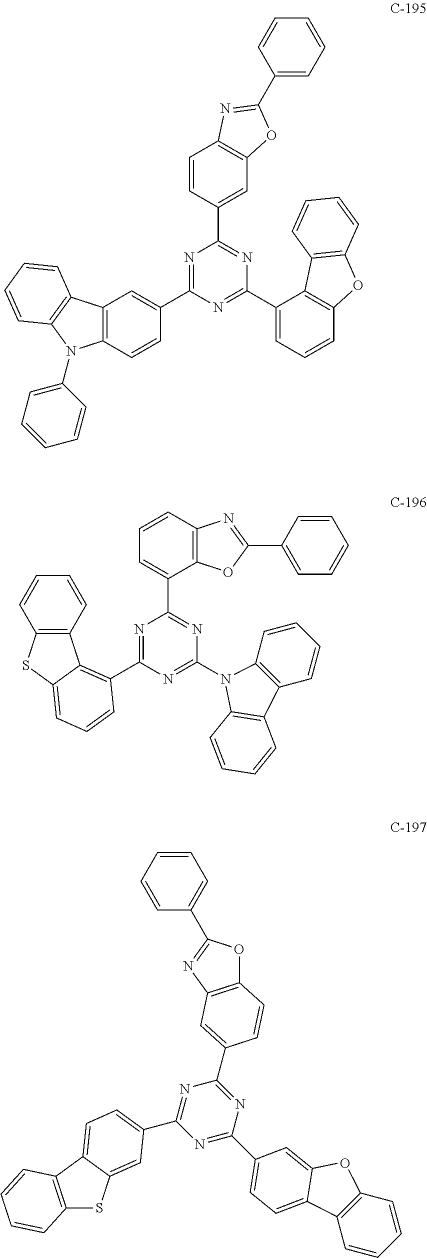 [SMILES: c1ccc(-c2nc3ccc(-c4nc(-c5ccc6c(c5)c5ccccc5n6-c5ccccc5)nc(-c5cccc6oc7ccccc7c56)n4)cc3o2)cc1 ; c1ccc(-c2nc3cccc(-c4nc(-c5cccc6sc7ccccc7c56)nc(-n5c6ccccc6c6ccccc65)n4)c3o2)cc1 ; c1ccc(-c2nc3cc(-c4nc(-c5ccc6c(c5)oc5ccccc56)nc(-c5ccc6c(c5)sc5ccccc56)n4)ccc3o2)cc1]

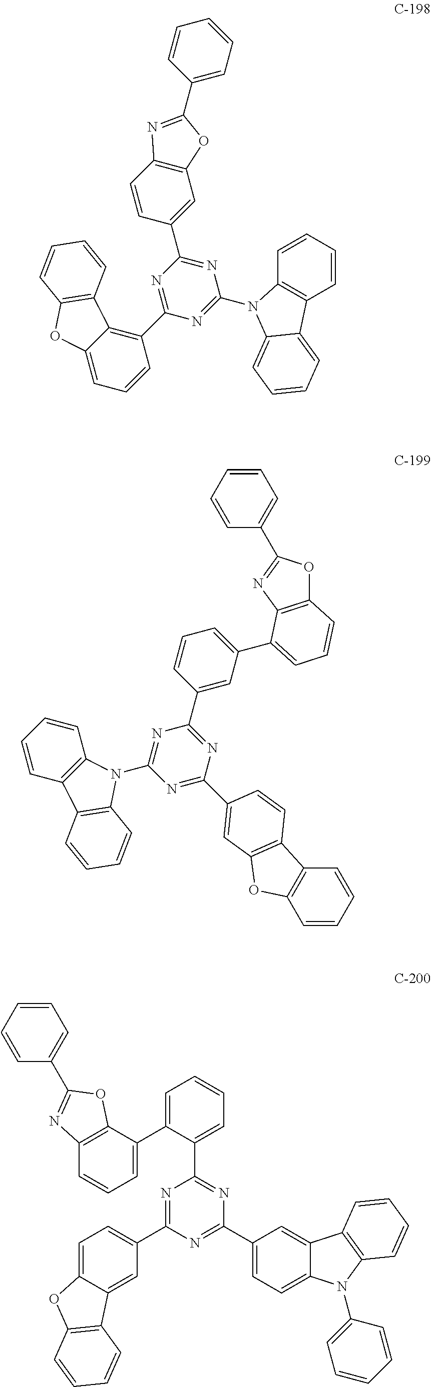 [SMILES: c1ccc(-c2nc3ccc(-c4nc(-c5cccc6oc7ccccc7c56)nc(-n5c6ccccc6c6ccccc65)n4)cc3o2)cc1 ; c1ccc(-c2nc3c(-c4cccc(-c5nc(-c6ccc7c(c6)oc6ccccc67)nc(-n6c7ccccc7c7ccccc76)n5)c4)cccc3o2)cc1 ; c1ccc(-c2nc3cccc(-c4ccccc4-c4nc(-c5ccc6oc7ccccc7c6c5)nc(-c5ccc6c(c5)c5ccccc5n6-c5ccccc5)n4)c3o2)cc1]

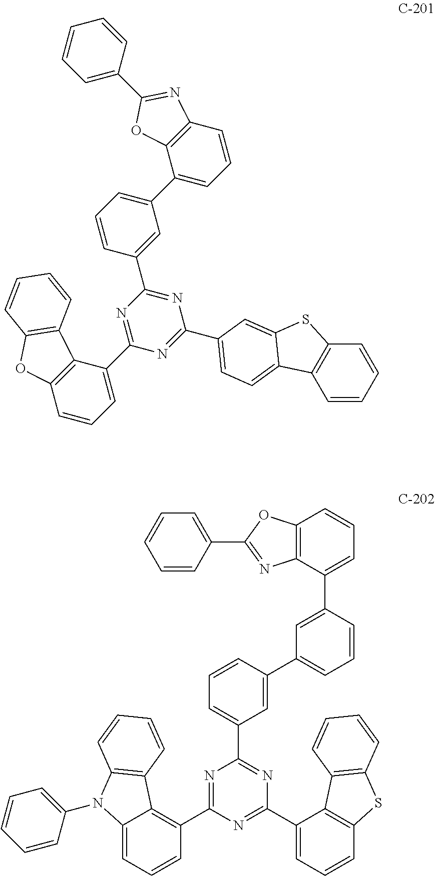 [SMILES: c1ccc(-c2nc3cccc(-c4cccc(-c5nc(-c6ccc7c(c6)sc6ccccc67)nc(-c6cccc7oc8ccccc8c67)n5)c4)c3o2)cc1 ; c1ccc(-c2nc3c(-c4cccc(-c5cccc(-c6nc(-c7cccc8sc9ccccc9c78)nc(-c7cccc8c7c7ccccc7n8-c7ccccc7)n6)c5)c4)cccc3o2)cc1]

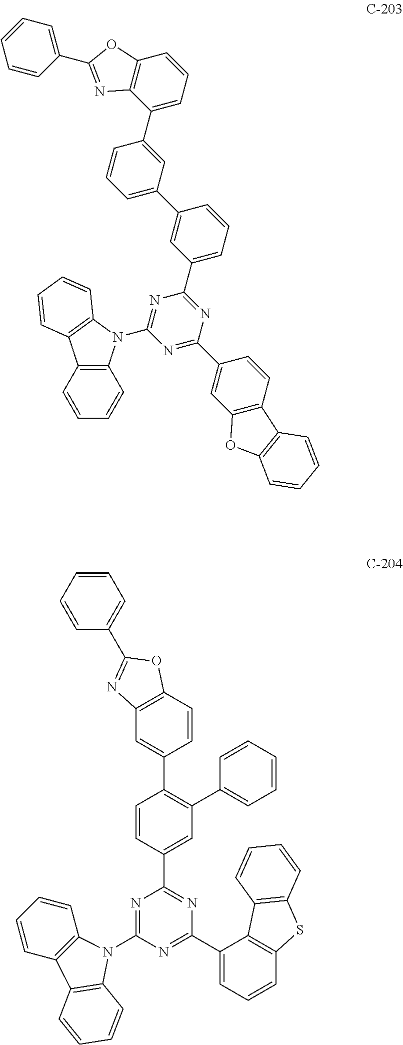 [SMILES: c1ccc(-c2nc3c(-c4cccc(-c5cccc(-c6nc(-c7ccc8c(c7)oc7ccccc78)nc(-n7c8ccccc8c8ccccc87)n6)c5)c4)cccc3o2)cc1 ; c1ccc(-c2nc3cc(-c4ccc(-c5nc(-c6cccc7sc8ccccc8c67)nc(-n6c7ccccc7c7ccccc76)n5)cc4-c4ccccc4)ccc3o2)cc1]

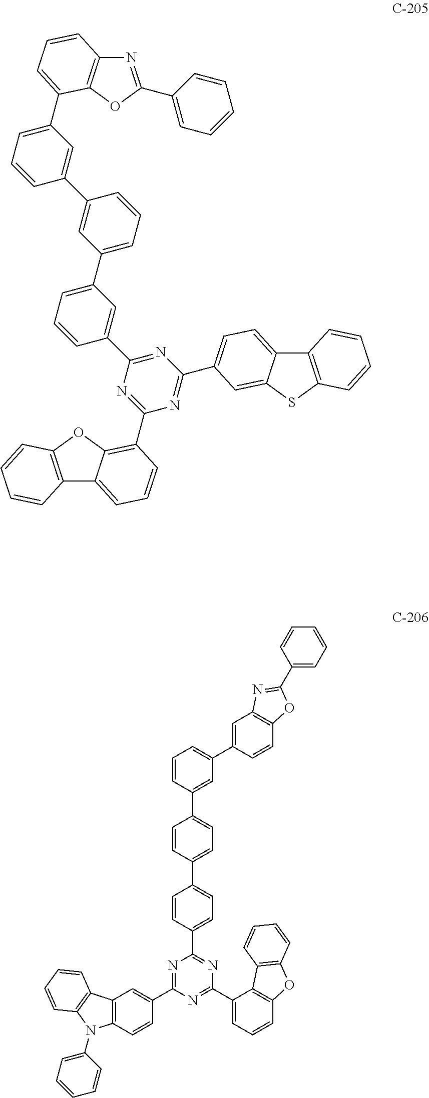 [SMILES: c1ccc(-c2nc3cccc(-c4cccc(-c5cccc(-c6cccc(-c7nc(-c8ccc9c(c8)sc8ccccc89)nc(-c8cccc9c8oc8ccccc89)n7)c6)c5)c4)c3o2)cc1 ; c1ccc(-c2nc3cc(-c4cccc(-c5ccc(-c6ccc(-c7nc(-c8ccc9c(c8)c8ccccc8n9-c8ccccc8)nc(-c8cccc9oc%10ccccc%10c89)n7)cc6)cc5)c4)ccc3o2)cc1]

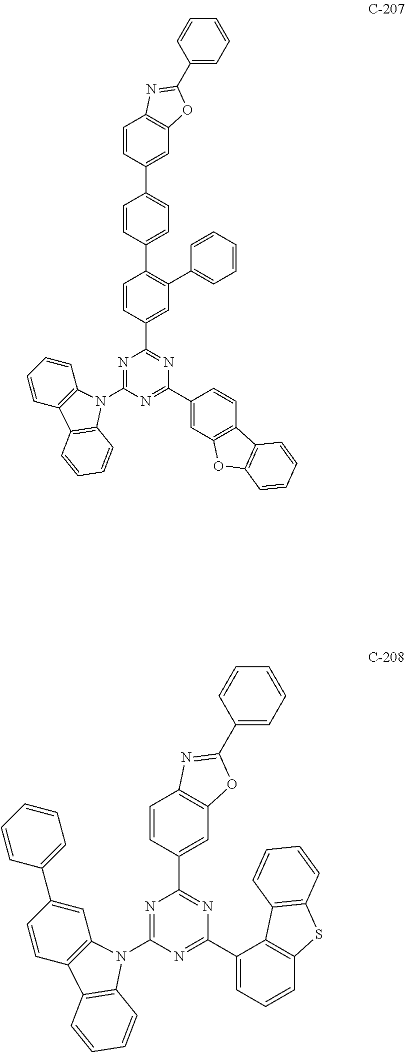 [SMILES: c1ccc(-c2nc3ccc(-c4ccc(-c5ccc(-c6nc(-c7ccc8c(c7)oc7ccccc78)nc(-n7c8ccccc8c8ccccc87)n6)cc5-c5ccccc5)cc4)cc3o2)cc1 ; c1ccc(-c2ccc3c4ccccc4n(-c4nc(-c5ccc6nc(-c7ccccc7)oc6c5)nc(-c5cccc6sc7ccccc7c56)n4)c3c2)cc1]

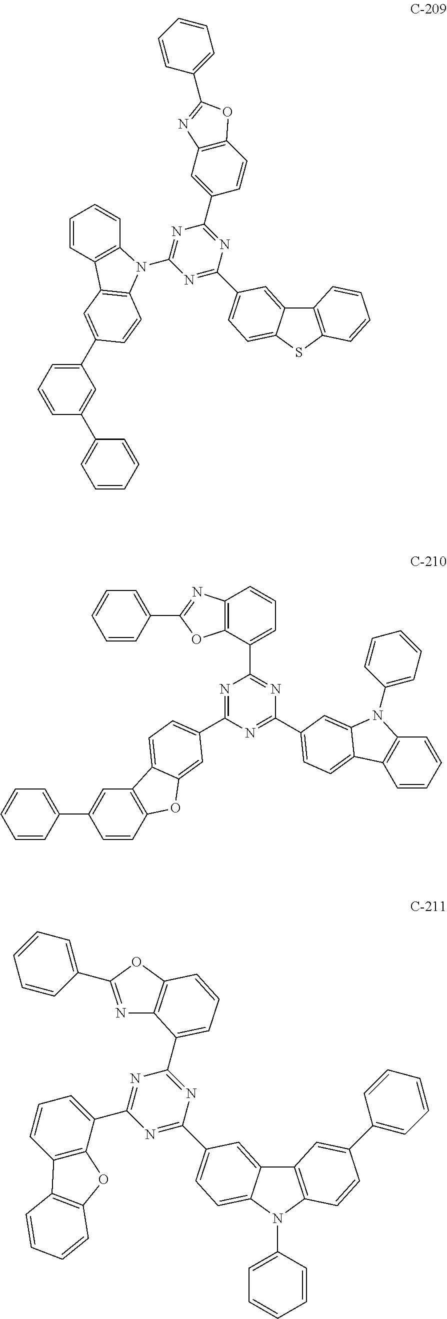 [SMILES: c1ccc(-c2cccc(-c3ccc4c(c3)c3ccccc3n4-c3nc(-c4ccc5oc(-c6ccccc6)nc5c4)nc(-c4ccc5sc6ccccc6c5c4)n3)c2)cc1 ; c1ccc(-c2ccc3oc4cc(-c5nc(-c6ccc7c8ccccc8n(-c8ccccc8)c7c6)nc(-c6cccc7nc(-c8ccccc8)oc67)n5)ccc4c3c2)cc1 ; c1ccc(-c2ccc3c(c2)c2cc(-c4nc(-c5cccc6oc(-c7ccccc7)nc56)nc(-c5cccc6c5oc5ccccc56)n4)ccc2n3-c2ccccc2)cc1]

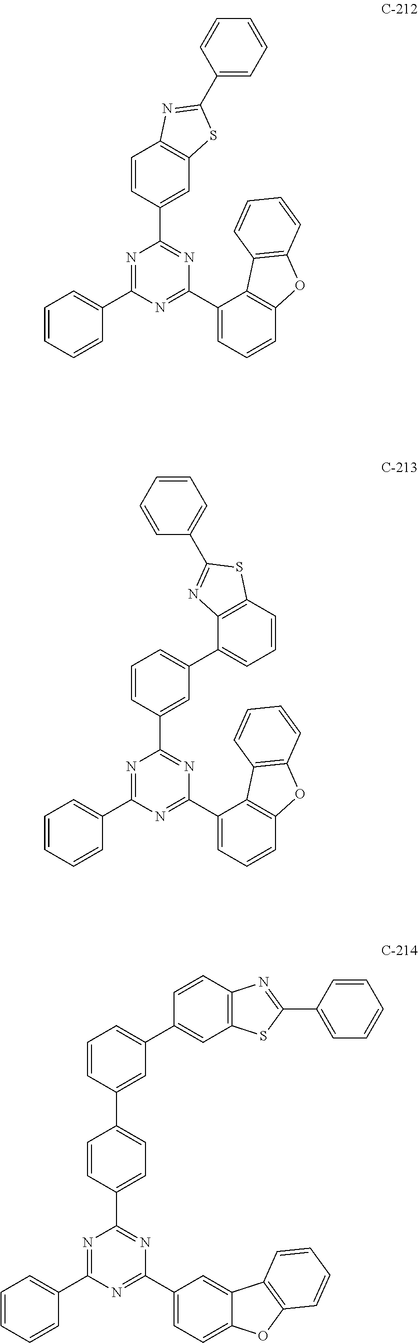 [SMILES: c1ccc(-c2nc(-c3ccc4nc(-c5ccccc5)sc4c3)nc(-c3cccc4oc5ccccc5c34)n2)cc1 ; c1ccc(-c2nc(-c3cccc(-c4cccc5sc(-c6ccccc6)nc45)c3)nc(-c3cccc4oc5ccccc5c34)n2)cc1 ; c1ccc(-c2nc(-c3ccc(-c4cccc(-c5ccc6nc(-c7ccccc7)sc6c5)c4)cc3)nc(-c3ccc4oc5ccccc5c4c3)n2)cc1]

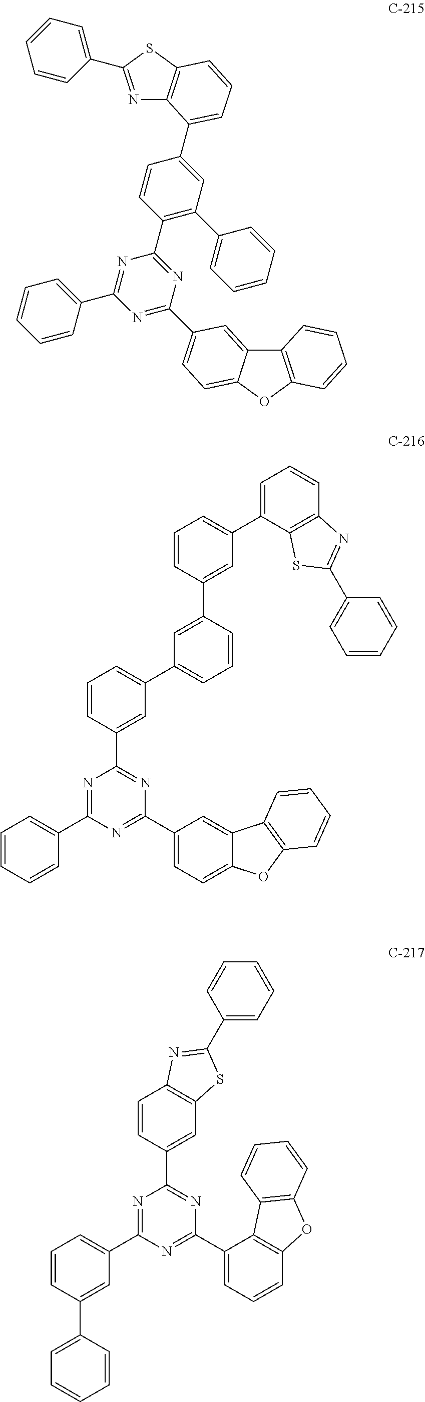 [SMILES: c1ccc(-c2nc(-c3ccc4oc5ccccc5c4c3)nc(-c3ccc(-c4cccc5sc(-c6ccccc6)nc45)cc3-c3ccccc3)n2)cc1 ; c1ccc(-c2nc(-c3cccc(-c4cccc(-c5cccc(-c6cccc7nc(-c8ccccc8)sc67)c5)c4)c3)nc(-c3ccc4oc5ccccc5c4c3)n2)cc1 ; c1ccc(-c2cccc(-c3nc(-c4ccc5nc(-c6ccccc6)sc5c4)nc(-c4cccc5oc6ccccc6c45)n3)c2)cc1]

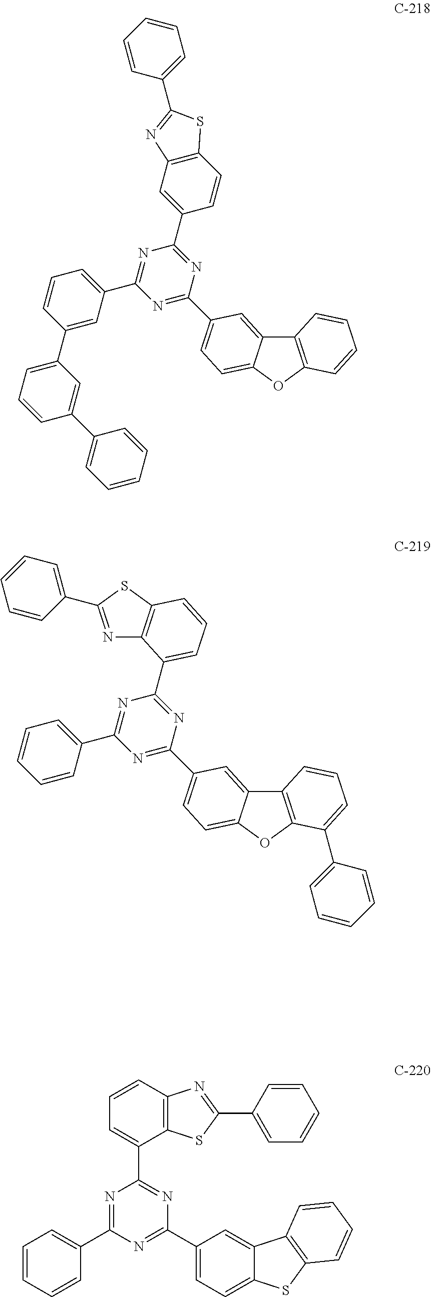 [SMILES: c1ccc(-c2cccc(-c3cccc(-c4nc(-c5ccc6sc(-c7ccccc7)nc6c5)nc(-c5ccc6oc7ccccc7c6c5)n4)c3)c2)cc1 ; c1ccc(-c2nc(-c3ccc4oc5c(-c6ccccc6)cccc5c4c3)nc(-c3cccc4sc(-c5ccccc5)nc34)n2)cc1 ; c1ccc(-c2nc(-c3ccc4sc5ccccc5c4c3)nc(-c3cccc4nc(-c5ccccc5)sc34)n2)cc1]

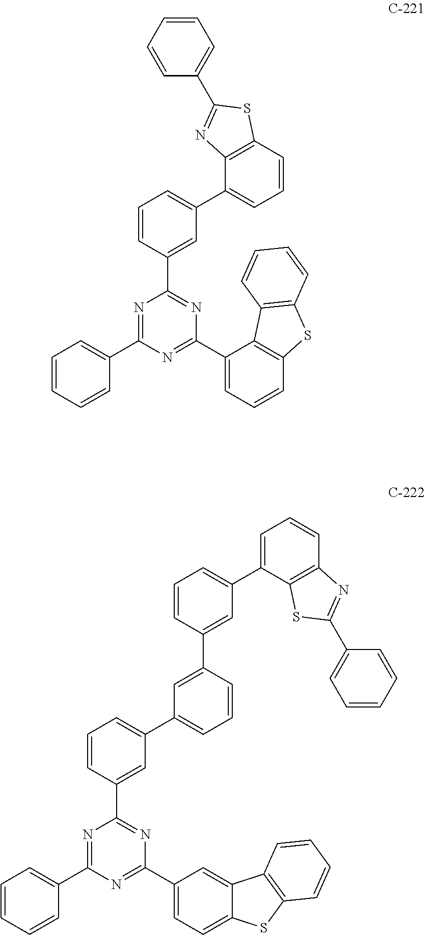 [SMILES: c1ccc(-c2nc(-c3cccc(-c4cccc5sc(-c6ccccc6)nc45)c3)nc(-c3cccc4sc5ccccc5c34)n2)cc1 ; c1ccc(-c2nc(-c3cccc(-c4cccc(-c5cccc(-c6cccc7nc(-c8ccccc8)sc67)c5)c4)c3)nc(-c3ccc4sc5ccccc5c4c3)n2)cc1]

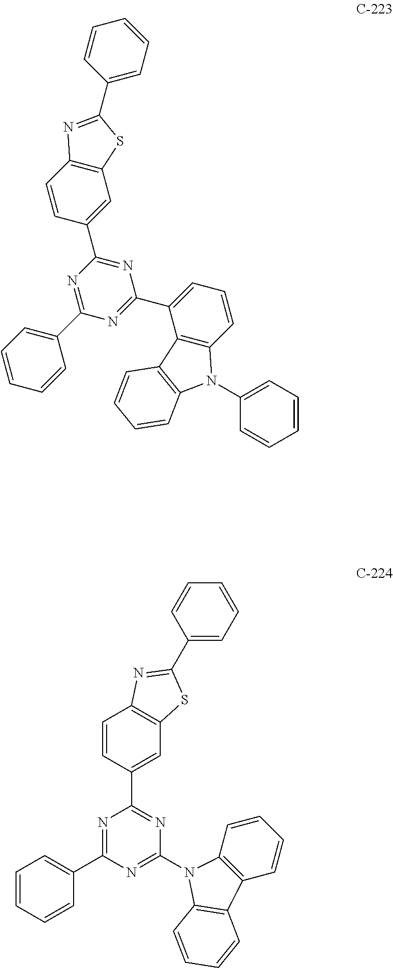 [SMILES: c1ccc(-c2nc(-c3ccc4nc(-c5ccccc5)sc4c3)nc(-c3cccc4c3c3ccccc3n4-c3ccccc3)n2)cc1 ; c1ccc(-c2nc(-c3ccc4nc(-c5ccccc5)sc4c3)nc(-n3c4ccccc4c4ccccc43)n2)cc1]

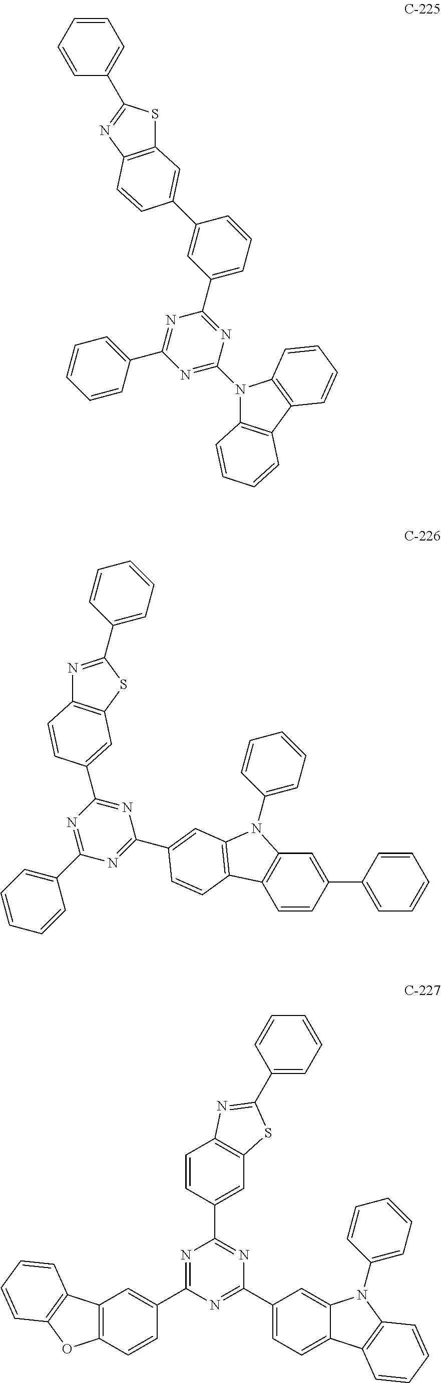 [SMILES: c1ccc(-c2nc(-c3cccc(-c4ccc5nc(-c6ccccc6)sc5c4)c3)nc(-n3c4ccccc4c4ccccc43)n2)cc1 ; c1ccc(-c2ccc3c4ccc(-c5nc(-c6ccccc6)nc(-c6ccc7nc(-c8ccccc8)sc7c6)n5)cc4n(-c4ccccc4)c3c2)cc1 ; c1ccc(-c2nc3ccc(-c4nc(-c5ccc6oc7ccccc7c6c5)nc(-c5ccc6c7ccccc7n(-c7ccccc7)c6c5)n4)cc3s2)cc1]

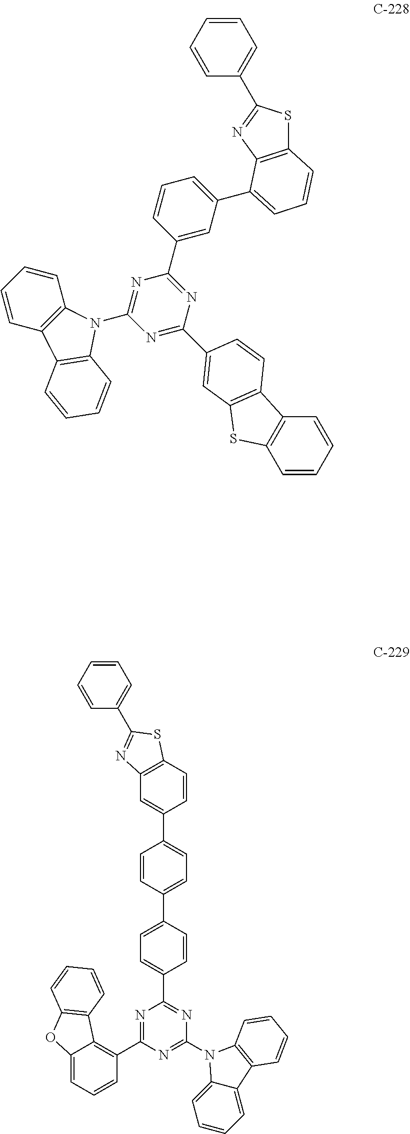 [SMILES: c1ccc(-c2nc3c(-c4cccc(-c5nc(-c6ccc7c(c6)sc6ccccc67)nc(-n6c7ccccc7c7ccccc76)n5)c4)cccc3s2)cc1 ; c1ccc(-c2nc3cc(-c4ccc(-c5ccc(-c6nc(-c7cccc8oc9ccccc9c78)nc(-n7c8ccccc8c8ccccc87)n6)cc5)cc4)ccc3s2)cc1]

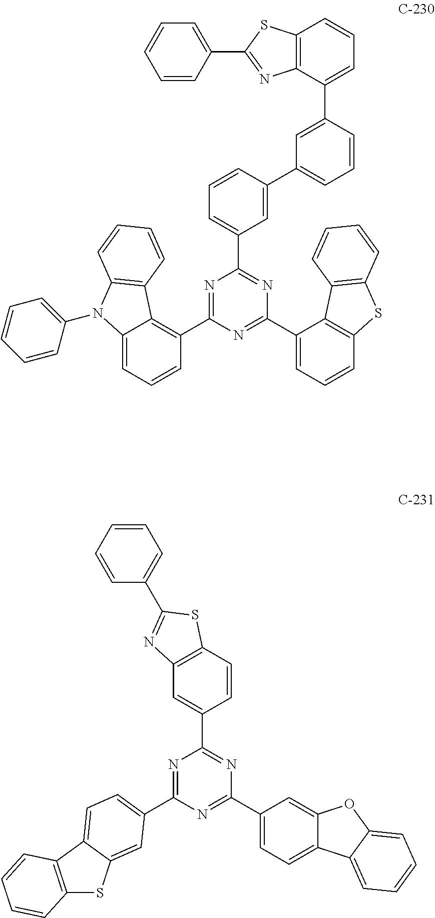 [SMILES: c1ccc(-c2nc3c(-c4cccc(-c5cccc(-c6nc(-c7cccc8sc9ccccc9c78)nc(-c7cccc8c7c7ccccc7n8-c7ccccc7)n6)c5)c4)cccc3s2)cc1 ; c1ccc(-c2nc3cc(-c4nc(-c5ccc6c(c5)oc5ccccc56)nc(-c5ccc6c(c5)sc5ccccc56)n4)ccc3s2)cc1]

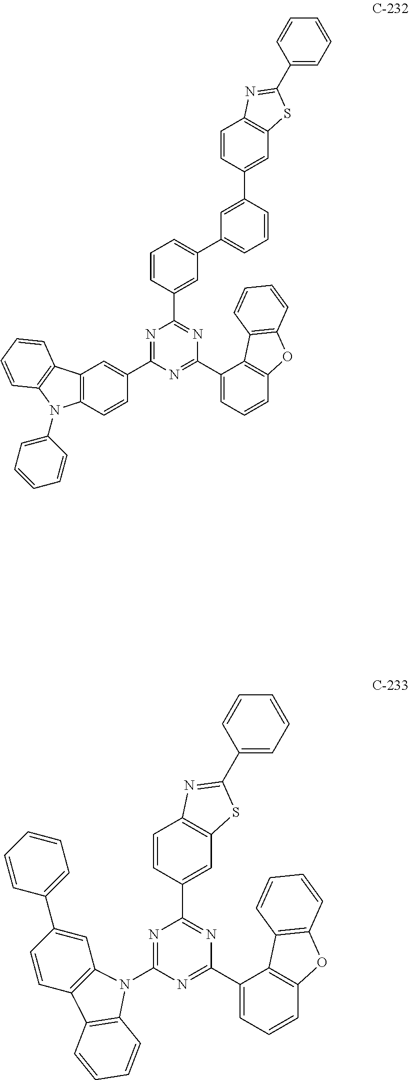 [SMILES: c1ccc(-c2nc3ccc(-c4cccc(-c5cccc(-c6nc(-c7ccc8c(c7)c7ccccc7n8-c7ccccc7)nc(-c7cccc8oc9ccccc9c78)n6)c5)c4)cc3s2)cc1 ; c1ccc(-c2ccc3c4ccccc4n(-c4nc(-c5ccc6nc(-c7ccccc7)sc6c5)nc(-c5cccc6oc7ccccc7c56)n4)c3c2)cc1]

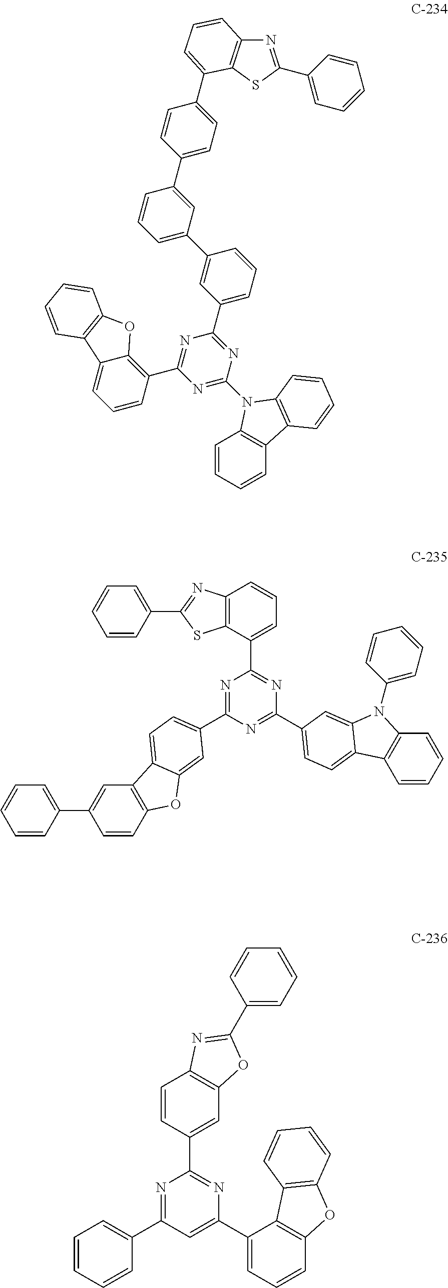 [SMILES: c1ccc(-c2nc3cccc(-c4ccc(-c5cccc(-c6cccc(-c7nc(-c8cccc9c8oc8ccccc89)nc(-n8c9ccccc9c9ccccc98)n7)c6)c5)cc4)c3s2)cc1 ; c1ccc(-c2ccc3oc4cc(-c5nc(-c6ccc7c8ccccc8n(-c8ccccc8)c7c6)nc(-c6cccc7nc(-c8ccccc8)sc67)n5)ccc4c3c2)cc1 ; c1ccc(-c2cc(-c3cccc4oc5ccccc5c34)nc(-c3ccc4nc(-c5ccccc5)oc4c3)n2)cc1]

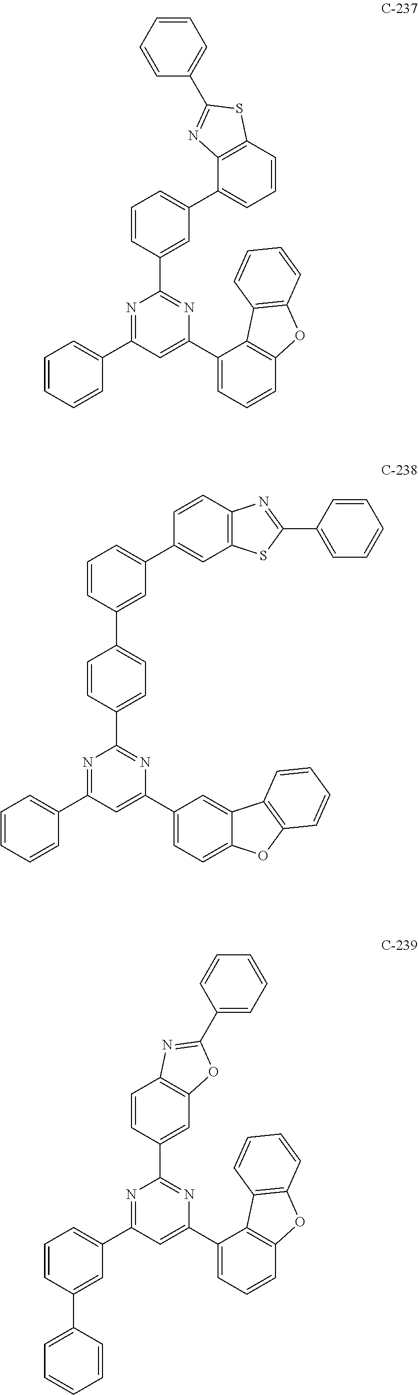 [SMILES: c1ccc(-c2cc(-c3cccc4oc5ccccc5c34)nc(-c3cccc(-c4cccc5sc(-c6ccccc6)nc45)c3)n2)cc1 ; c1ccc(-c2cc(-c3ccc4oc5ccccc5c4c3)nc(-c3ccc(-c4cccc(-c5ccc6nc(-c7ccccc7)sc6c5)c4)cc3)n2)cc1 ; c1ccc(-c2cccc(-c3cc(-c4cccc5oc6ccccc6c45)nc(-c4ccc5nc(-c6ccccc6)oc5c4)n3)c2)cc1]

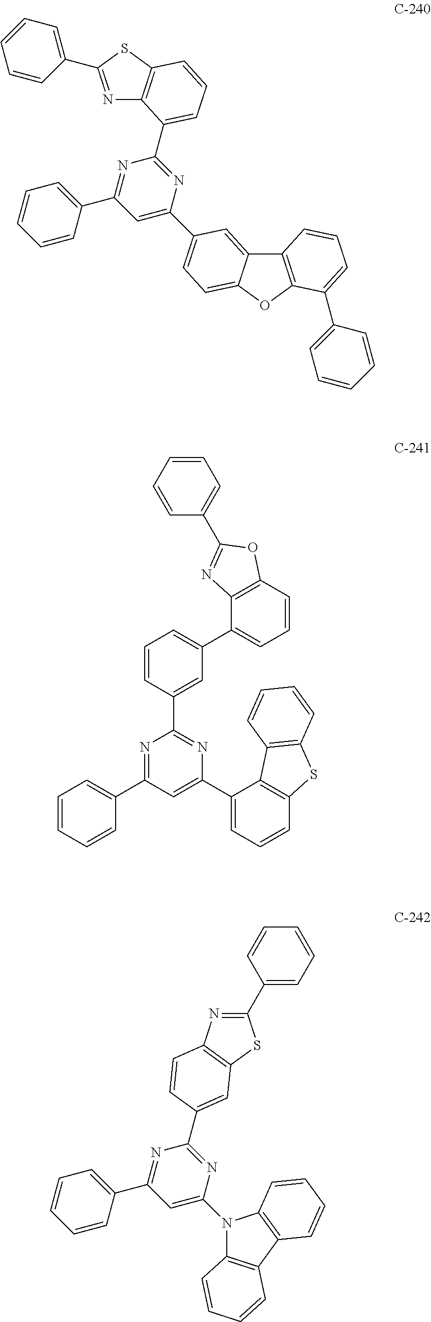 [SMILES: c1ccc(-c2cc(-c3ccc4oc5c(-c6ccccc6)cccc5c4c3)nc(-c3cccc4sc(-c5ccccc5)nc34)n2)cc1 ; c1ccc(-c2cc(-c3cccc4sc5ccccc5c34)nc(-c3cccc(-c4cccc5oc(-c6ccccc6)nc45)c3)n2)cc1 ; c1ccc(-c2cc(-n3c4ccccc4c4ccccc43)nc(-c3ccc4nc(-c5ccccc5)sc4c3)n2)cc1]

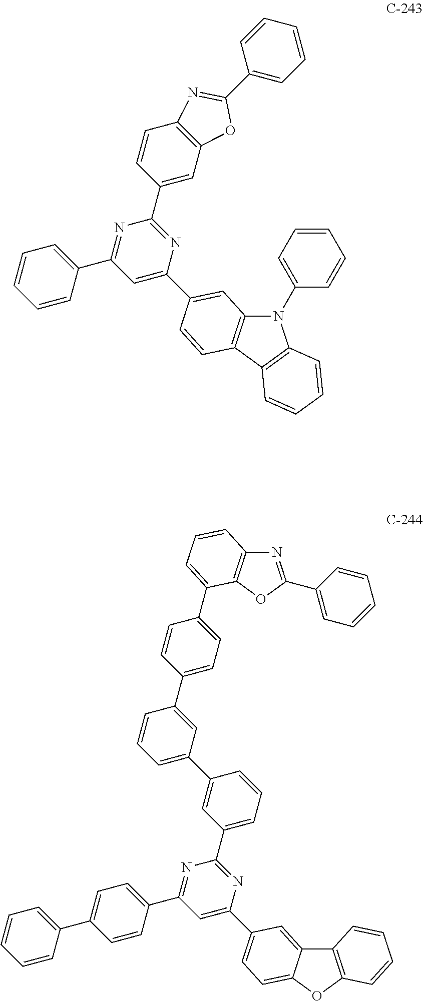 [SMILES: c1ccc(-c2cc(-c3ccc4c5ccccc5n(-c5ccccc5)c4c3)nc(-c3ccc4nc(-c5ccccc5)oc4c3)n2)cc1 ; c1ccc(-c2ccc(-c3cc(-c4ccc5oc6ccccc6c5c4)nc(-c4cccc(-c5cccc(-c6ccc(-c7cccc8nc(-c9ccccc9)oc78)cc6)c5)c4)n3)cc2)cc1]

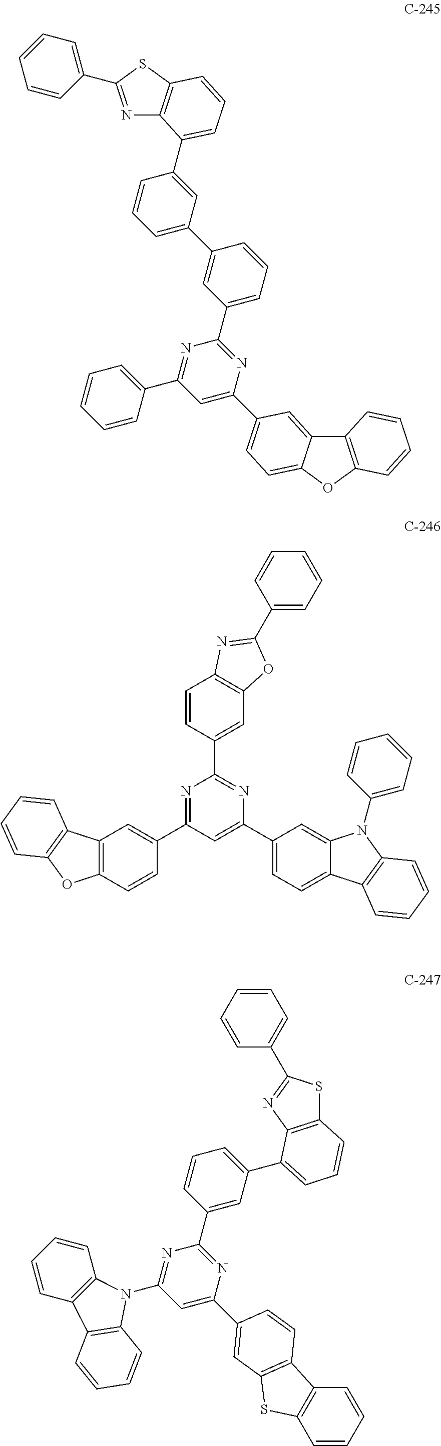 [SMILES: c1ccc(-c2cc(-c3ccc4oc5ccccc5c4c3)nc(-c3cccc(-c4cccc(-c5cccc6sc(-c7ccccc7)nc56)c4)c3)n2)cc1 ; c1ccc(-c2nc3ccc(-c4nc(-c5ccc6oc7ccccc7c6c5)cc(-c5ccc6c7ccccc7n(-c7ccccc7)c6c5)n4)cc3o2)cc1 ; c1ccc(-c2nc3c(-c4cccc(-c5nc(-c6ccc7c(c6)sc6ccccc67)cc(-n6c7ccccc7c7ccccc76)n5)c4)cccc3s2)cc1]

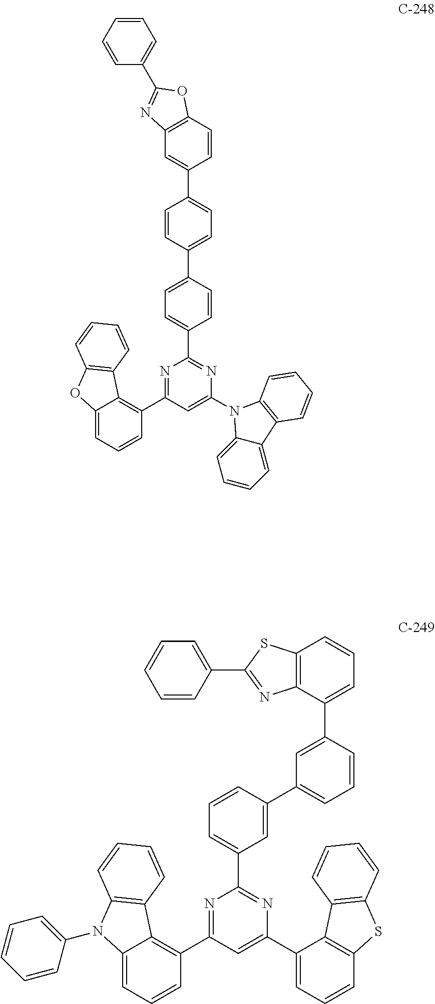 [SMILES: c1ccc(-c2nc3cc(-c4ccc(-c5ccc(-c6nc(-c7cccc8oc9ccccc9c78)cc(-n7c8ccccc8c8ccccc87)n6)cc5)cc4)ccc3o2)cc1 ; c1ccc(-c2nc3c(-c4cccc(-c5cccc(-c6nc(-c7cccc8sc9ccccc9c78)cc(-c7cccc8c7c7ccccc7n8-c7ccccc7)n6)c5)c4)cccc3s2)cc1]

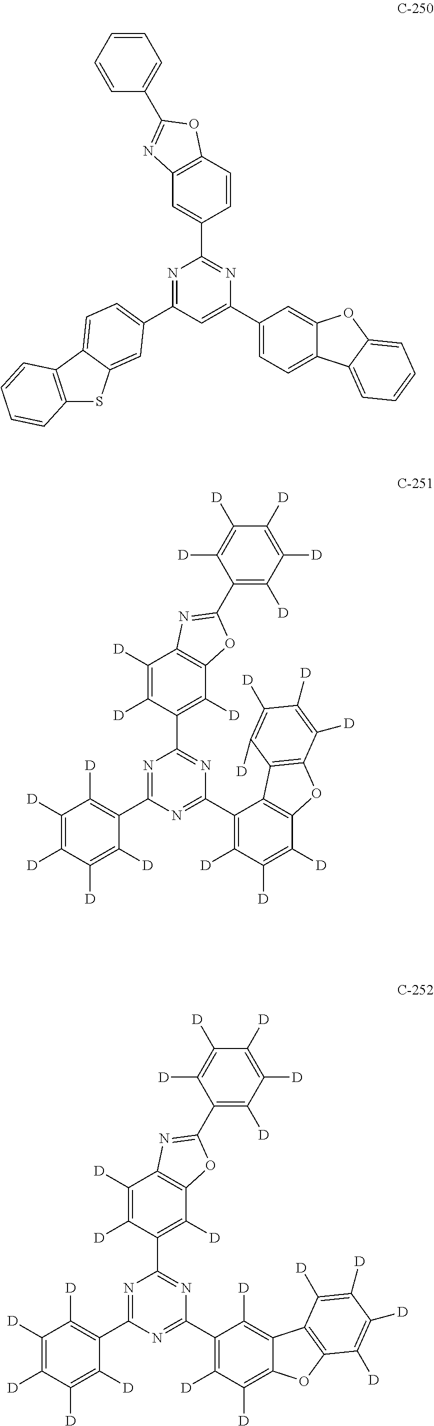 [SMILES: c1ccc(-c2nc3cc(-c4nc(-c5ccc6c(c5)oc5ccccc56)cc(-c5ccc6c(c5)sc5ccccc56)n4)ccc3o2)cc1 ; [2H]c1c([2H])c([2H])c(-c2nc(-c3c([2H])c([2H])c4nc(-c5c([2H])c([2H])c([2H])c([2H])c5[2H])oc4c3[2H])nc(-c3c([2H])c([2H])c([2H])c4oc5c([2H])c([2H])c([2H])c([2H])c5c34)n2)c([2H])c1[2H] ; [2H]c1c([2H])c([2H])c(-c2nc(-c3c([2H])c([2H])c4nc(-c5c([2H])c([2H])c([2H])c([2H])c5[2H])oc4c3[2H])nc(-c3c([2H])c([2H])c4oc5c([2H])c([2H])c([2H])c([2H])c5c4c3[2H])n2)c([2H])c1[2H]]

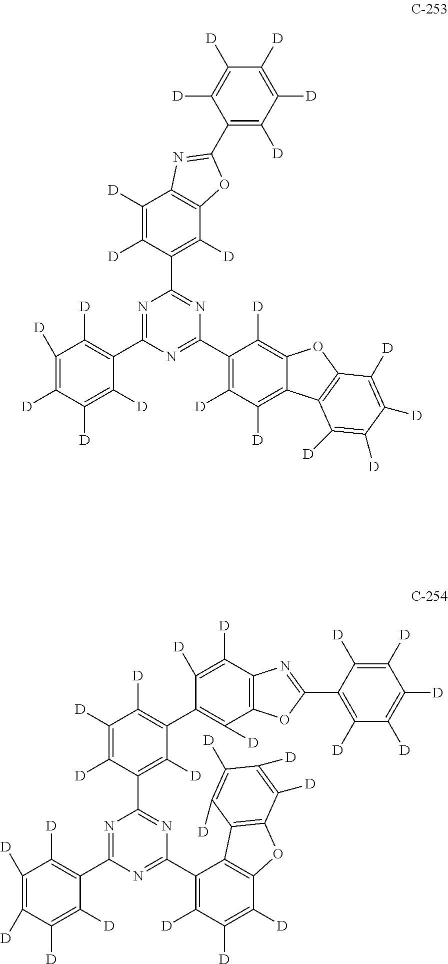 [SMILES: [2H]c1c([2H])c([2H])c(-c2nc(-c3c([2H])c([2H])c4nc(-c5c([2H])c([2H])c([2H])c([2H])c5[2H])oc4c3[2H])nc(-c3c([2H])c([2H])c4c(oc5c([2H])c([2H])c([2H])c([2H])c54)c3[2H])n2)c([2H])c1[2H] ; [2H]c1c([2H])c([2H])c(-c2nc(-c3c([2H])c([2H])c([2H])c(-c4c([2H])c([2H])c5nc(-c6c([2H])c([2H])c([2H])c([2H])c6[2H])oc5c4[2H])c3[2H])nc(-c3c([2H])c([2H])c([2H])c4oc5c([2H])c([2H])c([2H])c([2H])c5c34)n2)c([2H])c1[2H]]

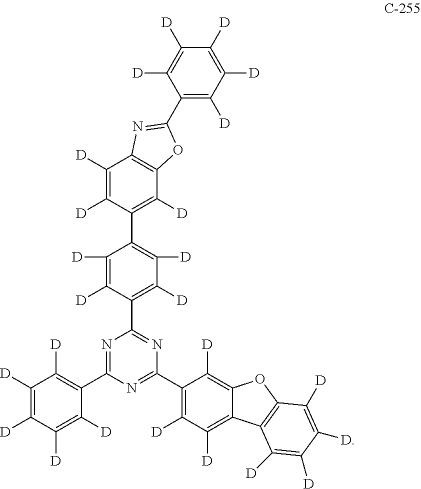

10. The plurality of host materials according to claim 1, wherein the compound represented by formula 2 is at least one selected from the following compounds: 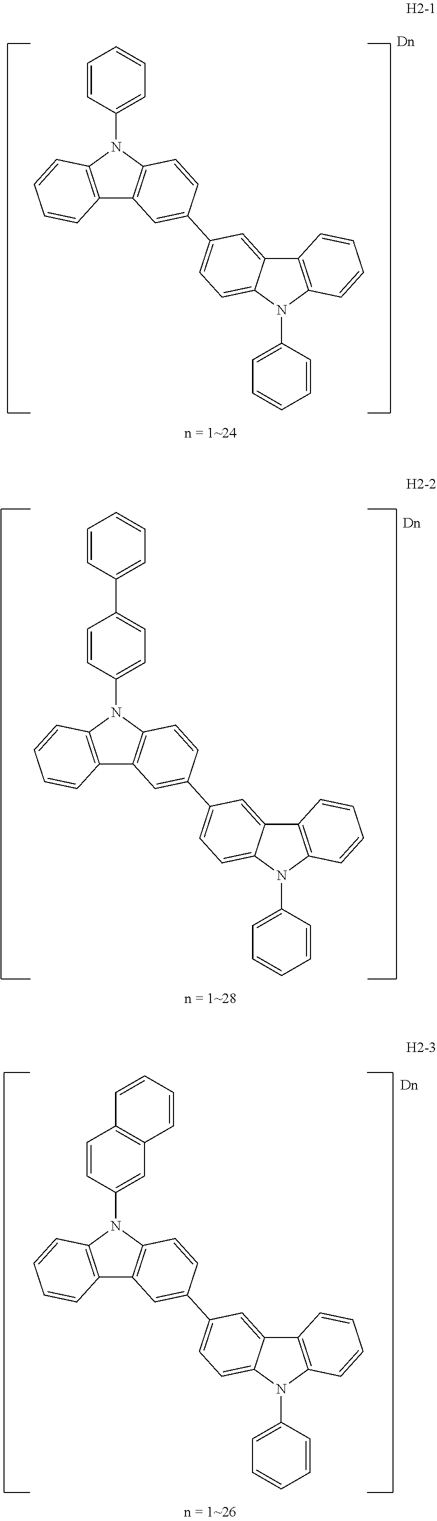 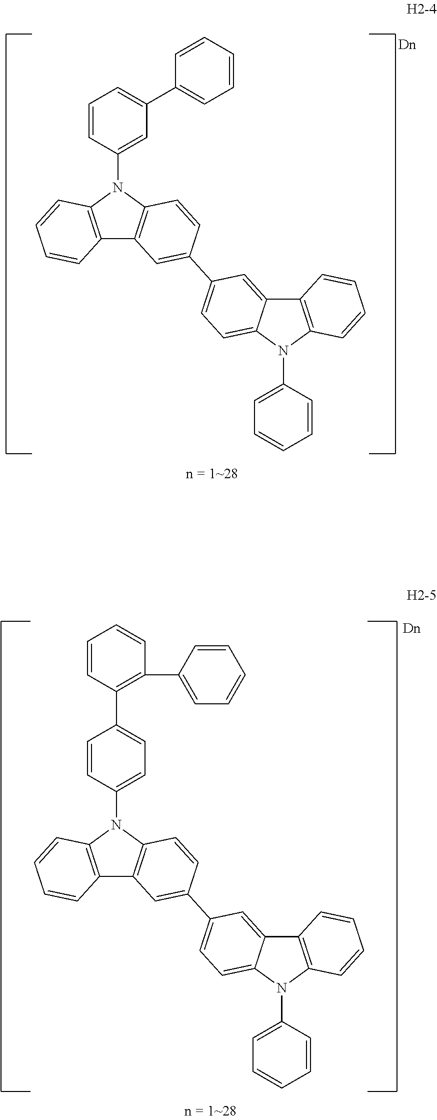 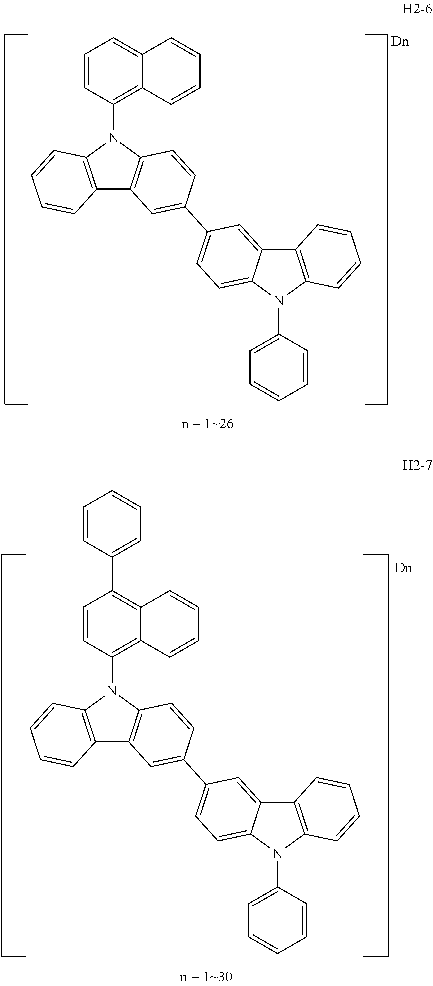 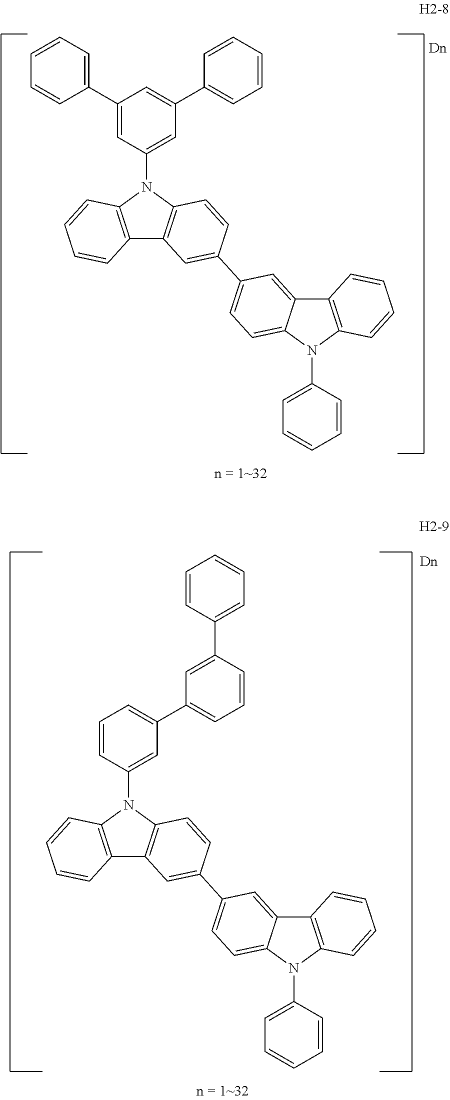 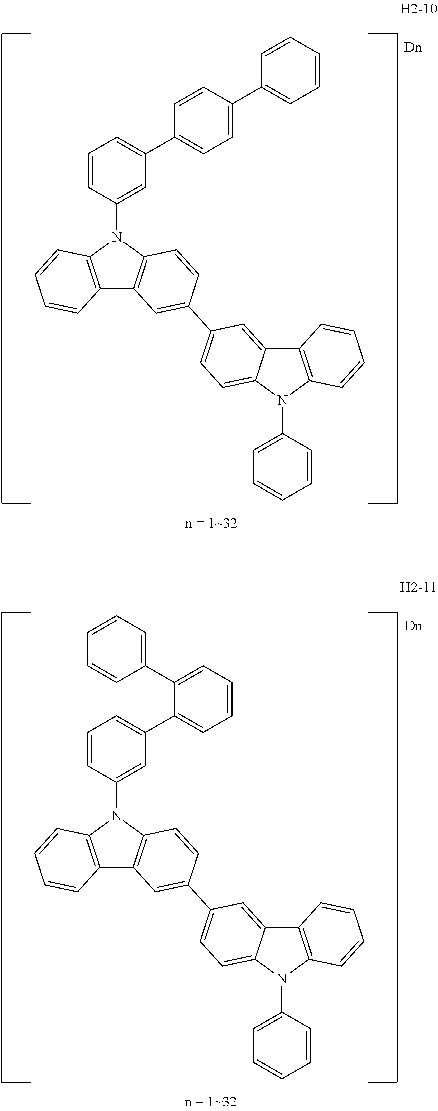 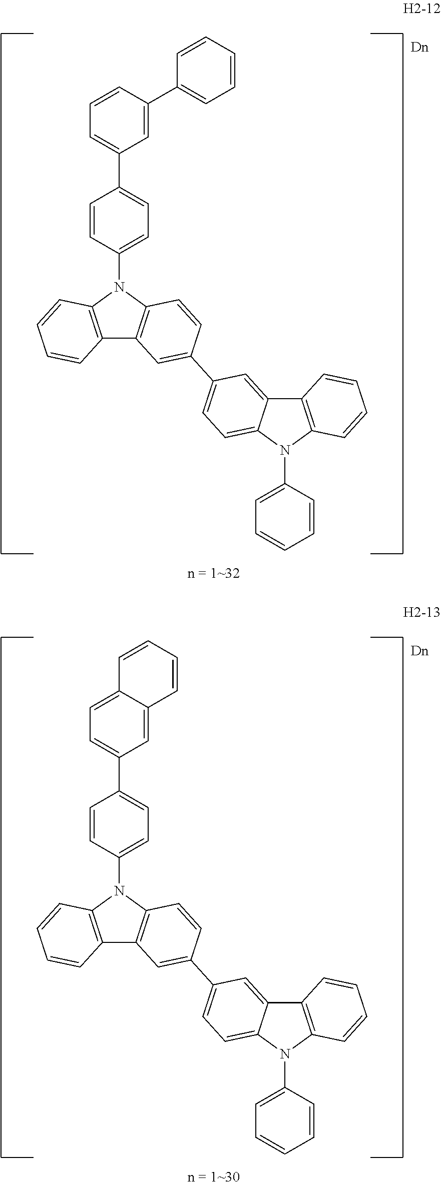 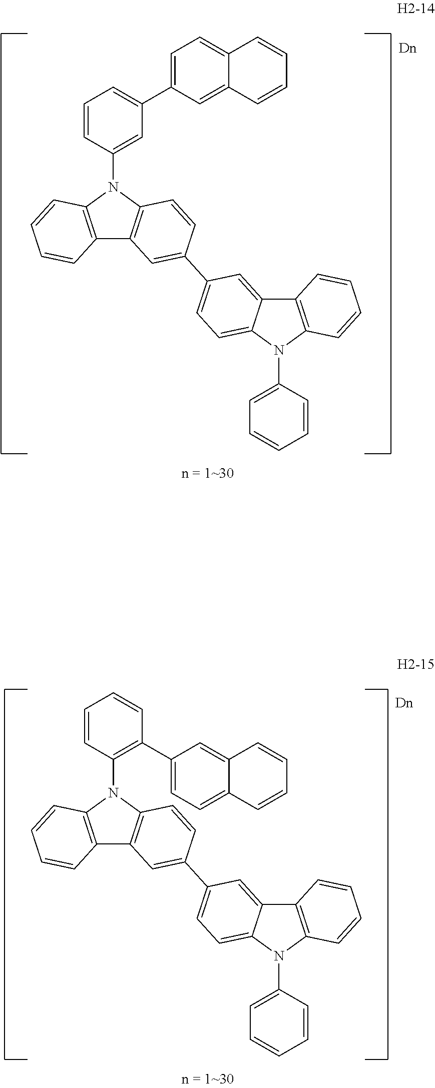 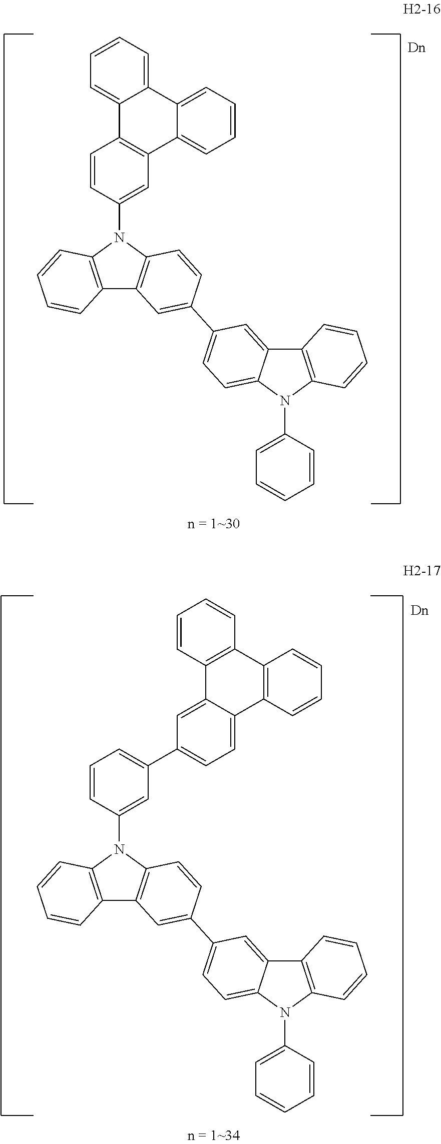 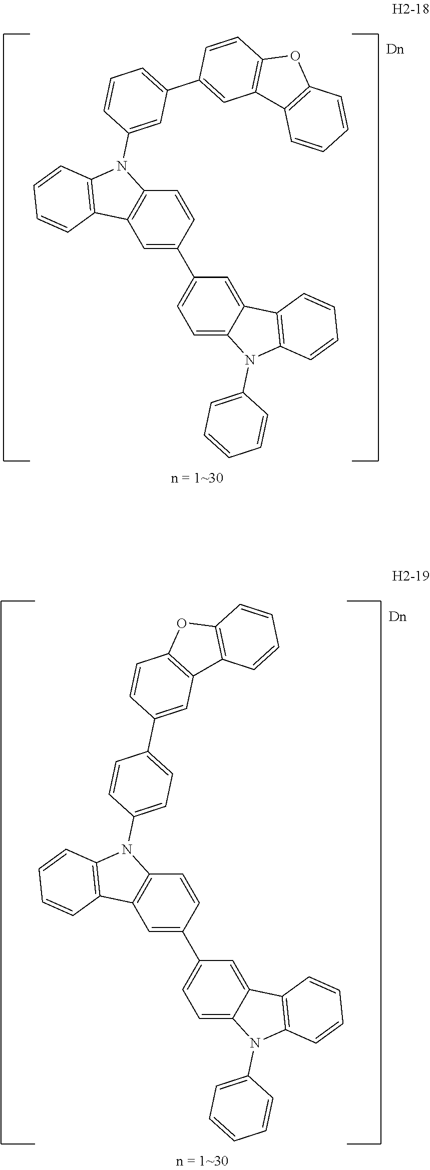 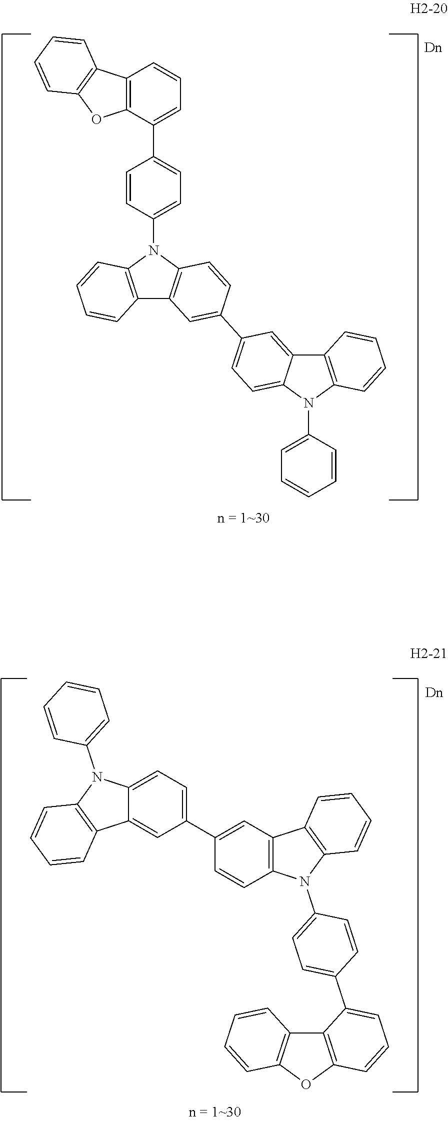 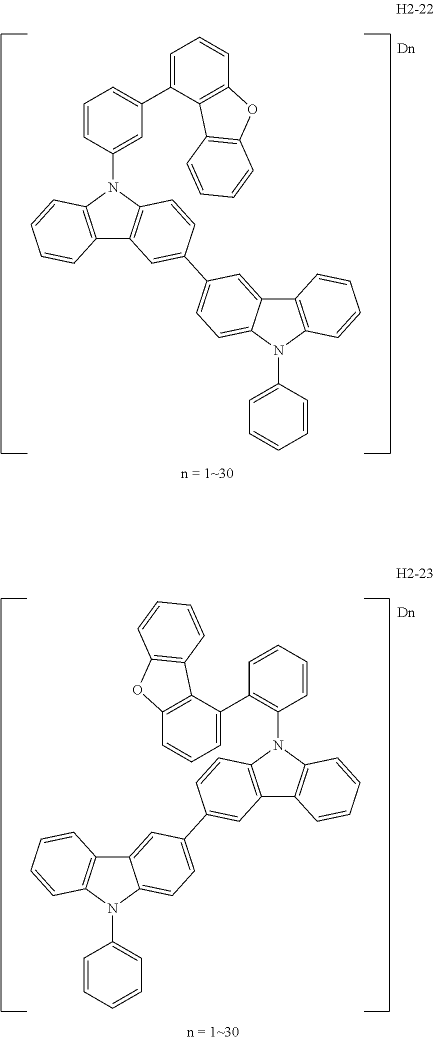 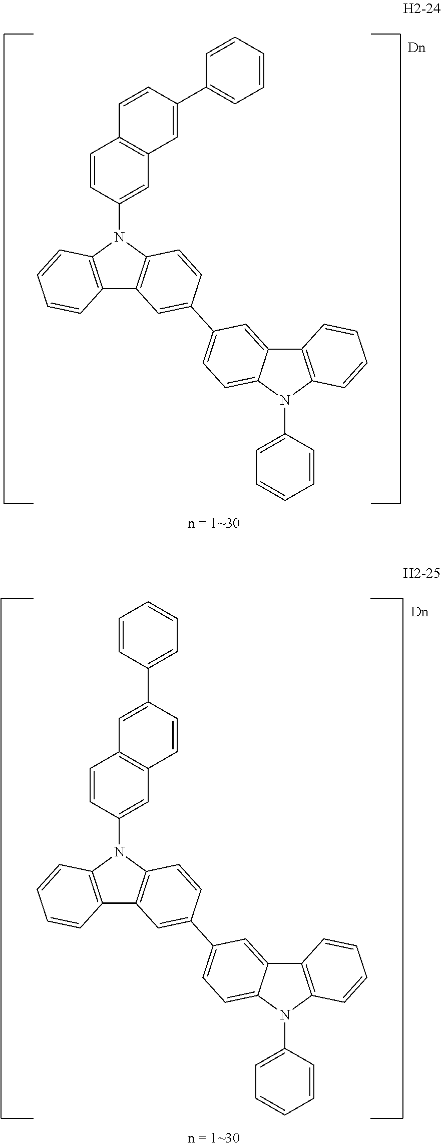 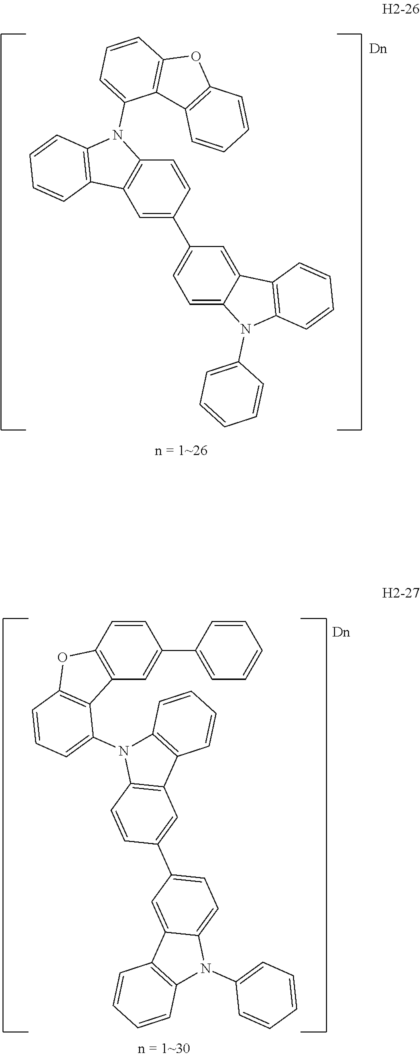 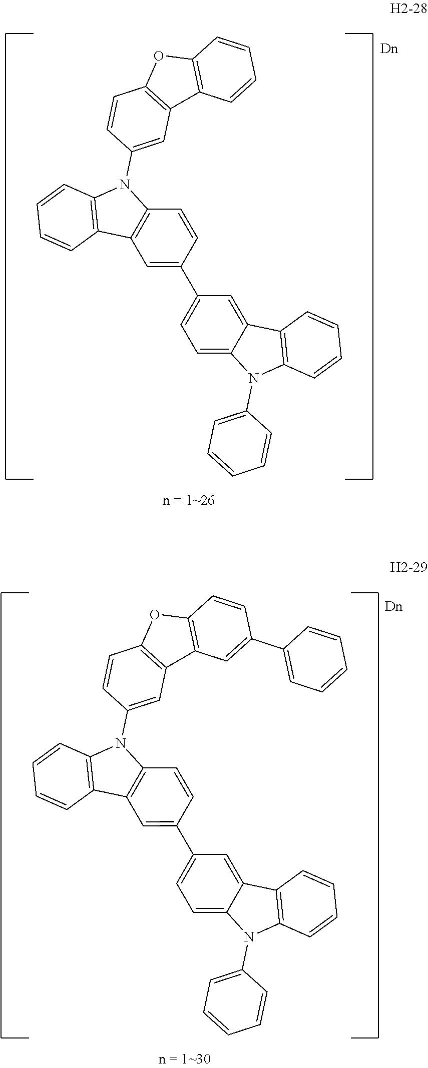 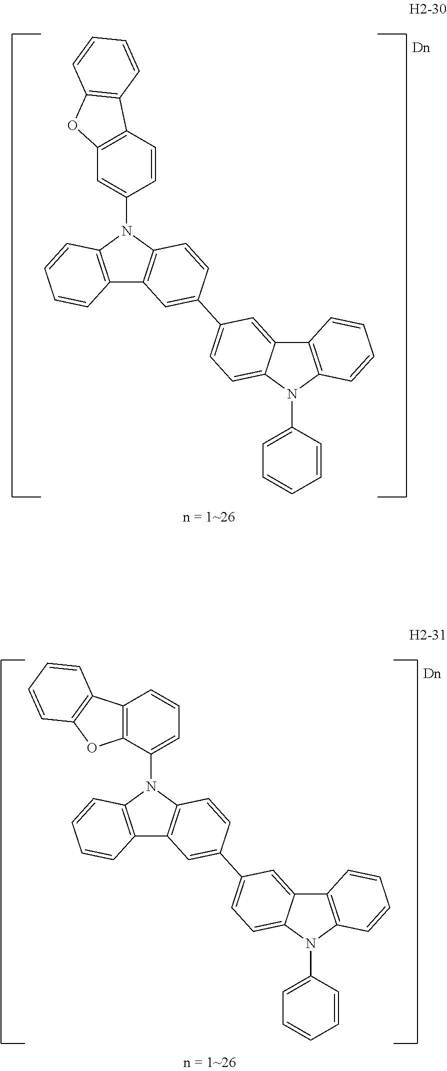 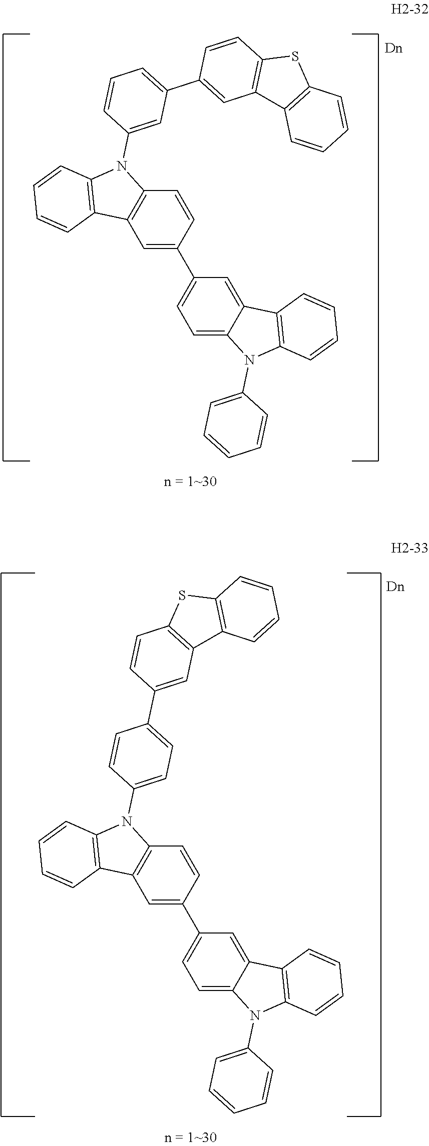 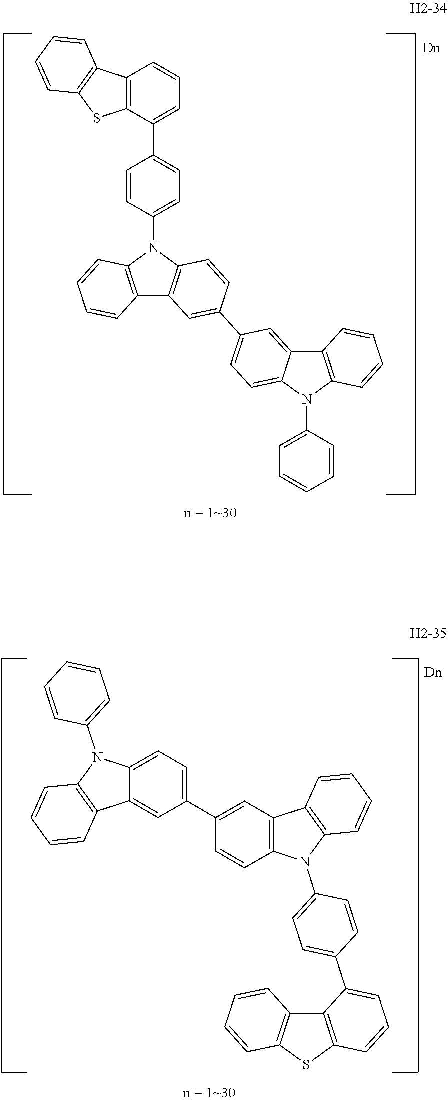 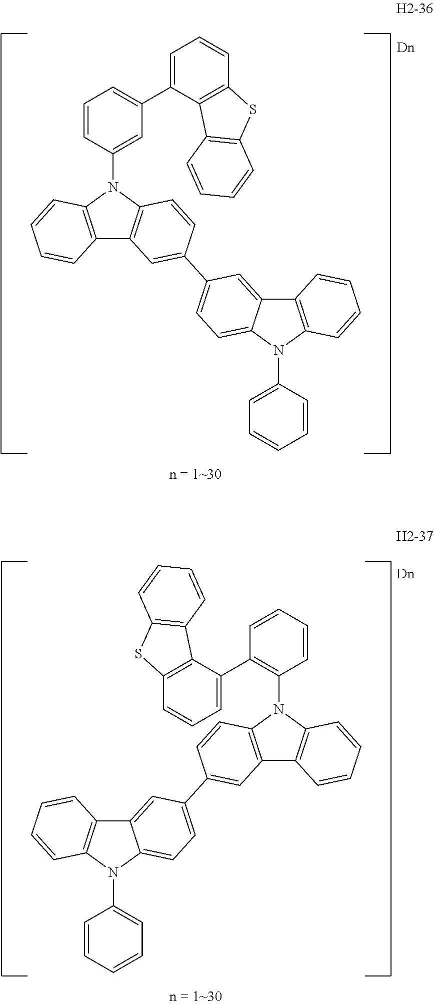 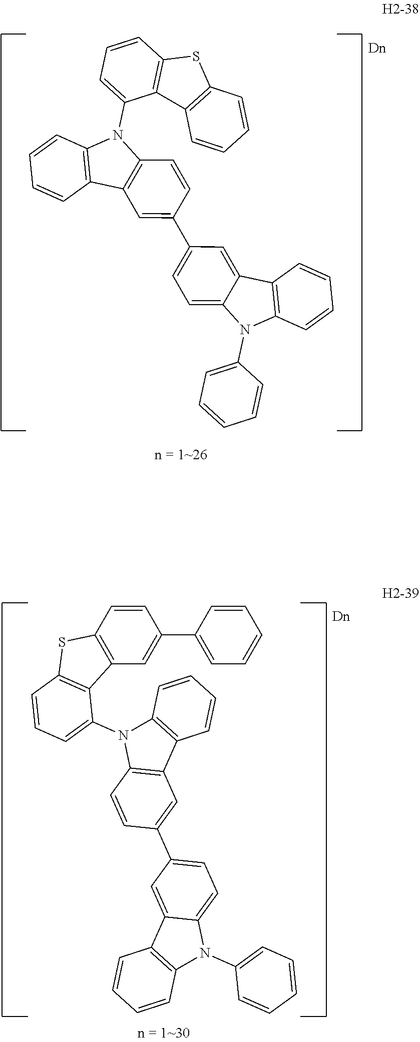 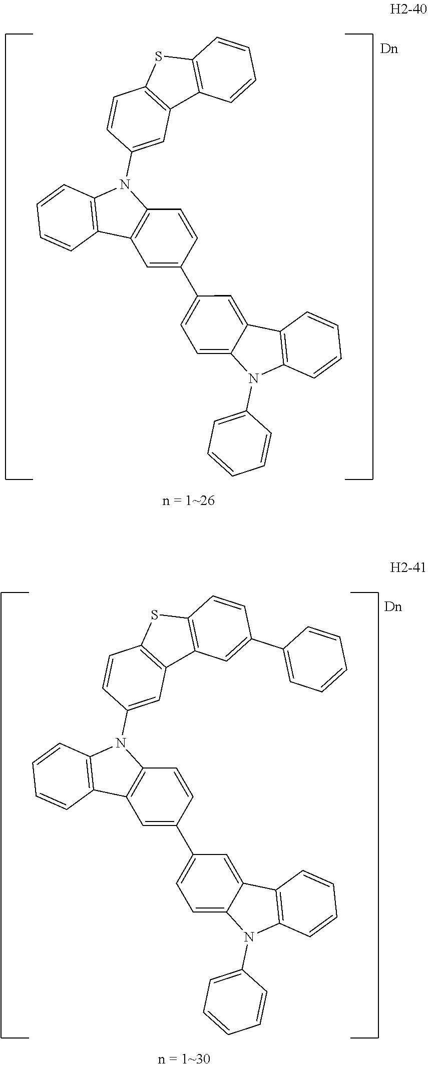 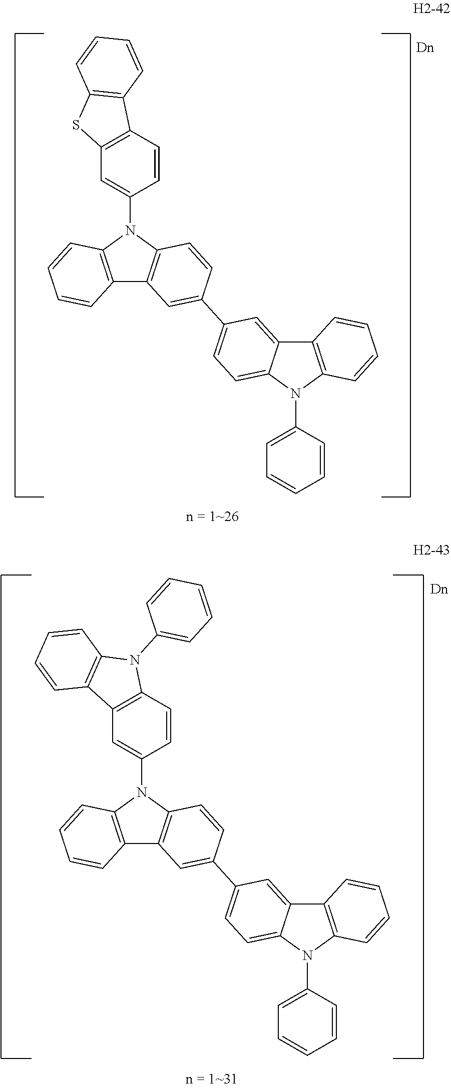 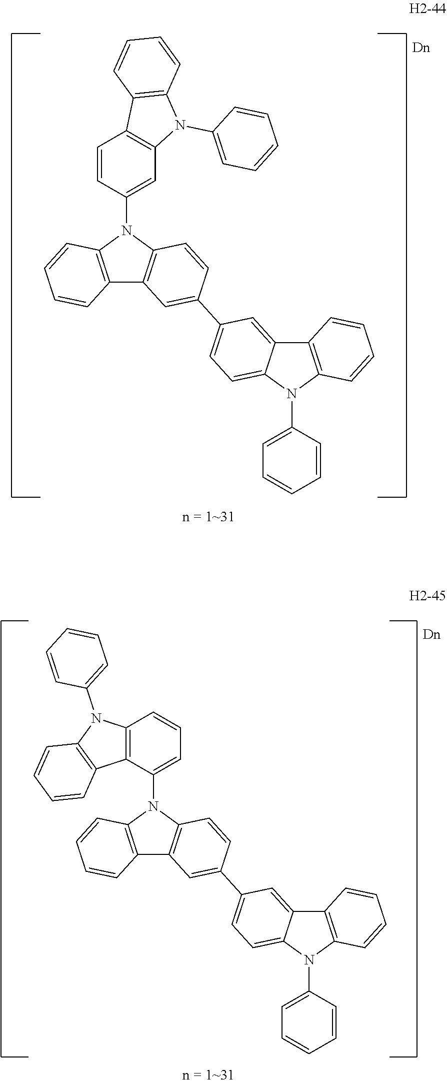 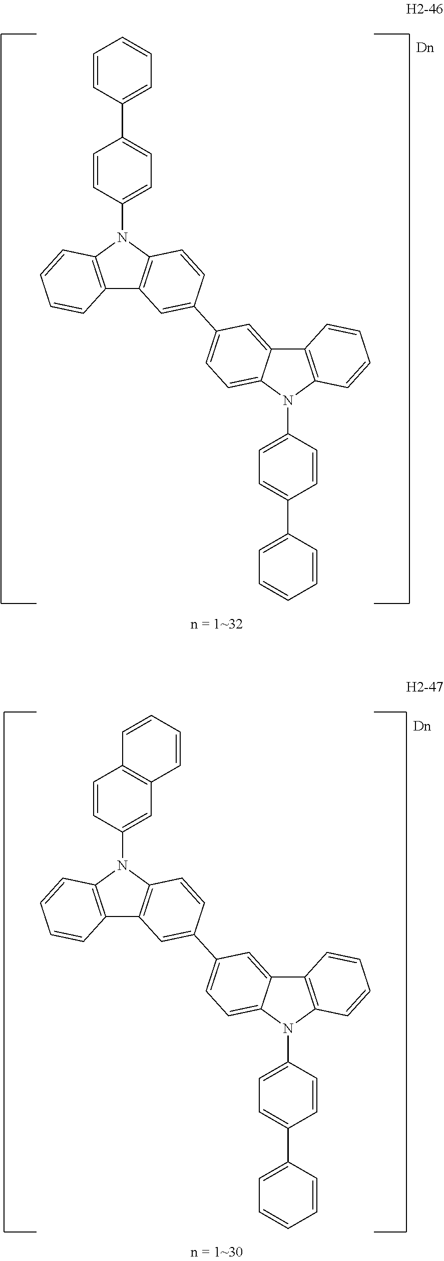 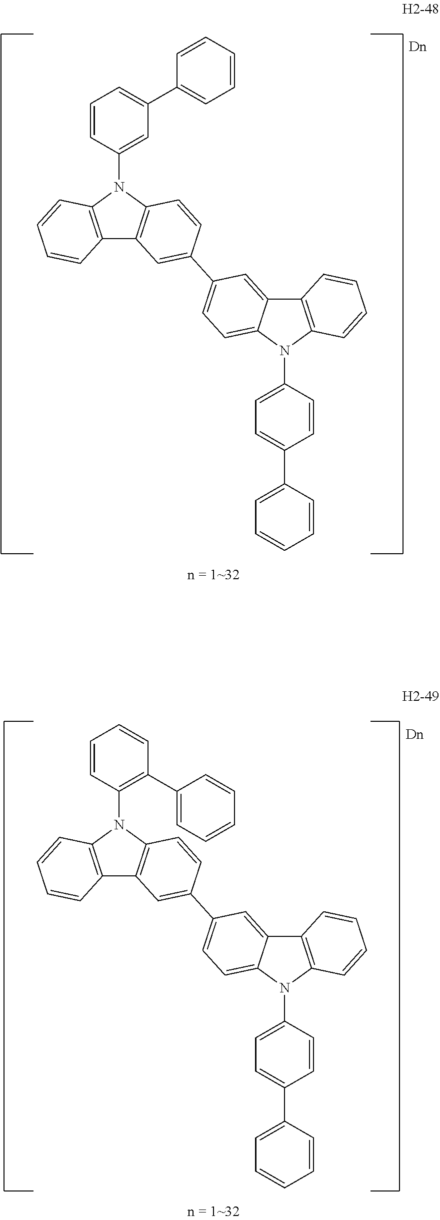 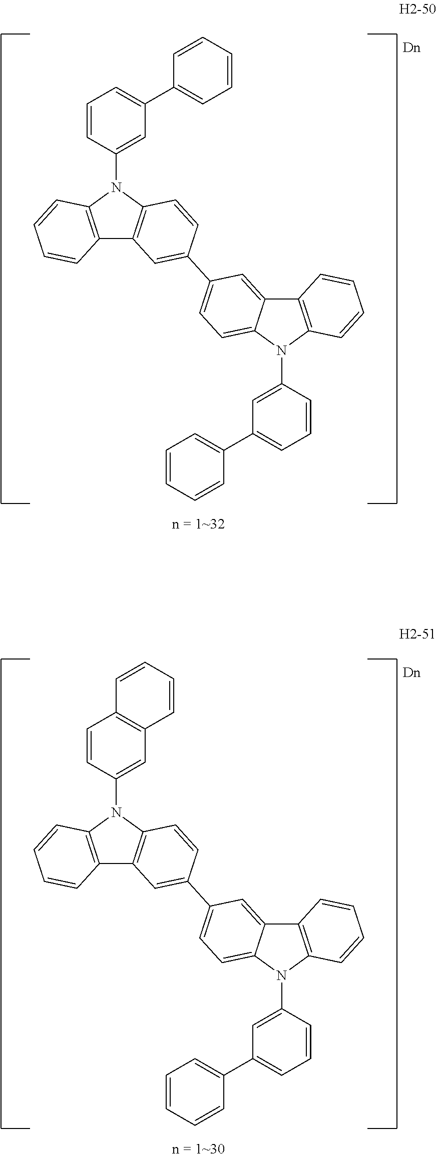 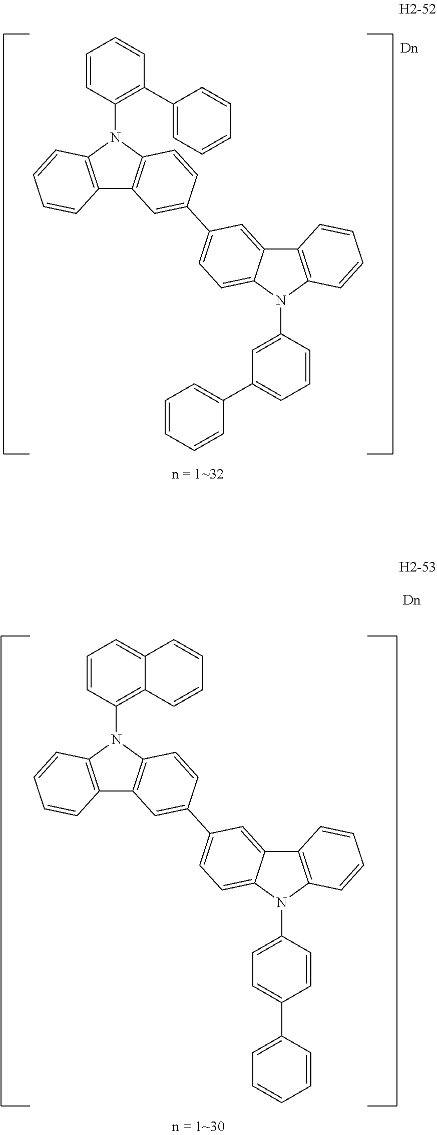 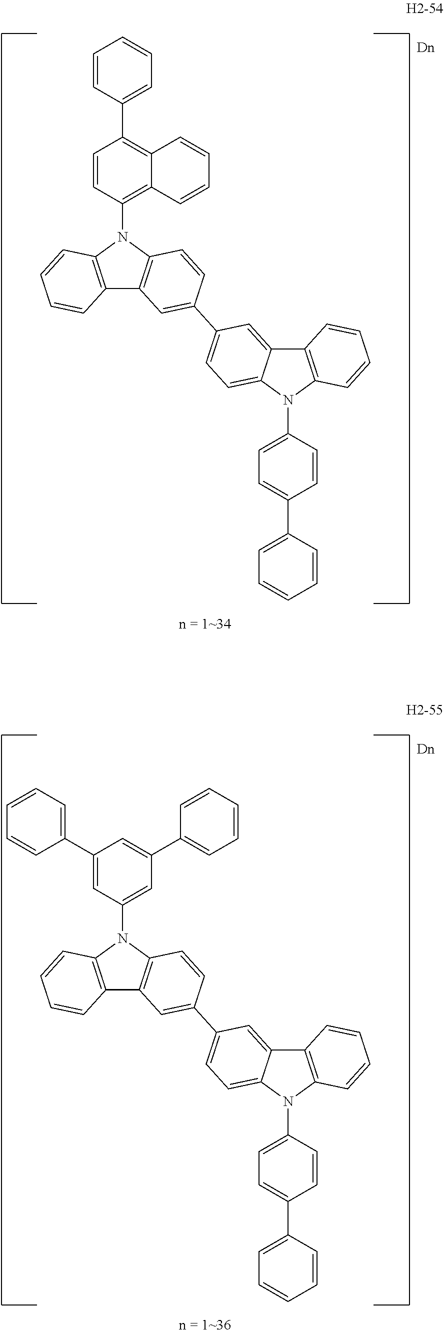 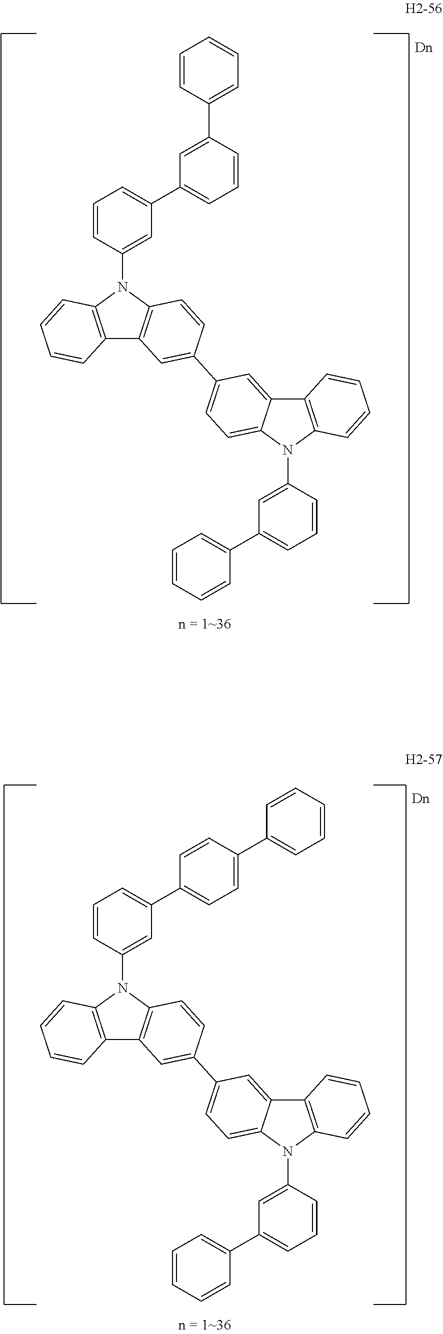 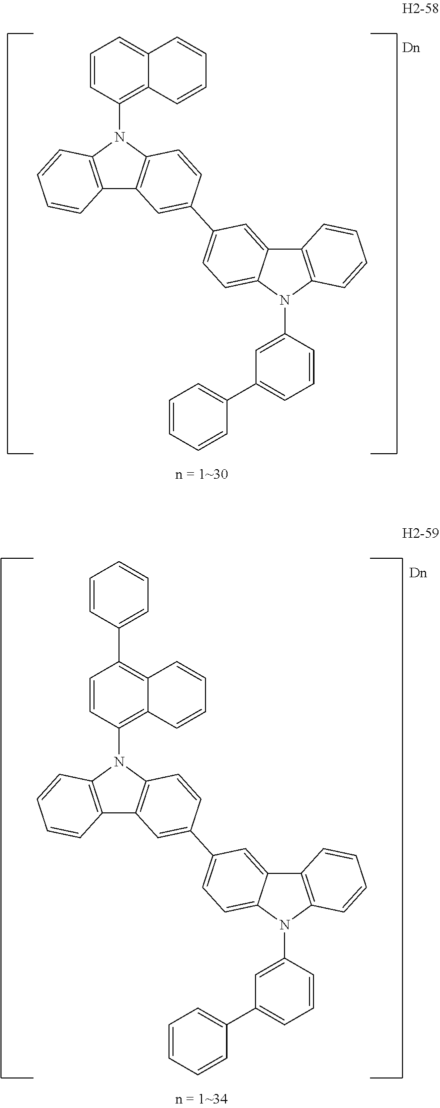 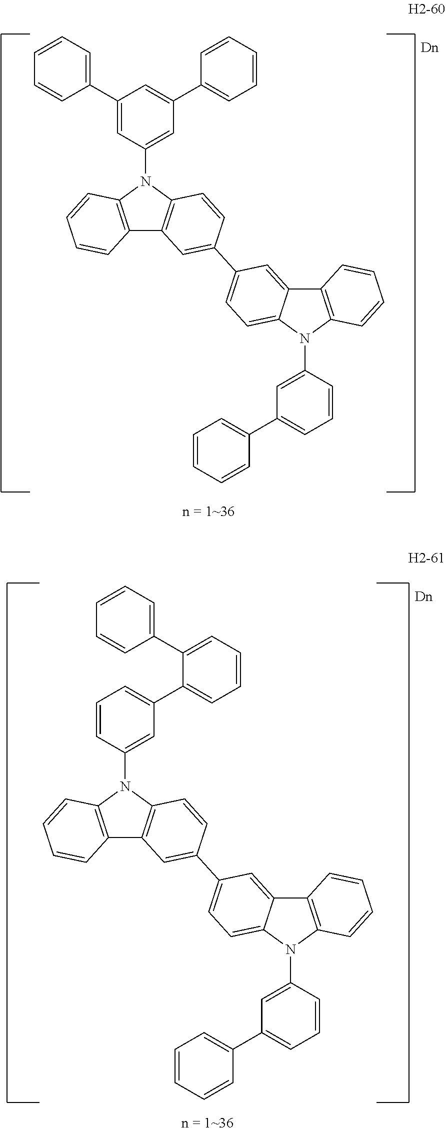 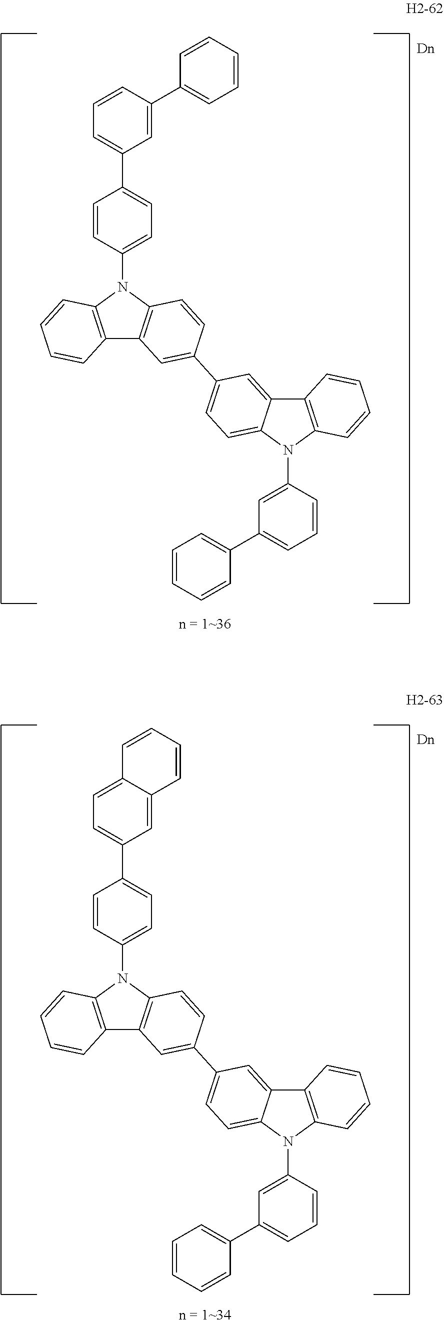 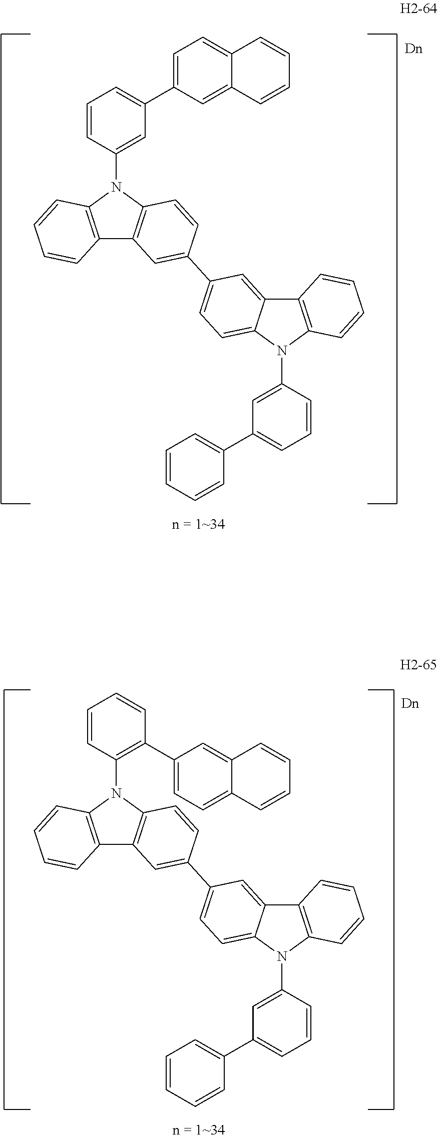 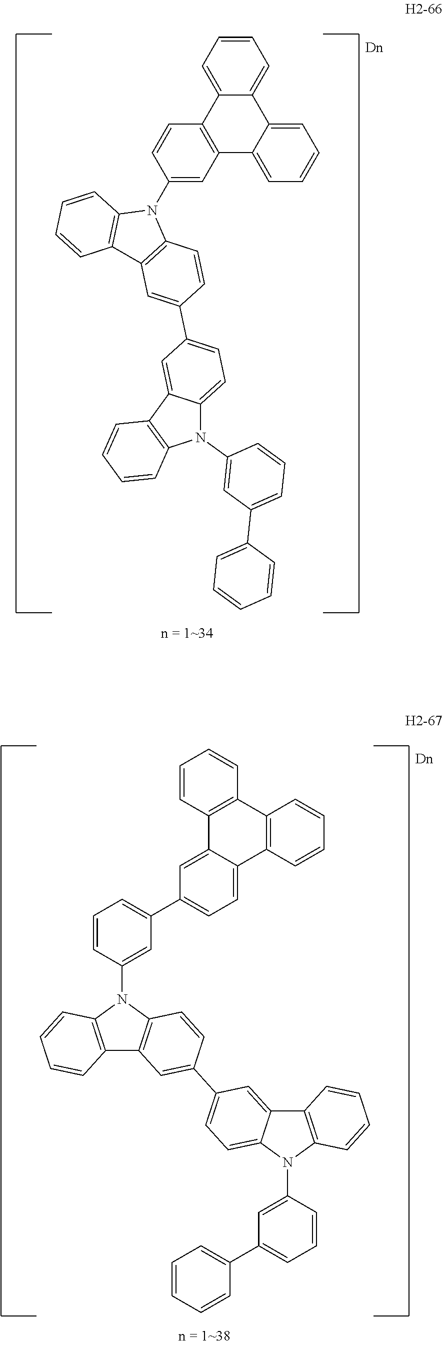 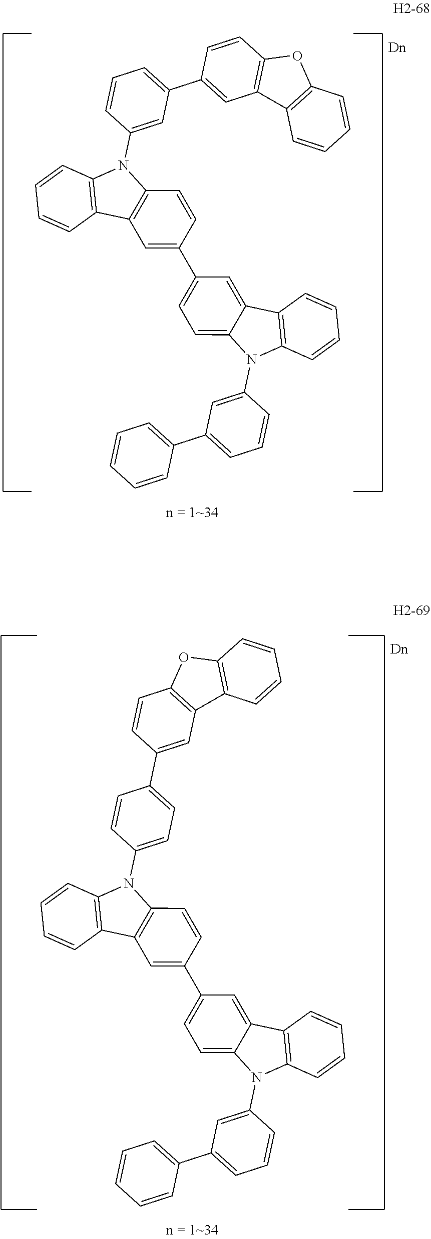 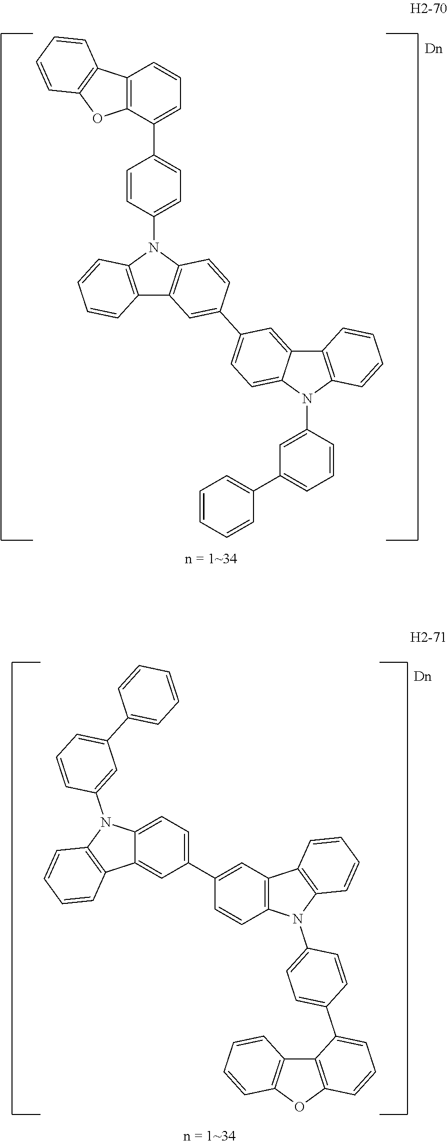 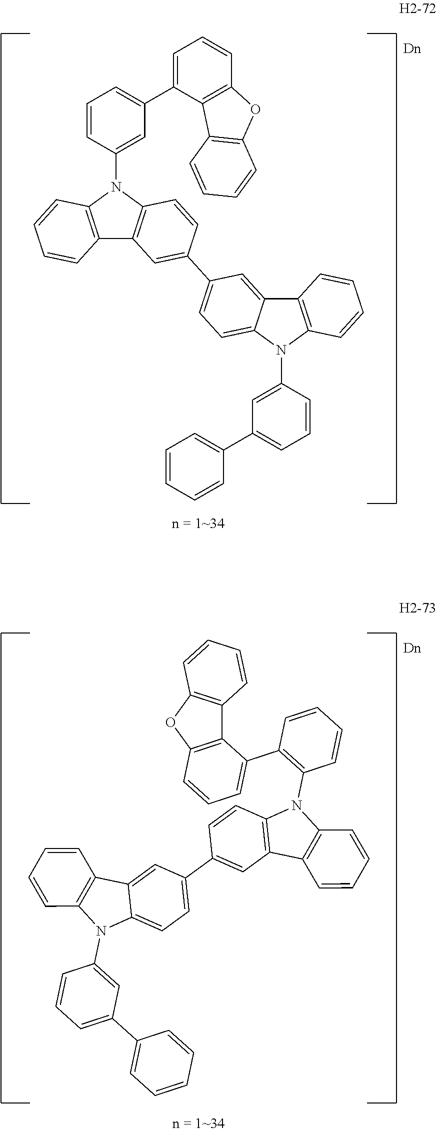 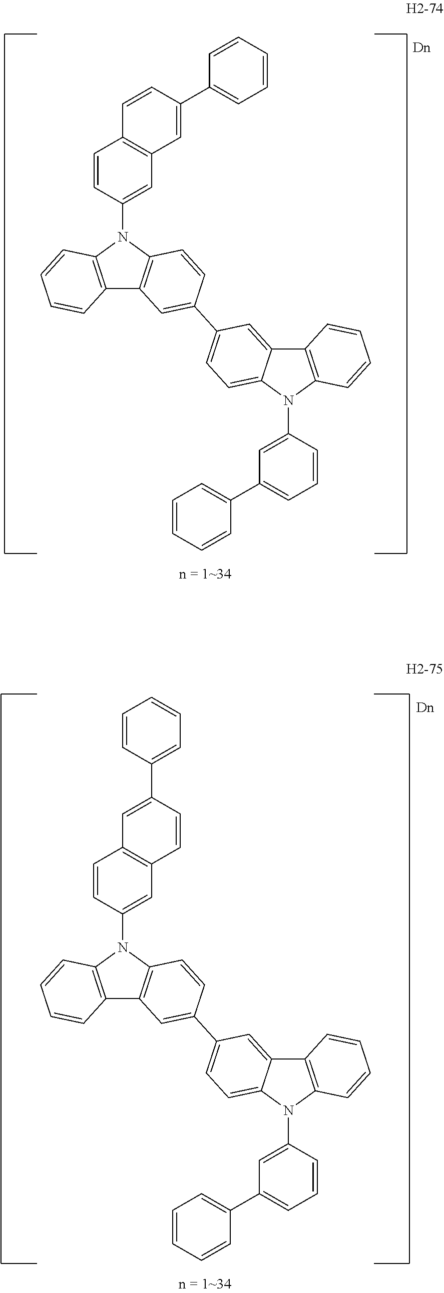 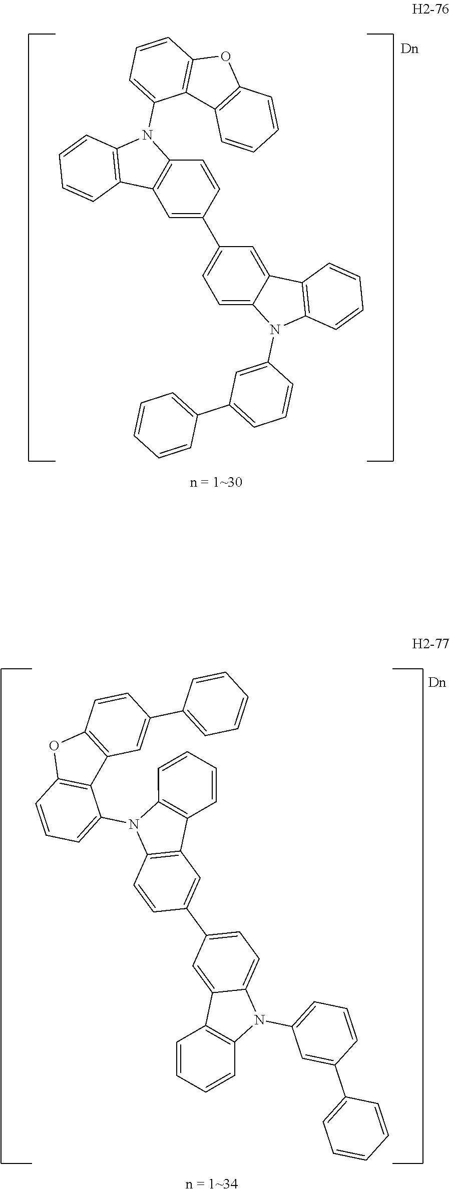 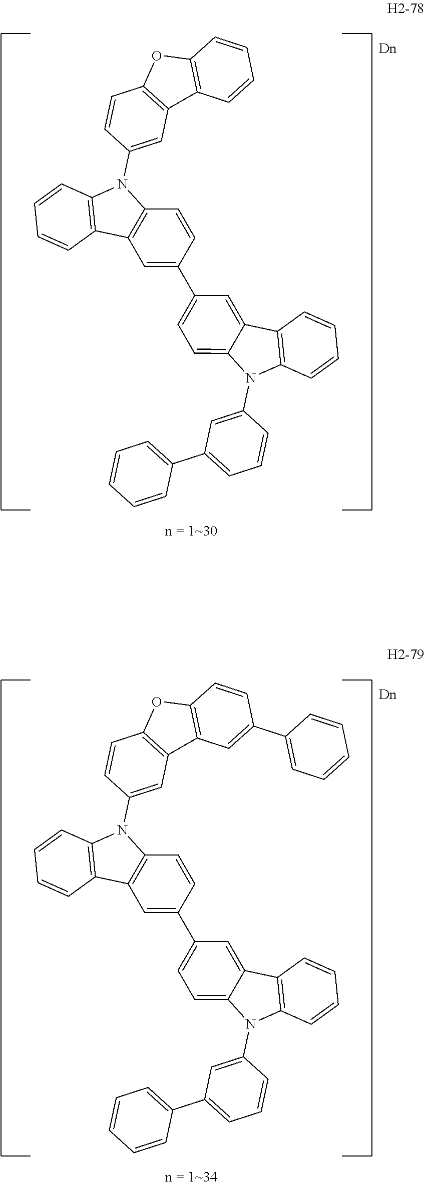 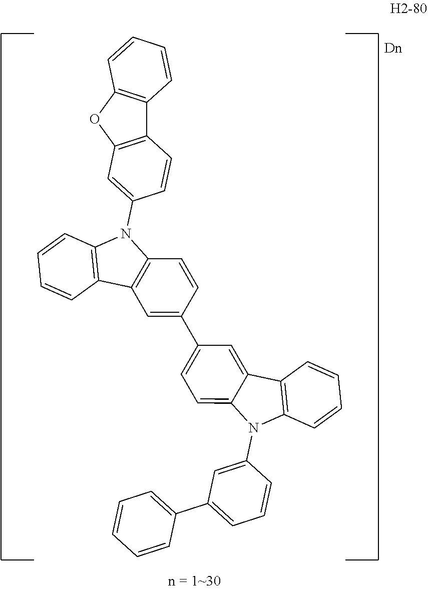 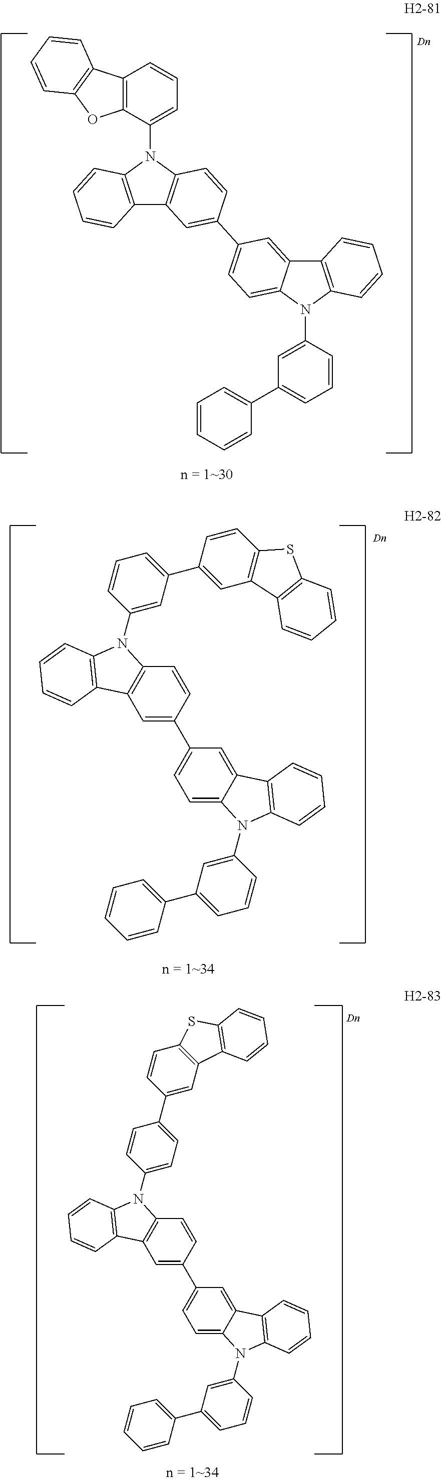 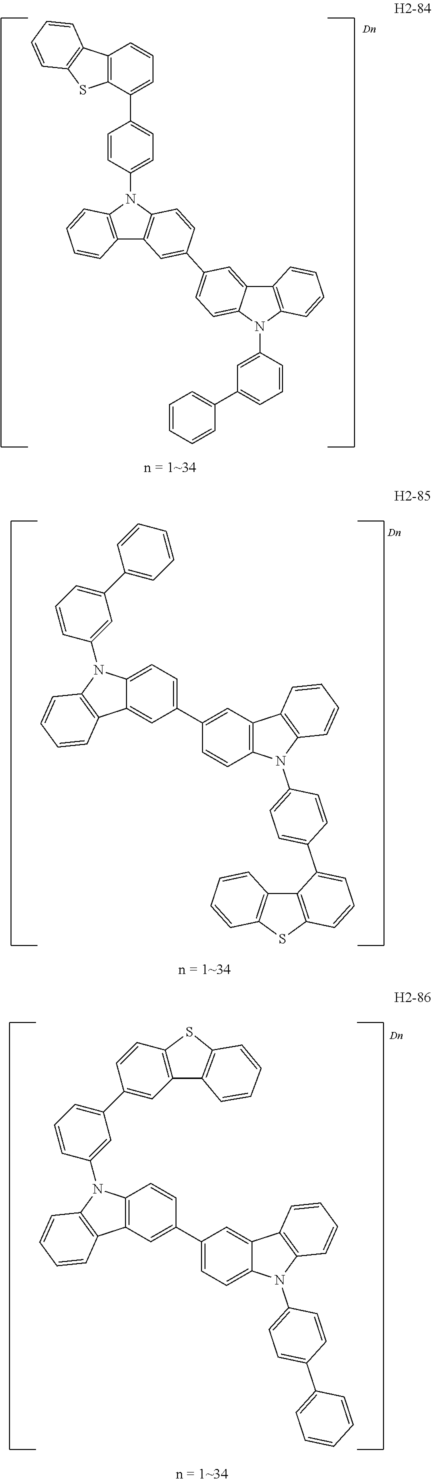 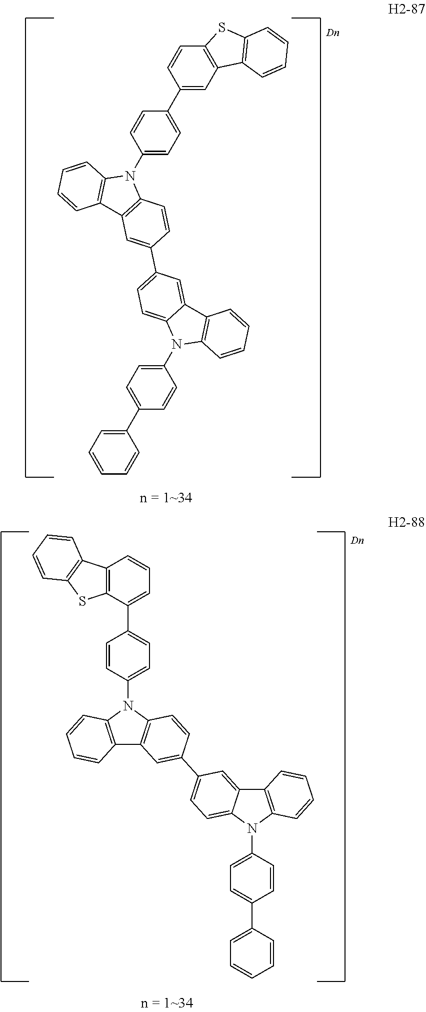 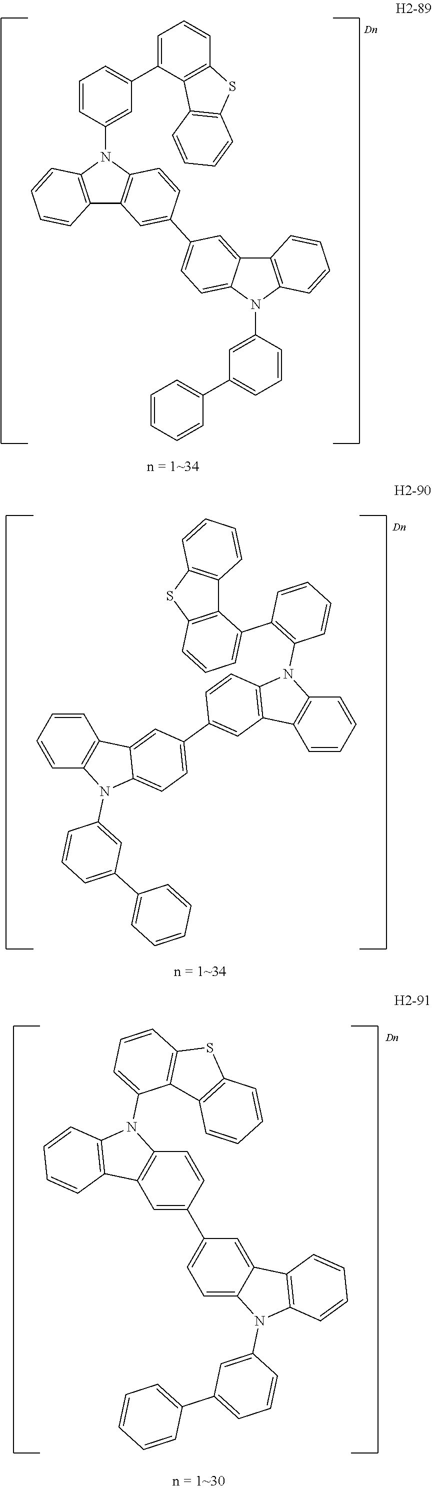 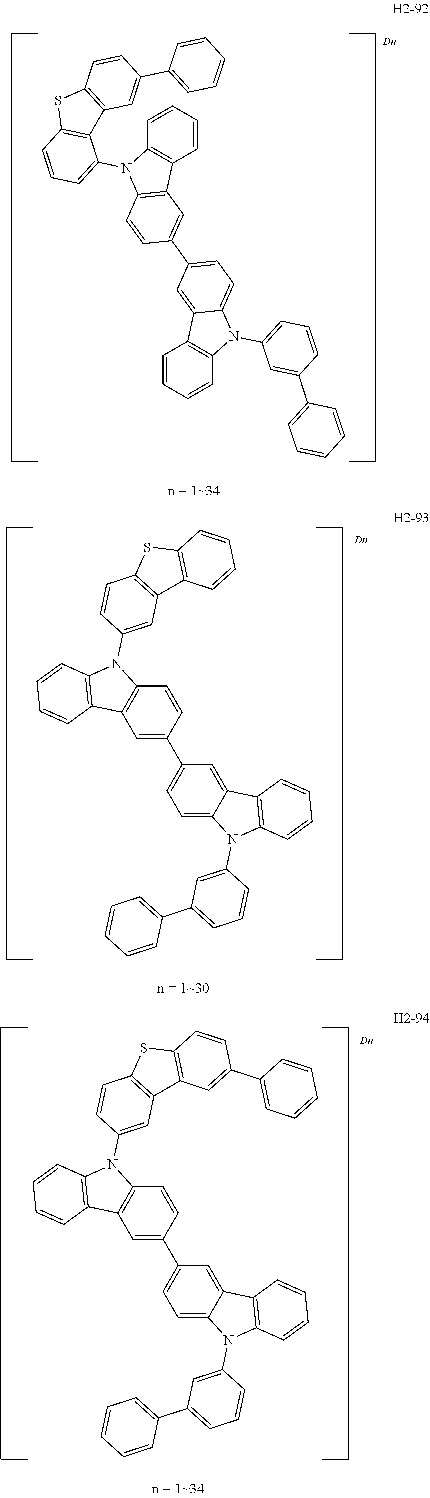 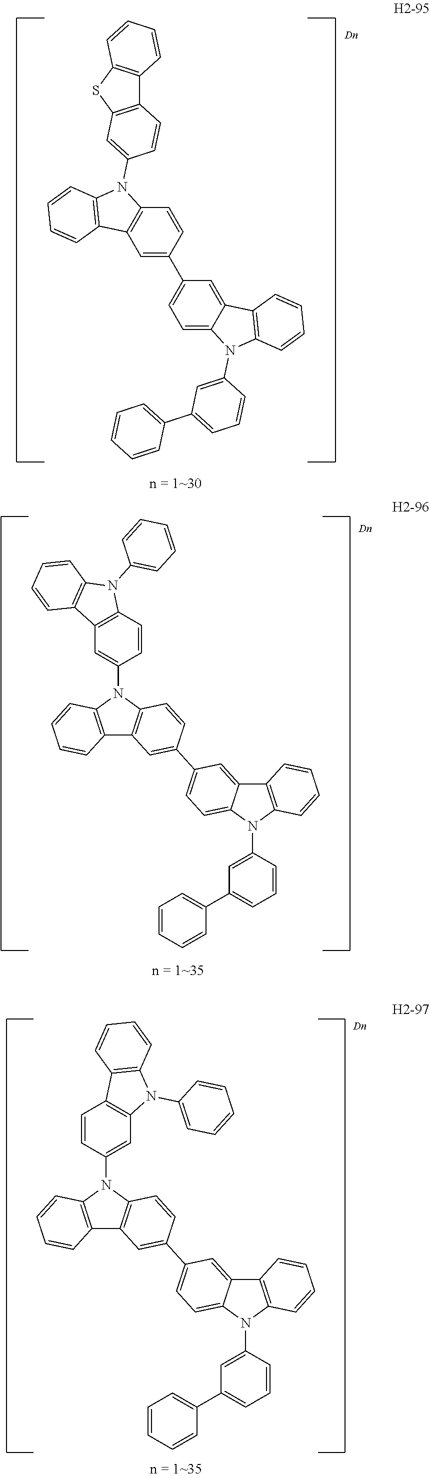 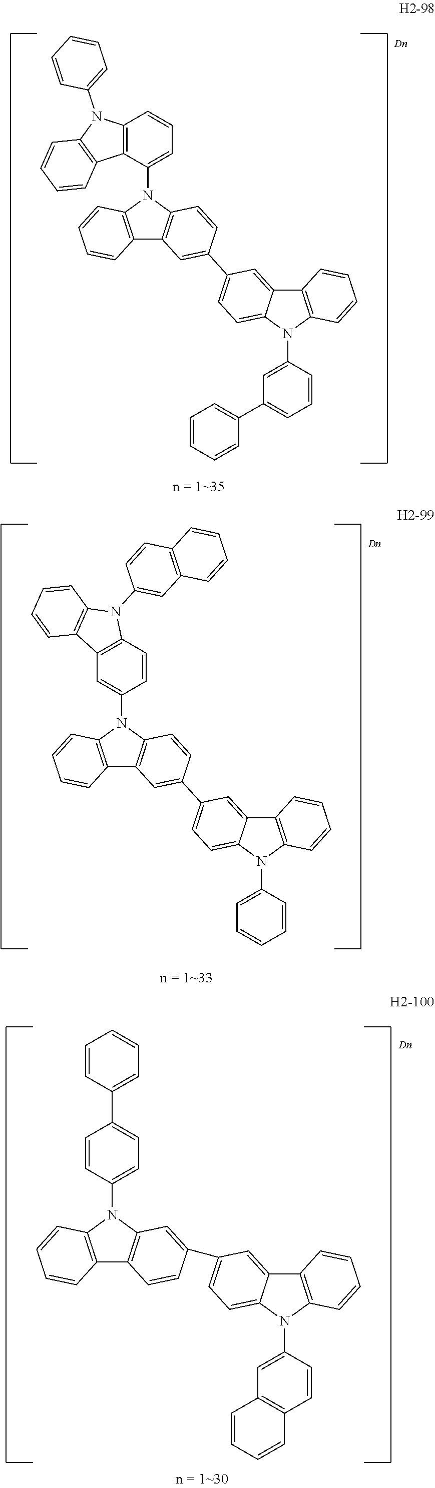 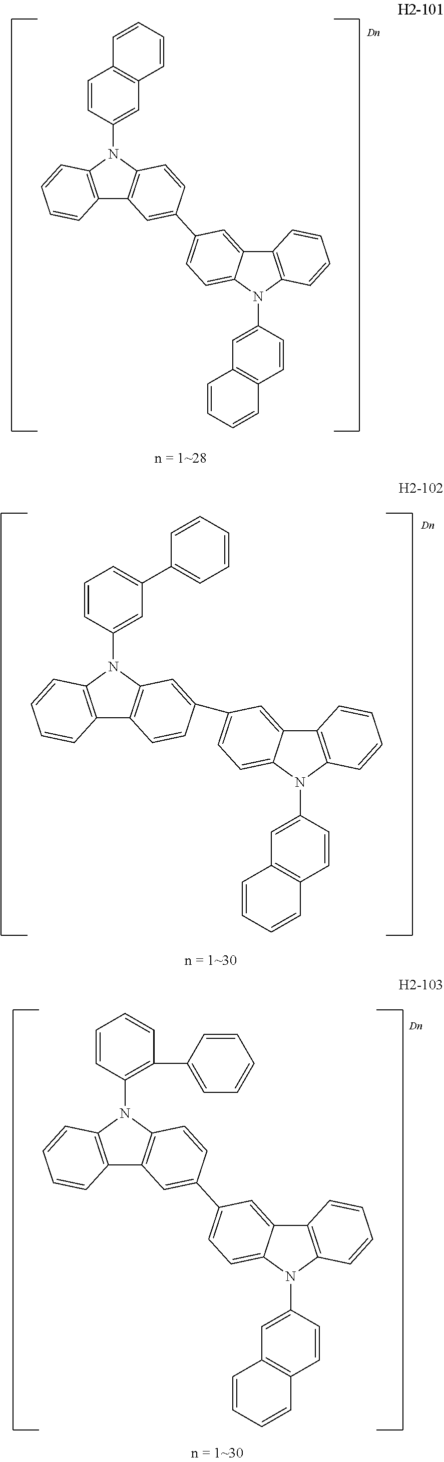 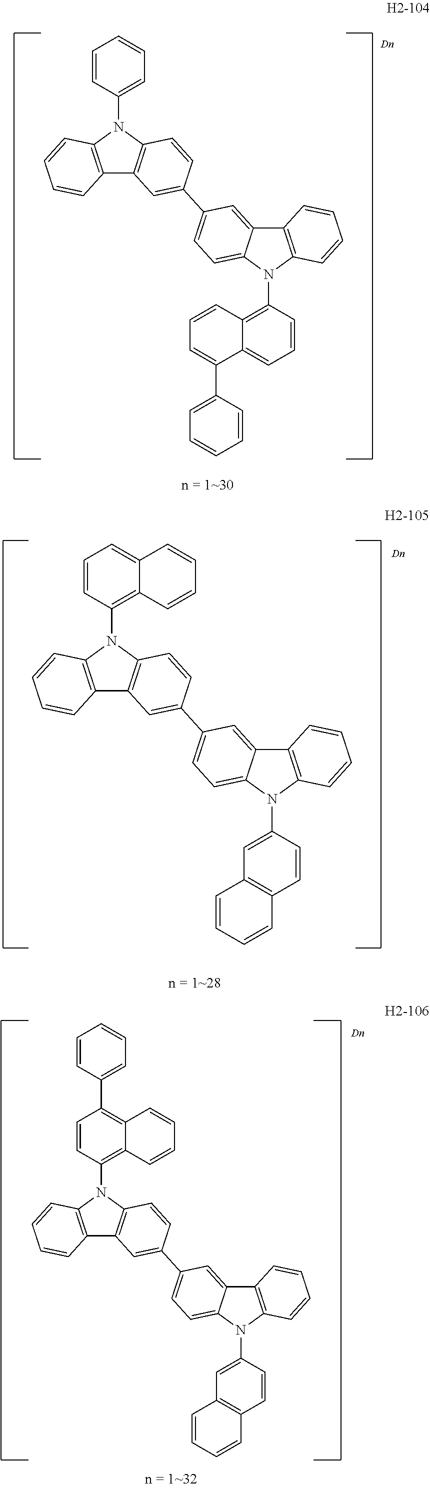 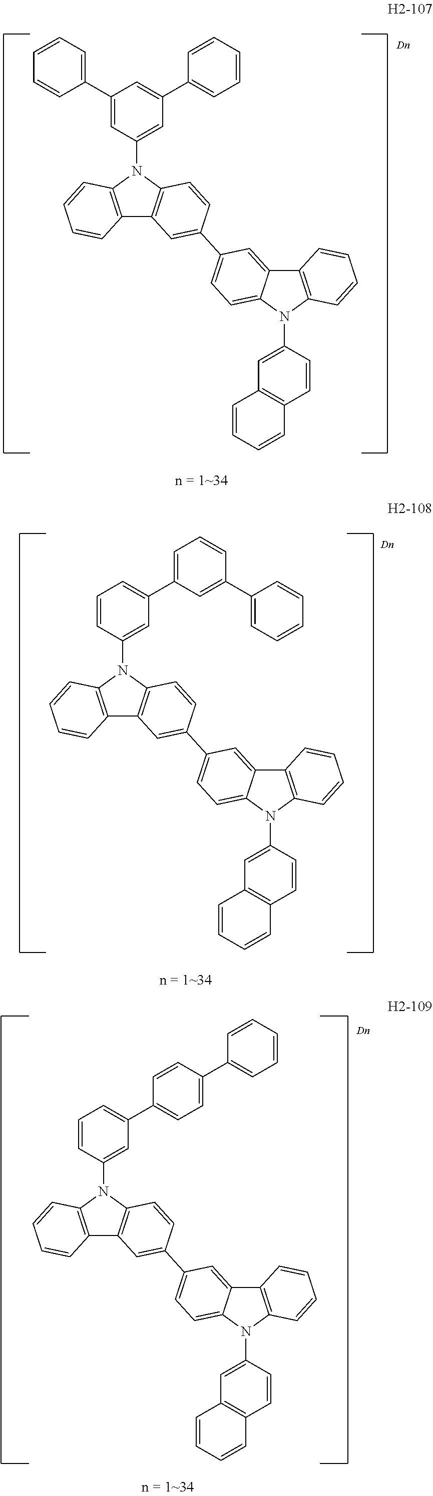 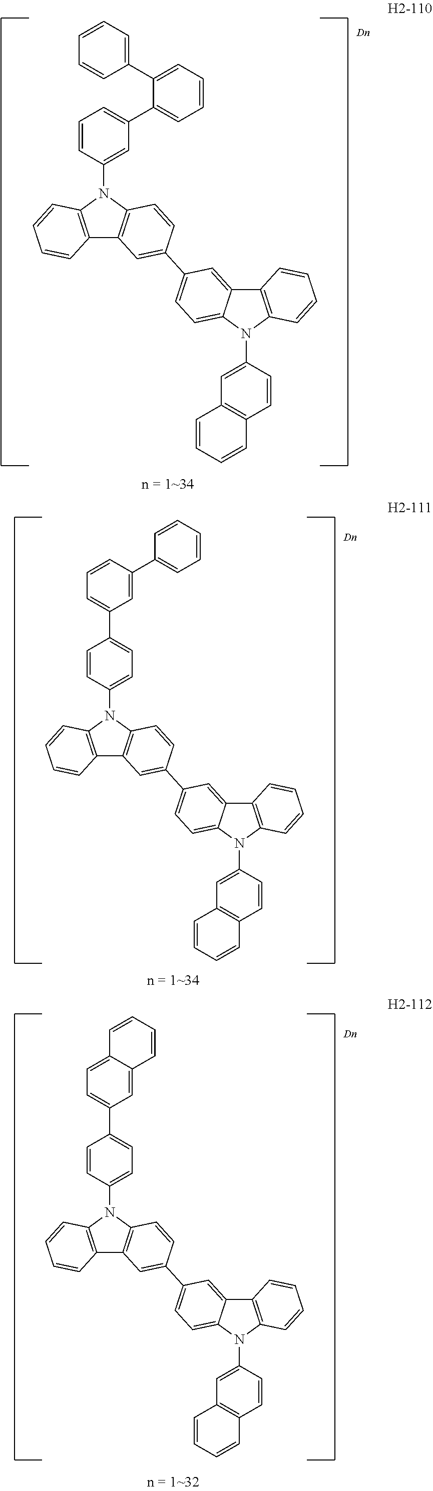 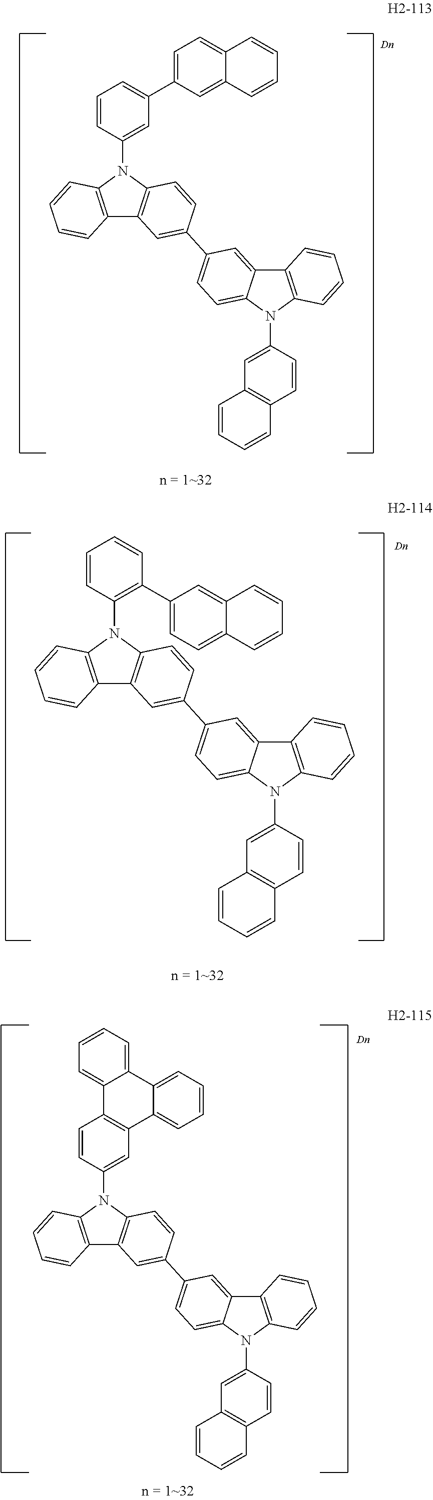 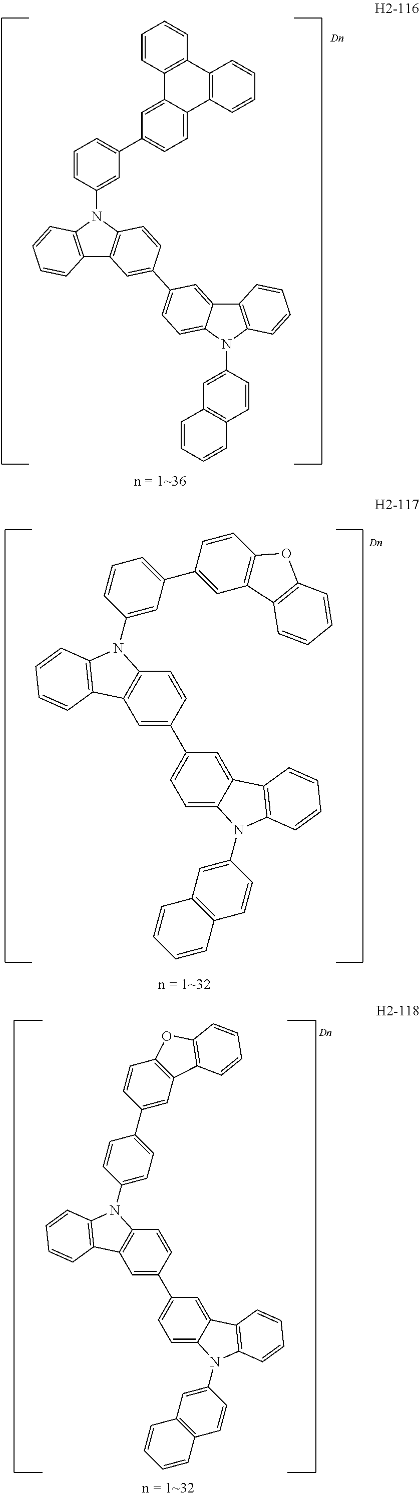 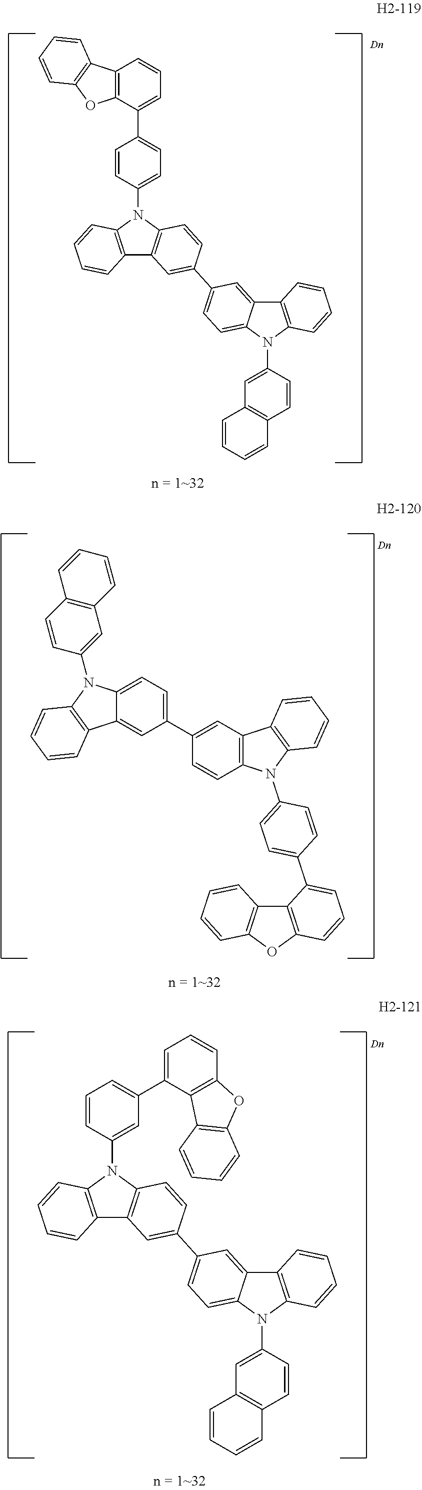 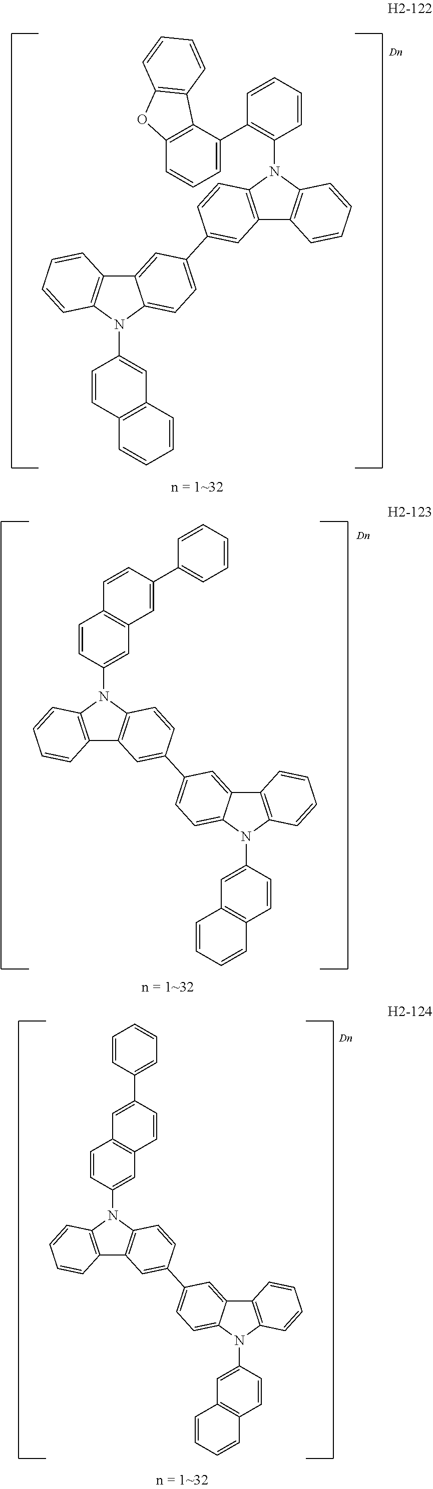 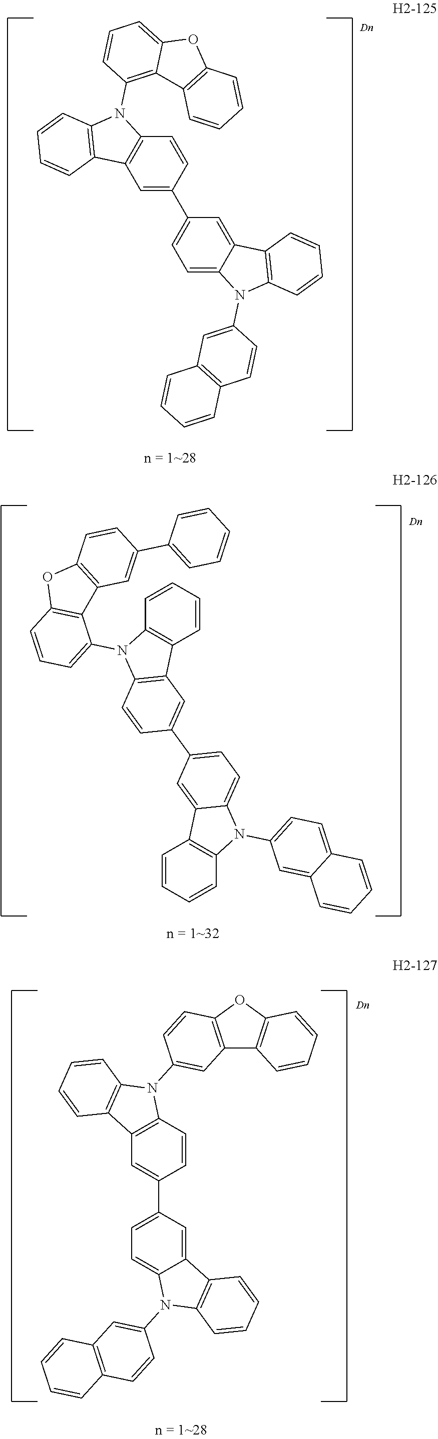 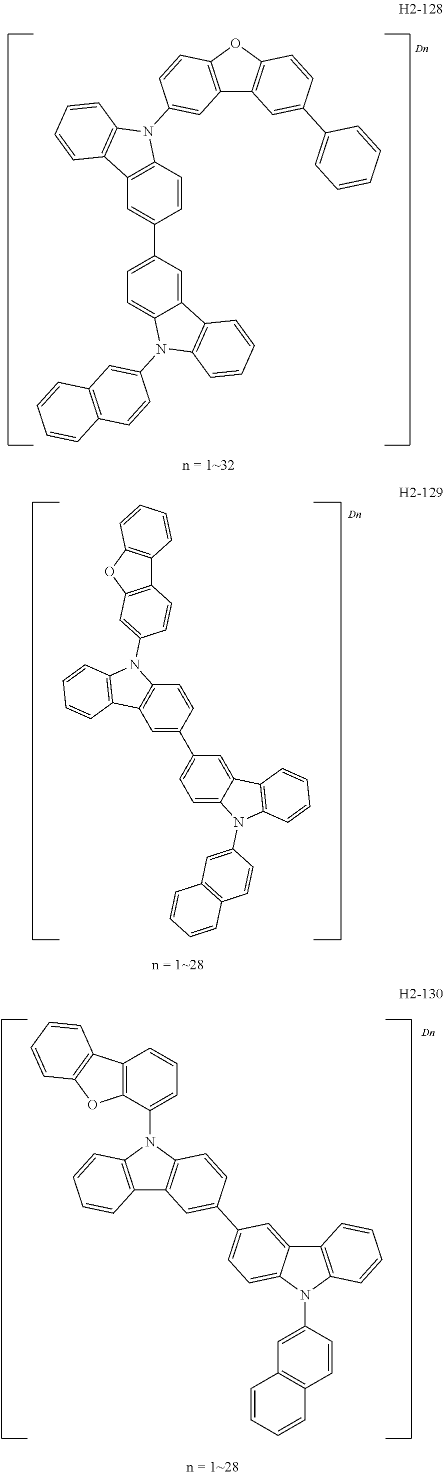 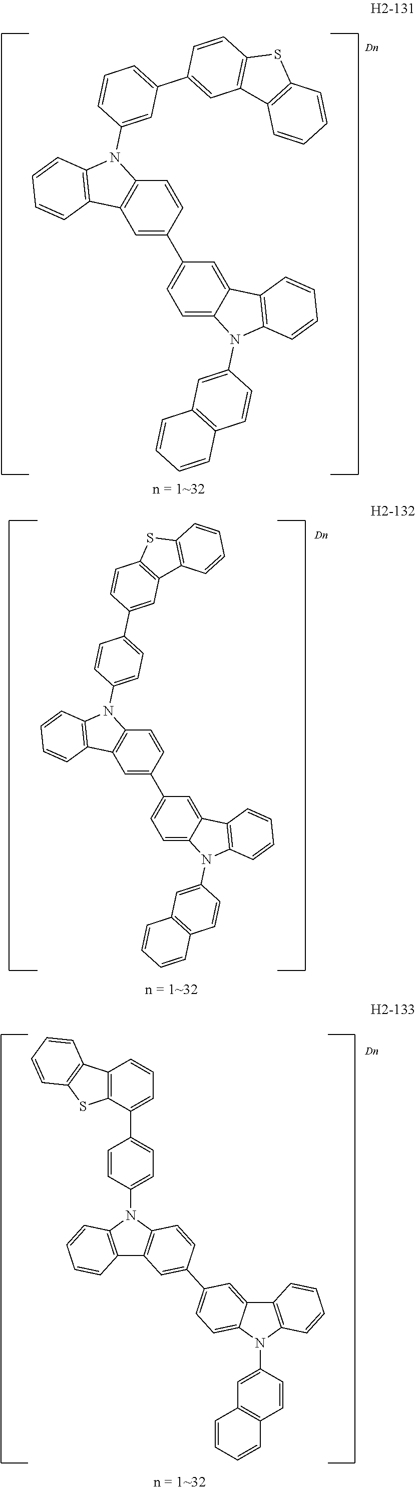 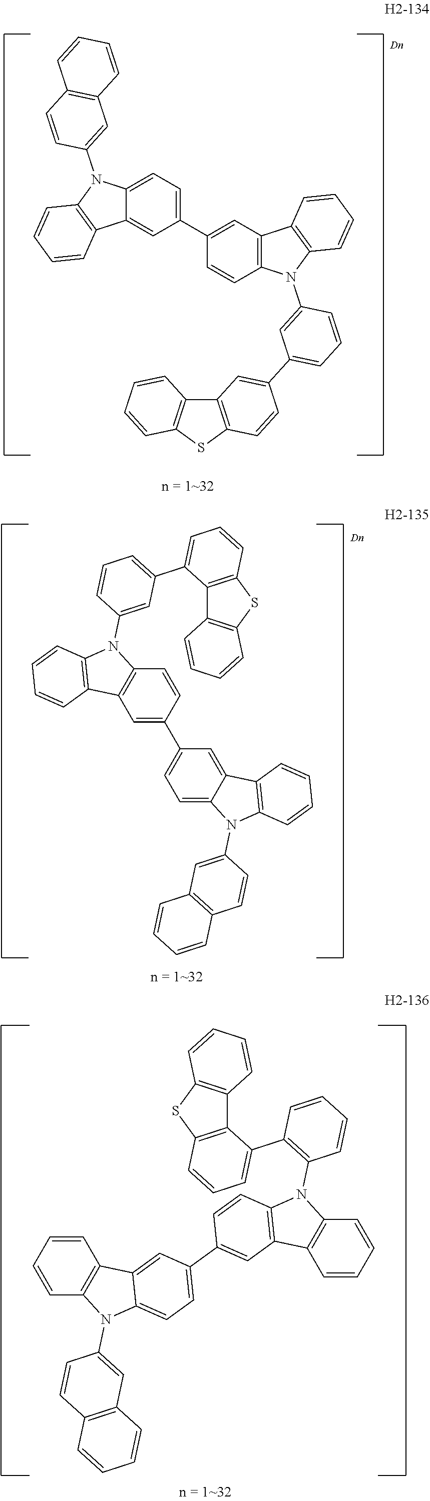 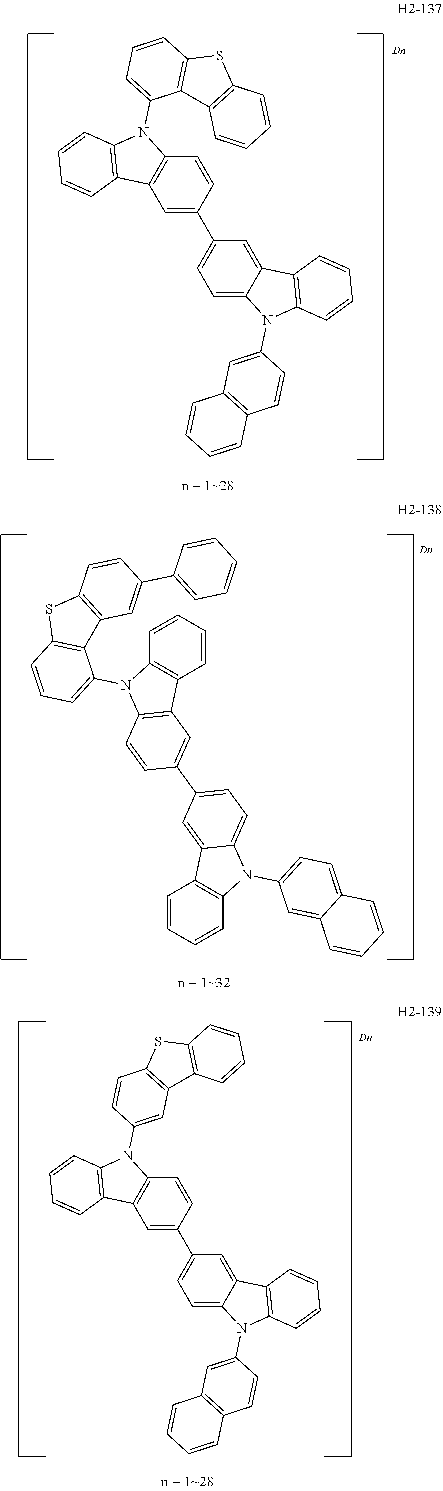 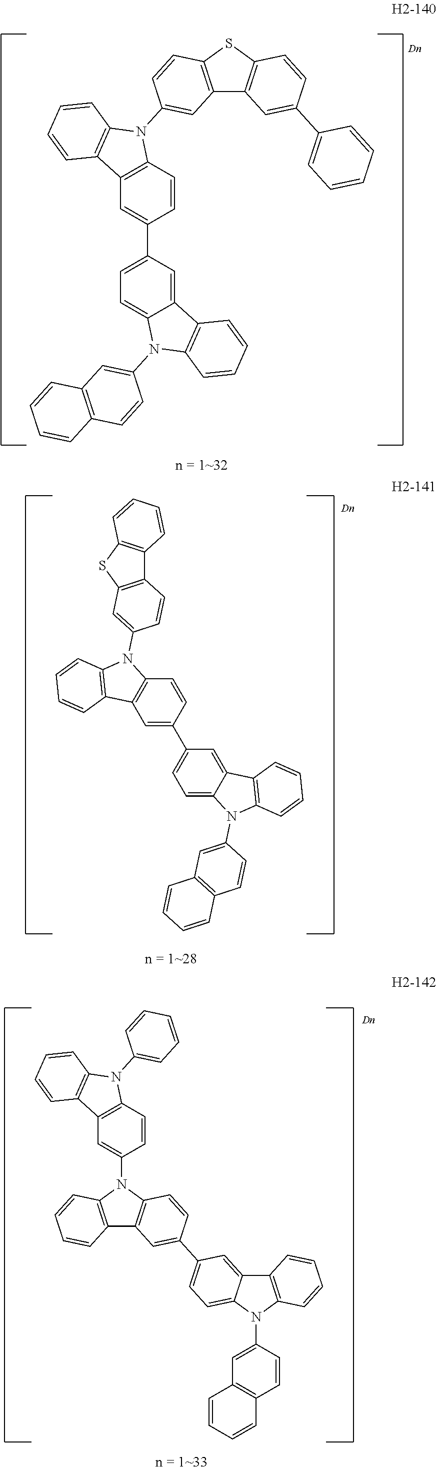 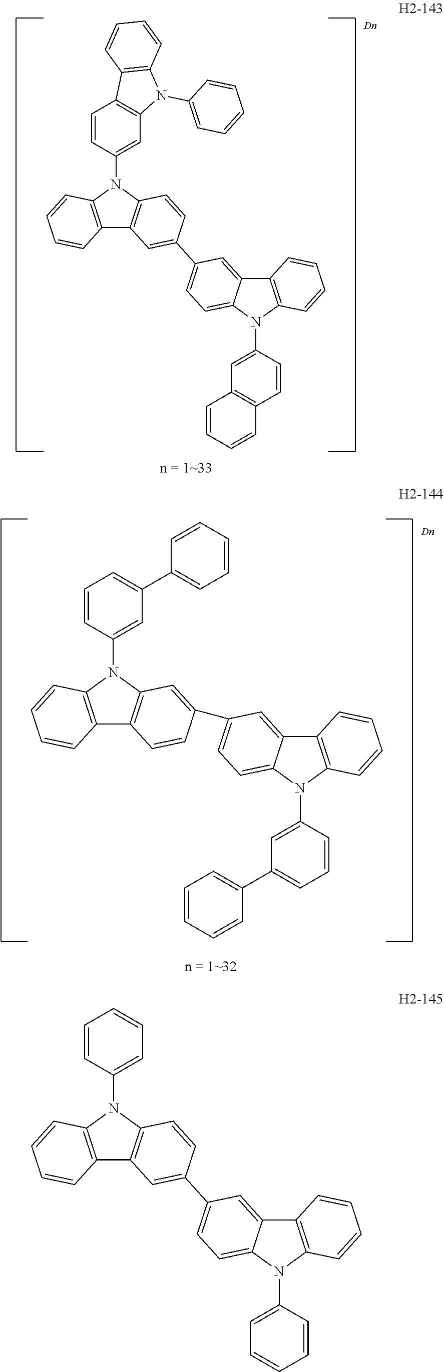 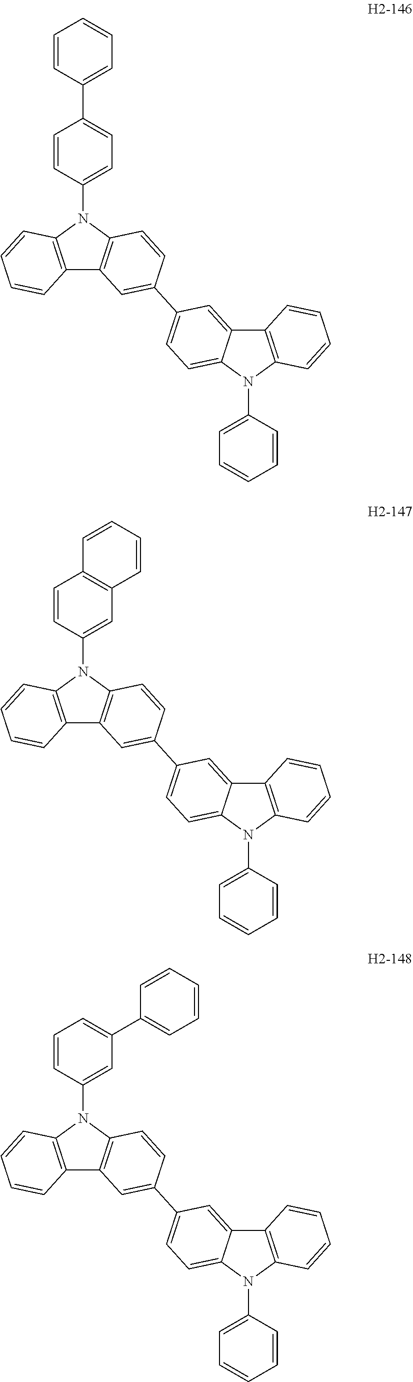 [SMILES: c1ccc(-c2ccc(-n3c4ccccc4c4cc(-c5ccc6c(c5)c5ccccc5n6-c5ccccc5)ccc43)cc2)cc1 ; c1ccc(-n2c3ccccc3c3cc(-c4ccc5c(c4)c4ccccc4n5-c4ccc5ccccc5c4)ccc32)cc1 ; c1ccc(-c2cccc(-n3c4ccccc4c4cc(-c5ccc6c(c5)c5ccccc5n6-c5ccccc5)ccc43)c2)cc1] 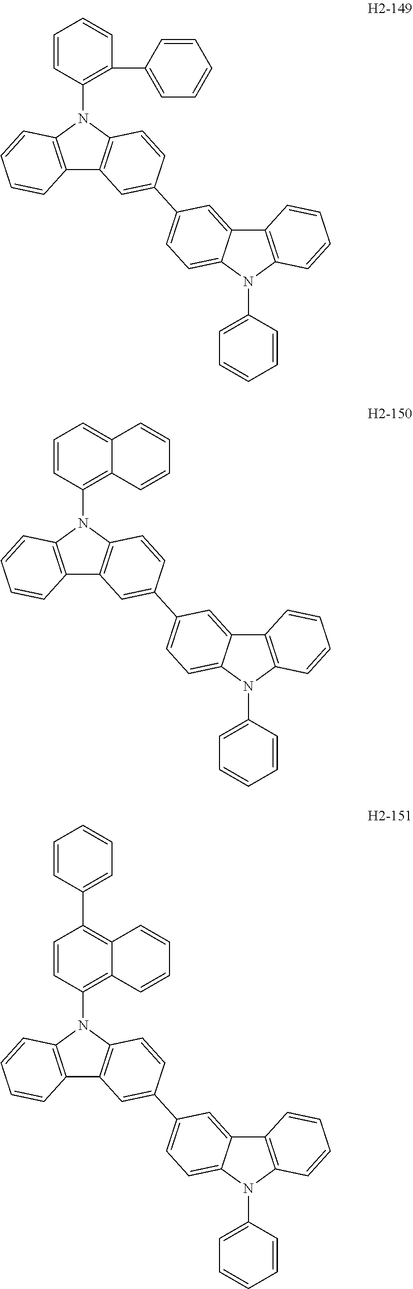 [SMILES: c1ccc(-c2ccccc2-n2c3ccccc3c3cc(-c4ccc5c(c4)c4ccccc4n5-c4ccccc4)ccc32)cc1 ; c1ccc(-n2c3ccccc3c3cc(-c4ccc5c(c4)c4ccccc4n5-c4cccc5ccccc45)ccc32)cc1 ; c1ccc(-c2ccc(-n3c4ccccc4c4cc(-c5ccc6c(c5)c5ccccc5n6-c5ccccc5)ccc43)c3ccccc23)cc1] 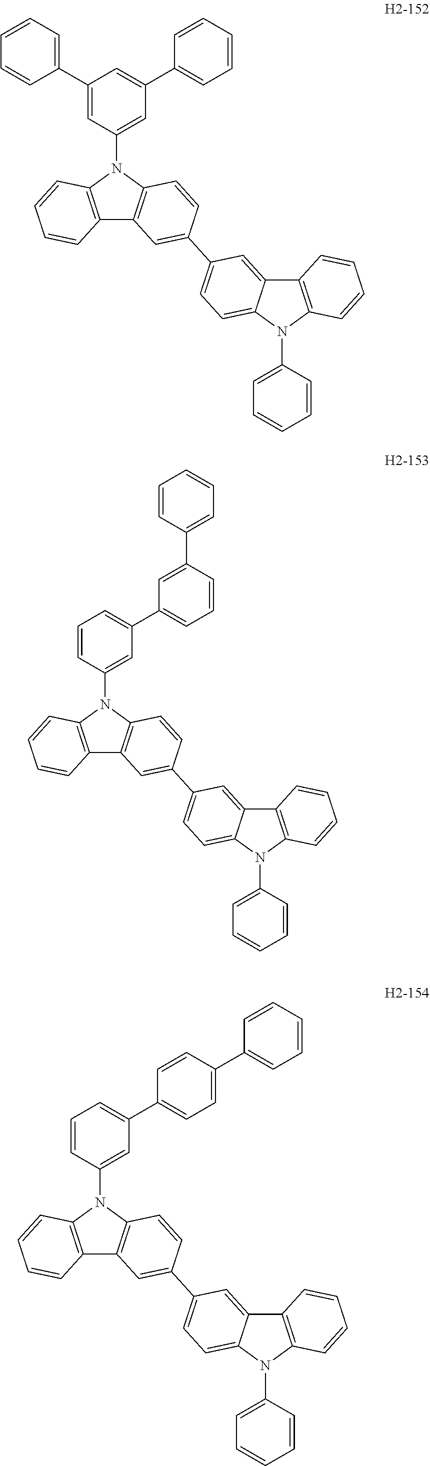 [SMILES: c1ccc(-c2cc(-c3ccccc3)cc(-n3c4ccccc4c4cc(-c5ccc6c(c5)c5ccccc5n6-c5ccccc5)ccc43)c2)cc1 ; c1ccc(-c2cccc(-c3cccc(-n4c5ccccc5c5cc(-c6ccc7c(c6)c6ccccc6n7-c6ccccc6)ccc54)c3)c2)cc1 ; c1ccc(-c2ccc(-c3cccc(-n4c5ccccc5c5cc(-c6ccc7c(c6)c6ccccc6n7-c6ccccc6)ccc54)c3)cc2)cc1] 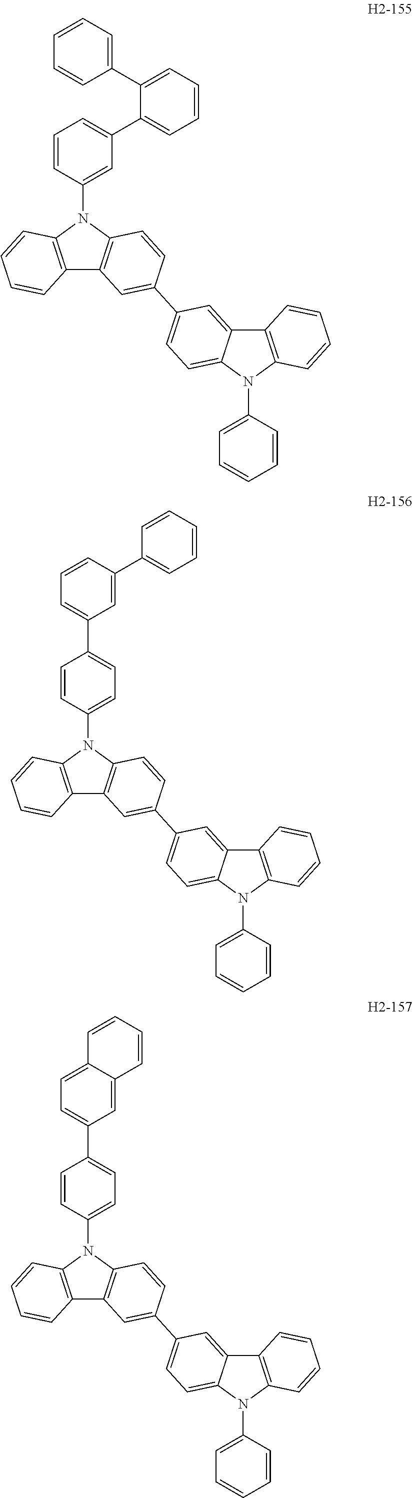 [SMILES: c1ccc(-c2ccccc2-c2cccc(-n3c4ccccc4c4cc(-c5ccc6c(c5)c5ccccc5n6-c5ccccc5)ccc43)c2)cc1 ; c1ccc(-c2cccc(-c3ccc(-n4c5ccccc5c5cc(-c6ccc7c(c6)c6ccccc6n7-c6ccccc6)ccc54)cc3)c2)cc1 ; c1ccc(-n2c3ccccc3c3cc(-c4ccc5c(c4)c4ccccc4n5-c4ccc(-c5ccc6ccccc6c5)cc4)ccc32)cc1] 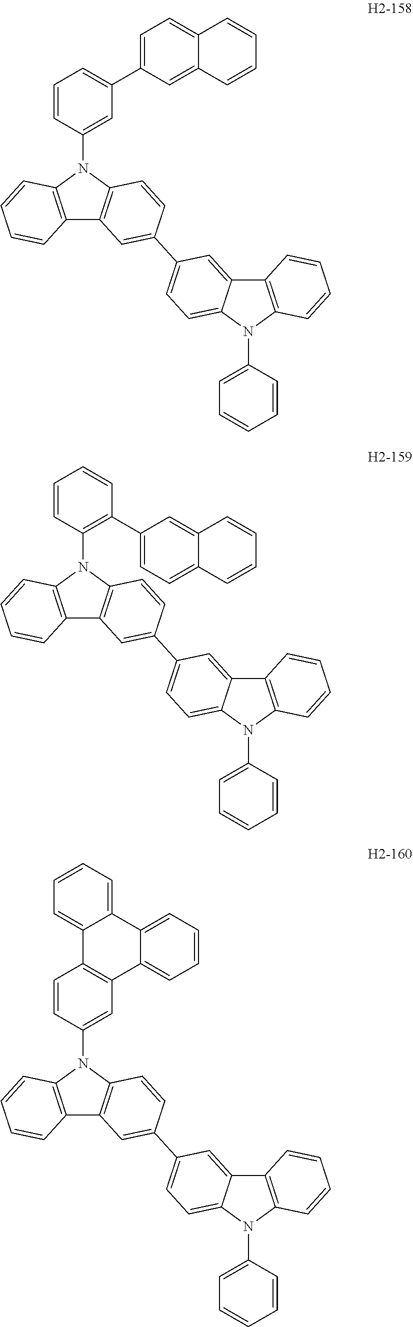 [SMILES: c1ccc(-n2c3ccccc3c3cc(-c4ccc5c(c4)c4ccccc4n5-c4cccc(-c5ccc6ccccc6c5)c4)ccc32)cc1 ; c1ccc(-n2c3ccccc3c3cc(-c4ccc5c(c4)c4ccccc4n5-c4ccccc4-c4ccc5ccccc5c4)ccc32)cc1 ; c1ccc(-n2c3ccccc3c3cc(-c4ccc5c(c4)c4ccccc4n5-c4ccc5c6ccccc6c6ccccc6c5c4)ccc32)cc1] 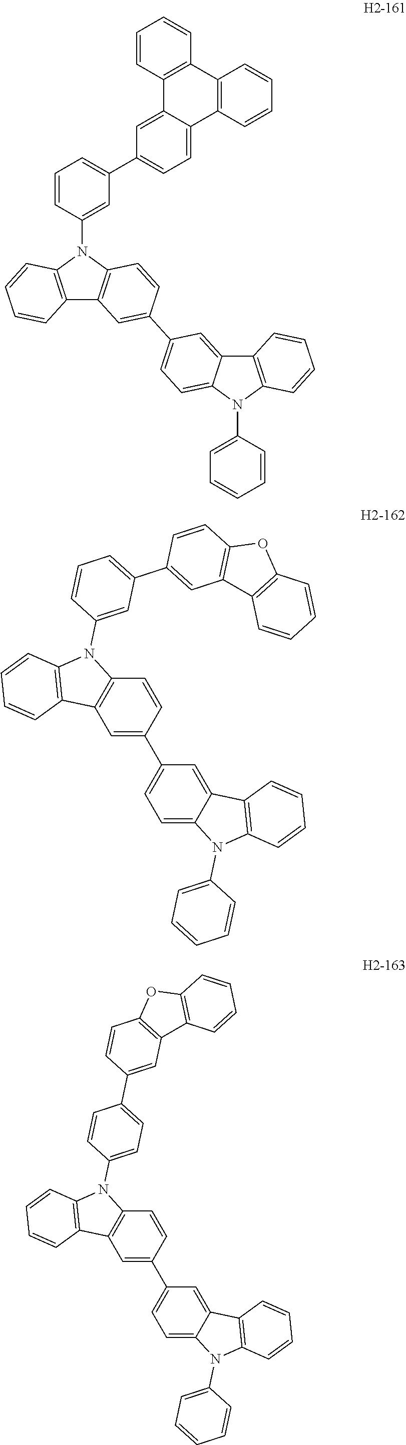 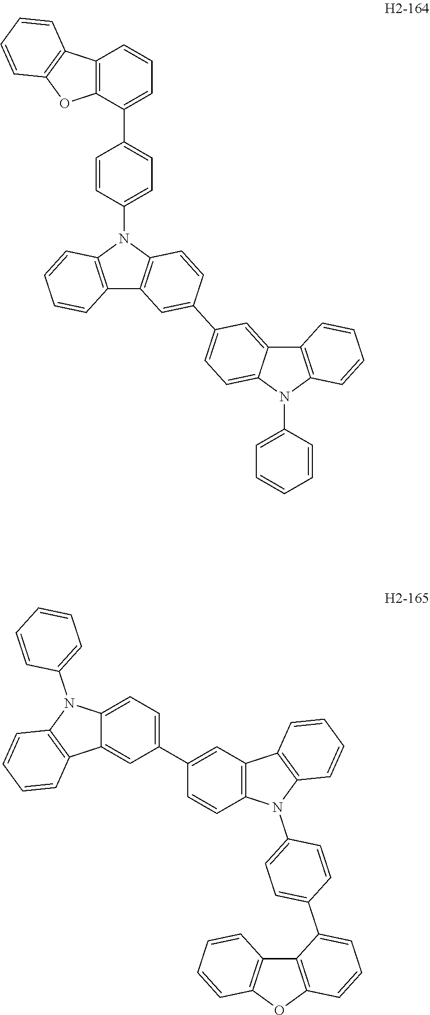 [SMILES: c1ccc(-n2c3ccccc3c3cc(-c4ccc5c(c4)c4ccccc4n5-c4ccc(-c5cccc6c5oc5ccccc56)cc4)ccc32)cc1 ; c1ccc(-n2c3ccccc3c3cc(-c4ccc5c(c4)c4ccccc4n5-c4ccc(-c5cccc6oc7ccccc7c56)cc4)ccc32)cc1] 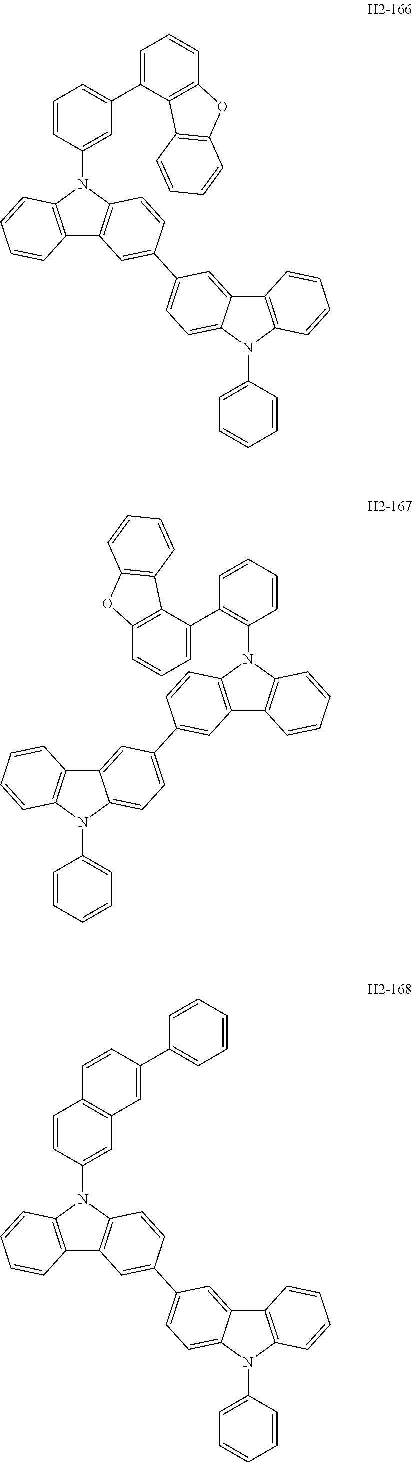 [SMILES: c1ccc(-n2c3ccccc3c3cc(-c4ccc5c(c4)c4ccccc4n5-c4cccc(-c5cccc6oc7ccccc7c56)c4)ccc32)cc1 ; c1ccc(-n2c3ccccc3c3cc(-c4ccc5c(c4)c4ccccc4n5-c4ccccc4-c4cccc5oc6ccccc6c45)ccc32)cc1 ; c1ccc(-c2ccc3ccc(-n4c5ccccc5c5cc(-c6ccc7c(c6)c6ccccc6n7-c6ccccc6)ccc54)cc3c2)cc1] 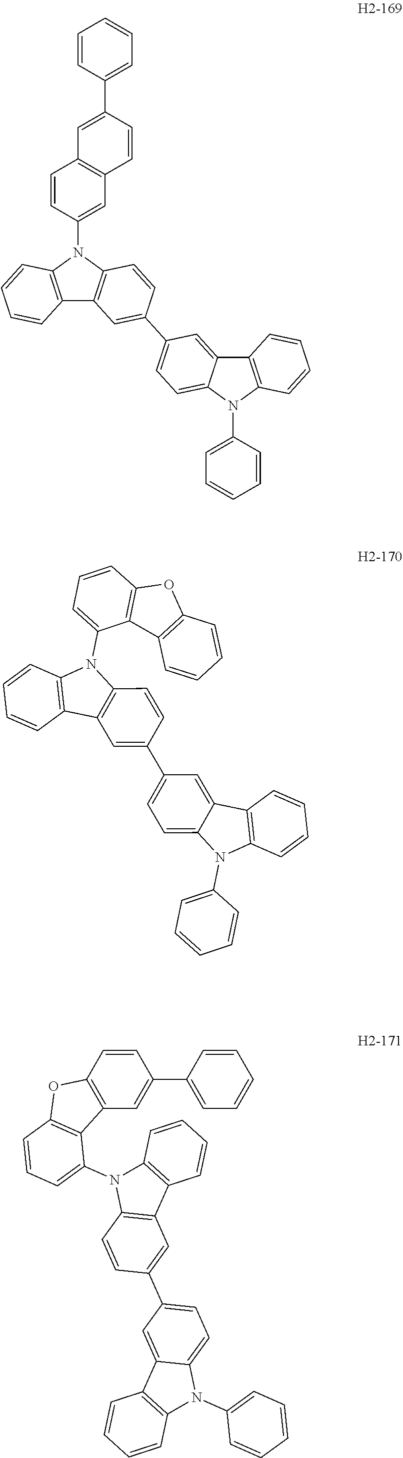 [SMILES: c1ccc(-c2ccc3cc(-n4c5ccccc5c5cc(-c6ccc7c(c6)c6ccccc6n7-c6ccccc6)ccc54)ccc3c2)cc1 ; c1ccc(-n2c3ccccc3c3cc(-c4ccc5c(c4)c4ccccc4n5-c4cccc5oc6ccccc6c45)ccc32)cc1 ; c1ccc(-c2ccc3oc4cccc(-n5c6ccccc6c6cc(-c7ccc8c(c7)c7ccccc7n8-c7ccccc7)ccc65)c4c3c2)cc1] 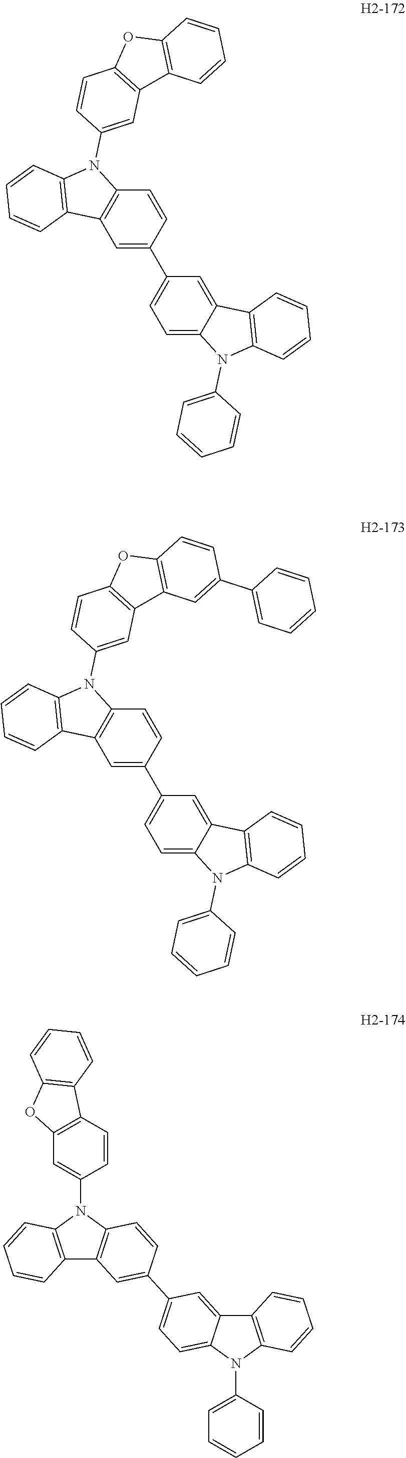 [SMILES: c1ccc(-n2c3ccccc3c3cc(-c4ccc5c(c4)c4ccccc4n5-c4ccc5oc6ccccc6c5c4)ccc32)cc1 ; c1ccc(-c2ccc3oc4ccc(-n5c6ccccc6c6cc(-c7ccc8c(c7)c7ccccc7n8-c7ccccc7)ccc65)cc4c3c2)cc1 ; c1ccc(-n2c3ccccc3c3cc(-c4ccc5c(c4)c4ccccc4n5-c4ccc5c(c4)oc4ccccc45)ccc32)cc1] 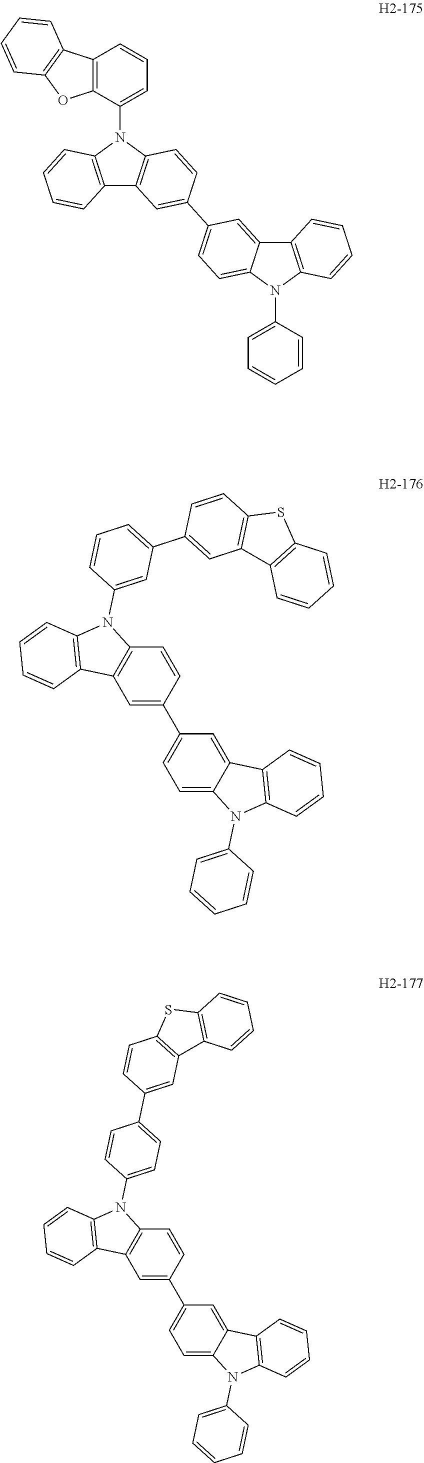 [SMILES: c1ccc(-n2c3ccccc3c3cc(-c4ccc5c(c4)c4ccccc4n5-c4cccc5c4oc4ccccc45)ccc32)cc1 ; c1ccc(-n2c3ccccc3c3cc(-c4ccc5c(c4)c4ccccc4n5-c4cccc(-c5ccc6sc7ccccc7c6c5)c4)ccc32)cc1 ; c1ccc(-n2c3ccccc3c3cc(-c4ccc5c(c4)c4ccccc4n5-c4ccc(-c5ccc6sc7ccccc7c6c5)cc4)ccc32)cc1] 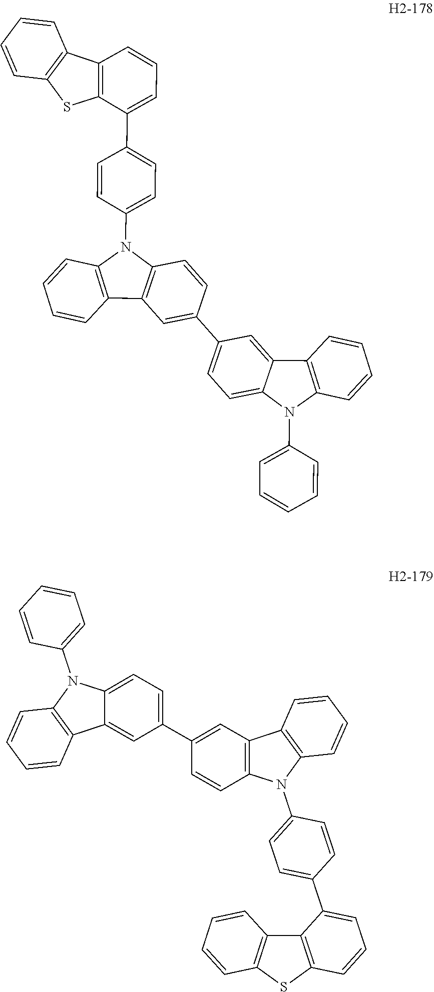 [SMILES: c1ccc(-n2c3ccccc3c3cc(-c4ccc5c(c4)c4ccccc4n5-c4ccc(-c5cccc6c5sc5ccccc56)cc4)ccc32)cc1 ; c1ccc(-n2c3ccccc3c3cc(-c4ccc5c(c4)c4ccccc4n5-c4ccc(-c5cccc6sc7ccccc7c56)cc4)ccc32)cc1] 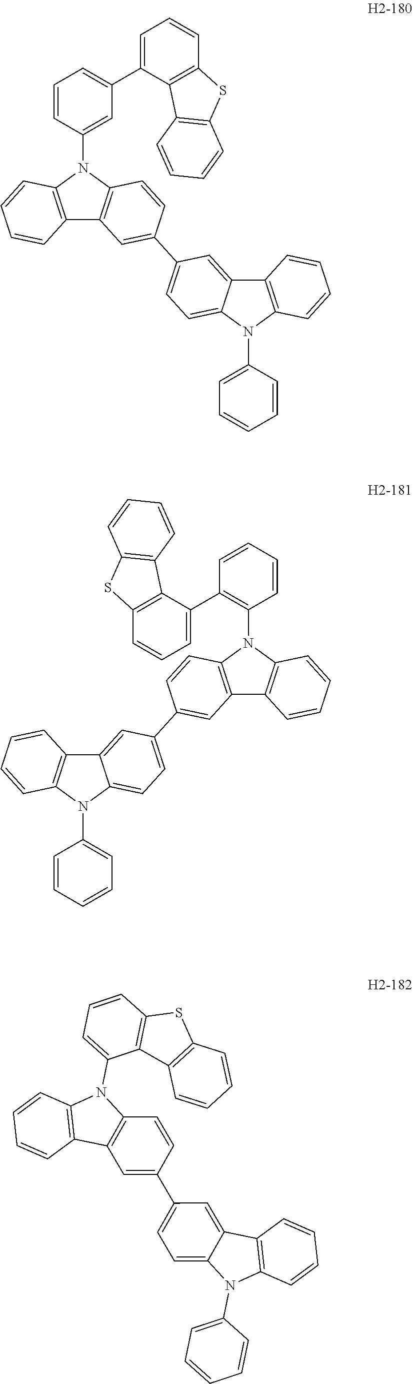 [SMILES: c1ccc(-n2c3ccccc3c3cc(-c4ccc5c(c4)c4ccccc4n5-c4cccc(-c5cccc6sc7ccccc7c56)c4)ccc32)cc1 ; c1ccc(-n2c3ccccc3c3cc(-c4ccc5c(c4)c4ccccc4n5-c4ccccc4-c4cccc5sc6ccccc6c45)ccc32)cc1 ; c1ccc(-n2c3ccccc3c3cc(-c4ccc5c(c4)c4ccccc4n5-c4cccc5sc6ccccc6c45)ccc32)cc1] 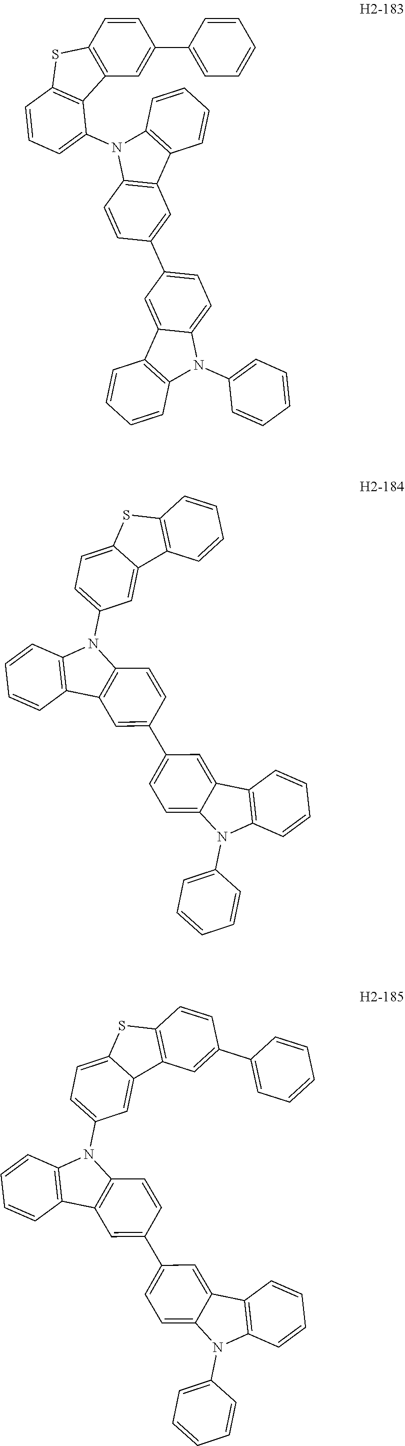 [SMILES: c1ccc(-c2ccc3sc4cccc(-n5c6ccccc6c6cc(-c7ccc8c(c7)c7ccccc7n8-c7ccccc7)ccc65)c4c3c2)cc1 ; c1ccc(-n2c3ccccc3c3cc(-c4ccc5c(c4)c4ccccc4n5-c4ccc5sc6ccccc6c5c4)ccc32)cc1 ; c1ccc(-c2ccc3sc4ccc(-n5c6ccccc6c6cc(-c7ccc8c(c7)c7ccccc7n8-c7ccccc7)ccc65)cc4c3c2)cc1] 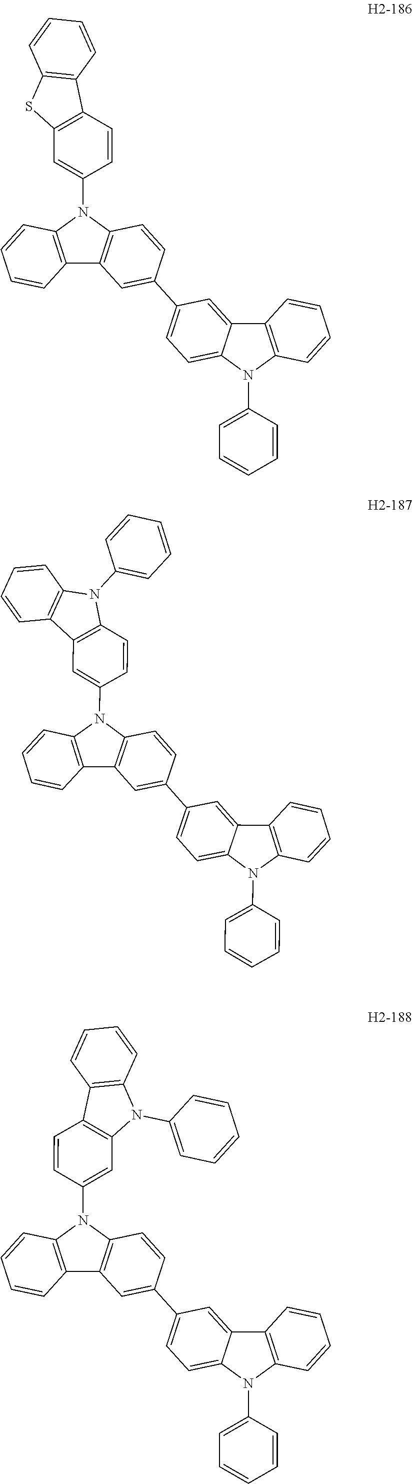 [SMILES: c1ccc(-n2c3ccccc3c3cc(-c4ccc5c(c4)c4ccccc4n5-c4ccc5c(c4)sc4ccccc45)ccc32)cc1 ; c1ccc(-n2c3ccccc3c3cc(-c4ccc5c(c4)c4ccccc4n5-c4ccc5c(c4)c4ccccc4n5-c4ccccc4)ccc32)cc1 ; c1ccc(-n2c3ccccc3c3cc(-c4ccc5c(c4)c4ccccc4n5-c4ccc5c6ccccc6n(-c6ccccc6)c5c4)ccc32)cc1] 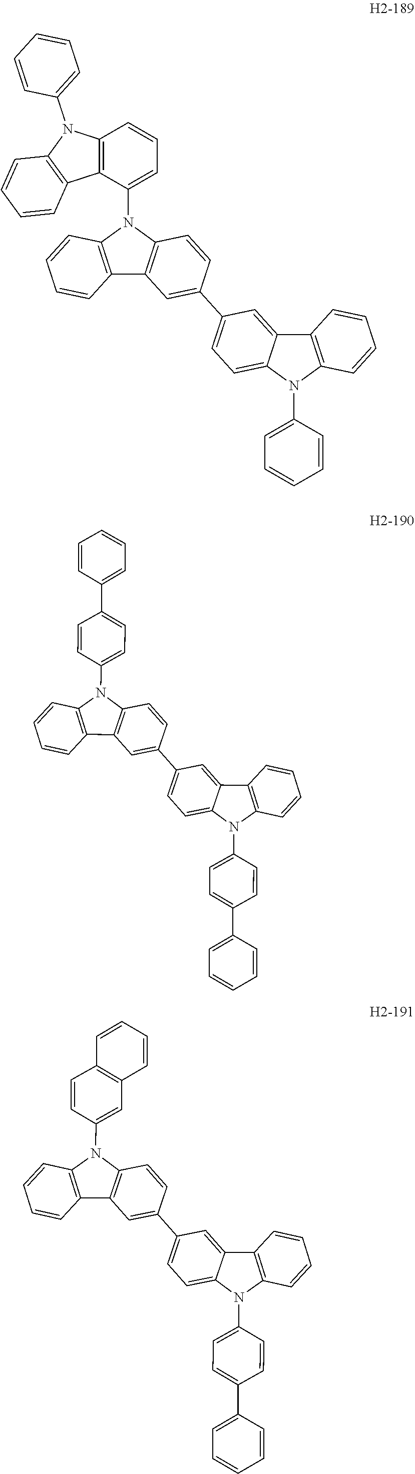 [SMILES: c1ccc(-n2c3ccccc3c3cc(-c4ccc5c(c4)c4ccccc4n5-c4cccc5c4c4ccccc4n5-c4ccccc4)ccc32)cc1 ; c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2ccc(-n3c4ccccc4c4cc(-c5ccc6c(c5)c5ccccc5n6-c5ccc6ccccc6c5)ccc43)cc2)cc1] 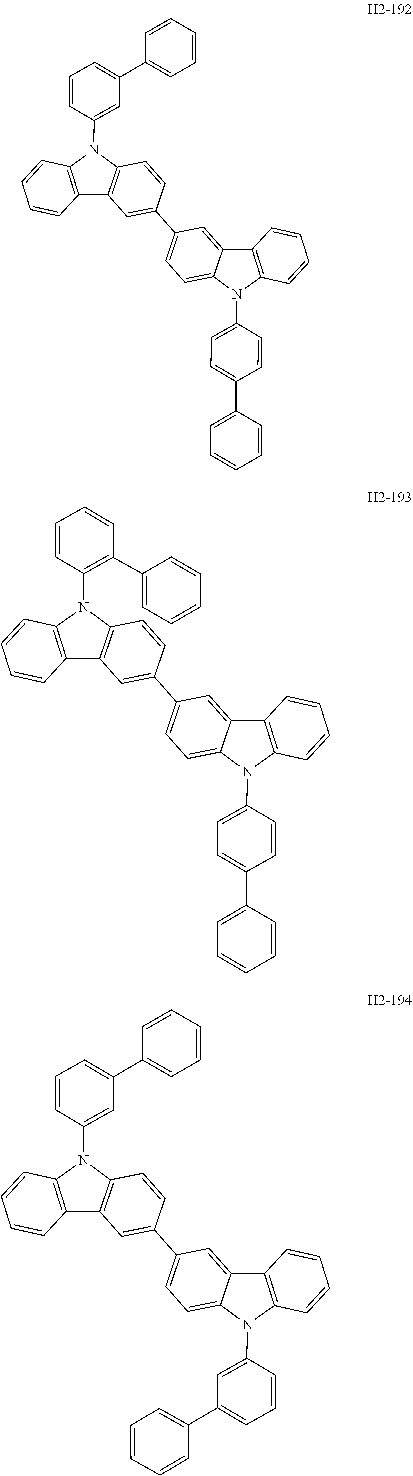 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5-c5ccccc5)ccc43)cc2)cc1 ; c1ccc(-c2cccc(-n3c4ccccc4c4cc(-c5ccc6c(c5)c5ccccc5n6-c5cccc(-c6ccccc6)c5)ccc43)c2)cc1] 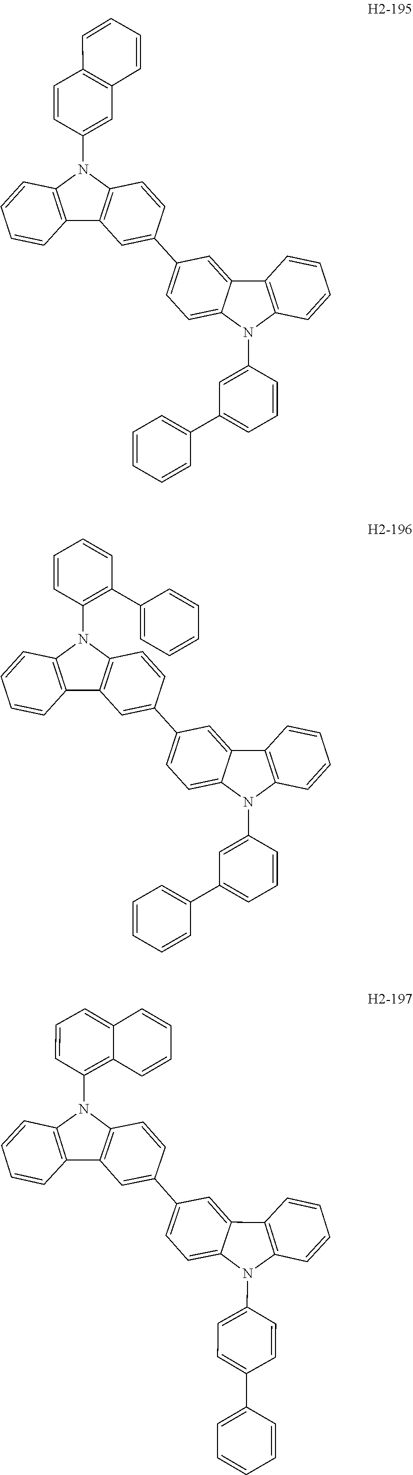 [SMILES: c1ccc(-c2cccc(-n3c4ccccc4c4cc(-c5ccc6c(c5)c5ccccc5n6-c5ccc6ccccc6c5)ccc43)c2)cc1 ; c1ccc(-c2cccc(-n3c4ccccc4c4cc(-c5ccc6c(c5)c5ccccc5n6-c5ccccc5-c5ccccc5)ccc43)c2)cc1 ; c1ccc(-c2ccc(-n3c4ccccc4c4cc(-c5ccc6c(c5)c5ccccc5n6-c5cccc6ccccc56)ccc43)cc2)cc1] 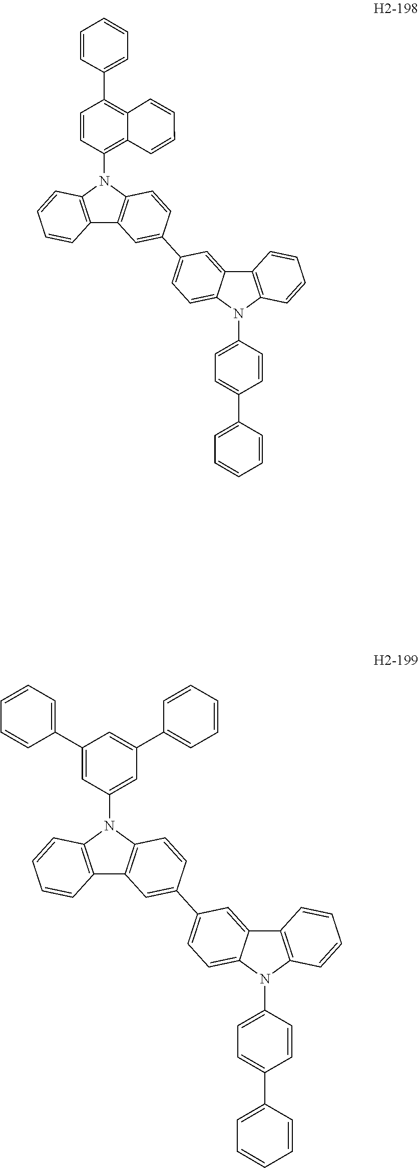 [SMILES: c1ccc(-c2ccc(-n3c4ccccc4c4cc(-c5ccc6c(c5)c5ccccc5n6-c5ccc(-c6ccccc6)c6ccccc56)ccc43)cc2)cc1 ; c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1] 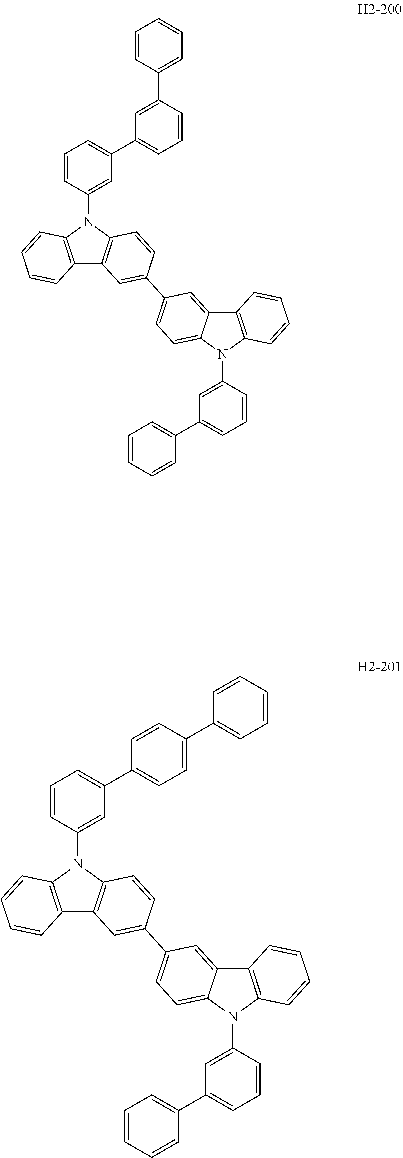 [SMILES: c1ccc(-c2cccc(-c3cccc(-n4c5ccccc5c5cc(-c6ccc7c(c6)c6ccccc6n7-c6cccc(-c7ccccc7)c6)ccc54)c3)c2)cc1 ; c1ccc(-c2ccc(-c3cccc(-n4c5ccccc5c5cc(-c6ccc7c(c6)c6ccccc6n7-c6cccc(-c7ccccc7)c6)ccc54)c3)cc2)cc1] 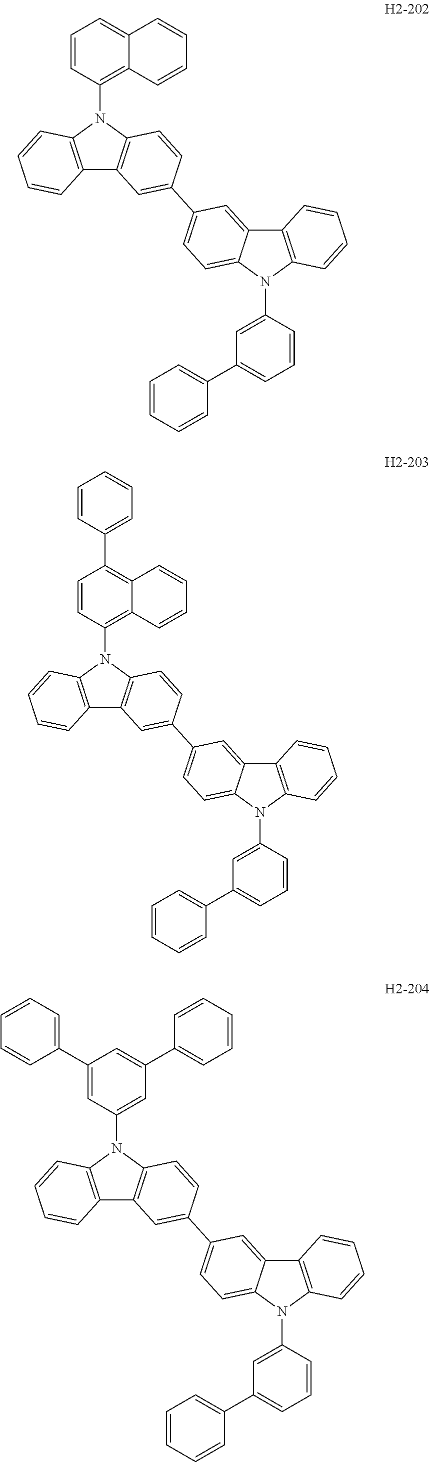 [SMILES: c1ccc(-c2cccc(-n3c4ccccc4c4cc(-c5ccc6c(c5)c5ccccc5n6-c5cccc6ccccc56)ccc43)c2)cc1 ; c1ccc(-c2cccc(-n3c4ccccc4c4cc(-c5ccc6c(c5)c5ccccc5n6-c5ccc(-c6ccccc6)c6ccccc56)ccc43)c2)cc1 ; c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1] 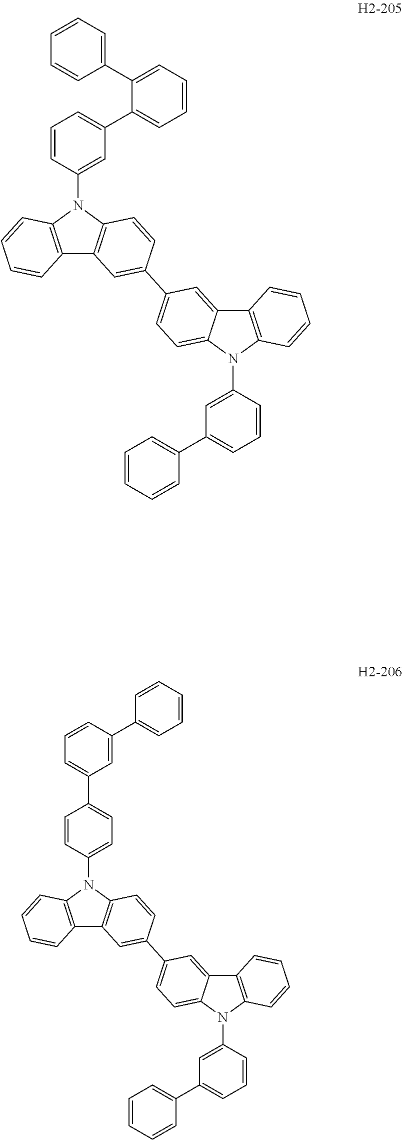 [SMILES: c1ccc(-c2cccc(-n3c4ccccc4c4cc(-c5ccc6c(c5)c5ccccc5n6-c5cccc(-c6ccccc6-c6ccccc6)c5)ccc43)c2)cc1 ; c1ccc(-c2cccc(-c3ccc(-n4c5ccccc5c5cc(-c6ccc7c(c6)c6ccccc6n7-c6cccc(-c7ccccc7)c6)ccc54)cc3)c2)cc1] 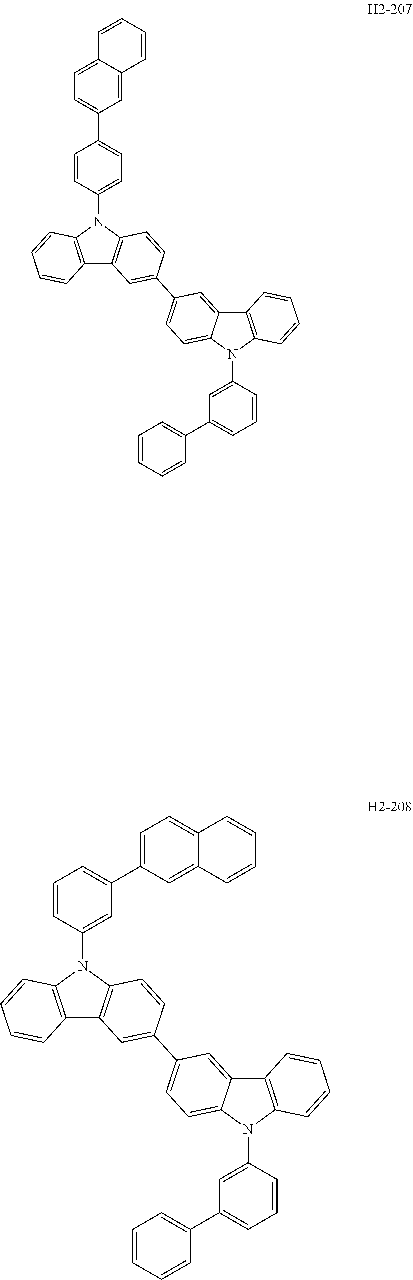 [SMILES: c1ccc(-c2cccc(-n3c4ccccc4c4cc(-c5ccc6c(c5)c5ccccc5n6-c5ccc(-c6ccc7ccccc7c6)cc5)ccc43)c2)cc1 ; c1ccc(-c2cccc(-n3c4ccccc4c4cc(-c5ccc6c(c5)c5ccccc5n6-c5cccc(-c6ccc7ccccc7c6)c5)ccc43)c2)cc1] 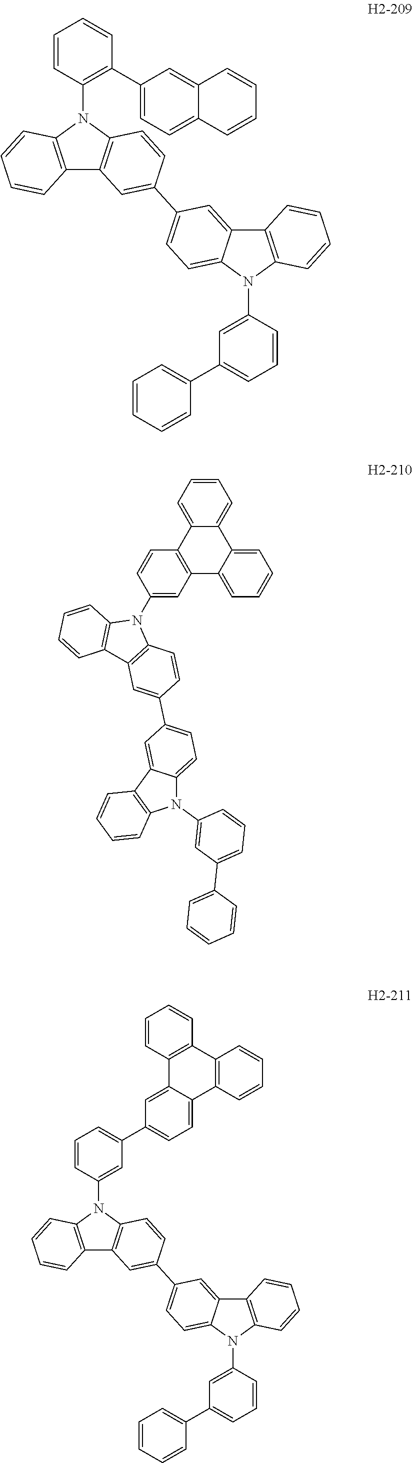 [SMILES: c1ccc(-c2cccc(-n3c4ccccc4c4cc(-c5ccc6c(c5)c5ccccc5n6-c5ccccc5-c5ccc6ccccc6c5)ccc43)c2)cc1 ; c1ccc(-c2cccc(-n3c4ccccc4c4cc(-c5ccc6c(c5)c5ccccc5n6-c5ccc6c7ccccc7c7ccccc7c6c5)ccc43)c2)cc1 ; c1ccc(-c2cccc(-n3c4ccccc4c4cc(-c5ccc6c(c5)c5ccccc5n6-c5cccc(-c6ccc7c8ccccc8c8ccccc8c7c6)c5)ccc43)c2)cc1] 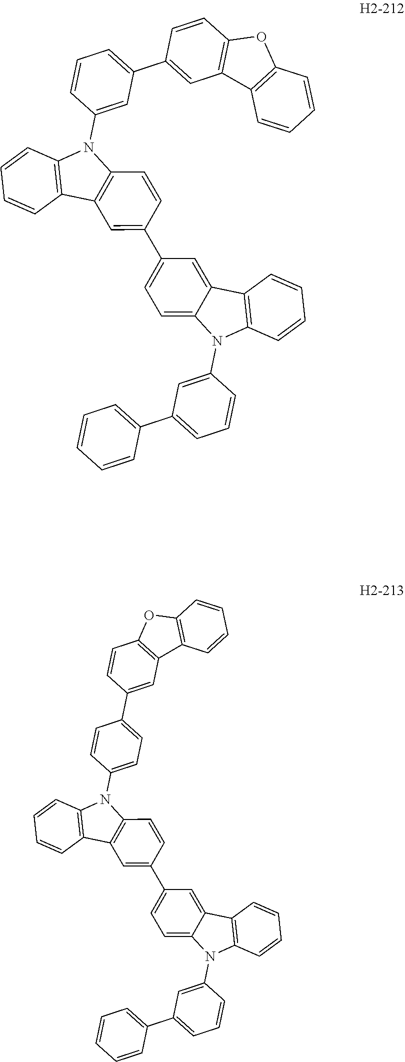 [SMILES: c1ccc(-c2cccc(-n3c4ccccc4c4cc(-c5ccc6c(c5)c5ccccc5n6-c5cccc(-c6ccc7oc8ccccc8c7c6)c5)ccc43)c2)cc1 ; c1ccc(-c2cccc(-n3c4ccccc4c4cc(-c5ccc6c(c5)c5ccccc5n6-c5ccc(-c6ccc7oc8ccccc8c7c6)cc5)ccc43)c2)cc1] 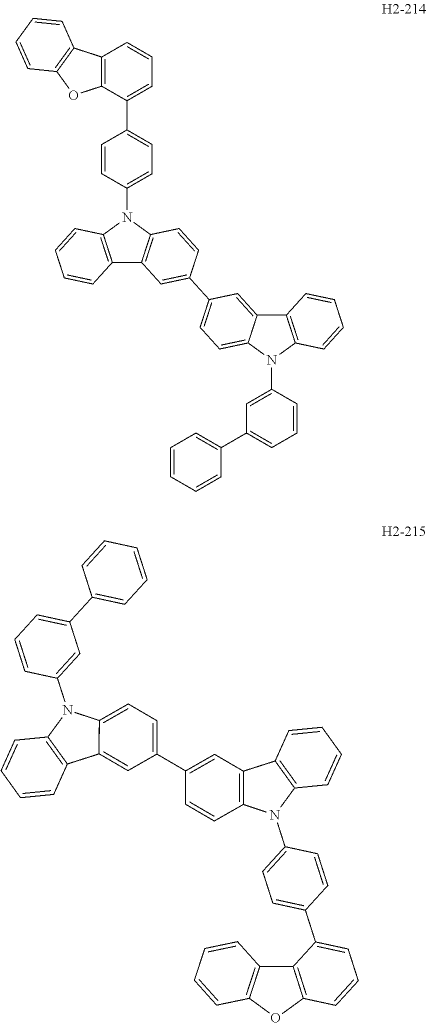 [SMILES: c1ccc(-c2cccc(-n3c4ccccc4c4cc(-c5ccc6c(c5)c5ccccc5n6-c5ccc(-c6cccc7c6oc6ccccc67)cc5)ccc43)c2)cc1 ; c1ccc(-c2cccc(-n3c4ccccc4c4cc(-c5ccc6c(c5)c5ccccc5n6-c5ccc(-c6cccc7oc8ccccc8c67)cc5)ccc43)c2)cc1] 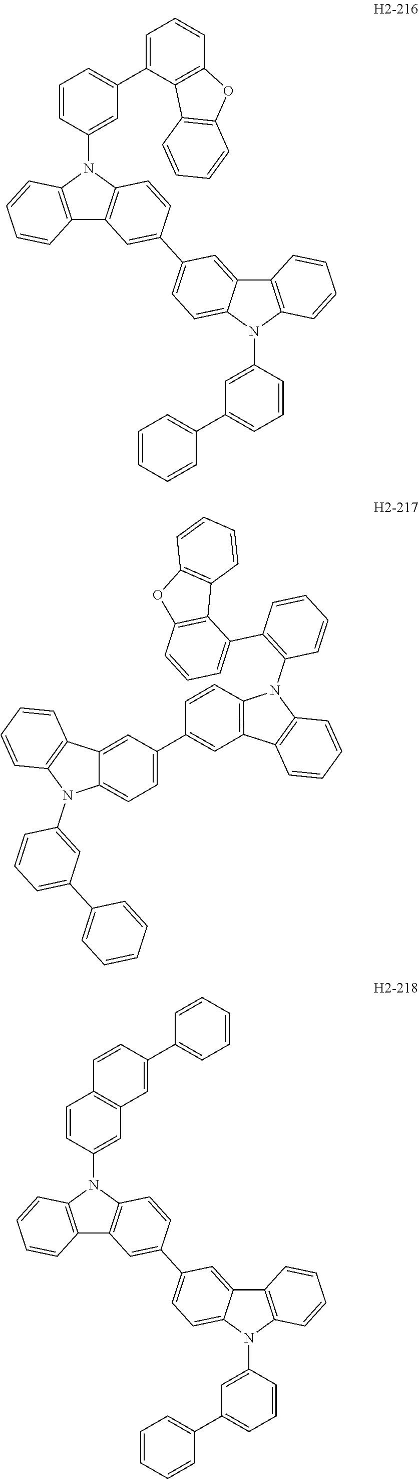 [SMILES: c1ccc(-c2cccc(-n3c4ccccc4c4cc(-c5ccc6c(c5)c5ccccc5n6-c5cccc(-c6cccc7oc8ccccc8c67)c5)ccc43)c2)cc1 ; c1ccc(-c2cccc(-n3c4ccccc4c4cc(-c5ccc6c(c5)c5ccccc5n6-c5ccccc5-c5cccc6oc7ccccc7c56)ccc43)c2)cc1 ; c1ccc(-c2cccc(-n3c4ccccc4c4cc(-c5ccc6c(c5)c5ccccc5n6-c5ccc6ccc(-c7ccccc7)cc6c5)ccc43)c2)cc1] 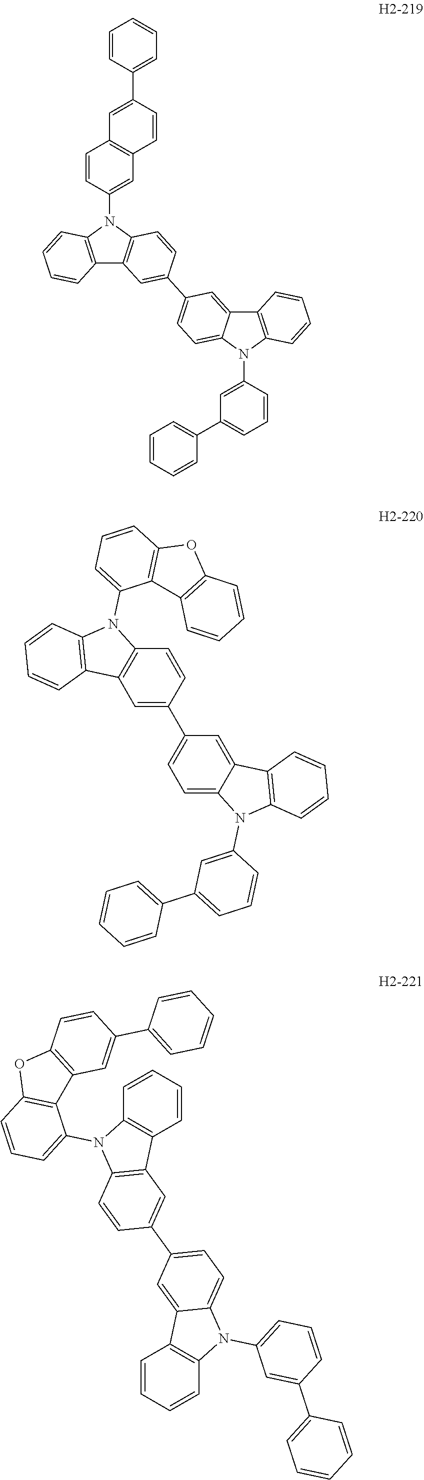 [SMILES: c1ccc(-c2cccc(-n3c4ccccc4c4cc(-c5ccc6c(c5)c5ccccc5n6-c5ccc6cc(-c7ccccc7)ccc6c5)ccc43)c2)cc1 ; c1ccc(-c2cccc(-n3c4ccccc4c4cc(-c5ccc6c(c5)c5ccccc5n6-c5cccc6oc7ccccc7c56)ccc43)c2)cc1 ; c1ccc(-c2cccc(-n3c4ccccc4c4cc(-c5ccc6c(c5)c5ccccc5n6-c5cccc6oc7ccc(-c8ccccc8)cc7c56)ccc43)c2)cc1] 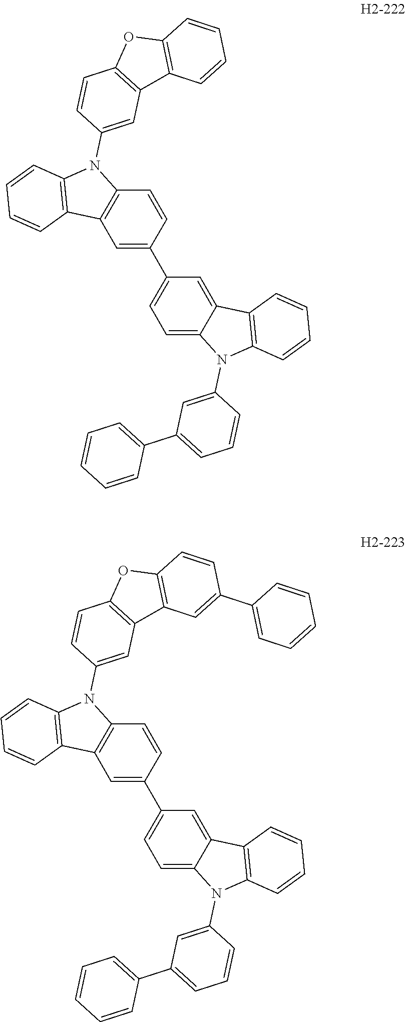 [SMILES: c1ccc(-c2cccc(-n3c4ccccc4c4cc(-c5ccc6c(c5)c5ccccc5n6-c5ccc6oc7ccccc7c6c5)ccc43)c2)cc1 ; c1ccc(-c2cccc(-n3c4ccccc4c4cc(-c5ccc6c(c5)c5ccccc5n6-c5ccc6oc7ccc(-c8ccccc8)cc7c6c5)ccc43)c2)cc1] 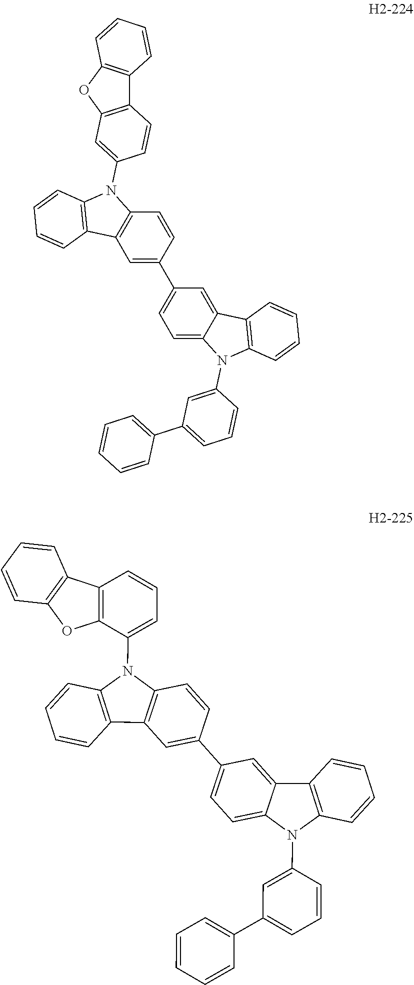 [SMILES: c1ccc(-c2cccc(-n3c4ccccc4c4cc(-c5ccc6c(c5)c5ccccc5n6-c5ccc6c(c5)oc5ccccc56)ccc43)c2)cc1 ; c1ccc(-c2cccc(-n3c4ccccc4c4cc(-c5ccc6c(c5)c5ccccc5n6-c5cccc6c5oc5ccccc56)ccc43)c2)cc1] 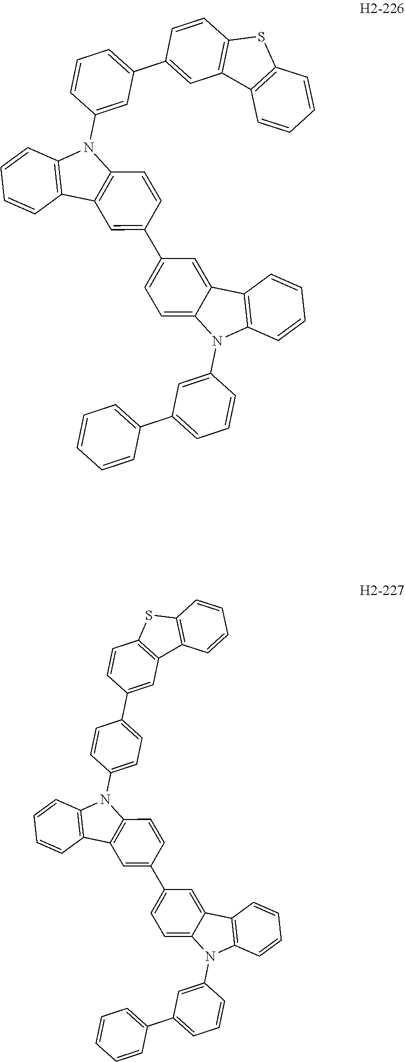 [SMILES: c1ccc(-c2cccc(-n3c4ccccc4c4cc(-c5ccc6c(c5)c5ccccc5n6-c5cccc(-c6ccc7sc8ccccc8c7c6)c5)ccc43)c2)cc1 ; c1ccc(-c2cccc(-n3c4ccccc4c4cc(-c5ccc6c(c5)c5ccccc5n6-c5ccc(-c6ccc7sc8ccccc8c7c6)cc5)ccc43)c2)cc1] 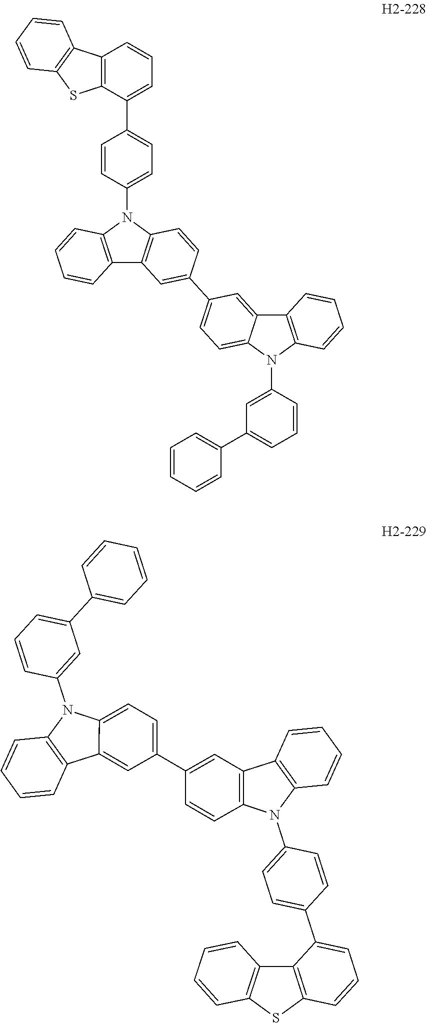 [SMILES: c1ccc(-c2cccc(-n3c4ccccc4c4cc(-c5ccc6c(c5)c5ccccc5n6-c5ccc(-c6cccc7c6sc6ccccc67)cc5)ccc43)c2)cc1 ; c1ccc(-c2cccc(-n3c4ccccc4c4cc(-c5ccc6c(c5)c5ccccc5n6-c5ccc(-c6cccc7sc8ccccc8c67)cc5)ccc43)c2)cc1] 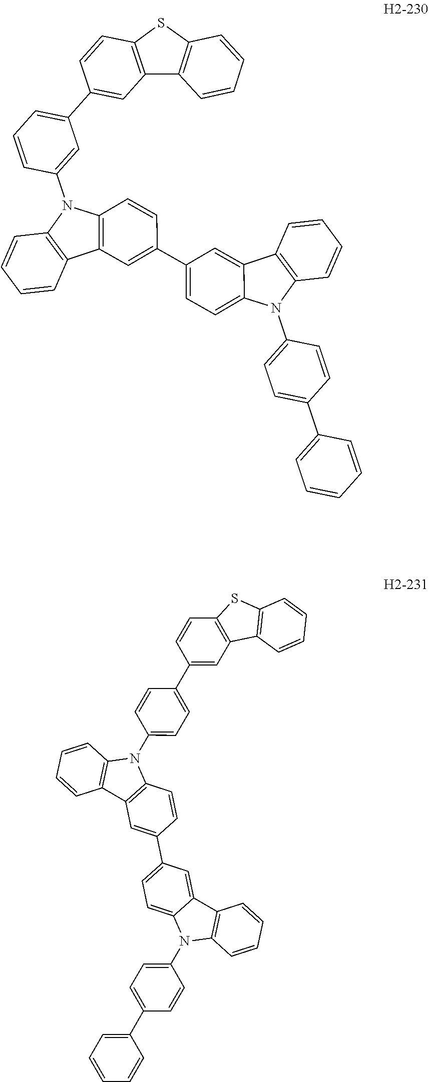 [SMILES: c1ccc(-c2ccc(-n3c4ccccc4c4cc(-c5ccc6c(c5)c5ccccc5n6-c5cccc(-c6ccc7sc8ccccc8c7c6)c5)ccc43)cc2)cc1 ; c1ccc(-c2ccc(-n3c4ccccc4c4cc(-c5ccc6c(c5)c5ccccc5n6-c5ccc(-c6ccc7sc8ccccc8c7c6)cc5)ccc43)cc2)cc1] 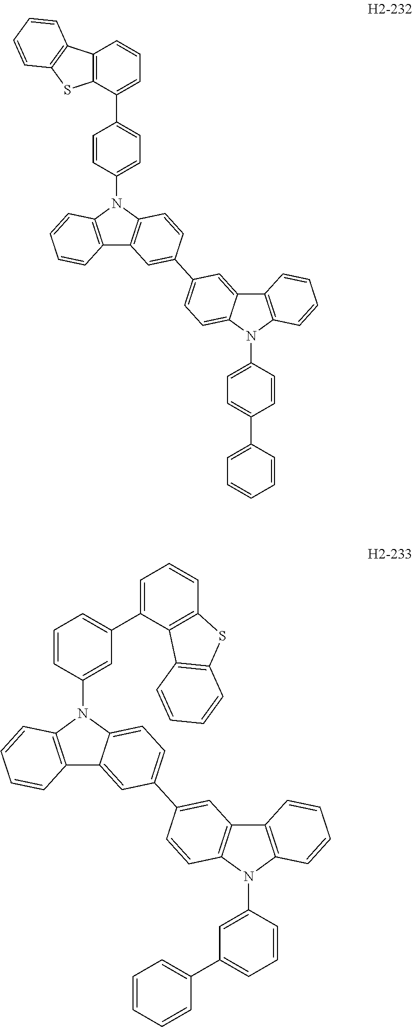 [SMILES: c1ccc(-c2ccc(-n3c4ccccc4c4cc(-c5ccc6c(c5)c5ccccc5n6-c5ccc(-c6cccc7c6sc6ccccc67)cc5)ccc43)cc2)cc1 ; c1ccc(-c2cccc(-n3c4ccccc4c4cc(-c5ccc6c(c5)c5ccccc5n6-c5cccc(-c6cccc7sc8ccccc8c67)c5)ccc43)c2)cc1] 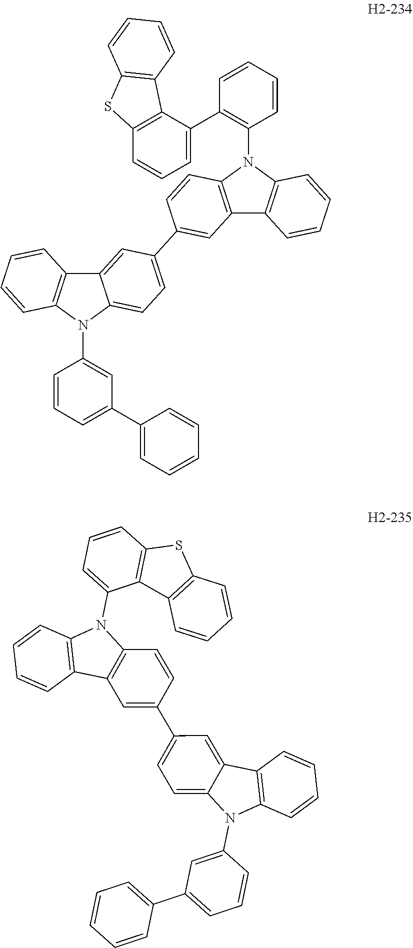 [SMILES: c1ccc(-c2cccc(-n3c4ccccc4c4cc(-c5ccc6c(c5)c5ccccc5n6-c5ccccc5-c5cccc6sc7ccccc7c56)ccc43)c2)cc1 ; c1ccc(-c2cccc(-n3c4ccccc4c4cc(-c5ccc6c(c5)c5ccccc5n6-c5cccc6sc7ccccc7c56)ccc43)c2)cc1] 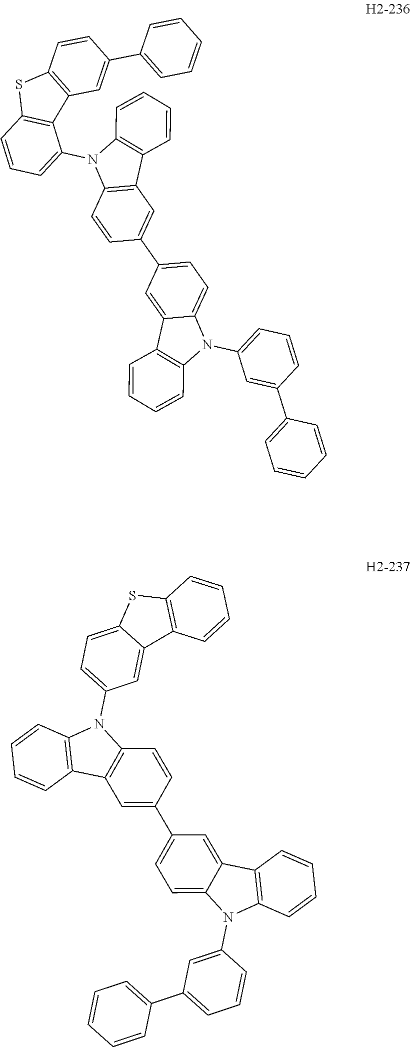 [SMILES: c1ccc(-c2cccc(-n3c4ccccc4c4cc(-c5ccc6c(c5)c5ccccc5n6-c5cccc6sc7ccc(-c8ccccc8)cc7c56)ccc43)c2)cc1 ; c1ccc(-c2cccc(-n3c4ccccc4c4cc(-c5ccc6c(c5)c5ccccc5n6-c5ccc6sc7ccccc7c6c5)ccc43)c2)cc1] 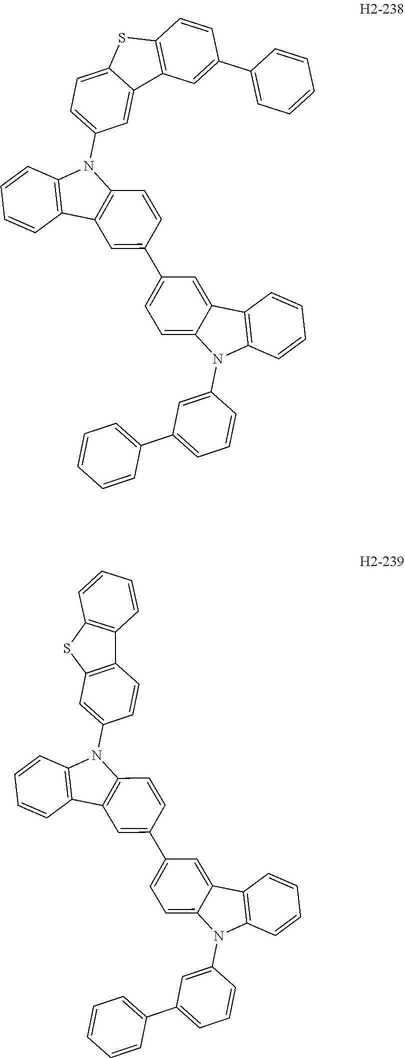 [SMILES: c1ccc(-c2cccc(-n3c4ccccc4c4cc(-c5ccc6c(c5)c5ccccc5n6-c5ccc6sc7ccc(-c8ccccc8)cc7c6c5)ccc43)c2)cc1 ; c1ccc(-c2cccc(-n3c4ccccc4c4cc(-c5ccc6c(c5)c5ccccc5n6-c5ccc6c(c5)sc5ccccc56)ccc43)c2)cc1] 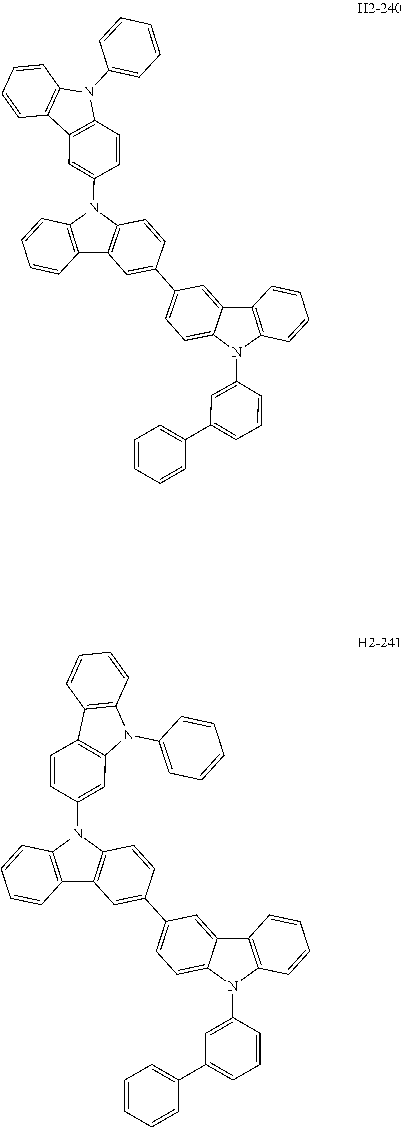 [SMILES: c1ccc(-c2cccc(-n3c4ccccc4c4cc(-c5ccc6c(c5)c5ccccc5n6-c5ccc6c(c5)c5ccccc5n6-c5ccccc5)ccc43)c2)cc1 ; c1ccc(-c2cccc(-n3c4ccccc4c4cc(-c5ccc6c(c5)c5ccccc5n6-c5ccc6c7ccccc7n(-c7ccccc7)c6c5)ccc43)c2)cc1] 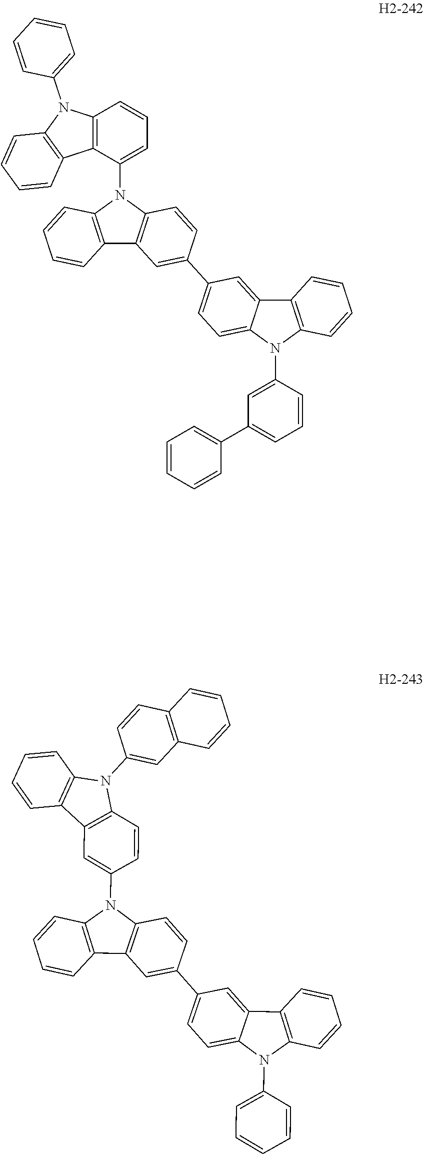 [SMILES: c1ccc(-c2cccc(-n3c4ccccc4c4cc(-c5ccc6c(c5)c5ccccc5n6-c5cccc6c5c5ccccc5n6-c5ccccc5)ccc43)c2)cc1 ; c1ccc(-n2c3ccccc3c3cc(-c4ccc5c(c4)c4ccccc4n5-c4ccc5c(c4)c4ccccc4n5-c4ccc5ccccc5c4)ccc32)cc1] 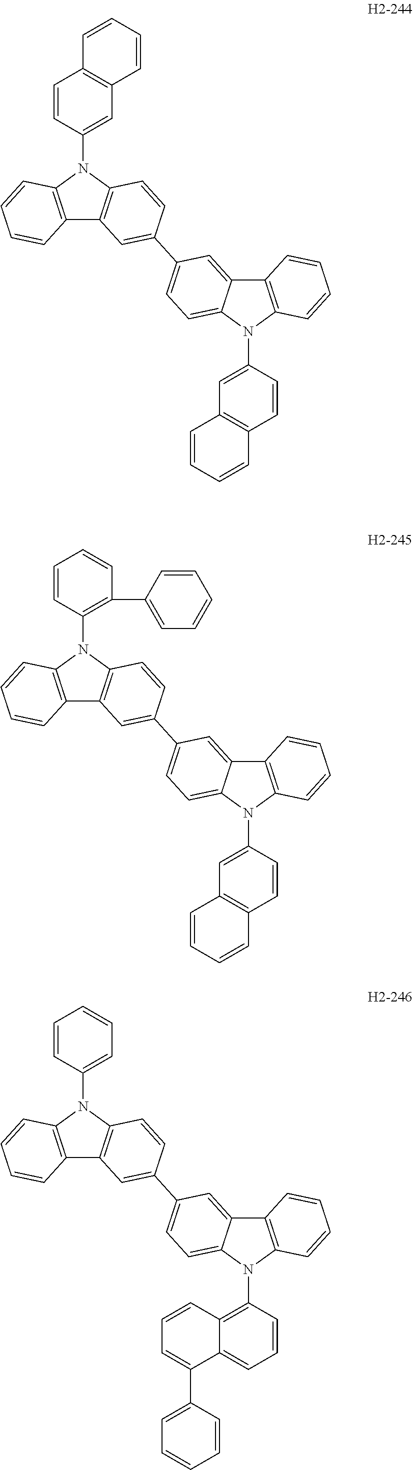 [SMILES: c1ccc2cc(-n3c4ccccc4c4cc(-c5ccc6c(c5)c5ccccc5n6-c5ccc6ccccc6c5)ccc43)ccc2c1 ; c1ccc(-c2ccccc2-n2c3ccccc3c3cc(-c4ccc5c(c4)c4ccccc4n5-c4ccc5ccccc5c4)ccc32)cc1 ; c1ccc(-c2cccc3c(-n4c5ccccc5c5cc(-c6ccc7c(c6)c6ccccc6n7-c6ccccc6)ccc54)cccc23)cc1] 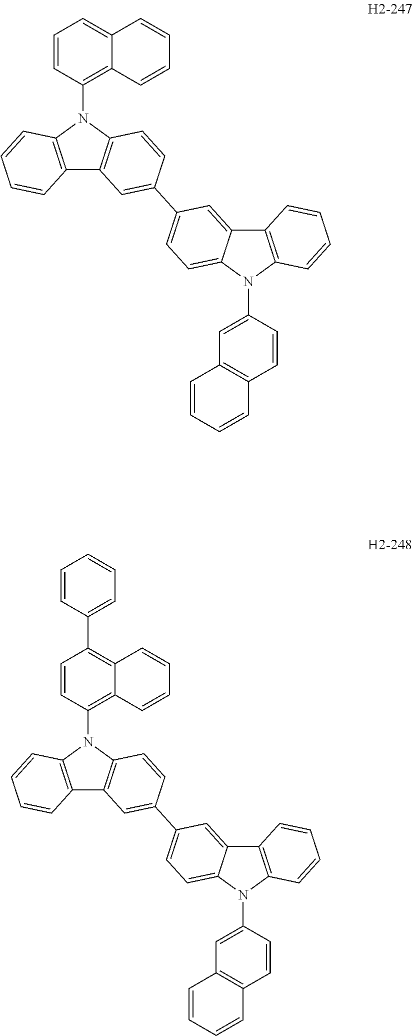 [SMILES: c1ccc2cc(-n3c4ccccc4c4cc(-c5ccc6c(c5)c5ccccc5n6-c5cccc6ccccc56)ccc43)ccc2c1 ; c1ccc(-c2ccc(-n3c4ccccc4c4cc(-c5ccc6c(c5)c5ccccc5n6-c5ccc6ccccc6c5)ccc43)c3ccccc23)cc1] 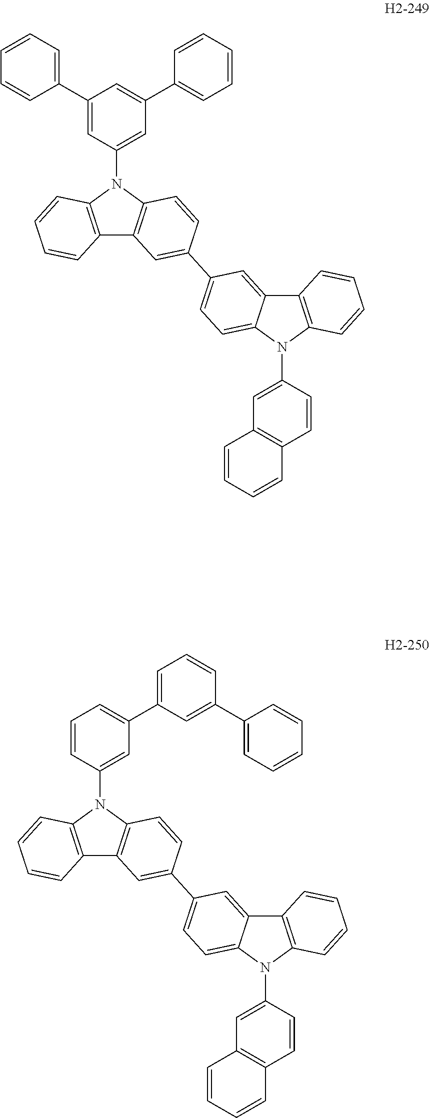 [SMILES: c1ccc(-c2cc(-c3ccccc3)cc(-n3c4ccccc4c4cc(-c5ccc6c(c5)c5ccccc5n6-c5ccc6ccccc6c5)ccc43)c2)cc1 ; c1ccc(-c2cccc(-c3cccc(-n4c5ccccc5c5cc(-c6ccc7c(c6)c6ccccc6n7-c6ccc7ccccc7c6)ccc54)c3)c2)cc1] 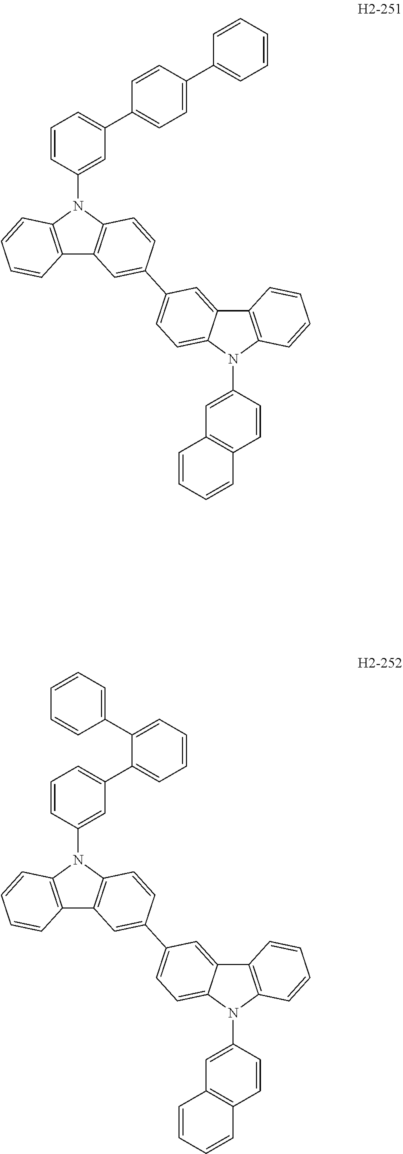 [SMILES: c1ccc(-c2ccc(-c3cccc(-n4c5ccccc5c5cc(-c6ccc7c(c6)c6ccccc6n7-c6ccc7ccccc7c6)ccc54)c3)cc2)cc1 ; c1ccc(-c2ccccc2-c2cccc(-n3c4ccccc4c4cc(-c5ccc6c(c5)c5ccccc5n6-c5ccc6ccccc6c5)ccc43)c2)cc1] 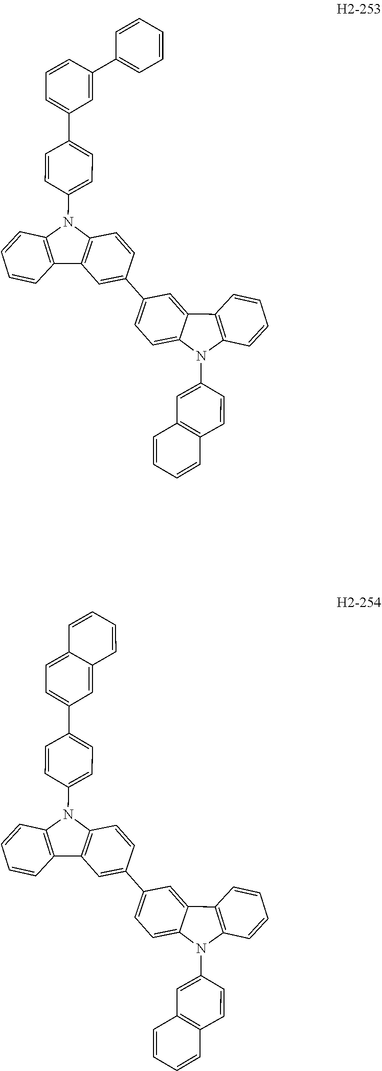 [SMILES: c1ccc(-c2cccc(-c3ccc(-n4c5ccccc5c5cc(-c6ccc7c(c6)c6ccccc6n7-c6ccc7ccccc7c6)ccc54)cc3)c2)cc1 ; c1ccc2cc(-c3ccc(-n4c5ccccc5c5cc(-c6ccc7c(c6)c6ccccc6n7-c6ccc7ccccc7c6)ccc54)cc3)ccc2c1] 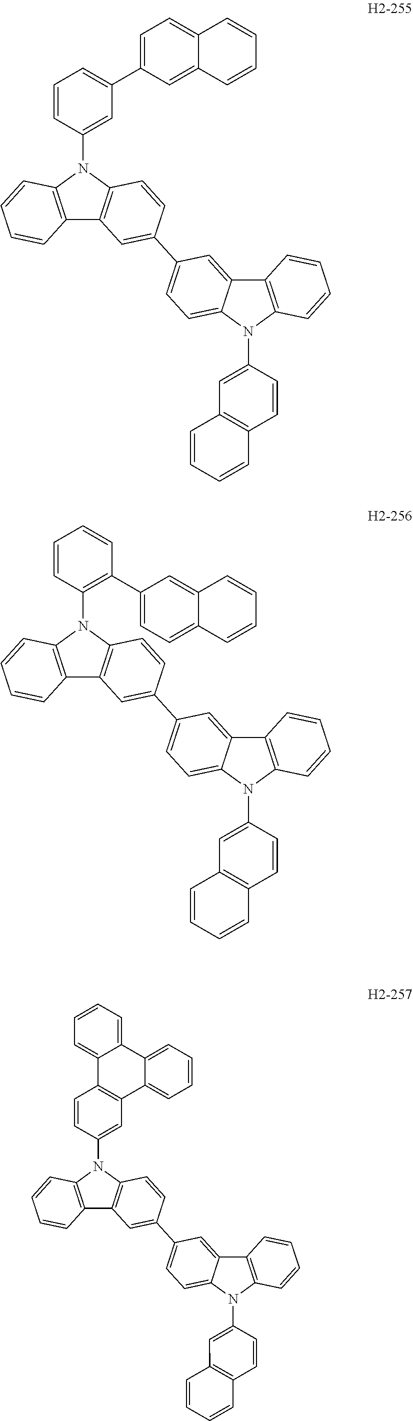 [SMILES: c1cc(-c2ccc3ccccc3c2)cc(-n2c3ccccc3c3cc(-c4ccc5c(c4)c4ccccc4n5-c4ccc5ccccc5c4)ccc32)c1 ; c1ccc(-n2c3ccccc3c3cc(-c4ccc5c(c4)c4ccccc4n5-c4ccc5ccccc5c4)ccc32)c(-c2ccc3ccccc3c2)c1 ; c1ccc2cc(-n3c4ccccc4c4cc(-c5ccc6c(c5)c5ccccc5n6-c5ccc6c7ccccc7c7ccccc7c6c5)ccc43)ccc2c1] 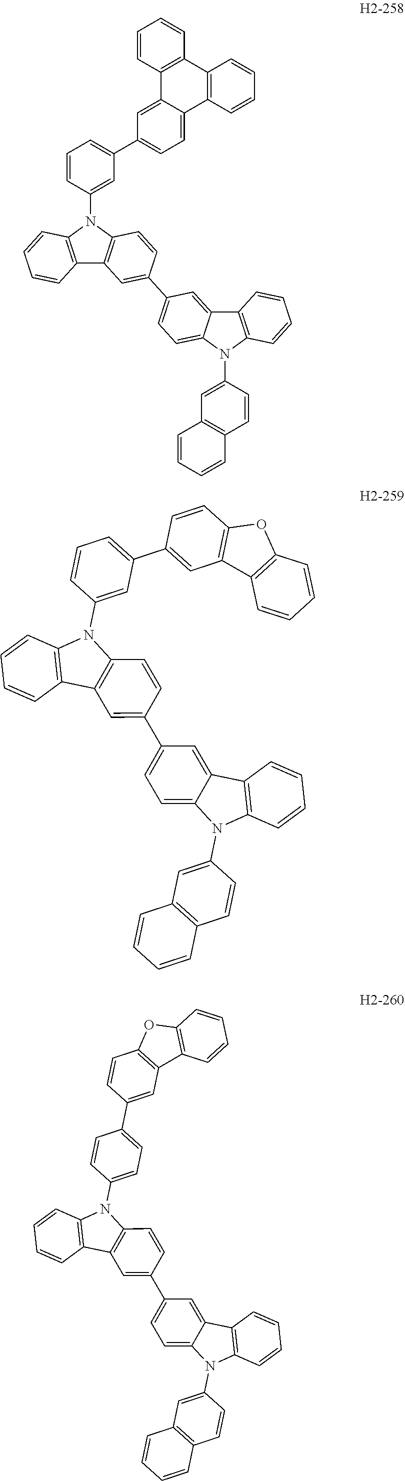 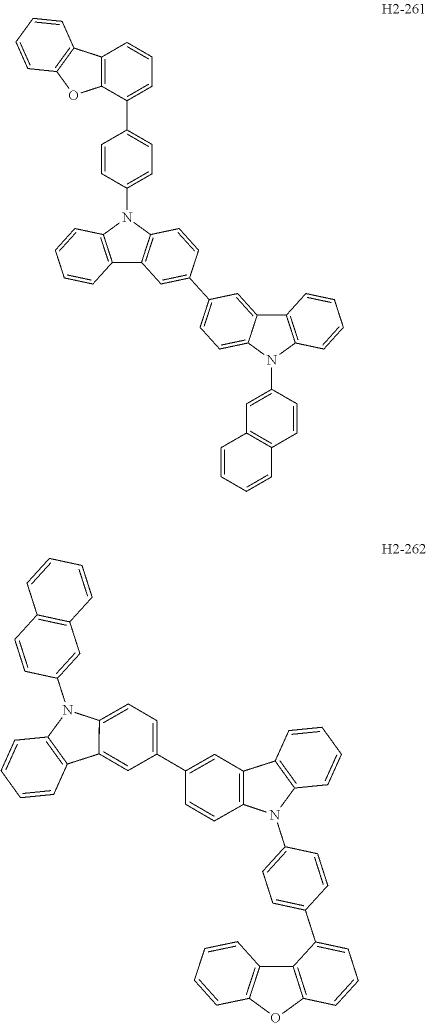 [SMILES: c1ccc2cc(-n3c4ccccc4c4cc(-c5ccc6c(c5)c5ccccc5n6-c5ccc(-c6cccc7c6oc6ccccc67)cc5)ccc43)ccc2c1 ; c1ccc2cc(-n3c4ccccc4c4cc(-c5ccc6c(c5)c5ccccc5n6-c5ccc(-c6cccc7oc8ccccc8c67)cc5)ccc43)ccc2c1] 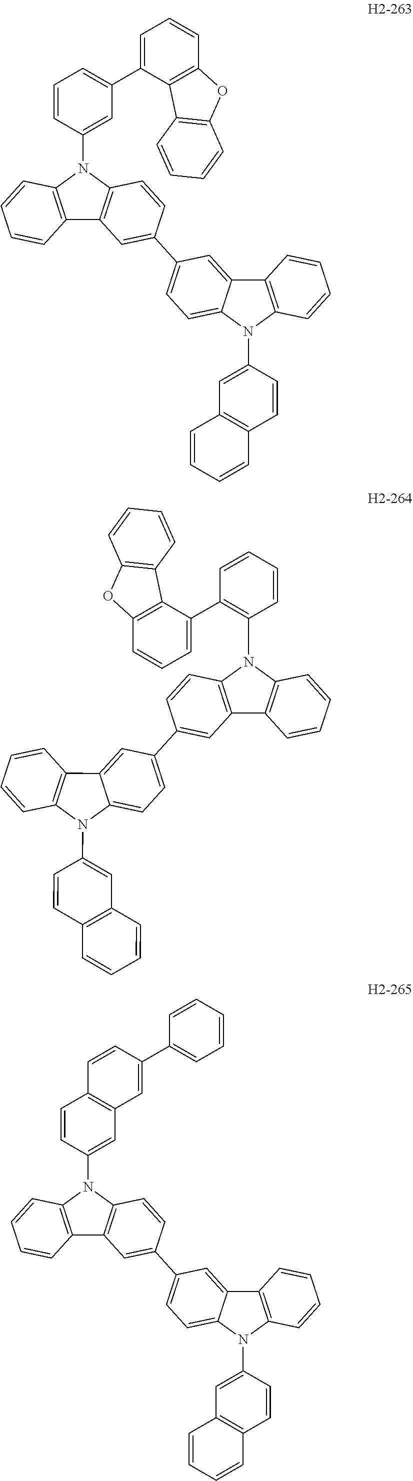 [SMILES: c1cc(-c2cccc3oc4ccccc4c23)cc(-n2c3ccccc3c3cc(-c4ccc5c(c4)c4ccccc4n5-c4ccc5ccccc5c4)ccc32)c1 ; c1ccc(-n2c3ccccc3c3cc(-c4ccc5c(c4)c4ccccc4n5-c4ccc5ccccc5c4)ccc32)c(-c2cccc3oc4ccccc4c23)c1 ; c1ccc(-c2ccc3ccc(-n4c5ccccc5c5cc(-c6ccc7c(c6)c6ccccc6n7-c6ccc7ccccc7c6)ccc54)cc3c2)cc1] 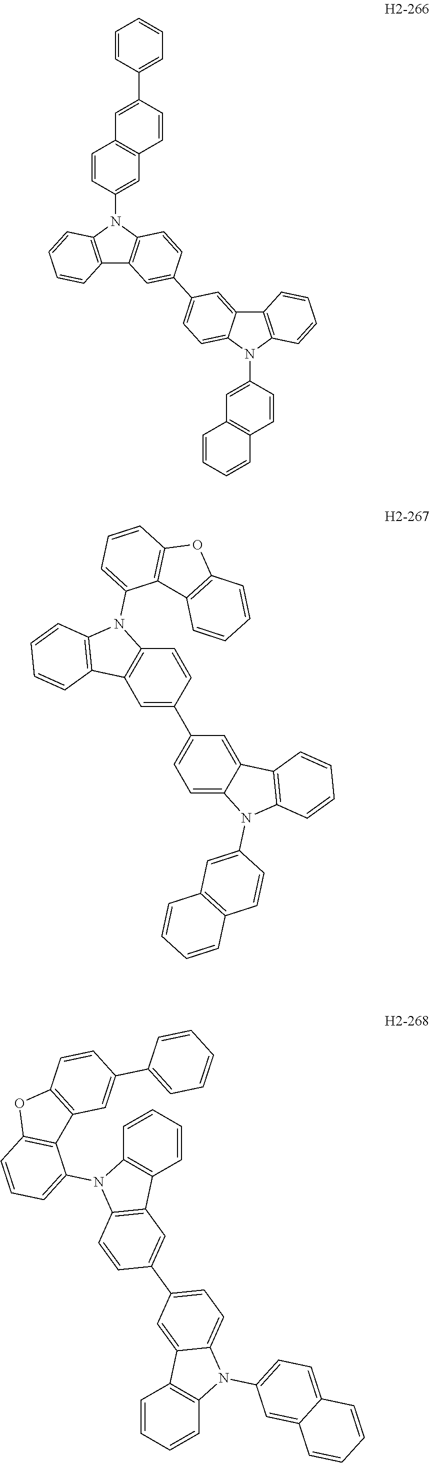 [SMILES: c1ccc(-c2ccc3cc(-n4c5ccccc5c5cc(-c6ccc7c(c6)c6ccccc6n7-c6ccc7ccccc7c6)ccc54)ccc3c2)cc1 ; c1ccc2cc(-n3c4ccccc4c4cc(-c5ccc6c(c5)c5ccccc5n6-c5cccc6oc7ccccc7c56)ccc43)ccc2c1 ; c1ccc(-c2ccc3oc4cccc(-n5c6ccccc6c6cc(-c7ccc8c(c7)c7ccccc7n8-c7ccc8ccccc8c7)ccc65)c4c3c2)cc1] 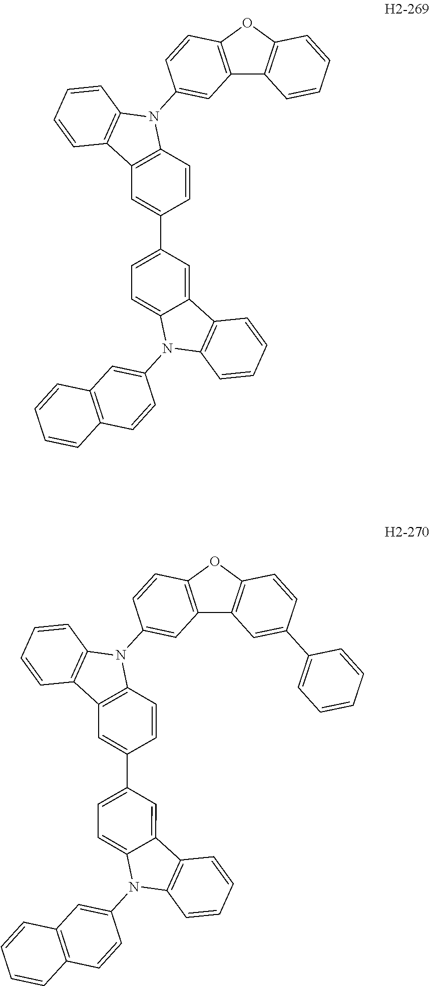 [SMILES: c1ccc2cc(-n3c4ccccc4c4cc(-c5ccc6c(c5)c5ccccc5n6-c5ccc6oc7ccccc7c6c5)ccc43)ccc2c1 ; c1ccc(-c2ccc3oc4ccc(-n5c6ccccc6c6cc(-c7ccc8c(c7)c7ccccc7n8-c7ccc8ccccc8c7)ccc65)cc4c3c2)cc1] 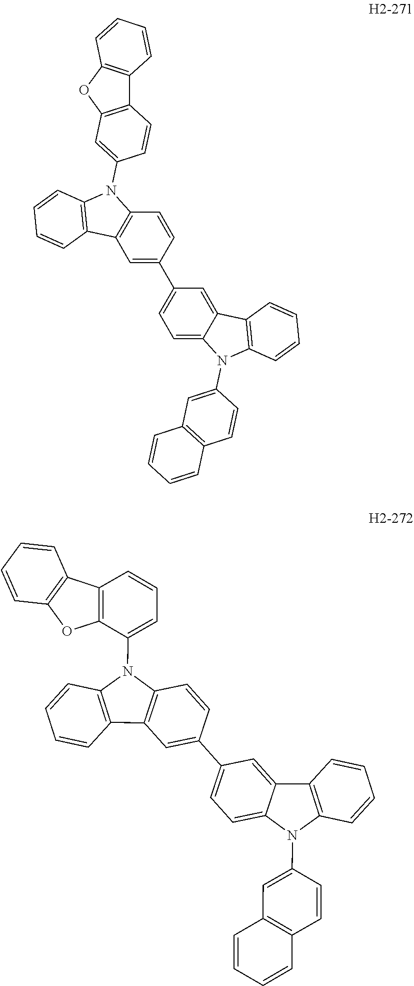 [SMILES: c1ccc2cc(-n3c4ccccc4c4cc(-c5ccc6c(c5)c5ccccc5n6-c5ccc6c(c5)oc5ccccc56)ccc43)ccc2c1 ; c1ccc2cc(-n3c4ccccc4c4cc(-c5ccc6c(c5)c5ccccc5n6-c5cccc6c5oc5ccccc56)ccc43)ccc2c1] 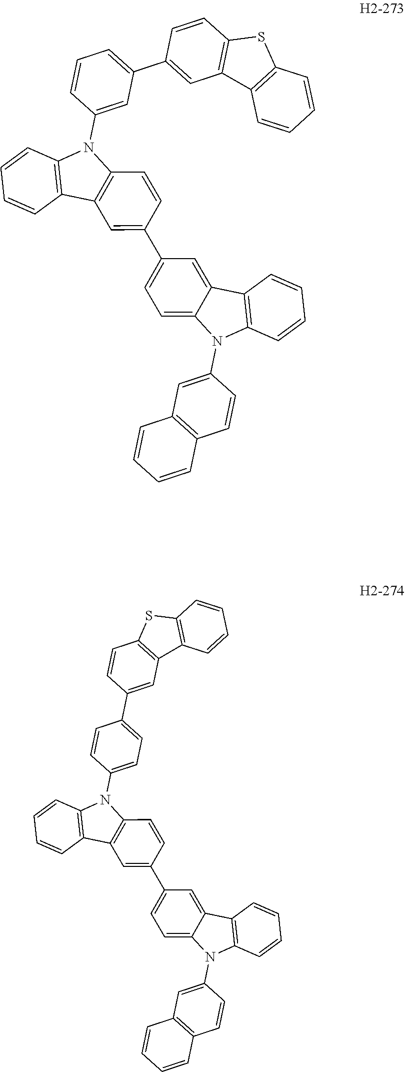 [SMILES: c1cc(-c2ccc3sc4ccccc4c3c2)cc(-n2c3ccccc3c3cc(-c4ccc5c(c4)c4ccccc4n5-c4ccc5ccccc5c4)ccc32)c1 ; c1ccc2cc(-n3c4ccccc4c4cc(-c5ccc6c(c5)c5ccccc5n6-c5ccc(-c6ccc7sc8ccccc8c7c6)cc5)ccc43)ccc2c1] 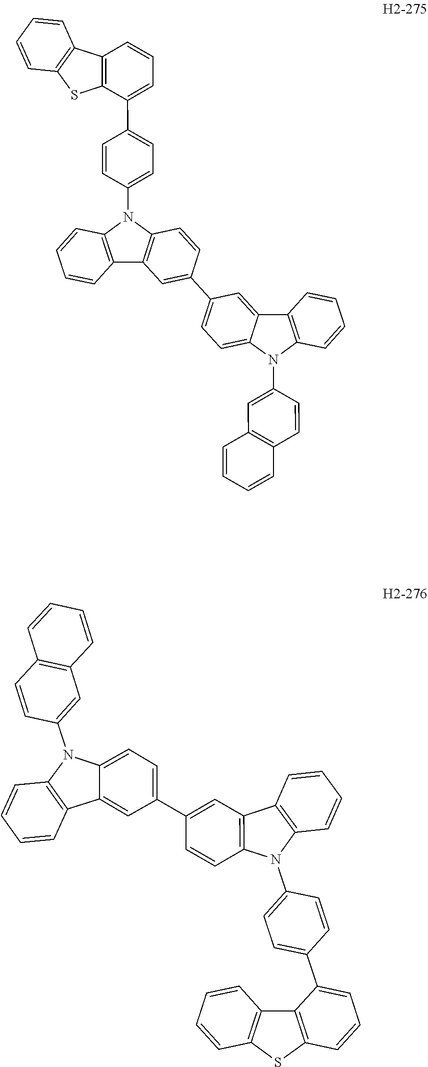 [SMILES: c1ccc2cc(-n3c4ccccc4c4cc(-c5ccc6c(c5)c5ccccc5n6-c5ccc(-c6cccc7c6sc6ccccc67)cc5)ccc43)ccc2c1 ; c1ccc2cc(-n3c4ccccc4c4cc(-c5ccc6c(c5)c5ccccc5n6-c5ccc(-c6cccc7sc8ccccc8c67)cc5)ccc43)ccc2c1] 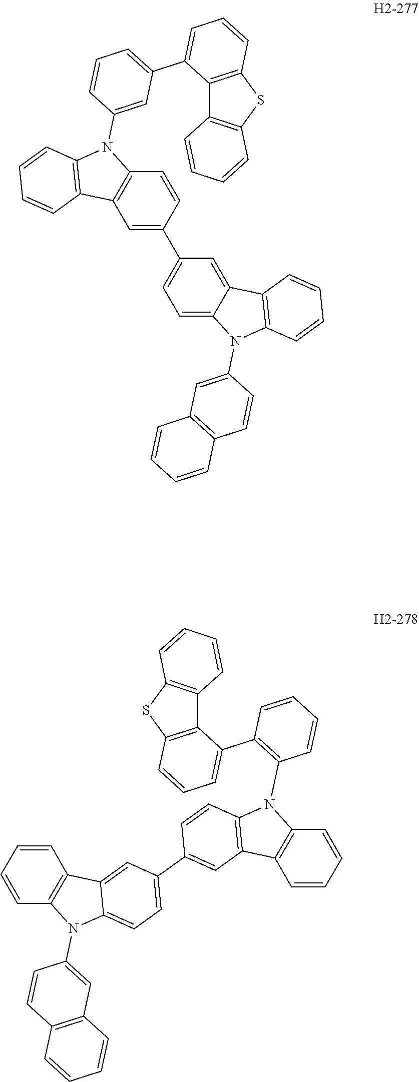 [SMILES: c1cc(-c2cccc3sc4ccccc4c23)cc(-n2c3ccccc3c3cc(-c4ccc5c(c4)c4ccccc4n5-c4ccc5ccccc5c4)ccc32)c1 ; c1ccc(-n2c3ccccc3c3cc(-c4ccc5c(c4)c4ccccc4n5-c4ccc5ccccc5c4)ccc32)c(-c2cccc3sc4ccccc4c23)c1] 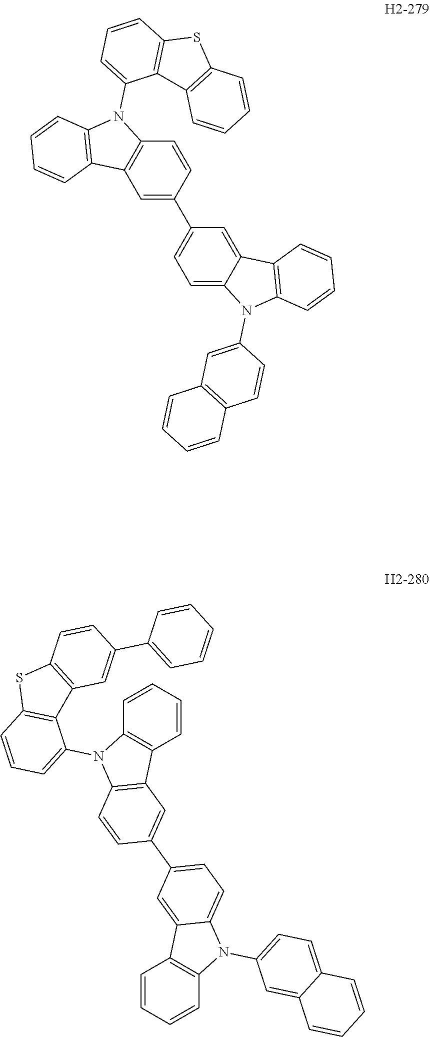 [SMILES: c1ccc2cc(-n3c4ccccc4c4cc(-c5ccc6c(c5)c5ccccc5n6-c5cccc6sc7ccccc7c56)ccc43)ccc2c1 ; c1ccc(-c2ccc3sc4cccc(-n5c6ccccc6c6cc(-c7ccc8c(c7)c7ccccc7n8-c7ccc8ccccc8c7)ccc65)c4c3c2)cc1]
in the compounds, Dₙ means that n number of hydrogens are substituted with deuterium.

11. An organic electroluminescent compound represented by the following formula 3: 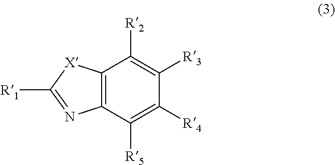
in formula 3,
X represents O or S;
R′₁ represents a phenyl unsubstituted or substituted with deuterium;
R′₂ to R′₅ each independently represent hydrogen, deuterium, a substituted or unsubstituted (C6-C30)aryl, or the following formula a′; with a proviso that at least one of R′₂ to R′₅ represents formula a′; 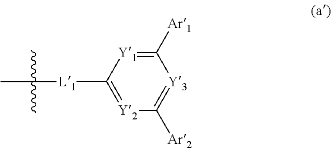
in formula a′,
L′₁ represents a single bond, or a substituted or unsubstituted (C6-C30)arylene;
Y′₁ to Y′₃ each independently represent N or CH; with a proviso that at least two of Y₁ to Y′₃ represent N;
Ar′₁ and Ar′₂ each independently represent a substituted or unsubstituted (C6-C30)aryl, a substituted or unsubstituted carbazolyl, a substituted or unsubstituted dibenzofuranyl, or a substituted or unsubstituted dibenzothiophenyl; with a proviso that at least one of Ar′₁ and Ar′₂ is a substituted or unsubstituted carbazolyl, a substituted or unsubstituted dibenzofuranyl, or a substituted or unsubstituted dibenzothiophenyl; and
Ar′₁ and Ar′₂ are different from each other.

12. The organic electroluminescent compound according to claim 11, wherein the compound represented by formula 3 is selected from the following compounds:

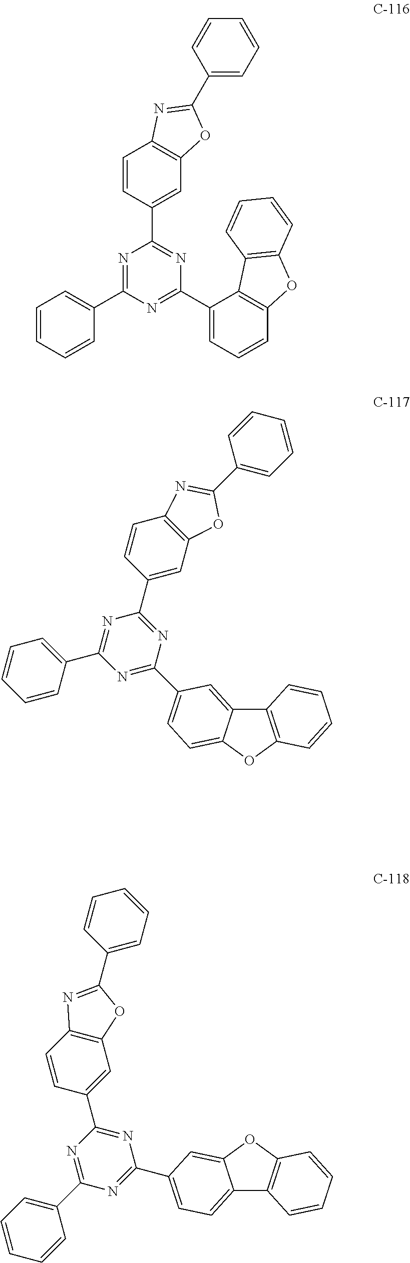 [SMILES: c1ccc(-c2nc(-c3ccc4nc(-c5ccccc5)oc4c3)nc(-c3cccc4oc5ccccc5c34)n2)cc1 ; c1ccc(-c2nc(-c3ccc4nc(-c5ccccc5)oc4c3)nc(-c3ccc4oc5ccccc5c4c3)n2)cc1 ; c1ccc(-c2nc(-c3ccc4nc(-c5ccccc5)oc4c3)nc(-c3ccc4c(c3)oc3ccccc34)n2)cc1]

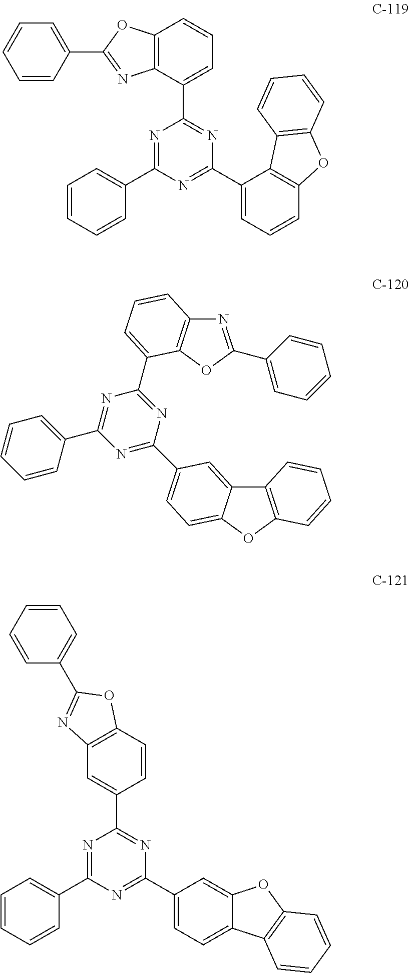 [SMILES: c1ccc(-c2nc(-c3cccc4oc(-c5ccccc5)nc34)nc(-c3cccc4oc5ccccc5c34)n2)cc1 ; c1ccc(-c2nc(-c3ccc4oc5ccccc5c4c3)nc(-c3cccc4nc(-c5ccccc5)oc34)n2)cc1 ; c1ccc(-c2nc(-c3ccc4oc(-c5ccccc5)nc4c3)nc(-c3ccc4c(c3)oc3ccccc34)n2)cc1]

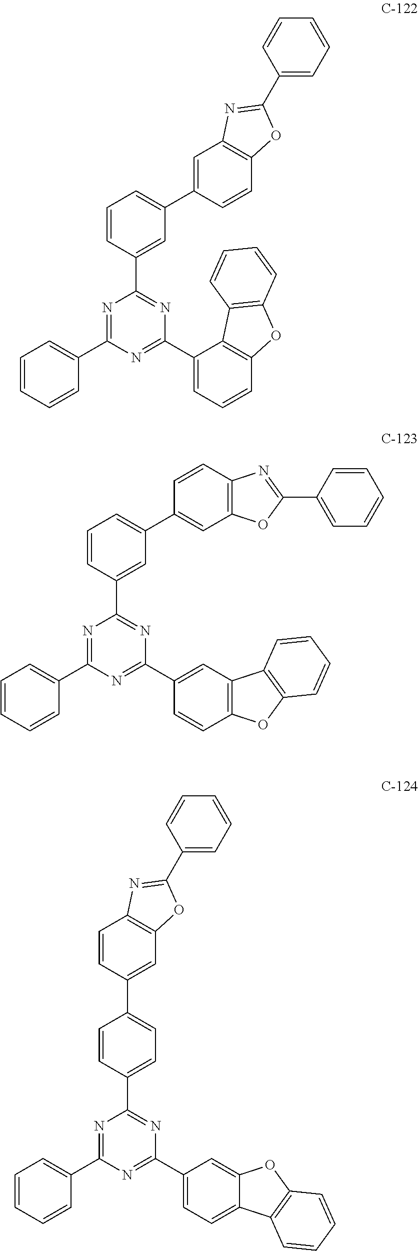 [SMILES: c1ccc(-c2nc(-c3cccc(-c4ccc5oc(-c6ccccc6)nc5c4)c3)nc(-c3cccc4oc5ccccc5c34)n2)cc1 ; c1ccc(-c2nc(-c3cccc(-c4ccc5nc(-c6ccccc6)oc5c4)c3)nc(-c3ccc4oc5ccccc5c4c3)n2)cc1 ; c1ccc(-c2nc(-c3ccc(-c4ccc5nc(-c6ccccc6)oc5c4)cc3)nc(-c3ccc4c(c3)oc3ccccc34)n2)cc1]

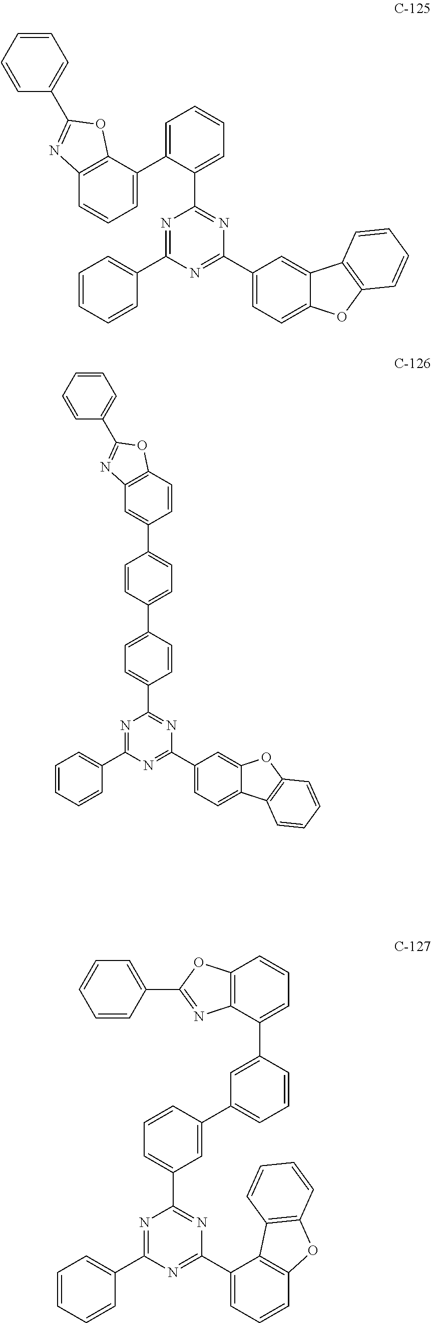 [SMILES: c1ccc(-c2nc(-c3ccc4oc5ccccc5c4c3)nc(-c3ccccc3-c3cccc4nc(-c5ccccc5)oc34)n2)cc1 ; c1ccc(-c2nc(-c3ccc(-c4ccc(-c5ccc6oc(-c7ccccc7)nc6c5)cc4)cc3)nc(-c3ccc4c(c3)oc3ccccc34)n2)cc1 ; c1ccc(-c2nc(-c3cccc(-c4cccc(-c5cccc6oc(-c7ccccc7)nc56)c4)c3)nc(-c3cccc4oc5ccccc5c34)n2)cc1]

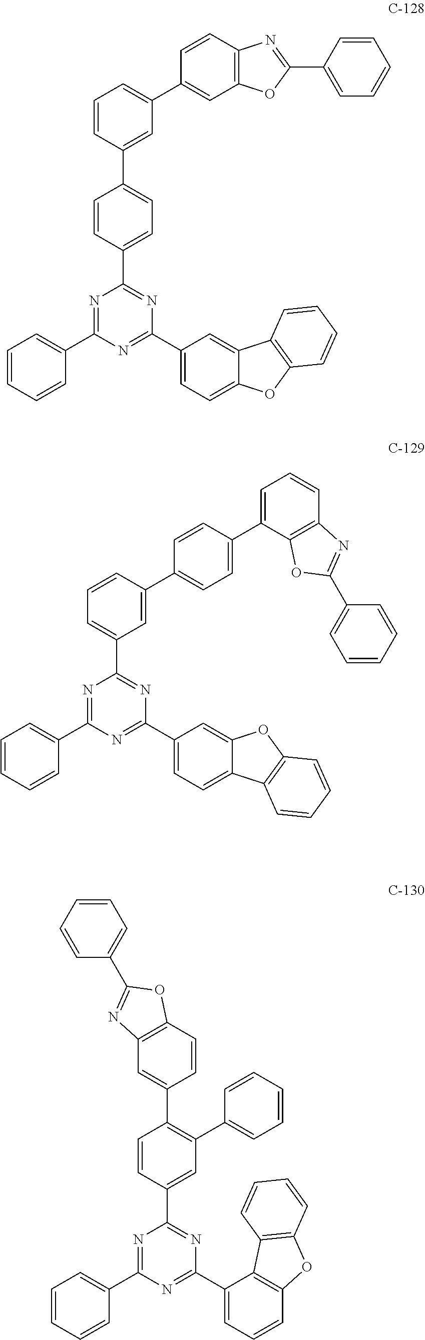 [SMILES: c1ccc(-c2nc(-c3ccc(-c4cccc(-c5ccc6nc(-c7ccccc7)oc6c5)c4)cc3)nc(-c3ccc4oc5ccccc5c4c3)n2)cc1 ; c1ccc(-c2nc(-c3cccc(-c4ccc(-c5cccc6nc(-c7ccccc7)oc56)cc4)c3)nc(-c3ccc4c(c3)oc3ccccc34)n2)cc1 ; c1ccc(-c2nc(-c3ccc(-c4ccc5oc(-c6ccccc6)nc5c4)c(-c4ccccc4)c3)nc(-c3cccc4oc5ccccc5c34)n2)cc1]

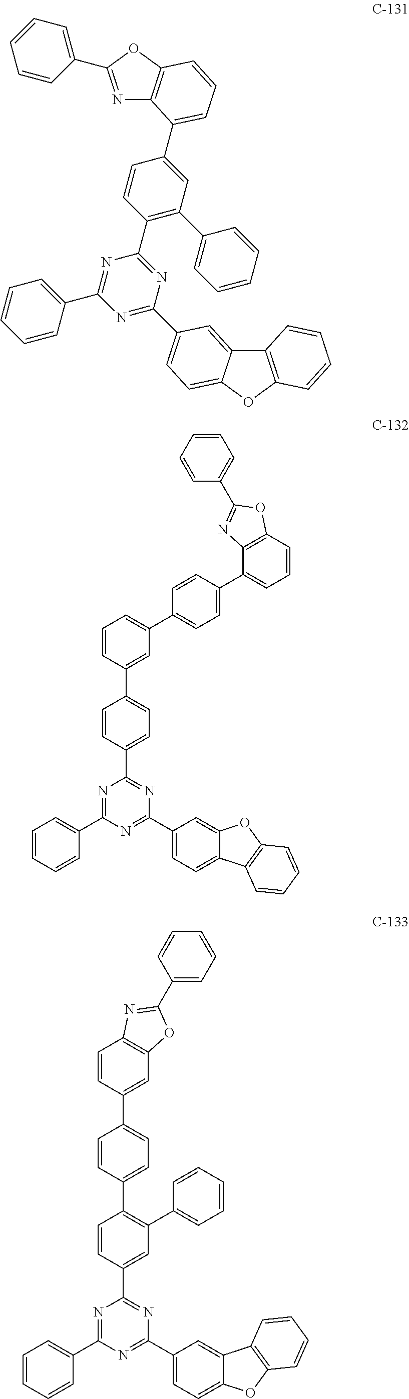 [SMILES: c1ccc(-c2nc(-c3ccc4oc5ccccc5c4c3)nc(-c3ccc(-c4cccc5oc(-c6ccccc6)nc45)cc3-c3ccccc3)n2)cc1 ; c1ccc(-c2nc(-c3ccc(-c4cccc(-c5ccc(-c6cccc7oc(-c8ccccc8)nc67)cc5)c4)cc3)nc(-c3ccc4c(c3)oc3ccccc34)n2)cc1 ; c1ccc(-c2nc(-c3ccc(-c4ccc(-c5ccc6nc(-c7ccccc7)oc6c5)cc4)c(-c4ccccc4)c3)nc(-c3ccc4oc5ccccc5c4c3)n2)cc1]

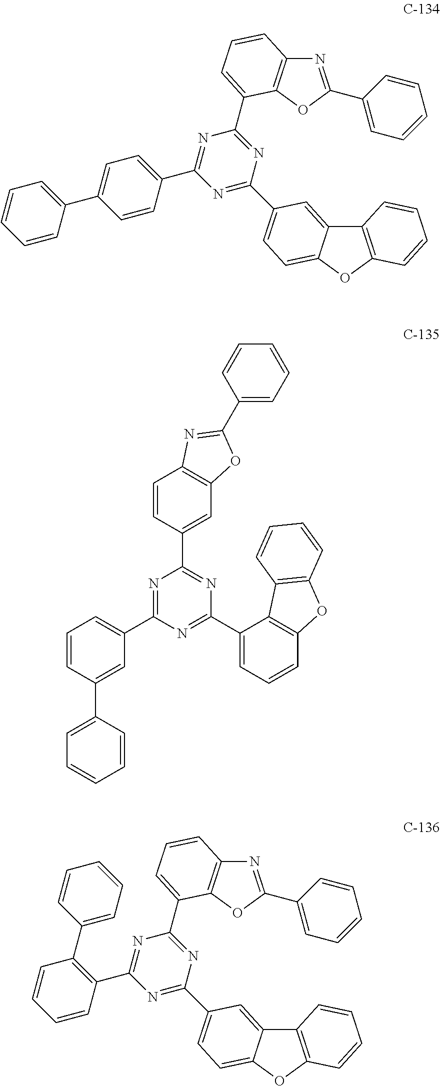 [SMILES: c1ccc(-c2ccc(-c3nc(-c4ccc5oc6ccccc6c5c4)nc(-c4cccc5nc(-c6ccccc6)oc45)n3)cc2)cc1 ; c1ccc(-c2cccc(-c3nc(-c4ccc5nc(-c6ccccc6)oc5c4)nc(-c4cccc5oc6ccccc6c45)n3)c2)cc1 ; c1ccc(-c2nc3cccc(-c4nc(-c5ccc6oc7ccccc7c6c5)nc(-c5ccccc5-c5ccccc5)n4)c3o2)cc1]

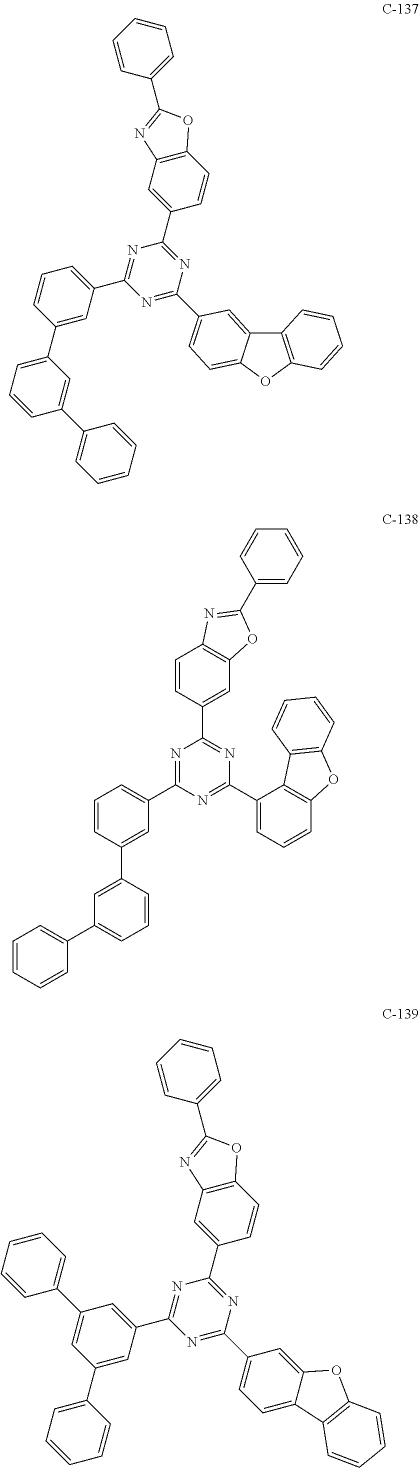 [SMILES: c1ccc(-c2cccc(-c3cccc(-c4nc(-c5ccc6oc(-c7ccccc7)nc6c5)nc(-c5ccc6oc7ccccc7c6c5)n4)c3)c2)cc1 ; c1ccc(-c2cccc(-c3cccc(-c4nc(-c5ccc6nc(-c7ccccc7)oc6c5)nc(-c5cccc6oc7ccccc7c56)n4)c3)c2)cc1 ; c1ccc(-c2cc(-c3ccccc3)cc(-c3nc(-c4ccc5oc(-c6ccccc6)nc5c4)nc(-c4ccc5c(c4)oc4ccccc45)n3)c2)cc1]

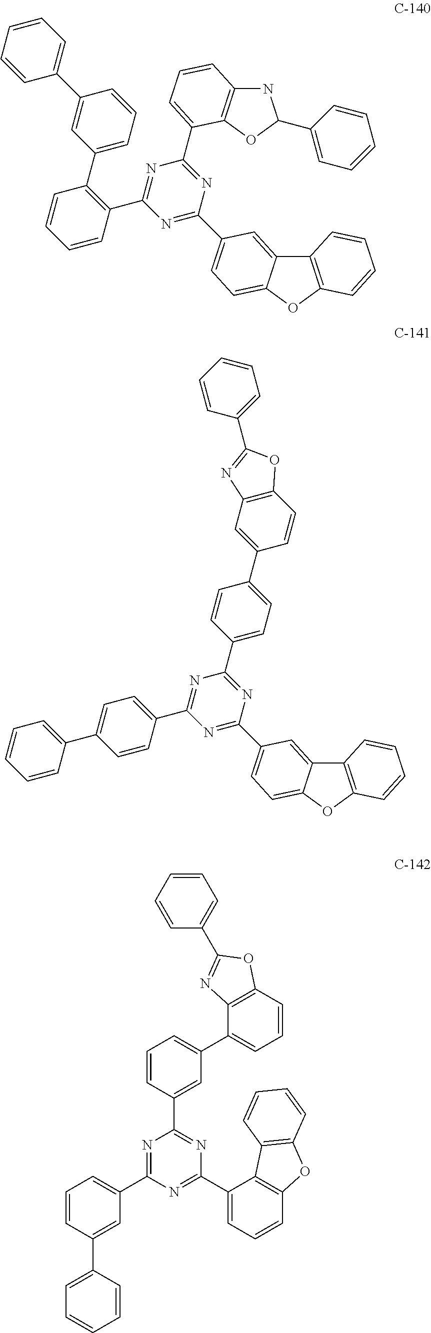 [SMILES: c1ccc(-c2cccc(-c3ccccc3-c3nc(-c4ccc5oc6ccccc6c5c4)nc(-c4cccc5c4OC(c4ccccc4)N5)n3)c2)cc1 ; c1ccc(-c2ccc(-c3nc(-c4ccc(-c5ccc6oc(-c7ccccc7)nc6c5)cc4)nc(-c4ccc5oc6ccccc6c5c4)n3)cc2)cc1 ; c1ccc(-c2cccc(-c3nc(-c4cccc(-c5cccc6oc(-c7ccccc7)nc56)c4)nc(-c4cccc5oc6ccccc6c45)n3)c2)cc1]

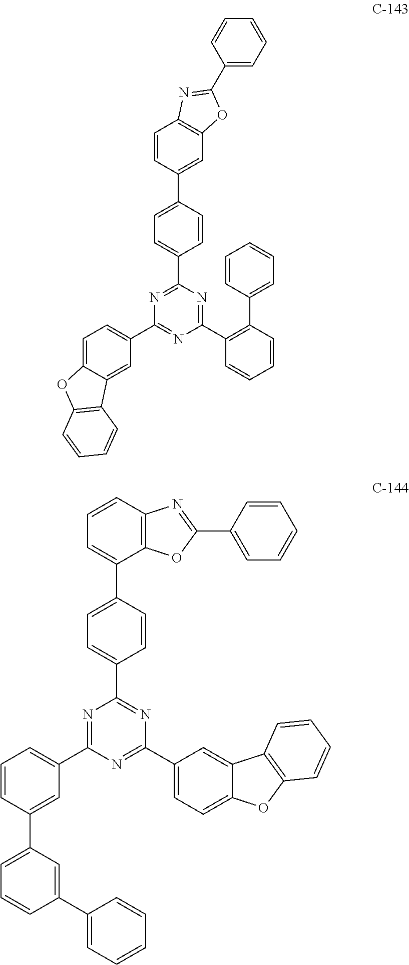

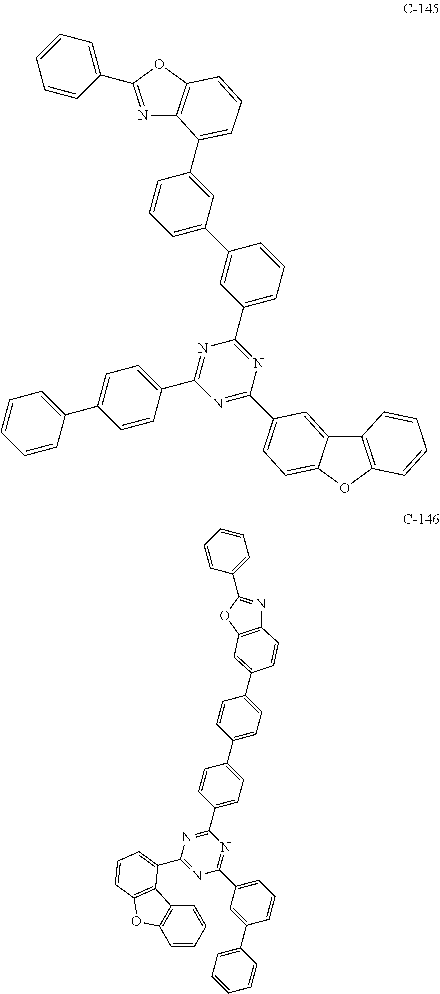 [SMILES: c1ccc(-c2ccc(-c3nc(-c4cccc(-c5cccc(-c6cccc7oc(-c8ccccc8)nc67)c5)c4)nc(-c4ccc5oc6ccccc6c5c4)n3)cc2)cc1 ; c1ccc(-c2cccc(-c3nc(-c4ccc(-c5ccc(-c6ccc7nc(-c8ccccc8)oc7c6)cc5)cc4)nc(-c4cccc5oc6ccccc6c45)n3)c2)cc1]

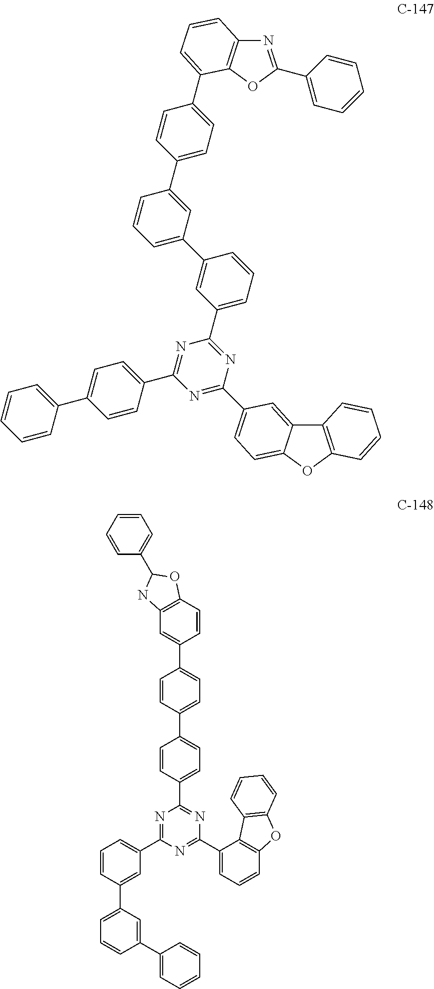 [SMILES: c1ccc(-c2ccc(-c3nc(-c4cccc(-c5cccc(-c6ccc(-c7cccc8nc(-c9ccccc9)oc78)cc6)c5)c4)nc(-c4ccc5oc6ccccc6c5c4)n3)cc2)cc1 ; c1ccc(-c2cccc(-c3cccc(-c4nc(-c5ccc(-c6ccc(-c7ccc8c(c7)NC(c7ccccc7)O8)cc6)cc5)nc(-c5cccc6oc7ccccc7c56)n4)c3)c2)cc1]

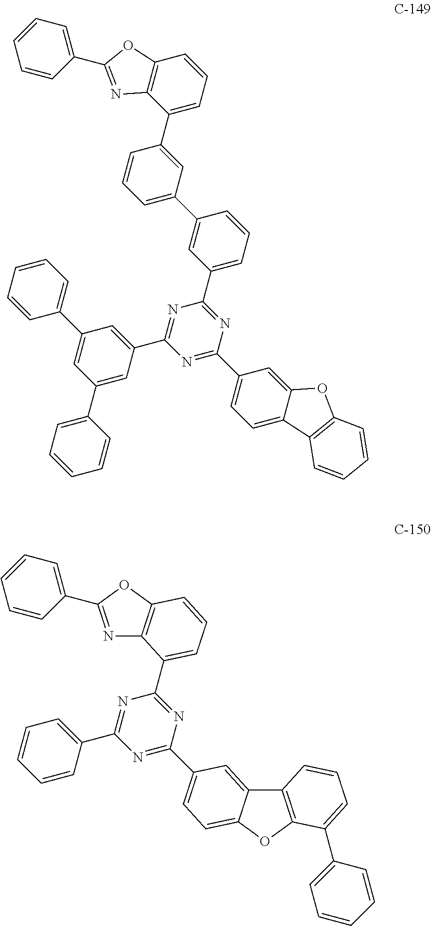 [SMILES: c1ccc(-c2cc(-c3ccccc3)cc(-c3nc(-c4cccc(-c5cccc(-c6cccc7oc(-c8ccccc8)nc67)c5)c4)nc(-c4ccc5c(c4)oc4ccccc45)n3)c2)cc1 ; c1ccc(-c2nc(-c3ccc4oc5c(-c6ccccc6)cccc5c4c3)nc(-c3cccc4oc(-c5ccccc5)nc34)n2)cc1]

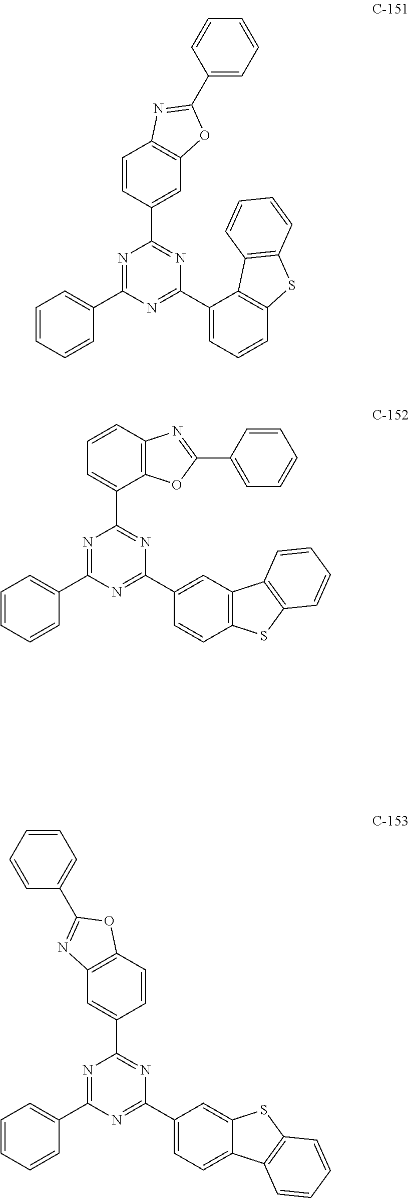 [SMILES: c1ccc(-c2nc(-c3ccc4nc(-c5ccccc5)oc4c3)nc(-c3cccc4sc5ccccc5c34)n2)cc1 ; c1ccc(-c2nc(-c3ccc4sc5ccccc5c4c3)nc(-c3cccc4nc(-c5ccccc5)oc34)n2)cc1 ; c1ccc(-c2nc(-c3ccc4oc(-c5ccccc5)nc4c3)nc(-c3ccc4c(c3)sc3ccccc34)n2)cc1]

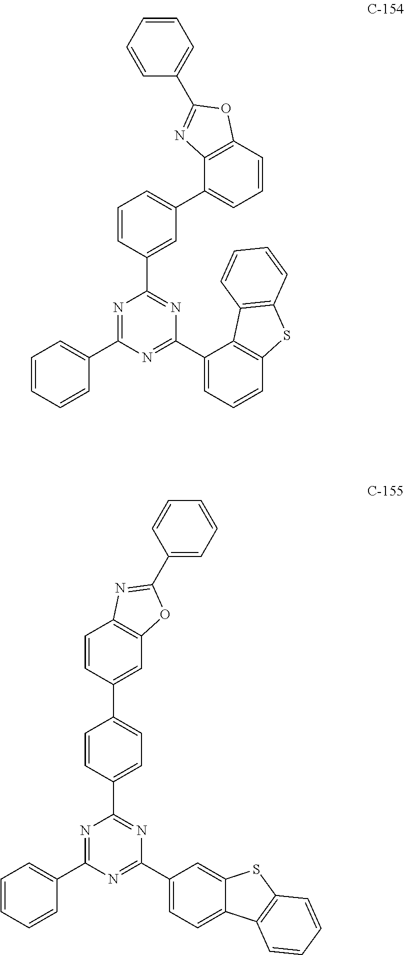 [SMILES: c1ccc(-c2nc(-c3cccc(-c4cccc5oc(-c6ccccc6)nc45)c3)nc(-c3cccc4sc5ccccc5c34)n2)cc1 ; c1ccc(-c2nc(-c3ccc(-c4ccc5nc(-c6ccccc6)oc5c4)cc3)nc(-c3ccc4c(c3)sc3ccccc34)n2)cc1]

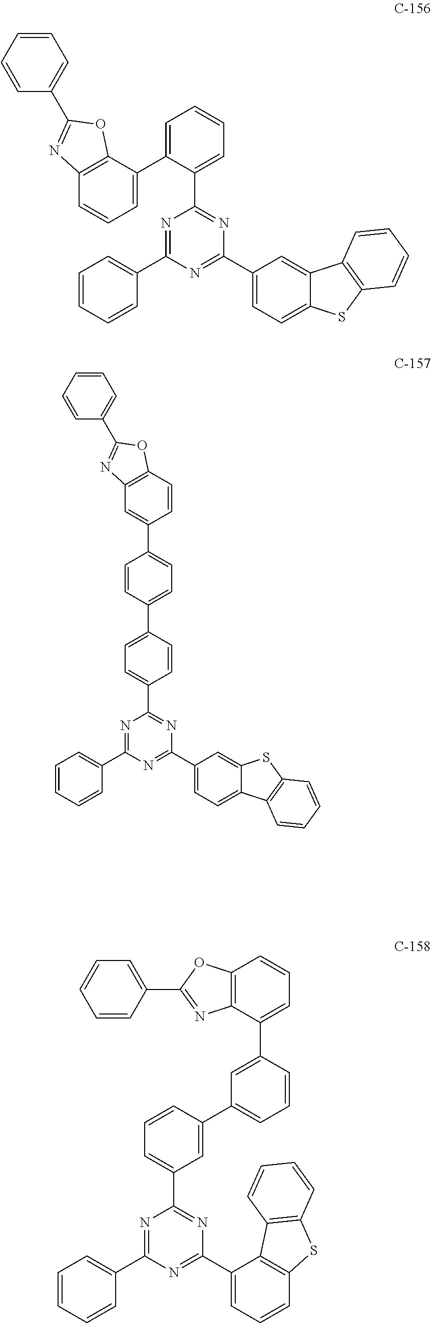 [SMILES: c1ccc(-c2nc(-c3ccc4sc5ccccc5c4c3)nc(-c3ccccc3-c3cccc4nc(-c5ccccc5)oc34)n2)cc1 ; c1ccc(-c2nc(-c3ccc(-c4ccc(-c5ccc6oc(-c7ccccc7)nc6c5)cc4)cc3)nc(-c3ccc4c(c3)sc3ccccc34)n2)cc1 ; c1ccc(-c2nc(-c3cccc(-c4cccc(-c5cccc6oc(-c7ccccc7)nc56)c4)c3)nc(-c3cccc4sc5ccccc5c34)n2)cc1]

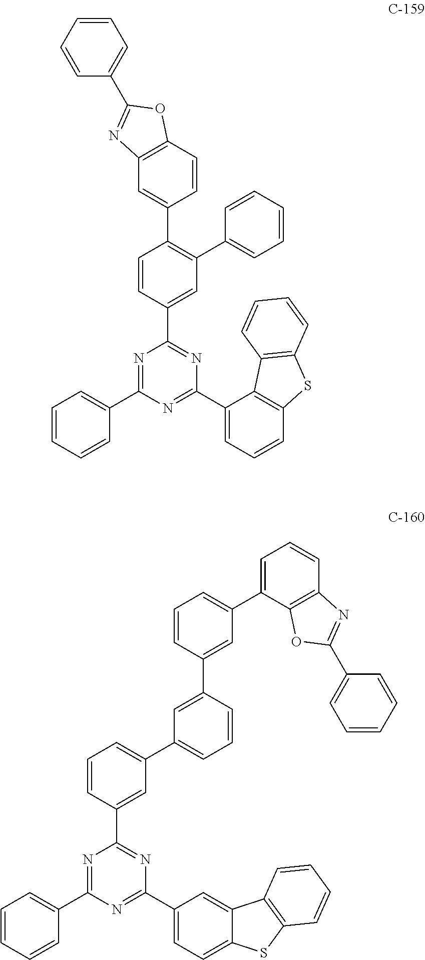 [SMILES: c1ccc(-c2nc(-c3ccc(-c4ccc5oc(-c6ccccc6)nc5c4)c(-c4ccccc4)c3)nc(-c3cccc4sc5ccccc5c34)n2)cc1 ; c1ccc(-c2nc(-c3cccc(-c4cccc(-c5cccc(-c6cccc7nc(-c8ccccc8)oc67)c5)c4)c3)nc(-c3ccc4sc5ccccc5c4c3)n2)cc1]

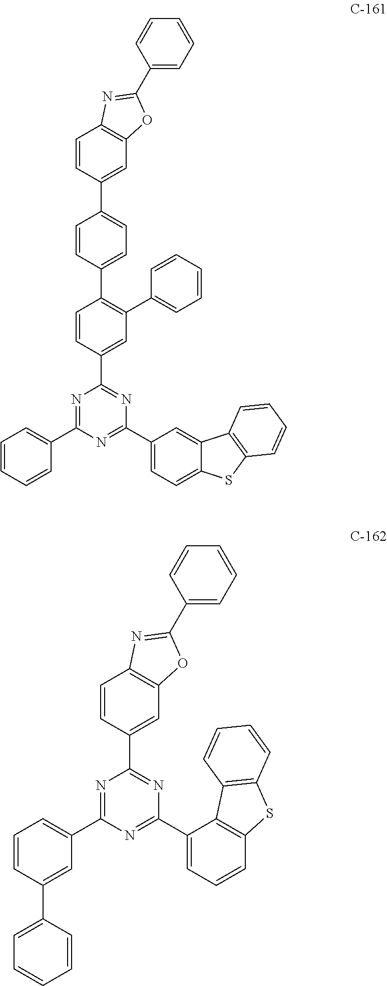 [SMILES: c1ccc(-c2nc(-c3ccc(-c4ccc(-c5ccc6nc(-c7ccccc7)oc6c5)cc4)c(-c4ccccc4)c3)nc(-c3ccc4sc5ccccc5c4c3)n2)cc1 ; c1ccc(-c2cccc(-c3nc(-c4ccc5nc(-c6ccccc6)oc5c4)nc(-c4cccc5sc6ccccc6c45)n3)c2)cc1]

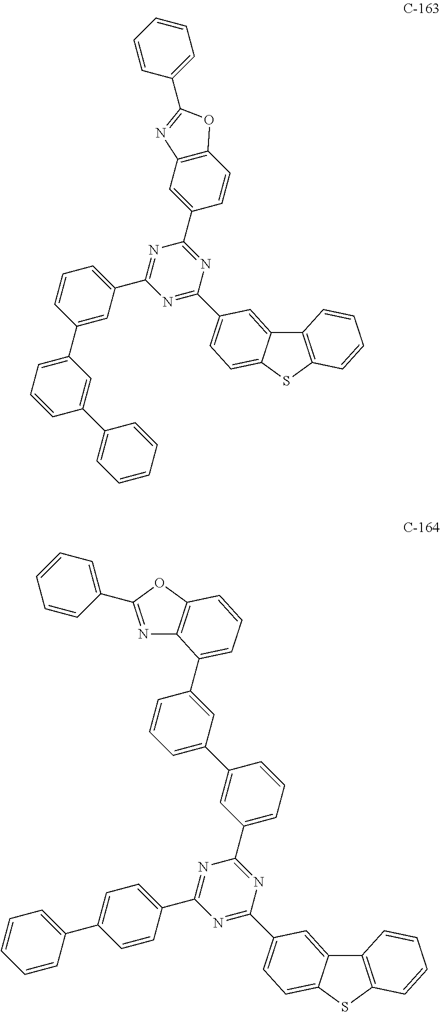 [SMILES: c1ccc(-c2cccc(-c3cccc(-c4nc(-c5ccc6oc(-c7ccccc7)nc6c5)nc(-c5ccc6sc7ccccc7c6c5)n4)c3)c2)cc1 ; c1ccc(-c2ccc(-c3nc(-c4cccc(-c5cccc(-c6cccc7oc(-c8ccccc8)nc67)c5)c4)nc(-c4ccc5sc6ccccc6c5c4)n3)cc2)cc1]

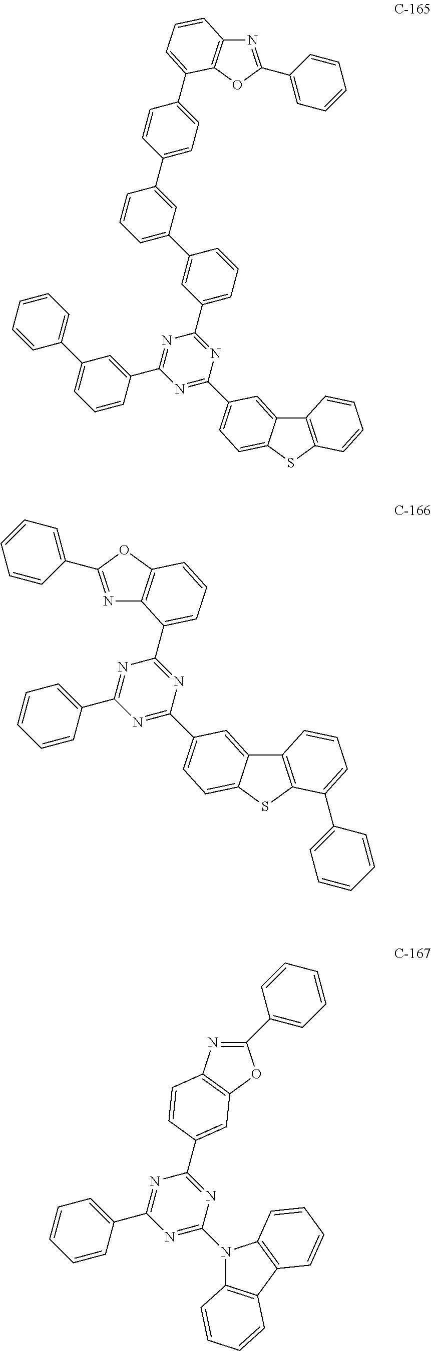 [SMILES: c1ccc(-c2cccc(-c3nc(-c4cccc(-c5cccc(-c6ccc(-c7cccc8nc(-c9ccccc9)oc78)cc6)c5)c4)nc(-c4ccc5sc6ccccc6c5c4)n3)c2)cc1 ; c1ccc(-c2nc(-c3ccc4sc5c(-c6ccccc6)cccc5c4c3)nc(-c3cccc4oc(-c5ccccc5)nc34)n2)cc1 ; c1ccc(-c2nc(-c3ccc4nc(-c5ccccc5)oc4c3)nc(-n3c4ccccc4c4ccccc43)n2)cc1]

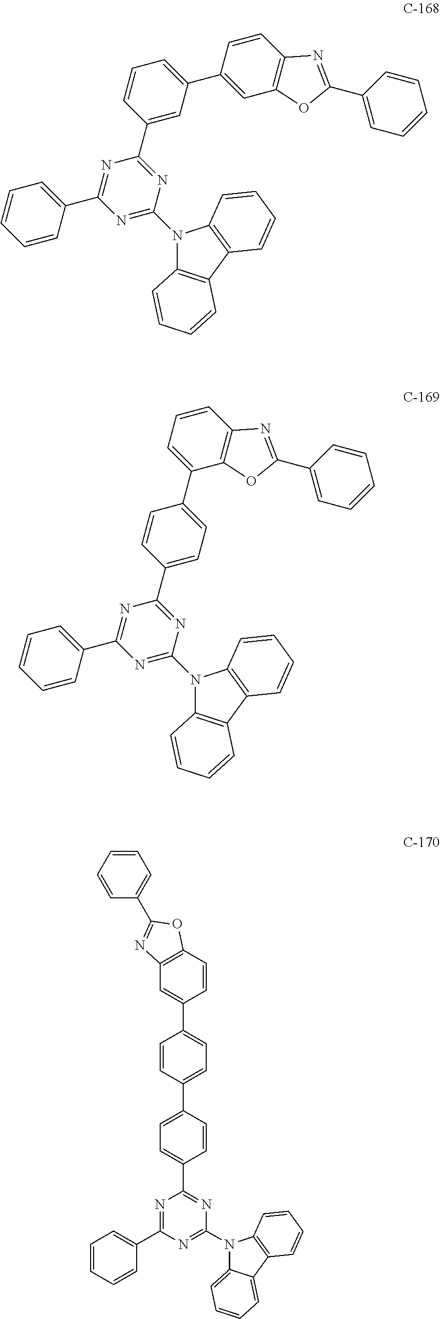 [SMILES: c1ccc(-c2nc(-c3cccc(-c4ccc5nc(-c6ccccc6)oc5c4)c3)nc(-n3c4ccccc4c4ccccc43)n2)cc1 ; c1ccc(-c2nc(-c3ccc(-c4cccc5nc(-c6ccccc6)oc45)cc3)nc(-n3c4ccccc4c4ccccc43)n2)cc1 ; c1ccc(-c2nc(-c3ccc(-c4ccc(-c5ccc6oc(-c7ccccc7)nc6c5)cc4)cc3)nc(-n3c4ccccc4c4ccccc43)n2)cc1]

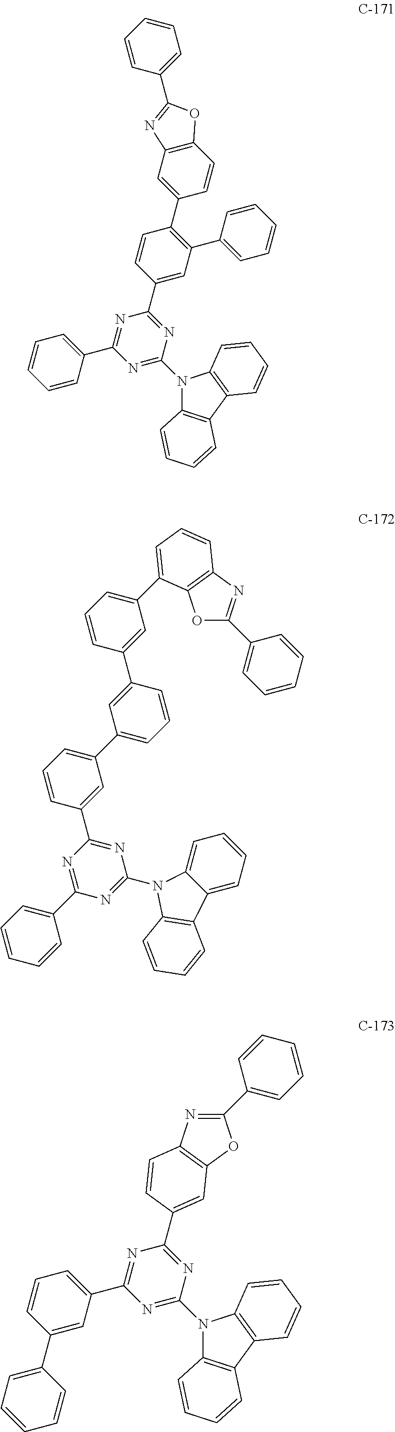 [SMILES: c1ccc(-c2nc(-c3ccc(-c4ccc5oc(-c6ccccc6)nc5c4)c(-c4ccccc4)c3)nc(-n3c4ccccc4c4ccccc43)n2)cc1 ; c1ccc(-c2nc(-c3cccc(-c4cccc(-c5cccc(-c6cccc7nc(-c8ccccc8)oc67)c5)c4)c3)nc(-n3c4ccccc4c4ccccc43)n2)cc1 ; c1ccc(-c2cccc(-c3nc(-c4ccc5nc(-c6ccccc6)oc5c4)nc(-n4c5ccccc5c5ccccc54)n3)c2)cc1]

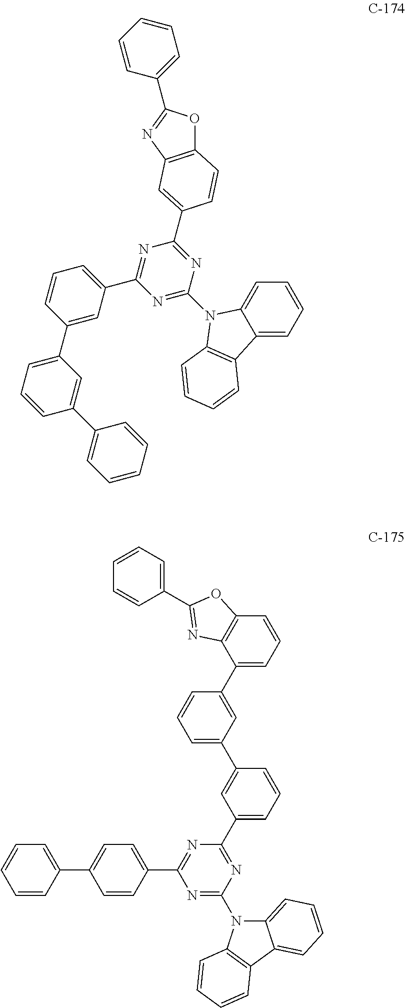 [SMILES: c1ccc(-c2cccc(-c3cccc(-c4nc(-c5ccc6oc(-c7ccccc7)nc6c5)nc(-n5c6ccccc6c6ccccc65)n4)c3)c2)cc1 ; c1ccc(-c2ccc(-c3nc(-c4cccc(-c5cccc(-c6cccc7oc(-c8ccccc8)nc67)c5)c4)nc(-n4c5ccccc5c5ccccc54)n3)cc2)cc1]

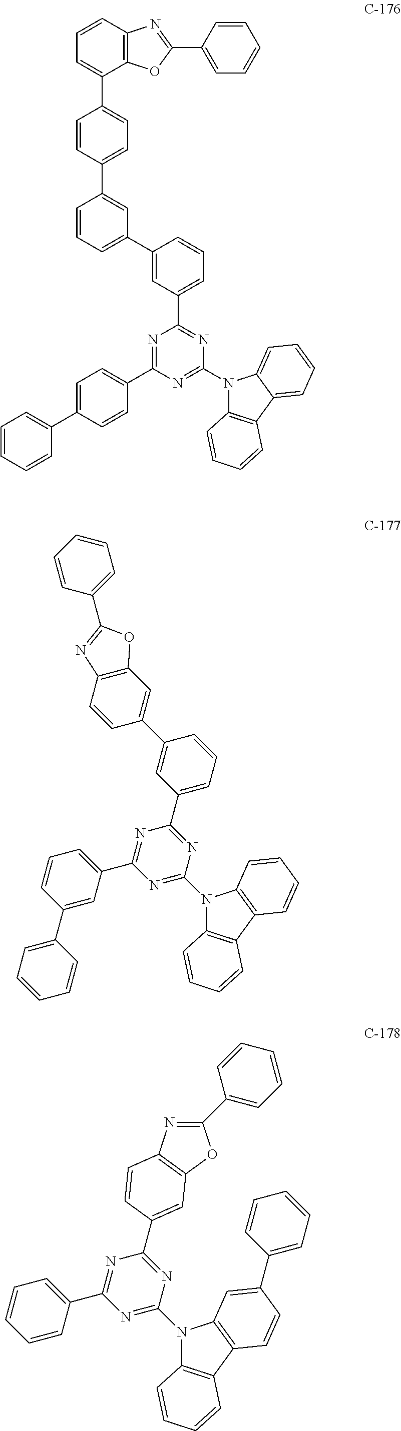 [SMILES: c1ccc(-c2ccc(-c3nc(-c4cccc(-c5cccc(-c6ccc(-c7cccc8nc(-c9ccccc9)oc78)cc6)c5)c4)nc(-n4c5ccccc5c5ccccc54)n3)cc2)cc1 ; c1ccc(-c2cccc(-c3nc(-c4cccc(-c5ccc6nc(-c7ccccc7)oc6c5)c4)nc(-n4c5ccccc5c5ccccc54)n3)c2)cc1 ; c1ccc(-c2ccc3c4ccccc4n(-c4nc(-c5ccccc5)nc(-c5ccc6nc(-c7ccccc7)oc6c5)n4)c3c2)cc1]

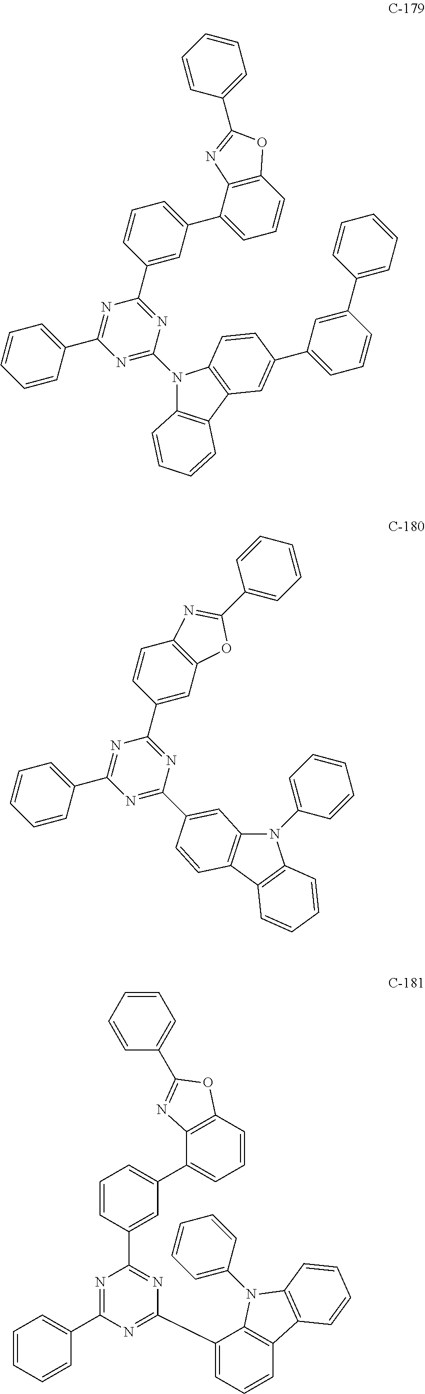 [SMILES: c1ccc(-c2cccc(-c3ccc4c(c3)c3ccccc3n4-c3nc(-c4ccccc4)nc(-c4cccc(-c5cccc6oc(-c7ccccc7)nc56)c4)n3)c2)cc1 ; c1ccc(-c2nc(-c3ccc4nc(-c5ccccc5)oc4c3)nc(-c3ccc4c5ccccc5n(-c5ccccc5)c4c3)n2)cc1 ; c1ccc(-c2nc(-c3cccc(-c4cccc5oc(-c6ccccc6)nc45)c3)nc(-c3cccc4c5ccccc5n(-c5ccccc5)c34)n2)cc1]

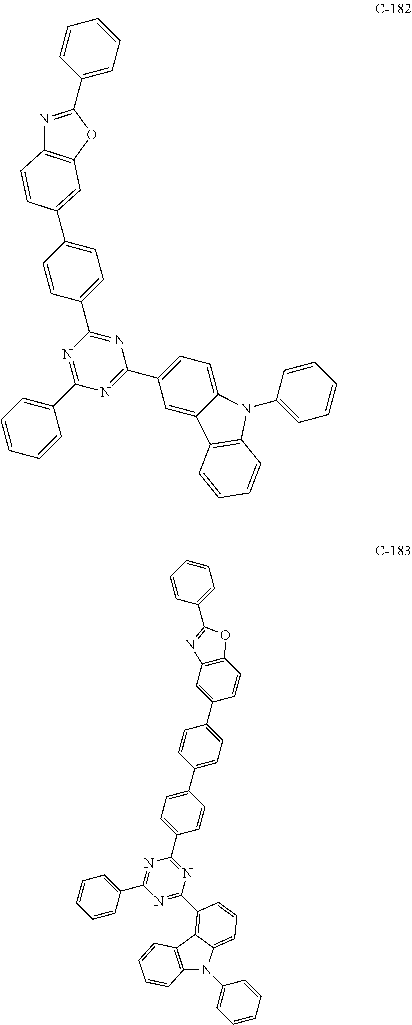 [SMILES: c1ccc(-c2nc(-c3ccc(-c4ccc5nc(-c6ccccc6)oc5c4)cc3)nc(-c3ccc4c(c3)c3ccccc3n4-c3ccccc3)n2)cc1 ; c1ccc(-c2nc(-c3ccc(-c4ccc(-c5ccc6oc(-c7ccccc7)nc6c5)cc4)cc3)nc(-c3cccc4c3c3ccccc3n4-c3ccccc3)n2)cc1]

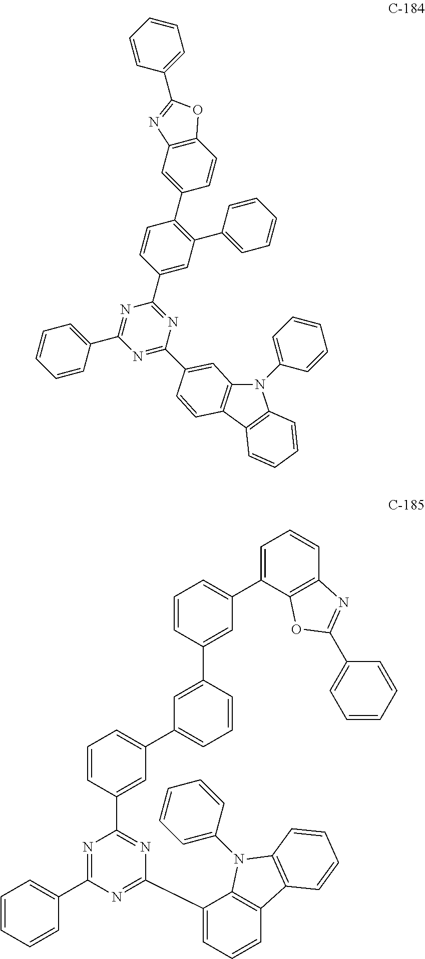 [SMILES: c1ccc(-c2nc(-c3ccc(-c4ccc5oc(-c6ccccc6)nc5c4)c(-c4ccccc4)c3)nc(-c3ccc4c5ccccc5n(-c5ccccc5)c4c3)n2)cc1 ; c1ccc(-c2nc(-c3cccc(-c4cccc(-c5cccc(-c6cccc7nc(-c8ccccc8)oc67)c5)c4)c3)nc(-c3cccc4c5ccccc5n(-c5ccccc5)c34)n2)cc1]

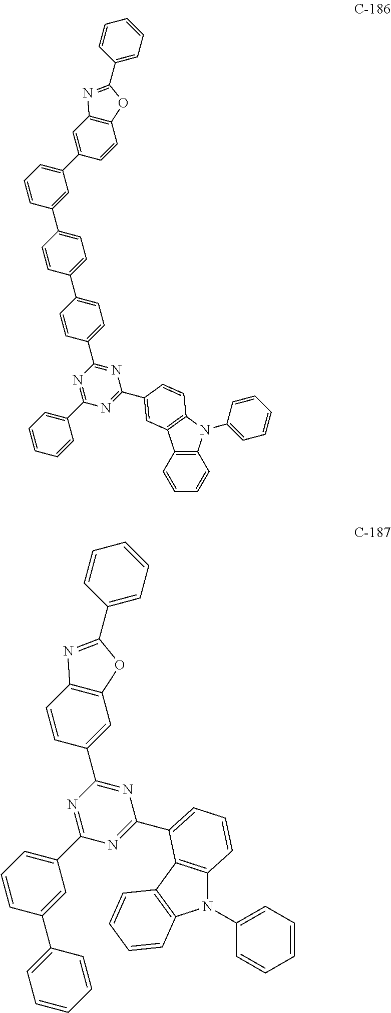 [SMILES: c1ccc(-c2nc(-c3ccc(-c4ccc(-c5cccc(-c6ccc7oc(-c8ccccc8)nc7c6)c5)cc4)cc3)nc(-c3ccc4c(c3)c3ccccc3n4-c3ccccc3)n2)cc1 ; c1ccc(-c2cccc(-c3nc(-c4ccc5nc(-c6ccccc6)oc5c4)nc(-c4cccc5c4c4ccccc4n5-c4ccccc4)n3)c2)cc1]

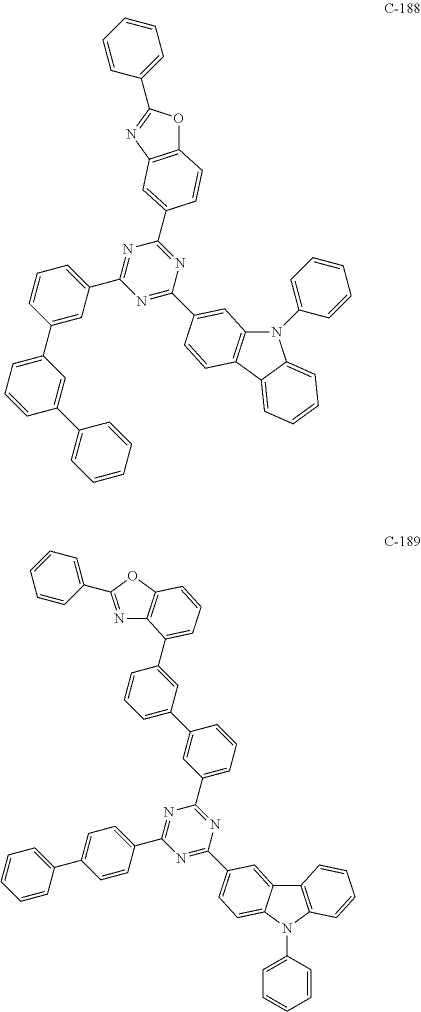 [SMILES: c1ccc(-c2cccc(-c3cccc(-c4nc(-c5ccc6oc(-c7ccccc7)nc6c5)nc(-c5ccc6c7ccccc7n(-c7ccccc7)c6c5)n4)c3)c2)cc1 ; c1ccc(-c2ccc(-c3nc(-c4cccc(-c5cccc(-c6cccc7oc(-c8ccccc8)nc67)c5)c4)nc(-c4ccc5c(c4)c4ccccc4n5-c4ccccc4)n3)cc2)cc1]

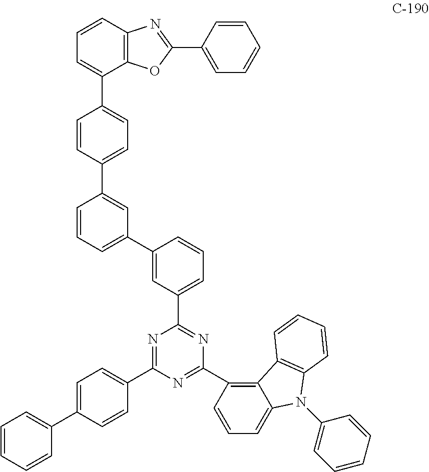 [SMILES: c1ccc(-c2ccc(-c3nc(-c4cccc(-c5cccc(-c6ccc(-c7cccc8nc(-c9ccccc9)oc78)cc6)c5)c4)nc(-c4cccc5c4c4ccccc4n5-c4ccccc4)n3)cc2)cc1]

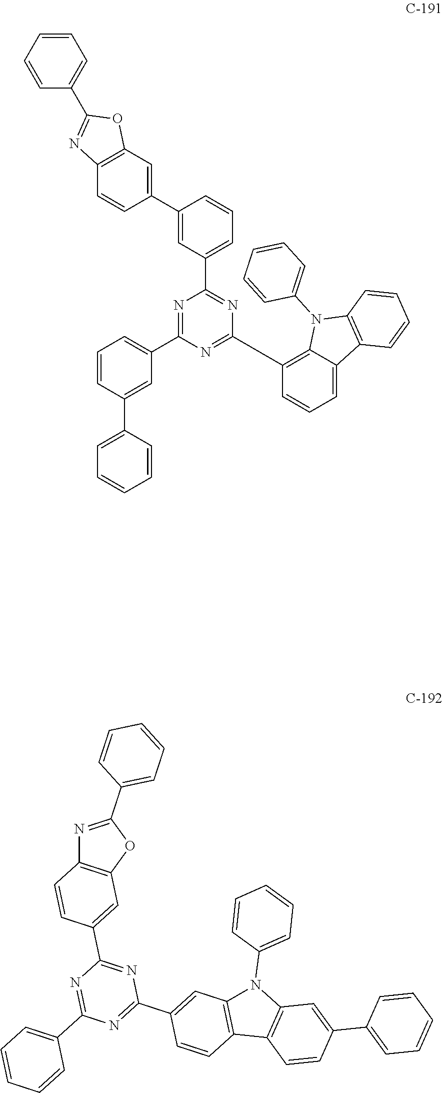 [SMILES: c1ccc(-c2cccc(-c3nc(-c4cccc(-c5ccc6nc(-c7ccccc7)oc6c5)c4)nc(-c4cccc5c6ccccc6n(-c6ccccc6)c45)n3)c2)cc1 ; c1ccc(-c2ccc3c4ccc(-c5nc(-c6ccccc6)nc(-c6ccc7nc(-c8ccccc8)oc7c6)n5)cc4n(-c4ccccc4)c3c2)cc1]

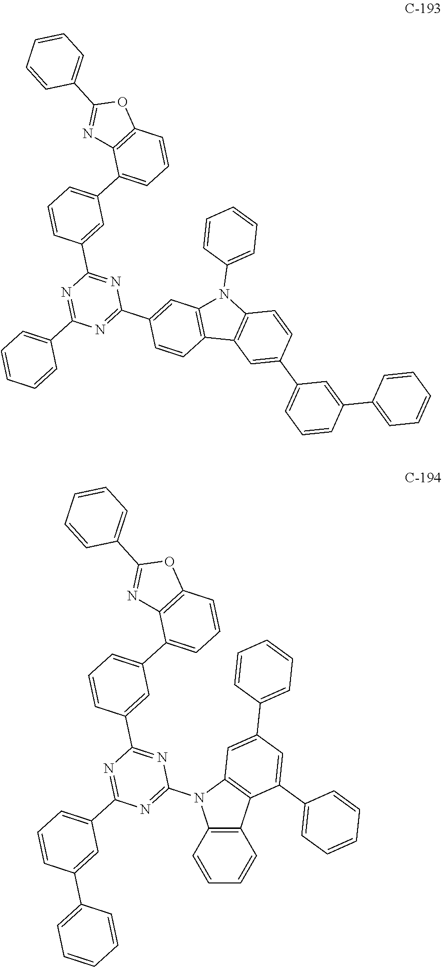 [SMILES: c1ccc(-c2cccc(-c3ccc4c(c3)c3ccc(-c5nc(-c6ccccc6)nc(-c6cccc(-c7cccc8oc(-c9ccccc9)nc78)c6)n5)cc3n4-c3ccccc3)c2)cc1 ; c1ccc(-c2cccc(-c3nc(-c4cccc(-c5cccc6oc(-c7ccccc7)nc56)c4)nc(-n4c5ccccc5c5c(-c6ccccc6)cc(-c6ccccc6)cc54)n3)c2)cc1]

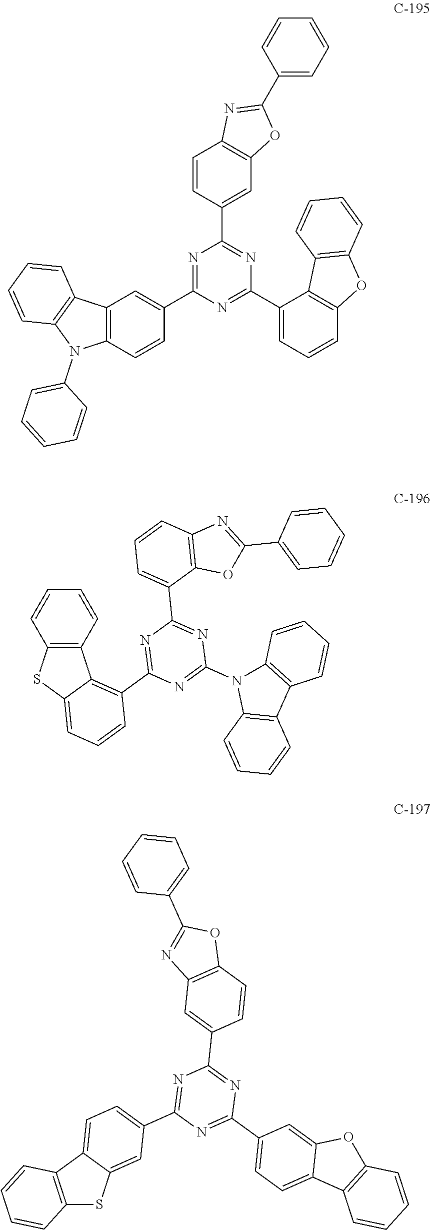 [SMILES: c1ccc(-c2nc3ccc(-c4nc(-c5ccc6c(c5)c5ccccc5n6-c5ccccc5)nc(-c5cccc6oc7ccccc7c56)n4)cc3o2)cc1 ; c1ccc(-c2nc3cccc(-c4nc(-c5cccc6sc7ccccc7c56)nc(-n5c6ccccc6c6ccccc65)n4)c3o2)cc1 ; c1ccc(-c2nc3cc(-c4nc(-c5ccc6c(c5)oc5ccccc56)nc(-c5ccc6c(c5)sc5ccccc56)n4)ccc3o2)cc1]

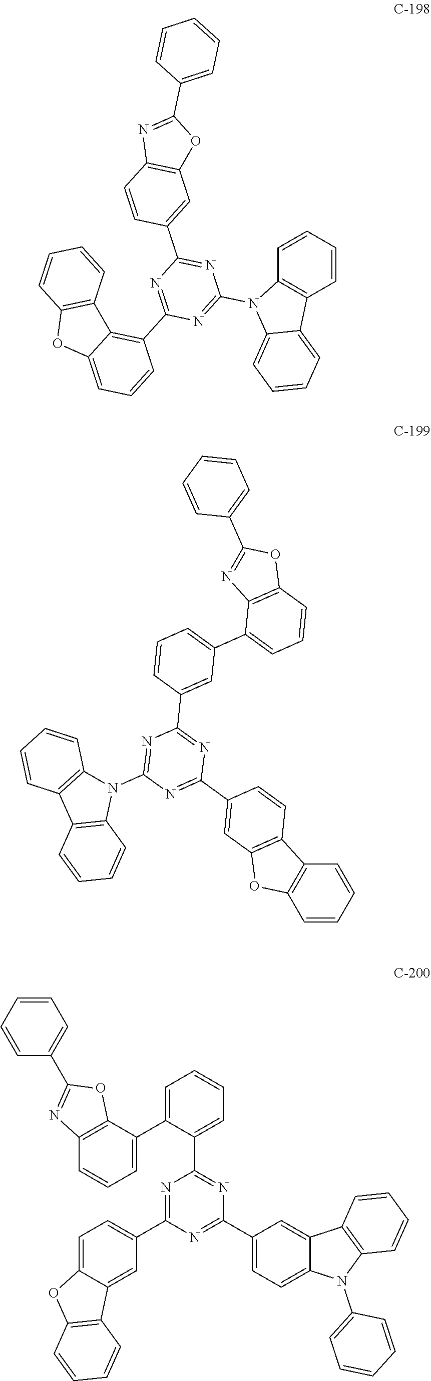 [SMILES: c1ccc(-c2nc3ccc(-c4nc(-c5cccc6oc7ccccc7c56)nc(-n5c6ccccc6c6ccccc65)n4)cc3o2)cc1 ; c1ccc(-c2nc3c(-c4cccc(-c5nc(-c6ccc7c(c6)oc6ccccc67)nc(-n6c7ccccc7c7ccccc76)n5)c4)cccc3o2)cc1 ; c1ccc(-c2nc3cccc(-c4ccccc4-c4nc(-c5ccc6oc7ccccc7c6c5)nc(-c5ccc6c(c5)c5ccccc5n6-c5ccccc5)n4)c3o2)cc1]

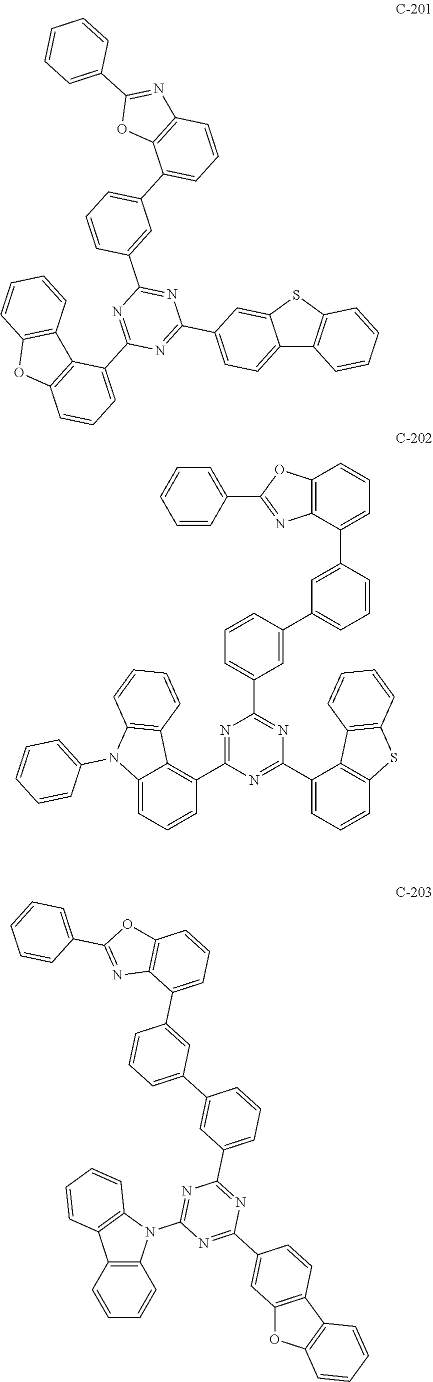 [SMILES: c1ccc(-c2nc3cccc(-c4cccc(-c5nc(-c6ccc7c(c6)sc6ccccc67)nc(-c6cccc7oc8ccccc8c67)n5)c4)c3o2)cc1 ; c1ccc(-c2nc3c(-c4cccc(-c5cccc(-c6nc(-c7cccc8sc9ccccc9c78)nc(-c7cccc8c7c7ccccc7n8-c7ccccc7)n6)c5)c4)cccc3o2)cc1 ; c1ccc(-c2nc3c(-c4cccc(-c5cccc(-c6nc(-c7ccc8c(c7)oc7ccccc78)nc(-n7c8ccccc8c8ccccc87)n6)c5)c4)cccc3o2)cc1]

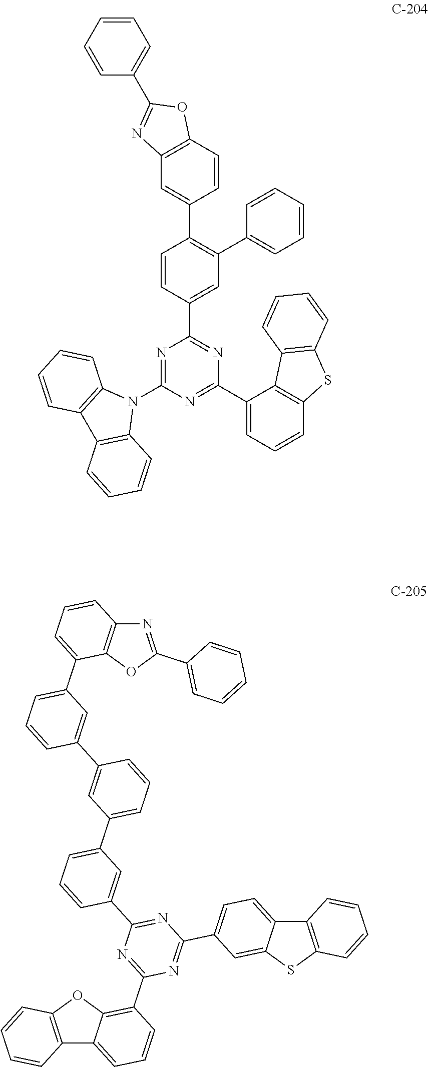 [SMILES: c1ccc(-c2nc3cc(-c4ccc(-c5nc(-c6cccc7sc8ccccc8c67)nc(-n6c7ccccc7c7ccccc76)n5)cc4-c4ccccc4)ccc3o2)cc1 ; c1ccc(-c2nc3cccc(-c4cccc(-c5cccc(-c6cccc(-c7nc(-c8ccc9c(c8)sc8ccccc89)nc(-c8cccc9c8oc8ccccc89)n7)c6)c5)c4)c3o2)cc1]

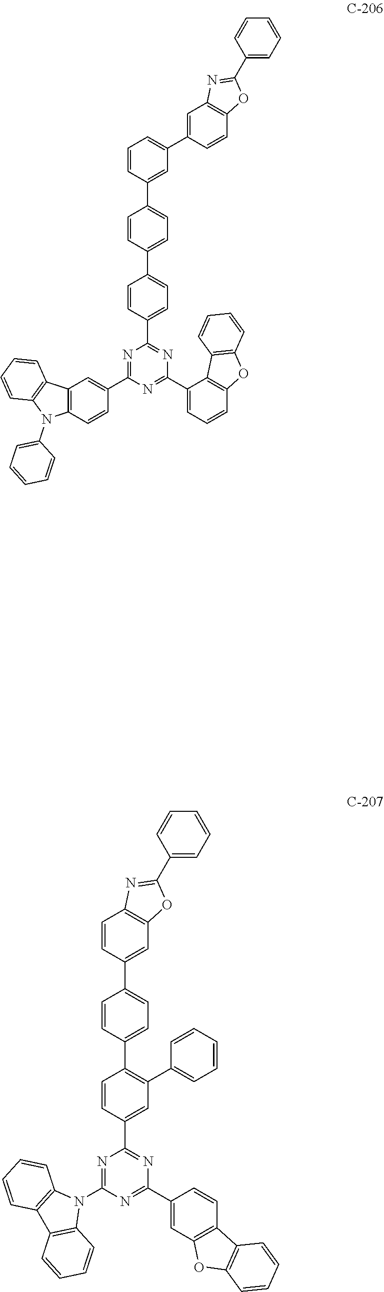 [SMILES: c1ccc(-c2nc3cc(-c4cccc(-c5ccc(-c6ccc(-c7nc(-c8ccc9c(c8)c8ccccc8n9-c8ccccc8)nc(-c8cccc9oc%10ccccc%10c89)n7)cc6)cc5)c4)ccc3o2)cc1 ; c1ccc(-c2nc3ccc(-c4ccc(-c5ccc(-c6nc(-c7ccc8c(c7)oc7ccccc78)nc(-n7c8ccccc8c8ccccc87)n6)cc5-c5ccccc5)cc4)cc3o2)cc1]

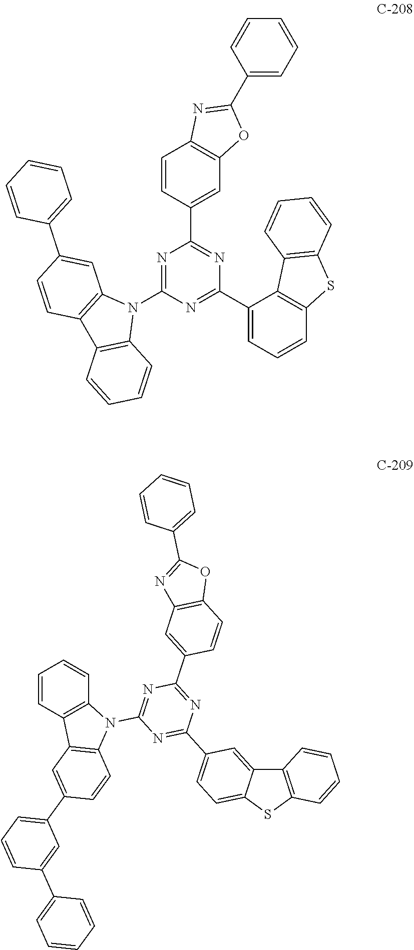 [SMILES: c1ccc(-c2ccc3c4ccccc4n(-c4nc(-c5ccc6nc(-c7ccccc7)oc6c5)nc(-c5cccc6sc7ccccc7c56)n4)c3c2)cc1 ; c1ccc(-c2cccc(-c3ccc4c(c3)c3ccccc3n4-c3nc(-c4ccc5oc(-c6ccccc6)nc5c4)nc(-c4ccc5sc6ccccc6c5c4)n3)c2)cc1]

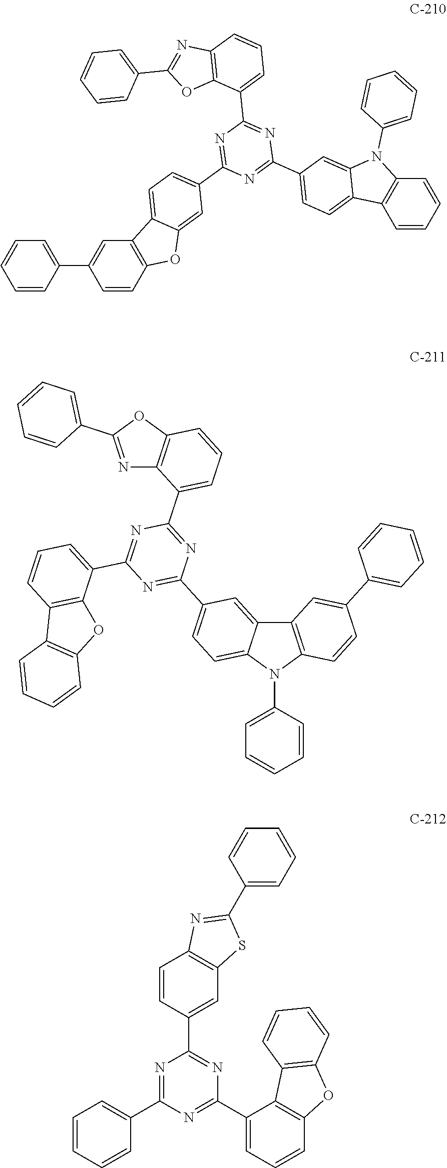 [SMILES: c1ccc(-c2ccc3oc4cc(-c5nc(-c6ccc7c8ccccc8n(-c8ccccc8)c7c6)nc(-c6cccc7nc(-c8ccccc8)oc67)n5)ccc4c3c2)cc1 ; c1ccc(-c2ccc3c(c2)c2cc(-c4nc(-c5cccc6oc(-c7ccccc7)nc56)nc(-c5cccc6c5oc5ccccc56)n4)ccc2n3-c2ccccc2)cc1 ; c1ccc(-c2nc(-c3ccc4nc(-c5ccccc5)sc4c3)nc(-c3cccc4oc5ccccc5c34)n2)cc1]

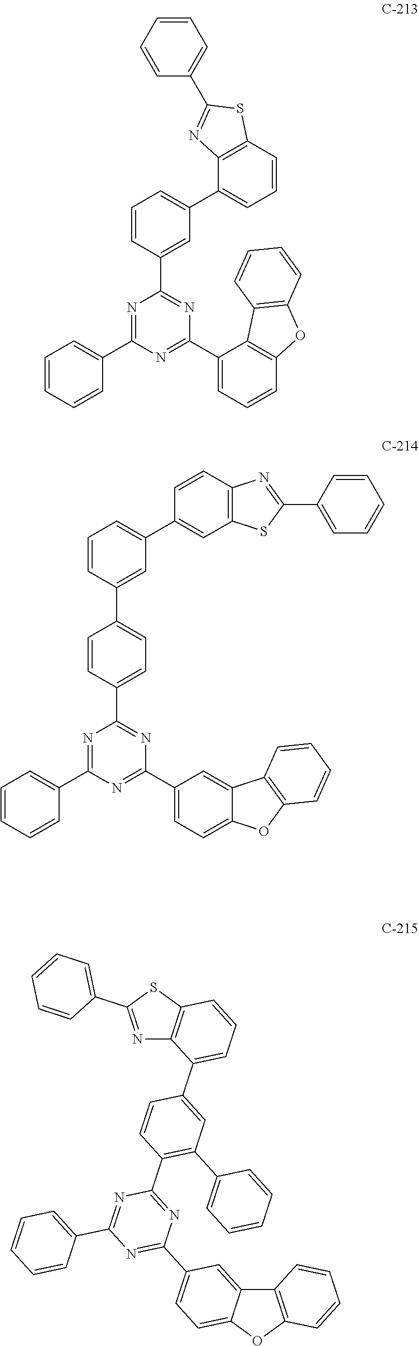 [SMILES: c1ccc(-c2nc(-c3cccc(-c4cccc5sc(-c6ccccc6)nc45)c3)nc(-c3cccc4oc5ccccc5c34)n2)cc1 ; c1ccc(-c2nc(-c3ccc(-c4cccc(-c5ccc6nc(-c7ccccc7)sc6c5)c4)cc3)nc(-c3ccc4oc5ccccc5c4c3)n2)cc1 ; c1ccc(-c2nc(-c3ccc4oc5ccccc5c4c3)nc(-c3ccc(-c4cccc5sc(-c6ccccc6)nc45)cc3-c3ccccc3)n2)cc1]

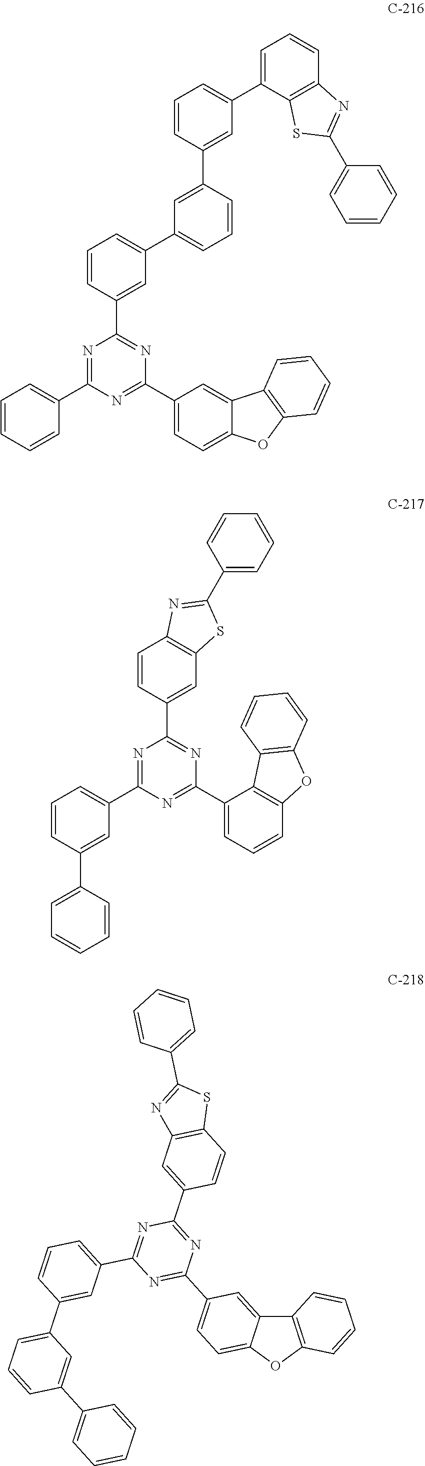 [SMILES: c1ccc(-c2nc(-c3cccc(-c4cccc(-c5cccc(-c6cccc7nc(-c8ccccc8)sc67)c5)c4)c3)nc(-c3ccc4oc5ccccc5c4c3)n2)cc1 ; c1ccc(-c2cccc(-c3nc(-c4ccc5nc(-c6ccccc6)sc5c4)nc(-c4cccc5oc6ccccc6c45)n3)c2)cc1 ; c1ccc(-c2cccc(-c3cccc(-c4nc(-c5ccc6sc(-c7ccccc7)nc6c5)nc(-c5ccc6oc7ccccc7c6c5)n4)c3)c2)cc1]

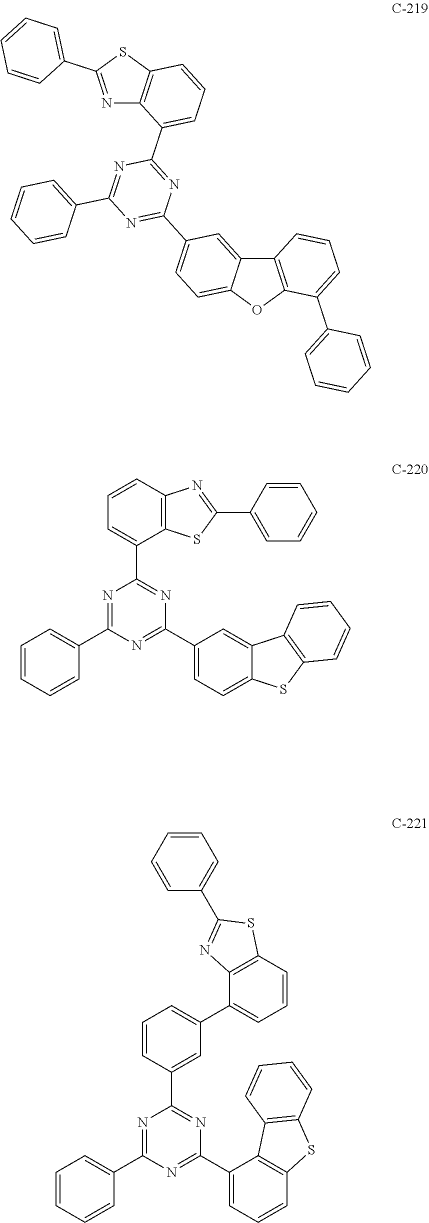 [SMILES: c1ccc(-c2nc(-c3ccc4oc5c(-c6ccccc6)cccc5c4c3)nc(-c3cccc4sc(-c5ccccc5)nc34)n2)cc1 ; c1ccc(-c2nc(-c3ccc4sc5ccccc5c4c3)nc(-c3cccc4nc(-c5ccccc5)sc34)n2)cc1 ; c1ccc(-c2nc(-c3cccc(-c4cccc5sc(-c6ccccc6)nc45)c3)nc(-c3cccc4sc5ccccc5c34)n2)cc1]

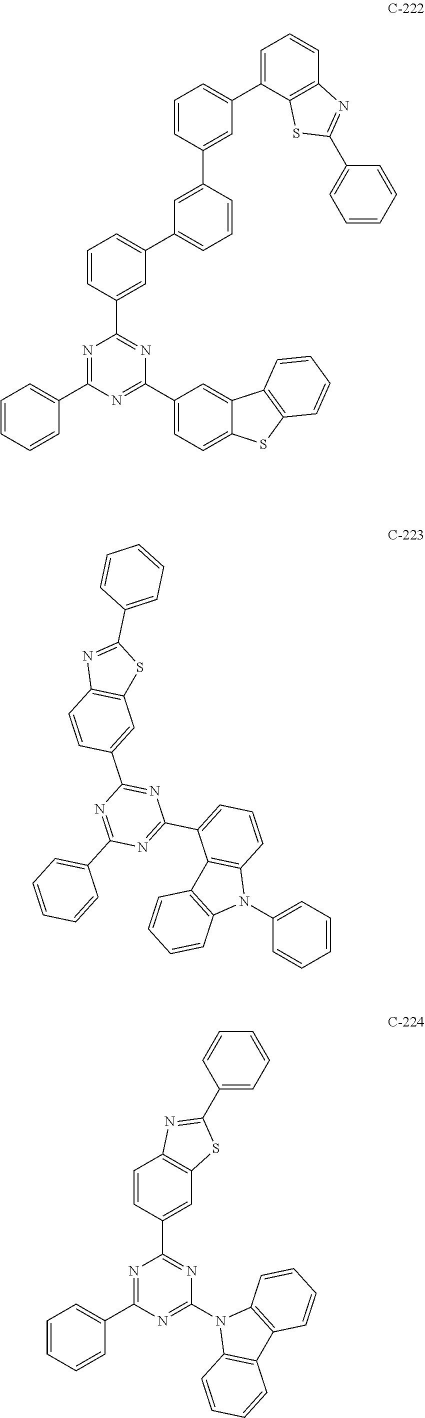 [SMILES: c1ccc(-c2nc(-c3cccc(-c4cccc(-c5cccc(-c6cccc7nc(-c8ccccc8)sc67)c5)c4)c3)nc(-c3ccc4sc5ccccc5c4c3)n2)cc1 ; c1ccc(-c2nc(-c3ccc4nc(-c5ccccc5)sc4c3)nc(-c3cccc4c3c3ccccc3n4-c3ccccc3)n2)cc1 ; c1ccc(-c2nc(-c3ccc4nc(-c5ccccc5)sc4c3)nc(-n3c4ccccc4c4ccccc43)n2)cc1]

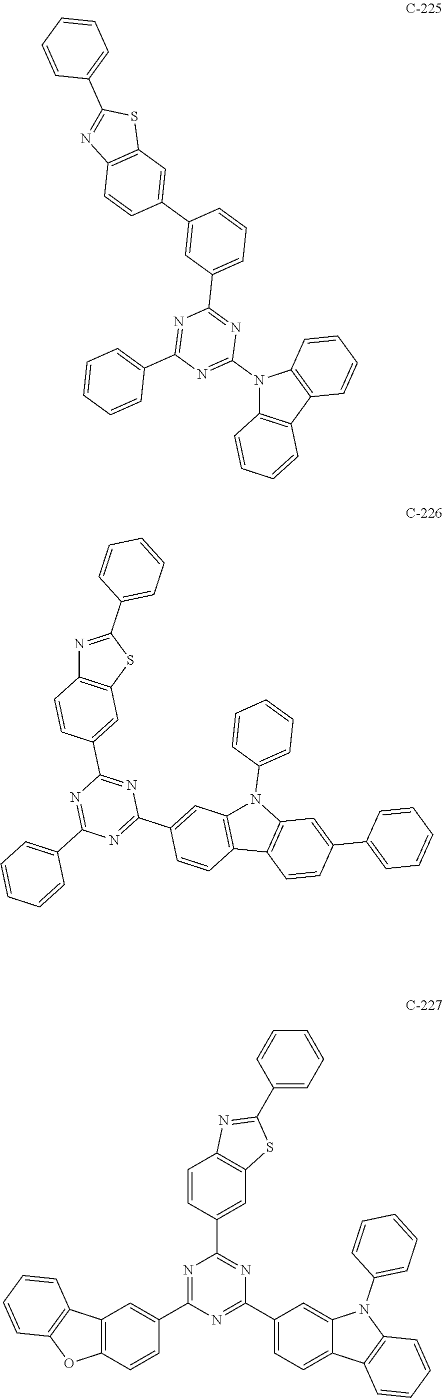 [SMILES: c1ccc(-c2nc(-c3cccc(-c4ccc5nc(-c6ccccc6)sc5c4)c3)nc(-n3c4ccccc4c4ccccc43)n2)cc1 ; c1ccc(-c2ccc3c4ccc(-c5nc(-c6ccccc6)nc(-c6ccc7nc(-c8ccccc8)sc7c6)n5)cc4n(-c4ccccc4)c3c2)cc1 ; c1ccc(-c2nc3ccc(-c4nc(-c5ccc6oc7ccccc7c6c5)nc(-c5ccc6c7ccccc7n(-c7ccccc7)c6c5)n4)cc3s2)cc1]

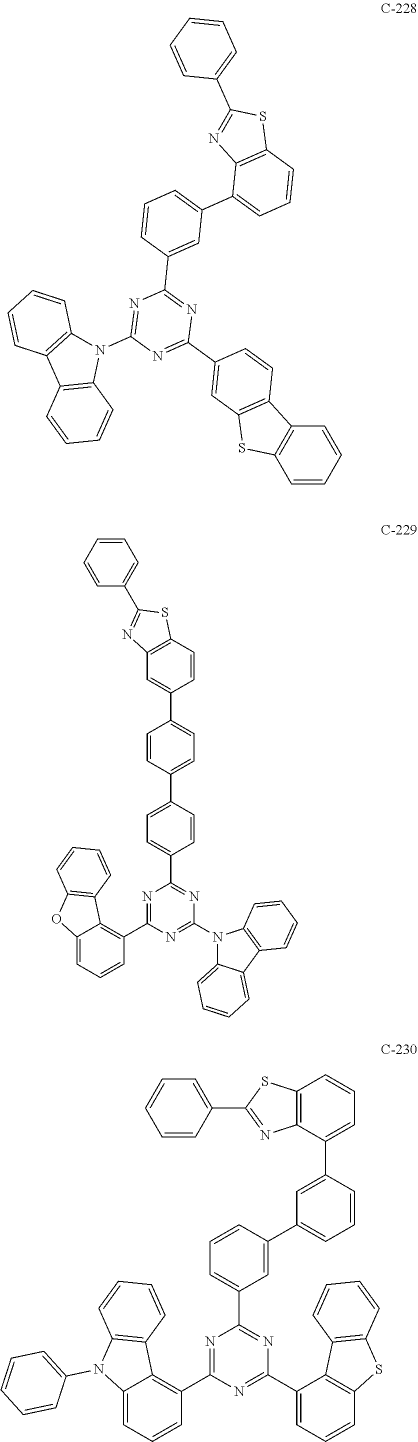 [SMILES: c1ccc(-c2nc3c(-c4cccc(-c5nc(-c6ccc7c(c6)sc6ccccc67)nc(-n6c7ccccc7c7ccccc76)n5)c4)cccc3s2)cc1 ; c1ccc(-c2nc3cc(-c4ccc(-c5ccc(-c6nc(-c7cccc8oc9ccccc9c78)nc(-n7c8ccccc8c8ccccc87)n6)cc5)cc4)ccc3s2)cc1 ; c1ccc(-c2nc3c(-c4cccc(-c5cccc(-c6nc(-c7cccc8sc9ccccc9c78)nc(-c7cccc8c7c7ccccc7n8-c7ccccc7)n6)c5)c4)cccc3s2)cc1]

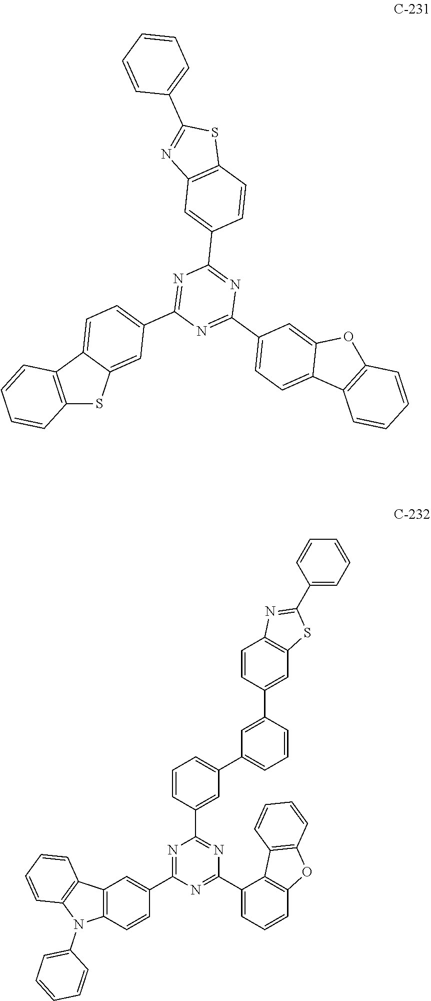 [SMILES: c1ccc(-c2nc3cc(-c4nc(-c5ccc6c(c5)oc5ccccc56)nc(-c5ccc6c(c5)sc5ccccc56)n4)ccc3s2)cc1 ; c1ccc(-c2nc3ccc(-c4cccc(-c5cccc(-c6nc(-c7ccc8c(c7)c7ccccc7n8-c7ccccc7)nc(-c7cccc8oc9ccccc9c78)n6)c5)c4)cc3s2)cc1]

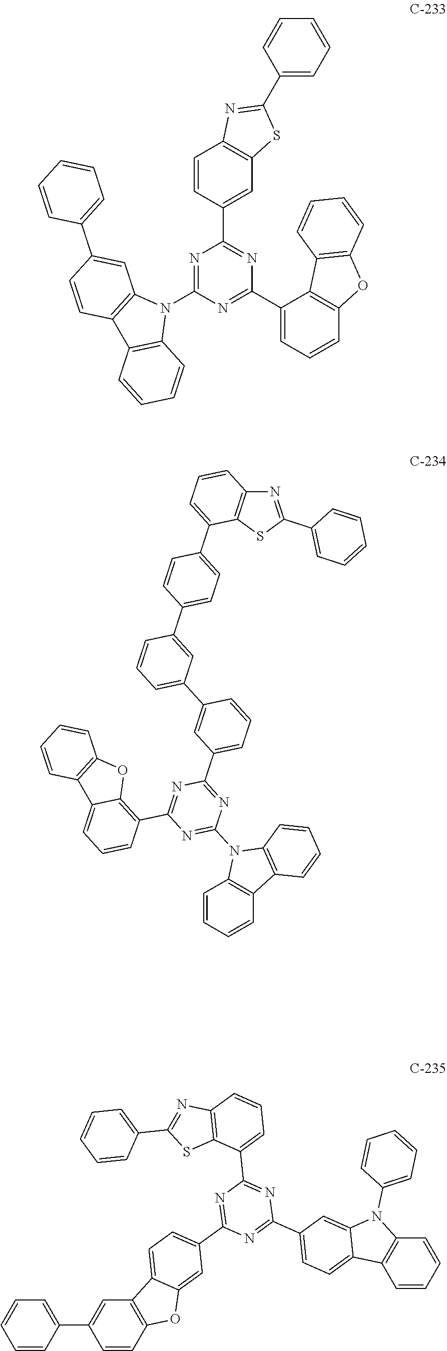 [SMILES: c1ccc(-c2ccc3c4ccccc4n(-c4nc(-c5ccc6nc(-c7ccccc7)sc6c5)nc(-c5cccc6oc7ccccc7c56)n4)c3c2)cc1 ; c1ccc(-c2nc3cccc(-c4ccc(-c5cccc(-c6cccc(-c7nc(-c8cccc9c8oc8ccccc89)nc(-n8c9ccccc9c9ccccc98)n7)c6)c5)cc4)c3s2)cc1 ; c1ccc(-c2ccc3oc4cc(-c5nc(-c6ccc7c8ccccc8n(-c8ccccc8)c7c6)nc(-c6cccc7nc(-c8ccccc8)sc67)n5)ccc4c3c2)cc1]

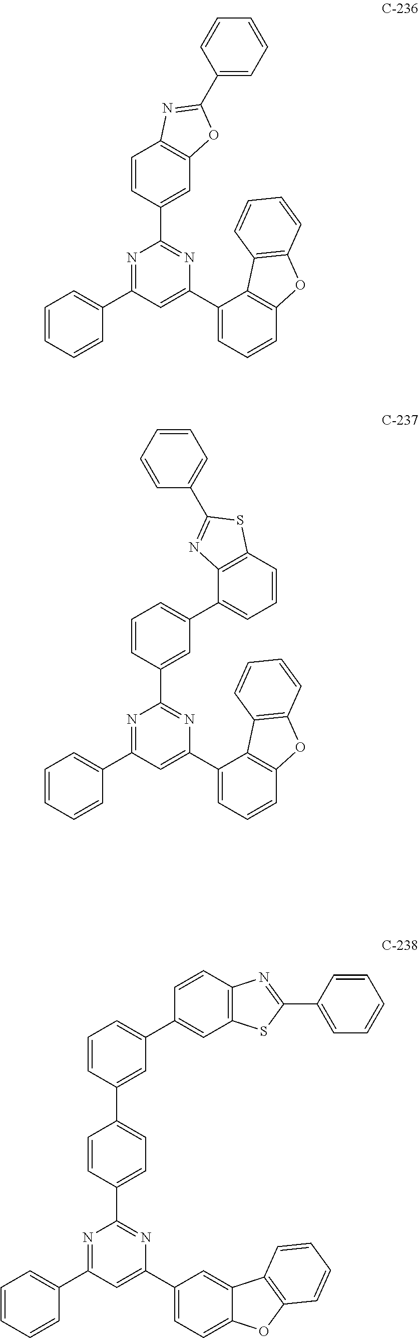 [SMILES: c1ccc(-c2cc(-c3cccc4oc5ccccc5c34)nc(-c3ccc4nc(-c5ccccc5)oc4c3)n2)cc1 ; c1ccc(-c2cc(-c3cccc4oc5ccccc5c34)nc(-c3cccc(-c4cccc5sc(-c6ccccc6)nc45)c3)n2)cc1 ; c1ccc(-c2cc(-c3ccc4oc5ccccc5c4c3)nc(-c3ccc(-c4cccc(-c5ccc6nc(-c7ccccc7)sc6c5)c4)cc3)n2)cc1]

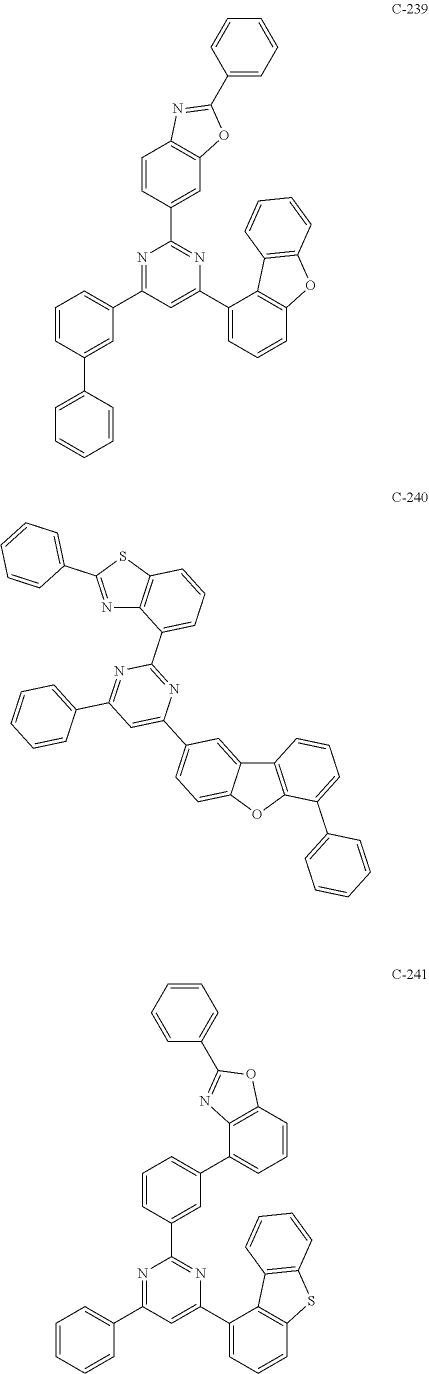 [SMILES: c1ccc(-c2cccc(-c3cc(-c4cccc5oc6ccccc6c45)nc(-c4ccc5nc(-c6ccccc6)oc5c4)n3)c2)cc1 ; c1ccc(-c2cc(-c3ccc4oc5c(-c6ccccc6)cccc5c4c3)nc(-c3cccc4sc(-c5ccccc5)nc34)n2)cc1 ; c1ccc(-c2cc(-c3cccc4sc5ccccc5c34)nc(-c3cccc(-c4cccc5oc(-c6ccccc6)nc45)c3)n2)cc1]

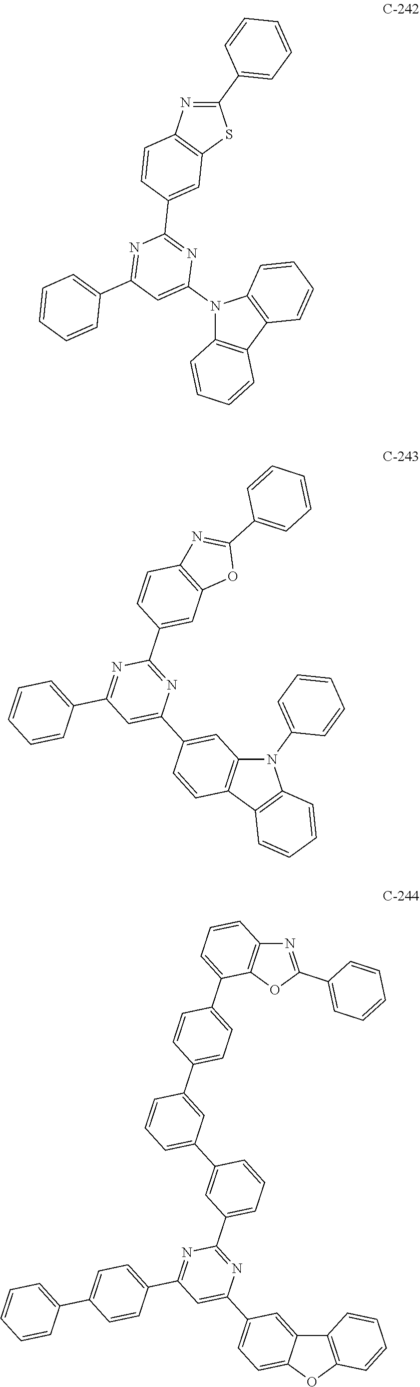 [SMILES: c1ccc(-c2cc(-n3c4ccccc4c4ccccc43)nc(-c3ccc4nc(-c5ccccc5)sc4c3)n2)cc1 ; c1ccc(-c2cc(-c3ccc4c5ccccc5n(-c5ccccc5)c4c3)nc(-c3ccc4nc(-c5ccccc5)oc4c3)n2)cc1 ; c1ccc(-c2ccc(-c3cc(-c4ccc5oc6ccccc6c5c4)nc(-c4cccc(-c5cccc(-c6ccc(-c7cccc8nc(-c9ccccc9)oc78)cc6)c5)c4)n3)cc2)cc1]

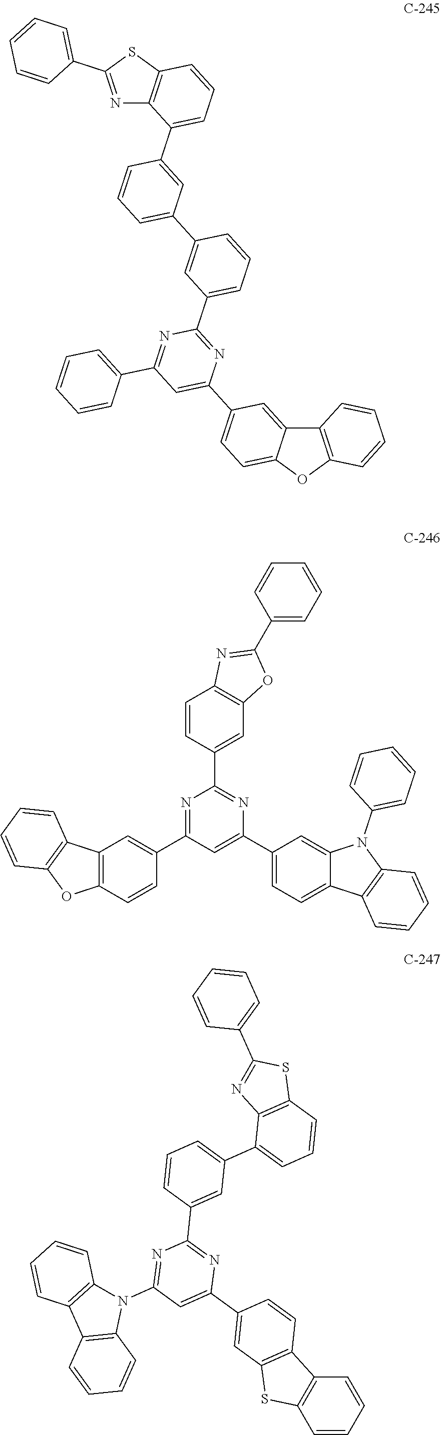 [SMILES: c1ccc(-c2cc(-c3ccc4oc5ccccc5c4c3)nc(-c3cccc(-c4cccc(-c5cccc6sc(-c7ccccc7)nc56)c4)c3)n2)cc1 ; c1ccc(-c2nc3ccc(-c4nc(-c5ccc6oc7ccccc7c6c5)cc(-c5ccc6c7ccccc7n(-c7ccccc7)c6c5)n4)cc3o2)cc1 ; c1ccc(-c2nc3c(-c4cccc(-c5nc(-c6ccc7c(c6)sc6ccccc67)cc(-n6c7ccccc7c7ccccc76)n5)c4)cccc3s2)cc1]

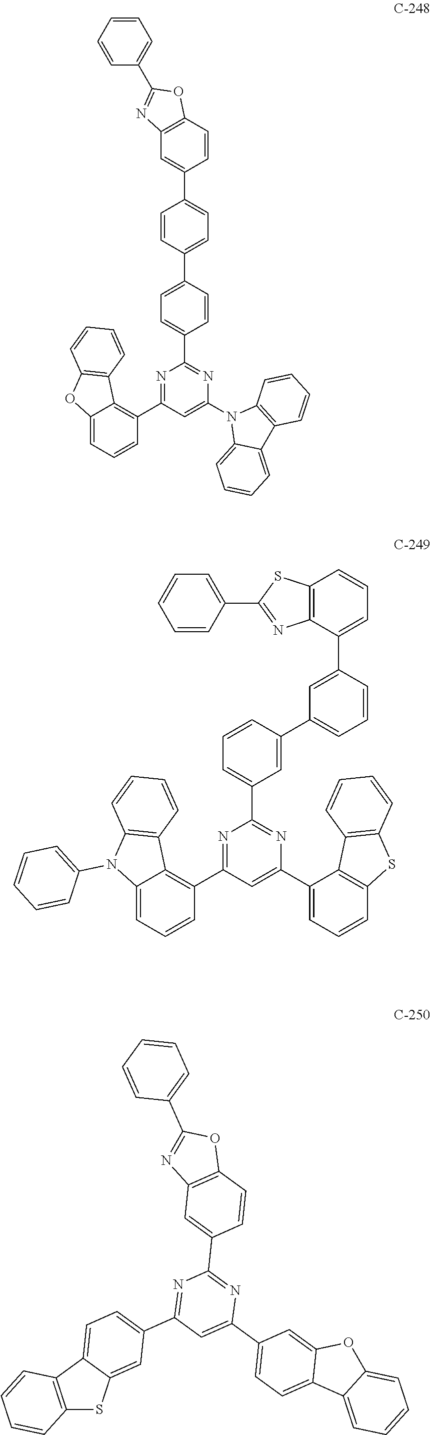 [SMILES: c1ccc(-c2nc3cc(-c4ccc(-c5ccc(-c6nc(-c7cccc8oc9ccccc9c78)cc(-n7c8ccccc8c8ccccc87)n6)cc5)cc4)ccc3o2)cc1 ; c1ccc(-c2nc3c(-c4cccc(-c5cccc(-c6nc(-c7cccc8sc9ccccc9c78)cc(-c7cccc8c7c7ccccc7n8-c7ccccc7)n6)c5)c4)cccc3s2)cc1 ; c1ccc(-c2nc3cc(-c4nc(-c5ccc6c(c5)oc5ccccc56)cc(-c5ccc6c(c5)sc5ccccc56)n4)ccc3o2)cc1]

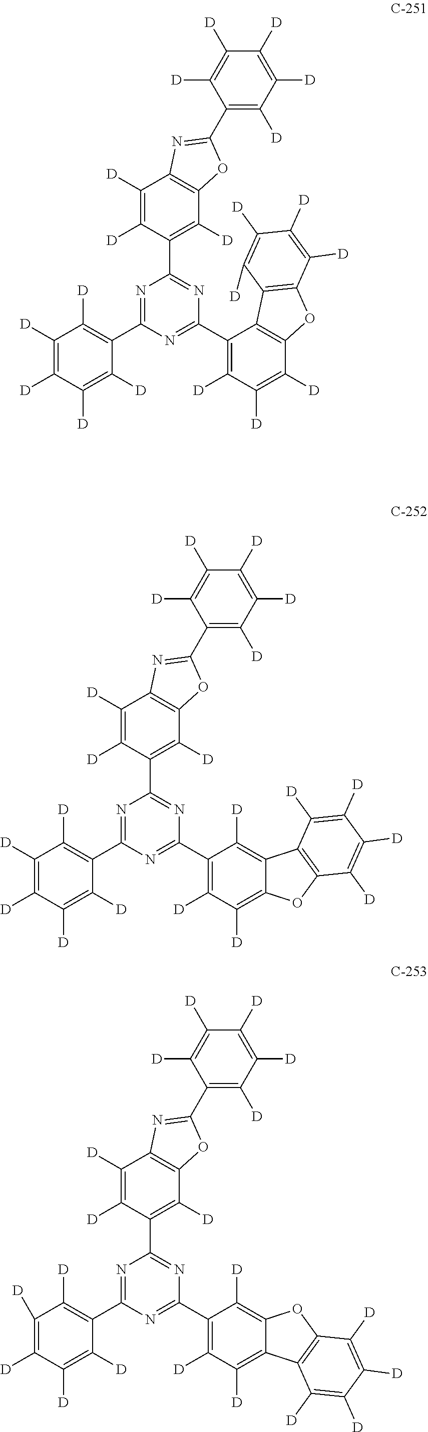

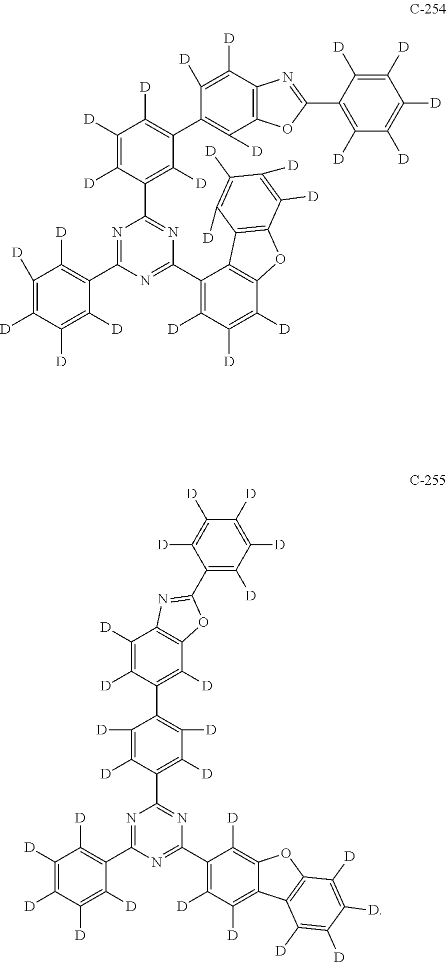

13. An organic electroluminescent device comprising the plurality of host materials according to claim 1.

14. An organic electroluminescent device comprising the organic electroluminescent compound according to claim 11.
